(19) United States
(12) Patent Application Publication
(10) Pub. No.: US 2009/0105292 A9
(43) Pub. Date: Apr. 23, 2009

(54) Glucocorticoid Receptor Modulator Compounds and Methods

(75) Inventors: Lin Zhi, San Diego, CA (US); Robert J. Ardecky, Encinitas, CA (US); Dean Phillips, San Marcos, CA (US); John S. Tyhonas, Chula Vista, CA (US); Donald Karanewsky, Escondido, CA (US); Robert Higuchi, Solana Beach, CA (US); A.R. Hudson, Escondido, CA (US); S.L. Roach, San Diego, CA (US); A.C. Vassar, San Diego, CA (US); Yongkai Li, San Diego, CA (US); Mark E. Adams, San Diego, CA (US); L.J. Valdez, San Diego, CA (US); C. Cuervo, San Diego, CA (US)

Correspondence Address: Bell, Boyd & Lloyd LLP, 3580 Carmel Mountain Road, Suite 200, San Diego, CA, 92130, US

(73) Assignee: LIGAND PHARMACEUTICALS INCORPORATED, SAN DIEGO CALIFORNIA, CA (US)

(21) Appl. No.: 10/589,920

(22) PCT Filed: Feb. 24, 2005
(86) PCT No.: PCT/US05/06627

§ 371 Date: Apr. 20, 2007

Related U.S. Application Data
(60) Provisional application No. 60/548,154, filed on Feb. 25, 2004.

(51) Int. Cl. A61K 31/4741; C07D 491/02

(52) U.S. Cl. 514/285; 546/62

(57) ABSTRACT

Disclosed herein are compounds of Formula I:
and pharmaceutically acceptable salts, esters, amides, and prodrugs thereof. Certain of such compounds are selective glucocorticoid receptor modulators and/or selective glucocorticoid binding agents. Also disclosed are methods of making and using such compounds, including, but not limited to, using such compounds for treating various conditions.

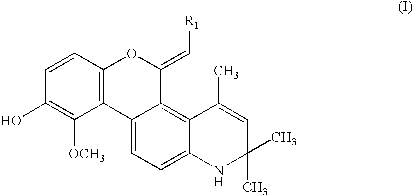

### RELATED APPLICATIONS

Priority is claimed herein under 35 U.S.C. §119(e) to U.S. provisional patent application Ser. No. 60/548,154, filed Feb. 25, 2004, entitled “GLUCOCORTICOID RECEPTOR MODULATOR COMPOUNDS AND METHODS.” The disclosure of the above-referenced provisional application is incorporated herein by reference in its entirety.

### FIELD

Provided herein are compounds that bind to glucocorticoid receptor and/or modulate the activity of glucocorticoid receptor. Also provided are compositions containing such compounds and methods for making and using such compounds and compositions.

### BACKGROUND

Certain intracellular receptors (IRs) have been shown to regulate transcription of certain genes. See e.g., R. M. Evans, Science, 240, 889 (1988). Certain of such IRs are steroid receptors, such as glucocorticoid receptor, androgen receptors, estrogen receptors, mineralocorticoid receptors, and progesterone receptors. Gene regulation by such receptors typically involves binding of an IR by a ligand.

In certain instances, a ligand binds to an IR, forming a receptor/ligand complex. Such a receptor/ligand complex can then translocate to the nucleus of a cell, where it can bind to the DNA of one or more gene regulatory regions. Once bound to the DNA of a particular gene regulatory region, a receptor/ligand complex can modulate the production of the protein encoded by that particular gene. In certain instances, a glucocorticoid receptor/ligand complex regulates expression of certain proteins. In certain instances, a glucocorticoid receptor/ligand complex can interact directly with the DNA of a particular gene regulatory region. In certain instances, a glucocorticoid receptor/ligand complex can interact with other transcription factors, such as activator protein-1 (AP-1) or nuclear factor κB (NFκB). In certain instances, such interactions result in modulation of transcriptional activation.

### SUMMARY

Compounds for use in compositions and methods for modulating the activity of glucocorticoid receptor are provided. The compounds provided herein are substituted quinolines. In one embodiment, the compounds provided herein are agonists of glucocorticoid receptor. In another embodiment, the compounds provided herein are antagonists of glucocorticoid receptor.

In one embodiment, the compounds for use in the compositions and methods provided herein have formula I:

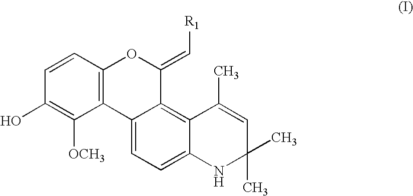

or a pharmaceutically acceptable derivative thereof, wherein R₁ is selected from Formula II, III, and IV:

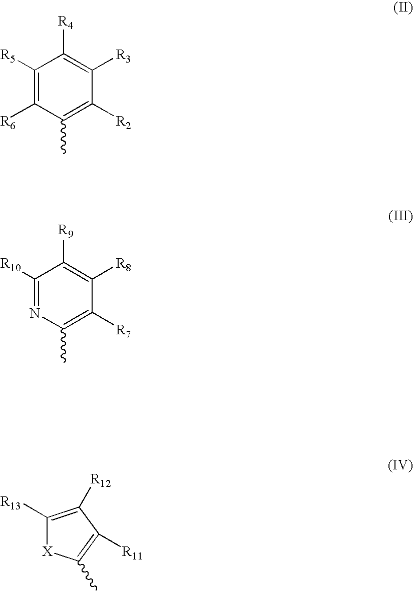

wherein:

R₂ is selected from hydrogen, F, Cl, Br, CN, an optionally substituted alkyl, an optionally substituted alkenyl, an optionally substituted alkynyl, an optionally substituted haloalkyl, an optionally substituted heteroalkyl, —CONR₁₄R₁₅, —OR₁₆, —COR₁₆, —SR₁₆, —SO₂NR₁₄R₁₅, an optionally substituted aryl, an optionally substituted heteroaryl, an optionally substituted heterocyclyl and an optionally substituted cycloalkyl;

R₃ is selected from hydrogen, F, Cl, Br, CN, an optionally substituted alkyl, an optionally substituted alkenyl, an optionally substituted alkynyl, an optionally substituted haloalkyl, an optionally substituted heteroalkyl, —OR₁₆, —SR₁₆, an optionally substituted aryl, an optionally substituted heteroaryl, an optionally substituted heterocyclyl and an optionally substituted cycloalkyl;

R₄ is selected from hydrogen, F, Cl, Br, CN, —OR₁₆, —SR₁₆, an optionally substituted alkyl, an optionally substituted alkenyl, an optionally substituted alkynyl, an optionally substituted haloalkyl, and an optionally substituted heteroalkyl, an optionally substituted aryl, an optionally substituted heteroaryl, an optionally substituted heterocyclyl and an optionally substituted cycloalkyl; or

R₂ and R₃ together form an optionally substituted 5-6 member ring and R₄ is selected from an optionally substituted alkyl, an optionally substituted alkenyl, an optionally substituted alkynyl, an optionally substituted haloalkyl, an optionally substituted heteroalkyl, an optionally substituted aryl, an optionally substituted heteroaryl, an optionally substituted heterocyclyl and an optionally substituted cycloalkyl; or

R₃ and R₄ together form an optionally substituted 4-6 member ring and R₂ is selected from hydrogen, F, Cl, Br, CN, an optionally substituted alkyl, an optionally substituted alkenyl, an optionally substituted alkynyl, an optionally substituted haloalkyl, an optionally substituted heteroalkyl, —CONR₁₄R₁₅, —OR₁₆, —SR₁₆, —SO₂NR₁₄R₁₅, an optionally substituted aryl, an optionally substituted heteroaryl, an optionally substituted heterocyclyl and an optionally substituted cycloalkyl;

R₅ is selected from hydrogen, F, Cl, Br, optionally substituted alkyl, an optionally substituted alkenyl, an optionally substituted alkynyl, —SR₁₆ and —OR₁₆;

R₆ is selected from hydrogen, F, Cl, Br, an optionally substituted alkyl, an optionally substituted alkenyl, an optionally substituted alkynyl;

R₇ is selected from hydrogen, F, Cl, Br, CN, an optionally substituted alkyl, an optionally substituted alkenyl, an optionally substituted alkynyl, an optionally substituted haloalkyl, an optionally substituted heteroalkyl, —CONR₁₄R₁ₛ, —SO₂NR₁₄R₁₅, an optionally substituted aryl, an optionally substituted heteroaryl, an optionally substituted heterocyclyl and an optionally substituted cycloalkyl;

R₈ is selected from hydrogen, F, Cl, Br, CN, an optionally substituted alkyl, an optionally substituted alkenyl, an optionally substituted alkynyl, an optionally substituted haloalkyl, an optionally substituted heteroalkyl, —OR₁₆, —SR₁₆, an optionally substituted aryl, an optionally substituted heteroaryl, an optionally substituted heterocyclyl and an optionally substituted cycloalkyl;

R₉ is selected from hydrogen, F, Cl, Br, CN, an optionally substituted alkyl, an optionally substituted alkenyl, an optionally substituted alkynyl, an optionally substituted haloalkyl, and an optionally substituted heteroalkyl, or

R₇ and R₈ together form an optionally substituted 5-6 member ring and R₉ is selected from hydrogen, F, Cl, Br, CN, an optionally substituted alkyl, an optionally substituted alkenyl, an optionally substituted alkynyl, an optionally substituted haloalkyl, an optionally substituted heteroalkyl, or

R₈ and R₉ together form an optionally substituted 4-6 member ring and R₇ is selected from hydrogen, F, Cl, Br, CN, an optionally substituted alkyl, an optionally substituted alkenyl, an optionally substituted alkynyl, an optionally substituted haloalkyl, an optionally substituted heteroalkyl, —CONR₁₄R₁₅, and an optionally substituted aryl;

R₁₀ is selected from hydrogen, F, Cl, Br, an optionally substituted alkyl, an optionally substituted alkenyl, an optionally substituted alkynyl; and

R₁₁ is selected from hydrogen, F, Cl, Br, CN, an optionally substituted alkyl, an optionally substituted alkenyl, an optionally substituted alkynyl, an optionally substituted haloalkyl, an optionally substituted heteroalkyl, hydroxyliminoalkyl, alkoxyiminoalkyl, aryloxyiminoalkyl, —CONR₁₄R₁₅, SO₂NR₁₄R₁₅, OR₁₆, —SR₁₆, —COR₁₆, an optionally substituted aryl, an optionally substituted heteroaryl, an optionally substituted heterocyclyl and an optionally substituted cycloalkyl;

R₁₂ is selected from hydrogen, F, Cl, Br, CN, an optionally substituted alkyl, an optionally substituted alkenyl, an optionally substituted alkynyl, an optionally substituted haloalkyl, an optionally substituted heteroalkyl, —OR₁₆, —SR₁₆, an optionally substituted aryl, an optionally substituted heteroaryl, an optionally substituted heterocyclyl and an optionally substituted cycloalkyl;

R₁₃ is selected from hydrogen, F, Cl, Br, CN, CONR₁₄R₁₅, an optionally substituted alkyl, an optionally substituted alkenyl, an optionally substituted alkynyl, an optionally substituted haloalkyl, and an optionally substituted heteroalkyl, or

R₁₁ and R₁₂ together form an optionally substituted 5-6 member ring and R₁₃ is selected from hydrogen, F, Cl, Br, CN, CONR₁₄R₁₅, an optionally substituted alkyl, an optionally substituted alkenyl, an optionally substituted alkynyl, an optionally substituted haloalkyl, and an optionally substituted heteroalkyl, or

R₁₂ and R₁₃ together form an optionally substituted 4-6 member ring and R₁₁ is selected from hydrogen, F, Cl, Br, CN, an optionally substituted alkyl, an optionally substituted alkenyl, an optionally substituted alkynyl, an optionally substituted haloalkyl, an optionally substituted heteroalkyl, —CONR₁₄R₁₅, an optionally substituted aryl, an optionally substituted heteroaryl, an optionally substituted heterocyclyl and an optionally substituted cycloalkyl;

R₁₄ and R₁₅ are each independently selected from hydrogen, an optionally substituted alkyl, an optionally substituted alkenyl, an optionally substituted alkynyl, an optionally substituted haloalkyl, an optionally substituted aryl, an optionally substituted heteroaryl, an optionally substituted heterocyclyl, an optionally substituted cycloalkyl and an optionally substituted heteroalkyl, or

R₁₄ and R₁₅ together form an optionally substituted 4-7 member ring;

R₁₆ is selected from hydrogen, an optionally substituted alkyl, an optionally substituted alkenyl, an optionally substituted alkynyl, an optionally substituted haloalkyl, an optionally substituted heteroalkyl, an optionally substituted aryl, an optionally substituted heteroaryl, an optionally substituted heterocyclyl and an optionally substituted cycloalkyl;

X is selected from O, S, and NR₁₇; and

R₁₇ is selected from hydrogen and an optionally substituted alkyl, an optionally substituted alkenyl and an optionally substituted alkynyl;

wherein the substituents on the alkyl, alkenyl, alkynyl, aryl, heteroaryl, heterocyclyl and cycloalkyl groups, when present are selected from one or more, in certain embodiments, 1 to 4, in other embodiments, 1, 2 or 3 substituents, each independently selected from Q¹, wherein Q¹ is halo, pseudohalo, hydroxy, oxo, thia, nitrile, nitro, formyl, mercapto, hydroxycarbonyl, hydroxycarbonylalkyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, imino, hydroxyimino, alkoxyimino, aryloxyimino, aralkoxyimino, alkylazo, arylazo, aralkylazo, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R⁵¹R⁵²R⁵³, P(R⁵⁰)₂, P(═O)(R⁵⁰)₂, OP(═O)(R⁵⁰)2, —NR⁶⁰C(═O)R⁶³, dialkylphosphonyl, alkylarylphosphonyl, diarylphosphonyl, hydroxyphosphonyl, alkylthio, arylthio, perfluoroalkylthio, hydroxylcarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q¹ groups, which substitute atoms in a 1,2 or 1,3 arrangement, together form alkylenedioxy (i.e., —O—(CH₂)_{y}—O—), thioalkylenoxy (i.e., —S—(CH₂)_{y}—O—)or alkylenedithioxy (i.e., —S—(CH₂)_{y}—S—) where y is 1 or 2; or two Q¹ groups, which substitute the same atom, together form alkylene; and

each Q¹ is independently unsubstituted or substituted with one or more substituents, in one embodiment one, two or three substituents, each independently selected from Q²;

each Q² is independently halo, pseudohalo, hydroxy, oxo, thia, nitrile, nitro, formyl, mercapto, hydroxycarbonyl, hydroxycarbonylalkyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R⁵¹R⁵²R⁵³, P(R⁵⁰)₂, P(═O)(R⁵⁰)₂, OP(═O)(R⁵⁰)2, —NR⁶⁰C(═O)R⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q² groups, which substitute atoms in a 1,2 or 1,3 arrangement, together form alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q² groups, which substitute the same atom, together form alkylene;

each Q² is independently unsubstituted or substituted with one or more, in one embodiment one, two or three substituents each independently selected from alkyl, halo and pseudohalo;

R⁵⁰ is hydroxy, alkoxy, aralkoxy, alkyl, heteroaryl, heterocyclyl, aryl or —NR⁷⁰R⁷¹, where R⁷⁰ and R⁷¹ are each independently hydrogen, alkyl, aralkyl, aryl, heteroaryl, heteroaralkyl or heterocyclyl, or R⁷⁰ and R⁷¹ together form alkylene, azaalkylene, oxaalkylene or thiaalkylene;

R⁵¹, R⁵² and R¹³ are each independently hydrogen, alkyl, aryl, aralkyl, heteroaryl, heteroaralkyl, heterocyclyl or heterocyclylalkyl;

R⁶⁰ is hydrogen, alkyl, aryl, aralkyl, heteroaryl, heteroaralkyl, heterocyclyl or heterocyclylalkyl; and

R⁶³ is alkoxy, aralkoxy, alkyl, heteroaryl, heterocyclyl, aryl or —NR⁷⁰R⁷¹; and

wherein

at least one position selected from R₂, R₃, R₄, R₅, and R₆ is not hydrogen;

at least one position selected from R₇, R₈, R₉, and R₁₀ is not hydrogen;

if R₄ is F, then at least one position selected from R₂, R₃, R₅ and R₆ is not hydrogen;

if R₃ is F, then at least one position selected from R₂, R₄, R₅, and R₆ is not hydrogen; and

if any two positions selected from R₂, R₃, R₄, R₅, and R₆ are both F, then at least one of the other three positions selected from R₂, R₃, R⁴, R₅, and R₆ is not hydrogen.

Also of interest are any pharmaceutically-acceptable derivatives, including salts, esters, enol ethers, enol esters, solvates, hydrates and prodrugs of the compounds described herein. Pharmaceutically-acceptable salts, include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc, aluminum, and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates.

Pharmaceutical compositions formulated for administration by an appropriate route and means containing effective concentrations of one or more of the compounds provided herein, or pharmaceutically acceptable derivatives thereof, that deliver amounts effective for the treatment, prevention, or amelioration of one or more symptoms of diseases or disorders that are modulated or otherwise affected by glucocorticoid receptor activity, or in which glucocorticoid receptor activity is implicated, are also provided. The effective amounts and concentrations are effective for ameliorating any of the symptoms of any of the diseases or disorders.

Methods for treatment, prevention, or amelioration of one or more symptoms of diseases or disorders mediated by or in which glucocorticoid receptor activity is implicated, are provided.

Methods of modulating the activity of glucocorticoid receptor using the compounds and compositions provided herein are also provided. The compounds and compositions provided herein are active in assays that measure the activity of glucocorticoid receptor including the assays provided herein. These methods include inhibiting and up-regulating the activity of glucocorticoid receptor. Certain of such methods are effected by contacting a glucocorticoid receptor with one or more compounds provided herein.

Provided herein are methods for identifying a compound that is capable of modulating activity of a glucocorticoid receptor. The methods are effected by: a) contacting a cell expressing the glucocorticoid receptor with a compound provided herein; and b) monitoring an effect of the compound upon the cell. In certain of such embodiments, the compound is derived from a quinoline. In certain embodiments, the compound is a 6-arylquinoline.

In certain embodiments, provided herein are methods for treating a subject evidencing a glucocorticoid receptor mediated disease or disorder, or a disease or disorders in which the activity of a glucocorticoid receptor is implicated by administering to the subject a compound provided herein.

In practicing the methods, effective amounts of the compounds or compositions containing therapeutically effective concentrations of the compounds, which are formulated for systemic delivery, including parenteral, oral, or intravenous delivery, or for local or topical application, for the treatment of glucocorticoid receptor mediated diseases or disorders, or diseases or disorders in which the activity of a glucocorticoid receptor is implicated, including, but not limited to, inflammatory diseases, autoimmune diseases, hyperproliferative diseases, and other such disease. Exemplary of these diseases are inflammatory diseases, such as rheumatoid arthritis, asthma (acute and/or chronic), lupus, osteoarthritis, rhinosinusitis, inflammatory bowel disease, polyarteritis nodosa, Wegener's granulomatosis, giant cell arteritis, allergic rhinitis, urticaria, hereditary angioedema, chronic obstructive pulmonary disease, tendonitis, bursitis, autoimmune chronic active hepatitis, cirrhosis, transplant rejection, psoriasis, dermatitus, autoimmune disorders, malignancies (e.g., leukemia, myelomas, lymphomas), acute adrenal insufficiency, congenital adrenal hyperplasia, rheumatic fever, granulomatous disease, immune proliferation/apotosis, hypothalamic-pituitary-adrenal (HPA) axis suppression and regulation, hypercortisolemia, modulation of the Th1/Th2 cytokine balance, chronic kidney disease, stroke and spinal cord injury, hypercalcemia, hyperglycemia, cerebral edema, thrombocytopenia, Little's syndrome, Addison's disease, cystic fibrosis, myasthenia gravis, autoimmune hemolytic anemia, uveitis, pemphigus vulgaris, multiple sclerosis, nasal polyps, sepsis, infections (e.g., bacterial, viral, rickettsial, parasitic), type II diabetes, obesity, metabolic syndrome, depression, schizophrenia, mood disorders, Cushing's syndrome, anxiety, sleep disorders, memory and learning enhancement, or glucocorticoid-induced glaucoma, are administered to an individual exhibiting the symptoms of these diseases or disorders. The amounts are effective to ameliorate or eliminate one or more symptoms of the diseases or disorders.

Also provided are pharmaceutical compositions containing: i) a physiologically acceptable carrier, diluent, or excipient, or a combination thereof; and ii) one or more compounds provided herein. The compositions can be formulated for single dosage administration or for multiple dosages.

Articles of manufacture containing packaging material, within the packaging material a compound or composition, or pharmaceutically acceptable derivative thereof, which is effective for modulating the activity of glucocorticoid receptor, or for treatment, prevention or amelioration of one or more symptoms of glucocorticoid receptor mediated diseases or disorders, or diseases or disorders in which glucocorticoid receptor activity is implicated, and a label that indicates that the compound or composition, or pharmaceutically acceptable derivative thereof, is used for modulating the activity of glucocorticoid receptor, or for treatment, prevention or amelioration of one or more symptoms of glucocorticoid receptor mediated diseases or disorders, or diseases or disorders in which glucocorticoid receptor activity is implicated, are provided.

### DETAILED DESCRIPTION OF EMBODIMENTS

A. Definitions

Unless defined otherwise, all technical and scientific terms used herein have the same meaning as is commonly understood by one of skill in the art to which the claimed subject matter belongs. All patents, patent applications, published applications and publications, Genbank sequences, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information. It is to be understood that both the foregoing general description and the following detailed description are exemplary and explanatory only and are not restrictive of the products, methods and other subject matter provided herein. In this application, the use of the singular includes the plural unless specifically stated otherwise. In this application, the use of “or” means “and/or” unless stated otherwise. Furthermore, use of the term “including” as well as other forms, such as “includes,” and “included,” is not limiting.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

Unless specific definitions are provided, the nomenclatures utilized in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits according to manufacturer's specifications or as commonly accomplished in the art or as described herein. The foregoing techniques and procedures can be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), which is incorporated herein by reference for any purpose.

As used herein, the following terms are defined with the following meanings, unless explicitly stated otherwise.

As used herein, the term “selective binding compound” refers to a compound that selectively binds to any portion of one or more target receptors.

As used herein, the term “selective glucocorticoid receptor binding compound” refers to a compound that selectively binds to any portion of a glucocorticoid receptor.

As used herein, the term “selectively binds” refers to the ability of a selective binding compound to bind to a target receptor with greater affinity than it binds to a non-target receptor. In certain embodiments, specific binding refers to binding to a target with an affinity that is at least 10, 50, 100, 250, 500, 1000 or more times greater than the affinity for a non-target.

As used herein, the term “target receptor” refers to a molecule or a portion of a receptor capable of being bound by a selective binding compound. In certain embodiments, a target receptor is a glucocorticoid receptor.

As used herein, the terms “treating” or “treatment” encompass either or both responsive and prophylaxis measures, e.g., designed to inhibit or delay the onset of the disease or disorder, achieve a full or partial reduction of the symptoms or disease state, and/or to alleviate, ameliorate, lessen, or cure the disease or disorder and/or its symptoms. Treatment also encompasses any pharmaceutical use of the compositions herein, such as use for treating a gluococorticoid mediated diseases or disorders.

As used herein, amelioration of the symptoms of a particular disorder by administration of a particular compound or pharmaceutical composition refers to any lessening, whether permanent or temporary, lasting or transient that can be attributed to or associated with administration of the composition.

As used herein, the term “modulator” refers to a compound that alters an activity of a molecule. For example, a modulator can cause an increase or decrease in the magnitude of a certain activity of a molecule compared to the magnitude of the activity in the absence of the modulator. In certain embodiments, a modulator is an inhibitor, which decreases the magnitude of one or more activities of a molecule. In certain embodiments, an inhibitor completely prevents one or more activities of a molecule. In certain embodiments, a modulator is an activator, which increases the magnitude of at least one activity of a molecule. In certain embodiments the presence of a modulator results in an activity that does not occur in the absence of the modulator.

As used herein, the term “selective modulator” refers to a compound that selectively modulates a target activity.

As used herein, the term “selective glucocorticoid receptor modulator” refers to a compound that selectively modulates at least one activity associated with a glucocorticoid receptor.

As used herein, the term “selectively modulates” refers to the ability of a selective modulator to modulate a target activity to a greater extent than it modulates a non-target activity. In certain embodiments the target activity is selectively modulated by, for example about 2 fold up to more than about 500 folds, in some embodiments, about 2, 5, 10, 50, 100, 150, 200, 250, 300, 350, 400, 450 or more than 500 folds.

As used herein, the term “target activity” refers to a biological activity capable of being modulated by a selective modulator. Certain exemplary target activities include, but are not limited to, binding affinity, signal transduction, enzymatic activity, tumor growth, and inflammation or inflammation-related processes.

As used herein, the term “receptor mediated activity” refers any biological activity that results, either directly or indirectly, from binding of a ligand to a receptor.

As used herein, the term “agonist” refers to a compound, the presence of which results in a biological activity of a receptor that is the same as the biological activity resulting from the presence of a naturally occurring ligand for the receptor.

As used herein, the term “partial agonist” refers to a compound the presence of which results in a biological activity of a receptor that is of the same type as that resulting from the presence of a naturally occurring ligand for the receptor, but of a lower magnitude.

As used herein, the term “antagonist” refers to a compound, the presence of which results in a decrease in the magnitude of a biological activity of a receptor. In certain embodiments, the presence of an antagonist results in complete inhibition of a biological activity of a receptor.

As used herein, the IC₅₀ refers to an amount, concentration or dosage of a particular test compound that achieves a 50% inhibition of a maximal response, such as modulation of glucocorticoid activity, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

As used herein, C₁-Cₓ includes C₁-C₂, C₁-C₃ . . . C₁-Cₓ.

As used herein, the term “alkyl” refers to an aliphatic hydrocarbon group. An alkyl group can be a “saturated alkyl,” which means that it does not contain any alkene or alkyne groups. An alkyl group can be an “unsaturated alkyl,” which means that it contains at least one alkene or alkyne group. An alkyl, whether saturated or unsaturated, can be branched, straight chain, or cyclic.

In certain embodiments, an alkyl contains 1 to 20 carbon atoms (whenever it appears herein, a numerical range such as “1 to 20” refers to each integer in the given range; e.g., “1 to 20 carbon atoms” means that an alkyl group can contain only 1 carbon atom, 2 carbon atoms, 3 carbon atoms, etc., up to and including 20 carbon atoms, although the term “alkyl” also includes instances where no numerical range of carbon atoms is designated).

As used herein, the term “lower alkyl” refers to an alkyl containing 1 to 5 carbon atoms. The term “medium alkyl” refers to an alkyl containing 5 to 10 carbon atoms. An alkyl can be designated as “C₁-C₄ alkyl” or similar designations. By way of example only, “C₁-C₄ alkyl” indicates an alkyl having one, two, three, or four carbon atoms, i.e., the alkyl is selected from among methyl, ethyl, propyl, iso-propyl, n-butyl, iso-butyl, sec-butyl, and t-butyl. Thus C₁-C₄ includes C₁-C₂ and C₁-C₃ alkyl. Alkyls can be substituted or unsubstituted. Alkyls include, but are not limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like, each of which can be optionally substituted.

As used herein, the term “alkenyl” refers to an alkyl group containing at least one carbon-carbon double bond.

As used herein, the term “alkynyl” refers to an alkyl group containing at least one carbon-carbon triple bond.

As used herein, the term “haloalkyl” refers to an alkyl in which at least one hydrogen atom is replaced with a halogen atom. In certain of the embodiments in which two or more hydrogen atom are replaced with halogen atoms, the halogen atoms are all the same as one another. In certain of such embodiments, the halogen atoms are not all the same as one another.

As used herein, the term “heteroalkyl” refers to a group containing an alkyl and one or more heteroatoms. Certain heteroalkyls are acylalkyls, in which the one or more heteroatoms are within an alkyl chain. Examples of heteroalkyls include, but are not limited to, CH₃C(═O)CH₂—, CH₃C(═O)CH₂CH₂—, CH₃CH₂C(═O)CH₂CH₂—, CH₃C(═O)CH₂CH₂CH₂—, CH₃OCH₂CH₂—, CH₃NHCH₂—, and the like.

As used herein, the term “heterohaloalkyl” refers to a heteroalkyl in which at least one hydrogen atom is replaced with a halogen atom.

As used herein, the term “carbocycle” refers to a group containing a covalently closed ring, wherein each of the atoms forming the ring is a carbon atom. Carbocylic rings can be formed by three, four, five, six, seven, eight, nine, or more than nine carbon atoms. Carbocycles can be optionally substituted.

As used herein, the term “heterocycle” refers to a group containing a covalently closed ring wherein at least one atom forming the ring is a heteroatom. Heterocyclic rings can be formed by three, four, five, six, seven, eight, nine, or more than nine atoms. Heterocycles can be optionally substituted. Binding to a heterocycle can be at a heteroatom or via a carbon atom. For example, binding for benzo-fused derivatives, can be via a carbon of the benzenoid ring.

As used herein, the term “heteroatom” refers to an atom other than carbon or hydrogen. Heteroatoms are typically independently selected from oxygen, sulfur, nitrogen, and phosphorus, but are not limited to those atoms. In embodiments in which two or more heteroatoms are present, the two or more heteroatoms can all be the same as one another, or some or all of the two or more heteroatoms can each be different from the others.

As used herein, the term “aromatic” refers to a group containing a covalently closed ring having a delocalized π-electron system. Aromatic rings can be formed by three, four, five, six, seven, eight, nine, or more than nine atoms. Aromatics can be optionally substituted. Examples of aromatic groups include, but are not limited to phenyl, naphthalenyl, phenanthrenyl, anthracenyl, tetralinyl, fluorenyl, indenyl, and indanyl. The term aromatic includes, for example, benzenoid groups, connected via one of the ring-forming carbon atoms, and optionally carrying one or more substituents selected from an aryl, a heteroaryl, a cycloalkyl, a non-aromatic heterocycle, a halo, a hydroxy, an amino, a cyano, a nitro, an alkylamido, an acyl, a C₁₋₆ alkoxy, a C₁₋₆ alkyl, a hydroxyC₁₋₆alkyl, a aminoC₁₋₆ alkyl, a C₁₋₆alkylamino, an alkylsulfenyl, an alkylsulfinyl, an alkylsulfonyl, an sulfamoyl, or a trifluoromethyl. In certain embodiments, an aromatic group is substituted at one or more of the para, meta, and/or ortho positions. Examples of aromatic groups containing substitutions include, but are not limited to, phenyl, 3-halophenyl, 4-halophenyl, 3-hydroxyphenyl, 4-hydroxyphenyl, 3-aminophenyl, 4-aminophenyl, 3-methylphenyl, 4-methylphenyl, 3-methoxyphenyl, 4-methoxyphenyl, 4-trifluoromethoxyphenyl, 3-cyanophenyl, 4-cyanophenyl, dimethylphenyl, naphthyl, hydroxynaphthyl, hydroxymethylphenyl, (trifluoromethyl)phenyl, alkoxyphenyl, 4-morpholin-4-ylphenyl, 4-pyrrolidin-1-ylphenyl, 4-pyrazolylphenyl, 4-triazolylphenyl, and 4-(2-oxopyrrolidin-1-yl)phenyl.

As used herein, the term “aryl” refers to an aromatic group wherein each of the atoms forming the ring is a carbon atom. Aryl rings can be formed by three, four, five, six, seven, eight, nine, or more than nine carbon atoms. Aryl groups can be optionally substituted.

As used herein, the term “heteroaryl” refers to an aromatic group in which at least one atom forming the aromatic ring is a heteroatom. Heteroaryl rings can be formed by three, four, five, six, seven, eight, nine and more than nine atoms. Heteroaryl groups can be optionally substituted. Examples of heteroaryl groups include, but are not limited to, aromatic C₃₋₈ heterocyclic groups containing one oxygen or sulfur atom or up to four nitrogen atoms, or a combination of one oxygen or sulfur atom and up to two nitrogen atoms, and their substituted as well as benzo- and pyrido-fused derivatives, for example, connected via one of the ring-forming carbon atoms. In certain embodiments, heteroaryl groups are optionally substituted with one or more substituents, independently selected from halo, hydroxy, amino, cyano, nitro, alkylamido, acyl, C₁₋₆-alkoxy, C₁₋₆-alkyl, hydroxy-C₁₋₆-alkyl, aminoC₁₋₆-alkyl, C₁₋₆-alkylamino, alkylsulfenyl, alkylsulfinyl, alkylsulfonyl, sulfamoyl, or trifluoromethyl. As in all examples herein C₁-Cₓ includes C₁-C₂, C₁-C₃ . . . C₁-Cₓ. Examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quinoline, isoquinoline, pyridazine, pyrimidine, purine and pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline, and quinoxaline. In some embodiments, the substituents are halo, hydroxy, cyano, O—C₁₋₆-alkyl, C₁₋₆-alkyl, hydroxy-C₁₋₆-alkyl, and amino-C₁₋₆-alkyl.

As used herein, the term “non-aromatic ring” refers to a group containing a covalently closed ring that does not have a delocalized π-electron system.

As used herein, the term “cycloalkyl” refers to a group containing a non-aromatic ring wherein each of the atoms forming the ring is a carbon atom. Cycloalkyl rings can be formed by three, four, five, six, seven, eight, nine, or more than nine carbon atoms. Cycloalkyls can be optionally substituted. In certain embodiments, a cycloalkyl contains one or more unsaturated bonds. Examples of cycloalkyls include, but are not limited to, cyclopropane, cyclobutane, cyclopentane, cyclopentene, cyclopentadiene, cyclohexane, cyclohexene, 1,3-cyclohexadiene, 1,4-cyclohexadiene, cycloheptane, and cycloheptene.

As used herein, the term “non-aromatic heterocycle” refers to a group containing a non-aromatic ring wherein one or more atoms forming the ring is a heteroatom. Non-aromatic heterocyclic rings can be formed by three, four, five, six, seven, eight, nine, or more than nine atoms. Non-aromatic heterocycles can be optionally substituted. In certain embodiments, non-aromatic heterocycles contain one or more carbonyl or thiocarbonyl groups such as, for example, oxo- and thio-containing groups. Examples of non-aromatic heterocycles include, but are not limited to, lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane.

As used herein, the term “arylalkyl” refers to a group containing an aryl group bound to an alkyl group.

As used herein, the term “carbocycloalkyl” refers to a group containing a carbocyclic cycloalkyl ring. Carbocycloalkyl rings can be formed by three, four, five, six, seven, eight, nine, or more than nine carbon atoms. Carbocycloalkyl groups can be optionally substituted.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g., aryls and heteroaryls), and non-aromatics (e.g., cycloalkyls and non-aromatic heterocycles). Rings can be optionally substituted. Rings can form part of a ring system.

As used herein, the term “ring system” refers to two or more rings, wherein two or more of the rings are fused. The term “fused” refers to structures in which two or more rings share one or more bonds.

As used herein, the substituent “R” appearing by itself and without a number designation refers to a substituent selected from alkyl, cycloalkyl, aryl, heteroaryl (bonded through a ring carbon) and non-aromatic heterocycle (bonded through a ring carbon).

The term “O-carboxy” refers to a group of formula RC(═O)O—.

The term “C-carboxy” refers to a group of formula —C(═O)OR.

The term “acetyl” refers to a group of formula —C(═O)CH₃.

The term “trihalomethanesulfonyl” refers to a group of formula X₃CS(═O)₂— where X is a halogen.

The term “cyano” refers to a group of formula —CN.

The term “isocyanato” refers to a group of formula —NCO.

The term “thiocyanato” refers to a group of formula —CNS.

The term “isothiocyanato” refers to a group of formula —NCS.

The term “sulfinyl” refers to a group of formula —S(═O)—R.

The term “S-sulfonamido” refers to a group of formula —S(═O)₂NR₂.

The term “N-sulfonamido” refers to a group of formula RS(═O)₂NH—.

The term “trihalomethanesulfonamido” refers to a group of formula X₃CS(═O)₂NR—.

The term “O-carbamyl” refers to a group of formula —OC(═O)—NR₂.

The term “N-carbamyl” refers to a group of formula ROC(═O)NH—.

The term “O-thiocarbamyl” refers to a group of formula —OC(═S)—NR₂.

The term “N-thiocarbamyl” refers to a group of formula ROC(═S)NH—.

The term “C-amido” refers to a group of formula —C(═O)—NR₂.

The term “N-amido” refers to a group of formula RC(═O)NH—.

The term “ester” refers to a chemical moiety with formula —(R)ₙ—COOR′, where R and R′ are independently selected from alkyl, cycloalkyl, aryl, heteroaryl (bonded through a ring carbon) and non-aromatic heterocycle (bonded through a ring carbon), where n is 0 or 1.

As used herein, the term “amide” refers to a chemical moiety with the formula —(R)ₙ—C(O)NHR′ or —(R)ₙ—NHC(O)R′, where R and R′ are independently selected from alkyl, cycloalkyl, aryl, heteroaryl (bonded through a ring carbon) and heteroalicyclic (bonded through a ring carbon), where n is 0 or 1. In certain embodiments, an amide can be an amino acid or a peptide.

As used herein, the terms “amine,” “hydroxy,” and “carboxyl” include such groups that have been esterified or amidified. Procedures and specific groups used to achieve esterification and amidification are known to those of skill in the art and can readily be found in reference sources such as Greene and Wuts, Protective Groups in Organic Synthesis, 3^{rd} Ed., John Wiley & Sons, New York, N.Y., 1999, which is incorporated herein in its entirety.

Throughout the specification, groups and substituents thereof can be chosen by one skilled in the field to provide stable moieties and compounds.

Unless otherwise indicated, the term “optionally substituted,” refers to a group in which none, one, or more than one of the hydrogen atoms has been replaced with one or more group(s) individually and independently selected from: cycloalkyl, aryl, heteroaryl, non-aromatic heterocycle, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino, including mono and di substituted amino groups, and the protected derivatives of amino groups. Such protective derivatives (and protecting groups that can form such protective derivatives) are known to those of skill in the art and can be found in references such as Greene and Wuts, above. In embodiments in which two or more hydrogen atoms have been substituted, the substituent groups can together form a ring.

As used herein, the term “carrier” refers to a compound that facilitates the incorporation of another compound into cells or tissues. For example, dimethyl sulfoxide (DMSO) is a commonly used carrier for improving incorporation of certain organic compounds into cells or tissues.

As used herein, the term “pharmaceutical agent” refers to a chemical compound or composition capable of inducing a desired therapeutic effect in a patient. In certain embodiments, a pharmaceutical agent contains an active agent, which is the agent that induces the desired therapeutic effect. In certain embodiments, a pharmaceutical agent is a prodrug. In certain embodiments, a pharmaceutical agent contains inactive ingredients such as carriers, excipients, and the like.

As used herein, the term “therapeutically effective amount” refers to an amount of a pharmaceutical agent sufficient to achieve a desired therapeutic effect.

As used herein, a “prodrug” refers to an pharmaceutical agent that is converted from a less active form into a corresponding more active form in vivo. A prodrug is a compound that, upon in vivo administration, is metabolized by one or more steps or processes or otherwise converted to the biologically, pharmaceutically or therapeutically active form of the compound. To produce a prodrug, the pharmaceutically active compound is modified such that the active compound will be regenerated by metabolic processes. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

As used herein, the term “pharmaceutically acceptable” refers to a formulation of a compound that does not significantly abrogate the biological activity, a pharmacological activity and/or other properties of the compound when the formulated compound is administered to a patient. In certain embodiments, a pharmaceutically acceptable formulation does not cause significant irritation to a patient.

As used herein, pharmaceutically acceptable derivatives of a compound include salts, esters, enol ethers, enol esters, acetals, ketals, orthoesters, hemiacetals, hemiketals, acids, bases, solvates, hydrates or prodrugs thereof. Such derivatives can be readily prepared by those of skill in this art using known methods for such derivatization. The compounds produced can be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, heteroaryl, aralkyl, heteroaralkyl, cycloalkyl ar heterocyclyl. Pharmaceutically acceptable enol esters include, but are not limited to, derivatives of formula C═C(OC(O)R) where R is hydrogen, alkyl, alkenyl, alkynyl, aryl, heteroaryl, aralkyl, heteroaralkyl, cycloalkyl ar heterocyclyl. Pharmaceutically acceptable solvates and hydrates are complexes of a compound with one or more solvent or water molecules, or 1 to about 100, or 1 to about 10, or one to about 2, 3 or 4 solvent or water molecules.

It is to be understood that the compounds provided herein can contain chiral centers. Such chiral centers can be of either the (R) or (S) configuration, or can be a mixture thereof. Thus, the compounds provided herein can be enantiomerically pure, or be stereoisomeric or diastereomeric mixtures.

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Thus, substantially pure object species (e.g., compound) is the predominant species present (i.e., on a molar basis it is more abundant than any other individual species in the composition). In certain embodiments, a substantially purified fraction is a composition wherein the object species contains at least about 50 percent (on a molar basis) of all species present. In certain embodiments, a substantially pure composition will contain more than about 50%, 60%, 70%, 80%, 85%, 90%, 95%, or 99% of all species present in the composition. In certain embodiments, a substantially pure composition will contain more than about 80%, 85%, 90%, 95%, or 99% of all species present in the composition. Methods for purification of the compounds to produce substantially chemically pure compounds are known to those of skill in the art. A substantially chemically pure compound can, however, be a mixture of stereoisomers. In such instances, further purification might increase the specific activity of the compound. The instant disclosure is meant to include all such possible isomers, as well as their racemic and optically pure forms. Optically active (+) and (−), (R)- and (S)-, or (D)- and (L)-isomers can be prepared using chiral synthons or chiral reagents, or resolved using conventional techniques, such as reverse phase HPLC. When the compounds described herein contain olefinic double bonds or other centers of geometric asymmetry, and unless specified otherwise, it is intended that the compounds include both E and Z geometric isomers. Likewise, all tautomeric forms are also intended to be included.

As used herein, the term “co-administer” refers to administering more than one pharmaceutical agent to a patient. In certain embodiments, co-administered pharmaceutical agents are administered together in a single dosage unit. In certain embodiments, co-administered pharmaceutical agents are administered separately. In certain embodiments, co-administered pharmaceutical agents are administered at the same time. In certain embodiments, co-administered pharmaceutical agents are administered at different times.

As used herein “subject” is an animal, typically a mammal, including human.

As used herein, the term “patient” includes human and animal subjects.

As used herein, the term “tissue-selective” refers to the ability of a compound to modulate a biological activity in one tissue to a greater or lesser degree than it modulates a biological activity in another tissue. The biological activities in the different tissues can be the same or they can be different. The biological activities in the different tissues can be mediated by the same type of target receptor. For example, in certain embodiments, a tissue-selective compound can modulate a glucocorticoid receptor mediated biological activity in one tissue and fail to modulate, or modulate to a lesser degree, a glucocorticoid receptor mediated biological activity in another tissue type.

As used herein, the term “monitoring” refers to observing an effect or absence of any effect. In certain embodiments, one monitors cells after contacting those cells with a compound provided herein. Examples of effects that can be monitored include, but are not limited to, changes in cell phenotype, cell proliferation, glucocorticoid receptor activity, or the interaction between a glucocorticoid receptor and a natural binding partner.

As used herein, the term “cell phenotype” refers to physical or biological characteristics. Examples of characteristics that constitute phenotype include, but are not limited to, cell size, cell proliferation, cell differentiation, cell survival, apoptosis (cell death), or the utilization of a metabolic nutrient (e.g., glucose uptake). Certain changes or the absence of changes in cell phenotype are readily monitored using techniques known in the art.

As used herein, the term “cell proliferation” refers to the rate at which cells divide. The number of cells growing in a vessel can be quantified by a person skilled in the art (e.g., by counting cells in a defined area using a light microscope, or by using laboratory apparatus that measure the density of cells in an appropriate medium). One skilled in that art can calculate cell proliferation by determining the number of cells at two or more times.

As used herein, the term “contacting” refers to bringing two or more materials into close enough proximity that they can interact. In certain embodiments, contacting can be accomplished in a vessel such as a test tube, a petri dish, or the like. In certain embodiments, contacting can be performed in the presence of additional materials. In certain embodiments, contacting can be performed in the presence of cells. In certain of such embodiments, one or more of the materials that are being contacted can be inside a cell. Cells can be alive or can be dead. Cells can or can not be intact.

B. Compounds

Certain compounds that bind to glucocorticoid receptor and/or modulate an activity of such receptors play a role in health (e.g., normal growth, development, and/or absence of disease). In certain embodiments, selective glucocorticoid receptor modulators and/or binding compounds are useful for treating any of a variety of diseases or conditions.

Certain compounds have been previously described as receptor modulators. See e.g., U.S. Pat. Nos. 5,693,646; 6,380,207; 6,506,766; 5,688,810; 5,696,133; Zhi, et. al. Bioorganic & Medicinal Chemistry Letters 2000, 10, 415-418; Pooley, et. al., J. Med. Chem. 1998, 41, 3461, the entire disclosures of which are incorporated herein in their entirety.

The compounds provided herein are glucocorticoid receptor modulators. In certain embodiments, the compounds provided are selective glucocorticoid receptor modulators. In certain embodiments, the compounds provided are selective glucocorticoid receptor binding agents. In certain embodiments, selective glucocorticoid modulators are agonists, partial agonists, and/or antagonists for the glucocorticoid receptor.

In certain embodiments, the compounds provided are of Formula I:

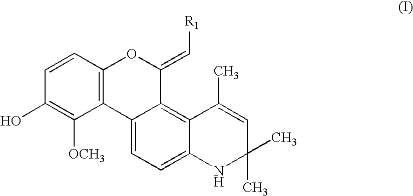

or a pharmaceutically acceptable salt, ester, amide, or prodrug thereof, wherein R₁ is selected from Formula II, III, and IV:

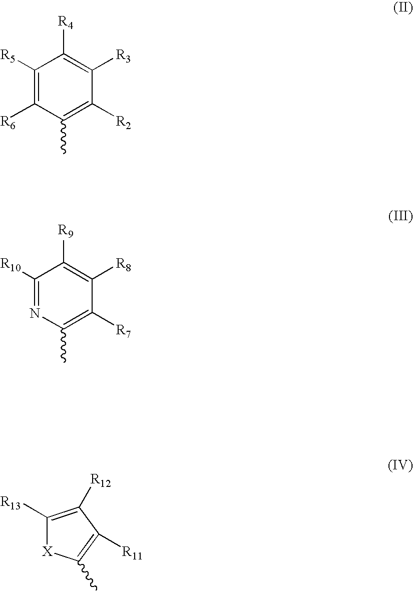

wherein:

R₂ is selected from hydrogen, F, Cl, Br, CN, an optionally substituted C₁-C₄ alkyl, an optionally substituted C₂-C₄ alkenyl, an optionally substituted C₂-C₄ alkynyl, an optionally substituted C₁-C₄ haloalkyl, an optionally substituted C₁-C₄ heteroalkyl, —CONR₁₄R₁₅, —OR₁₆, —COR₁₆, —SR₁₆, —SO₂NR₁₄R₁₅, an optionally substituted aryl, an optionally substituted heteroaryl, an optionally substituted heterocyclyl and an optionally substituted cycloalkyl;

R₃ is selected from hydrogen, F, Cl, Br, CN, an optionally substituted C₁-C₄ alkyl, an optionally substituted C₂-C₄ alkenyl, an optionally substituted C₂-C₄ alkynyl, an optionally substituted C₁-C₄ haloalkyl, an optionally substituted C₁-C₄ heteroalkyl, —OR₁₆, —SR₁₆, an optionally substituted aryl, an optionally substituted heteroaryl, an optionally substituted heterocyclyl and an optionally substituted cycloalkyl;

R₄ is selected from hydrogen, F, Cl, Br, CN, —OR₁₆, an optionally substituted C₁-C₄ alkyl, an optionally substituted C₂-C₄ alkenyl, an optionally substituted C₂-C₄ alkynyl, an optionally substituted C₁-C₄ haloalkyl, and an optionally substituted C₁-C₄ heteroalkyl, an optionally substituted aryl, an optionally substituted heteroaryl, an optionally substituted heterocyclyl and an optionally substituted cycloalkyl; or

R₂ and R₃ together form an optionally substituted 5-6 member ring and R₄ is selected from hydrogen, F, Cl, Br, CN, —OR₁₆, an optionally substituted C₁-C₄ alkyl, an optionally substituted C₂-C₄ alkenyl, an optionally substituted C₂-C₄ alkynyl, an optionally substituted C₁-C₄ haloalkyl, an optionally substituted C₁-C₄ heteroalkyl, an optionally substituted aryl, an optionally substituted heteroaryl, an optionally substituted heterocyclyl and an optionally substituted cycloalkyl; or

R₃ and R₄ together form an optionally substituted 4-6 member ring and R₂ is selected from hydrogen, F, Cl, Br, CN, an optionally substituted C₁-C₄ alkyl, an optionally substituted C₂-C₄ alkenyl, an optionally substituted C₂-C₄ alkynyl, an optionally substituted C₁-C₄ haloalkyl, an optionally substituted C₁-C₄ heteroalkyl, —CONR₁₄R₁₅, —OR₆, —SR₁₆, —SO₂NR₁₄R₁₅, an optionally substituted aryl, an optionally substituted heteroaryl, an optionally substituted heterocyclyl and an optionally substituted cycloalkyl;

R₅ is selected from hydrogen, F, Cl, Br, optionally substituted C₁-C₄ alkyl, an optionally substituted C₂-C₄ alkenyl, an optionally substituted C₂-C₄ alkynyl, and —OR₁₆;

R₆ is selected from hydrogen, F, Cl, Br, an optionally substituted C₁-C₄ alkyl, an optionally substituted C₂-C₄ alkenyl, an optionally substituted C₂-C₄ alkynyl;

R₇ is selected from hydrogen, F, Cl, Br, CN, an optionally substituted C₁-C₄ alkyl, an optionally substituted C₂-C₄ alkenyl, an optionally substituted C₂-C₄ alkynyl, an optionally substituted C₁-C₄ haloalkyl, an optionally substituted C₁-C₄ heteroalkyl, —CONR₁₄R₁₅, an optionally substituted aryl, an optionally substituted heteroaryl, an optionally substituted heterocyclyl and an optionally substituted cycloalkyl;

R₈ is selected from hydrogen, F, Cl, Br, CN, an optionally substituted C₁-C₄ alkyl, an optionally substituted C₂-C₄ alkenyl, an optionally substituted C₂-C₄ alkynyl, an optionally substituted C₁-C₄ haloalkyl, an optionally substituted C₁-C₄ heteroalkyl, —OR₁₆, an optionally substituted aryl, an optionally substituted heteroaryl, an optionally substituted heterocyclyl and an optionally substituted cycloalkyl;

R₉ is selected from hydrogen, F, Cl, Br, CN, an optionally substituted C₁-C₄ alkyl, an optionally substituted C₂-C₄ alkenyl, an optionally substituted C₂-C₄ alkynyl, an optionally substituted C₁-C₄ haloalkyl, and an optionally substituted C₁-C₄ heteroalkyl, or

R₇ and R₈ together form an optionally substituted 5-6 member ring and R₉ is selected from hydrogen, F, Cl, Br, CN, an optionally substituted C₁-C₄ alkyl, an optionally substituted C₂-C₄ alkenyl, an optionally substituted C₂-C₄ alkynyl, an optionally substituted C₁-C₄ haloalkyl, an optionally substituted C₁-C₄ heteroalkyl, or

R₈ and R₉ together form an optionally substituted 4-6 member ring and R₇ is selected from hydrogen, F, Cl, Br, CN, an optionally substituted C₁-C₄ alkyl, an optionally substituted C₂-C₄ alkenyl, an optionally substituted C₂-C₄ alkynyl, an optionally substituted C₁-C₄ haloalkyl, an optionally substituted C₁-C₄ heteroalkyl, —CONR₁₄R₁₅, and an optionally substituted aryl;

R₁₀ is selected from hydrogen, F, Cl, Br, an optionally substituted C₂-C₄ alkyl, an optionally substituted C₂-C₄ alkenyl, an optionally substituted C₂-C₄ alkynyl; and

R₁₁ is selected from hydrogen, F, Cl, Br, CN, an optionally substituted C₁-C₄ alkyl, an optionally substituted C₂-C₄ alkenyl, an optionally substituted C₂-C₄ alkynyl, an optionally substituted C₁-C₄ haloalkyl, an optionally substituted C₁-C₄ heteroalkyl, hydroxyiminoalkyl, alkoxyiminoalkyl, aryloxyiminoalkyl, —CONR₁₄R₁₅, —OR₁₆, —COR₁₆, an optionally substituted aryl, an optionally substituted heteroaryl, an optionally substituted heterocyclyl and an optionally substituted cycloalkyl;

R₁₂ is selected from hydrogen, F, Cl, Br, CN, an optionally substituted C₁-C₄ alkyl, an optionally substituted C₂-C₄ alkenyl, an optionally substituted C₂-C₄ alkynyl, an optionally substituted C₁-C₄ haloalkyl, an optionally substituted C₁-C₄ heteroalkyl, —OR₁₆, an optionally substituted aryl, an optionally substituted heteroaryl, an optionally substituted heterocyclyl and an optionally substituted cycloalkyl;

R₁₃ is selected from hydrogen, F, Cl, Br, CN, CONR₁₄R₁₅, an optionally substituted C₁-C₄ alkyl, an optionally substituted C₂-C₄ alkenyl, an optionally substituted C₂-C₄ alkynyl, an optionally substituted C₁-C₄ haloalkyl, and an optionally substituted C₁-C₄ heteroalkyl, or

R₁₁ and R₁₂ together form an optionally substituted 5-6 member ring and R₁₃ is selected from hydrogen, F, Cl, Br, CN, CONR₁₄R₁₅, an optionally substituted C₁-C₄ alkyl, an optionally substituted C₂-C₄ alkenyl, an optionally substituted C₂-C₄ alkynyl, an optionally substituted C₁-C₄ haloalkyl, and an optionally substituted C₁-C₄ heteroalkyl, or

R₁₂ and R₁₃ together form an optionally substituted 4-6 member ring and R₁₁, is selected from hydrogen, F, Cl, Br, CN, an optionally substituted C₁-C₄ alkyl, an optionally substituted C₂-C₄ alkenyl, an optionally substituted C₂-C₄ alkynyl, an optionally substituted C₁-C₄ haloalkyl, an optionally substituted C₁-C₄ heteroalkyl, —CONR₁₄R₁₅, an optionally substituted aryl, an optionally substituted heteroaryl, an optionally substituted heterocyclyl and an optionally substituted cycloalkyl;

R₁₄ and R₁₅ are each independently selected from hydrogen, an optionally substituted C₁-C₄ alkyl, an optionally substituted C₂-C₄ alkenyl, an optionally substituted C₂-C₄ alkynyl, an optionally substituted C₁-C₄ haloalkyl, an optionally substituted aryl, an optionally substituted heteroaryl, an optionally substituted heterocyclyl, an optionally substituted cycloalkyl and an optionally substituted C₁-C₄ heteroalkyl, or

R₁₄ and R₁₅ together form an optionally substituted 4-7 member ring;

R₁₆ is selected from hydrogen, an optionally substituted C₁-C₄ alkyl, an optionally substituted C₂-C₄ alkenyl, an optionally substituted C₂-C₄ alkynyl, an optionally substituted C₁-C₄ haloalkyl, an optionally substituted C₁-C₄ heteroalkyl, an optionally substituted aryl, an optionally substituted heteroaryl, an optionally substituted heterocyclyl and an optionally substituted cycloalkyl;

X is selected from O, S, and NR₁₇; and

R₁₇ is selected from hydrogen and an optionally substituted C₁-C₄ alkyl, an optionally substituted C₂-C₄ alkenyl and an optionally substituted C₂-C₄ alkynyl;

wherein the substituents on the alkyl, alkenyl, alkynyl, aralkyl, aryl, heteroaryl, heterocyclyl, and cycloalkyl groups, when present, are each individually and independently selected from one or more group(s) selected from: alkyl, alkenyl, alkynyl, cycloalkyl, aryl, heteroaryl, non-aromatic heterocycle, hydroxy, alkoxy, alkoxyalkoxy, aryloxy, mercapto, alkylthio, arylthio, cyano, halo, carbonyl, imino, hydroxyimino, alkoxyimino, aryloxyimino, aralkoxyiminothiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy and amino,

wherein at least one position selected from R₂, R₃, R₄, R₅, and R₆ is not hydrogen;

at least one position selected from R₇, R₈, R₉, and R₁₀ is not hydrogen;

if R₄ is F, then at least one position selected from R₂, R₃, R₅ and R₆ is not hydrogen;

if R₃ is F, then at least one position selected from R₂, R₄, R₅, and R₆ is not hydrogen; and

if any two positions selected from R₂, R₃, R₄, R₅, and R₆ are both F, then at least one of the other three positions selected from R₂, R₃, R₄, R₅, and R₆ is not hydrogen.

In certain embodiments, the compounds provided are of Formula I:

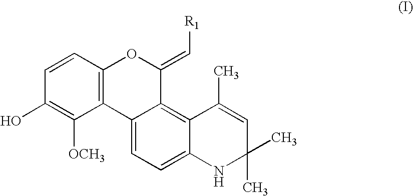

or a pharmaceutically acceptable salt, ester, amide, or prodrug thereof, wherein R₁ is selected from Formula II, III, and IV:

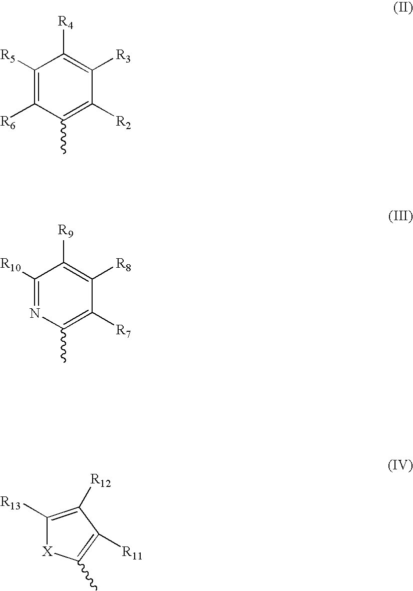

wherein:

R₂ is selected from hydrogen, F, Cl, Br, CN, an optionally substituted C₁-C₄ alkyl, an optionally substituted C₁-C₄ haloalkyl, an optionally substituted C₁-C₄ heteroalkyl, —CONR₁₄R₁₅, —OR₁₆, —SR₁₆, —SO₂NR₁₄R₁₅, and an optionally substituted aryl,

R₃ is selected from hydrogen, F, Cl, Br, CN, an optionally substituted C₁-C₄ alkyl, an optionally substituted C₁-C₄ haloalkyl, an optionally substituted C₁-C₄ heteroalkyl, —OR₁₆, —SR₁₆ and an optionally substituted aryl, and

R₄ is selected from hydrogen, F, Cl, Br, CN, —OR₁₆, a ring, an optionally substituted C₁-C₄ alkyl, an optionally substituted C₁-C₄ haloalkyl, and an optionally substituted C₁-C₄ heteroalkyl, or

R₂ and R₃ together form an optionally substituted 5-6 member ring and R₄ is selected from hydrogen, F, Cl, Br, CN, —OR₁₆, a ring, an optionally substituted C₁-C₄ alkyl, an optionally substituted C₁-C₄ haloalkyl, and an optionally substituted C₁-C₄ heteroalkyl, or

R₃ and R₄ together form an optionally substituted 4-6 member ring and R₂ is selected from hydrogen, F, Cl, Br, CN, an optionally substituted C₁-C₄ alkyl, an optionally substituted C₁-C₄ haloalkyl, an optionally substituted C₁-C₄ heteroalkyl, —CONR₁₄R₁₅, —OR₁₆, —SR₁₆, —SO₂NR₁₄R₁₅, and an optionally substituted aryl;

R₅ is selected from hydrogen, F, Cl, Br, optionally substituted C₁-C₄ alkyl, and OCH₃;

R₆ is selected from hydrogen and F;

R₇ is selected from hydrogen, F, Cl, Br, CN, an optionally substituted C₁-C₄ alkyl, an optionally substituted C₁-C₄ haloalkyl, an optionally substituted C₁-C₄ heteroalkyl, —CONR₁₄R₁₅, and an optionally substituted aryl,

R₈ is selected from hydrogen, F, Cl, Br, CN, an optionally substituted C₁-C₄ alkyl, an optionally substituted C₁-C₄ haloalkyl, an optionally substituted C₁-C₄ heteroalkyl, —OR₁₆, a phenyl that is optionally substituted with hydrogen, a halogen, an optionally substituted C₁-C₄ alkyl, an optionally substituted C₁-C₄ haloalkyl, and an optionally substituted C₁-C₄ heteroalkyl, and

R₉ is selected from hydrogen, F, Cl, Br, CN, an optionally substituted C₁-C₄ alkyl, an optionally substituted C₁-C₄ haloalkyl, and an optionally substituted C₁-C₄ heteroalkyl, or

R₇ and R₈ together form an optionally substituted 5-6 member ring and R₉ is selected from hydrogen, F, Cl, Br, CN, an optionally substituted C₁-C₄ alkyl, an optionally substituted C₁-C₄ haloalkyl, an optionally substituted C₁-C₄ heteroalkyl, or

R₈ and R₉ together form an optionally substituted 4-6 member ring and R₇ is selected from hydrogen, F, Cl, Br, CN, an optionally substituted C₁-C₄ alkyl, an optionally substituted C₁-C₄ haloalkyl, an optionally substituted C₁-C₄ heteroalkyl, —CONR₁₄R₁ₛ, and an optionally substituted aryl;

R₁₀ is selected from hydrogen, F, Cl, CH₃, and OCH₃;

R₁₁ is selected from hydrogen, F, Cl, Br, CN, an optionally substituted C₁-C₄ alkyl, an optionally substituted C₁-C₄ haloalkyl, an optionally substituted C₁-C₄ heteroalkyl, —CONR₁₄R₁₅, and an optionally substituted aryl,

R₁₂ is selected from hydrogen, F, Cl, Br, CN, an optionally substituted C₁-C₄ alkyl, an optionally substituted C₁-C₄ haloalkyl, an optionally substituted C₁-C₄ heteroalkyl, —OR₁₆, a phenyl that is optionally substituted with hydrogen, a halogen, an optionally substituted C₁-C₄ alkyl, an optionally substituted C₁-C₄ haloalkyl, and an optionally substituted C₁-C₄ heteroalkyl, and

R₁₃ is selected from hydrogen, F, Cl, Br, CN, CONR₁₄R₁₅, an optionally substituted C₁-C₄ alkyl, an optionally substituted C₁-C₄ haloalkyl, and an optionally substituted C₁-C₄ heteroalkyl, or

R₁₁ and R₁₂ together form an optionally substituted 5-6 member ring and R₁₃ is selected from hydrogen, F, Cl, Br, CN, CONR₁₄R₁₅, all optionally substituted C₁-C₄ alkyl, an optionally substituted C₁-C₄ haloalkyl, and an optionally substituted C₁-C₄ heteroalkyl, or

R₁₂ and R₁₃ together form an optionally substituted 4-6 member ring and R₁₁ is selected from hydrogen, F, Cl, Br, CN, an optionally substituted C₁-C₄ alkyl, an optionally substituted C₁-C₄ haloalkyl, an optionally substituted C₁-C₄ heteroalkyl, —CONR₁₄R₁₅, and an optionally substituted aryl;

R₁₄ and R₁ₛ are each independently selected from hydrogen, an optionally substituted C₁-C₄ alkyl, an optionally substituted C₁-C₄ haloalkyl, and an optionally substituted C₁-C₄ heteroalkyl, or

R₁₄ and R₁₅ together form an optionally substituted 4-7 member ring;

R₁₆ is selected from hydrogen, an optionally substituted C₁-C₄ alkyl, an optionally substituted C₁-C₄ haloalkyl, an optionally substituted C₁-C₄ heteroalkyl, and an optionally substituted aryl;

X is selected from O, S, and NR₁₇; and

R₁₇ is selected from hydrogen and an optionally substituted C₁-C₄ alkyl;

wherein

at least one position selected from R₂, R₃, R₄, R₅, and R₆ is not hydrogen;

at least one position selected from R₇, R₈, R₉, and R₁₀ is not hydrogen;

if R₄ is F, then at least one position selected from R₂, R₃, R₅ and R₆ is not hydrogen;

if R₃ is F, then at least one position selected from R₂, R₄, R₅, and R₆ is not hydrogen; and

if any two positions selected from R₂, R₃, R₄, R₅, and R₆ are both F, then at least one of the other three positions selected from R₂, R₃, R₄, R₅, and R₆ is not hydrogen.

a. R₁ has Formula II

In certain embodiments, the compounds provided herein are of formula I, wherein R₁ has Formula II. In certain embodiments, R₂ is selected from hydrogen, halo, cyano, C₁-C₄ alkyl, C₂-C₄ alkenyl, aryl, haloalkoxy, haloalkylthio, formylaryl, hydroxyC₁-C₄alkyl, diC₁-C₄alkylaminoC₁-C₄alkyl, C₁-C₄alkylcarbonyl, hydroxyiminoC₁-C₄alkyl, alkoxyiminoC₁-C₄alkyl, alkoxyalkoxyC₁-C₄alkyl, hydroxyhaloC₂-C₄ alkyl, hydroxyhaloC₂-C₄ alkenyl, C₁-C₄alkylcarbonyloxyC₁-C₄alkyl, formyl, —OR₁₆, —SR₁₆, —CONR₁₄R₁₅, —SO₂NR₁₄R₁₅, wherein R₁₄ and R₁₅ are each independently selected from hydrogen, C₁-C₄ alkyl, C₅-C₆ aryl C₁-C₄alkyl, C₃-C₇ cycloalkyl, or R₁₄ and R₁₅ together form an optionally substituted 4-7 member ring containing 1 or 2 heteroatoms selected from nitrogen and oxygen.

In certain embodiments, R₂ is selected from halo, cyano, C₁-C₄ alkyl, C₂-C₄ alkenyl, aryl, haloalkoxy, haloalkylthio, formylaryl, hydroxyC₁-C₄alkyl, diC₁-C₄alkylaminoC₁-C₄alkyl, C₁-C₄alkylcarbonyl, hydroxyiminoC₁-C₄alkyl, alkoxyiminoC₁-C₄alkyl, alkoxyalkoxyC₁-C₄alkyl, hydroxyhaloC₂-C₄ alkyl, hydroxyhaloC₂-C₄ alkenyl, C₁-C₄alkylcarbonyloxyC₁-C₄alkyl, formyl, —OR₁₆, —SR₁₆, —CONR₁₄R₁₅, —SO₂NR₁₄R₁₅, wherein R₁₄ and R₁₅ are each independently selected from hydrogen, C₁-C₄ alkyl, C₅-C₆ aryl C₁-C₄alkyl, C₃-C₇ cycloalkyl, or R₁₄ and R₁₅ together form an optionally substituted 4-7 member ring containing 1 or 2 heteroatoms selected from nitrogen and oxygen.

In certain embodiments, R₂ is selected from hydrogen, F, Cl, Br, CN, an optionally substituted C₁-C₄ alkyl, an optionally substituted C₁-C₄ haloalkyl, an optionally substituted C₁-C₄ heteroalkyl, —CONR₁₄R₁₅, —OR₁₆, —SR₁₆, —SO₂NR₁₄R₁₅, and an optionally substituted aryl. In certain embodiments in which R₂ is an optionally substituted aryl, R₂ is an optionally substituted phenyl. In certain embodiments, R₂ is an optionally substituted phenyl that is optionally substituted with a substituent selected from hydrogen, a halogen, an optionally substituted C₁-C₄ alkyl, an optionally substituted C₁-C₄ haloalkyl, and an optionally substituted C₁-C₄ heteroalkyl.

In certain embodiments, R₂ is selected from hydrogen, halo, cyano, C₁-C₄ alkyl, C₂-C₄ alkenyl, haloalkoxy, hydroxyC₁-C₄alkyl, alkoxyalkoxyC₁-C₄ alkyl, and hydroxyhaloC₁-C₄ alkyl.

In certain embodiments, R₂ is selected from halo, cyano, C₁-C₄ alkyl, C₂-C₄ alkenyl, haloalkoxy, hydroxyC₁-C₄alkyl, alkoxyalkoxyC₁-C₄ alkyl, and hydroxyhaloC₁-C₄ alkyl.

In certain embodiments, R₂ is selected from hydrogen, fluoro, chloro, bromo, cyano, methyl, vinyl, hydroxymethyl, diethylaminomethyl, methoxymethoxymethyl, hydroxyiminomethyl, acetyloxymethyl, 1-hydroxy-2,2,2-trifluoroethyl, phenyl, trifluoromethoxy, trifluoromethylthio, acetyl, formyl, diethylaminocarbonyl, 3-formylphenyl, N-benzyl-N-methylaminocarbonyl, dimethylaminocarbonyl, 1-pyrrolidinocarbonyl, 1-morpholinocarbonyl, 4-methyl piperazi-1-nocarbonyl, piperidinocarbonyl, N-cyclohexyl-N-methylaminocarbonyl, piperidinosulfonyl, and N,N-dimethylaminosulfonyl.

In certain embodiments, R₂ is selected from fluoro, chloro, bromo, cyano, methyl, vinyl, hydroxymethyl, diethylaminomethyl, methoxymethoxymethyl, hydroxyiminomethyl, acetyloxymethyl, 1-hydroxy-2,2,2-trifluoroethyl, phenyl, trifluoromethoxy, trifluoromethylthio, acetyl, formyl, diethylaminocarbonyl, 3-formylphenyl, N-benzyl-N-methylaminocarbonyl, dimethylaminocarbonyl, 1-pyrrolidinocarbonyl, 1-morpholinocarbonyl, 4-methyl piperazi-1-nocarbonyl, piperidinocarbonyl, N-cyclohexyl-N-methylaminocarbonyl, piperidinosulfonyl, and N,N-dimethylaminosulfonyl.

In certain embodiments, R₂ is selected from hydrogen, fluoro, chloro, cyano, methyl, hydroxymethyl, methoxymethoxymethyl, 1-hydroxy-2,2,2-trifluoroethyl, vinyl and trifluoromethoxy.

In certain embodiments, R₃ is selected from hydrogen, halo, hydroxy, C₁-C₄alkoxy, C₁-C₄alkyl, haloC₁-C₄alkyl, haloalkoxy, haloC₁-C₄alkylthio, aryl, heteroaryl, haloaryloxy, aryloxy, haloaryloxy, alkoxyaryloxy, C₁-C₄alkylaryloxy, haloalkoxyaryloxy, haloaryl and hydroxyC₁-C₄alkyl.

In certain embodiments, R₃ is selected from halo, hydroxy, C₁-C₄alkoxy, C₁-C₄alkyl, haloC₁-C₄alkyl, haloalkoxy, haloC₁-C₄alkylthio, aryl, heteroaryl, haloaryloxy, aryloxy, haloaryloxy, alkoxyaryloxy, C₁-C₄alkylaryloxy, haloalkoxyaryloxy, haloaryl and hydroxyC₁-C₄alkyl.

In certain embodiments, R₃ is selected from hydrogen, F, Cl, Br, CN, an optionally substituted C₁-C₄ alkyl, an optionally substituted C₁-C₄ haloalkyl, an optionally substituted C₁-C₄ heteroalkyl, —OR₁₆, —SR₁₆ and an optionally substituted aryl. In certain embodiments in which R₃ is an optionally substituted aryl, R₃ is an optionally substituted phenyl. In certain of such embodiments, R₃ is an optionally substituted phenyl that is optionally substituted with a substituent selected from hydrogen, a halogen, an optionally substituted C₁-C₄ alkyl, an optionally substituted C₁-C₄ haloalkyl, and an optionally substituted C₁-C₄ heteroalkyl.

In certain embodiments, R₃ is selected from hydrogen, halo, hydroxy, C₁-C₄alkoxy, C₁-C₄alkyl, haloC₁-C₄alkyl, haloalkoxy, haloC₁-C₄alkylthio, haloaryloxy, and aryloxy.

In certain embodiments, R₃ is selected from halo, hydroxy, C₁-C₄alkoxy, C₁-C₄alkyl, haloC₁-C₄alkyl, haloalkoxy, haloC₁-C₄alkylthio, haloaryloxy, and aryloxy.

In certain embodiments, R₃ is selected from hydrogen, fluoro, chloro, bromo, hydroxy, methoxy, methyl, tert-butyl, trifluoromethyl, hydroxymethyl, trifluoromethoxy, trifluoromethylthio, phenyl, 2,2-difluoro-1-ethoxy, 4,4,4-trifluoro-but-1-oxy, 2,4-difluorophenyl, 2-fluorophenyl, phenoxy, 3,6-dichlorophenoxy, 4-methoxyphenoxy, 3,4-dichlorophenoxy, 4-methylphenoxy, 4-chlorophenoxy, 3-trifluoromethoxyphenoxy, 4-fluorophenoxy, 3-thienyl, 2,2-difluoro-3,3,3-trifluoroprop-1-yloxy, 3,5-dichlorophenoxy, 4-fluorobenzyloxy, 3-fluorobenzyloxy and 3-pyridyl.

In certain embodiments, R₃ is selected from fluoro, chloro, bromo, hydroxy, methoxy, methyl, tert-butyl, trifluoromethyl, hydroxymethyl, trifluoromethoxy, trifluoromethylthio, phenyl, 2,2-difluoro-1-ethoxy, 4,4,4-trifluoro-but-1-oxy, 2,4-difluorophenyl, 2-fluorophenyl, phenoxy, 3,6-dichlorophenoxy, 4-methoxyphenoxy, 3,4-dichlorophenoxy, 4-methylphenoxy, 4-chlorophenoxy, 3-trifluoromethoxyphenoxy, 4-fluorophenoxy, 3-thienyl, 2,2-difluoro-3,3,3-trifluoroprop-1-yloxy, 3,5-dichlorophenoxy, 4-fluorobenzyloxy, 3-fluorobenzyloxy and 3-pyridyl.

In certain embodiments, R₃ is selected from hydrogen, fluoro, chloro, hydroxy, methyl, trifluoromethyl, hydroxymethyl, trifluoromethoxy, phenoxy, trifluoromethylthio and 4-fluorophenoxy.

In certain embodiments, R₃ is selected from fluoro, chloro, hydroxy, methyl, trifluoromethyl, hydroxymethyl, trifluoromethoxy, phenoxy, trifluoromethylthio and 4-fluorophenoxy.

In certain embodiments, R₄ is selected from hydrogen, halo, hydroxy, C₁-C₄ alkyl, C₂-C₄alkenyl, C₃-C₆cycloalkyl, haloC₁-C₄ alkyl, aryl, hydroxy C₁-C₄alkyl, alkoxy, haloalkoxy, aralkoxy, haloaralkoxy, alkylaralkoxy, haloaryl, or R₃ and R₄ together form alkelenedioxy.

In certain embodiments, R₄ is selected from halo, hydroxy, C₁-C₄ alkyl, C₂-C₄alkenyl, C₃-C₆cycloalkyl, haloC₁-C₄ alkyl, aryl, hydroxy C₁-C₄alkyl, alkoxy, haloalkoxy, aralkoxy, haloaralkoxy, alkylaralkoxy, haloaryl, or R₃ and R₄ together form alkelenedioxy.

In certain embodiments, R₄ is selected from hydrogen, F, Cl, Br, CN, —OR₁₆, a ring, an optionally substituted C₁-C₄ alkyl, an optionally substituted C₁-C₄ haloalkyl, and an optionally substituted C₁-C₄ heteroalkyl. In certain embodiments, R₄ is selected from hydrogen and halogen. In certain embodiments, R₄ is halogen. In certain embodiments, R₄ is hydrogen.

In certain embodiments, R₄ is selected from hydrogen, chloro, bromo, hydroxy, methoxy, fluoro, trifluoromethoxy, methyl, ethyl, isopropyl, vinyl, benzyloxy, phenyl, cyclohexyl, trifluoromethyl, 4-methylbenzyloxy, hydroxymethyl, or R₃ and R₄ together form an methelenedioxy. In certain embodiments, R₄ is F.

In certain embodiments, R₄ is selected from chloro, bromo, hydroxy, methoxy, fluoro, trifluoromethoxy, methyl, ethyl, isopropyl, vinyl, benzyloxy, phenyl, cyclohexyl, trifluoromethyl, 4-methylbenzyloxy, hydroxymethyl, or R₃ and R₄ together form an methelenedioxy.

In certain embodiments, R₂ and R₃ together form an optionally substituted 5-6 member ring and R₄ is selected from hydrogen, F, Cl, Br, CN, —OR₁₆, a ring, —SO₂NR₁₄R₁₅, an optionally substituted C₁-C₄ alkyl, an optionally substituted C₁-C₄ haloalkyl, and an optionally substituted C₁-C₄ heteroalkyl. In certain embodiments, R₃ and R₄ together form an optionally substituted 4-6 member ring and R₂ is selected from hydrogen, F, Cl, Br, CN, an optionally substituted C₁-C₄ alkyl, an optionally substituted C₁-C₄ haloalkyl, an optionally substituted C₁-C₄ heteroalkyl, —CONR₁₄R₁₅, —OR₆, —SR₆, —SO₂NR₁₄R₁₅, and an optionally substituted aryl.

In certain embodiments, R₂ and R₃ together form alkelenedioxy. In certain embodiments, R₂ and R₃ together with the phenyl ring on which they are substituted form optionally substituted benzo-1,3-dioxan or optionally substituted naphthyl ring.

In certain embodiments, R₅ is selected from hydrogen, halo, halo C₁-C₄alkyl, C₁-C₄alkyl, and C₁-C₄alkoxy. In certain embodiments, R₅ is selected from hydrogen, F, Cl, Br, optionally substituted C₁-C₄ alkyl, and OCH₃. In certain embodiments, R₅ is selected from hydrogen, chloro, fluoro, bromo, methyl, trifluoromethyl, isobutyl and methoxy. In certain embodiments, R₅ is selected from halo, halo C₁-C₄alkyl, C₁-C₄alkyl, and C₁-C₄alkoxy. In certain embodiments, R₅ is selected from F, Cl, Br, optionally substituted C₁-C₄ alkyl, and OCH₃. In certain embodiments, R₅ is selected from chloro, fluoro, bromo, methyl, trifluoromethyl, isobutyl and methoxy. In certain embodiments, R₅ is selected from hydrogen and halogen. In certain embodiments, R₅ is halogen. In certain embodiments, R₅ is hydrogen. In certain embodiments, R₅ is fluoro.

In certain embodiments, R₆ is selected from hydrogen, halo and C₁-C₄alkyl. In certain embodiments, R₆ is selected from halo and C₁-C₄alkyl. In certain embodiments, R₆ is selected from hydrogen and halo. In certain embodiments, R₆ is selected from hydrogen and fluoro. In certain embodiments, R₆ is hydrogen. In certain embodiments, R₆ is fluoro.

In certain embodiments, at least one position selected from R₂, R₃, R₄, R₅, and R₆ is not hydrogen. In certain embodiments, at least one position selected from R₇, R₈, R₉, and R₁₀ is not hydrogen. In certain embodiments, if R₄ is F, then at least one position selected from R₂, R₃, R₅ and R₆ is not hydrogen. In certain embodiments, if R₃ is F, then at least one position selected from R₂, R₄, R₅, and R₆ is not hydrogen. In certain embodiments, if any two positions selected from R₂, R₃, R₄, R₅, and R₆ are both F, then at least one of the other three positions selected from R₂, R₃, R₄, R₅, and R₆ is not hydrogen.

In certain embodiments, R₁ is:

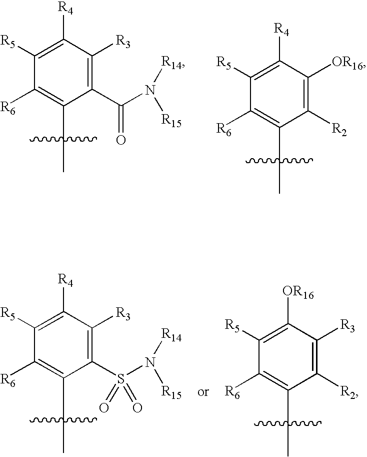

wherein the variables are as described elsewhere herein.

In certain embodiments, R₁ is

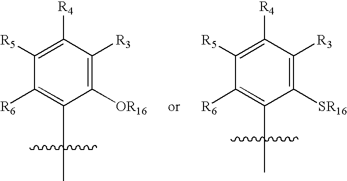

wherein the variables are as described elsewhere herein.

In certain embodiments, R₁₆ is hydrogen, optionally substituted C₁-C₄alkyl, haloC₁-C₄alkyl, optionally substituted aryl, haloaryloxy and C₁-C₄alkoxyC₁-C₄alkyl; and the other variables are as described elsewhere herein. In certain embodiments, R₁₆ is optionally substituted C₁-C₄alkyl, haloC₁-C₄alkyl, optionally substituted aryl, haloaryloxy and C₁-C₄alkoxyC₁-C₄alkyl; and the other variables are as described elsewhere herein.

In certain embodiments, R₁ is

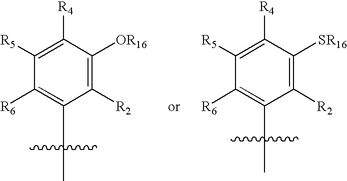

wherein the variables are as described elsewhere herein.

In certain embodiments, R₁₆ is hydrogen, methyl, methoxy, trifluoromethyl, 4-fluorophenyl, 4-methylbenzyl, 4,4,4-trifluorobutyl, 2-fluoroethyl, 2,2-difluoro-3,3,3-trifluoropropyl, 4-fluorobenzyl, 2-fluorobenzyl, 4-methoxyphenyl, 3,4-dichlorophenyl, 4-tolyl, 4-chlorophenyl, 3-trifluoromethoxyphenyl, and phenyl.

In certain embodiments, R₁ is

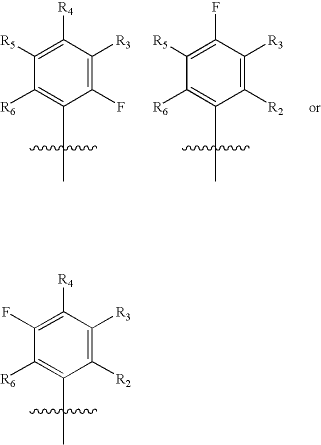

wherein the variables are as described elsewhere herein.

In certain embodiments, at least one position selected from R₂, R₃, R₄, R₅, and R₆ is not hydrogen. In certain embodiments, at least one position selected from R₇, R₈, R₉, and R₁₀ is not hydrogen. In certain embodiments, if R₄ is F, then at least one position selected from R₂, R₃, R₅ and R₆ is not hydrogen. In certain embodiments, if R₃ is F, then at least one position selected from R₂, R₄, R₅, and R₆ is not hydrogen. In certain embodiments, if any two positions selected from R₂, R₃, R₄, R₅, and R₆ are both F, then at least one of the other three positions selected from R₂, R₃, R₄, R₅, and R₆ is not hydrogen.

b. R₁ has Formula III

In certain embodiments, the compounds provided herein are of Formula I, wherein R₁ has Formula III. In certain embodiments, R₇ is selected from hydrogen, F, Cl, Br, CN, an optionally substituted C₁-C₄ alkyl, an optionally substituted C₁-C₄ haloalkyl, an optionally substituted C₁-C₄ heteroalkyl, —CONR₁₄R₁₅, and an optionally substituted aryl. In certain embodiments, R₇ is selected from F, Cl, Br, CN, an optionally substituted C₁-C₄ alkyl, an optionally substituted C₁-C₄ haloalkyl, an optionally substituted C₁-C₄ heteroalkyl, —CONR₁₄R₁ₛ, and an optionally substituted aryl. In certain embodiments in which R₇ is an optionally substituted aryl, R₇ is an optionally substituted phenyl. In certain of such embodiments, R₇ is an optionally substituted phenyl that is optionally substituted with a substituent selected from hydrogen, a halogen, an optionally substituted C₁-C₄ alkyl, an optionally substituted C₁-C₄ haloalkyl, and an optionally substituted C₁-C₄ heteroalkyl. In certain embodiments, R₇ is hydrogen or hydroxyalkyl. In certain embodiments, R₇ is hydrogen or hydroxymethyl. In certain embodiments, R₇ is hydrogen.

In certain embodiments, R₈ is selected from hydrogen, F, Cl, Br, CN, an optionally substituted C₁-C₄ alkyl, an optionally substituted C₁-C₄ haloalkyl, an optionally substituted C₁-C₄ heteroalkyl, —OR₁₆, a phenyl that is optionally substituted with hydrogen, a halogen, an optionally substituted C₁-C₄ alkyl, an optionally substituted C₁-C₄ haloalkyl, and an optionally substituted C₁-C₄ heteroalkyl. In certain embodiments, R₈ is selected from F, Cl, Br, CN, an optionally substituted C₁-C₄ alkyl, an optionally substituted C₁-C₄ haloalkyl, an optionally substituted C₁-C₄ heteroalkyl, —OR₁₆, a phenyl that is optionally substituted with hydrogen, a halogen, an optionally substituted C₁-C₄ alkyl, an optionally substituted C₁-C₄ haloalkyl, and an optionally substituted C₁-C₄ heteroalkyl. In certain embodiments, R₈ is hydrogen or alkyl. In certain embodiments, R₈ is hydrogen. In certain embodiments, R₈ is alkyl.

In certain embodiments, R₉ is selected from hydrogen, F, Cl, Br, CN, an optionally substituted C₁-C₄ alkyl, an optionally substituted C₁-C₄ haloalkyl, and an optionally substituted C₁-C₄ heteroalkyl. In certain embodiments, R₉ is selected from F, Cl, Br, CN, an optionally substituted C₁-C₄ alkyl, an optionally substituted C₁-C₄ haloalkyl, and an optionally substituted C₁-C₄ heteroalkyl.

In certain embodiments, R₇ and R₈ together form an optionally substituted 5-6 member ring and R₉ is selected from hydrogen, F, Cl, Br, CN, an optionally substituted C₁-C₄ alkyl, an optionally substituted C₁-C₄ haloalkyl, an optionally substituted C₁-C₄ heteroalkyl. In certain embodiments, R₈ and R₉ together form an optionally substituted 4-6 member ring and R₇ is selected from hydrogen, F, Cl, Br, CN, an optionally substituted C₁-C₄ alkyl, an optionally substituted C₁-C₄ haloalkyl, an optionally substituted C₁-C₄ heteroalkyl, —CONR₁₄R₁₅, and an optionally substituted aryl.

In certain embodiments, R₁₀ is selected from hydrogen, F, Cl, Br, alkyl and alkoxy. In certain embodiments, R₁₀ is selected from F, Cl, Br, alkyl and alkoxy. In certain embodiments, R₁₀ is selected from hydrogen, F, Cl, CH₃, and OCH₃. In certain embodiments, R₁₀ is hydrogen or CH₃. In certain embodiments, R₁₀ is selected from hydrogen, CH₃, and OCH₃. In certain embodiments, R₁₀ is hydrogen.

In certain embodiments, R₁ is:

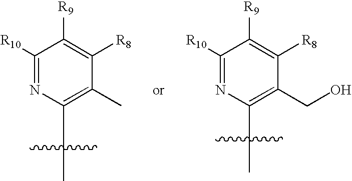

wherein the variables are as described elsewhere herein.

c. R₁ has Formula IV

In certain embodiments, the compounds provided herein are of formula I,

wherein R₁ has Formula IV. In certain embodiments, R₁₁ is selected from hydrogen, cyano, formyl, C₁-C₄alkyl, C₂-C₄alkenyl, C₂-C₄alkynyl, hydroxyC₁-C₄alkyl, haloC₁-C₄alkyl, haloC₂-C₄alkenyl, hydroxyC₁-C₄alkyl, hydroxyC₂-C₄alkenyl, cyanoC₁-C₄alkenyl, hydroxyC₂-C₄alkynyl, alkoxyalkoxyC₁-C₄alkyl, hydroxyhaloC₁-C₄alkyl, aminoC₁-C₄alkyl, C₁-C₄alkylaminoC₁-C₄alkyl, diC₁-C₄alkylaminoC₁-C₄alkyl, C₁-C₄alkylC₂-C₄alkenylaminoC₁-C₄alkyl, arylaminoC₁-C₄alkyl, C₂-C₄alkenylaminoC₁-C₄alkyl, cycloC₃-C₆alkylaminoC₁-C₄alkyl, hydroxyalkoxyalkyl, haloalkylcarbonyl, alkoxyalkoxyalkoxy, carboxyalkoxyalkyl, alkoxyhaloalkyl, alkoxycarbonylalkenyl, hydroxy C₁-C₄alkylcarbamoyl, N,N-diC₁-C₄alkylaminoC₁-C₄alkyl, N-cycloC₃-C₆alkyl-N—C₁-C₄alkylaminocarbonyl, haloC₁-C₄alkylcarbamoyl, hydroxyhaloC₁-C₄alkyl, C₁-C₄alkylcarbonyl, cycloC₃-C₆alkylcarbonyl, C₂-C₄alkenylcarbonyl, C₂-C₄alkynylcarbonyl, arylcarbonyl, heteroarylcarbonyl, hydroxyaralkyl, C₁-C₄alkoxyC₁-C₄alkyl, C₂-C₄alkenyloxyC₁-C₄alkyl, C₂-C₄alkynyloxyC₁-C₄alkyl, aryloxyC₁-C₄alkyl, hydroxyiminoC₁-C₄alkyl, alkoxyiminoC₁-C₄alkyl, C₂-C₄alkenyloxyiiminoC₁-C₄alkyl, aryloxyiminoC₁-C₄ alkyl, aralkoxyiminoC₁-C₄alkyl, heterocyclyl, heteroaryl and CONR₁₄R₁₅, wherein the alkyl, alkenyl, alkyl, cycloalkyl, heterocyclyl, heteroaryl and aryl groups can be unsubstituted or substituted with one to three substituents selected from C₁-C₄-alkyl, C₂-C₄alkenyl, C₂-C₄alkynyl, hydroxy, C₁-C₄alkoxy, nitro, halo, cyano, oxo, aryl, cycloalkyl, heterocyclyl, and heteroaryl groups.

In certain embodiments, R₁₁ is selected from cyano, formyl, C₁-C₄alkyl, C₂-C₄alkenyl, C₂-C₄alkynyl, hydroxyC₁-C₄alkyl, haloC₁-C₄alkyl, haloC₂-C₄alkenyl, hydroxyC₁-C₄alkyl, hydroxyC₂-C₄alkenyl, cyanoC₁-C₄alkenyl, hydroxyC₂-C₄alkynyl, alkoxyalkoxyC₁-C₄alkyl, hydroxyhaloC₁-C₄alkyl, aminoC₁-C₄alkyl, C₁-C₄alkylaminoC₁-C₄alkyl, diC₁-C₄alkylaminoC₁-C₄alkyl, C₁-C₄alkylC₂-C₄alkenylaminoC₁-C₄alkyl, arylaminoC₁-C₄alkyl, C₂-C₄alkenylaminoC₁-C₄alkyl, cycloC₃-C₆alkylaminoC₁-C₄alkyl, hydroxyalkoxyalkyl, haloalkylcarbonyl, alkoxyalkoxyalkoxy, carboxyalkoxyalkyl, alkoxyhaloalkyl, alkoxycarbonylalkenyl, hydroxy C₁-C₄alkylcarbamoyl, N,N-diC₁-C₄alkylaminoC₁-C₄alkyl, N-cycloC₃-C₆alkyl-N—C₁-C₄alkylaminocarbonyl, haloC₁-C₄alkylcarbamoyl, hydroxyhaloC₁-C₄alkyl, C₁-C₄alkylcarbonyl, cycloC₃-C₆alkylcarbonyl, C₂-C₄alkenylcarbonyl, C₂-C₄alkynylcarbonyl, arylcarbonyl, heteroarylcarbonyl, hydroxyaralkyl, C₁-C₄alkoxyC₁-C₄alkyl, C₂-C₄alkenyloxyC₁-C₄alkyl, C₂-C₄alkynyloxyC₁-C₄alkyl, aryloxyC₁-C₄alkyl, hydroxyiminoC₁-C₄alkyl, alkoxyiminoC₁-C₄alkyl, C₂-C₄alkenyloxyiminoC₁-C₄alkyl, aryloxyiminoC₁-C₄-alkyl, aralkoxyiminoC₁-C₄alkyl, heterocyclyl, heteroaryl and CONR₁₃R₁₄, wherein the alkyl, alkenyl, alkynyl, cycloalkyl, heterocyclyl, heteroaryl and aryl groups can be unsubstituted or substituted with one to three substituents selected from C₁-C₄-alkyl, C₂-C₄alkenyl, C₂-C₄alkynyl, hydroxy, C₁-C₄alkoxy, nitro, halo, cyano, oxo, aryl, cycloalkyl, heterocyclyl, and heteroaryl groups.

In certain embodiments, R₁₁ is selected from hydroxyC₁-C₄alkyl, hydroxyiminoC₁-C₄alkyl, C₁-C₄alkoxyiminoC₁-C₄alkyl, C₁-C₄alkylcarbonyl, C₁-C₄alkenyloxyiminoC₁-C₄alkyl, aryloxyiminoC₁-C₄alkyl, aralkoxyiminoC₁-C₄alkyl, C₁-C₄alkoxyC₁-C₄alkyl, C₁-C₄alkoxyalkoxyC₁-C₄alkyl, hydroxyhaloC₁-C₄alkyl, cycloalkylcarbonyl, C₂-C₄alkynylaminoC₁-C₄alkyl, haloC₁-C₄alkylaminoC₁-C₄alkyl, hydroxyalkoxyC₁-C₄alkyl, cyanoC₂-C₄alkenyl, alkoxyhaloC₁-C₄alkyl, heterocyclylcarbonyl and haloalkylcarbamoyl.

In certain embodiments, R₁₁ is selected from hydrogen, F, Cl, Br, CN, an optionally substituted C₁-C₄ alkyl, an optionally substituted C₁-C₄ haloalkyl, an optionally substituted C₁-C₄ heteroalkyl, —CONR₁₄R₁₅, and an optionally substituted aryl. In certain embodiments in which R₁₁ is an optionally substituted aryl, R₁₁ is an optionally substituted phenyl. In certain of such embodiments, R₁₁ is an optionally substituted phenyl that is optionally substituted with a substituent selected from hydrogen, a halogen, an optionally substituted C₁-C₄ alkyl, an optionally substituted C₁-C₄ haloalkyl, and an optionally substituted C₁-C₄ heteroalkyl.

In certain embodiments, R₁₁ is selected from hydrogen, cyano, carbamoyl, hydroxymethyl, 1-hydroxyethyl, vinyl, acetyl, 1-hydroxy-1-methylethyl, methoxymethyl, 4-fluorophenylhydroxymethyl, cyclohexylhydroxymethyl, hydroxythien-3-ylmethyl, hydroxythien-2-ylmethyl, N,N-diethylaminocarbonyl, methoxymethoxymethyl, 3-prop-2-enyloxymethyl, 1-hydroxybut-3-enyl, 1-hydroxy-2-phenylethyl, acroloyl, 4-fluorobenzoyl, thien-2-ylcarbonyl, cyclohexylcarbonyl, aminomethyl, phenylaminomethyl, prop-2-ynylaminomethyl, 2,2,2,-trifluoroethylaminomethyl, cyclopropylaminomethyl, butylaminomethyl, 2-hydroxyethoxymethyl, isopropenyl, formyl, trifluoroacetyl, methoxyethoxymethoxy, 2,2,2-trifluoro-1-hydroxy-1-(trifluoromethyl)ethyl, but-2-ynloxymethyl, 1-cyanovinyl, prop-3-ynyloxymethyl, 4-hydroxybut-3-enyl, 1-hydroxy-2-trifluoroethyl, ethoxycarbonylmethoxymethyl, carboxymethoxymethyl, 1-hydroxyprop-2-ynyl, 1-methoxy-2,2,2-trifluoroethyl, 2,2,2-trifluoro-1-methoxy-1-(trifluoromethyl)ethyl, 1-hydroxy-1-(thien-3-yl)ethyl, 2-methoxycarbonylvinyl, hydroxyethylcarbamoyl, ethylcarbamoyl, 2-(carbomethoxy)pyrrolidinocarbonyl, piperazinocarbonyl, N,N-dimethylaminomethyl, N,N-dimethylaminocarbonyl, N-ethyl-N-methylaminocarbonyl, N-morpholinocarbonyl, cyclopropyl, N-cyclohexyl-N-methylaminocarbonyl, 1-pyrrolidinocarbonyl, 2,2,2,-trifluoroethylcarbamoyl, 4-hydroxypiperidinecarbonyl, 4-methylpiperazinecarbonyl, 1-hydroxy-4,4,4-trifluorobut-2-ynyl, 3-hydroxy-3-phenylpropanoyl, 3-hydroxy-3-butanoyl, N,N-dimethoxyethylaminocarbonyl, N-allyl-N-methylaminocarbonyl, 1-piperidinocarbonyl, 4-oxo-piperidi-1-nocarbonyl, 4-(1,3-dioxan)piperidnocarbonyl, piperidin-1-ylmethyl, benzoyl, 1-hydroxybenzyl, 1-hydroxyiminoethyl, 1-methoxyiminoethyl, 1-allyloxyiminoethyl, phenoxyiminoethyl, 1-ethoxyiminoethyl, 1-carboxymethoxyiminoethyl, 1-t-butyloxyiminoethyl, 1-benzyloxyiminoethyl, 1-(4-nitrobenzyl)oxyiminoethyl, 1-hydroxyiminomethyl, 1-hydroxyprop-2-ynyl, and but-2-enoyl.

In certain embodiments, R₁₁ is selected from hydroxymethyl, acetyl, 1-hydroxy-1-methylethyl, 1-hydroxyethyl, 1-hydroxyiminoethyl, 1-methoxyiminoethyl, 1-allyloxyiminoethyl, 1-phenoxyiminoethyl, 1-ethoxyiminoethyl, 1-tertbutoxyiminoethyl, 1-benzyloxyiminoethyl, hydroxyiminomethyl, methoxymethyl, methoxymethoxymethyl, 1-hydroxy-2,2,2-trifluoroethyl, cyclohexylcarbonyl, prop-2-ynylaminomethyl, 2,2,2-trifluoroethylaminomethyl, 2-hydroxymethoxymethyl, 2-cyanovinyl, 1-methoxy-2,2,2-trifluoroethyl, 4-oxopiperidinocarbonyl, 2,2,2-trifluoroethylcarbamoyl, pyrrolidinocarbonyl and piperidinocarbonyl.

In certain embodiments, R₁₂ is selected from hydrogen, F, Cl, Br, CN, an optionally substituted C₁-C₄ alkyl, an optionally substituted C₁-C₄ haloalkyl, an optionally substituted C₁-C₄ heteroalkyl, —OR₁₆, a phenyl that is optionally substituted with hydrogen, a halogen, an optionally substituted C₁-C₄ alkyl, an optionally substituted C₁-C₄ haloalkyl, and an optionally substituted C₁-C₄ heteroalkyl. In certain embodiments, R₁₂ is selected from F, Cl, Br, CN, an optionally substituted C₁-C₄ alkyl, an optionally substituted C₁-C₄ haloalkyl, an optionally substituted C₁-C₄ heteroalkyl, —OR₁₆, a phenyl that is optionally substituted with hydrogen, a halogen, an optionally substituted C₁-C₄ alkyl, an optionally substituted C₁-C₄ haloalkyl, and an optionally substituted C₁-C₄ heteroalkyl. In certain embodiments, R₁₂ is selected from hydrogen, F, Cl, Br, CN, and an optionally substituted C₁-C₄ alkyl. In certain embodiments, R₁₂ is selected from hydrogen, and an optionally substituted C₁-C₄ alkyl. In certain embodiments, R₁₂ is hydrogen. In certain embodiments, R₁₂ is an optionally substituted C₁-C₄ alkyl.

In certain embodiments, R₁₃ is selected from hydrogen, F, Cl, Br, CN, CONR₁₄R₁₅, an optionally substituted C₁-C₄ alkyl, an optionally substituted C₁-C₄ haloalkyl, and an optionally substituted C₁-C₄ heteroalkyl. In certain embodiments, R₁₃ is selected from F, Cl, Br, CN, CONR₁₄R₁₅, an optionally substituted C₁-C₄ alkyl, an optionally substituted C₁-C₄ haloalkyl, and an optionally substituted C₁-C₄ heteroalkyl. In certain embodiments, R₁₃ is selected from hydrogen, C₁-C₄ alkyl and CONR₁₄R₁₅. In certain embodiments, R₁₃ is selected from hydrogen, methyl, N,N-diethylaminocarbonyl, 1-pyrrolidinocarbonyl, 2-methylpyrrolidi-1-nocarbonyl, and 1-morpholinocarbonyl. In certain embodiments, R₁₃ is selected from methyl, N,N-diethylaminocarbonyl, 1-pyrrolidinocarbonyl, 2-methylpyrrolidi-1-nocarbonyl, and 1-morpholinocarbonyl.

In certain embodiments, R₁₁ and R₁₂ together form an optionally substituted 5-6 member ring and R₁₃ is selected from hydrogen, F, Cl, Br, CN, CONR₁₄R₁₅, an optionally substituted C₁-C₄ alkyl, an optionally substituted C₁-C₄ haloalkyl, and an optionally substituted C₁-C₄ heteroalkyl. In certain embodiments, R₁₂ and R₁₃ together form an optionally substituted 4-6 member ring and R₁₁, is selected from hydrogen, F, Cl, Br, CN, an optionally substituted C₁-C₄ alkyl, an optionally substituted C₁-C₄ haloalkyl, an optionally substituted C₁-C₄ heteroalkyl, —CONR₁₄R₁₅, and an optionally substituted aryl.

In certain embodiments, R₁₄ and R₁₅ are each independently selected from hydrogen, an optionally substituted C₁-C₄ alkyl, an optionally substituted C₁-C₄ haloalkyl, and an optionally substituted C₁-C₄ heteroalkyl.

In certain embodiments, R₁₄ and R₁₅ together form an optionally substituted 4-7 member ring.

In certain embodiments, R₁₆ is selected from hydrogen, an optionally substituted C₁-C₄ alkyl, an optionally substituted C₁-C₄ haloalkyl, an optionally substituted C₁-C₄ heteroalkyl, and an optionally substituted aryl. In certain embodiments in which R₁₆ is an optionally substituted aryl, R₁₆ is an optionally substituted phenyl. In certain of such embodiments, R₁₆ is an optionally substituted phenyl that is optionally substituted with a substituent selected from hydrogen, a halogen, an optionally substituted C₁-C₄ alkyl, an optionally substituted C₁-C₄ haloalkyl, and an optionally substituted C₁-C₄ heteroalkyl.

In certain embodiments, X is selected from O, S, and NR₁₇. In certain embodiments, X is O. In certain embodiments, X is S. In certain embodiments, X is NR₁₇. In certain embodiments, X is NR₁₇, and R₁₇ is hydrogen.

In certain embodiments, R₁₇ is selected from hydrogen and an optionally substituted C₁-C₄ alkyl.

In certain embodiments, R₁ is

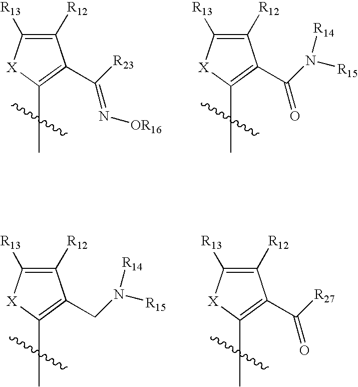

wherein the variables are as described elsewhere herein. In certain embodiments, R₂₃ is selected from among hydrogen, optionally substituted C₁-C₄ alkyl, optionally substituted C₁-C₄ alkenyl, optionally substituted C₁-C₄ alkynyl and optionally substituted aryl;

R₂₇ is selected from among hydrogen, optionally substituted C₁-C₄ alkyl, optionally substituted C₁-C₄ alkenyl, optionally substituted C₁-C₄ alkynyl, optionally substituted aryl, optionally substituted heteroaryl, optionally substituted cycloalkyl, and optionally substituted heterocyclyl; and the other variables are as described elsewhere herein.

In certain embodiments, R₁ is

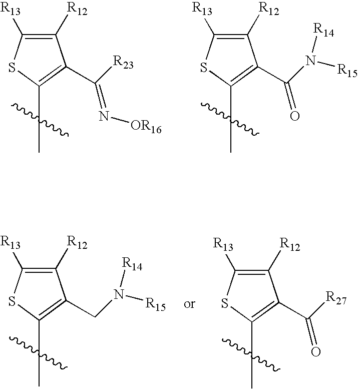

wherein the variables are as described elsewhere herein.

In certain embodiments, R₁ is

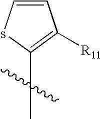

wherein the variables are as described elsewhere herein. In certain embodiments, R₁₁ is selected from hydroxyC₁-C₄alkyl, hydroxyiminoC₁-C₄alkyl, C₁-C₄alkoxyiminoC₁-C₄alkyl, C₁-C₄alkylcarbonyl, C₁-C₄alkenyloxyiminoC₁-C₄alkyl, aryloxyiminoC₁-C₄alkyl, aralkoxyiminoC₁-C₄alkyl, C₁-C₄alkoxyC₁-C₄alkyl, C₁-C₄alkoxyalkoxyC₁-C₄alkyl, hydroxyhaloC₁-C₄alkyl, cycloalkylcarbonyl, C₂-C₄alkynylaminoC₁-C₄alkyl, haloC₁-C₄alkylaminoC₁-C₄alkyl, hydroxyalkoxyC₁-C₄alkyl, cyanoC₂-C₄alkenyl, alkoxyhaloC₁-C₄alkyl, heterocyclylcarbonyl and haloalkylcarbamoyl.

In certain embodiments, R₁ is

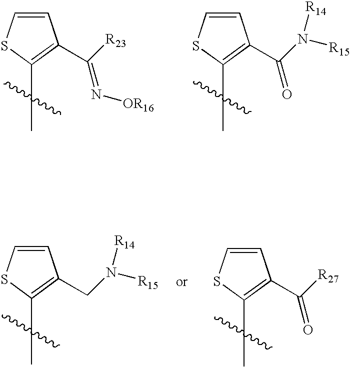

wherein the variables are as described elsewhere herein.

In certain embodiments, R₁ is

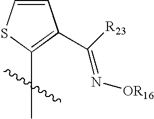

wherein the variables are as described elsewhere herein. In certain embodiments, R₁₆ is hydrogen, methyl, allyl, tert-butyl, and benzyl; and the other variables are as described elsewhere herein. In certain embodiments, R₂₃ is hydrogen or methyl.

In certain embodiments, R₁ is

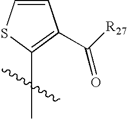

wherein the variables are as described elsewhere herein. In certain embodiments, R₂₇ is methyl, cyclohexyl, 4-oxo-piperidinyl, pyrrolidinyl, or piperidinyl and the other variables are as described elsewhere herein.

In certain embodiments, R₁ is

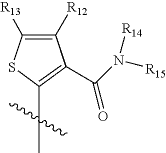

wherein the variables are as described elsewhere herein. In certain embodiments, R₁₄ and R₁₅ are each independently hydrogen, alkyl, haloalkyl or aryl, or R₁₄ and R₁₅ together with the nitrogen atom on which they are substituted form an optionally substituted heterocyclyl or optionally substituted heteroaryl ring. In certain embodiments, R₁₄ and R₁₅ are each independently hydrogen, methyl, trifluoroethyl, or R₁₄ and R₁₅ together with the nitrogen atom on which they are substituted form a pyrrolidinyl, 4-oxopiperidinyl or piperidinyl ring.

In certain embodiments, R₁ is

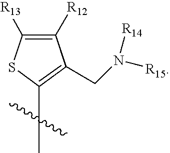

wherein the variables are as described elsewhere herein.

d. Exemplary Compounds

In certain embodiments, the compounds provided herein have Formula V

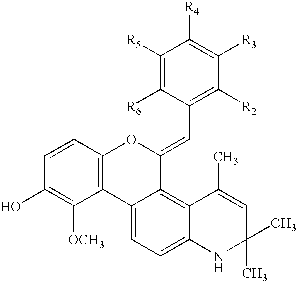

wherein the variables are as described elsewhere herein.

In certain embodiments, the compounds provided herein have Formula VI or VII

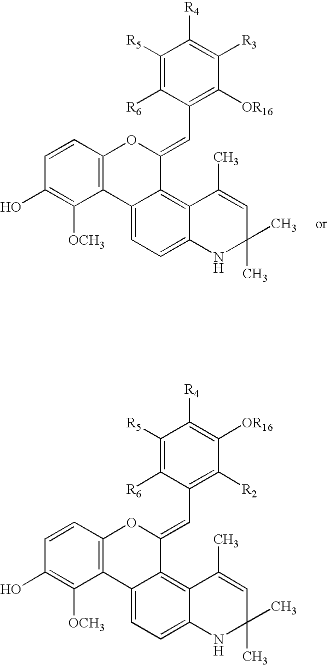

In certain embodiments, the compounds provided herein have Formula VIII

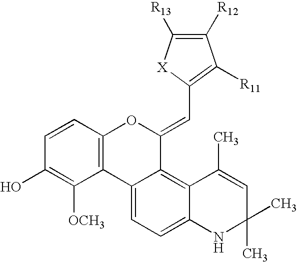

wherein the variables are as described elsewhere herein.

In certain embodiments, the compounds provided herein have Formula IX

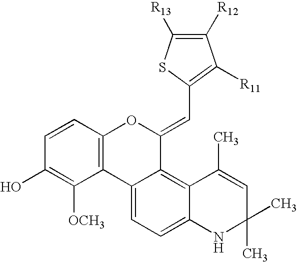

wherein the variables are as described elsewhere herein.

In certain embodiments, the compounds provided herein have Formula X

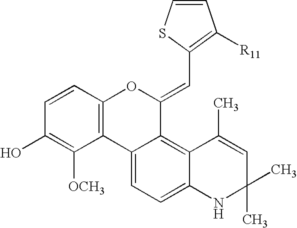

wherein the variables are as described elsewhere herein.

In certain embodiments, the compounds provided herein have Formula XI

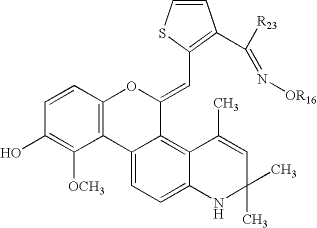

wherein the variables are as described elsewhere herein.

In certain embodiments, the compounds provided herein have Formula XII

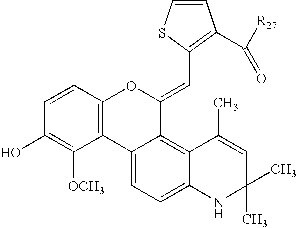

wherein the variables are as described elsewhere herein.

In certain embodiments, the compounds provided herein have Formula XIII

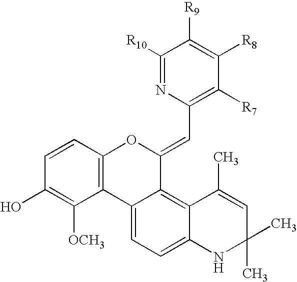

wherein the variables are as described elsewhere herein.

In certain embodiments, a compound of Formula I is a selective glucocorticoid receptor modulator. In certain embodiments, a compound of Formula I is a selective glucocorticoid receptor agonist. In certain embodiments, a compound of Formula I is a selective glucocorticoid receptor antagonist. In certain embodiments, a compound of Formula I is a selective glucocorticoid receptor partial agonist. In certain embodiments, a compound of Formula I is a tissue-specific selective glucocorticoid modulator. In certain embodiments, a compound of Formula I is a gene-specific selective glucocorticoid modulator. In certain embodiments, a compound of Formula I is a selective glucocorticoid receptor binding compound.

Any combination of the following Markush group and those described above for the various variables is contemplated herein.

TABLE A
Table of Markush (possible substituent groups in alternative)
Groups by Variable

| | Markush Group A | Markush Group B | Markush Group C | Markush Group D |
|---|---|---|---|---|
| 2 | hydrogen, F, Cl, Br, CN, | hydrogen, F, | hydrogen, | H |
| | an optionally substituted | Cl, optionally | —CONR₁₄R₁₅ | |
| | C₁-C₄ alkyl, an | substituted C₁- | | |
| | optionally substituted C₁- | C₄ alkyl, | | |
| | C₄ haloalkyl, an | —CONR₁₄R₁₅ | | |
| | optionally substituted C₁- | | | |
| | C₄ heteroalkyl, | | | |
| | —CONR₁₄R₁₅, —OR₁₆, | | | |
| | —SR₁₆, —SO₂NR₁₄R₁₅, and | | | |
| | an optionally substituted | | | |
| | aryl | | | |
| | R₂ and R₃ together form | | | |
| | an optionally substituted | | | |
| | 5-6 member ring | | | |
| 3 | hydrogen, F, Cl, Br, CN, | hydrogen, F, | optionally | H |
| | an optionally substituted | Cl, optionally | substituted | |
| | C₁-C₄ alkyl, an | substituted C₁- | C₁-C₂ alkyl, | |
| | optionally substituted C₁- | C₄ alkyl, | optionally | |
| | C₄ haloalkyl, an | optionally | substituted | |
| | optionally substituted C₁- | substituted C₁- | C₁-C₂ | |
| | C₄ heteroalkyl, —OR₁₆, | C₄ haloalkyl, | haloalkyl, | |
| | —SR₁₆ and an optionally | —OR₁₆ | —OR₁₆ | |
| | substituted aryl | | | |
| | R₂ and R₃ together form | | | |
| | an optionally substituted | | | |
| | 5-6 member ring | | | |
| | R₃ and R₄ together form | | | |
| | an optionally substituted | | | |
| | 4-6 member ring | | | |
| 4 | hydrogen, F, Cl, Br, CN, | hydrogen, F, | hydrogen, F, | H |
| | —OR₁₆, a ring, an | Cl, —OR₁₆, | optionally | |
| | optionally substituted C₁- | optionally | substituted | |
| | C₄ alkyl, an optionally | substituted C₁- | C₁-C₂ alkyl | |
| | substituted C₁-C₄ | C₄ alkyl | | |
| | haloalkyl, and an | | | |
| | optionally substituted C₁- | | | |
| | C₄ heteroalkyl | | | |
| | R₃ and R₄ together form | | | |
| | an optionally substituted | | | |
| | 4-6 member ring | | | |
| 5 | hydrogen, F, Cl, Br, | hydrogen, F, | CH₃ | H |
| | optionally substituted C₁- | Cl, Br, | | |
| | C₄ alkyl, and OCH₃ | optionally | | |
| | | substituted C₁- | | |
| | | C₂ alkyl | | |
| 6 | hydrogen and F | F | | H |
| 7 | hydrogen, F, Cl, Br, CN, | H, F, optionally | CH₃ | H |
| | an optionally substituted | substituted C₁- | | |
| | C₁-C₄ alkyl, an | C₄ alkyl | | |
| | optionally substituted C₁- | | | |
| | C₄ haloalkyl, an | | | |
| | optionally substituted C₁- | | | |
| | C₄ heteroalkyl, | | | |
| | —CONR₁₄R₁₅, and an | | | |
| | optionally substituted | | | |
| | aryl | | | |
| | R₇ and R₈ together form | | | |
| | an optionally substituted | | | |
| | 5-6 member ring | | | |
| 8 | hydrogen, F, Cl, Br, CN, | H, F, optionally | CH₃ | H |
| | an optionally substituted | substituted C₁- | | |
| | C₁-C₄ alkyl, an | C₄ alkyl | | |
| | optionally substituted C₁- | | | |
| | C₄ haloalkyl, an | | | |
| | optionally substituted C₁- | | | |
| | C₄ heteroalkyl, —OR₁₆, a | | | |
| | phenyl that is optionally | | | |
| | substituted with | | | |
| | hydrogen, a halogen, an | | | |
| | optionally substituted C₁- | | | |
| | C₄ alkyl, an optionally | | | |
| | substituted C₁-C₄ | | | |
| | haloalkyl, and an | | | |
| | optionally substituted C₁- | | | |
| | C₄ heteroalkyl | | | |
| | R₇ and R₈ together form | | | |
| | an optionally substituted | | | |
| | 5-6 member ring | | | |
| | R₈ and R₉ together form | | | |
| | an optionally substituted | | | |
| | 4-6 member ring | | | |
| 9 | hydrogen, F, Cl, Br, CN, | H, F, optionally | CH₃ | H |
| | an optionally substituted | substituted C₁- | | |
| | C1-C4 alkyl, an | C₄ alkyl | | |
| | optionally substituted | | | |
| | C1-C4 haloalkyl, and an | | | |
| | optionally substituted | | | |
| | C1-C4 heteroalkyl | | | |
| | R₈ and R₉ together form | | | |
| | an optionally substituted | | | |
| | 4-6 member ring | | | |
| 10 | hydrogen, F, Cl, CH₃, | H, F, CH₃ | CH₃ | H |
| | and OCH₃ | | | |
| 11 | hydrogen, F, Cl, Br, CN, | hydrogen, F, | —CONR₁₄R₁₅ | H |
| | an optionally substituted | Cl, | | |
| | C₁-C₄ alkyl, an | —CONR₁₄R₁₅ | | |
| | optionally substituted C₁- | | | |
| | C₄ haloalkyl, an | | | |
| | optionally substituted C₁- | | | |
| | C₄ heteroalkyl, | | | |
| | —CONR₁₄R₁₅, and an | | | |
| | optionally substituted | | | |
| | aryl | | | |
| | R₁₁ and R₁₂ together | | | |
| | form an optionally | | | |
| | substituted 5-6 member | | | |
| | ring | | | |
| 12 | hydrogen, F, Cl, Br, CN, | H, F, Cl, | CH₃ | H |
| | an optionally substituted | optionally | | |
| | C₁-C₄ alkyl, an | substituted C₁- | | |
| | optionally substituted C₁- | C₄ alkyl | | |
| | C₄ haloalkyl, an | | | |
| | optionally substituted C₁- | | | |
| | C₄ heteroalkyl, —OR₁₆, a | | | |
| | phenyl that is optionally | | | |
| | substituted with | | | |
| | hydrogen, a halogen, an | | | |
| | optionally substituted C₁- | | | |
| | C₄ alkyl, an optionally | | | |
| | substituted C₁-C₄ | | | |
| | haloalkyl, and an | | | |
| | optionally substituted C₁- | | | |
| | C₄ heteroalkyl | | | |
| | R₁₁ and R₁₂ together | | | |
| | form an optionally | | | |
| | substituted 5-6 member | | | |
| | ring | | | |
| | R₁₂ and R₁₃ together | | | |
| | form an optionally | | | |
| | substituted 4-6 member | | | |
| | ring | | | |
| 13 | R₁₃ is selected from | hydrogen, F, | —CONR₁₄R₁₅ | H |
| | hydrogen, F, Cl, Br, CN, | Cl, | | |
| | CONR₁₄R₁₅, an | —CONR₁₄R₁₅ | | |
| | optionally substituted C₁- | | | |
| | C₄ alkyl, an optionally | | | |
| | substituted C₁-C₄ | | | |
| | haloalkyl, and an | | | |
| | optionally substituted C₁- | | | |
| | C₄ heteroalkyl | | | |
| | R₁₂ and R₁₃ together | | | |
| | form an optionally | | | |
| | substituted 4-6 member | | | |
| | ring | | | |
| 14 | hydrogen, an optionally | H, optionally | CH₃ | H |
| | substituted C₁-C₄ alkyl, | substituted C₁- | | |
| | an optionally substituted | C₄ alkyl | | |
| | C₁-C₄ haloalkyl, and an | | | |
| | optionally substituted C₁- | | | |
| | C₄ heteroalkyl | | | |
| | R₁₄ and R₁₅ together | | | |
| | form an optionally | | | |
| | substituted 4-7 member | | | |
| | ring | | | |
| 15 | hydrogen, an optionally | H, optionally | CH₃ | H |
| | substituted C₁-C₄ alkyl, | substituted C₁- | | |
| | an optionally substituted | C₄ alkyl | | |
| | C₁-C₄ haloalkyl, and an | | | |
| | optionally substituted C₁- | | | |
| | C₄ heteroalkyl | | | |
| | R₁₄ and R₁₅ together | | | |
| | form an optionally | | | |
| | substituted 4-7 member | | | |
| | ring | | | |
| 16 | R₁₆ is selected from | H, optionally | CH₃ | H |
| | hydrogen, an optionally | substituted C₁- | | |
| | substituted C₁-C₄ alkyl, | C₄ alkyl | | |
| | an optionally substituted | | | |
| | C₁-C₄ haloalkyl, an | | | |
| | optionally substituted C₁- | | | |
| | C₄ heteroalkyl, and an | | | |
| | optionally substituted | | | |
| | aryl | | | |
| 17 | hydrogen and an | H, optionally | CH₃ | H |
| | optionally substituted C₁- | substituted C₁- | | |
| | C₄ alkyl | C₂ alkyl | | |
| | O, S, and NR₁₇ | O, S | S | O |

In certain embodiments, the compound provided herein is selected from
(Z)-5-(3′-trifluoromethylbenzylidene)-1,2-dihydro-9-hydroxy-10-methoxy-2,2,4-trimethyl-5H-chromeno[3,4-f]quinoline (compound 11);
(Z)-5-(2′-fluorobenzylidene)-1,2-dihydro-9-hydroxy-10-methoxy-2,2,4-trimethyl-5H-chromeno[3,4-f]quinoline (compound 12);
(Z)-5-(3′-chlorobenzylidene)-1,2-dihydro-9-hydroxy-10-methoxy-2,2,4-trimethyl-5H-chromeno[3,4-f]quinoline (compound 13);
(Z)-5-(2′,5′-dichlorobenzylidene)-1,2-dihydro-9-hydroxy-10-methoxy-2,2,4-trimethyl-5H-chromeno[3,4-f]quinoline (compound 14);
(Z)-5-(3′-methoxybenzylidene)-1,2-dihydro-9-hydroxy-10-methoxy-2,2,4-trimethyl-5H-chromeno[3,4-f]quinoline (compound 15);
(Z)-5-(2′-chlorobenzylidene)-1,2-dihydro-9-hydroxy-10-methoxy-2,2,4-trimethyl-5H-chromeno[3,4-f]quinoline (compound 16);
(Z)-5-(4′-chlorobenzylidene)-1,2-dihydro-9-hydroxy-10-methoxy-2,2,4-trimethyl-5H-chromeno[3,4-f]quinoline (compound 17);
(Z)-5-(3′-methylbenzylidene)-1,2-dihydro-9-hydroxy-10-methoxy-2,2,4-trimethyl-5H-chromeno[3,4-f]quinoline (compound 18);
(Z)-5-(4′-methylbenzylidene)-1,2-dihydro-9-hydroxy-10-methoxy-2,2,4-trimethyl-5H-chromeno[3,4-f]quinoline (compound 19);
(Z)-5-(4′-methoxybenzylidene)-1,2-dihydro-9-hydroxy-10-methoxy-2,2,4-trimethyl-5H-chromeno[3,4-f]quinoline (compound 20);
(Z)-5-(2′-bromobenzylidene)-1,2-dihydro-9-hydroxy-10-methoxy-2,2,4-trimethyl-5H-chromeno[3,4-f]quinoline (compound 21);
(Z)-5-(3′-trifluoromethoxybenzylidene)-1,2-dihydro-9-hydroxy-10-methoxy-2,2,4-trimethyl-5H-chromeno[3,4-f]quinoline (compound 22);
(Z)-5-(3′,5′-dichlorobenzylidene)-1,2-dihydro-9-hydroxy-10-methoxy-2,2,4-trimethyl-5H-chromeno[3,4-f]quinoline (compound 23);
(Z)-5-(3′-bromobenzylidene)-1,2-dihydro-9-hydroxy-10-methoxy-2,2,4-trimethyl-5H-chromeno[3,4-f]quinoline (compound 24);
(Z)-5-(2′-chloro-4′-fluorobenzylidene)-1,2-dihydro-9-hydroxy-10-methoxy-2,2,4-trimethyl-5H-chromeno[3,4-f]quinoline (compound 25);
(Z)-5-(4′-trifluoromethoxybenzylidene)-1,2-dihydro-9-hydroxy-10-methoxy-2,2,4-trimethyl-5H-chromeno[3,4-f]quinoline (compound 26);
(Z)-5-(3′-trifluoromethylthiobenzylidene)-1,2-dihydro-9-hydroxy-10-methoxy-2,2,4-trimethyl-5H-chromeno[3,4-f]quinoline (compound 27);
(Z)-5-(2′-fluoro-3′-methylbenzylidene)-1,2-dihydro-9-hydroxy-10-methoxy-2,2,4-trimethyl-5H-chromeno[3,4-f]quinoline (compound 28);
(Z)-5-(2′-fluoro-3′-trifluoromethylbenzylidene)-1,2-dihydro-9-hydroxy-10-methoxy-2,2,4-trimethyl-5H-chromeno[3,4-f]quinoline (compound 29);
(Z)-5-(3′,4′-dichlorobenzylidene)-1,2-dihydro-9-hydroxy-10-methoxy-2,2,4-trimethyl-5H-chromeno[3,4-f]quinoline (compound 30);
(Z)-5-(4′-chloro-3′-trifluoromethylbenzylidene)-1,2-dihydro-9-hydroxy-10-methoxy-2,2,4-trimethyl-5H-chromeno[3,4-f]quinoline (compound 31);
(Z)-5-(3′,5′-di(trifluoromethy)lbenzylidene)-1,2-dihydro-9-hydroxy-10-methoxy-2,2,4-trimethyl-5H-chromeno[3,4-f]quinoline (compound 32);
(Z)-5-(3′-fluoro-5′-trifluoromethylbenzylidene)-1,2-dihydro-9-hydroxy-10-methoxy-2,2,4-trimethyl-5H-chromeno[3,4-f]quinoline (compound 33);
(Z)-5-(2′,4′,5′-trifluorobenzylidene)-1,2-dihydro-9-hydroxy-10-methoxy-2,2,4-trimethyl-5H-chromeno[3,4-f]quinoline (compound 34);
(Z)-5-(2′-methylbenzylidene)-1,2-dihydro-9-hydroxy-10-methoxy-2,2,4-trimethyl-5H-chromeno[3,4-f]quinoline (compound 35);
(Z)-5-(4′-ethylbenzylidene)-1,2-dihydro-9-hydroxy-10-methoxy-2,2,4-trimethyl-5H-chromeno[3,4-f]quinoline (compound 38);
(Z)-5-(5′-fluoro-2′-methylbenzylidene)-1,2-dihydro-9-hydroxy-10-methoxy-2,2,4-trimethyl-5H-chromeno[3,4-f]quinoline (compound 37);
(Z)-5-(2′-chloro-6′-fluorobenzylidene)1,2-dihydro-9-hydroxy-10-methoxy-2,2,4-trimethyl-5H-chromeno[3,4-f]quinoline (compound 36);
(Z)-5-(4′-isopropylbenzylidene)-1,2-dihydro-9-hydroxy-10-methoxy-2,2,4-trimethyl-5H-chromeno[3,4-f]quinoline (compound 39);
(Z)-5-(4′-bromobenzylidene)-1,2-dihydro-9-hydroxy-10-methoxy-2,2,4-trimethyl-5H-chromeno[3,4-f]quinoline (compound 40);
(Z)-5-(3′-fluoro-4′-methylbenzylidene)-1,2-dihydro-9-hydroxy-10-methoxy-2,2,4-trimethyl-5H-chromeno[3,4-f]quinoline (compound 41);
(Z)-5-(2′-(6′-methyl-pyridinylmethylidiene))-1,2-dihydro-9-hydroxy-10-methoxy-2,2,4-trimethyl-5H-chromeno[3,4-f]quinoline (compound 42);
(Z)-5-(2′-methyl-3′-trifluoromethylbenzylidene)-1,2-dihydro-9-hydroxy-10-methoxy-2,2,4-trimethyl-5H-chromeno[3,4-f]quinoline (compound 43);
(Z)-5-(4′-benzyloxybenzylidene)-1,2-dihydro-9-hydroxy-10-methoxy-2,2,4-trimethyl-5H-chromeno[3,4-f]quinoline (compound 44);
(Z)-5-(2′-phenylbenzylidene)-1,2-dihydro-9-hydroxy-10-methoxy-2,2,4-trimethyl-5H-chromeno[3,4-f]quinoline (compound 46);
(Z)-5-(4′-phenylbenzylidene)-1,2-dihydro-9-hydroxy-10-methoxy-2,2,4-trimethyl-5H-chromeno[3,4-f]quinoline (compound 47);
(Z)-5-(3′-methyl-4′-fluorobenzylidene)-1,2-dihydro-9-hydroxy-10-methoxy-2,2,4-trimethyl-5H-chromeno[3,4-f]quinoline (compound 48);
(Z)-5-(4′-cyclohexylbenzylidene)-1,2-dihydro-9-hydroxy-10-methoxy-2,2,4-trimethyl-5H-chromeno[3,4-f]quinoline (compound 49);
(Z)-5-(2′-chloro-3′-trifluoromethylbenzylidene)-1,2-dihydro-9-hydroxy-10-methoxy-2,2,4-trimethyl-5H-chromeno[3,4-f]quinoline (compound 51);
(Z)-5-(3′-phenylbenzylidene)-1,2-dihydro-9-hydroxy-10-methoxy-2,2,4-trimethyl-5H-chromeno[3,4-f]quinoline (compound 52);
(Z)-5-(3′-chloro-4′-trifluoromethoxybenzylidene)-1,2-dihydro-9-hydroxy-10-methoxy-2,2,4-trimethyl-5H-chromeno[3,4-f]quinoline (compound 54);
(Z)-5-(2′,6′-difluoro-3′-chlorobenzylidene)-1,2-dihydro-9-hydroxy-10-methoxy-2,2,4-trimethyl-5H-chromeno[3,4-f]quinoline (compound 55);
(Z)-5-(2′-chloro-3′,6′-difluorobenzylidene)-1,2-dihydro-9-hydroxy-10-methoxy-2,2,4-trimethyl-5H-chromeno[3,4-f]quinoline (compound 56);
(Z)-5-(4′-methyl-3′-trifluoromethylbenzylidene)-1,2-dihydro-9-hydroxy-10-methoxy-2,2,4-trimethyl-5H-chromeno[3,4-f]quinoline (compound 58);
(Z)-5-(2′-fluoro-4′-chlorobenzylidene)-1,2-dihydro-9-hydroxy-10-methoxy-2,2,4-trimethyl-5H-chromeno[3,4-f]quinoline (compound 59);
(Z)-5-(2′,3′-difluoro-4′-methylbenzylidene)-1,2-dihydro-9-hydroxy-10-methoxy-2,2,4-trimethyl-5H-chromeno[3,4-f]quinoline (compound 60);
(Z)-5-(2′,3′,5′,6′-tetrafluoro-4′-trifluoromethylbenzylidene)-1,2-dihydro-9-hydroxy-10-methoxy-2,2,4-trimethyl-5H-chromeno[3,4-f]quinoline (compound 61);
(Z)-5-(2′-(3′-(dimethylaminocarbonyl)furanylmethyllidene))-1,2-dihydro-9-hydroxy-10-methoxy-2,2,4-trimethyl-5H-chromeno[3,4-f]quinoline (compound 62);
(Z)-5-(4′-vinylbenzylidene)-1,2-dihydro-9-hydroxy-10-methoxy-2,2,4-trimethyl-5H-chromeno[3,4-f]quinoline (compound 63);
(Z)-5-(2′-Chloro-6′-fluoro-5′-methylbenzylidene)-1,2-dihydro-9-hydroxy-10-methoxy-2,2,4-trimethyl-5H-chromeno[3,4-f]quinoline (compound 64);
(Z)-5-(2′-trifluoromethoxybenzylidene)-1,2-dihydro-9-hydroxy-10-methoxy-2,2,4-trimethyl-5H-chromeno[3,4-f]quinoline (compound 65);
(Z)-5-(2′-trifluoromethylthiobenzylidene)-1,2-dihydro-9-hydroxy-10-methoxy-2,2,4-trimethyl-5H-chromeno[3,4-f]quinoline (compound 66);
(Z)-5-(3′,4′-methylenedioxybenzylidene)-1,2-dihydro-9-hydroxy-10-methoxy-2,2,4-trimethyl-5H-chromeno[3,4-f]quinoline (compound 67);
(Z)-5-(3′-chloro-2′-fluorobenzylidene)-1,2-dihydro-9-hydroxy-10-methoxy-2,2,4-trimethyl-5H-chromeno[3,4-f]quinoline (compound 68);
(Z)-5-(4′-(4″-methylbenzyloxy)benzylidene)-1,2-dihydro-9-hydroxy-10-methoxy-2,2,4-trimethyl-5H-chromeno[3,4-f]quinoline (compound 70);
(Z)-5-(3′,5′-di-tert-butylbenzylidene)-1,2-dihydro-9-hydroxy-10-methoxy-2,2,4-trimethyl-5H-chromeno[3,4-f]quinoline (compound 71);
(Z)-5-(3′-(2″,2″-difluoroethoxy)benzylidene)-1,2-dihydro-9-hydroxy-10-methoxy-2,2,4-trimethyl-5H-chromeno[3,4-f]quinoline (compound 72);
(Z)-5-(2′,5′-dimethylbenzylidene)-1,2-dihydro-9-hydroxy-10-methoxy-2,2,4-trimethyl-5H-chromeno[3,4-f]quinoline (compound 73);
(Z)-5-(3′-(3″-thienyl)benzylidene)-1,2-dihydro-9-hydroxy-10-methoxy-2,2,4-trimethyl-5H-chromeno[3,4-f]quinoline (compound 74);
(Z)-5-(2′-diethylaminocarbonylbenzylidene)-1,2-dihydro-9-hydroxy-10-methoxy-2,2,4-trimethyl-5H-chromeno[3,4-f]quinoline (compound 75);
(Z)-5-(3′-(4″,4″,4″-trifluorobutoxy)benzylidene)-1,2-dihydro-9-hydroxy-10-methoxy-2,2,4-trimethyl-5H-chromeno[3,4-f]quinoline (compound 76);
(Z)-5-(3′-(2″,4″-difluorophenyl)benzylidene)-1,2-dihydro-9-hydroxy-10-methoxy-2,2,4-trimethyl-5H-chromeno[3,4-f]quinoline (compound 77);
(Z)-5-(3′-(3″-pyridyl)benzylidene)-1,2-dihydro-9-hydroxy-10-methoxy-2,2,4-trimethyl-5H-chromeno[3,4-f]quinoline (compound 78);
(Z)-5-(2′-(3″-benzenecarbaldehyde)benzylidene)-1,2-dihydro-9-hydroxy-10-methoxy-2,2,4-trimethyl-5H-chromeno[3,4-f]quinoline (compound 79);
(Z)-5-(3′,5′-dimethylbenzylidene)-1,2-dihydro-9-hydroxy-10-methoxy-2,2,4-trimethyl-5H-chromeno[3,4-f]quinoline (compound 80);
(Z)-5-(3′,4′-dimethylbenzylidene)-1,2-dihydro-9-hydroxy-10-methoxy-2,2,4-trimethyl-5H-chromeno[3,4-f]quinoline (compound 81);
(Z)-5-(2′-(diethylamino)carbonyl-6′-fluorobenzylidene)-1,2-dihydro-9-hydroxy-10-methoxy-2,2,4-trimethyl-5H-chromeno[3,4-f]quinoline (compound 82);
(Z)-5-(2′-(diethylamino)carbonyl-4′-fluorobenzylidene)-1,2-dihydro-9-hydroxy-10-methoxy-2,2,4-trimethyl-5H-chromeno[3,4-f]quinoline (compound 83);
(Z)-5-(2′-(methylbenzylamino)carbonyl-6′-fluorobenzylidene)-1,2-dihydro-9-hydroxy-10-methoxy-2,2,4-trimethyl-5H-chromeno[3,4-f]quinoline (compound 84);
(Z)-5-(2′-(dimethylamino)carbonyl-5′-bromo-fluorobenzylidene)-1,2-dihydro-9-hydroxy-10-methoxy-2,2,4-trimethyl-5H-chromeno[3,4-f]quinoline (compound 85);
(Z)-5-(3′-(2″-fluoroethoxy)benzylidene)-1,2-dihydro-9-hydroxy-10-methoxy-2,2,4-trimethyl-5H-chromeno[3,4-f]quinoline (compound 86);
(Z)-5-(3′-(2″,2″,3″,3″-tetrafluoropropoxy)benzylidene)-1,2-dihydro-9-hydroxy-10-methoxy-2,2,4-trimethyl-5H-chromeno[3,4-f]quinoline (compound 87);
(Z)-5-(3′-(4″-fluorobenzyloxy)benzylidene)-1,2-dihydro-9-hydroxy-10-methoxy-2,2,4-trimethyl-5H-chromeno[3,4-f]quinoline (compound 88);
(Z)-5-(3′-(2″-fluorobenzyloxy)benzylidene)-1,2-dihydro-9-hydroxy-10-methoxy-2,2,4-trimethyl-5H-chromeno[3,4-f]quinoline (compound 89);
(Z)-5-(2′-(pyrrolidinecarbonyl)benzylidene)-1,2-dihydro-9-hydroxy-10-methoxy-2,2,4-trimethyl-5H-chromeno[3,4-f]quinoline (compound 90);
(Z)-5-(2′-(pyrrolidinecarbonyl)-5′-bromobenzylidene)-1,2-dihydro-9-hydroxy-10-methoxy-2,2,4-trimethyl-5H-chromeno[3,4-f]quinoline (compound 91);
(Z)-5-(2′-(dimethylaminocarbonyl)-4′-fluorobenzylidene)-1,2-dihydro-9-hydroxy-10-methoxy-2,2,4-trimethyl-5H-chromeno[3,4-f]quinoline (compound 92);
(Z)-5-(2′-(pyrrolidinecarbonyl)-5′-methylbenzylidene)-1,2-dihydro-9-hydroxy-10-methoxy-2,2,4-trimethyl-5H-chromeno[3,4-f]quinoline (compound 93);
(Z)-5-(2′-(pyrrolidinecarbonyl)-4′-fluorobenzylidene)-1,2-dihydro-9-hydroxy-10-methoxy-2,2,4-trimethyl-5H-chromeno[3,4-f]quinoline (compound 94);
(Z)-5-(3′-(4″-fluorophenoxy)benzylidene)-1,2-dihydro-9-hydroxy-10-methoxy-2,2,4-trimethyl-5H-chromeno[3,4-f]quinoline (compound 95);
(Z)-5-(2′-(morpholinecarbonyl)-4′-fluorobenzylidene)-1,2-dihydro-9-hydroxy-10-methoxy-2,2,4-trimethyl-5H-chromeno[3,4-f]quinoline (compound 96);
(Z)-5-(8′-(6′-fluoro-benzo-1′,3′-dioxan-methylidiene))-1,2-dihydro-9-hydroxy-10-methoxy-2,2,4-trimethyl-5H-chromeno[3,4-f]quinoline (compound 97);
(Z)-5-(2′-dimethylaminocarbonyl-3′-methoxybenzylidene)-1,2-dihydro-9-hydroxy-10-methoxy-2,2,4-trimethyl-5H-chromeno[3,4-f]quinoline (compound 98);
(Z)-5-(2′-(4″-methylpiperazinecarbonyl)-4′-fluorobenzylidene)-1,2-dihydro-9-hydroxy-10-methoxy-2,2,4-trimethyl-5H-chromeno[3,4-f]quinoline (compound 99);
(Z)-5-(2′-methyl-3′-phenylbenzylidene)-1,2-dihydro-9-hydroxy-10-methoxy-2,2,4-trimethyl-5H-chromeno[3,4-f]quinoline (compound 100);
(Z)-5-(3′,5′-di-methoxybenzylidene)-1,2-dihydro-9-hydroxy-10-methoxy-2,2,4-trimethyl-5H-chromeno[3,4-f]quinoline (compound 101);
(Z)-5-(2′-(piperidinecarbonyl)-4′-fluorobenzylidene)-1,2-dihydro-9-hydroxy-10-methoxy-2,2,4-trimethyl-5H-chromeno[3,4-f]quinoline (compound 102);
(Z)-5-(2′-dimethylaminosulphonyl-4′-fluorobenzylidene)-1,2-dihydro-9-hydroxy-10-methoxy-2,2,4-trimethyl-5H-chromeno[3,4-f]quinoline (compound 103);
(Z)-5-(3′-phenoxybenzylidene)-1,2-dihydro-9-hydroxy-10-methoxy-2,2,4-trimethyl-5H-chromeno[3,4-f]quinoline (compound 104);
(Z)-5-(2′-(ethylmethylamino)carbonyl-4′-fluorobenzylidene)-1,2-dihydro-9-hydroxy-10-methoxy-2,2,4-trimethyl-5H-chromeno[3,4-f]quinoline (compound 105);
(Z)-5-(2′-(cyclohexylmethylamino)carbonyl-4′-fluorobenzylidene)-1,2-dihydro-9-hydroxy-10-methoxy-2,2,4-trimethyl-5H-chromeno[3,4-f]quinoline (compound 106);
(Z)-5-(2′-cyanobenzylidene)-1,2-dihydro-9-hydroxy-10-methoxy-2,2,4-trimethyl-5H-chromeno[3,4-f]quinoline (compound 107);
(Z)-5-(2′,3′,5′,6′-tetrafluoro-4′-methoxybenzylidene)-1,2-dihydro-9-hydroxy-10-methoxy-2,2,4-trimethyl-5H-chromeno[3,4-f]quinoline (compound 108);
(Z)-5-(3′-hydroxybenzylidene)-1,2-dihydro-9-hydroxy-10-methoxy-2,2,4-trimethyl-5H-chromeno[3,4-f]quinoline (compound 109);
(Z)-5-(2′-(piperidinesulphonyl)-4′-fluorobenzylidene)-1,2-dihydro-9-hydroxy-10-methoxy-2,2,4-trimethyl-5H-chromeno[3,4-f]quinoline (compound 110);
(Z)-5-(1′-napthylmethylidene)-1,2-dihydro-9-hydroxy-10-methoxy-2,2,4-trimethyl-5H-chromeno[3,4-f]quinoline (compound 111);
(Z)-5-(3′-methyl-4′-methoxybenzylidene)-1,2-dihydro-9-hydroxy-10-methoxy-2,2-cyclohexyl-4-methyl-5H-chromeno[3,4-f]quinoline (compound 112);
(Z)-5-(2′,5′-dimethoxybenzylidene)-1,2-dihydro-9-hydroxy-10-methoxy-2,2-cyclohexyl-4-methyl-5H-chromeno[3,4-f]quinoline (compound 113);
(Z)-5-(2′,3′-methylenedioxybenzylidene)-1,2-dihydro-9-hydroxy-10-methoxy-2,2,4-trimethyl-5H-chromeno[3,4-f]quinoline (compound 114);
(Z)-5-(2′,3′-ethylenedioxybenzylidene)-1,2-dihydro-9-hydroxy-10-methoxy-2,2,4-trimethyl-5H-chromeno[3,4-f]quinoline (compound 115);
(Z)-5-(4′-hydroxybenzylidene)-1,2-dihydro-9-hydroxy-10-methoxy-2,2,4-trimethyl-5H-chromeno[3,4-f]quinoline (compound 116);
(Z)-5-(2′-cyano-3′-methylbenzylidene)-1,2-dihydro-9-hydroxy-10-methoxy-2,2,4-trimethyl-5H-chromeno[3,4-f]quinoline (compound 117);
(Z)-5-(3′-chloro-2′-cyanobenzylidene)-1,2-dihydro-9-hydroxy-10-methoxy-2,2,4-trimethyl-5H-chromeno[3,4-f]quinoline (compound 118);
(Z)-5-(5′-bromo-2′-cyano-benzylidene)-1,2-dihydro-9-hydroxy-10-methoxy-2,2,4-trimethyl-5H-chromeno[3,4-f]quinoline (compound 119);
(Z)-5-(8′-(6′-chloro-benzo-1′,3′-dioxan-methylidiene)-1,2-dihydro-9-hydroxy-10-methoxy-2,2,4-trimethyl-5H-chromeno[3,4-f]quinoline (compound 120);
(Z)-5-(2′-chloro-3′,4′-dimethoxybenzylidene)-1,2-dihydro-9-hydroxy-10-methoxy-2,2,4-trimethyl-5H-chromeno[3,4-f]quinoline (compound 121);
(Z)-5-(2′-cyano-3′-fluorobenzylidene)-1,2-dihydro-9-hydroxy-10-methoxy-2,2,4-trimethyl-5H-chromeno[3,4-f]quinoline (compound 122);
(Z)-5-(8′-(6′-methyl-benzo-1′,3′-dioxan-methylidiene))-1,2-dihydro-9-hydroxy-10-methoxy-2,2,4-trimethyl-5H-chromeno[3,4-f]quinoline (compound 123);
(Z)-5-(2′-cyano-5′-methylbenzylidene)-1,2-dihydro-9-hydroxy-10-methoxy-2,2,4-trimethyl-5H-chromeno[3,4-f]quinoline (compound 124);
(Z)-5-(8′-(5′,6′-difluoro-benzo-1′,3′-dioxan-methylidiene))-1,2-dihydro-9-hydroxy-10-methoxy-2,2,4-trimethyl-5H-chromeno[3,4-f]quinoline (compound 125);
(Z)-5-(3′-(3″,5″-dichlorophenoxy)benzylidene)-1,2-dihydro-9-hydroxy-10-methoxy-2,2,4-trimethyl-5H-chromeno[3,4-f]quinoline (compound 126);
(Z)-5-(3′-(4″-methoxyphenoxy)benzylidene)-1,2-dihydro-9-hydroxy-10-methoxy-2,2,4-trimethyl-5H-chromeno[3,4-f]quinoline (compound 127);
(Z)-5-(3′-(3″,4″-dichlorophenoxy)benzylidene)-1,2-dihydro-9-hydroxy-10-methoxy-2,2,4-trimethyl-5H-chromeno[3,4-f]quinoline (compound 128);
(Z)-5-(3′-(4″-methylphenoxy)benzylidene)-1,2-dihydro-9-hydroxy-10-methoxy-2,2,4-trimethyl-5H-chromeno[3,4-f]quinoline (compound 129);
(Z)-5-(3′-(4″-chlorophenoxy)benzylidene)-1,2-dihydro-9-hydroxy-10-methoxy-2,2,4-trimethyl-5H-chromeno[3,4-f]quinoline (compound 130);
(Z)-5-(3′-(3″-trifluoromethoxyphenoxy)benzylidene)-1,2-dihydro-9-hydroxy-10-methoxy-2,2,4-trimethyl-5H-chromeno[3,4-f]quinoline (compound 131);
(Z)-5-(2′-(3′-(dimethylaminocarbonyl)thienylmethylidene))-1,2-dihydro-9-hydroxy-10-methoxy-2,2,4-trimethyl-5H-chromeno[3,4-f]quinoline (compound 132);
(Z)-5-(2′-(3′-(ethylmethylaminocarbonyl)thienylmethylidene))-1,2-dihydro-9-hydroxy-10-methoxy-2,2,4-trimethyl-5H-chromeno[3,4-f]quinoline (compound 134);
(Z)-5-(2′-(3′-(morpholinocarbonyl)thienylmethylidene)-1,2-dihydro-9-hydroxy-10-methoxy-2,2,4-trimethyl-5H-chromeno[3,4-f]quinoline (compound 135);
(Z)-5-(2′-(3′-(cyclohexylmethylaminocarbonyl)thienylmethylidene))-1,2-dihydro-9-hydroxy-10-methoxy-2,2,4-trimethyl-5H-chromeno[3,4-f]quinoline (compound 136);
(Z)-5-(2′-(3′-(pyrrolidinocarbonyl)thienylmethylidene))-1,2-dihydro-9-hydroxy-10-methoxy-2,2,4-trimethyl-5H-chromeno[3,4-f]quinoline (compound 137);
(Z)-5-(2′-(3′-(di(methoxyethyl)aminocarbonyl)thienylmethylidene)-1,2-dihydro-9-hydroxy-10-methoxy-2,2,4-trimethyl-5H-chromeno[3,4-f]quinoline (compound 138);
(Z)-5-(2′-(3′-(allylmethylaminocarbonyl)thienylmethylidene))-1,2-dihydro-9-hydroxy-10-methoxy-2,2,4-trimethyl-5H-chromeno[3,4-f]quinoline (compound 139);
(Z)-5-(2′-(3′-(piperidinocarbonyl)thienylmethylidene))-1,2-dihydro-9-hydroxy-10-methoxy-2,2,4-trimethyl-5H-chromeno[3,4-f]quinoline (compound 140);
(Z)-5-(2′-(3′-piperidinecarbonyl-4″-(1,3-dioxan)thienylmethylidene)-1,2-dihydro-9-hydroxy-10-methoxy-2,2,4-trimethyl-5H-chromeno[3,4-f]quinoline (compound 141);
(Z)-5-(2′-(5′-(diethylaminocarbonyl)thienylmethylidene))-1,2-dihydro-9-hydroxy-10-methoxy-2,2,4-trimethyl-5H-chromeno[3,4-f]quinoline (compound 142);
(Z)-5-(2′-(5′-(pyrrolidinocarbonyl)thienylmethylidene))-1,2-dihydro-9-hydroxy-10-methoxy-2,2,4-trimethyl-5H-chromeno[3,4-f]quinoline (compound 143);
(Z)-5-(2′-(5′-(2″-methylpyrrolidinocarbonyl)thienylmethylidene))-1,2-dihydro-9-hydroxy-10-methoxy-2,2,4-trimethyl-5H-chromeno[3,4-f]quinoline (compound 144);
(Z)-5-(2′-(5′-(morpholinocarbonyl)thienylmethylidene))-1,2-dihydro-9-hydroxy-10-methoxy-2,2,4-trimethyl-5H-chromeno[3,4-f]quinoline (compound 145);
(Z)-5-(2′-(3′-dimethylaminocarbonyl-5′-methylfuranylmethylidene))-1,2-dihydro-9-hydroxy-10-methoxy-2,2,4-trimethyl-5H-chromeno[3,4-f]quinoline (compound 146);
(Z)-5-(2′-(3′-cyclohexylmethylaminocarbonyl-5′-methylfuranylmethylidene))-1,2-dihydro-9-hydroxy-10-methoxy-2,2,4-trimethyl-5H-chromeno[3,4-f]quinoline (compound 147);
(Z)-5-(4′-(2″-fluorophenyl)benzylidene)1,2-dihydro-9-hydroxy-10-methoxy-2,2,4-trimethyl-5H-chromeno[3,4-f]quinoline (compound 148);
(Z)-5-(3′-(2″-Fluorophenyl)benzylidene)1,2-dihydro-9-hydroxy-10-methoxy-2,2,4-trimethyl-5H-chromeno(3,4-f)quinoline (compound 149);
(Z)-5-(2′-chloro-3′-methylbenzylidene)1,2-dihydro-9-hydroxy-10-methoxy-2,2,4-trimethyl-5H-chromeno[3,4-f]quinoline (compound 150);
(Z)-5-(2′-(5′-Methyl-3′-(piperidinocarbonyl)furanylmethylidene))1,2-dihydro-9-hydroxy-10-methoxy-2,2,4-trimethyl-5H-chromeno(3,4-f)quinoline (compound 151);
(Z)-5-(2′-(5′-Methyl-3′-(piperidinecarbonyl)thienylmethylidene))1,2-dihydro-9-hydroxy-10-methoxy-2,2,4-trimethyl-5H-chromeno(3,4-f)quinoline (compound 152);
(Z)-5-(2′-(3′-Diethylcarbamoyl-1′,5′-dimethyl-1′H-pyrrolylmethylidene))1,2-dihydro-9-hydroxy-10-methoxy-2,2,4-trimethyl-5H-chromeno(3,4-f)quinoline (compound 153);
(Z)-5-(3′-Methyl-2′-(pyrrolidinecarbonyl)benzylidene)1,2-dihydro-9-hydroxy-10-methoxy-2,2,4-trimethyl-5H-chromeno(3,4-f)quinoline (compound 154);
(Z)-5-(3′-Bromo-2′-(pyrrolidinecarbonyl)benzylidene)1,2-dihydro-9-hydroxy-10-methoxy-2,2,4-trimethyl-5H-chromeno(3,4-f)quinoline (compound 155);
(Z)-5-(3′-Chloro-2′-(pyrrolidinecarbonyl)benzylidene)1,2-dihydro-9-hydroxy-10-methoxy-2,2,4-trimethyl-5H-chromeno(3,4-f)quinoline (compound 156);
(Z)-5-(2′-(3′-Hydroxymethylthienylmethylidene))1,2-dihydro-9-hydroxy-10-methoxy-2,2,4-trimethyl-5H-chromeno(3,4-f)quinoline (compound 157);
(Z)-5-(2′-(Piperidinecarbonyl)benzylidene)1,2-dihydro-9-hydroxy-10-methoxy-2,2,4-trimethyl-5H-chromeno(3,4-f)quinoline (compound 158);
(Z)-5-(2′-Hydroxymethylbenzylidene)1,2-dihydro-9-hydroxy-10-methoxy-2,2,4-trimethyl-5H-chromeno(3,4-f)quinoline (compound 159);
(Z)-5-(2′-(3′-(Hydroxymethyl)-5′-methylfuranylmethylidene))1,2-dihydro-9-hydroxy-10-methoxy-2,2,4-trimethyl-5H-chromeno(3,4-f)quinoline (compound 160);
(Z)-5-(2′-Fluoro-3′-hydroxymethylbenzylidene)1,2-dihydro-9-hydroxy-10-methoxy-2,2,4-trimethyl-5H-chromeno(3,4-f)quinoline (compound 161);
(Z)-5-(4′-Fluoro-2′-hydroxymethylbenzylidene)1,2-dihydro-9-hydroxy-10-methoxy-2,2,4-trimethyl-5H-chromeno(3,4-f)quinoline (compound 162);
(Z)-5-(3′-Bromo-2′-hydroxymethylbenzylidene)1,2-dihydro-9-hydroxy-10-methoxy-2,2,4-trimethyl-5H-chromeno(3,4-f)quinoline (compound 163);
(Z)-5-(5′-Bromo-2′-hydroxymethylbenzylidene)1,2-dihydro-9-hydroxy-10-methoxy-2,2,4-trimethyl-5H-chromeno(3,4-f)quinoline (compound 164);
(Z)-5-(2′-(3′-(Piperidinylmethyl)thienylmethylidene))1,2-dihydro-9-hydroxy-10-methoxy-2,2,4-trimethyl-5H-chromeno(3,4-f)quinoline (compound 165);
(Z)-5-(2′-(3′-(Dimethylaminomethyl)thienylmethylidene))1,2-dihydro-9-hydroxy-10-methoxy-2,2,4-trimethyl-5H-chromeno(3,4-f)quinoline (compound 166);
(Z)-5-(2′-(Diethylaminomethyl)-4′-fluorobenzylidene)1,2-dihydro-9-hydroxy-10-methoxy-2,2,4-trimethyl-5H-chromeno(3,4-f)quinoline (compound 167);
(Z)-5-(2′-(3′-Acetylthienylmethylidene))1,2-dihydro-9-hydroxy-10-methoxy-2,2,4-trimethyl-5H-chromeno(3,4-f)quinoline (compound 168);
(Z)-5-(2′-(3′-(1″-Hydroxy-1″-methylethyl)thienylmethylidene))1,2-dihydro-9-hydroxy-10-methoxy-2,2,4-trimethyl-5H-chromeno(3,4-f)quinoline (compound 169);
(Z)-5-(2′-(3′-Benzoylthienylmethylidene))1,2-dihydro-9-hydroxy-10-methoxy-2,2,4-trimethyl-5H-chromeno(3,4-f)quinoline (compound 170);
(±)-(Z)-5-(2′-(3′-(1″-Hydroxyethyl)thienylmethylidene))1,2-dihydro-9-hydroxy-10-methoxy-2,2,4-trimethyl-5H-chromeno(3,4-f)quinoline (compound 171);
(±)-(Z)-5-(2′-(3′-(1″-Hydroxy-1″-phenylmethyl)thienylmethylidene))1,2-dihydro-9-hydroxy-10-methoxy-2,2,4-trimethyl-5H-chromeno(3,4-f)quinoline (compound 172);
(Z)-5-(4′-Fluoro-2′-acetylbenzylidene)1,2-dihydro-9-hydroxy-10-methoxy-2,2,4-trimethyl-5H-chromeno(3,4-f)quinoline (compound 173);
(Z)-5-(2′-(3′-((E)-1″-Hydroxyiminoethyl)thienylmethylidene))1,2-dihydro-9-hydroxy-10-methoxy-2,2,4-trimethyl-5H-chromeno(3,4-f)quinoline (compound 174);
(Z)-5-(2′-(3′-((Z)-1″-Hydroxyiminoethyl)thienylmethylidene))1,2-dihydro-9-hydroxy-10-methoxy-2,2,4-trimethyl-5H-chromeno(3,4-f)quinoline (compound 175);
(Z)-5-(2′-(3′-((E)-1″-Methoxyiminoethyl)thienylmethylidene))1,2-dihydro-9-hydroxy-10-methoxy-2,2,4-trimethyl-5H-chromeno(3,4-f)quinoline (compound 176);
(Z)-5-(2′-(3′-((Z)-1″-Methoxyiminoethyl)thienylmethylidene))1,2-dihydro-9-hydroxy-10-methoxy-2,2,4-trimethyl-5H-chromeno(3,4-f)quinoline (compound 177);
(Z)-5-(2′-(3′-((E)-1″-Allyloxyiminoethyl)thienylmethylidene))1,2-dihydro-9-hydroxy-10-methoxy-2,2,4-trimethyl-5H-chromeno(3,4-f)quinoline (compound 178);
(Z)-5-(2′-(3′-((Z)-1″-Allyloxyiminoethyl)thienylmethylidene))1,2-dihydro-9-hydroxy-10-methoxy-2,2,4-trimethyl-5H-chromeno(3,4-f)quinoline (compound 179);
(Z)-5-(2′-(3′-((E)-1″-Phenoxyiminoethyl)thienylmethylidene))1,2-dihydro-9-hydroxy-10-methoxy-2,2,4-trimethyl-5H-chromeno(3,4-f)quinoline (compound 180);
(Z)-5-(2′-(3′-((Z)-1″-Phenoxyiminoethyl)thienylmethylidene))1,2-dihydro-9-hydroxy-10-methoxy-2,2,4-trimethyl-5H-chromeno(3,4-f)quinoline (compound 181);
(Z)-5-(2′-(3′-((E)-1″-Ethoxyiminoethyl)thienylmethylidene))1,2-dihydro-9-hydroxy-10-methoxy-2,2,4-trimethyl-5H-chromeno(3,4-f)quinoline (compound 182);
(Z)-5-(2′-(3′-((Z)-1″-Ethoxyiminoethyl)thienylmethylidene))1,2-dihydro-9-hydroxy-10-methoxy-2,2,4-trimethyl-5H-chromeno(3,4-f)quinoline (compound 183);
(Z)-5-(2′-(3′-((E)-(Carboxymethoxy)iminoethyl)thienylmethylidene))1,2-dihydro-9-hydroxy-10-methoxy-2,2,4-trimethyl-5H-chromeno(3,4-f)quinoline (compound 184);
(Z)-5-(2′-(3′-((E)-1″-tert-Butoxyiminoethyl)thienylmethylidene))1,2-dihydro-9-hydroxy-10-methoxy-2,2,4-trimethyl-5H-chromeno(3,4-f)quinoline (compound 185);
(Z)-5-(2′-(3′-((E)-1″-Benzyloxyiminoethyl)thienylmethylidene))1,2-dihydro-9-hydroxy-10-methoxy-2,2,4-trimethyl-5H-chromeno(3,4-f)quinoline (compound 186);
(Z)-5-(2′-(3′-((Z)-1″-Benzyloxyiminoethyl)thienylmethylidene))1,2-dihydro-9-hydroxy-10-methoxy-2,2,4-trimethyl-5H-chromeno(3,4-f)quinoline (compound 187);
(Z)-5-(2′-(3′-((E)-1″-(p-Nitrobenzyloxy)iminoethyl)thienylmethylidene))1,2-dihydro-9-hydroxy-10-methoxy-2,2,4-trimethyl-5H-chromeno(3,4-f)quinoline (compound 188);
(Z)-5-(2′-(3′-((Z)-1″-(p-Nitrobenzyloxy)iminoethyl)thienylmethylidene))1,2-dihydro-9-hydroxy-10-methoxy-2,2,4-trimethyl-5H-chromeno(3,4-f)quinoline (compound 189);
(Z)-5-(2′-(3′-((E)-Hydroxyiminomethyl)thienylmethylidene))1,2-dihydro-9-hydroxy-10-methoxy-2,2,4-trimethyl-5H-chromeno(3,4-f)quinoline (compound 190);
(Z)-5-(4′-Fluoro-(E)-2′-(hydroxyiminomethyl)benzylidene)1,2-dihydro-9-hydroxy-10-methoxy-2,2,4-trimethyl-5H-chromeno(3,4-f)quinoline (compound 191);
(Z)-5-(2′-(Hydroxyiminomethyl)benzylidene)1,2-dihydro-9-hydroxy-10-methoxy-2,2,4-trimethyl-5H-chromeno(3,4-f)quinoline (compound 192);
(Z)-5-(2′-(3′-Methoxymethylthienylmethylidene))1,2-dihydro-9-hydroxy-10-methoxy-2,2,4-trimethyl-5H-chromeno(3,4-f)quinoline (compound 193);
(Z)-5-(2′-(3′-(Methoxymethoxymethyl)thienylmethylidene))1,2-dihydro-9-hydroxy-10-methoxy-2,2,4-trimethyl-5H-chromeno(3,4-f)quinoline (compound 194);
(Z)-5-(2′-(3′-Prop-2″-enyloxymethylthienylmethylidene))1,2-dihydro-9-hydroxy-10-methoxy-2,2,4-trimethyl-5H-chromeno(3,4-f)quinoline (compound 195);
(Z)-5-(2′-(3′-(Prop-2″-ynloxymethyl)thienylmethylidene))1,2-dihydro-9-hydroxy-10-methoxy-2,2,4-trimethyl-5H-chromeno(3,4-f)quinoline (compound 196);
(Z)-5-(4′-Fluoro-2′-(methoxymethoxymethyl)benzylidene)1,2-dihydro-9-hydroxy-10-methoxy-2,2,4-trimethyl-5H-chromeno(3,4-f)quinoline (compound 197);
(Z)-5-(2′-(Methoxymethoxymethyl)benzylidene)1,2-dihydro-9-hydroxy-10-methoxy-2,2,4-trimethyl-5H-chromeno(3,4-f)quinoline (compound 198);
(±)-(Z)-5-(2′-(3′-(1″-Hydroxybut-3″-enyl)thienylmethylidene))1,2-dihydro-9-hydroxy-10-methoxy-2,2,4-trimethyl-5H-chromeno(3,4-f)quinoline (compound 199);
(+)-(Z)-5-(2′-(3′-(1″-Hydroxybut-3″-enyl)thienylmethylidene))1,2-dihydro-9-hydroxy-10-methoxy-2,2,4-trimethyl-5H-chromeno(3,4-f)quinoline (compound 200);
(−)-(Z)-5-(2′-(3′-(1″-Hydroxybut-3″-enyl)thienylmethylidene))1,2-dihydro-9-hydroxy-10-methoxy-2,2,4-trimethyl-5H-chromeno(3,4-f)quinoline (compound 201);
(±)-(Z)-5-(2′-(3′-(1″-Hydroxy-2″,2″,2″-trifluoroethyl)thienylmethylidene))1,2-dihydro-9-hydroxy-10-methoxy-2,2,4-trimethyl-5H-chromeno(3,4-f)quinoline (compound 202);
(+)-(Z)-5-(2′-(3′-(1″-Hydroxy-2″,2″,2″-trifluoroethyl)thienylmethylidene))1,2-dihydro-9-hydroxy-10-methoxy-2,2,4-trimethyl-5H-chromeno(3,4-f)quinoline (compound 203);
(−)-(Z)-5-(2′-(3′-(1″-Hydroxy-2″,2″,2″-trifluoroethyl)thienylmethylidene))1,2-dihydro-9-hydroxy-10-methoxy-2,2,4-trimethyl-5H-chromeno(3,4-f)quinoline (compound 204);
(±)-(Z)-5-(2′-(3′-(1″-Hydroxyprop-2″-ynyl)thienylmethylidene))1,2-dihydro-9-hydroxy-10-methoxy-2,2,4-trimethyl-5H-chromeno(3,4-f)quinoline (compound 205);
(±)-(Z)-5-(4′-fluoro-2′-(2″,2″,2″-Trifluoro-1″-hydroxyethyl)benzylidene)1,2-dihydro-9-hydroxy-10-methoxy-2,2,4-trimethyl-5H-chromeno(3,4-f)quinoline (compound 206);
(±)-(Z)-5-(2′-(3′-(Hydroxythien-3″-ylmethyl)thienylmethylidene))1,2-dihydro-9-hydroxy-10-methoxy-2,2,4-trimethyl-5H-chromeno(3,4-f)quinoline (compound 207);
(±)-(Z)-5-(2′-(3′-((4″-Fluorophenyl)hydroxymethyl)thienylmethylidene))1,2-dihydro-9-hydroxy-10-methoxy-2,2,4-trimethyl-5H-chromeno(3,4-f)quinoline (compound 208);
(±)-(Z)-5-(2′-(3′-(1″-Hydroxyallyl)thienylmethylidene))1,2-dihydro-9-hydroxy-10-methoxy-2,2,4-trimethyl-5H-chromeno(3,4-f)quinoline (compound 209);
(±)-(Z)-5-(2′-(3′-(Cyclohexylhydroxymethyl)thienylmethylidene))1,2-dihydro-9-hydroxy-10-methoxy-2,2,4-trimethyl-5H-chromeno(3,4-f)quinoline (compound 210);
(±)-(Z)-5-(2′-(3′-(1″-Hydroxy-2″-phenylethyl)thienylmethylidene))1,2-dihydro-9-hydroxy-10-methoxy-2,2,4-trimethyl-5H-chromeno(3,4-f)quinoline (compound 211);
(±)-(Z)-5-(2′-(3′-(Hydroxythien-2″-ylmethyl)thienylmethylidene))1,2-dihydro-9-hydroxy-10-methoxy-2,2,4-trimethyl-5H-chromeno(3,4-f)quinoline (compound 212);
(Z)-5-(2′-(3′-Acryloylthienylmethylidene))1,2-dihydro-9-hydroxy-10-methoxy-2,2,4-trimethyl-5H-chromeno(3,4-f)quinoline (compound 213);
(Z)-5-(2′-(3′-(4″-Fluorobenzoyl)thienylmethylidene))1,2-dihydro-9-hydroxy-10-methoxy-2,2,4-trimethyl-5H-chromeno(3,4-f)quinoline (compound 214);
(Z)-5-(2′-(3″-(Thien-3″-ylcarbonyl)thienylmethylidene))1,2-dihydro-9-hydroxy-10-methoxy-2,2,4-trimethyl-5H-chromeno(3,4-f)quinoline (compound 215);
(Z)-5-(2′-(3′-(Cyclohexanecarbonyl)thienylmethylidene))1,2-dihydro-9-hydroxy-10-methoxy-2,2,4-trimethyl-5H-chromeno(3,4-f)quinoline (compound 216);
(Z)-5-(2′-(3′-(But-3″-enoyl)thienylmethylidene))1,2-dihydro-9-hydroxy-10-methoxy-2,2,4-trimethyl-5H-chromeno(3,4-f)quinoline (compound 217);
(Z)-5-(2′-(3′-(Aminomethyl)thienylmethylidene))1,2-dihydro-9-hydroxy-10-methoxy-2,2,4-trimethyl-5H-chromeno(3,4-f)quinoline (compound 218);
(Z)-5-(2′-(3′-(Phenylaminomethyl)thienylmethylidene))1,2-dihydro-9-hydroxy-10-methoxy-2,2,4-trimethyl-5H-chromeno(3,4-f)quinoline (compound 219);
(Z)-5-(2′-(3′-(Prop-2″-ynylaminomethyl)thienylmethylidene))1,2-dihydro-9-hydroxy-10-methoxy-2,2,4-trimethyl-5H-chromeno(3,4-f)quinoline (compound 220);
(Z)-5-(2′-(3′-((2″,2″,2″-Trifluoroethylamino)methyl)thienylmethylidene))1,2-dihydro-9-hydroxy-10-methoxy-2,2,4-trimethyl-5H-chromeno(3,4-f)quinoline (compound 221);
(Z)-5-(2′-(3′-(Cyclopropylaminomethyl)thienylmethylidene))1,2-dihydro-9-hydroxy-10-methoxy-2,2,4-trimethyl-5H-chromeno(3,4-f)quinoline (compound 222);
(Z)-5-(2′-(3′-(1″-Butylaminomethyl)thienylmethylidene))1,2-dihydro-9-hydroxy-10-methoxy-2,2,4-trimethyl-5H-chromeno(3,4-f)quinoline (compound 223);
(Z)-5-(2′-(3′-(2″-Hydroxyethoxymethyl)thienylmethylidene))1,2-dihydro-9-hydroxy-10-methoxy-2,2,4-trimethyl-5H-chromeno(3,4-f)quinoline (compound 224);
(Z)-5-(2′-(3′-Isopropenylthienylmethylidene))1,2-dihydro-9-hydroxy-10-methoxy-2,2,4-trimethyl-5H-chromeno(3,4-f)quinoline (compound 225);
(Z)-5-(2′-(3′-Formylthienylmethylidene))1,2-dihydro-9-hydroxy-10-methoxy-2,2,4-trimethyl-5H-chromeno(3,4-f)quinoline (compound 226);
(Z)-5-(2′-(3′-(Methoxyethoxymethoxymethyl)thienylmethylidene))1,2-dihydro-9-hydroxy-10-methoxy-2,2,4-trimethyl-5H-chromeno(3,4-f)quinoline (compound 227);
(Z)-5-(2′-(3′-(Trifluoroacetyl)thienylmethylidene))1,2-dihydro-9-hydroxy-10-methoxy-2,2,4-trimethyl-5H-chromeno(3,4-f)quinoline (compound 228);
(Z)-5-(2′-(3′-(2″,2″,2″-Trifluoro-1″-hydroxy-1″-(trifluoromethyl)ethyl)thienylmethylidene))1,2-dihydro-9-hydroxy-10-methoxy-2,2,4-trimethyl-5H-chromeno(3,4-f)quinoline (compound 229);
(Z)-5-(4′-Fluoro-2′-formylbenzylidene)1,2-dihydro-9-hydroxy-10-methoxy-2,2,4-trimethyl-5H-chromeno(3,4-f)quinoline (compound 230);
(Z)-5-(2′-(3′-Cyanothienylmethylidene))1,2-dihydro-9-hydroxy-10-methoxy-2,2,4-trimethyl-5H-chromeno(3,4-f)quinoline (compound 231);
(Z)-5-(2′-(3′-Carbamoylthienylmethylidene))1,2-dihydro-9-hydroxy-10-methoxy-2,2,4-trimethyl-5H-chromeno(3,4-f)quinoline (compound 232);
(Z)-5-(4′-Fluoro-2′-vinylbenzylidene)1,2-dihydro-9-hydroxy-10-methoxy-2,2,4-trimethyl-5H-chromeno(3,4-f)quinoline (compound 233);
(Z)-5-(4′-Fluoro-2′-(acetoxymethyl)benzylidene)1,2-dihydro-9-hydroxy-10-methoxy-2,2,4-trimethyl-5H-chromeno(3,4-f)quinoline (compound 234);
(Z)-5-(2′-Formylbenzylidene)1,2-dihydro-9-hydroxy-10-methoxy-2,2,4-trimethyl-5H-chromeno(3,4-f)quinoline (compound 235);
(Z)-5-(2′-Vinylbenzylidene)1,2-dihydro-9-hydroxy-10-methoxy-2,2,4-trimethyl-5H-chromeno(3,4-f)quinoline (compound 236);
(Z)-5-(2′-(3′-(But-2″-ynloxymethyl)thienylmethylidene))1,2-dihydro-9-hydroxy-10-methoxy-2,2,4-trimethyl-5H-chromeno(3,4-f)quinoline (compound 237);
(Z)-5-(2′-(3′-(2″-(E)-Cyanovinyl)thienylmethylidene))1,2-dihydro-9-hydroxy-10-methoxy-2,2,4-trimethyl-5H-chromeno(3,4-f)quinoline (compound 238);
(Z)-5-(2′-(3′-(Ethoxycarbonylmethoxymethyl)thienylmethylidene))1,2-dihydro-9-hydroxy-10-methoxy-2,2,4-trimethyl-5H-chromeno(3,4-f)quinoline (compound 239);
(Z)-5-(2′-(3′-(Carboxymethoxymethyl)thienylmethylidene))1,2-dihydro-9-hydroxy-10-methoxy-2,2,4-trimethyl-5H-chromeno(3,4-f)quinoline (compound 240);
(Z)-5-(2′-(3′-Vinylthienylmethylidene))1,2-dihydro-9-hydroxy-10-methoxy-2,2,4-trimethyl-5H-chromeno(3,4-f)quinoline (compound 241);
(±)-(Z)-5-(2′-(3′-(1″-Methoxy-2″,2″,2″-trifluoroethyl)thienylmethylidene))1,2-dihydro-9-hydroxy-10-methoxy-2,2,4-trimethyl-5H-chromeno(3,4-f)quinoline (compound 242);
(Z)-5-(2′-(3′-(2″,2″,2″-Trifluoro-1″-methoxy-1″-(trifluoromethyl)ethyl)thienylmethylidene))1,2-dihydro-9-hydroxy-10-methoxy-2,2,4-trimethyl-5H-chromeno(3,4-f)quinoline (compound 243);
(Z)-5-(4′-Hydroxymethylbenzylidene)1,2-dihydro-9-hydroxy-10-methoxy-2,2,4-trimethyl-5H-chromeno(3,4-f)quinoline (compound 244);
(Z)-5-(2′-(3′-(1″-Hydroxy-1″-(thien-3″-yl)ethyl)thienylmethylidene))1,2-dihydro-9-hydroxy-10-methoxy-2,2,4-trimethyl-5H-chromeno(3,4-f)quinoline (compound 245);
(Z)-5-(2′-(3′-(2″-Methoxycarbonylvinyl)thienylmethylidene))1,2-dihydro-9-hydroxy-10-methoxy-2,2,4-trimethyl-5H-chromeno(3,4-f)quinoline (compound 246);
(Z)-5-(2′-(3′-Hydroxymethylpyridinylmethylidene))1,2-dihydro-9-hydroxy-10-methoxy-2,2,4-trimethyl-5H-chromeno(3,4-f)quinoline (compound 247);
(Z)-5-(2′-(3′-(Hydroxyethylcarbamoyl)thienylmethylidene))1,2-dihydro-9-hydroxy-10-methoxy-2,2,4-trimethyl-5H-chromeno(3,4-f)quinoline (compound 248);
(Z)-5-(2′-(3′-Ethylcarbamoylthienylmethylidene))1,2-dihydro-9-hydroxy-10-methoxy-2,2,4-trimethyl-5H-chromeno(3,4-f)quinoline (compound 249);
(Z)-5-(2′-(3′-((R)-2″-(Carbomethoxy)pyrrolidinecarbonyl)thienylmethylidene))1,2-dihydro-9-hydroxy-10-methoxy-2,2,4-trimethyl-5H-chromeno(3,4-f)quinoline (compound 250);
(Z)-5-(2′-(3′-(Piperazinecarbonyl)thienylmethylidene))1,2-dihydro-9-hydroxy-10-methoxy-2,2,4-trimethyl-5H-chromeno(3,4-f)quinoline (compound 251);
(Z)-5-(2′-(3′-(4″-Oxo-piperidinecarbonyl)thienylmethylidene))1,2-dihydro-9-hydroxy-10-methoxy-2,2,4-trimethyl-5H-chromeno(3,4-f)quinoline (compound 252);
(Z)-5-(2′-(3′-(2″,2″,2″-Trifluoroethylcarbamoyl)thienylmethylidene))1,2-dihydro-9-hydroxy-10-methoxy-2,2,4-trimethyl-5H-chromeno(3,4-f)quinoline (compound 253);
(Z)-5-(2′-(3′-(4″-Hydroxypiperidinecarbonyl)thienylmethylidene))1,2-dihydro-9-hydroxy-10-methoxy-2,2,4-trimethyl-5H-chromeno(3,4-f)quinoline (compound 254);
(Z)-5-(2′-(3′-(4″-Methylpiperazinecarbonyl)thienylmethylidene))1,2-dihydro-9-hydroxy-10-methoxy-2,2,4-trimethyl-5H-chromeno(3,4-f)quinoline (compound 256);
(±)-(Z)-5-(2′-(3′-(1″-Hydroxy-4″,4″,4″-trifluorobut-2″-ynyl)thienylmethylidene))1,2-dihydro-9-hydroxy-10-methoxy-2,2,4-trimethyl-5H-chromeno[3,4-f]quinoline (compound 257);
(Z)-5-(2′-(3′-(3″-Hydroxy-3″-phenylpropanoyl)thienylmethylidene))1,2-dihydro-9-hydroxy-10-methoxy-2,2,4-trimethyl-5H-chromeno[3,4-f]quinoline (compound 258);
(Z)-5-(2′-(3″-(3′″-Hydroxybutanoyl)thienylmethylidene))1,2-dihydro-9-hydroxy-10-methoxy-2,2,4-trimethyl-5H-chromeno(3,4-f)quinoline (compound 259); and
(Z)-5-(2′-(3′-(But-2″-enoyl)thienylmethylidene))1,2-dihydro-9-hydroxy-10-methoxy-2,2,4-trimethyl-5H-chromeno(3,4-f)quinoline (compound 260).

Certain compounds provided herein can exist as stereoisomers including optical isomers. All stereoisomers and both the racemic mixtures of such stereoisomers as well as the individual enantiomers that can be separated according to methods that are known in the art are contemplated herein.

C. Preparation of the Compounds

In certain embodiments, synthesis of compounds provided herein is accomplished using Scheme I.

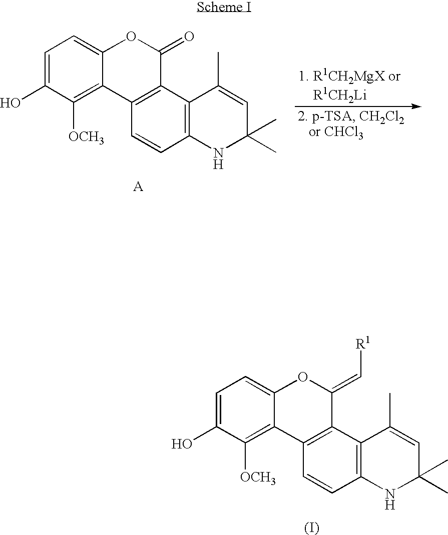

Certain schemes for synthesizing a compound having structure A have been previously discussed. See e.g., U.S. Pat. No. 6,506,766. The process of Scheme I involves the addition of an organometallic reagent, for example an organomagnesium or organolithium reagent, to the compound of structure A. Dehydration of the intermediate with an acid, such as p-toluenesulfonic acid, affords compounds of the generic Formula I.

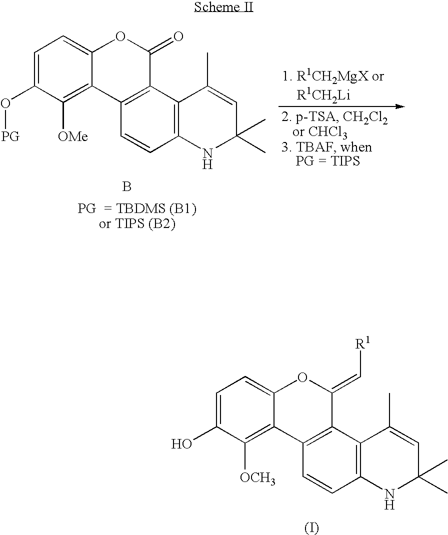

The process of Scheme II begins with the addition of an organometallic reagent, for example, a N,N-diethyl-2-(lithiomethyl)benzamide, to a lactone, for example 9-(tert-butyldimethylsilyl)oxy-10-methoxy-2,2,4-trimethyl-1,2-dihydro-5H-chromeno[3,4-f]quinoline-5-one, to afford a lactol. The organometallic reagent is derived from an arylmethyl or heteroarylmethyl derivative. The lactol is dehydrated to the corresponding olefin by treatment with an acid, for example, p-toluenesulfonic acid, to afford the corresponding benzylidene or heteroarylmethylidene of Structure I. Alternatively, if the protecting group is stable to acidic conditions, the deprotection can take place in a separate operation, for example, when a triisopropylsilyl protecting group is used (B2, Scheme II), a compound of Structure I is formed by treatment with a fluoride source, for example, tetrabutylammonium fluoride (TBAP). Other protecting groups, for example a methoxymethyl ether (40M) group can be employed in the addition-dehydration process.

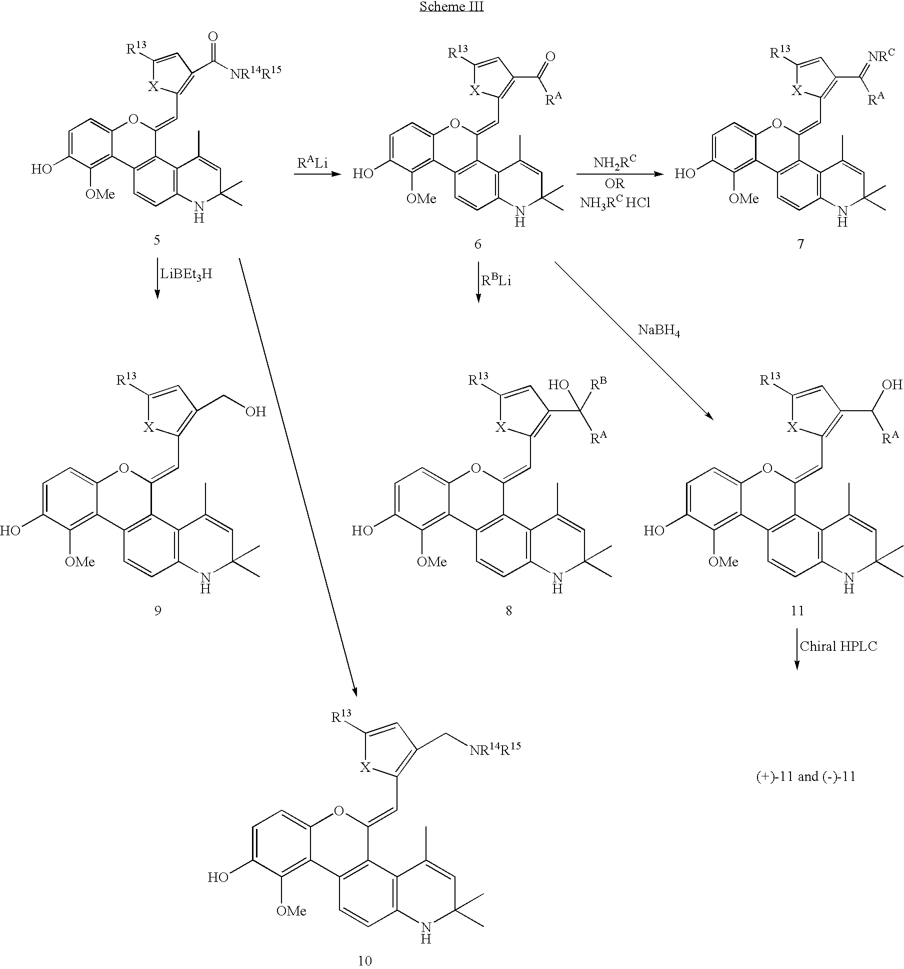

A processes to form compounds of Structures 6, 7, 8, 9, 10, and 11 are depicted in Scheme III. Treatment of Structure 5 with an organometallic reagent, for example, excess methyllithium, affords a compound of Structure 6. Structure 6 can be treated with an amine derivative, for example, hydroxylamine hydrochloride, to afford a compound of Structure 7. Compounds of Structure 7 can form as either the E- or Z-isomer, or as a mixture of both isomers that can be separated by column chromatography or HPLC. Structure 6 can be treated with additional organometallic reagent, for example, phenyllithium, to afford a compound of Structure 8. Structure 5 can be treated with a reducing agent, for example, excess lithium triethylborohydride, to afford the alcohol compound of Structure 9. The amide of Structure 5 can be reduced to the corresponding amine by treatment with certain reducing agents, for example, sequential treatment with alane then sodium cyanoborohydride in acetic acid, to afford a compound of Structure 10. A compound of Structure 6 can be reduced to the corresponding alcohol by treatment with a reducing agent, for example, sodium borohydride, to afford a compound of Structure 11. Asymmetrically pure derivatives of Structure 11 can be obtained by chiral HPLC separation, using, for example, a Chiracel OD column, to afford compounds of Structure (+)-11 and (−)-11. Alternatively, it could be obtained by an asymmetric reduction of structure 6 using an external chiral reagent, for example, an asymmetric CBZ reduction to afford either (+)-11 or 11. Racemic derivatives of Structure 8 can be separated into their enantiomerically pure forms by chiral HPLC using, for example, a Chiracel OD column, to afford compounds of Structure (+)-8- or (−)-8.

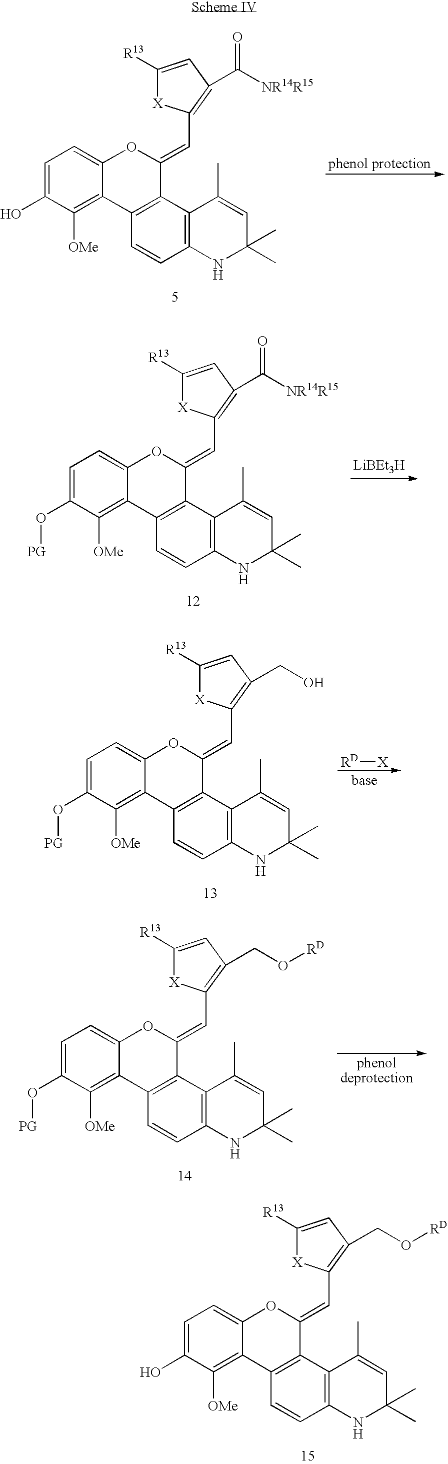

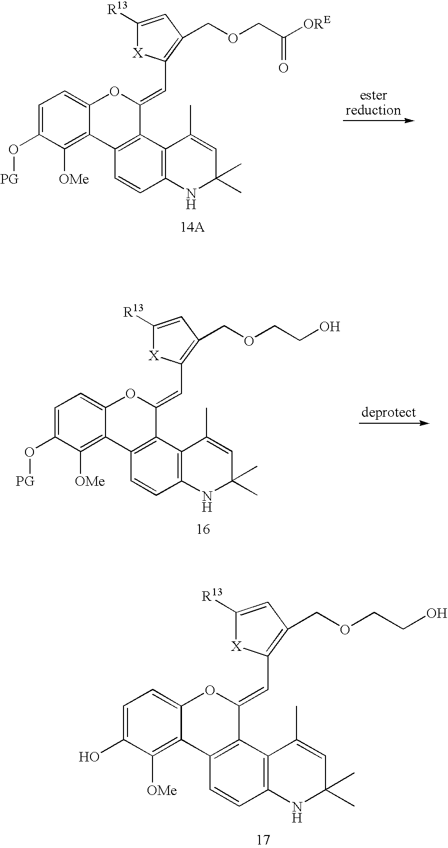

A process to form a compound of Structure 15 is shown in Scheme IV. Structure 5 is converted to the corresponding protected phenol derivative, by treatment with, for example, TIPS-OTf, to afford a compound of Structure 12. Structure 12 is treated with a reducing agent, for example, lithium triethylborohydride, to afford a compound of Structure 13. Structure 13 is alkylated by treatment with an alkylating agent, for example, allyl bromide, to afford a compound of Structure 14. Phenol deprotection is accomplished by treatment with the appropriate deprotection group. For example, when a TIPS protecting group is used (PG=triisopropylsilyl), a fluoride source, for example, TBAF, can be used to afford a compound of Structure 15. If an acid-sensitive protecting group is used, for example, a MOM ether, deprotection can be performed by treatment with an acid, for example hydrochloric acid. The synthesis of a compound of Structure 17 is depicted in Scheme IV. Structure 13 is treated with an alkylating agent, for example, ethyl bromoacetate, and a base, for example, potassium carbonate, to afford a compound of Structure 14A. Compound 14A is reduced to the corresponding alcohol by treatment with a reducing agent, for example, sodium borohydride, to afford a compound of Structure 16. Phenol deprotection is accomplished by treatment with the appropriate deprotection group. For example, when a TIPS protecting group is used (PG=triisopropylsilyl), a fluoride source, for example, TBAF, can be used to afford a compound of Structure 17.

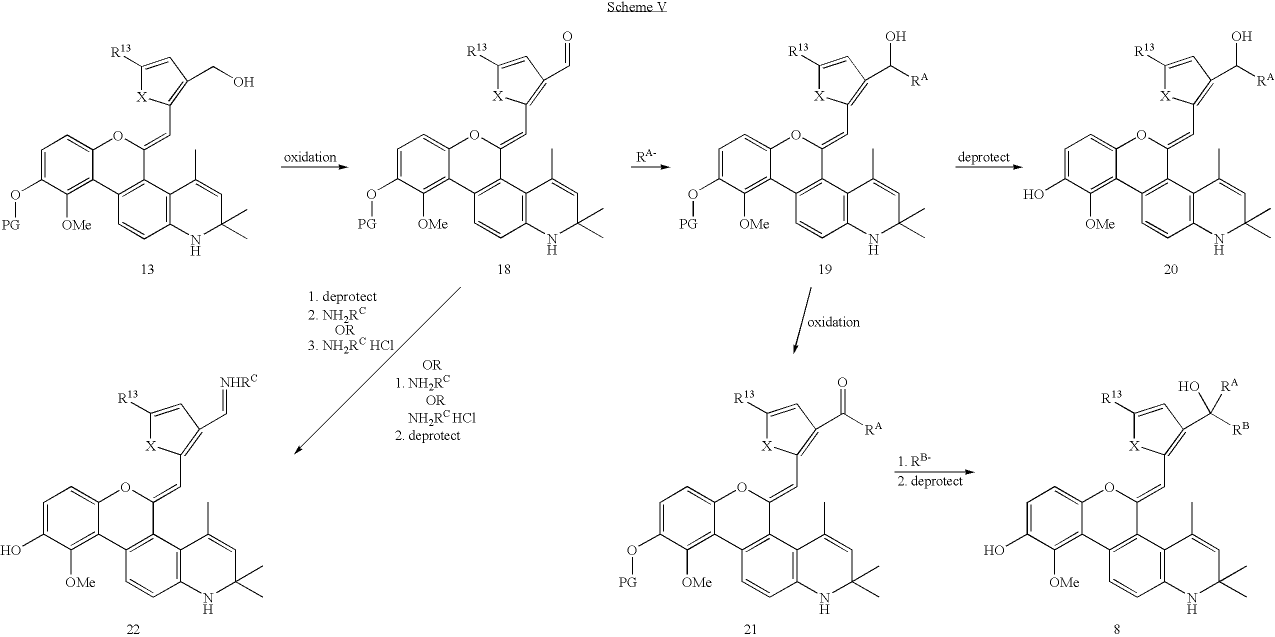

A processes to form compounds of Structures 20, 8, and 22 are depicted in Scheme V. Structure 13 is treated with an oxidizing agent, for example, 1-hydroxy 1,2-benziodoxal-3(1H)-one-1-oxide (IBX), to afford a compound of Structure 18.

Treatment of 18 with a carbon nucleophile, for example, phenyl magnesium bromide, affords a compound of Structure 19. Phenol deprotection is accomplished by treatment with the appropriate deprotection group. For example, when the TIPS protecting group is used (PG=triisopropylsilyl), a fluoride source, for example, TBAF, can be used to afford a compound of Structure 20. The preparation of a compound of Structure 8 can be accomplished by treatment of Structure 19 with an oxidizing agent, for example, 1-hydroxy 1,2-benziodoxal-3(1H)-one-1-oxide (IBX), to afford a compound of Structure 21. Carbonyl addition to Structure 21 can be accomplished by carbon nucleophile, for example, methyllithium, followed by subsequent deprotection under the appropriate conditions, to afford a compound of Structure 8. Compounds of Structure 22 can be prepared by deprotection of the phenol protecting group with an appropriate reagent. For example, when the TIPS protecting group is used, TBAF can be used followed by addition of an amino derivative, for example, hydroxylamine hydrochloride, to afford a compound of Structure 22. Alternatively, the preparation of Structure 22 can proceed from Structure 18 by addition of an amino-derivative, for example, methoxyamine hydrochloride, followed by subsequent deprotection as described above, to afford a compound of Structure 22.

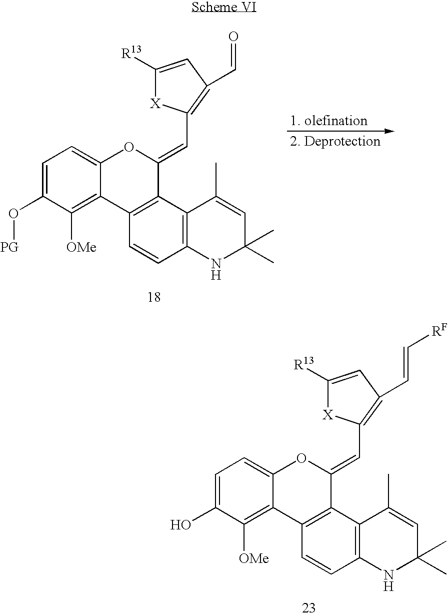

A process to form a compound of Structure 23 is depicted in Scheme VI. Treatment of Structure 18 with an olefinating reagent, for example, Tebbe's reagent, followed by deprotection of the phenol protecting group, affords a compound of Structure 23. Phenol deprotection is accomplished by treatment with the appropriate deprotection group. For example, when the TIPS protecting group is used (PG=triisopropylsilyl), a fluoride source, for example, TBAF, can be used to afford a compound of Structure 23.

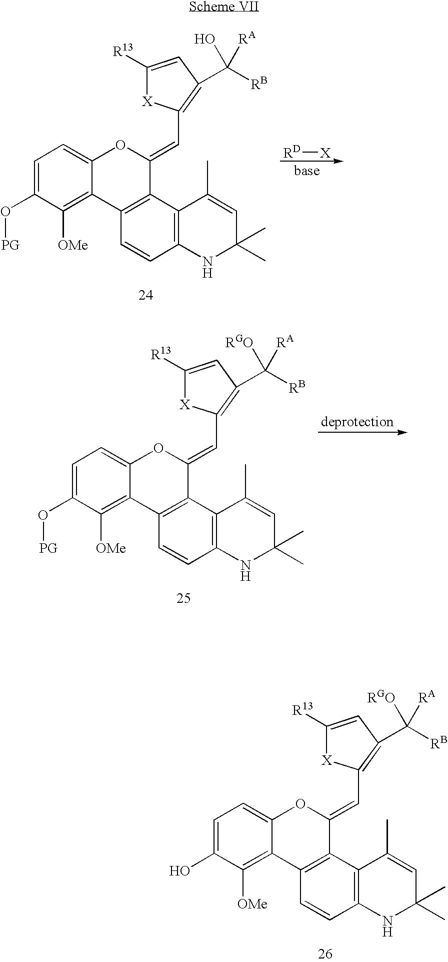

A process to form Structure 26 is depicted in Scheme VII. Alkylation of a compound of Structure 24 can be accomplished by treatment with an alkylating agent, for example, methyl iodide, to afford a compound of Structure 25. Phenol deprotection is accomplished by treatment with the appropriate deprotection group. For example, when the TIPS protecting group is used (PG=triisopropylsilyl), a fluoride source, for example, TBAF, can be used to afford a compound of Structure 26.

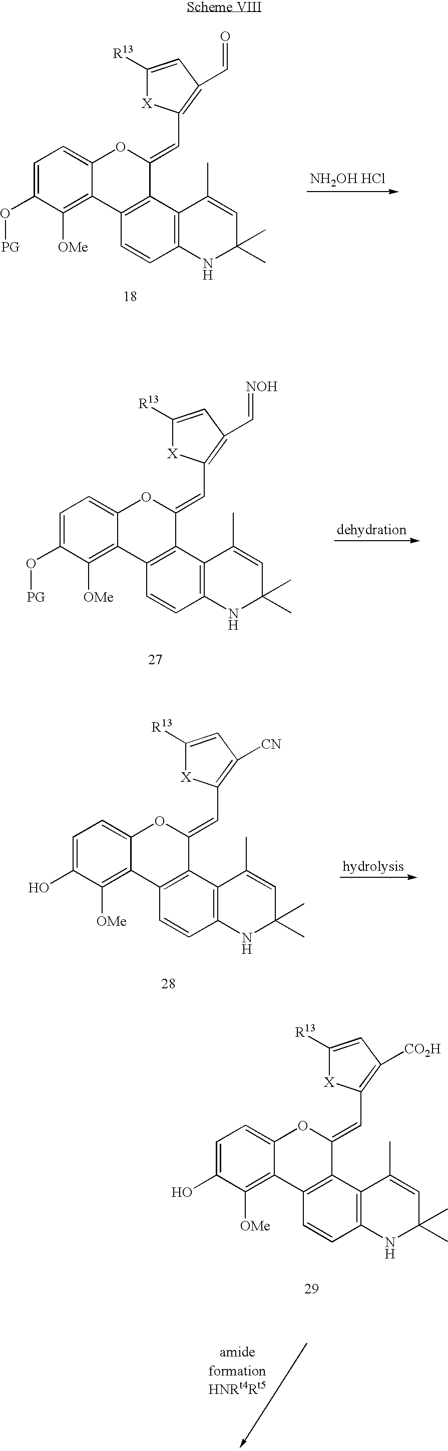

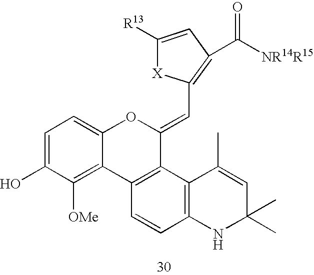

A processes to form compounds of Structure 28, 29, and 30 are depicted in Scheme VIII. Structure 18 can be treated with hydroxylamine hydrochloride to afford a compound of Structure 27. Structure 27 can be dehydrated to the corresponding cyano compound by treatment with, for example, 1,1′-carbonyldiimidazole, to afford a compound of Structure 28. Hydrolysis of Structure 28 to the corresponding carboxylic acid can be effected by hydrolysis with, for example, potassium hydroxide in ethylene glycol at elevated temperatures, to afford a compound of Structure 29. Treatment of Structure 29 with an amine, for example, ethylamine, in the presence of carboxylic acid activating reagents, for example 1-hydroxybenzotriazole hydrate (HOBT) and 1-(3-dimethylaminopropyl)-3-ethylcarbodiimide hydrochloride (EDC), affords a compound of Structure 30.

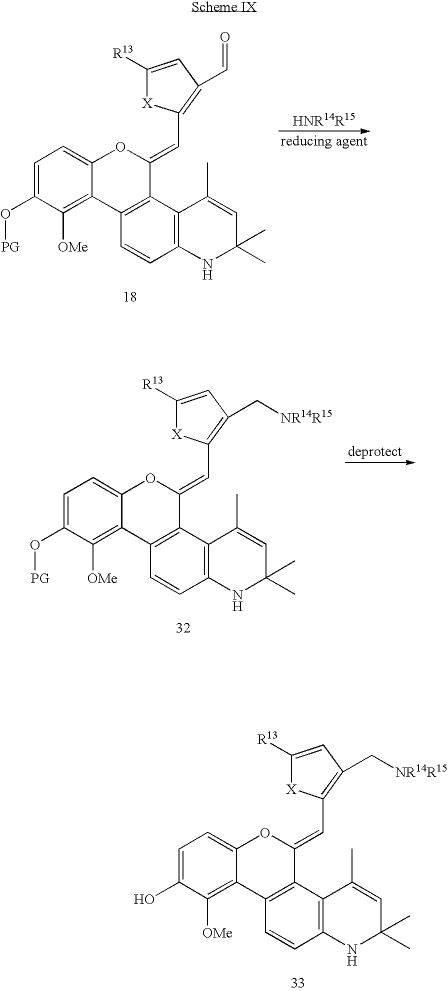

A process to form a compound of Structure 33 is depicted in Scheme IX. Treatment of Structure 18 with an amine, for example benzylamine, in the presence of a reducing agent, for example, sodium cyanoborohydride, affords a compound of Structure 32. Deprotection to the phenol is accomplished by treatment with the appropriate deprotection group. For example, when the TIPS protecting group is used (PG=triisopropylsilyl), a fluoride source, for example, TBAF, can be used to afford a compound of Structure 33.

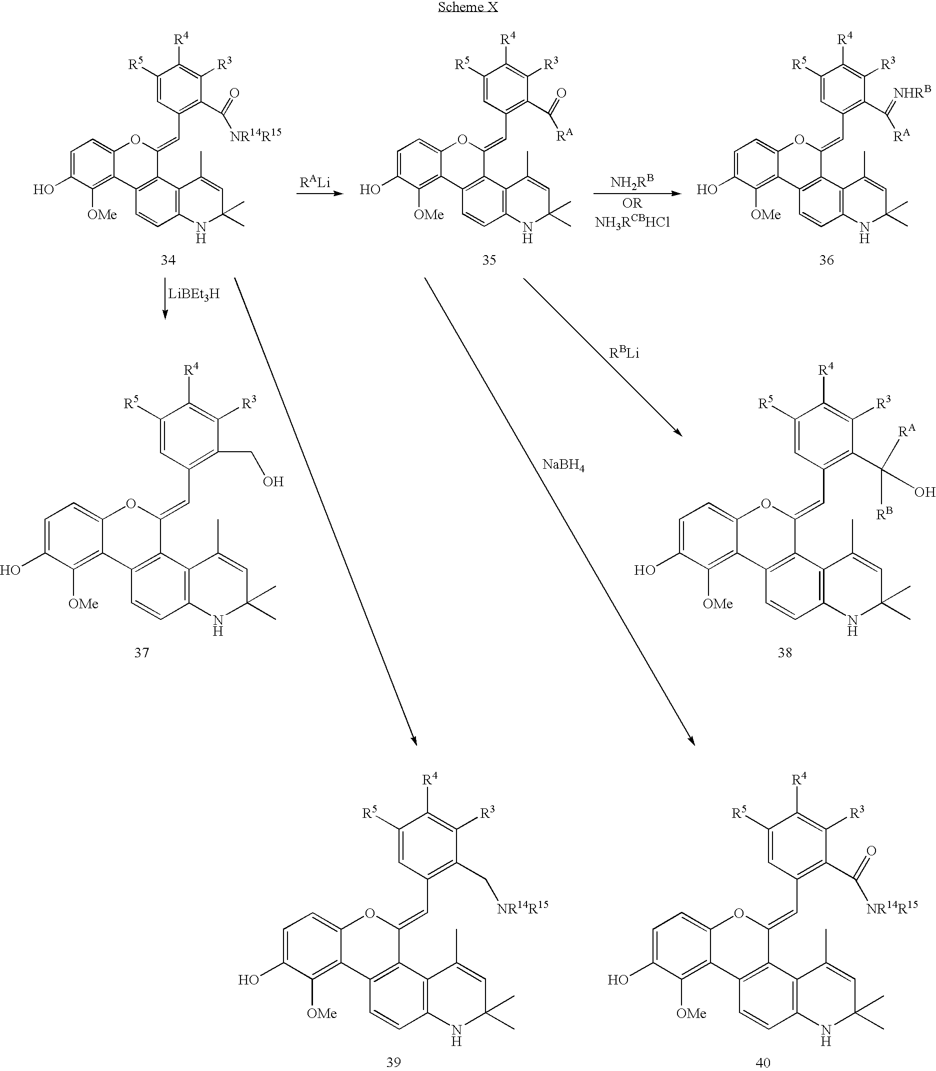

A processes to form compounds of Structures 36, 37, 38, 39, and 40 are depicted in Scheme X. Treatment of Structure 34 with an organometallic reagent, for example, excess methyllithium, affords a compound of Structure 35. Structure 35 can be treated with an amine derivative, for example, hydroxylamine hydrochloride, to afford a compound of Structure 36. Compounds of Structure 36 can form as either the E- or Z-isomer, or as a mixture of both isomers that can be separated by column chromatography or HPLC. Structure 35 can be treated with additional organometallic reagent, for example, phenyllithium, to afford a compound of Structure 38. Structure 34 can be treated with a reducing agent, for example, excess lithium triethylborohydride, to afford the alcohol compound of Structure 37. The amide of Structure 34 can be reduced to the corresponding amine by treatment with certain reducing agents, for example, sequential treatment with alane then sodium cyanoborohydride in acetic acid, to afford a compound of Structure 39. A compound of Structure 35 can be reduced to the corresponding alcohol by treatment with a reducing agent, fox example, sodium borohydride, to afford a compound of Structure 40. Asymmetrically pure derivatives of Structure 40 can be obtained by chiral HPLC separation, using, for example, a Chiracel OD column, to afford compounds of Structure (+)-40 and (−)-40. Alternatively, it could be obtained by an asymmetric reduction of structure 6 using an external chiral reagent, for example, an asymmetric CBZ reduction to afford either (+)-40 or 40. Racemic derivatives of Structure 38 can be separated into their enantiomerically pure forms by chiral HPLC using, for example, a Chiracel OD column, to afford compounds of Structure (+)-38- or (−)-38.

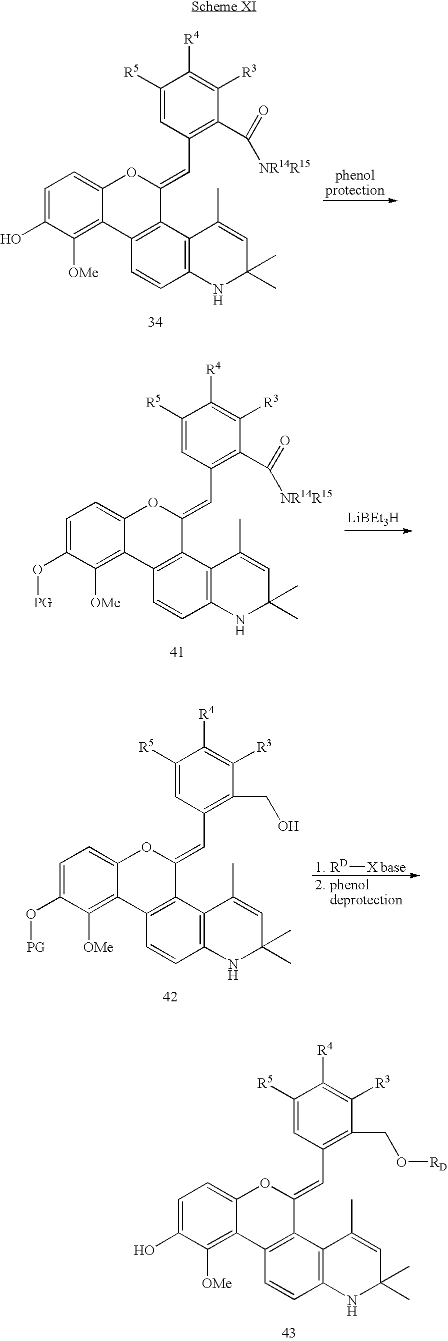

A process to form compounds of Structure 43 is depicted in Scheme XI. Structure 34 is converted to the corresponding protected phenol derivative, by treatment with, for example, TIPS-OTf, to afford a compound of Structure 41. Structure 41 is treated with a reducing agent, for example, lithium triethylborohydride, to afford a compound of Structure 42. Structure 42 is alkylated by treatment with an alkylating agent, for example, allyl bromide, followed by treatment with the appropriate deprotection group. For example, when a TIPS protecting group is used (PG=triisopropylsilyl), a fluoride source, for example, TBAF, can be used to afford a compound of Structure 43. If an acid-sensitive protecting group is used, for example, a MOM ether, deprotection can be performed by treatment with an acid, for example hydrochloric acid to afford Structure 43.

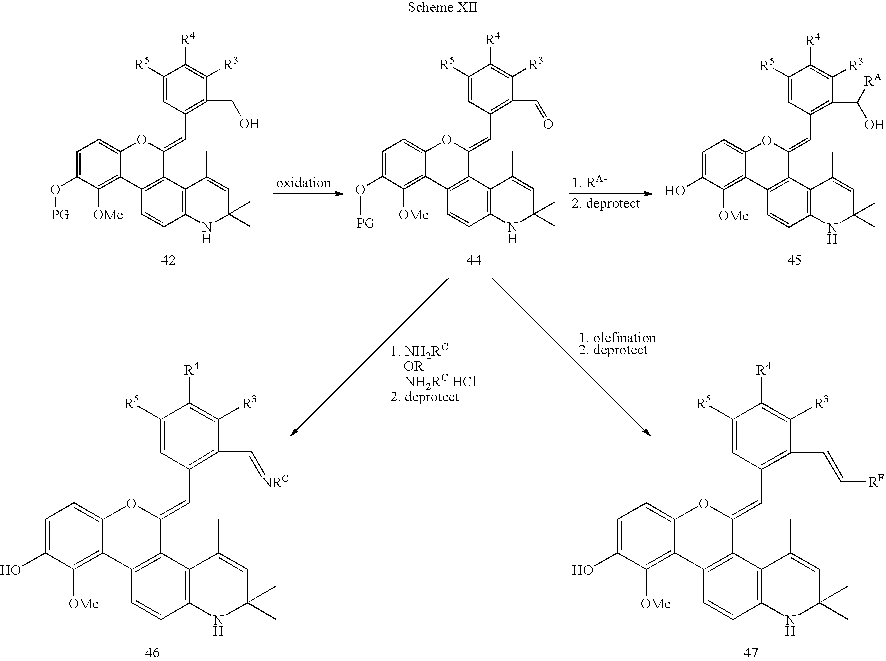

A processes to form compounds of Structure 45, 46 and 47 are depicted in Scheme XII. Structure 42 is treated with an oxidizing agent, for example, 1-hydroxy 1,2-benziodoxal-3(1H)-one-1-oxide (IBX), to afford a compound of Structure 44. Treatment of 44 with a carbon nucleophile, for example, trifluoromethyl anion, generated by treatment of (trifluoromethyl)trimethylsilane with TBAF, affords the corresponding carbonyl adduct. Subsequent deprotection under the appropriate conditions affords a compound of Structure 45. Compounds of Structure 46 can be prepared by deprotection of the phenol protecting group in Structure 44 with an appropriate reagent. For example, when the TIPS protecting group is used, TBAF can be used followed by addition of an amino derivative, for example, hydroxylamine hydrochloride, to afford a compound of Structure 46. Alternatively, the preparation of Structure 46 can proceed from Structure 44 by addition of an amino-derivative, for example, methoxyamine hydrochloride, followed by subsequent deprotection as described above, to afford a compound of Structure 46. Compounds of Structure 47 can be prepared by treatment of Structure 44 with an olefination reagent, for example the Tebbe reagent, followed by deprotection of the phenol protecting group to afford a compound of Structure 47.

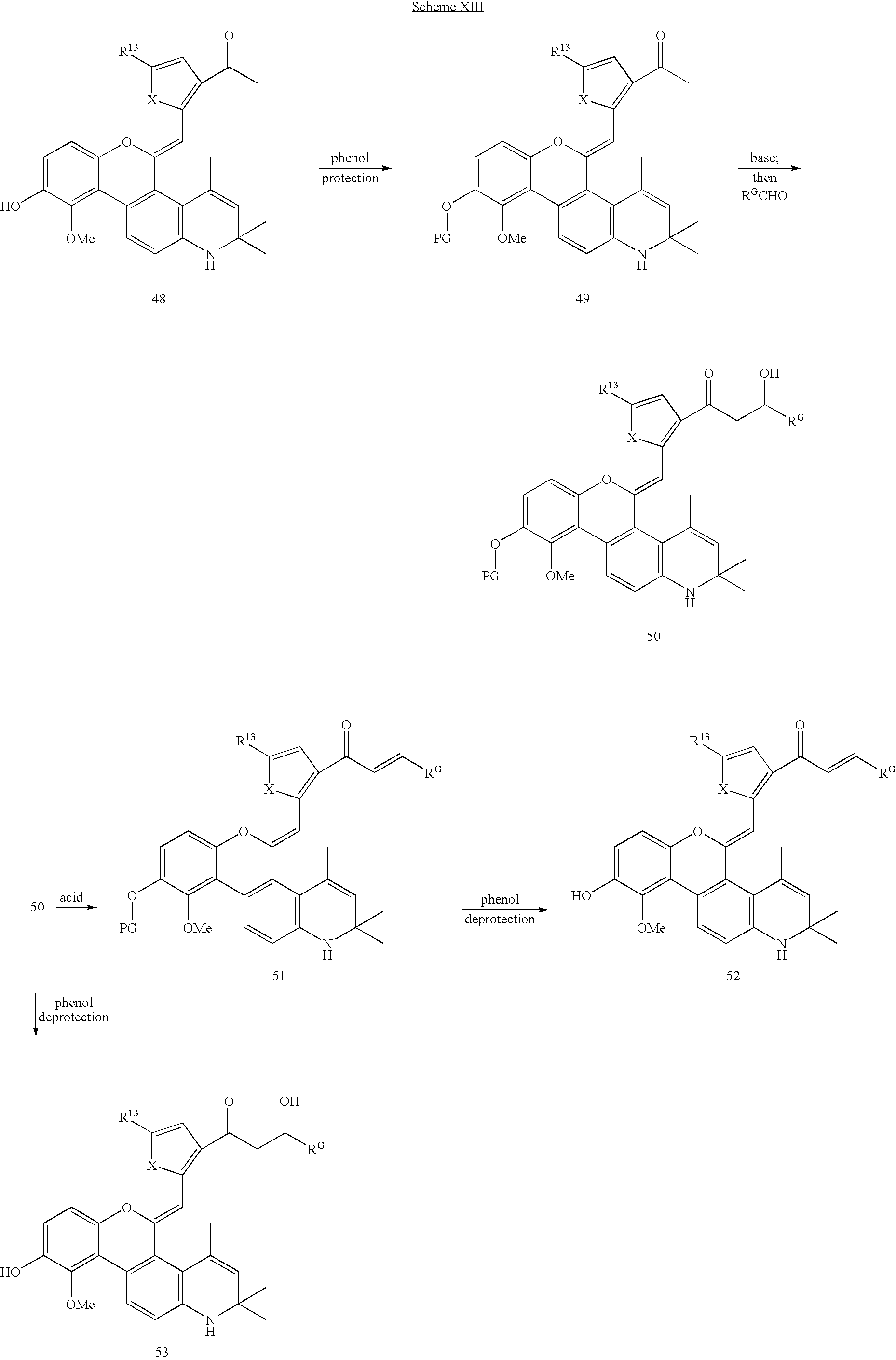

A processes to form compounds of Structure 52 and 53 is depicted in Scheme XIII. Structure 48 is protected at the phenol with a protecting group, for example, triisopropylsilyl triflate, to afford a compound of Structure 49. Structure 49 is then treated with a base, for example, lithium diisopropylamide, and a carbonyl group, for example acetaldehyde, to afford the aldol product of Structure 50. Structure 50 is treated with an acid, for example, p-toluenesulfonic acid, to afford a Structure of compound 51. Deprotection of the phenol with, for example, tetrabutylammonium fluoride, affords a compound of Structure 52. Alternatively, a compound of Structure 50 can be deprotected with, for example, tetrabutylammonium fluoride, to afford a compound of Structure 53. In certain cases, Structure 49 can be transformed directly to compound 51 without isolation of Structure 50.

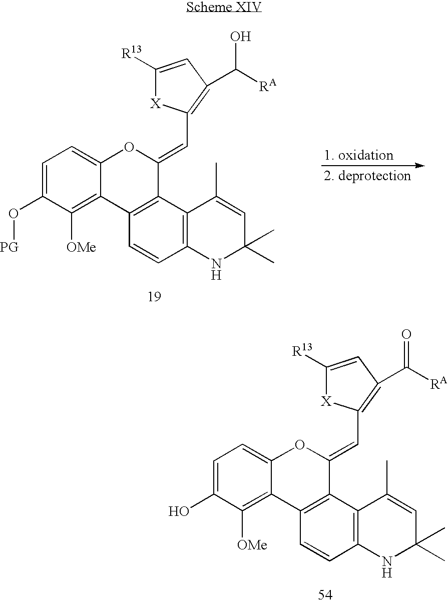

The process to form compounds of Structure 54 is depicted in Scheme XIV. Structure 19 is treated with an oxidizing agent, for example, 1-hydroxy 1,2-benziodoxal-3(1H)-one-1-oxide (IBX), to afford the corresponding carbonyl compound. The phenol protecting group is removed using a suitable set of reaction conditions. For example, when the TIPS protecting group is used (PG=triisopropylsilyl), a fluoride source, for example, TBAF, can be used to afford a compound of Structure 54.

In certain embodiments, provided herein are salts corresponding to any of the compounds provided herein. In certain embodiments, salts corresponding to a selective glucocorticoid receptor modulator or selective glucocorticoid binding agent are provided. In certain embodiments, a salt is obtained by reacting a compound with an inorganic acid, such as hydrochloric acid, hydrobromic acid, sulfuric acid, nitric acid, phosphoric acid, methanesulfonic acid, ethanesulfonic acid, p-toluenesulfonic acid, salicylic acid, and the like. In certain embodiments, a salt is obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like.

In certain embodiments, one or more carbon atoms of a compound provided herein is replaced with silicon. See e.g., WO 03/037905A1; Tacke and Zilch, Endeavour, New Series, 10, 191-197 (1986); Bains and Tacke, Curr. Opin. Drug Discov Devel. Jul:6(4):526-43 (2003). In certain embodiments, compounds provided herein containing one or more silicon atoms possess certain desired properties, including, but not limited to, greater stability and/or longer half-life in a patient, when compared to the same compound in which none of the carbon atoms have been replaced with a silicon atom.

D. Formulation of Pharmaceutical Compositions

The pharmaceutical compositions provided herein contain therapeutically effective amounts of one or more of the glucocorticoid receptor activity modulators provided herein that are useful in the prevention, treatment, or amelioration of one or more of the symptoms of diseases or disorders associated with glucocorticoid receptor activity. Such diseases or disorders include, but are not limited to, inflammation (including, but not limited to, rheumatoid arthritis, asthma (acute and/or chronic), lupus, osteoarthritis, rhinosinusitis, inflammatory bowel disease, polyarteritis nodosa, Wegener's granulomatosis, giant cell arteritis, allergic rhinitis, urticaria, hereditary angioedema, chronic obstructive pulmonary disease, tendonitis, bursitis, autoimmune chronic active hepatitis, cirrhosis), transplant rejection, psoriasis, dermatitus, autoimmune disorders, malignancies (e.g., leukemia, myelomas, lymphomas), acute adrenal insufficiency, congenital adrenal hyperplasia, rheumatic fever, granulomatous disease, immune proliferation/apotosis, HPA axis suppression and regulation, hypercortisolemia, modulation of the Th1/Th2 cytokine balance, chronic kidney disease, stroke and spinal cord injury, hypercalcemia, hyperglycemia, cerebral edema, thrombocytopenia, Little's syndrome, Addison's disease, cystic fibrosis, myasthenia gravis, autoimmune hemolytic anemia, uveitis, pemphigus vulgaris, multiple sclerosis, nasal polyps, sepsis, infections (e.g., bacterial, viral, rickettsial, parasitic), type II diabetes, obesity, metabolic syndrome, depression, schizophrenia, mood disorders, Cushing's syndrome, anxiety, sleep disorders, memory and learning enhancement, or glucocorticoid-induced glaucoma.

The compositions contain one or more compounds provided herein. The compounds are formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. Typically the compounds described above are formulated into pharmaceutical compositions using techniques and procedures well known in the art (see, e.g., Ansel Introduction to Pharmaceutical Dosage Forms, Fourth Edition 1985, 126).

In certain embodiments, a pharmaceutical composition containing one or more compounds provided herein is prepared using known techniques, including, but not limited to mixing, dissolving, granulating, dragee-making, levigating, emulsifying, encapsulating, entrapping or tabletting processes.

In the compositions, effective concentrations of one or more compounds or pharmaceutically acceptable derivatives is (are) mixed with a suitable pharmaceutical carrier or vehicle. The compounds can be derivatized as the corresponding salts, esters, enol ethers or esters, acids, bases, solvates, hydrates or prodrugs prior to formulation, as described above. The concentrations of the compounds in the compositions are effective for delivery of an amount, upon administration, that treats, prevents, or ameliorates one or more of the symptoms of diseases or disorders associated with cytokine activity or in which cytokine activity is implicated. Such diseases or disorders include, but are not limited to, inflammation (including, but not limited to, rheumatoid arthritis, asthma (acute and/or chronic), lupus, osteoarthritis, rhinosinusitis, inflammatory bowel disease, polyarteritis nodosa, Wegener's granulomatosis, giant cell arteritis, allergic rhinitis, urticaria, hereditary angioedema, chronic obstructive pulmonary disease, tendonitis, bursitis, autoimmune chronic active hepatitis, cirrhosis), transplant rejection, psoriasis, dermatitus, autoimmune disorders, malignancies (e.g., leukemia, myelomas, lymphomas), acute adrenal insufficiency, congenital adrenal hyperplasia, rheumatic fever, granulomatous disease, immune proliferation/apotosis, HPA axis suppression and regulation, hypercortisolemia, modulation of the Th1/Th2 cytokine balance, chronic kidney disease, stroke and spinal cord injury, hypercalcemia, hyperglycemia, cerebral edema, thrombocytopenia, Little's syndrome, Addison's disease, cystic fibrosis, myasthenia gravis, autoimmune hemolytic anemia, uveitis, pemphigus vulgaris, multiple sclerosis, nasal polyps, sepsis, infections (e.g., bacterial, viral, rickettsial, parasitic), type II diabetes, obesity, metabolic syndrome, depression, schizophrenia, mood disorders, Cushing's syndrome, anxiety, sleep disorders, memory and learning enhancement, or glucocorticoid-induced glaucoma.

Typically, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds can be formulated as the sole pharmaceutically active ingredient in the composition or can be combined with other active ingredients. Liposomal suspensions, including tissue-targeted liposomes, such as tumor-targeted liposomes, can also be suitable as pharmaceutically acceptable carriers. These can be prepared according to methods known to those skilled in the art. For example, liposome formulations can be prepared as described in U.S. Pat. No. 4,522,811. Briefly, liposomes such as multilamellar vesicles (MLV's) can be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The active compound is included in the pharmaceutically acceptable carrier in an amount sufficient to exert a therapeutically useful effect in the absence of undesirable side effects on the patient treated.

The concentration of active compound in the pharmaceutical composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of diseases or disorders associated with cytokine activity or in which cytokine activity is implicated, as described herein.

The effective amount of a compound provided herein can be determined by one of ordinary skill in the art, and includes exemplary dosage amounts for a mammal of from about 0.05 to 100 mg/kg of body weight of active compound per day, which can be administered in a single dose or in the form of individual divided doses, such as from 1 to 4 times per day. It will be understood that the specific dose level and frequency of dosage for any particular subject can be varied and wilt depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

The active ingredient can be administered at once, or can be divided into a number of smaller doses to be administered at intervals of time. It is understood that the precise dosage and duration of treatment is a function of the disease being treated and can be determined empirically using known testing protocols or by extrapolation from in vivo or in vitro test data. It is to be noted that concentrations and dosage values can also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the compounds, compositions, methods and other subject matter provided herein.

Pharmaceutically acceptable derivatives include acids, bases, enol ethers and esters, salts, esters, hydrates, solvates and prodrug forms. The derivative is selected such that its pharmacokinetic properties are superior to the corresponding neutral compound.

Thus, effective concentrations or amounts of one or more of the compounds described herein or pharmaceutically acceptable derivatives thereof are mixed with a suitable pharmaceutical carrier or vehicle for systemic, topical or local administration to form pharmaceutical compositions. Compounds are included in an amount effective for ameliorating one or more symptoms of, or for treating or preventing diseases or disorders associated with glucocorticoid receptor activity or in which glucocorticoid receptor activity is implicated, as described herein. The concentration of active compound in the composition will depend on absorption, inactivation, excretion rates of the active compound, the dosage schedule, amount administered, particular formulation as well as other factors known to those of skill in the art.

The compositions are intended to be administered by a suitable route, including orally in form of capsules, tablets, granules, powders or liquid formulations including syrups; parenterally, such as subcutaneously, intravenously, intramuscularly, with inteasternal injection or infusion techniques (as sterile injectable aq. or non-aq. solutions or suspensions); nasally such as by inhalation spray; topically, such as in the form of a cream or ointment; rectally such as in the form of suppositories; liposomally; and locally. The compositions can be in liquid, semi-liquid or solid form and are formulated in a manner suitable for each route of administration. In certain embodiments, administration of the formulation include parenteral and oral modes of administration. In one embodiment, the compositions are administered orally.

In certain embodiments, the pharmaceutical compositions provided herein containing one or more compounds provided herein is a solid (e.g., a powder, tablet, and/or capsule). In certain of such embodiments, a solid of the pharmaceutical composition containing one or more compounds provided herein is prepared using ingredients known in the art, including, but not limited to, starches, sugars, diluents, granulating agents, lubricants, binders, and disintegrating agents.

In certain embodiments, a pharmaceutical composition containing one or more compounds provided herein is formulated as a depot preparation. Certain of such depot preparations are typically longer acting than non-depot preparations. In certain embodiments, such preparations are administered by implantation (for example subcutaneously or intramuscularly) or by intramuscular injection. In certain embodiments, depot preparations are prepared using suitable polymeric or hydrophobic materials (for example an emulsion in an acceptable oil) or ion exchange resins, or as sparingly soluble derivatives, for example, as a sparingly soluble salt.

In certain embodiments, a pharmaceutical composition containing one or more compounds provided herein contains a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, a pharmaceutical composition containing one or more compounds provided herein contains one or more tissue-specific delivery molecules designed to deliver the pharmaceutical composition to specific tissues or cell types. For example, in certain embodiments, pharmaceutical compositions include liposomes coated with a tissue-specific antibody.

In certain embodiments, a pharmaceutical composition containing one or more compounds provided herein contains a co-solvent system. Certain of such co-solvent systems contain, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co solvent system, which is a solution of absolute ethanol comprising 3% w/v benzyl alcohol, 8% w/v of the nonpolar surfactant Polysorbate 80™, and 65% w/v polyethylene glycol 300. The proportions of such co solvent systems may be varied considerably without significantly altering their solubility and toxicity characteristics. Furthermore, the identity of co solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, solutions or suspensions used for parenteral, intradermal, subcutaneous, or topical application can include any of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, disposable syringes or single or multiple dose vials made of glass, plastic or other suitable material.

In instances in which the compounds exhibit insufficient solubility, methods for solubilizing compounds can be used. Such methods are known to those of skill in this art, and include, but are not limited to, using cosolvents, such as dimethylsulfoxide (DMSO), using surfactants, such as TWEEN®, or dissolution in aqueous sodium bicarbonate. Derivatives of the compounds, such as prodrugs of the compounds can also be used in formulating effective pharmaceutical compositions.

In certain embodiments, a pharmaceutical composition containing one or more compounds provided herein contains a sustained release system. A non-limiting example of such a sustained-release system is a semipermeable matrix of solid hydrophobic polymers. In certain embodiments, sustained release systems may, depending on their chemical nature, release compounds over a period of hours, days, weeks or months.

In certain embodiments, upon mixing or addition of the compound(s), the resulting mixture can be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and can be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically active compounds and derivatives thereof are typically formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms can be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

The composition can contain along with the active ingredient: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polyvinylpyrrolidine, celluloses and derivatives thereof, povidone, crospovidones and other such binders known to those of skill in the art. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered can also contain minor amounts of nontoxic auxiliary substances such as wetting agents, emulsifying agents, or solubilizing agents, pH buffering agents and the like, for example, acetate, sodium citrate, cyclodextrin derivatives, sorbitan monolaurate, triethanolamine sodium acetate, triethanolamine oleate, and other such agents. Actual methods of preparing such dosage forms are known, or will be apparent, to those skilled in this art; for example, see Remington's Pharmaceutical Sciences, Mack Publishing Company, Easton, Pa., 15th Edition, 1975. The composition or formulation to be administered will, in any event, contain a quantity of the active compound in an amount sufficient to alleviate the symptoms of the treated subject.

Dosage forms or compositions containing active ingredient in the range of 0.005% to 100% with the balance made up from non-toxic carrier can be prepared. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions can contain 0.001%-100% active ingredient, in one embodiment 0.1-85%, in another embodiment 75-95%.

In certain embodiments, the compounds can be administered in a form suitable for immediate release or extended release. Immediate release or extended release can be achieved with suitable pharmaceutical compositions or, particularly in the case of extended release, with devices such as subcutaneous implants or osmotic pumps. Exemplary compositions for topical administration include a topical carrier such as PLASTIBASE® (mineral oil gelled with polyethylene).

In certain embodiments, compounds used in the pharmaceutical compositions may be provided as pharmaceutically acceptable salts with pharmaceutically compatible counterions. Pharmaceutically compatible salts may be formed with many acids, including but not limited to hydrochloric, sulfuric, acetic, lactic, tartaric, malic, succinic, etc.

In certain embodiments, the pharmaceutical compositions contain a compound provided herein in a therapeutically effective amount. In certain embodiments, the therapeutically effective amount is sufficient to prevent, alleviate or ameliorate symptoms of a disease or to prolong the survival of the subject being treated. Determination of a therapeutically effective amount is well within the capability of those skilled in the art.

The compositions can include other active compounds to obtain desired combinations of properties. The compounds provided herein, or pharmaceutically acceptable derivatives thereof as described herein, can also be advantageously administered for therapeutic or prophylactic purposes together with another pharmacological agent known in the general art to be of value in treating one or more of the diseases or medical conditions referred to hereinabove, such as diseases or disorders associated with nuclear receptor activity or in which nuclear receptor activity is implicated. It is to be understood that such combination therapy constitutes a further aspect of the compositions and methods of treatment provided herein.

In certain embodiments, a pharmaceutical composition containing one or more compounds provided herein is formulated as a prodrug. In certain embodiments, prodrugs are useful because they are easier to administer than the corresponding active form. For example, in certain instances, a prodrug may be more bioavailable (e.g., through oral administration) than is the corresponding active form. In certain instances, a prodrug may have improved solubility compared to the corresponding active form. In certain embodiments, a prodrug is an ester. In certain embodiments, such prodrugs are less water soluble than the corresponding active form. In certain instances, such prodrugs possess superior transmittal across cell membranes, where water solubility is detrimental to mobility. In certain embodiments, the ester in such prodrugs is metabolically hydrolyzed to carboxylic acid. In certain instances the carboxylic acid containing compound is the corresponding active form. In certain embodiments, a prodrug comprises a short peptide polyaminoacid) bound to an acid group. In certain of such embodiments, the peptide is metabolized to form the corresponding active form.

In certain embodiments, a pharmaceutical composition containing one or more compounds provided herein is useful for treating a conditions or disorder in a mammalian, and particularly in a human patient. Suitable administration routes include, but are not limited to, oral, rectal, transmucosal, intestinal, enteral, topical, suppository, through inhalation, intrathecal, intraventricular, intraperitoneal, intranasal, intraocular and parenteral (e.g., intravenous, intramuscular, intramedullary, and subcutaneous). In certain embodiments, pharmaceutical compositions are administered to achieve local rather than systemic exposures. For example, pharmaceutical compositions may be injected directly in the area of desired effect (e.g., in the renal or cardiac area). In certain embodiments in which the pharmaceutical composition is administered locally, the dosage regimen is adjusted to achieve a desired local concentration of a compound provided herein.

In certain embodiments, a pharmaceutical composition containing one or more compounds provided herein is administered in the form of a dosage unit (e.g., tablet, capsule, bolus, etc.). In certain embodiments, such dosage units comprise a selective glucocorticoid receptor modulator in a dose from about 1 μg/kg of body weight to about 50 mg/kg of body weight. In certain embodiments, such dosage units comprise a selective glucocorticoid receptor modulator in a dose from about 2 μg/kg of body weight to about 25 mg/kg of body weight. In certain embodiments, such dosage units comprise a selective glucocorticoid receptor modulator in a dose from about 10 μg/kg of body weight to about 5 mg/kg of body weight. In certain embodiments, pharmaceutical compositions are administered as needed, once per day, twice per day, three times per day, or four or more times per day. It is recognized by those skilled in the art that the particular dose, frequency, and duration of administration depends on a number of factors, including, without limitation, the biological activity desired, the condition of the patient, and tolerance for the pharmaceutical composition.

In certain embodiments, a pharmaceutical composition provided herein is administered for a period of continuous therapy. For example, a pharmaceutical composition provided herein may be administered over a period of days, weeks, months, or years.

Dosage amount, interval between doses, and duration of treatment may be adjusted to achieve a desired effect. In certain embodiments, dosage amount and interval between doses are adjusted to maintain a desired concentration of compound in a patient. For example, in certain embodiments, dosage amount and interval between doses are adjusted to provide plasma concentration of a compound provided herein at an amount sufficient to achieve a desired effect. In certain of such embodiments the plasma concentration is maintained above the minimal effective concentration ALEC). In certain embodiments, pharmaceutical compositions provided herein are administered with a dosage regimen designed to maintain a concentration above the MEC for 10-90% of the time, between 30-90% of the time, or between 50-90% of the time.

In certain embodiments, a pharmaceutical composition containing a compound provided herein is prepared for oral administration. In certain of such embodiments, a pharmaceutical composition is formulated by combining one or more compounds provided herein with one or more pharmaceutically acceptable carriers. Certain of such carriers enable compounds provided herein to be formulated as tablets, pills, dragees, capsules, liquids, gels, syrups, slurries, suspensions and the like, for oral ingestion by a patient. In certain embodiments, pharmaceutical compositions for oral use are obtained by mixing one or more compounds provided herein and one or more solid excipient. Suitable excipients include, but are not limited to, fillers, such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). In certain embodiments, such a mixture is optionally ground and auxiliaries are optionally added. In certain embodiments, pharmaceutical compositions are formed to obtain tablets or dragee cores. In certain embodiments, disintegrating agents (e.g., cross linked polyvinyl pyrrolidone, agar, or alginic acid or a salt thereof, such as sodium alginate) are added.

In certain embodiments, dragee cores are provided with coatings. In certain of such embodiments, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to tablets or dragee coatings.

1. Compositions for Oral Administration

In certain embodiments, oral pharmaceutical dosage forms are either solid, gel or liquid. The solid dosage forms are tablets, capsules, granules, and bulk powders. Types of oral tablets include compressed, chewable lozenges and tablets which can be enteric-coated, sugar-coated or film-coated. Capsules can be hard or soft gelatin capsules, while granules and powders can be provided in non-effervescent or effervescent form with the combination of other ingredients known to those skilled in the art.

In certain embodiments, the formulations are solid dosage forms, preferably capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

In certain embodiments, pharmaceutical compositions for oral administration are push fit capsules made of gelatin. Certain of such push fit capsules comprise one or more compounds provided herein in admixture with one or more filler such as lactose, binders such as starches, and/or lubricants such as talc or magnesium stearate and, optionally, stabilizers. In certain embodiments, pharmaceutical compositions for oral administration are soft, sealed capsules made of gelatin and a plasticizer, such as glycerol or sorbitol. In certain soft capsules, one or more compounds provided are to be dissolved or suspended in suitable liquids, such as fatty oils, liquid paraffin, or liquid polyethylene glycols. In addition, stabilizers may be added.

In certain embodiments, pharmaceutical compositions are prepared for buccal administration. Certain of such pharmaceutical compositions are tablets or lozenges formulated in conventional manner.

Examples of binders for use in the compositions provided herein include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, sodium alginate, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the compound could be provided in a composition that protects it from the acidic environment of the stomach. For example, the composition can be formulated in an enteric coating that maintains its integrity in the stomach and releases the active compound in the intestine. The composition can also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup can contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient can be included.

Pharmaceutically acceptable carriers included in tablets are binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets are compressed tablets to which different layers of pharmaceutically acceptable substances are applied. Film-coated tablets are compressed tablets which have been coated with a polymer or other suitable coating. Multiple compressed tablets are compressed tablets made by more than one compression cycle utilizing the pharmaceutically acceptable substances previously mentioned. Coloring agents can also be used in the above dosage forms. Flavoring and sweetening agents are used in compressed tablets, sugar-coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and can contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives, Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such Suits, and synthetic blends of compounds which produce a pleasant taste sensation.

For a solid dosage form, the solution or suspension, in for example propylene carbonate, vegetable oils or triglycerides, is preferably encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, can be diluted with a sufficient quantity of a pharmaceutically acceptable liquid carrier, e.g., water, to be easily measured for administration.

Alternatively, liquid or semi-solid oral formulations can be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. Re 28,819 and 4,358,603. Briefly, such formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl)acetals of lower alkyl aldehydes such as acetaldehyde diethyl acetal.

In all embodiments, tablets and capsules formulations can be coated as known by those of skill in the art in order to modify or sustain dissolution of the active ingredient. Thus, for example, they can be coated with a conventional enterically digestible coating, such as phenylsalicylate, waxes and cellulose acetate phthalate.

Exemplary compositions can include fast-dissolving diluents such as mannitol, lactose, sucrose, and/or cyclodextrins. Also included in such formulations can be high molecular weight excipients such as celluloses (AVICEL®) or polyethylene glycols (PEG); an excipient to aid mucosal adhesion such as hydroxypropyl cellulose (HPC), hydroxypropyl methyl cellulose (HPMC), sodium carboxymethyl cellulose (SCMC), and/or maleic anhydride copolymer (e.g., GANTREZ®); and agents to control release such as polyacrylic copolymer (e.g., CARBOPOL 934®). Lubricants, glidants, flavors, coloring agents and stabilizers can also be added for ease of fabrication and use.

In certain embodiments, a daily dosage regimen for a patient contains an oral dose of between 0.1 mg and 2000 mg of a compound provided herein. In certain embodiments, a daily dosage regimen is administered as a single daily dose. In certain embodiments, a daily dosage regimen is administered as two, three, four, or more than four doses.

2. Injectables, Solutions and Emulsions

In certain embodiments, the pharmaceutical composition is prepared for transmucosal administration. In certain of such embodiments penetrants appropriate to the barrier to be permeated are used in the formulation. Such penetrants are generally known in the art.

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol, mannitol, 1,3-butanediol, Ringer's solution, an isotonic sodium chloride solution or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, mono- or diglycerides, fatty acids, such as oleic acid, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylenevinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. Typically a therapeutically effective dosage is formulated to contain a concentration of at least about 0.1% w/w up to about 90% w/w or more, preferably more than 1% w/w of the active compound to the treated tissue(s). The active ingredient can be administered at once, or can be divided into a number of smaller doses to be administered at intervals of time. It is understood that the precise dosage and duration of treatment is a function of the tissue being treated and can be determined empirically using known testing protocols or by extrapolation from in vivo or in vitro test data. It is to be noted that concentrations and dosage values can also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of formulations provided herein.

The compounds can be formulated in any suitable vehicle or form. For example, they can be in micronized or other suitable form and/or can be derivatized to produce a more soluble active product or to produce a prodrug or for other purposes. The form of the resulting mixture depends upon a number of factors, including, for example, an intended mode of administration and the solubility of the compound in the selected carrier or vehicle. The effective concentration is sufficient for ameliorating the symptoms of the condition and can be empirically determined.

In certain embodiments, a pharmaceutical composition is prepared for administration by injection wherein the pharmaceutical composition contains a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampules or in multi dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the compounds to allow for the preparation of highly concentrated solutions.

In certain embodiments, the pharmaceutical composition is prepared for administration by inhalation. Certain of such pharmaceutical compositions for inhalation are prepared in the form of an aerosol spray in a pressurized pack or a nebulizer. Certain of such pharmaceutical compositions contain a propellant, e.g., dichlorodifluoromethane, trichlorofluoromethane, dichlorotetrafluoroethane, carbon dioxide or other suitable gas. In certain embodiments using a pressurized aerosol, the dosage unit may be determined with a valve that delivers a metered amount. In certain embodiments, capsules and cartridges for use in an inhaler or insufflator may be formulated. Certain of such formulations contain a powder mixture of a compound provided herein and a suitable powder base such as lactose or starch.

In certain embodiments, the pharmaceutical compositions provided are administered by continuous intravenous infusion. In certain of such embodiments, from 0.1 mg to 500 mg of the composition is administered per day.

3. Lyophilized Powders

Of interest herein are also lyophilized powders, which can be reconstituted for administration as solutions, emulsions and other mixtures. They can also be reconstituted and formulated as solids or gels.

The sterile, lyophilized powder is prepared by dissolving a compound provided herein, or a pharmaceutically acceptable derivative thereof, in a suitable solvent. The solvent can contain an excipient which improves the stability or other pharmacological component of the powder or reconstituted solution, prepared from the powder. Excipients that can be used include, but are not limited to, dextrose, sorbital, fructose, corn syrup, xylitol, glycerin, glucose, sucrose or other suitable agent. The solvent can also contain a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage 10-1000 mg, in one embodiment, 100-500 mg or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, preferably 5-35 mg, more preferably about 9-30 mg of lyophilized powder, is added per mL of sterile water or other suitable carrier. The precise amount depends upon the selected compound. Such amount can be empirically determined.

4. Topical Administration

Topical mixtures are prepared as described for the local and systemic administration. The resulting mixture can be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof can be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will typically have diameters of less than 50 microns, preferably less than 10 microns.

In certain embodiments, the pharmaceutical compositions for inhalation are prepared in the form of an aerosol spray in a pressurized pack or a nebulizer. Certain of such pharmaceutical compositions contain a propellant, e.g., dichlorodifluoromethane, trichlorofluoromethane, dichlorotetrafluoroethane, carbon dioxide or other suitable gas. In certain embodiments using a pressurized aerosol, the dosage unit can be determined with a valve that delivers a metered amount. In certain embodiments, capsules and cartridges for use in an inhaler or insufflator can be formulated. Certain of such formulations contain a powder mixture of a compound provided herein and a suitable powder base such as lactose or starch.

Exemplary compositions for nasal aerosol or inhalation administration include solutions which can contain, for example, benzyl alcohol or other suitable preservatives, absorption promoters to enhance absorption and/or bioavailability, and/or other solubilizing or dispersing agents such as those known in the art.

The compounds can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered. These solutions, particularly those intended for ophthalmic use, can be formulated as 0.01%-10% isotonic solutions, pH about 5-7, with appropriate salts.

In certain embodiments, the pharmaceutical composition is prepared for topical administration. Certain of such pharmaceutical compositions contain bland moisturizing bases, such as ointments or creams. Exemplary suitable ointment bases include, but are not limited to, petrolatum, petrolatum plus volatile silicones, lanolin and water in oil emulsions such as Eucerin™, available from Beiersdorf (Cincinnati, Ohio). Exemplary suitable cream bases include, but are not limited to, Nivea™ Cream, available from Beiersdorf (Cincinnati, Ohio), cold cream (USP), Purpose Cream™, available from Johnson & Johnson (New Brunswick, N.J.), hydrophilic ointment (USP) and Lubriderm™, available from Pfizer (Morris Plains, N.J.).

In certain embodiments, the formulation, route of administration and dosage for the pharmaceutical composition provided herein can be chosen in view of a particular patient's condition. (See e.g., Fingl et al. 1975, in “The Pharmacological Basis of Therapeutics”, Ch. 1 p. 1). In certain embodiments, the pharmaceutical composition is administered as a single dose. In certain embodiments, a pharmaceutical composition is administered as a series of two or more doses administered over one or more days.

5. Compositions for Other Routes of Administration

Other routes of administration, such as topical application, transdermal patches, and rectal administration are also contemplated herein.

In certain embodiments, the pharmaceutical composition is prepared for topical administration such as rectal administration. The pharmaceutical dosage forms for rectal administration include, but are not limited to rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases can be used. In certain embodiments, the pharmaceutical compositions contain bland moisturizing bases, such as ointments or creams. Exemplary suitable ointment bases include, but are not limited to, petrolatum, petrolatum plus volatile silicones, lanolin and water in oil emulsions such as Eucerin™, available from Beiersdorf (Cincinnati, Ohio). Exemplary suitable cream bases include, but are not limited to, Nivea™ Cream, available from Beiersdorf (Cincinnati, Ohio), cold cream (USP), Purpose Cream™, available from Johnson & Johnson (New Brunswick, N.J.), hydrophilic ointment (USP) and Lubriderm™, available from Pfizer (Morris Plains, N.J.). Agents to raise the melting point of suppositories include spermaceti and wax. Rectal suppositories can be prepared either by the compressed method or by molding. The typical weight of a rectal suppository is about 2 to 3 gm.

Tablets and capsules for rectal administration are manufactured using the same pharmaceutically acceptable substance and by the same methods as for formulations for oral administration.

6. Articles of Manufacture

The compounds or pharmaceutically acceptable derivatives can be packaged as articles of manufacture containing packaging material, within the packaging material a compound or pharmaceutically acceptable derivative thereof provided herein, which is effective for modulating the activity of glucocorticoid receptor, or for treatment, prevention or amelioration of one or more symptoms of glucocorticoid receptor mediated diseases or disorders, or diseases or disorders in which glucocorticoid receptor activity is implicated, and a label that indicates that the compound or composition, or pharmaceutically acceptable derivative thereof, is used for modulating the activity of glucocorticoid receptor or for treatment, prevention or amelioration of one or more symptoms of glucocorticoid receptor mediated diseases or disorders, or diseases or disorders in which glucocorticoid receptor activity is implicated.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease or disorder in which glucocorticoid receptor activity is implicated as a mediator or contributor to the symptoms or cause.

In certain embodiments, the pharmaceutical compositions can be presented in a pack or dispenser device which can contain one or more unit dosage forms containing a compound provided herein. The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions containing a compound provided herein formulated in a compatible pharmaceutical carrier can also be prepared, placed in an appropriate container, and labeled for treatment of an indicated condition.

E. Evaluation of the Activity of the Compounds

Standard physiological, pharmacological and biochemical procedures are available for testing the compounds provided herein to identify those that possess activity as glucocorticoid receptor modulators. In vitro and in vivo assays known in the art can be used to evaluate the activity of the compounds provided herein as glucocorticoid receptor modulators. Exemplary assays include, but are not limited to fluorescence polarization assay, luciferase assay, co-transfaction assay. In certain embodiments, the compounds provided herein are capable of modulating activity of glucocorticoid receptor in a “co-transfection” assay (also called a “cis-trans” assay), which is known in the art. See e.g., Evans et al., Science, 240:889-95 (1988); U.S. Pat. Nos. 4,981,784 and 5,071,773; Pathirana et al, “Nonsteroidal Human Progesterone Receptor Modulators from the Marie Alga Cymopolia Barbata,” Mol. Pharm. 47:630-35 (1995)). Modulating activity in a co-transfection assay has been shown to correlate with in vivo modulating activity. Thus, in certain embodiments, such assays are predictive of in vivo activity. See, e.g, Berger et al., J. Steroid Biochem. Molec. Biol. 41:773 (1992).

In certain co-transfection assays, two different co-transfection plasmids are prepared. In the first co-transfection plasmid, cloned cDNA encoding an intracellular receptor (e.g., glucocorticoid receptor) is operatively linked to a constitutive promoter (e.g., the SV 40 promoter). In the second co-transfection plasmid, cDNA encoding a reporter protein, such as firefly luciferase (LUC), is operatively linked to a promoter that is activated by a receptor-dependant activation factor. Both co-transfection plasmids are co-transfected into the same cells. Expression of the first co-transfection plasmid results in production of the intracellular receptor protein. Activation of that intracellular receptor protein (e.g., by binding of an agonist) results in production of a receptor-dependant activation factor for the promoter of the second co-transfection plasmid. That receptor-dependant activation factor in turn results in expression of the reporter protein encoded on the second co-transfection plasmid. Thus, reporter protein expression is linked to activation of the receptor. Typically, that reporter activity can be conveniently measured (e.g., as increased luciferase production).

Certain co-transfection assays can be used to identify agonists, partial agonists, and/or antagonists of intracellular receptors. In certain embodiments, to identify agonists, co-transfected cells are exposed to a test compound. If the test compound is an agonist or partial agonist, reporter activity is expected to increase compared to co-transfected cells in the absence of the test compound. In certain embodiments, to identify antagonists, the cells are exposed to a known agonist (e.g., glucocorticoid for the glucocorticoid receptor) in the presence and absence of a test compound. If the test compound is an antagonist, reporter activity is expected to decrease relative to that of cells exposed only to the known agonist.

In certain embodiments, the compounds provided are used to detect the presence, quantity and/or state of receptors in a sample. In certain of such embodiments, samples are obtained from a patient. In certain embodiments, compounds are radio- or isotopically-labeled. For example, the compounds provided herein that selectively bind glucocorticoid receptor can be used to determine the presence of such receptors in a sample, such as cell homogenates and lysates.

F. Methods of Use of the Compounds and Compositions

Methods of use of the compounds and compositions provided herein also are provided. The methods include in vitro and in vivo uses of the compounds and compositions for altering glucocorticoid receptor activity and for treatment, prevention, or amelioration of one or more symptoms of diseases or disorder that are modulated by glucocorticoid receptor activity, or in which glucocorticoid receptor activity, is implicated. In certain embodiments, provided herein are methods of treating a patient by administering a compound provided herein. In certain embodiments, such patient exhibits symptoms or signs of a glucocorticoid receptor mediated condition.

Exemplary conditions that can be treated with compounds provided herein include, but are not limited to, inflammation (including, but not limited to, rheumatoid arthritis, asthma, lupus, osteoarthritis, rhinosinusitis, inflammatory bowel disease, polyarteritis nodosa, Wegener's granulomatosis, giant cell arteritis, allergic rhinitis, urticaria, hereditary angioedema, chronic obstructive pulmonary disease, tendonitis, bursitis, autoimmune chronic active hepatitis, cirrhosis), transplant rejection, psoriasis, dermatitus, autoimmune disorders, malignancies (leukemia, myelomas, lymphomas), acute adrenal insufficiency, congenital adrenal hyperplasia, rheumatic fever, granulomatous disease, immune proliferation/apotosis, HPA axis suppression and regulation, hypercortisolemia, Th1/Th2 cytokine related disorders, chronic kidney disease, stroke and spinal cord injury, hypercalcemia, hyperglycemia, cerebral edema, thrombocytopenia, Little's syndrome, Addison's disease, cystic fibrosis, myasthenia gravis, autoimmune hemolytic anemia, uveitis, pemphigus vulgaris, multiple sclerosis, nasal polyps, sepsis, infections (bacterial, viral, rickettsial, parasitic), type II diabetes, obesity, metabolic syndrome, depression, schizophrenia, mood disorders, Cushing's syndrome, anxiety, sleep disorders, memory and learning enhancement, and glaucoma.

In certain embodiments, the compounds provided are used to treat arthritis. In certain embodiments, the compounds are used to treat asthma, including chronic asthma and/or acute asthma. In certain embodiments, the compounds are used treat multiple sclerosis.

In certain embodiments, the compounds provided are used to treat cancer. Certain exemplary cancers include, but are not limited to, lung cancer, head squamous cancer, neck squamous cancer, colorectal cancer, prostate cancer, breast cancer, acute lymphocytic leukemia, adult acute myeloid leukemia, adult non-Hodgkin's lymphoma, brain tumor, cervical cancer, childhood cancer, childhood sarcoma, chronic lymphocytic leukemia, chronic myeloid leukemia, esophageal cancer, hairy cell leukemia, kidney cancer, liver cancer, multiple myeloma, neuroblastoma, oral cancer, pancreatic cancer, primary central nervous system lymphoma, skin cancer or small-cell lung cancer. In certain embodiments, the cancer is colorectal cancer, gastric carcinoma, glioma, head and neck squamous cell carcinoma, papillary renal carcinoma, leukemia, lymphoma, Li-Fraumeni syndrome, malignant pleural mesothelioma, melanoma, multiple myeloma, non-small cell lung cancer, synovial sarcoma, thyroid carcinoma, and transitional cell carcinoma of urinary bladder.

G. Combination Therapies

In certain embodiments, one or more compounds provided herein are co-administered with one or more other pharmaceutical agents or treatments. In certain embodiments, such one or more other pharmaceutical agents are designed to treat the same disease or condition as the compounds provided herein. In certain embodiments, such one or more other pharmaceutical agents are designed to treat a different disease or condition as the compounds provided herein. In certain embodiments, such one or more other pharmaceutical agents are designed to treat an undesired effect of the compounds provided herein. In certain embodiments, the compounds provided herein is co-administered with another pharmaceutical agent to treat an undesired effect of that other pharmaceutical composition. In certain embodiments, the compounds provided herein and one or more other pharmaceutical agents are administered at the same time. In certain embodiments, the compounds provided herein and one or more other pharmaceutical agents are administered at different times. In certain embodiments, the compounds provided herein and one or more other pharmaceutical agents are prepared together in a single formulation. In certain embodiments, the compounds provided herein and one or more other pharmaceutical agents are prepared separately.

Examples of pharmaceutical agents that can be co-administered with the compounds provided herein include, but are not limited to, analgesics (e.g., acetaminophen); anti-inflammatory agents, including, but not limited to non-steroidal anti-inflammatory drugs (e.g., ibuprofen, COX-1 inhibitors, and COX-2, inhibitors); salicylates; antibiotics; antivirals; antifungal agents; antidiabetic agents (e.g., biguanides, glucosidase inhibitors, insulins, sulfonylureas, and thiazolidenediones); adrenergic modifiers; diuretics; hormones (e.g., anabolic steroids, androgen, estrogen, calcitonin, progestin, somatostan, and thyroid hormones); immunomodulators; muscle relaxants; antihistamines; osteoporosis agents (e.g., biphosphonates, calcitonin, and estrogens); prostaglandins, antineoplastic agents; psychotherapeutic agents; sedatives; poison oak or poison sumac products; antibodies; and vaccines.

The above other pharmaceutical agents, when employed in combination with the compounds provided herein, can be used, for example, in those amounts indicated in the Physicians' Desk Reference (PDR) or as otherwise determined by one of ordinary skill in the art. In the methods provided herein, such other pharmaceutical agent(s) can be administered prior to, simultaneously with, or following the administration of the compounds provided herein.

### EXAMPLES

The following examples, including experiments and results achieved, are provided for illustrative purposes only and are not to be construed as limiting the scope of claimed subject matter.

### Example 1

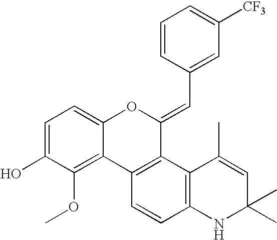

(Z)-5-(3′-trifluoromethylbenzylidene)-1,2-dihydro-9-hydroxy-10-methoxy-2,2,4-trimethyl-5H-chromeno[3,4-f]quinoline (Compound 11, structure 1 of Scheme I, where R¹=3-trifluoromethylphenyl

General Method 1: To a flame-dried 2-neck, 10 mL round bottom flask fitted with a reflux condenser was added magnesium turnings (28 mg, 2.0 mmol) and diethyl ether (3 mL). A solution of 3-trifluoromethylbenzyl bromide (478 mg, 2.0 mmol) in diethyl ether was added to the slurry of magnesium turnings. After 1 h, a solution of 9-hydroxy-10-methoxy-2,2,4-trimethyl-1,2-dihydro-5H-chromeno[3,4-f]quinoline-5-one (Compound A, Scheme 1) (30 mg, 0.09 mmol) in diethyl ether (1 mL) was added. After 18 h, the reaction was quenched with ammonium chloride (3 mL), extracted with ethyl acetate (2×10 mL), washed with brine (2×10 mL), dried over magnesium sulfate, filtered, and concentrated. The crude product was purified by precipitation from dichloromethane/hexanes and collected by filtration. The product was then dissolved in dichloromethane and treated with p-toluenesulfonic acid (catalytic) and followed by TLC (0.1% triethylamine/dichloromethane). After 20 min, the solution was filtered on silica gel, washed with dichloromethane and concentrated. The crude product was then purified by flash chromatography (0.1% triethylamine/dichloromethane) to afford the title compound. ¹H NMR (400 MHz, CDCl₃) δ 8.18 (d, J=8.6 Hz, 1H), 8.10 (s, 1H), 7.91-7.84 (m, 1H), 7.48-7.41 (m, 2-overlapping signals, 2H), 6.87 (d, J=10.8 Hz, 1H), 6.85 (d, J=10.8 Hz, 1H), 6.69 (d, J=8.6 Hz, 1H), 5.65 (s, 1H), 5.56 (s, 1H), 5.53 (s, 1H), 4.21 (br s, 1H), 3.78 (s, 3H), 2.10 (s, 3H), 1.37 (br s, 6H).

### Example 2

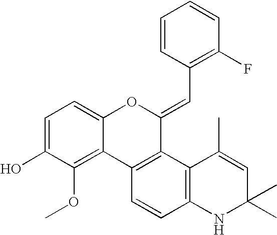

(Z)-5-(2′-fluorobenzylidene)-1,2-dihydro-9-hydroxy-10-methoxy-2,2,4-trimethyl-5H-chromeno[3,4-f]quinoline (Compound 12, structure 1 of Scheme I, where R¹=2-fluorophenyl)

This compound was prepared according to General Method 1 (Example 1) from 2-fluorobenzyl bromide. ¹H NMR (500 MHz, CDCl₃) δ 8.25 (m, 1H), 8.17 (d, J=8.8 Hz, 1H), 7.19 (m, 2H), 7.07 (m, 1H), 6.85 (d, J=8.8 Hz, 1H), 6.69 (d, J=8.3 Hz, 1H), 6.74 (d, J=8.8 Hz, 1H), 5.92 (s, 1H), 5.53 (s, 1H), 3.78 (s, 3H), 2.12 (d, J=1.0 Hz, 3H), 1.29 (br s, 6H).

### Example 3

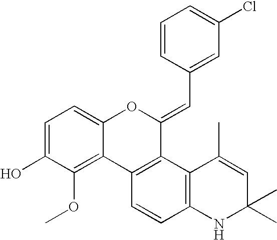

(Z)-5-(3′-chlorobenzylidene)-1,2-dihydro-9-hydroxy-10-methoxy-2,2,4-trimethyl-5H-chromeno[3,4-f]quinoline (Compound 13, structure 1 of Scheme I, where R¹=3-chlorophenyl)

This compound was prepared according to General Method 1 (Example 1) from 3-chlorobenzyl chloride. ¹H NMR (400 MHz, CD₃OD) δ 8.28 (d, J=8.6 Hz), 7.42 (s, 1H), 7.27-7.18 (m, 2H), 6.82-6.70 (m, 4H), 5.54 (s, 1H), 5.52 (s, 1H), 3.76 (s, 3H), 2.06 (s, 3H), 1.31 (br s, 6H).

### Example 4

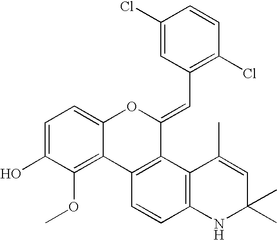

(Z)-5-(2′,5′-dichlorobenzylidene)-1,2-dihydro-9-hydroxy-10-methoxy-2,2,4-trimethyl-5H-chromeno[3,4-f]quinoline (Compound 14, structure 1 of Scheme I, where R¹=2,5-dichlorophenyl).

This compound was prepared according to General Method 1 (Example 1) from 2,5-dichlorobenzyl chloride. ¹H NMR (400 MHz, CD₃OD) δ 8.39-8.32 (m, 2-overlapping signals, 2H), 7.39 (d, J=8.6 Hz, 1H), 7.19 (d, J=8.4 Hz, 1H), 6.81-6.75 (m, 3H), 6.07 (s, 1H), 5.53 (s, 1H), 3.79 (s, 3H), 2.09 (s, 3H), 1.31 (br s, 6H).

### Example 5

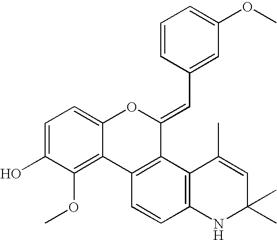

(Z)-5-(3′-methoxybenzylidene)-1,2-dihydro-9-hydroxy-10-methoxy-2,2,4-trimethyl-5H-chromeno[3,4-f]quinoline (Compound 15, structure 1 of Scheme I, where R¹=3-methoxyphenyl)

This compound was prepared according to General Method 1 (Example 1) from 3-methoxybenzyl bromide. ¹H NMR (400 MHz, CD₃OD) δ 8.28 (d, J=8.6 Hz), 7.42 (s, 1H), 7.27-7.18 (m, 2H), 6.82-6.70 (m, 4H), 5.54 (s, 1H), 5.52 (s, 1H), 3.84 (s, 3H), 3.76 (s, 3H), 2.06 (s, 3H), 1.31 (br s, 6H).

### Example 6

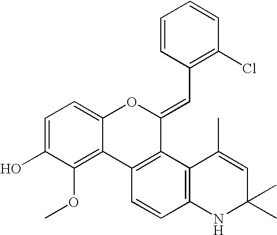

(Z)-5-(2′-chlorobenzylidene)-1,2-dihydro-9-hydroxy-10-methoxy-2,2,4-trimethyl-5H-chromeno[3,4-f]quinoline (Compound 16, structure 1 of Scheme I, where R¹=2-chlorophenyl)

This compound was prepared according to General Method 1 (Example 1) from 2-chlorobenzyl chloride. ¹H NMR (400 MHz, acetone-d₆) δ 8.40 (dd, J=2.9, 2.9 Hz, 1H), 8.32 (d, J=7.2 Hz, 1H), 7.40 (m, 1H), 7.22 (m, 1H), 6.84 (m, 2H), 6.84 (d, J=8.6 Hz, 1H), 6.15 (s, 1H), 5.91 (s, 1H), 5.61 (s, 1H), 5.52 (s, 1H), 3.77 (s, 3H), 2.08 (s, 3H), 1.35 (br s, 6H).

### Example 7

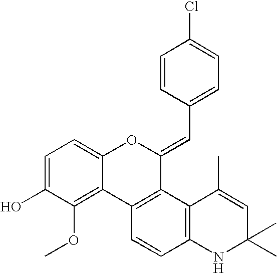

(Z)-5-(4′-chlorobenzylidene)-1,2-dihydro-9-hydroxy-10-methoxy-2,2,4-trimethyl-5H-chromeno[3,4-f]quinoline (Compound 17, structure 1 of Scheme I, where R¹=4-chlorophenyl)

This compound was prepared according to General Method 1 (Example 1) from 4-chlorobenzyl bromide. ¹H NMR (400 MHz, acetone-d₆) δ 8.29 (d, J=7.2 Hz, 1H), 7.80 (m, 2H), 7.75 (s, 1H), 7.37 (m, 2H), 6.92 (m, 1H), 6.67 (m, 2H), 5.87 (s, 1H), 5.64 (s, 1H), 5.21 (s, 1H), 3.77 (s, 3H), 2.08 (s, 3H), 1.30 (br s, 6H).

### Example 8

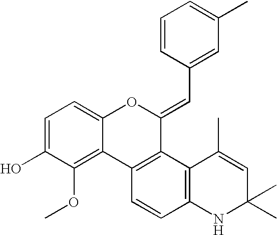

(Z)-5-(3′-methylbenzylidene)-1,2-dihydro-9-hydroxy-10-methoxy-2,2,4-trimethyl-5H-chromeno[3,4-f]quinoline (Compound 18, structure 1 of Scheme I, where R¹=3-methylphenyl)

This compound was prepared according to General Method 1 (Example 1) from 3-methylbenzyl bromide. ¹H NMR (400 MHz, acetone-d₆) δ 8.28 (d, J=7.2 Hz, 1H), 7.74 (s, 1H), 7.60 (m, 2H), 7.16 (m, 2H), 6.87 (m, 1H), 6.77 (m, 1H), 5.83 (br s, 1H), 5.61 (s, 1H), 5.51 (br s, 1H), 3.76 (s, 3H), 2.34 (s, 3H), 2.08 (s, 3H), 1.30 (br s, 6H).

### Example 9

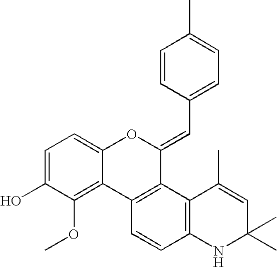

(Z)-5-(4′-methylbenzylidene)-1,2-dihydro-9-hydroxy-10-methoxy-2,2,4-trimethyl-5H-chromeno[3,4-f]quinoline (Compound 19, structure 1 of Scheme I, where R¹=4-methylphenyl)

This compound was prepared according to General Method 1 (Example 1) from 4-methylbenzyl bromide. ¹H NMR (400 MHz, CD₃OD) δ 8.25 (d, J=8.9 Hz, 1H), 7.60-7.53 (m, 2-overlapping signals, 2H), 7.10-7.08 (m, 2-overlapping signals, 2H), 6.78 (d, J=8.6 Hz, 1H), 6.72-6.68 (m, 2-overlapping signals, 2H), 5.50 (s, 1H), 5.46 (s, 1H), 3.72 (s, 3H), 2.29 (s, 3H), 2.02 (s, 3H), 1.25 (br s, 6H).

### Example 10

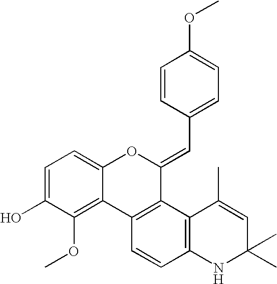

(Z)-5-(4′-methoxybenzylidene)-1,2-dihydro-9-hydroxy-10-methoxy-2,2,4-trimethyl-5H-chromeno[3,4-f]quinoline (Compound 20, structure 1 of Scheme I, where R¹=4-methoxyphenyl)

This compound was prepared according to General Method 1 (Example 1) from 4-methoxybenzyl bromide. ¹H NMR (400 MHz, CD₃OD) δ 8.24 (d, J=8.6 Hz, 1H), 7.68-7.55 (m, 2-overlapping signals, 2H), 6.92-6.86 (m, 2-overlapping signals, 2H), 6.78 (d, J=8.6 Hz, 1H), 6.72-6.66 (m, 2-overlapping signals, 2H), 5.50-5.47 (m, 2-overlapping signals, 2H), 3.78 (s, 3H), 3.73 (s, 3H), 2.03 (s, 3H), 1.29 (br s, 6H).

### Example 11

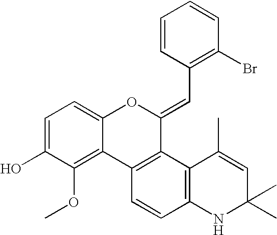

(Z)-5-(2′-bromobenzylidene)-1,2-dihydro-9-hydroxy-10-methoxy-2,2,4-trimethyl-5H-chromeno[3,4-f]quinoline (Compound 21, structure 1 of Scheme I, where R¹=2-bromophenyl)

This compound was prepared according to General Method 1 (Example 1) from 2-bromobenzyl bromide. ¹H NMR (400 MHz, acetone-d₆) δ 8.41 (dd, J=7.2, 7.2 Hz, 1H), 8.33 (d, J=6.9 Hz, 1H), 7.80 (s, 1H), 7.61 (dd, J=8.0, 1.1 Hz, 1H), 7.43 (m, 1H), 7.14 (m, 1H), 6.86 (m, 2H), 6.78 (m, 1H), 6.15 (s, 1H), 5.92 (br s, 1H), 5.51 (br s, 1H), 3.80 (s, 3H), 2.08 (s, 3H), 1.31 (br s, 6H).

### Example 12

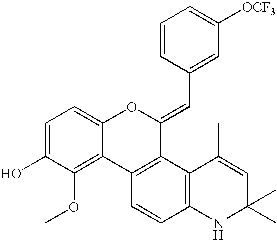

(Z)-5-(3′-trifluoromethoxybenzylidene)-1,2-dihydro-9-hydroxy-10-methoxy-2,2,4-trimethyl-5H-chromeno[3,4-f]quinoline (Compound 22, structure 1 of Scheme I, where R¹=3-trifluoromethoxyphenyl)

This compound was prepared according to General Method 1 (Example 1) from 3-trifluoromethoxybenzyl bromide. ¹H NMR (400 MHz, acetone-d₆) δ 8.31 (d, J=7.2 Hz, 1H), 7.88 (s, 1H), 7.81 (s, 1H), 7.65 (m, 1H), 7.47 (m, 1H), 7.16 (m, 1H), 6.84 (m, 2H), 5.91 (br s, 1H), 5.71 (s, 1H), 5.27 (s, 1H), 3.79 (s, 3H), 2.08 (s, 3H), 1.32 (br s, 6H).

### Example 13

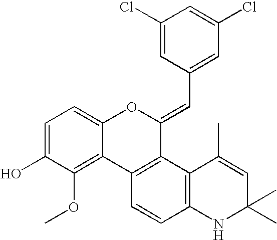

(Z)-5-(3′,5′-dichlorobenzylidene)-1,2-dihydro-9-hydroxy-10-methoxy-2,2,4-trimethyl-5H-chromeno[3,4-f]quinoline (Compound 23, structure 1 of Scheme I, where R¹=3,5-dichlorophenyl)

This compound was prepared according to General Method 1 (Example 1) from 3,5-dichlorobenzyl chloride. ¹H NMR (400 MHz, CD₃OD) δ 8.33 (d, J=8.8 Hz, 1H), 7.67 (d, J=1.8 Hz, 1H), 7.23 (t, J=1.8 Hz, 1H), 6.83-6.75 (m, 4H), 5.53 (s, 1H), 5.52 (s, 1H), 3.77 (s, 3H), 2.04 (s, 3H), 1.31 (br s, 6H).

### Example 14

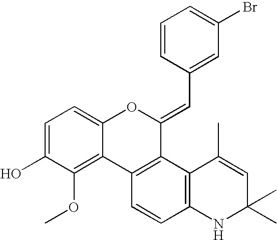

(Z)-5-(3′-bromobenzylidene)-1,2-dihydro-9-hydroxy-10-methoxy-2,2,4-trimethyl-5H-chromeno[3,4-f]quinoline (Compound 24, structure 1 of Scheme I, where R¹=3-bromophenyl)

This compound was prepared according to General Method 1 (Example 1) from 3-bromobenzyl bromide. ¹H NMR (400 MHz, acetone-d₆) δ 8.32 (d, J=7.2 Hz, 1H), 8.01 (s, 1H), 7.80 (s, 1H), 7.80 (m, 1H), 7.38 (m, 1H), 7.31 (m, 1H), 6.86 (m, 2H), 6.78 (m, 1H), 5.64 (s, 1H), 5.50 (s, 1H), 3.79 (s, 3H), 2.08 (s, 3H), 1.32 (br s, 6H).

### Example 15

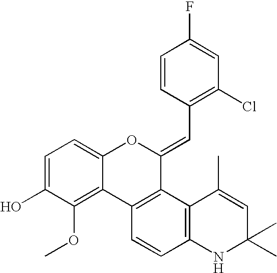

(Z)-5-(2′-chloro-4′-fluorobenzylidene)-1,2-dihydro-9-hydroxy-10-methoxy-2,2,4-trimethyl-5H-chromeno[3,4-f]quinoline (Compound 25, structure 1 of Scheme I, where R¹=2-chloro-4-fluorophenyl)

This compound was prepared according to General Method 1 (Example 1) from 2-chloro-4-fluorobenzyl bromide. ¹H NMR (400 MHz, CD₃OD) δ 8.35-8.28 (m, 2H), 7.18 (dd, J=8.8, 2.7 Hz, 1H), 7.11 (dt, J=8.5, 2.6 Hz, 1H), 6.81-6.73 (m, 2-overlapping signals, 2H), 6.70 (d, J=8.7 Hz, 1H), 6.01 (s, 1H), 5.48 (s, 1H), 3.75 (s, 3H), 2.06 (s, 3H), 1.28 (br s, 6H).

### Example 16

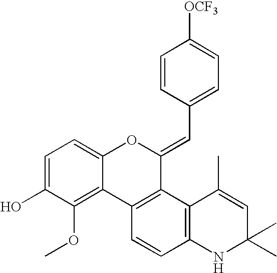

(Z)-5-(4′-trifluoromethoxybenzylidene)-1,2-dihydro-9-hydroxy-10-methoxy-2,2,4-trimethyl-5H-chromeno[3,4-f]quinoline (Compound 26, structure 1 of Scheme I, where R¹=4-trifluoromethoxyphenyl)

This compound was prepared according to General Method 1 (Example 1) from 4-trifluoromethoxybenzyl bromide. ¹H NMR (400 MHz, acetone-d₆) δ 8.30 (d, J=7.2 Hz, 1H), 7.90 (m, 2H), 7.32 (m, 1H), 6.92 (d, J=5.5 Hz, 1H), 6.79 (m, 2H), 5.69 (br s, 1H), 5.51 (s, 1H), 5.27 (s, 1H), 3.76 (s, 3H), 2.08 (s, 3H), 1.32 (br s, 6H).

### Example 17

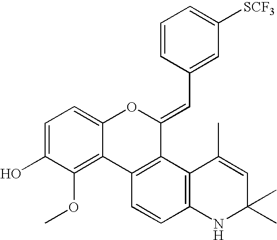

(Z)-5-(3′-trifluoromethylthiobenzylidene)-1,2-dihydro-9-hydroxy-10-methoxy-2,2,4-trimethyl-5H-chromeno[3,4-f]quinoline (Compound 27, structure 1 of Scheme I, where R¹=3-trifluoromethylthiophenyl)

This compound was prepared according to General Method 1 (Example 1) from 3-trifluoromethylthiobenzyl bromide. ¹H NMR (400 MHz, CD₃OD) δ 8.30 (d, J=8.7 Hz, 1H), 8.16 (s, 1H), 7.74 (d, J=7.6 Hz, 1H), 7.50-7.42 (m, 2H), 6.81-6.74 (m, 3H), 5.59 (s, 1H), 5.51 (s, 1H), 3.75 (s, 3H), 2.05 (s, 3H), 1.30 (br s, 6H).

### Example 18

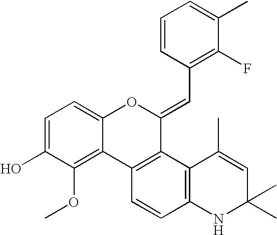

(Z)-5-(2′-fluoro-3′-methylbenzylidene)-1,2-dihydro-9-hydroxy-10-methoxy-2,2,4-trimethyl-5H-chromeno[3,4-f]quinoline (Compound 28, structure 1 of Scheme I, where R¹=2-fluoro-3-methylphenyl)

This compound was prepared according to General Method 1 (Example 1) from 2-fluoro-3-methylbenzyl bromide. ¹H NMR (400 MHz, CD₃OD) δ 8.29 (d, J=8.7 Hz, 1H), 8.11-8.02 (m, 1H), 7.08-6.99 (m, 2H), 6.79-6.70 (m, 3H), 5.84 (s, 1H), 5.49 (s, 1H), 3.75 (s, 3H), 2.23 (s, 3H), 2.05 (s, 3H), 1.29 (br s, 6H).

### Example 19

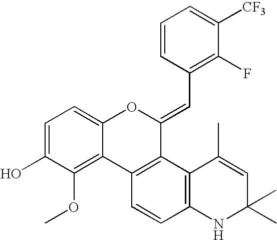

(Z)-5-(2′-fluoro-3′-trifluoromethylbenzylidene)-1,2-dihydro-9-hydroxy-10-methoxy-2,2,4-trimethyl-5H-chromeno[3,4-f]quinoline (Compound 29, structure 1 of Scheme I, where R¹=2-fluoro-3-trifluoromethylphenyl)

This compound was prepared according to General Method 1 (Example 1) from 2-fluoro-3-trifluoromethylbenzyl bromide. ¹H NMR (500 MHz, CD₃OD) 8.55 (t, J=7.0 Hz, 1H), 8.36 (d, J=8.6 Hz, 1H), 7.48 (t, J=7.0 Hz, 1H), 7.36 (t, J=7.9 Hz, 1H), 6.80-6.85 (m, 2H), 6.76 (d, J=8.6 Hz, 1H), 5.87 (s, 1H), 5.55 (s, 1H), 3.78 (s, 3H), 2.07 (s, 3H), 1.32 (br s, 6H).

### Example 20

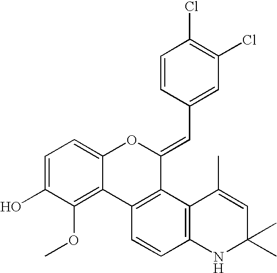

(Z)-5-(3′,4′-dichlorobenzylidene)-1,2-dihydro-9-hydroxy-10-methoxy-2,2,4-trimethyl-5H-chromeno[3,4-f]quinoline (Compound 30, structure 1 of Scheme I, where R¹=3,4-dichlorophenyl)

This compound was prepared according to General Method 1 (Example 1) from 3,4-dichlorobenzyl chloride. ¹H NMR (500 MHz, CDCl₃) δ 8.25 (m, 1H), 8.17 (d, J=8.8 Hz, 1H), 7.19 (m, 2H), 6.85 (d, J=8.8 Hz, 1H), 6.69 (d, J=8.3 Hz, 1H), 6.74 (d, J=8.8 Hz, 1H), 5.92 (s, 1H), 5.53 (s, 1H), 3.78 (s, 3H), 2.12 (d, J=1.0 Hz, 3H), 1.29 (br s, 6H).

### Example 21

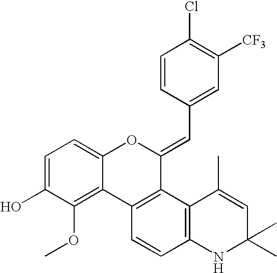

(Z)-5-(4′-chloro-3′-trifluoromethylbenzylidene)-1,2-dihydro-9-hydroxy-10-methoxy-2,2,4-trimethyl-5H-chromeno[3,4-f]quinoline (Compound 31, structure 1 of Scheme I, where R¹=4-chloro-3-trifluoromethylphenyl)

This compound was prepared according to General Method 1 (Example 1) from 4-chloro-3-trifluoromethylbenzyl bromide. ¹H NMR (500 MHz, CD₃OD) δ 8.55 (d, J=7.1 Hz, 1H), 8.36 (d, J=8.7 Hz, 1H), 8.19 (s, 1H), 7.48 (t, J=7.2 Hz, 1H), 7.36 (t, J=7.9 Hz, 1H), 6.82 (m, 2H), 6.76 (s, 1H), 5.56 (s, 1H), 3.74 (s, 3H), 2.16 (s, 3H), 1.36 (br s, 6H).

### Example 22

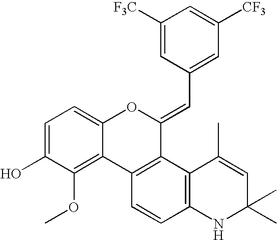

(Z)-5-(3′,5′-di(trifluoromethyl)benzylidene)-1,2-dihydro-9-hydroxy-10-methoxy-2,2,4-trimethyl-5H-chromeno[3,4-f]quinoline (Compound 32, structure 1 of Scheme I, where R¹=3,5-di(trifluoromethyl)phenyl)

This compound was prepared according to General Method 1 (Example 1) from 3,5-di(trifluoromethyl)benzyl bromide. ¹H NMR (500 MHz, CD₃OD) δ 8.36 (d, J=8.9 Hz, 1H), 8.29 (s, 2H), 7.74 (s, 1H), 6.82 (d, J=8.6 Hz, 1H), 6.77 (s, 2H), 5.76 (s, 1H), 5.56 (s, 1H), 3.78 (s, 3H), 2.07 (s, 3H), 1.33 (br s, 6H).

### Example 23

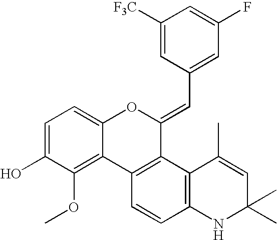

(Z)-5-(3′-fluoro-5′-trifluoromethylbenzylidene)-1,2-dihydro-9-hydroxy-10-methoxy-2,2,4-trimethyl-5H-chromeno[3,4-f]quinoline (Compound 33, structure 1 of Scheme I, where R¹=3-fluoro-5-trifluoromethylphenyl)

This compound was prepared according to General Method 1 (Example 1) from 3-fluoro-5-trifluoromethylbenzyl bromide. ¹H NMR (500 MHz, CD₃OD) δ 8.35 (d, J=8.9 Hz, 1H), 7.84 (s, 1H), 7.74 (d, J=10.4 Hz, 1H), 7.24 (d, J=8.6 Hz, 1H), 6.82 (d, J=3.4 Hz, 1H), 6.80 (d, J=3.1 Hz, 1H), 6.78 (d, J=8.9 Hz, 1H), 5.66 (s, 1H), 5.43 (s, 1H), 3.77 (s, 3H), 2.05 (s, 3H), 1.32 (br s, 6H).

### Example 24

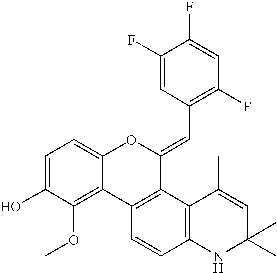

(Z)-5-(2′,4′,5′-trifluorobenzylidene)-1,2-dihydro-9-hydroxy-10-methoxy-2,2,4-trimethyl-5H-chromeno[3,4-f]quinoline (Compound 34, structure 1 of Scheme I, where R¹=2,4,5-trifluorophenyl)

This compound was prepared according to General Method 1 (Example 1) from 2,4,5-trifluorobenzyl bromide. ¹H NMR (500 MHz, CDCl₃) δ 8.17-8.20 (m, 2H), 6.85-6.93 (m, 3H), 6.71 (d, J=8.6 Hz, 1H), 5.54 (s, 1H), 4.72 (s, 1H), 3.80 (s, 3H), 2.08 (s, 3H), 1.35 (br s, 6H).

### Example 25

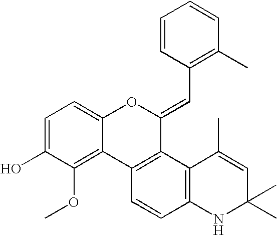

(Z)-5-(2′-methylbenzylidene)-1,2-dihydro-9-hydroxy-10-methoxy-2,2,4-trimethyl-5H-chromeno[3,4-f]quinoline (Compound 35, structure 1 of Scheme I, where R¹=2-methylphenyl)

This compound was prepared according to General Method 1 (Example 1) from 2-methylbenzyl bromide. ¹H NMR (400 MHz, CDCl₃) δ 8.19 (m, 2H), 7.25 (m, 2H), 7.18 (m, 2H), 6.85-6.93 (m, 2H), 6.71 (m, 1H), 5.86 (s, 1H), 5.52 (m, 1H), 5.30 (s, 1H), 3.80 (s, 3H), 2.28 (s, 3H), 2.14 (s, 3H), 1.35 (br s, 6H).

### Example 26

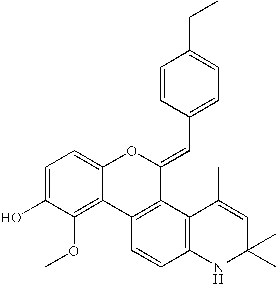

(Z)-5-(4′-ethylbenzylidene)-1,2-dihydro-9-hydroxy-10-methoxy-2,2,4-trimethyl-5H-chromeno[3,4-f]quinoline (Compound 36, structure 1 of Scheme I, where R¹=4-ethylphenyl)

This compound was prepared according to General Method 1 (Example 1) from 4-ethylbenzyl bromide. ¹H NMR (500 MHz, CD₃OD) δ 8.27 (d, J=8.6 Hz), 7.63 (d, J=8.2 Hz, 2H), 7.18 (d, J=8.2 Hz, 2H), 6.81 (d, J=8.9 Hz, 1H), 6.74 (d, J=8.9 Hz, 1H), 6.72 (d, J=8.9 Hz, 1H), 5.53 (s, 1H), 5.50 (s, 1H), 3.75 (s, 3H), 2.63 (q, J=7.63 Hz, 2H), 2.05 (s, 3H), 1.30 (br s, 6H), 1.24 (t, J=7.63 Hz, 3H).

### Example 27

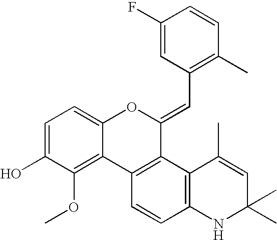

(Z)-5-(5′-fluoro-2′-methylbenzylidene)-1,2-dihydro-9-hydroxy-10-methoxy-2,2,4-trimethyl-5H-chromeno[3,4-f]quinoline (Compound 37, structure 1 of Scheme I, where R¹=5-fluoro-2-methylphenyl)

This compound was prepared according to General Method 1 (Example 1) from 5-fluoro-2-methylbenzyl bromide. ¹H NMR (500 MHz, CDCl₃) δ 8.23 (s, 1H), 8.03 (m, 1H), 7.95 (m, 1H), 7.88 (m, 1H), 7.67 (m, 1H), 7.60 (m, 2H), 7.55 (m, 1H), 6.92 (d, 1H), 5.30 (s, 1H), 4.92 (s, 1H), 3.79 (s, 3H) 2.36 (s, 3H), 2.08 (s, 3H), 1.32 (br s, 6H).

### Example 28

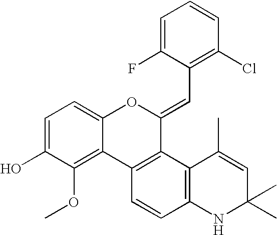

(Z)-5-(2′-chloro-6′-fluorobenzylidene)1,2-dihydro-9-hydroxy-10-methoxy-2,2,4-trimethyl-5H-chromeno[3,4-f]quinoline (Compound 38, structure 1 of Scheme I, where R¹=2-chloro-6-fluorophenyl)

This compound was prepared according to General Method 1 (Example 1) from 2-chloro-6-fluorobenzyl bromide. ¹H NMR (400 MHz, CD₃OD) δ 8.33 (d, J=8.8 Hz, 1H), 7.27-7.20 (m, 2H), 7.12-7.07 (m, 1H), 6.78 (d, J=8.7 Hz, 1H), 6.62 (d, J=8.6 Hz, 1H), 6.47 (d, J=8.8 Hz, 1H), 5.55 (s, 1H), 5.49 (s, 1H), 3.76 (s, 3H), 2.18 (s, 3H), 1.28 (br s, 6H).

### Example 29

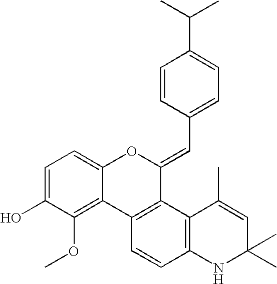

(Z)-5-(4′-isopropylbenzylidene)-1,2-dihydro-9-hydroxy-10-methoxy-2,2,4-trimethyl-5H-chromeno[3,4-f]quinoline (Compound 39, structure 1 of Scheme I, where R¹=4-isopropylphenyl)

This compound was prepared according to General Method 1 (Example 1) from 4-isopropylbenzyl bromide. ¹H NMR (500 MHz, CD₃OD) δ 8.27 (d, J=8.6 Hz, 1H), 7.64 (d, J=8.2 Hz, 2H), 7.21 (d, J=8.2 Hz, 1H), 6.82 (d, J=8.6 Hz, 1H), 6.74 (d, J=8.6 Hz, 1H), 6.72 (d, J=8.9 Hz, 1H), 5.53 (s, 1H), 5.50 (s, 1H), 3.75 (s, 3H), 2.89 (septet, J=7.0 Hz, 1H), 2.05 (s, 3H), 1.30 (br s, 6H), 1.25 (d, J=6.7 Hz, 1H).

### Example 30

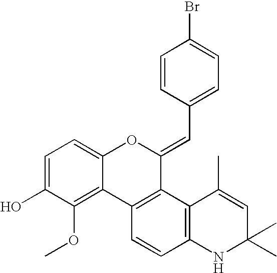

(Z)-5-(4′-bromobenzylidene)-1,2-dihydro-9-hydroxy-10-methoxy-2,2,4-trimethyl-5H-chromeno[3,4-f]quinoline (Compound 40, structure 1 of Scheme I, where R¹=4-bromophenyl)

This compound was prepared according to General Method 1 (Example 1) from 4-bromobenzyl bromide. ¹H NMR (400 MHz, CD₃OD) δ 8.27 (d, J=8.6 Hz, 1H), 7.65-7.58 (m, 2-overlapping signals, 2H), 7.49-7.41 (m, 2-overlapping signals, 2H), 6.81 (d, J=8.7 Hz, 1H), 6.74 (d, J=8.6 Hz, 1H), 6.71 (d, J=8.8 Hz, 1H), 5.51 (s, 1H), 5.49 (s, 1H), 3.74 (s, 3H), 2.02 (s, 3H), 1.28 (br s, 6H).

### Example 31

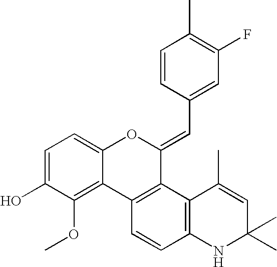

(Z)-5-(3′-fluoro-4′-methylbenzylidene)-1,2-dihydro-9-hydroxy-10-methoxy-2,2,4-trimethyl-5H-chromeno[3,4-f]quinoline (Compound 41, structure 1 of Scheme I, where R¹=3-fluoro-4-methylphenyl)

This compound was prepared according to General Method 1 (Example 1) from 3-fluoro-4-methylbenzyl bromide. ¹H NMR (500 MHz, CD₃OD) δ 8.30 (d, J=8.9 Hz, 1H), 7.54 (dd, J=11.9, 1.2 Hz, 1H), 7.28 (d, J=8.2, 1.5 Hz, 1H), 7.18 (t, J=8.1 Hz, 1H), 6.83 (d, J=8.9 Hz, 1H), 6.76 (m, 2H), 5.53 (m, 2H), 3.76 (s, 3H), 2.26 (s, 3H), 2.04 (s, 3H), 1.31 (br s, 6H).

### Example 32

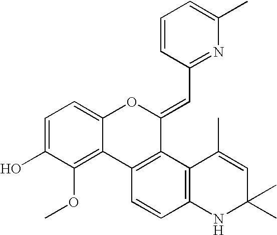

(Z)-5-(2′-(6′-methyl-pyridinylmethylidiene))-1,2-dihydro-9-hydroxy-10-methoxy-2,2,4-trimethyl-5H-chromeno[3,4-f]quinoline (Compound 42, structure 1 of Scheme I, where R¹=2-(6-methylpyridinyl))

This compound was prepared according to General Method 2 (Example 60) from 2,6-lutidine. ¹H NMR (500 MHz, CD₃OD) δ 8.34 (d, J=8.5 Hz, 1H), 8.24 (d, J=8.2 Hz, 1H), 7.74 (t, J=7.8 Hz, 1H), 7.07 (d, J=7.7 Hz, 1H), 6.87 (d, J=8.9 Hz, 1H), 6.80 (d, J=8.5 Hz, 1H), 6.74 (d, J=8.6 Hz, 1H), 5.87 (s, 1H), 5.52 (d, J=1.2 Hz, 1H), 3.77 (s, 3H), 2.48 (s, 3H), 2.07 (d, J=1.2 Hz, 3H), 1.33 (br s, 6H).

### Example 33

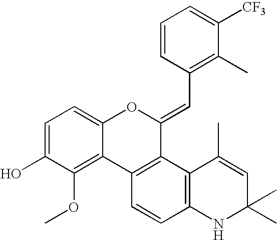

(Z)-5-(2′-methyl-3′-trifluoromethylbenzylidene)-1,2-dihydro-9-hydroxy-10-methoxy-2,2,4-trimethyl-5H-chromeno[3,4-f]quinoline (Compound 43, structure 1 of Scheme I, where R¹=2-methyl-3-trifluoromethylphenyl)

This compound was prepared according to General Method 1 (Example 1) from 2-methyl-3-trifluoromethylbenzyl bromide. ¹H NMR (500 MHz, CD₃OD) δ 8.33 (d, J=8.6 Hz, 1H), 8.24 (d, J=7.9 Hz, 1H), 7.51 (d, J=7.9 Hz, 1H), 7.37 (t, J=7.9 Hz, 1H), 6.79 (d, J=8.6 Hz, 1H), 6.70 (d, J=8.6 Hz, 1H), 6.66 (d, J=8.9 Hz, 1H), 5.88 (s, 1H), 5.52 (s, 1H), 3.77 (s, 3H), 2.33 (s, 3H), 2.11 (s, 3H), 1.31 (br s, 6H).

### Example 34

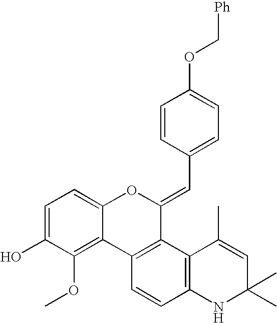

(Z)-5-(4′-benzyloxybenzylidene)-1,2-dihydro-9-hydroxy-10-methoxy-2,2,4-trimethyl-5H-chromeno[3,4-f]quinoline (Compound 44, structure 1 of Scheme I, where R¹=4-benzyloxyphenyl)

This compound was prepared according to General Method 1 (Example 1) from 4-benzyloxybenzyl bromide. ¹H NMR (500 MHz, CDCl₃) δ 8.14 (d, J=8.6 Hz, 1H), 7.72 (d, J=8.9 Hz, 2H), 7.45 (d, J=7.0 Hz, 2H), 7.40 (dd, J=7.6, 7.0 Hz, 2H), 7.34 (d, J=7.6 Hz, 1H), 6.98 (d, J=8.9 Hz, 2H), 6.89 (d, J=8.9 Hz, 1H), 6.81 (d, J=8.9 Hz, 1H), 6.65 (d, J=8.6 Hz, 1H), 5.56 (s, 1H), 5.31 (s, 1H), 5.10 (s, 2H), 3.78 (s, 3H), 2.10 (s, 3H), 1.35 (br s, 6H).

### Example 35

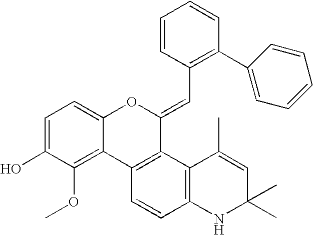

(Z)-5-(2′-phenylbenzylidene)-1,2-dihydro-9-hydroxy-10-methoxy-2,2,4-trimethyl-5H-chromeno[3,4-f]quinoline (Compound 46, structure 1 of Scheme I, where R¹=2-biphenyl)

This compound was prepared according to General Method 1 (Example 1) from 2-phenylbenzyl bromide. ¹H NMR (500 MHz, CD₃OD) δ 8.51 (d, J=7.1 Hz, 1H), 8.25 (d, J=7.3 Hz, 1H), 7.42 (m, 1H), 7.38 (m, 2H), 7.22 (m, 4H), 6.82 (d, 1H), 6.72 (d, J=7.2 Hz, 1H), 6.67 (d, 1H), 5.65 (s, 1H), 5.25 (s, 1H), 3.76 (s, 3H), 1.88 (s, 3H), 2.25 (s, 3H), 1.32 (br s, 6H).

### Example 36

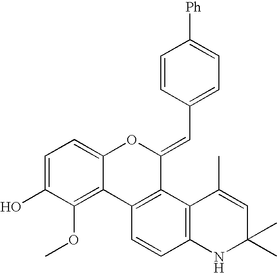

(Z)-5-(4′-phenylbenzylidene)-1,2-dihydro-9-hydroxy-10-methoxy-2,2,4-trimethyl-5H-chromeno[3,4-f]quinoline (Compound 47, structure 1 of Scheme I, where R¹=4-biphenyl)

This compound was prepared according to General Method 1 (Example 1) from 4-phenylbenzyl bromide. ¹H NMR (500 MHz, CD₃OD) δ 8.31 (d, J=8.6 Hz, 1H), 7.81 (d, J=8.2 Hz, 2H), 6.65 (d, J=8.6 Hz, 2H), 7.63 (d, J=8.6 Hz, 2H), 7.42 (dd, J=8.2, 7.3 Hz, 2H), 7.31 (t, J=7.30, 1H), 6.87 (d, J=8.9 Hz, 1H), 6.78 (d, J=8.6 Hz, 1H), 6.75 (d, J=8.9 Hz, 1H), 5.61 (s, 1H), 5.53 (s, 1H), 3.77 (s, 3H), 2.08 (s, 3H), 1.32 (br s, 6H).

### Example 37

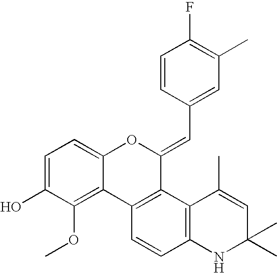

(Z)-5-(3′-methyl-4′-fluorobenzylidene)-1,2-dihydro-9-hydroxy-10-methoxy-2,2,4-trimethyl-5H-chromeno[3,4-f]quinoline (Compound 48, structure 1 of Scheme I, where R¹=4-fluoro-3-methylphenyl)

This compound was prepared according to General Method 1 (Example 1) from 4-fluoro-3-methylbenzyl bromide. ¹H NMR (500 MHz, CDCl₃) δ 8.15 (d, J=7.1 Hz, 1H), 7.52 (m, 2H), 6.97 (m, 1H), 6.88 (m, 1H), 6.68 (d, 1H), 5.55 (m, 3H), 4.18 (m, 1H), 3.78 (s, 3H), 2.31 (s, 3H), 2.09 (s, 3H), 1.88 (s, 3H), 2.25 (s, 3H), 1.48 (br s, 6H).

### Example 38

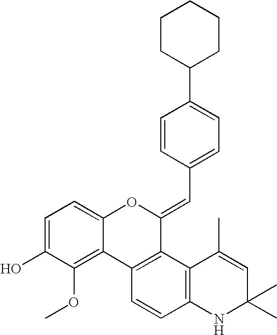

(Z)-5-(4′-cyclohexylbenzylidene)-1,2-dihydro-9-hydroxy-10-methoxy-2,2,4-trimethyl-5H-chromeno[3,4-f]quinoline (Compound 49, structure 1 of Scheme I, where R¹=4-cyclohexylphenyl)

This compound was prepared according to General Method 1 (Example 1) from 4-cyclohexylbenzyl bromide. ¹H NMR (500 MHz, CD₃OD) δ 8.27 (d, J=8.6 Hz, 1H), 7.62 (d, J=8.2 Hz, 2H), 7.18 (d, J=8.2 Hz, 2H), 6.82 (d, J=8.9 Hz, 1H), 6.74 (d, J=9.5 Hz, 1H), 6.72 (d, J=8.9 Hz, 1H), 5.53 (s, 1H), 5.50 (s, 1H), 3.75 (s, 3H), 2.50-2.46 (m, 1H), 2.05 (s, 3H), 1.84 (d, J=9.2 Hz, 4H), 1.75 (d, J=12.8 Hz, 1H), 1.38-1.49 (m, 5H), 1.31 (br s, 6H).

### Example 39

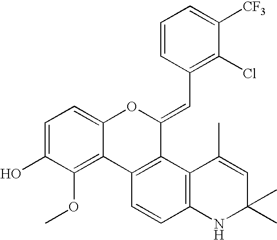

(Z)-5-(2′-chloro-3′-trifluoromethylbenzylidene)-1,2-dihydro-9-hydroxy-10-methoxy-2,2,4-trimethyl-5H-chromeno[3,4-f]quinoline (Compound 51, structure 1 of Scheme I, where R¹=2-chloro-3-trifluoromethylphenyl)

This compound was prepared according to General Method 1 (Example 1) from 2-chloro-3-trifluoromethylbenzyl bromide. ¹H NMR (500 MHz, CD₃Cl) δ 8.44 (d, J=8.6 Hz, 1H), 8.21 (d, J=7.9 Hz, 1H), 7.55 (d, J=7.9 Hz, 1H), 7.39 (t, J=7.9 Hz, 1H), 6.82 (d, J=8.6 Hz, 1H), 6.72 (d, J=8.6 Hz, 1H), 6.11 (s, 1H), 5.59 (s, 1H), 5.30 (s, 1H), 3.81 (s, 3H), 2.13 (s, 3H), 1.36 (br s, 6H).

### Example 40

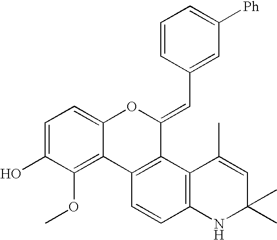

(Z)-5-(3′-phenylbenzylidene)-1,2-dihydro-9-hydroxy-10-methoxy-2,2,4-trimethyl-5H-chromeno[3,4-f]quinoline (Compound 52, structure 1 of Scheme I, where R¹=3-biphenyl)

This compound was prepared according to General Method 1 (Example 1) from 3-phenylbenzyl bromide. ¹H NMR (500 MHz, CD₃OD) δ 8.30 (d, J=8.6 Hz, 1H), 8.00 (s, 1H), 7.64-7.68 (m, 3H), 7.41-7.48 (m, 4H), 7.36 (t, J=7.3 Hz, 1H), 6.72-6.81 (m, 3H), 5.65 (s, 1H), 5.54 (s, 1H), 3.78 (s, 3H), 2.10 (s, 3H), 1.32 (br s, 6H).

### Example 41

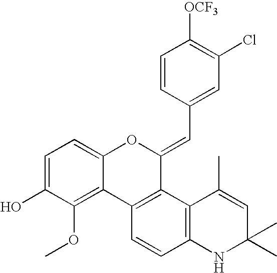

(Z)-5-(3′-chloro-4′-trifluoromethoxybenzylidene)-1,2-dihydro-9-hydroxy-10-methoxy-2,2,4-trimethyl-5H-chromeno[3,4-f]quinoline (Compound 54, structure 1 of Scheme I, where R¹=3-chloro-4-trifluoromethoxyphenyl)

This compound was prepared according to General Method 1 (Example 1) from 3-chloro-4-trifluoromethoxybenzyl bromide. ¹H NMR (500 MHz, CD₃OD) δ 8.31 (d, J=8.6 Hz, 1H), 7.95 (s, 1H), 7.68 (d, J=8.6 Hz, 1H), 7.37 (d, J=8.6 Hz, 1H), 6.82 (d, J=8.9 Hz, 1H), 6.77 (d, J=8.6 Hz, 1H), 6.74 (d, J=8.9 Hz, 1H), 5.55 (s, 1H), 5.51 (s, 1H), 3.75 (s, 3H), 2.03 (s, 3H), 1.29 (br s, 6H).

### Example 42

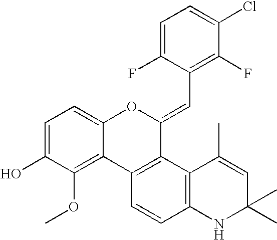

(Z)-5-(2′,6′-difluoro-3′-chlorobenzylidene)-1,2-dihydro-9-hydroxy-10-methoxy-2,2,4-trimethyl-5H-chromeno[3,4-f]quinoline (Compound 55, structure 1 of Scheme I, where R¹=3′-chloro-2,6-difluorophenyl)

This compound was prepared according to General Method 1 (Example 1) from 3-chloro-2,6-difluorobenzyl bromide. ¹H NMR (500 MHz, CD₃OD) δ 8.37 (d, J=8.6 Hz, 1H), 7.35-7.40 (m, 1H), 6.99 (dt, J=8.9, 1.8 Hz, 1H), 6.81 (d, J=8.9 Hz, 1H), 6.68 (d, J=8.9 Hz, 1H), 6.55 (d, J=8.9 Hz, 1H), 5.53 (s, 1H), 5.52 (s, 1H), 3.78 (s, 3H), 2.17 (s, 3H), 1.30 (br s, 6H).

### Example 43

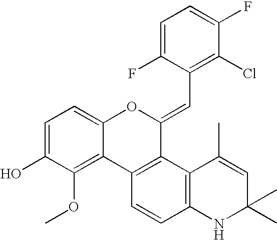

(Z)-5-(2′-chloro-3′,6′-difluorobenzylidene)-1,2-dihydro-9-hydroxy-10-methoxy-2,2,4-trimethyl-5H-chromeno[3,4-f]quinoline (Compound 56, structure 1 of Scheme I, where R¹=2-chloro-3,6-difluorophenyl)

This compound was prepared according to General Method 1 (Example 1) from 2-chloro-3,6-difluorobenzyl bromide. ¹H NMR (500 MHz, CD₃OD) δ 8.35 (d, J=8.9 Hz, 1H), 7.18-7.14 (m, 2H), 6.80 (d, J=8.6 Hz, 1H), 6.63 (d, J=8.9 Hz, 1H), 6.49 (d, J=8.6 Hz, 1H), 5.55 (s, 1H), 5.50 (s, 1H), 3.76 (s, 3H), 2.19 (s, 3H), 1.29 (br s, 6H).

### Example 44

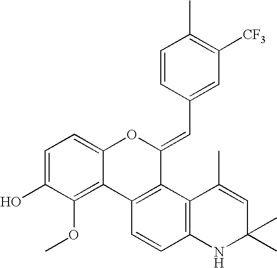

(Z)-5-(4′-methyl-3′-trifluoromethylbenzylidene)-1,2-dihydro-9-hydroxy-10-methoxy-2,2,4-trimethyl-5H-chromeno[3,4-f]quinoline (Compound 58, structure 1 of Scheme I, where R¹=4-methyl-3-trifluoromethylphenyl)

This compound was prepared according to General Method 1 (Example 1) from 4-methyl-3-trifluoromethylbenzyl bromide. ¹H NMR (500 MHz, CD₃OD) δ 8.26 (d, J=8.9 Hz, 1H), 7.47 (d, J=1.5 Hz, 1H), 7.00 (dd, J=8.4, 1.4 Hz, 1H), 6.80-6.78 (m, 2H), 6.73-6.71 (m, 2H), 5.95 (s, 2H), 5.50-5.48 (m, 2H), 3.75 (s, 3H), 2.04 (d, J=1.2 Hz, 3H), 1.30 (br s, 6H).

### Example 45

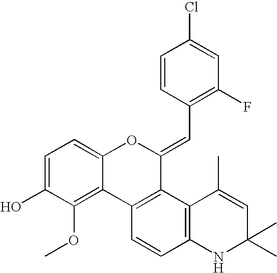

(Z)-5-(2′-fluoro-4′-chlorobenzylidene)-1,2-dihydro-9-hydroxy-10-methoxy-2,2,4-trimethyl-5H-chromeno[3,4-f]quinoline (Compound 59, structure 1 of Scheme I, where R¹=4-chloro-2-fluorophenyl)

This compound was prepared according to General Method 1 (Example 1) from 4-chloro-2-fluorobenzyl bromide. ¹H NMR (500 MHz, CDCl₃) δ 8.22 (app t, J=8.4 Hz, 1H), 8.18 (d, J=8.5 Hz, 1H), 7.16 (d, J=8.5 Hz, 1H), 7.06 (dd, J=10.4, 2.1 Hz, 1H), 6.84 (s, 1H), 6.69 (d, J=8.5 Hz, 1H), 5.83 (s, 1H), 5.70 (s, 1H), 5.53 (s, 1H), 4.22 (br s, 1H), 3.79 (s, 3H), 2.09 (s, 3H), 1.36 (br s, 6H).

### Example 46

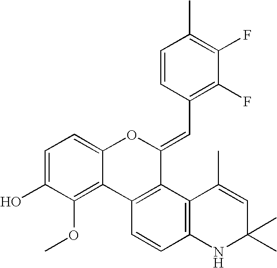

(Z)-5-(2′,3′-difluoro-4′-methylbenzylidene)-1,2-dihydro-9-hydroxy-10-methoxy-2,2,4-trimethyl-5H-chromeno[3,4-f]quinoline (Compound 60, structure 1 of Scheme I, where R¹=2,3-difluoro-4-methylphenyl)

This compound was prepared according to General Method 1 (Example 1) from 2,3-difluoro-4-methylbenzyl bromide. ¹H NMR (400 MHz, CDCl₃) δ 8.84 (d, J=7.4 Hz, 2H), 8.18 (d, J=8.4 Hz, 1H), 7.95 (m, 1H), 6.95 (m, 1H), 6.84 (s, 1H), 6.70 (d, J=8.5 Hz, 1H), 5.85 (s, 1H), 5.58 (s, 1H), 4.22 (br s, 1H), 3.79 (s, 3H), 2.31 (s, 3H), 2.10 (s, 3H), 1.36 (br s, 6H).

### Example 47

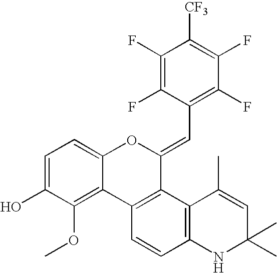

(Z)-5-(2′,3′,5′,6′-tetrafluoro-4′-trifluoromethylbenzylidene)-1,2-dihydro-9-hydroxy-10-methoxy-2,2,4-trimethyl-5H-chromeno[3,4-f]quinoline (Compound 61, structure 1 of Scheme I, where R¹=2,3,5,6-tetrafluoro-4-trifluoromethylphenyl)

This compound was prepared according to General Method 1 (Example 1) from 2,3,5,6-tetrafluoro-4-trifluoromethylbenzyl bromide. ¹H NMR (400 MHz, CD₃OD) δ 8.37 (d, J=8.6 Hz, 1H), 6.99 (dt, J=8.9, 1.8 Hz, 1H), 6.81 (d, J=8.9 Hz, 1H), 6.68 (d, J=8.9 Hz, 1H), 6.55 (d, J=8.9 Hz, 1H), 5.53 (s, 1H), 3.78 (s, 3H), 2.17 (s, 3H), 1.30 (br s, 6H).

### Example 48

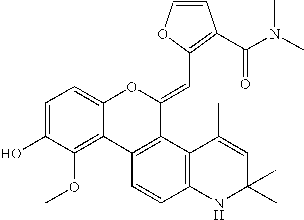

(Z)-5-(2′-(3′-(dimethylaminocarbonyl)furanylmethylidene))-1,2-dihydro-9-hydroxy-10-methoxy-2,2,4-trimethyl-5H-chromeno[3,4-f]quinoline (Compound 62, structure 1 of Scheme I, where R¹=2-(3-dimethylaminocarbonylfuranyl))

This compound was prepared according to General Method 2 (Example 60) from N,N-dimethyl-2-methyl-3-furanamide. ¹H NMR (500 MHz, CD₃OD) δ 8.35 (d, J=8.8 Hz, 1H), 7.53 (d, J=2.0 Hz, 1H), 6.80 (d, J=8.8 Hz, 1H), 6.77 (d, J=8.8 Hz, 1H), 6.71 (d, J=8.8 Hz, 1H) 6.56 (d, J=2.0 Hz, 1H), 5.65 (s, 1H), 5.51 (d, J=1.5 Hz, 1H), 3.75 (s, 3H), 3.02 (s, 6H), 2.08 (d, J=1.5 Hz, 3H), 1.29 (br s, 6H).

### Example 49

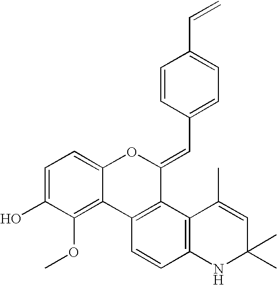

(Z)-5-(4′-vinylbenzylidene)-1,2-dihydro-9-hydroxy-10-methoxy-2,2,4-trimethyl-5H-chromeno[3,4-f]quinoline (Compound 63, structure 1 of Scheme I, where R¹=4-vinylphenyl)

This compound was prepared according to General Method 1 (Example 1) from 4-vinylbenzyl bromide. ¹H NMR (500 MHz, CD₃OD) δ 8.29 (d, J=8.6 Hz, 1H), 7.69 (d, J=8.2 Hz, 2H), 7.41 (d, J=8.6 Hz, 2H), 6.84 (d, J=8.6 Hz, 1H), 6.69-6.76 (m, 3H), 5.77 (d, J=17.7 Hz, 1H), 5.56 (s, 1H), 5.51 (s, 1H), 5.20 (d, J=11.0 Hz, 1H), 3.76 (s, 3H), 2.06 (s, 3H), 1.31 (br s, 6H).

### Example 50

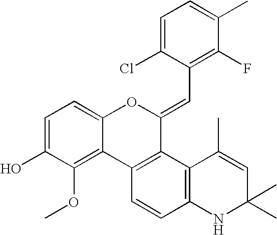

(Z)-5-(2′-Chloro-6′-fluoro-5′-methylbenzylidene)-1,2-dihydro-9-hydroxy-10-methoxy-2,2,4-trimethyl-5H-chromeno[3,4-f]quinoline (Compound 64, structure 1 of Scheme I, where R¹=2-chloro-6-fluoro-5-methylphenyl)

This compound was prepared according to General Method 1 (Example 1) from 2-chloro-6-fluoro-5-methylbenzyl bromide. ¹H NMR (500 MHz, CDCl₃) δ 8.20 (d, J=8.5 Hz, 1H), 7.26 (m, 1H, obscured by solvent), 6.96 (app t, J=8.9 Hz, 1H), 6.72 (m, 2H), 6.63 (d, J=8.9 Hz, 1H), 5.66 (s, 1H), 5.53-5.51 (m, 2H), 4.20 (br s, 1H), 3.81 (s, 3H), 2.34 (s, 2H), 2.24 (d, J=1.2 Hz, 3H), 1.35 (br s, 6H).

### Example 51

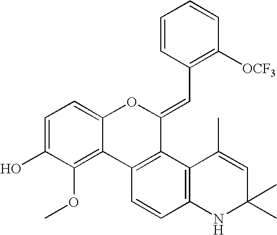

(Z)-5-(2′-trifluoromethoxybenzylidene)-1,2-dihydro-9-hydroxy-10-methoxy-2,2,4-trimethyl-5H-chromeno[3,4-f]quinoline (Compound 65, structure 1 of Scheme I, where R¹=2-trifluoromethoxyphenyl)

This compound was prepared according to General Method 1 (Example 1) from 2-trifluoromethoxybenzyl bromide. ¹H NMR (500 MHz, CD₃OD) δ 8.45 (d, J=8.8 Hz, 1H), 8.34 (d, J=8.3 Hz, 1H), 7.39 (m, 1H), 7.27-7.26 (m, 2H), 6.83 (d, J=8.8 Hz, 1H), 6.79 (d, J=8.3 Hz, 1H), 6.74 (d, J=8.8 Hz, 1H), 5.94 (s, 1H), 5.49 (d, J=1.0 Hz, 1H), 3.78 (s, 3H), 2.05 (d, J=1.0 Hz, 3H), 1.29 (br s, 6H).

### Example 52

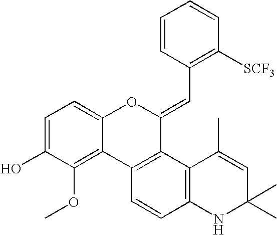

(Z)-5-(2′-trifluoromethylthiobenzylidene)-1,2-dihydro-9-hydroxy-10-methoxy-2,2,4-trimethyl-5H-chromeno[3,4-f]quinoline (Compound 66, structure 1 of Scheme I, where R¹=2-trifluoromethylthiophenyl)

This compound was prepared according to General Method 1 (Example 1) from 2-trifluoromethylthiobenzyl bromide. ¹H NMR (500 MHz, CD₃OD) δ 8.48 (d, J=8.9 Hz, 1H), 8.36 (d, J=8.6 Hz, 1H), 7.70 (d, J=8.6 Hz, 1H), 7.58 (t, J=7.6 Hz, 1H), 7.25 (t, J=7.6 Hz, 1H), 6.80-6.76 (m, 2-overlapping signals, 2H), 6.72 (d, J=8.9 Hz, 1H), 6.43 (s, 1H), 5.44 (s, 1H), 3.77 (s, 3H), 2.05 (s, 3H), 1.30 (br s, 6H).

### Example 53

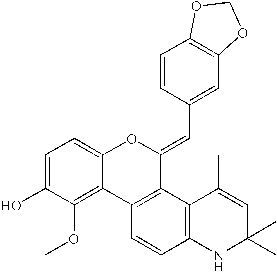

(Z)-5-(3′,4′-methylenedioxybenzylidene)-1,2-dihydro-9-hydroxy-10-methoxy-2,2,4-trimethyl-5H-chromeno[3,4-f]quinoline (Compound 67, structure 1 of Scheme I, where R¹=3,4-methylenedioxyphenyl)

This compound was prepared according to General Method 1 (Example 1) from 3,4-methylenedioxybenzyl bromide. ¹H NMR (500 MHz, CD₃OD) δ 8.26 (d, J=8.9 Hz, 1H), 7.47 (d, J=1.5 Hz, 1H), 7.00 (dd, J=8.4, 1.4 Hz, 1H), 6.80-6.78 (m, 2H), 6.73-6.71 (m, 2H), 5.95 (s, 2H), 5.50-5.48 (m, 2H), 3.75 (s, 3H), 2.04 (d, J=1.2 Hz, 3H), 1.30 (br s, 6H).

### Example 54

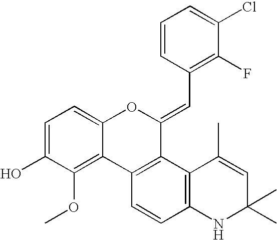

(Z)-5-(3′-chloro-2′-fluorobenzylidene)-1,2-dihydro-9-hydroxy-10-methoxy-2,2,4-trimethyl-5H-chromeno[3,4-f]quinoline (Compound 68, structure 1 of Scheme I, where R¹=3-chloro-2-fluorophenyl)

This compound was prepared according to General Method 1 (Example 1) from 3-chloro-2-fluorobenzyl bromide. ¹H NMR (500 MHz, CDCl₃) δ 8.45 (d, J=7.1 Hz, 1H), 8.21 (d, J=7.4 Hz, 1H), 7.55 (m, 1H), 7.39 (m, 1H), 6.88 (m, 1H), 6.18 (s, 1H), 5.59 (s, 1H), 5.55 (s, 1H), 4.22 (s, 1H), 3.81 (s, 3H), 2.13 (s, 3H), 1.48 (br s, 6H).

### Example 55

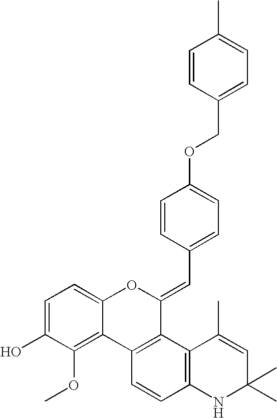

(Z)-5-(4′-(4″-methylbenzyloxy)benzylidene)-1,2-dihydro-9-hydroxy-10-methoxy-2,2,4-trimethyl-5H-chromeno[3,4-f]quinoline (Compound 70, structure 1 of Scheme I, where R¹=4-(4′-methylbenzyloxy)phenyl)

This compound was prepared according to General Method 1 (Example 1) from 4-(4′-methylbenzyloxy)benzyl bromide. ¹H NMR (500 MHz, CD₃OD) δ 8.26 (d, J=8.6 Hz, 1H), 7.66 (d, J=8.9 Hz, 2H), 7.32 (d, J=7.9 Hz, 2H), 7.19 (d, J=7.9 Hz, 2H), 6.97 (d, J=8.9 Hz, 2H), 6.80 (d, J=8.9 Hz, 1H), 6.73 (d, J=8.6 Hz, 1H), 6.71 (d, J=8.9 Hz, 1H), 5.50 (s, 2H), 5.04 (s, 2H), 3.75 (s, 3H), 2.34 (s, 3H), 2.05 (s, 3H), 1.30 (br s, 6H).

### Example 56

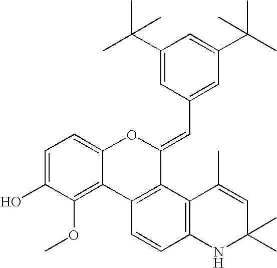

(Z)-5-(3′,5′-di-tert-butylbenzylidene)-1,2-dihydro-9-hydroxy-10-methoxy-2,2,4-trimethyl-5H-chromeno[3,4-f]quinoline (Compound 71, structure 1 of Scheme I, where R¹=3,5-di-tert-butylphenyl)

This compound was prepared according to General Method 1 (Example 1) from 3,5-di-tertbutylbenzyl bromide. ¹H NMR (500 MHz, CD₃OD) δ 8.27 (d, J=8.9 Hz, 1H), 7.59-7.57 (m, 2H), 7.32 (app t, J=1.2 Hz, 1H), 6.79-6.71 (m, 3H), 5.57 (s, 1H), 5.53 (d, J=1.2 Hz, 1H), 3.76 (s, 3H), 2.09 (d, J=1.0 Hz, 3H), 1.38-1.36 (m, 18H), 1.31 (br s, 6H).

### Example 57

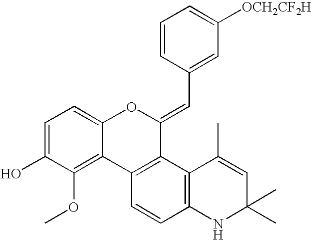

(Z)-5-(3′-(2″,2″-difluoroethoxy)benzylidene)-1,2-dihydro-9-hydroxy-10-methoxy-2,2,4-trimethyl-5H-chromeno[3,4-f]quinoline (Compound 72, structure 1 of Scheme I, where R¹=3-(2′,2′-difluoroethoxy)phenyl)

This compound was prepared according to General Method 1 (Example 1) from 3-(2′,2′-difluoroethoxy)benzyl bromide. ¹H NMR (500 MHz, CD₃OD) δ 8.27 (d, J=8.9 Hz, 1H), 7.42 (s, 1H), 7.28-7.25 (m, 2H), 6.87-6.71 (m, 4H), 6.19 (tt, J=50.1, 2.9 Hz, 1H), 5.54 (s, 1H), 5.49 (s, 1H), 4.23 (t, J=12.3 Hz, 2H), 3.74 (s, 3H), 2.04 (s, 3H), 1.29 (br s, 6H).

### Example 58

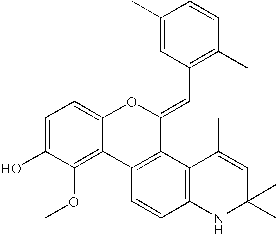

(Z)-5-(2′,5′-dimethylbenzylidene)-1,2-dihydro-9-hydroxy-10-methoxy-2,2,4-trimethyl-5H-chromeno[3,4-f]quinoline (Compound 73, structure 1 of Scheme I, where R¹=2,5-dimethylphenyl)

This compound was prepared according to General Method 1 (Example 1) from 2,5-dimethylbenzyl bromide. ¹H NMR (500 MHz, CD₃OD) δ 8.30 (d, J=8.6 Hz, 1H), 7.96 (s, 1H), 7.03 (d, J=7.6 Hz, 1H), 6.92 (d, J=7.3 Hz, 1H), 6.76 (d, J=8.6 Hz, 1H), 6.72 (m, 2H), 5.83 (s, 1H), 5.50 (s, 1H), 3.77 (s, 3H), 2.36 (s, 3H), 2.21 (s, 3H), 2.11 (s, 3H), 1.31 (br s, 6H).

### Example 59

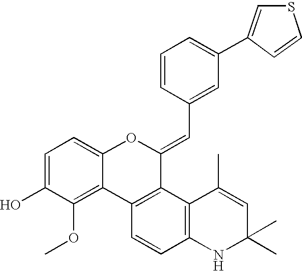

(Z)-5-(3′-(3″-thienyl)benzylidene)-1,2-dihydro-9-hydroxy-10-methoxy-2,2,4-trimethyl-5H-chromeno[3,4-f]quinoline (Compound 74, structure 1 of Scheme I, where R¹=3-(3′-thienyl)phenyl)

This compound was prepared according to General Method 1 (Example 1) from 3-(3′-thienyl)benzyl bromide. ¹H NMR (500 MHz, CD₃OD) δ 8.30 (d, J=8.9 Hz, 1H), 8.03 (s, 1M, 7.63-7.61 (m, 2H), 7.50-7.47 (m, 3H), 7.37 (t, J=7.6 Hz, 1H), 6.83 (d, J=8.9 Hz, 1H), 6.77 (d, J=8.9 Hz, 1H), 6.75 (d, J=9.8 Hz, 1H), 5.63 (s, 1H), 5.53 (s, 1H), 3.77 (s, 3H), 2.09 (s, 3H), 1.32 (br s, 6H).

### Example 60

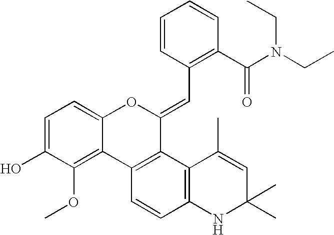

(Z)-5-(2′-diethylaminocarbonylbenzylidene)-1,2-dihydro-9-hydroxy-10-methoxy-2,2,4-trimethyl-5H-chromeno[3,4-f]quinoline (Compound 75, structure 1 of Scheme 1, where R¹=2-diethylaminocarbonylphenyl)

General Method 2: N,N-diethyl-o-toluamide (230 mg, 1.2 mmol) was dissolved in tetrahydrofuran (1 mL) and added to a stirring solution of lithium diisopropylamide (1.6 mmol) in tetrahydrofuran (5 mL) at −78° C. After 30 min, a solution of 9-hydroxy-10-methoxy-2,2,4-trimethyl-1,2-dihydro-5H-chromeno[3,4-f]quinoline-5-one (20 mg, 0.06 mmol) in tetrahydrofuran (1 mL) was added to a solution of the organolithium. The reaction was warmed to room temperature, then processed and carried forward as in the General Method 1. ¹H NMR (500 MHz, CD₃OD) δ 8.39-8.37 (m, 1H), 8.28 (d, J=8.3 Hz, 1H), 7.47-7.33 (m, 2H), 7.27-7.23 (m, 1H), 7.16-7.12 (m, 1H), 6.75-6.72 (m, 1H), 6.69-6.67 (m, 1H), 5.70 (s, 1H), 5.48-5.46 (m, 1H), 3.72 (s, 3H), 3.50-3.48 (m, 1H), 3.41-3.39 (m, 1H), 3.18-3.00 (m, 2H), 2.07 (m, 3H), 1.40-0.9 (m, 12H).

### Example 61

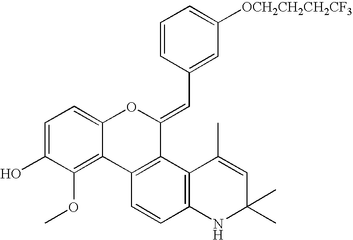

(Z)-5-(3′-(4″,4″,4″-trifluorobutoxy)benzylidene)-1,2-dihydro-9-hydroxy-10-methoxy-2,2,4-trimethyl-5H-chromeno[3,4-f]quinoline (Compound 76, structure 1 of Scheme I, where R¹=3-(4′,4′,4′-trifluorobutoxy)phenyl)

This compound was prepared according to General Method 1 (Example 1) from 3-(4′,4′,4′-trifluorobutoxy)benzyl bromide. ¹H NMR (500 MHz, CD₃OD) δ 8.28 (d, J=8.9 Hz, 1H), 7.39 (d, J=1.2 Hz, 1H), 7.24-7.20 (m, 2H), 6.81-6.71 (m, 4H), 5.54 (s, 1H), 5.51 (d, J=1.2 Hz, 1H), 4.09 (t, J=6.1 Hz, 2H), 3.76 (s, 3H), 2.42-2.38 (m, 2H), 2.07-2.05 (m, 4H), 1.31 (br s, 6H).

### Example 62

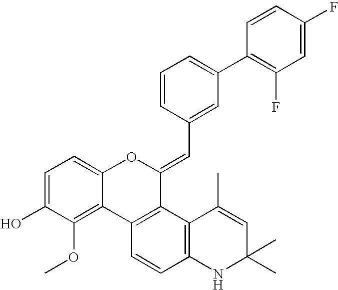

(Z)-5-(3′-(2″,4″-difluorophenyl)benzylidene)-1,2-dihydro-9-hydroxy-10-methoxy-2,2,4-trimethyl-5H-chromeno[3,4-f]quinoline (Compound 77, structure 1 of Scheme I, where R¹=3-(2′,4′-difluorophenyl)phenyl)

This compound was prepared according to General Method 1 (Example 1) from 3-(2′,4′-difluorophenyl)benzyl bromide. ¹H NMR (500 MHz, CD₃OD) δ 8.28 (d, J=8.9 Hz, 1H), 7.91 (s, 1H), 7.66 (d, J=7.6 Hz, 1H), 7.55-7.48 (m, 1H), 7.42 (t, J=7.6 Hz, 1H), 7.33 (d, J=7.6 Hz, 1H), 7.07-7.04 (m, 2H), 6.77 (d, J=8.9 Hz, 1H), 6.75 (d, J=8.6 Hz, 1H), 6.71 (d, J=8.9 Hz, 1H), 5.62 (s, 1H), 5.51 (s, 1H), 3.75 (s, 3H), 2.07 (s, 3H), 1.30 (br s, 6H).

### Example 63

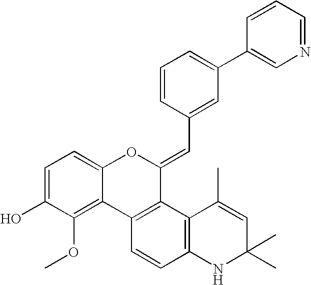

(Z)-5-(3′-(3″-pyridyl)benzylidene)-1,2-dihydro-9-hydroxy-10-methoxy-2,2,4-trimethyl-5H-chromeno[3,4-f]quinoline (Compound 78, structure 1 of Scheme I, where R¹=3-(3′-pyridyl)phenyl)

This compound was prepared according to General Method 1 (Example 1) from 3-(3′-pyridyl)benzyl bromide. ¹H NMR (500 MHz, CD₃OD) δ 8.82 (s, 1H), 8.57 (s, 1H), 8.30 (d, J=8.6 Hz, 1H), 8.16 (d, J=8.6 Hz, 1H), 8.05 (s, 1H), 7.77-7.74 (m, 1H), 7.61-7.56 (m, 1H), 7.48-7.45 (m, 2H), 6.81-6.73 (m, 3H), 5.67 (s, 1H), 5.53 (s, 1H), 3.76 (s, 3H), 2.09 (s, 3H), 1.32 (br s, 6H).

### Example 64

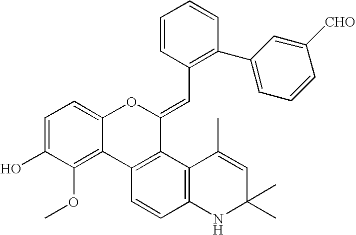

(Z)-5-(2′-(3″-formylphenyl)benzylidene)-1,2-dihydro-9-hydroxy-10-methoxy-2,2,4-trimethyl-5H-chromeno[3,4-f]quinoline (Compound 79, structure 1 of Scheme I, where R¹=2-(3′-formylphenyl)phenyl)

This compound was prepared according to General Method 1 (Example 1) from Compound 21 and 3-carbaldehydephenylboronic acid. ¹H NMR (500 MHz, CDCl₃) δ 9.98 (s, 1H), 8.25 (d, J=8.5 Hz, 1H), 7.87 (m, 1H), 7.77 (m, 1H), 7.55 (m, 2H), 7.48 (m, 1H), 7.29 (m, 1H), 7.25 (m, 1H), 6.82 (d, J=8.5 Hz, 1H), 6.73 (d, J=8.9 Hz, 1H). 6.67 (d, J=8.9 Hz, 1H), 5.51 (s, 1H), 5.17 (d, J=1.2 Hz, 1H), 3.81 (s, 3H), 1.91 (s, 3H), 1.29 (br s, 6H).

### Example 65

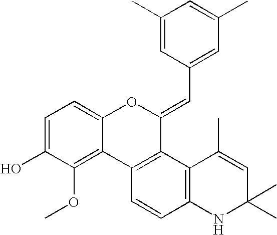

(Z)-5-(3′,5′-dimethylbenzylidene)-1,2-dihydro-9-hydroxy-10-methoxy-2,2,4-trimethyl-5H-chromeno[3,4-f]quinoline (Compound 80, structure 1 of Scheme I, where R¹=3,5-dimethylphenyl)

This compound was prepared according to General Method 1 (Example 1) from 3,5-dimethylbenzyl bromide. ¹H NMR (500 MHz, CD₃OD) δ 8.27 (d, J=8.9 Hz, 1H), 7.59-7.57 (m, 2H), 7.32 (app t, J=1.2 Hz, 1H), 6.79-6.71 (m, 3H), 5.57 (s, 1H), 5.53 (d, J=1.2 Hz, 1H), 3.76 (s, 3H), 2.28 (s, 3H), 2.18 (s, 3H), 2.09 (s, 3H), 1.31 (br s, 6H).

### Example 66

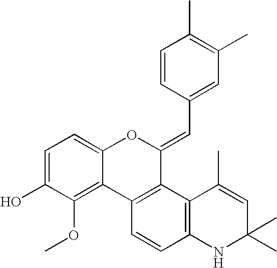

(Z)-5-(3′,4′-dimethylbenzylidene)-1,2-dihydro-9-hydroxy-10-methoxy-2,2,4-trimethyl-5H-chromeno[3,4-f]quinoline (Compound 81, structure 1 of Scheme I, where R¹=3,4-dimethylphenyl)

This compound was prepared according to General Method 1 (Example 1) from 3,4-dimethylbenzyl bromide. ¹H NMR (500 MHz, CD₃OD) δ 8.26 (d, J=8.9 Hz, 1H), 7.47 (d, J=1.5 Hz, 1H), 7.00 (dd, J=8.4, 1.4 Hz, 1H), 6.80-6.78 (m, 2H), 6.73-6.71 (m, 2H), 5.50-5.48 (m, 2H), 3.75 (s, 3H), 2.28 (s, 3H), 2.17 (s, 3H), 2.04 (s, 3H), 1.30 (br s, 6H).

### Example 67

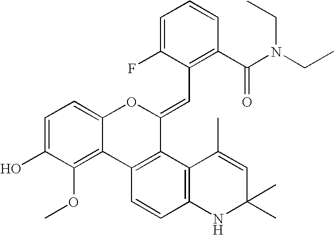

(Z)-5-(2′-(diethylamino)carbonyl-6′-fluorobenzylidene)-1,2-dihydro-9-hydroxy-10-methoxy-2,2,4-trimethyl-5H-chromeno[3,4-f]quinoline (Compound 82, structure 1 of Scheme I, where R¹=2-(diethylamino)carbonyl-6-fluorophenyl)

This compound was prepared according to General Method 2 (Example 60) from 6-fluoro-2-(diethylaminocarbonyl)toluene. ¹H NMR (500 MHz, CD₃OD) δ 8.30 (d, J=8.5 Hz, 1H), 7.40-7.35 (m, 2H), 7.25 (app t, J=8.9 Hz, 1H), 7.18 (m, 1H), 7.06 (d, J=7.6 Hz, 1H), 7.00 (dd, J=7.6, 1.0 Hz, 1H), 6.77 (d, J=8.5 Hz, 1H), 6.60 (d, J=8.9 Hz, 1H), 6.42 (d, J=8.5 Hz, 1H), 5.49 (s, 1H), 5.48 (d, J=1.2 Hz, 1H), 3.73 (s, 3H), 3.50 (br s, 1H), 3.05 (br s, 1H), 2.85 (br s, 1H), 2.62 (br s, 1H), 2.17 (d, J=1.2 Hz, 3H), 1.34-1.19 (m, 9H), 1.08 (t, J=7.2 Hz, 3H).

### Example 68

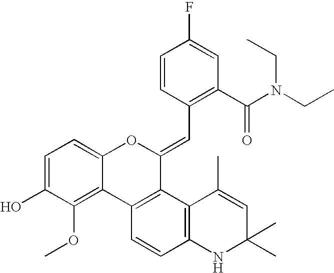

(Z)-5-(2′-(diethylamino)carbonyl-4′-fluorobenzylidene)-1,2-dihydro-9-hydroxy-10-methoxy-2,2,4-trimethyl-5H-chromeno[3,4-f]quinoline (Compound 83, structure 1 of Scheme I, where R¹=2-(diethylamino)carbonyl-4-fluorophenyl)

This compound was prepared according to General Method 2 (Example 60) from 4-fluoro-2-(diethylaminocarbonyl)toluene. ¹H NMR (500 MHz, CD₃OD) δ 8.41-8.39 (m, 1H), 8.26 (d, J=8.5 Hz, 1H), 7.22 (ddd, J=11.6, 8.9, 2.9 Hz, 1H), 6.95 (dd, J=8.5, 3.1 Hz, 1H), 6.75-6.68 (m, 3H), 5.51 (1H, s), 5.44 (d, J=1.2 Hz, 1H), 3.72 (s, 3H), 3.44 (br s, 1H), 3.40 (br s, 1H), 3.05 (br s, 2H), 2.03 (d, J=1.2 Hz, 3H), 1.28-1.19 (m, 12H).

### Example 69

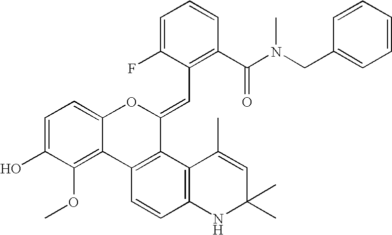

(Z)-5-(2′-(methylbenzylamino)carbonyl-6′-fluorobenzylidene)-1,2-dihydro-9-hydroxy-10-methoxy-2,2,4-trimethyl-5H-chromeno[3,4-f]quinoline (Compound 84, structure 1 of Scheme I, where R¹=2-(methylbenzylamino)carbonyl-6-fluorophenyl)

This compound was prepared according to General Method 2 (Example 60) from 6-fluoro-2-(N-methyl-N-benzylaminocarbonyl)toluene. ¹H NMR (500 MHz, CD₃OD) δ 8.36 (d, J=8.9 Hz, 0.3H), 8.24 (d, J=8.9 Hz, 0.7H), 7.44-7.39 (m, 1H), 7.30-7.15 (m, 1H), 7.13-7.02 (m, 2H), 6.94-6.80 (m, 6H), 6.59 (d, J=8.9 Hz, 0.3H), 6.51 (d, J=8.9 Hz, 0.7H), 6.45 (d, J=8.9 Hz, 0.3H), 6.36 (d, J=8.9 Hz, 0.7H), 5.67 (s, 0.7H) 5.60 (s, 0.3H), 5.51 (d, J=1.2 Hz, 1H), 5.22-5.20 (m, 2H), 3.80 (s, 3H), 2.21 (m, 3H), 1.48-1.10 (m, 6H).

### Example 70

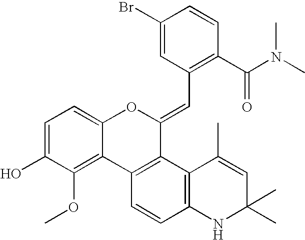

(Z)-5-(2′-(dimethylamino)carbonyl-5′-bromo-benzylidene)-1,2-dihydro-9-hydroxy-10-methoxy-2,2,4-trimethyl-5H-chromeno[3,4-f]quinoline (Compound 85, structure 1 of Scheme I, where R¹=2-(dimethylamino)carbonyl-5-bromophenyl)

This compound was prepared according to General Method 2 (Example 60) from 5-bromo-2-(dimethylaminocarbonyl)toluamide. ¹H NMR (500 MHz, CD₃OD) δ 8.63 (d, J=1.8 Hz, 1H), 8.34 (d, J=8.9 Hz, 1H), 7.42 (dd, J=8.1, 2.0 Hz, 1H), 7.11 (d, J=8.2 Hz, 1H), 6.79-6.75 (m, 3H), 5.51 (d, J=1.2 Hz, 1H), 5.49 (s, 1H), 3.77 (s, 3H), 3.03 (s, 3H), 2.79 (br s, 3H), 2.03 (d, J=1.2 Hz, 3H), 1.29 (br s, 6H).

### Example 71

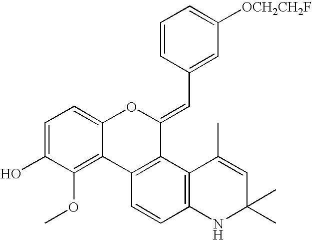

(Z)-5-(3′-(2″-fluoroethoxy)benzylidene)-1,2-dihydro-9-hydroxy-10-methoxy-2,2,4-trimethyl-5H-chromeno[3,4-f]quinoline (Compound 86, structure 1 of Scheme I, where R¹=3-(2′-fluoroethoxy)phenyl)

This compound was prepared according to General Method 1 (Example 1) from 3-(2′-fluoroethoxy)benzyl bromide. ¹H NMR (500 MHz, CD₃OD) δ 8.27 (d, J=8.9 Hz, 1H), 7.42 (s, 1H), 7.28-7.25 (m, 2H), 6.87-6.71 (m, 4H), 6.19 (tt, J=50.1, 2.9 Hz, 1H), 5.54 (s, 1H), 5.49 (s, 1H), 4.23 (t, J=12.3 Hz, 2H), 3.74 (s, 3H), 2.04 (s, 3H), 1.29 (br s, 6H).

### Example 72

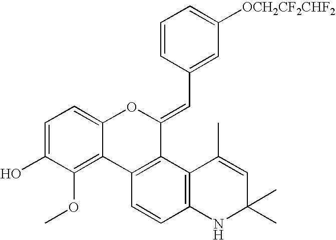

(Z)-5-(3′-(2″,2″,3″,3″-tetrafluoropropoxy)benzylidene)-1,2-dihydro-9-hydroxy-10-methoxy-2,2,4-trimethyl-5H-chromeno[3,4-f]quinoline (Compound 87, structure 1 of Scheme I, where R¹=3-(2′,2′,3′,3′-tetrafluoropropoxy)phenyl)

This compound was prepared according to General Method 1 (Example 1) from 3-(2′,2′,3′,3′-tetrafluoropropoxy)benzyl bromide. ¹H NMR (500 MHz, CD₃OD δ 8.28 (d, J=8.6 Hz, 1H), 7.41 (s, 1H), 7.35 (d, J=7.6 Hz, 1H), 7.29 (t, J=7.6 Hz, 1H), 6.86 (d, J=8.2 Hz, 1H), 6.80 (d, J=8.9 Hz, 1H), 6.75 (d, J=8.9 Hz, 1H), 6.72 (d, J=8.9 Hz, 1H), 6.35 (tt, J=50.1, 3.1 Hz, 1H), 5.55 (s, 1H), 5.51 (s, 1H), 4.47 (t, J=12.5 Hz, 2H), 3.75 (s, 3H), 2.05 (s, 3H), 1.30 (br s, 6H).

### Example 73

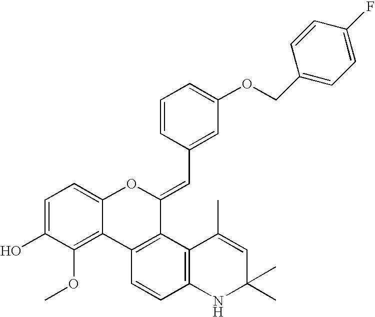

(Z)-5-(3′-(4″-fluorobenzyloxy)benzylidene)-1,2-dihydro-9-hydroxy-10-methoxy-2,2,4-trimethyl-5H-chromeno[3,4-f]quinoline (Compound 88, structure 1 of Scheme I, where R¹=3-(4′-fluorobenzyloxy)phenyl)

This compound was prepared according to General Method 1 (Example 1) from 3-(4′-fluorobenzyloxy)benzyl bromide. ¹H NMR (500 MHz, CD₃OD) δ 8.27 (d, J=8.6 Hz, 1H), 7.50-7.42 (m, 3H), 7.28-7.19 (m, 2H), 7.11 (t, J=8.9 Hz, 2H), 6.82 (d, J=8.6 Hz, 1H), 6.75 (d, J=8.6 Hz, 1H), 6.70 (s, 2H), 5.54 (s, 1H), 5.51 (s, 1H), 5.11 (s, 2H), 3.75 (s, 3H), 2.05 (s, 3H), 1.30 (br s, 6H).

### Example 74

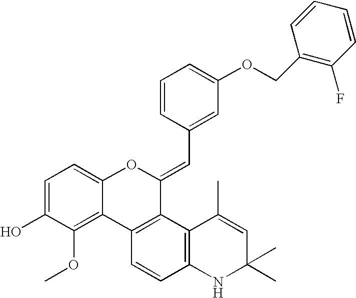

(Z)-5-(3′-(2″-fluorobenzyloxy)benzylidene)-1,2-dihydro-9-hydroxy-10-methoxy-2,2,4-trimethyl-5H-chromeno[3,4-f]quinoline (Compound 89, structure 1 of Scheme I, where R¹=3-(2′-fluorobenzyloxy)phenyl)

This compound was prepared according to General Method 1 (Example 1) from 3-(2′-fluorobenzyloxy)benzyl bromide. ¹H NMR (500 MHz, CD₃OD) δ 8.27 (d, J=8.6 Hz, 1H), 7.56-7.49 (m, 2H), 7.38-7.31 (m, 1H), 7.24-7.12 (m, 4H), 6.83 (d, J=8.5 Hz, 1H), 6.74 (d, J=8.9 Hz, 1H), 6.72-6.69 (m, 2-overlapping signals, 2H), 5.54 (s, 1H), 5.50 (s, 1H), 5.18 (s, 2H), 3.75 (s, 3H), 2.05 (s, 3H), 1.30 (br s, 6H).

### Example 75

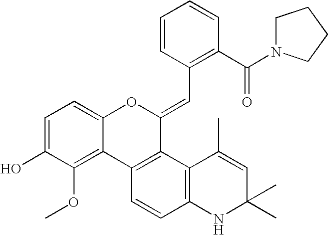

(Z)-5-(2′-(pyrrolidinecarbonyl)benzylidene)-1,2-dihydro-9-hydroxy-10-methoxy-2,2,4-trimethyl-5H-chromeno[3,4-f]quinoline (Compound 90, structure 1 of Scheme I, where R¹=2-(pyrrolidine)carbonylphenyl)

This compound was prepared according to General Method 2 (Example 60) from 2-(pyrrolidinecarbonyl)toluene. ¹H NMR (500 MHz, CD₃OD) δ 8.42 (d, J=7.9 Hz, 1H), 8.30 (d, J=8.9 Hz, 1H), 7.47 (ddd, J=9.0, 7.9, 1.5 Hz, 1H), 7.27 (ddd, J=8.5, 7.3, 0.9 Hz, 1H), 7.20 (dd, J=7.6, 1.5 Hz, 1H), 6.80-6.71 (m, 3H), 5.58 (s, 1H), 5.47 (d, J=1.2 Hz, 1H), 3.74 (s, 3H), 3.50 (m, 2H), 3.10 (m, 2H), 2.05 (d, J=1.2 Hz, 3), 1.90 (m, 2H), 1.78 (m, 2H), 1.29 (br s, 6H).

### Example 76

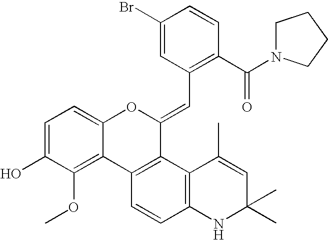

(Z)-5-(2′-(pyrrolidinecarbonyl)-5′-bromobenzylidene)-1,2-dihydro-9-hydroxy-10-methoxy-2,2,4-trimethyl-5H-chromeno[3,4-f]quinoline (Compound 91, structure 1 of Scheme I, where R¹=5-bromo-2-pyrrolidine)carbonylphenyl)

This compound was prepared according to General Method 2 (Example 60) from 5-bromo-2-pyrrolidinecarbonyl)toluene. ¹H NMR (500 MHz, CD₃OD) δ 8.63 (d, J=1.8 Hz, 1H), 8.34 (d, J=8.9 Hz, 1H), 7.25 (dd, J=8.0, 2.0 Hz, 1H), 7.25 (dd, J=8.0, 2.0 Hz, 1H), 7.15 (d, J=8.2 Hz, 1H), 6.79-6.74 (m, 3H), 5.54 (s, 1H), 5.48 (d, J=1.2 Hz, 1H), 3.76 (s, 3H), 3.50 (m, 2H), 3.12 (m, 2H), 2.04 (d, J=1.2 Hz, 3H), 1.91 (m, 2H), 1.82 (m, 2H), 1.28 (br s, 6H).

### Example 77

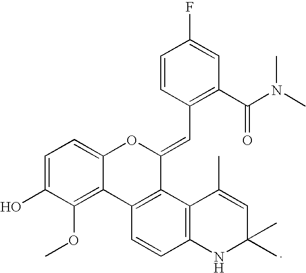

(Z)-5-(2′-(dimethylaminocarbonyl)-4′-fluorobenzylidene)-1,2-dihydro-9-hydroxy 10-methoxy-2,2,4-trimethyl-5H-chromeno[3,4-f]quinoline (Compound 92, structure 1 of Scheme I, where R¹=4-fluoro-2-(N,N-dimethylaminocarbonyl)phenyl)

This compound was prepared according to General Method 2 (Example 60) from 4-fluoro-2-(dimethylaminocarbonyl)toluene. ¹H NMR (500 MHz, CD₃OD) δ 8.47 (dd, J=8.9, 5.5 Hz, 1H), 8.46 (d, J=8.9 Hz, 1H), 7.23 (ddd, J=11.4, 8.7, 2.8 Hz, 1H), 6.95 (dd, J=8.5, 2.7 Hz, 1H), 6.81 (d, J=8.5 Hz, 1H), 6.76 (d, J=8.5 Hz, 1H), 6.72 (d, J=8.9 Hz, 1H), 5.49 (d, J=1.2 Hz, 1H), 5.48 (1H, s), 3.79 (s, 3H), 3.03 (s, 3H), 2.80 (br s, 3H), 2.03 (d, J=1.2 Hz, 3H), 1.29 (m, 6H).

### Example 78

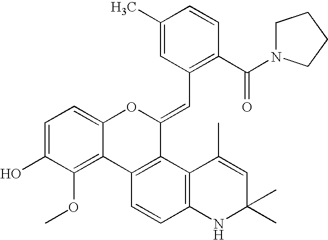

(Z)-5-(2′-(pyrrolidinecarbonyl)-5′-methylbenzylidene)-1,2-dihydro-9-hydroxy-10-methoxy-2,2,4-trimethyl-5H-chromeno[3,4-f]quinoline (Compound 93, structure 1 of Scheme I, where R¹=5-methyl-2-(pyrrolidine)carbonylphenyl)

This compound was prepared according to General Method 2 (Example 60) from 5-methyl-2-(pyrrolidinecarbonyl)toluene. ¹H NMR (500 MHz, CD₃OD) δ 8.30 (d, J=8.9 Hz, 1H), 8.25 (s, 1H), 7.11 (d, J=0.9 Hz, 1H), 6.79-6.72 (m, 3H), 5.55 (s, 1H), 5.47 (d, J=1.2 Hz, 1H), 3.75 (s, 3H), 3.49 (m, 2H), 3.10 (m, 2H), 2.44 (s, 3H), 2.05 (d, J=1.2 Hz, 3H), 1.90 (m, 2H), 1.80 (m, 2H), 1.28 (br s, 6H).

### Example 79

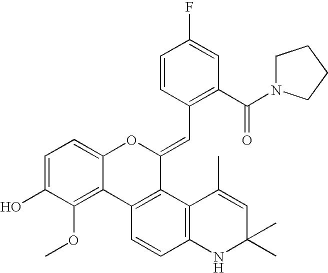

(Z)-5-(2′-(pyrrolidinecarbonyl)-4′-fluorobenzylidene)-1,2-dihydro-9-hydroxy-10-methoxy-2,2,4-trimethyl-5H-chromeno[3,4-f]quinoline (Compound 94, structure 1 of Scheme I, where R¹=4-fluoro-2-(pyrrolidine)carbonylphenyl)

This compound was prepared according to General Method 2 (Example 60) from 4-fluoro-2-(pyrrolidinecarbonyl)toluene. ¹H NMR (500 MHz, CD₃OD) δ 8.45 (dd, J=8.9, 5.5 Hz, 1H), 8.31-8.29 (m, 1H), 7.23 (ddd, J=11.6, 8.9, 3.1 Hz, 1H), 7.02 (dd, J=8.5, 3.1 Hz, 1H), 6.82-6.70 (m, 3H), 5.52 (1H, s), 5.48 (d, J=1.2 Hz, 1H), 3.75 (s, 3H), 3.50 (m, 2H), 3.12 (m, 2H), 2.04 (d, J=1.2 Hz, 3H), 1.89 (m, 2H), 1.80 (m, 2H), 1.28 (br s, 6H).

### Example 80

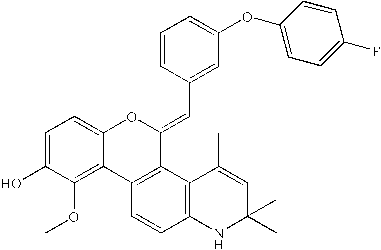

(Z)-5-(3′-(4″-fluorophenoxy)benzylidene)-1,2-dihydro-9-hydroxy-10-methoxy-2,2,4-trimethyl-5H-chromeno[3,4-f]quinoline (Compound 95, structure 1 of Scheme I, where R¹=3-(4′-fluorophenoxy)phenyl)

This compound was prepared according to General Method 1 (Example 1) from 3-(4′-fluorophenoxy)benzyl bromide. ¹H NMR (400 MHz, CD₃OD) δ 8.24 (d, J=8.6 Hz, 1H), 7.56 (s, 1H), 7.28 (t, J=7.9 Hz, 1H), 7.18-7.07 (m, 5H), 6.86 (d, J=7.9 Hz, 1H), 6.72 (d, J=8.9 Hz, 1H), 6.60 (d, J=8.9 Hz, 1H), 6.22 (d, J=8.8 Hz, 1H), 5.50 (s, 1H), 5.48 (s, 1H), 3.72 (s, 3H), 2.02 (s, 3H), 1.28 (br s, 6H).

### Example 81

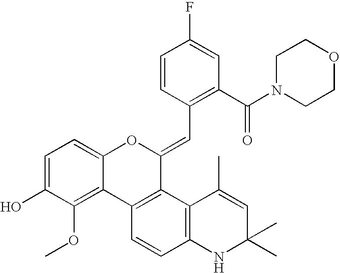

(Z)-5-(2′-(morpholinecarbonyl)-4′-fluorobenzylidene)-1,2-dihydro-9-hydroxy-10-methoxy-2,2,4-trimethyl-5H-chromeno[3,4-f]quinoline (Compound 96, structure 1 of Scheme I, where R¹=4-fluoro-2-(morpholinecarbonyl)phenyl)

This compound was prepared according to General Method 2 (Example 60) from 4-fluoro-2-(morpholinecarbonyl)toluene. ¹H NMR (500 MHz, CD₃OD) δ 8.40-8.38 (m, 1H), 8.30 (d, J=8.5 Hz, 1H), 7.25 (ddd, J=11.4, 8.7, 2.7 Hz, 1H), 7.03 (dd, J=8.5, 3.1 Hz, 1H), 6.79-6.74 (m, 1H), 6.71 (d, J=8.9 Hz, 1H), 5.55 (1H, s), 5.50 (d, J=1.2 Hz, 1H), 3.74 (s, 3H), 3.67-3.65 (m, 2H), 3.59-3.56 (m, 2H), 3.46-3.44 (m, 1H), 3.23-3.21 (m, 1H), 3.11-3.09 (m, 1H), 2.05 (d, J=1.2 Hz, 3H), 1.32 (s, 3H), 1.28 (s, 3H).

### Example 82

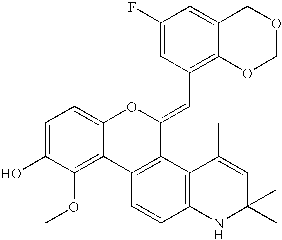

(Z)-5-(8′-(6′-fluoro-benzo-1′,3′-dioxan-methylidiene))-1,2-dihydro-9-hydroxy-10-methoxy-2,2,4-trimethyl-5H-chromeno[3,4-f]quinoline (Compound 97, structure 1 of Scheme I, where R¹=8-(6-fluoro-benzo-1,3-dioxan))

This compound was prepared according to General Method 1 (Example 1) from 8-chloromethyl-6-fluoro-benzo-1,3-dioxane. ¹H NMR (500 MHz, CDCl₃) δ 8.15 (d, J=8.9 Hz, 1H), 7.87 (dd, J=10.7, 2.8 Hz, 1H), 6.83 (dd, J=9.0, 8.2 Hz, 2H), 6.66 (d, J=8.6 Hz, 1H), 6.54 (dd, J=8.2, 3.1 Hz, 1H), 5.97 (s, 1H), 5.55 (s, 1H), 5.49 (s, 1H), 5.19 (s, 2H), 4.85 (s, 2H), 4.17 (s, 1H), 3.75 (s, 3H), 2.08 (s, 3H), 1.33 (br s, 6H).

### Example 83

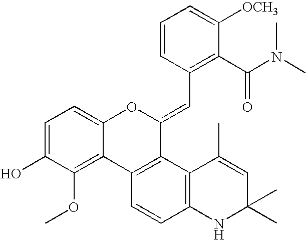

(Z)-5-(2′-dimethylaminocarbonyl-3′-methoxybenzylidene)-1,2-dihydro-9-hydroxy-10-methoxy-2,2,4-trimethyl-5H-chromeno[3,4-f]quinoline (Compound 98, structure 1 of Scheme I, where R¹=2-(dimethylcarbonyl)-3-methoxyphenyl)

This compound was prepared according to General Method 2 (Example 60) from 3-methoxy-2-(N,N-dimethylaminocarbonyl)toluene. ¹H NMR (500 MHz, CD₃OD) δ 8.30 (d, J=8.5 Hz, 1H), 8.07 (d, J=7.9 Hz, 1H), 7.42 (t, J=8.2 Hz, 1H), 6.92 (d, J=8.5 Hz, 1H), 6.81 (d, J=8.9 Hz, 1H), 6.76 (d, J=8.5 Hz, 1H), 6.73 (d, J=8.9 Hz, 1H), 5.49 (d, J=1.2 Hz, 1H), 5.47 (s, 1H), 3.82 (s, 3H), 3.75 (s, 3H), 3.04 (s, 3H), 2.80 (br s, 3H), 2.04 (s, 3H), 1.29 (s, 6H).

### Example 84

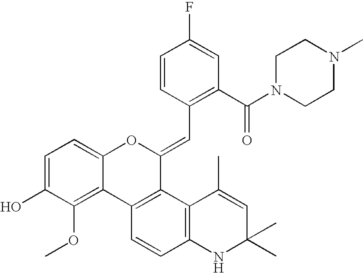

(Z)-5-(2′-(4″-methylpiperazinecarbonyl)-4′-fluorobenzylidene)-1,2-dihydro-9-hydroxy-10-methoxy-2,2,4-trimethyl-5H-chromeno[3,4-f]quinoline (Compound 99, structure 1 of Scheme I, where R¹=4-fluoro-2-(4′-methylpiperazine)carbonylphenyl)

This compound was prepared according to General Method 2 (Example 60) from 4-fluoro-2-(4′-methylpiperazinecarbonyl)toluene. ¹H NMR (500 MHz, CD₃OD) δ 8.40-8.38 (m, 1H), 8.31 (d, J=8.9 Hz, 1H), 7.25 (ddd, J=11.6, 8.9, 2.9 Hz, 1H), 7.02 (dd, J=8.5, 2.7 Hz, 1H), 6.76 (dd, J=8.9, 3.1 Hz, 1H), 6.70 (d, J=8.7 Hz, 1H), 5.51 (s, 1H), 5.50 (m, 1H) 3.75 (s, 3H), 3.63-3.61 (m, 1H), 3.30 (m, 2H, obscured by solvent), 3.15-3.05 (m, 2H), 2.43-2.41 (m, 2H), 2.21-2.19 (m, 4H), 2.04 (d, J=1.5 Hz, 3H), 1.31-1.28 (m, 6H).

### Example 85

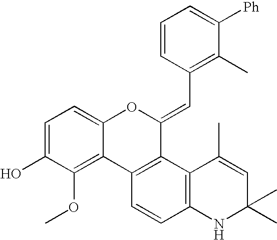

(Z)-5-(2′-methyl-3′-phenylbenzylidene)-1,2-dihydro-9-hydroxy-10-methoxy-2,2,4-trimethyl-5H-chromeno[3,4-f]quinoline (Compound 100, structure 1 of Scheme I, where R¹=2-methyl-3-phenylphenyl)

This compound was prepared according to General Method 1 (Example 1) from 2-methyl-3-phenylbenzyl bromide. ¹H NMR (500 MHz, CDCl₃) δ 8.17 (d, J=8.6 Hz, 1H), 8.10 (d, J=7.8 Hz, 1H), 7.38 (m, 2H), 7.33-7.29 (m, 4H), 7.10 (m, 1H), 6.81 (m, 2H), 6.68 (m, 1H), 5.91 (s, 1H), 5.54 (s, 1H), 5.49 (s, 1H), 4.18 (s, 1H), 3.80 (s, 3H), 2.18 (s, 3H), 2.15 (s, 3H), 1.35 (br s, 6H).

### Example 86

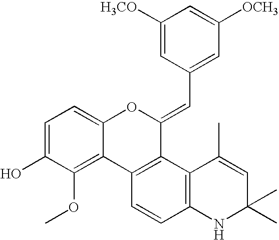

(Z)-5-(3′,5′-dimethoxybenzylidene)-1,2-dihydro-9-hydroxy-10-methoxy-2,2,4-trimethyl-5H-chromeno[3,4-f]quinoline (Compound 101, structure 1 of Scheme I, where R¹=3,5-dimethoxyphenyl)

This compound was prepared according to General Method 1 (Example 1) from 3,5-dimethoxybenzyl bromide. ¹H NMR (500 MHz, CDCl₃) δ 8.15 (d, J=8.9 Hz, 1H), 6.97 (s, 1H), 6.86 (d, J=8.6 Hz, 1H), 6.81 (d, J=8.6 Hz, 1H), 6.67 (d, J=8.7 Hz, 1H), 6.37 (s, 1H), 5.59 (s, 1H), 5.55 (s, 1H), 5.51 (s, 1H), 3.85 (s, 6H), 3.78 (s, 3H), 2.10 (s, 3H), 1.35 (br s, 6H).

### Example 87

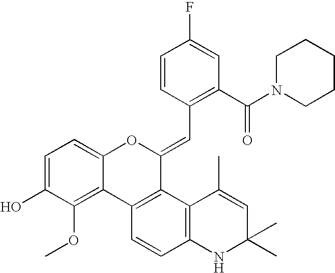

(Z)-5-(2′-piperidinecarbonyl)-4′-fluorobenzylidene)-1,2-dihydro-9-hydroxy-10-methoxy-2,2,4-trimethyl-5H-chromeno[3,4-f]quinoline (Compound 102, structure 1 of Scheme T, where R¹=4-fluoro-2-(piperidinecarbonyl)phenyl)

This compound was prepared according to General Method 2 (Example 60) from 4-fluoro-2-(piperidinecarbonyl)toluene. ¹H NMR (500 MHz, CD₃OD) δ 8.41-8.39 (m, 1H), 8.29 (d, J=8.5 Hz, 1H), 7.25 (ddd, J=11.6, 8.7, 2.7 Hz, 1H), 6.97 (dd, J=8.5, 2.7 Hz, 1H), 6.75 (d, J=8.9 Hz, 1H), 6.69 (d, J=8.5 Hz, 1H), 5.52 (1H, s), 5.47 (d, J=0.6 Hz, 1H), 3.73 (s, 3H), 3.69-3.67 (m, 1H), 3.53-3.52 (m, 1H), 3.14-3.08 (m, 2H), 2.04 (s, 3H), 1.57-1.54 (m, 4H) 1.30-1.28 (m, 8H).

### Example 88

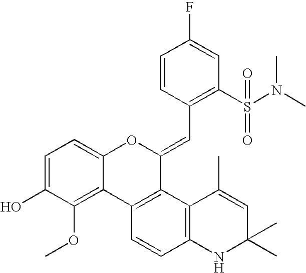

(Z)-5-(2′-dimethylaminosulphonyl-4′-fluorobenzylidene)-1,2-dihydro-9-hydroxy-10-methoxy-2,2,4-trimethyl-5H-chromeno[3,4-f]quinoline (Compound 103, structure 1 of Scheme I, where R¹=4-fluoro-2-(dimethylaminosulphonyl)phenyl)

This compound was prepared according to General Method 2 (Example 60) from 4-fluoro-2-(N,N-dimethylaminosulfonyl)toluene. ¹H NMR (500 MHz, CD₃OD) δ 8.36 (dd, J=8.9, 5.5 Hz, 1H), 8.29 (d, J=8.5 Hz, 1H), 7.63 (dd, J=8.9, 2.7 Hz, 1H), 7.46 (ddd, J=11.1, 8.4, 2.9 Hz, 1H), 6.78 (d, J=8.5 Hz, 1H), 6.63 (d, J=8.9 Hz, 1H), 6.62 (d, J=8.9 Hz, 1H), 6.43 (s, 1H), 5.49 (d, J=1.5 Hz, 1H), 3.73 (s, 3H), 2.48 (s, 6H), 2.08 (d, J=1.2 Hz, 3H), 1.30 (br s, 6H).

### Example 89

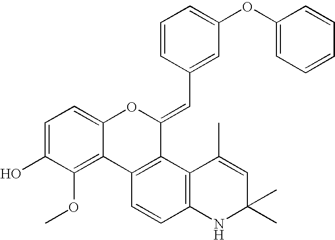

(Z)-5-(3′-phenoxybenzylidene)-1,2-dihydro-9-hydroxy-10-methoxy-2,2,4-trimethyl-5H-chromeno[3,4-f]quinoline (Compound 104, structure 1 of Scheme 1, where R¹=3-phenoxyphenyl)

This compound was prepared according to General Method 1 (Example 1) from 3-phenoxybenzyl bromide. ¹H NMR (500 MHz, CD₃OD) δ 8.25 (d, J=8.9 Hz, 1H), 7.60 (s, 1H), 7.42 (t, J=1.2 Hz, 2H), 7.29 (t, J=1.5 Hz, 1H), 7.20 (t, J=1.2 Hz, 1H), 7.16 (d, J=7.6 Hz, 1H), 7.08-7.06 (m, 2-overlapping signals, 2H), 6.87 (d, J=7.6 Hz, 1H), 6.73 (d, J=8.6 Hz, 1H), 6.59 (d, J=8.6 Hz, 1H), 6.22 (d, J=8.9 Hz, 1H), 5.52 (s, 1H), 5.50 (s, 1H), 3.73 (s, 3H), 2.03 (s, 3H), 1.29 (br s, 6H).

### Example 90

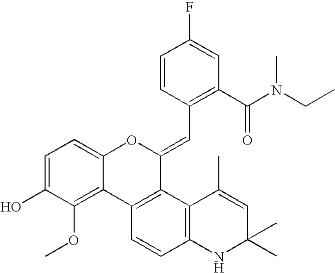

(Z)-5-(2′-(ethylmethylamino)carbonyl-4′-fluorobenzylidene)-1,2-dihydro-9-hydroxy-10-methoxy-2,2,4-trimethyl-5H-chromeno[3,4-f]quinoline (Compound 105, structure 1 of Scheme I, where R¹=4-fluoro-2-(ethylmethylamino)carbonylphenyl)

This compound was prepared according to General Method 2 (Example 60) from 4-fluoro-2-(ethylmethylaminocarbonyl)toluene. ¹H NMR (500 MHz, CD₃OD) δ 8.46 (dd, J=8.9, 5.6 Hz, 1H), 8.30 (m, 1H, rotamers), 7.23 (ddd, J=11.4, 8.7, 2.7 Hz, 1H), 6.99-6.95 (m, 1H, rotamers), 6.81-6.70 (m, 3H, rotamers), 5.52-5.47 (m, 2H, rotamers), 3.75-3.73 (m, 3H), 3.13-3.11 (m, 2H), 2.99 (s, 2H, rotamers), 2.72 (1H, s, rotamers), 2.04-2.02 (m, 3H), 1.27 (br s, 6H), 1.18-1.14 (m, 3H).

### Example 91

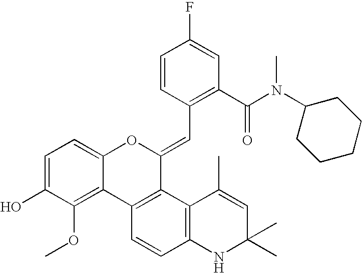

(Z)-5-(2′-(cyclohexylmethylamino)carbonyl-4′-fluorobenzylidene)-1,2-dihydro-9-hydroxy-10-methoxy-2,2,4-trimethyl-5H-chromeno[3,4-f]quinoline (Compound 106, structure 1 of Scheme I, where R¹=4-fluoro-2-(cyclohexylmethylamino)carbonylphenyl)

This compound was prepared according to General Method 2 (Example 60) from 4-fluoro-2-(N-cyclohexyl-N-methylaminocarbonyl)toluene. ¹H NMR (500 MHz, CD₃OD) δ 8.37-8.34 (m, 1H), 8.30-8.28 (m, 1H), 7.26-7.24 (m, 1H), 6.97-6.95 (m, 1H), 6.77-6.75 (m, 1H), 6.70-6.68 (m, 1H), 5.52-5.42 (m, 2H), 4.31-4.29 (m, 1H), 3.74-3.73 (m, 3H), 3.08-3.06 (m, 1H), 2.85-2.83 (m, 2H), 2.61-2.59 (m, 1H), 2.06-2.03 (m, 3H), 1.66-1.00 (m, 15H).

### Example 92

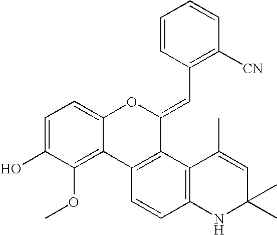

(Z)-5-(2′-cyanobenzylidene)-1,2-dihydro-9-hydroxy-10-methoxy-2,2,4-trimethyl-5H-chromeno[3,4-f]quinoline (Compound 107, structure 1 of Scheme I, where R¹=2-cyanophenyl)

This compound was prepared according to General Method 2 (Example 60) from 2-methyl-benzonitrile. ¹H NMR (500 MHz, CDCl₃) δ 8.45 (d, J=8.5 Hz, 1H), 8.20 (d, J=8.5 Hz, 1H), 7.60 (m, 2H), 7.25 (m, 1H), 6.85 (d, J=8.6 Hz, 1H), 6.74 (d, J=8.6 Hz, 1H), 6.13 (s, 1H), 5.60 (m, 2H), 3.81 (s, 3H), 2.12 (s, 3H), 1.37 (br s, 6H).

### Example 93

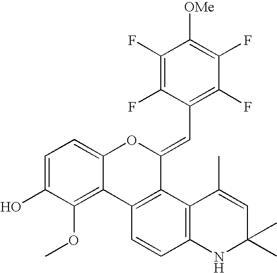

(Z)-5-(2′,3′,5′,6′-tetrafluoro-4′-methoxybenzylidene)-1,2-dihydro-9-hydroxy-10-methoxy-2,2,4-trimethyl-5H-chromeno[3,4-f]quinoline (Compound 108, structure 1 of Scheme I, where R¹=2,3,5,6-tetrafluoro-4-methoxyphenyl)

This compound was prepared according to General Method 1 (Example 1) from 2,3,5,6-tetrafluoro-4-methoxybenzyl bromide. ¹H NMR (500 MHz, CD₃OD) δ 8.35 (d, J=8.6 Hz, 1H), 6.80 (d, J=8.6 Hz, 1H), 6.67 (d, J=8.9 Hz, 1H), 6.57 (d, J=8.9 Hz, 1H), 5.51 (s, 1H), 5.42 (s, 1H), 4.07 (s, 3H), 3.77 (s, 3H), 2.14 (s, 3H), 1.29 (br s, 6H).

### Example 94

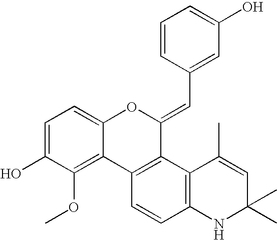

(Z)-5-(3′-hydroxybenzylidene)-1,2-dihydro-9-hydroxy-10-methoxy-2,2,4-trimethyl-5H-chromeno[3,4-f]quinoline (Compound 109, structure 1 of Scheme I, where R¹=3-hydroxyphenyl)

This compound was prepared according to General Method 1 (Example 1) from 3-trimethylsiloxybenzyl bromide. ¹H NMR (500 MHz, CD₃OD) δ 8.26 (d, J=8.9 Hz, 1H), 7.33-7.32 (m, 1H), 7.12 (t, J=7.9 Hz, 1H), 7.07-7.04 (m, 1H), 6.88 (d, J=8.6 Hz, 1H), 6.73 (d, J=8.6 Hz, 1H), 6.71 (d, J=8.9 Hz, 1H), 6.62 (d, J=8.6 Hz, 1H), 5.50 (s, 1H), 5.48 (s, 1H), 3.75 (s, 3H), 2.05 (s, 3H), 1.30 (br s, 6H).

### Example 95

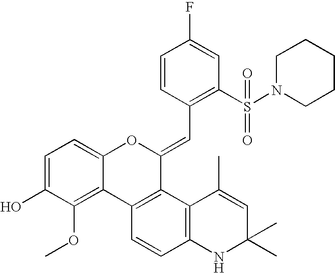

(Z)-5-(2′-(piperidinesulphonyl)-4′-fluorobenzylidene)-1,2-dihydro-9-hydroxy-10-methoxy-2,2,4-trimethyl-5H-chromeno[3,4-f]quinoline (Compound 110, structure 1 of Scheme I, where R¹=4-fluoro-2-(piperidinesulphonyl)phenyl)

This compound was prepared according to General Method 2 (Example 60) from 4-fluoro-2-(piperidinesulfonyl)toluene. ¹H NMR (500 MHz, CD₃OD) δ 8.30 (d, J=8.5 Hz, 1H), 8.26 (dd, J=8.9, 5.5 Hz, 1H), 7.64 (dd, J=8.9, 3.1 Hz, 1H), 7.46 (ddd, J=11.3, 8.2, 3.1 Hz, 1H), 6.79 (d, J=8.9 Hz, 1H), 6.64 (d, J=8.9 Hz, 1H), 6.59 (d, J=8.9 Hz, 1H), 6.35 (s, 1H), 5.50 (d, J=1.2 Hz, 1H), 3.73 (s, 3H), 2.82-2.80 (m, 4H), 2.09 (d, J=1.2 Hz, 3H), 1.30 (br s, 6H), 1.28-1.17 (m, 6H).

### Example 96

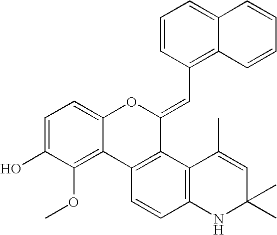

(Z)-5-(1′-naphthylmethylidene)-1,2-dihydro-9-hydroxy-10-methoxy-2,2,4-trimethyl-5H-chromeno[3,4-f]quinoline (Compound III, structure 1 of Scheme I, where R¹=1-naphthyl)

This compound was prepared according to General Method 1 (Example 1) from 1-bromomethylnaphthalene. ¹H NMR (500 MHz, CDCl₃) δ 8.27 (d, J=7.3 Hz, 1H), 8.20 (d, J=8.5 Hz, 1H), 7.99-8.00 (m, 1H), 7.84-7.86 (m, 1H), 7.76 (d, J=8.2 Hz, 1H), 7.56 (t, J=7.9 Hz, 1H), 7.45-7.46 (m, 1H), 6.76-6.78 (m, 4H), 6.72 (d, J=8.5 Hz, 1H), 6.39 (s, 1H), 5.54 (s, 1H), 4.21 (br s, 1H), 3.80 (s, 3H), 2.19 (d, J=1.2 Hz, 3H), 1.39 (s, 6H).

### Example 97

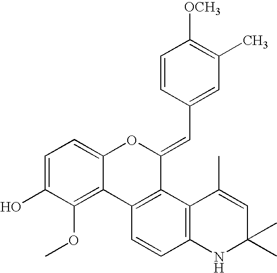

(Z)-5-(3′-methyl-4′-methoxybenzylidene)-1,2-dihydro-9-hydroxy-10-methoxy-2,2-cyclohexyl-4-methyl-5H-chromeno[3,4-f]quinoline (Compound 112, structure 1 of Scheme I, where R¹=4-methoxy-3-methylphenyl)

This compound was prepared according to General Method 1 (Example 1) from 4-methoxy-3-methylbenzyl bromide. ¹H NMR (500 MHz, CDCl₃) δ 8.13 (d, J=8.6 Hz, 1H), 7.63 (m, 1H), 7.54 (m, 1H,), 6.89 (d, J=7.9 Hz, 1H), 6.83 (d, J=8.6 Hz, 1H), 6.66 (d, J=8.6 Hz, 1H), 6.14 (s, 1H), 5.56 (s, 1H), 5.50 (m, 2H), 4.16 (s, 1H), 3.85 (s, 3H), 3.81 (s, 3H), 2.25 (s, 3H), 2.09 (s, 3H), 1.35 (br s, 6H).

### Example 98

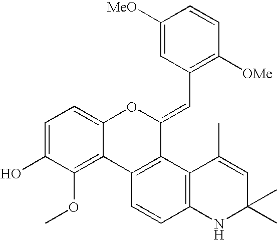

(Z)-5-(2′,5′-dimethoxybenzylidene)-1,2-dihydro-9-hydroxy-10-methoxy-2,2-4-methyl-5H-chromeno[3,4-f]quinoline (Compound 113, structure 1 of Scheme I, where R¹=2,5-dimethoxyphenyl)

This compound was prepared according to General Method 1 (Example 1) from 2,5-dimethoxybenzyl bromide. ¹H NMR (400 MHz, CD₃Cl) δ 8.13 (d, J=8.6 Hz, 1H), 7.88 (d, J=7.9 Hz, 1H), 7.30 (s, 1H), 6.88 (m, 3H), 6.67 (d, J=8.6 Hz, 1H), 6.07 (s, 1H,), 5.56 (s, 1H), 5.51 (m, 2H), 4.24 (s, 1H), 3.87 (s, 3H), 3.77 (s, 3H), 3.75 (s, 3H), 2.12 (s, 3H), 1.35 (br s, 6H).

### Example 99

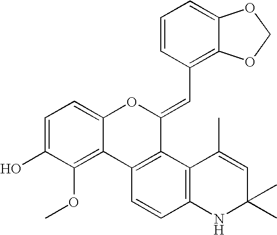

(Z)-5-(2′,3′-methylenedioxybenzylidene)-1,2-dihydro-9-hydroxy-10-methoxy-2,2,4-trimethyl-5H-chromeno[3,4-f]quinoline (Compound 114, structure 1 of Scheme I, where R¹=2,3-methylenedioxyphenyl)

This compound was prepared according to General Method 1 (Example 1) from 2,3-methylenedioxybenzyl bromide. ¹H NMR (500 MHz, CDCl₃) δ 8.17 (s, 1H), 7.72 (m, 2H), 6.84 (t, J=7.9 Hz, 1H), 6.70 (d, J=8.6 Hz, 2H), 5.92 (s, 1H), 5.88 (s, 1H), 5.55 (m, 2H), 5.29 (s, 2H), 3.79 (s, 3H), 2.11 (s, 3H), 1.34 (br s, 6H).

### Example 100

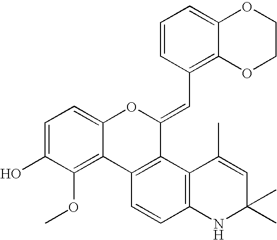

(Z)-5-(2′,3′-ethylenedioxybenzylidene)-1,2-dihydro-9-hydroxy-10-methoxy-2,2,4-trimethyl-5H-chromeno[3,4-f]quinoline (Compound 115, structure 1 of Scheme I, where R¹=2,3-ethylenedioxyphenyl)

This compound was prepared according to General Method 1 (Example 1) from 2,3-ethylenedioxybenzyl bromide. ¹H NMR (500 MHz, CD₃OD) δ 8.27 (m, 2H), 8.04 (s, 1H), 6.88 (m, 1H), 6.72 (m, 2H), 5.88 (s, 1H), 5.45 (m, 2H), 3.73 (s, 3H), 3.68 (m, 4H), 2.02 (s, 3H), 1.26 (br s, 6H).

### Example 101

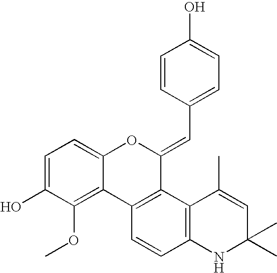

(Z)-5-(4′-hydroxybenzylidene)-1,2-dihydro-9-hydroxy-10-methoxy-2,2,4-trimethyl-5H-chromeno[3,4-f]quinoline (Compound 116, structure 1 of Scheme I, where R¹=4-hydroxyphenyl)

This compound was prepared according to General Method 1 (Example 1) from 4-trimethylsiloxybenzyl bromide. ¹H NMR (500 MHz, CD₃OD) δ 8.22 (d, J=8.6 Hz, 1H), 7.56-7.54 (m, 2-overlapping signals, 2H), 6.78-6.74 (m, 3H), 6.70-6.67 (m, 2H), 5.47 (s, 1H), 5.44 (s, 1H), 3.72 (s, 3H), 2.02 (s, 3H), 1.28 (br s, 6H).

### Example 102

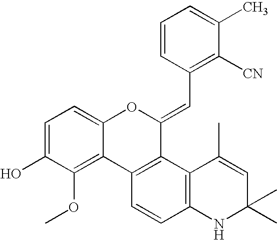

(Z)-5-(2′-cyano-3′-methylbenzylidene)-1,2-dihydro-9-hydroxy-10-methoxy-2,2,4-trimethyl-5H-chromeno[3,4-f]quinoline (Compound 117, structure 1 of Scheme I, where R¹=2-cyano-3-methylphenyl)

This compound was prepared according to General Method 2 (Example 60) from 2,6-dimethyl-benzonitrile. ¹H NMR (400 MHz, CDCl₃) δ 8.27 (d, J=8.1 Hz, 1H), 8.20 (d, J=8.6 Hz, 1H), 7.47 (t, J=7.9 Hz, 1H), 7.11 (d, J=7.9 Hz, 1H), 6.88 (d, J=8.6 Hz, 1H), 6.72 (d, J=8.6 Hz, 1H), 6.14 (s, 1H), 5.59 (m, 2H), 4.24 (s, 1H), 3.81 (s, 3H), 2.53 (s, 3H), 2.12 (s, 3H), 1.37 (br s, 6H).

### Example 103

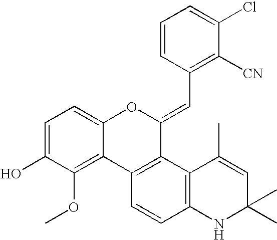

(Z)-5-(3′-chloro-2′-cyanobenzylidene)-1,2-dihydro-9-hydroxy-10-methoxy-2,2,4-trimethyl-5H-chromeno[3,4-f]quinoline (Compound 118, structure 1 of Scheme I, where R¹=3-chloro-2-cyanophenyl)

This compound was prepared according to General Method 2 (Example 60) from 2-chloro-6-methylbenzonitrile. ¹H NMR (400 MHz, CDCl₃) δ 8.37 (d, J=8.6 Hz, 1H), 8.23 (d, J=7.9 Hz, 1H), 7.49 (t, J=7.9 Hz, 1H), 7.29 (s, 1H), 6.88 (d, J=8.6 Hz, 1H), 6.76 (d, J=8.6 Hz, 1H), 6.12 (s, 1H), 5.59 (m, 2H), 4.26 (s, 1H), 3.81 (s, 3H), 2.11 (s, 3H), 1.37 (br s, 6H).

### Example 104

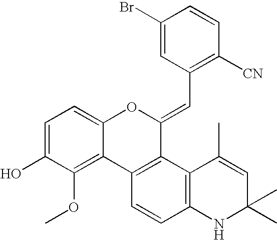

(Z)-5-(5′-bromo-2′-cyano-benzylidene)-1,2-dihydro-9-hydroxy-10-methoxy-2,2,4-trimethyl-5H-chromeno[3,4-f]quinoline (Compound 119, structure 1 of Scheme I, where R¹=5-bromo-2-cyanophenyl)

This compound was prepared according to General Method 2 (Example 60) from 4-bromo-2-methylbenzonitrile. ¹H NMR (500 MHz, CDCl₃) δ 8.69 (m, 1H), 8.24 (d, J=8.2 Hz, 1H), 7.45 (m, 1H), 7.36 (m, 1H), 6.93 (m, 2H), 6.75 (m, 1H), 6.07 (s, 1H), 5.60 (m, 2H), 4.22 (s, 1H), 3.81 (s, 3H), 2.10 (s, 3H), 1.36 (br s, 6H).

### Example 105

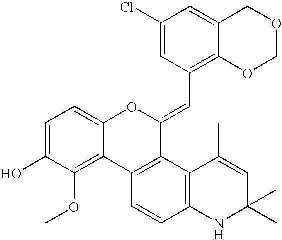

(Z)-5-(8′-(6′-chloro-benzo-1′,3′-dioxan-methylidiene))-1,2-dihydro-9-hydroxy-10-methoxy-2,2,4-trimethyl-5H-chromeno[3,4-f]quinoline (Compound 120, structure 1 of Scheme I, where R¹=8-(6-chloro-benzo-1,3-dioxan))

This compound was prepared according to General Method 1 (Example 1) from 6-chloro-8-chloromethylbenzo-1,3-dioxane. ¹H NMR (400 MHz, CD₃OD) δ 8.27 (d, J=8.8 Hz, 1H), 8.04 (d, J=2.5 Hz), 1H), 6.85 (d, J=2.4 Hz, 1H), 6.74-6.71 (m, 3H), 5.89 (s, 1H), 5.45 (s, 1H), 5.19 (s, 2H), 4.81 (s, 2H), 3.73 (s, 3H), 2.03 (s, 3H), 1.27 (br s, 6H).

### Example 106

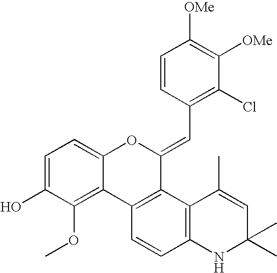

(Z)-5-(2′-chloro-3′,4′-dimethoxybenzylidene)-1,2-dihydro-9-hydroxy-10-methoxy-2,2,4-trimethyl-5H-chromeno[3,4-f]quinoline (Compound 121, structure 1 of Scheme I, where R¹=2-chloro-3,4-dimethoxyphenyl)

This compound was prepared according to General Method 1 (Example 1) from 2-chloro-3,4-dimethoxybenzyl bromide. ¹H NMR (400 MHz, CDCl₃) δ 8.27 (d, J=8.6 Hz, 1H), 8.21 (d, J=7.9 Hz, 1H), 7.49 (t, J=7.9 Hz, 1H), 7.11 (d, J=7.9 Hz, 1H), 6.88 (d, J=8.6 Hz, 1H), 6.72 (d, J=8.6 Hz, 1H), 6.14 (s, 1H), 5.88 (s, 1H), 5.59 (s, 1H), 3.96 (s, 3H), 3.81 (s, 3H), 3.55 (s, 3H), 2.12 (s, 3H), 1.37 (br s, 6H).

### Example 107

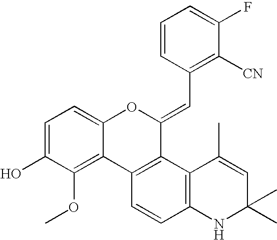

(Z)-5-(2′-cyano-3′-fluorobenzylidene)-1,2-dihydro-9-hydroxy-10-methoxy-2,2,4-trimethyl-5H-chromeno[3,4-f]quinoline (Compound 122, structure 1 of Scheme I, where R¹=2-cyano-3-fluorophenyl)

This compound was prepared according to General Method 2 (Example 60) from 2-fluoro-6-methylbenzonitrile. ¹H NMR (400 MHz, CDCl₃) δ 8.26 (m, 2H), 7.55 (m, 1H), 6.98 (m, 1H), 6.88 (m, 2H), 6.72 (m, 2H), 6.08 (s, 1H), 5.61 (s, 1H), 3.81 (s, 3H), 2.11 (s, 3H), 1.37 (br s, 6H).

### Example 108

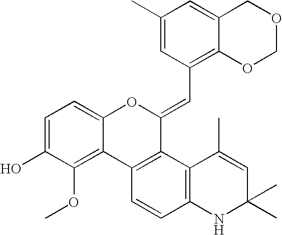

(Z)-5-(8′(6′-methyl-benzo-1′,3′-dioxan-methylidiene))-1,2-dihydro-9-hydroxy-10-methoxy-2,2,4-trimethyl-5H-chromeno[3,4-f]quinoline (Compound 123, structure 1 of Scheme I, where R¹=8-(6-methyl-benzo-1,3-dioxan))

This compound was prepared according to General Method 1 (Example 1) from 8-chloromethyl-6-methyl-benzo-1,3-dioxane. ¹H NMR (400 MHz, CD₃OD) δ 8.23 (d, J=8.7 Hz, 1H), 7.85 (s, 1H), 6.72-6.64 (m, 4H), 5.89 (s, 1H), 5.45 (s, 1H), 5.15 (s, 2H), 4.79 (s, 2H), 3.73 (s, 3H), 2.28 (s, 3H), 2.05 (s, 3H), 1.26 (br s, 6H).

### Example 109

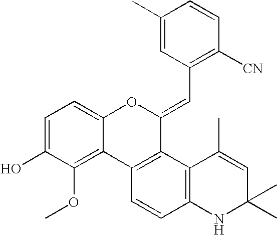

(Z)-5-(2′-cyano-5′-methylbenzylidene)-1,2-dihydro-9-hydroxy-10-methoxy-2,2,4-trimethyl-5H-chromeno[3,4-f]quinoline (Compound 124, structure 1 of Scheme I, where R¹=2-cyano-5-methylphenyl)

This compound was prepared according to General Method 2 (Example 60) from 2,4-dimethylbenzonitrile. ¹H NMR (500 MHz, CDCl₃) δ 8.22 (m, 1H), 7.67 (m, 2H), 7.54 (m, 1H), 6.88 (m, 1H), 6.66 (m, 2H), 5.60 (m, 2H), 5.53 (s, 1H), 4.22 (s, 1H), 3.81 (s, 3H), 2.56 (s, 3H), 2.07 (s, 3H), 1.36 (br s, 6H).

### Example 110

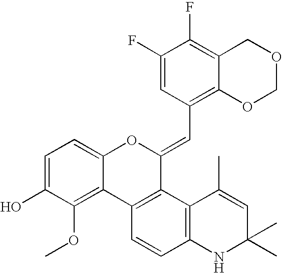

(Z)-5-(8′-(5′,6′-difluoro-benzo-1′,3′-dioxan-methylidiene))-1,2-dihydro-9-hydroxy-10-methoxy-2,2,4-trimethyl-5H-chromeno[3,4-f]quinoline (Compound 125, structure 1 of Scheme I, where R¹=8-(5,6-difluoro-benzo-1,3-dioxan))

This compound was prepared according to General Method 1 (Example 1) from 8-chloromethyl-5,6-difluoro-benzo-1,3-dioxane. ¹H NMR (400 MHz, CD₃Cl) δ 8.23 (d, J=8.7 Hz, 1H), 7.95 (m, 1H), 6.72-6.64 (m, 4H), 5.89 (s, 1H), 5.55 (s, 1H), 5.15 (s, 2H), 4.86 (s, 2H), 3.73 (s, 3H), 2.03 (s, 3H), 1.26 (br s, 6H).

### Example 111

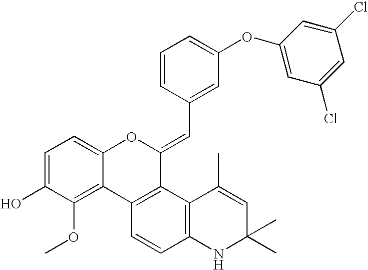

(Z)-5-(3′-(3″,5″-dichlorophenoxy)benzylidene)-1,2-dihydro-9-hydroxy-10-methoxy-2,2,4-trimethyl-5H-chromeno[3,4-f]quinoline (Compound 126, structure 1 of Scheme I, where R¹=3-(3′,5′-dichlorophenoxy)phenyl)

This compound was prepared according to General Method 1 (Example 1) from 3-(3′,5′-dichlorophenoxy)benzyl bromide. ¹H NMR (400 MHz, CD₃OD) δ 8.25 (d, J=8.6 Hz, 1H), 7.60 (s, 1H), 7.34 (t, J=7.9 Hz, 1H), 7.25 (d, J=7.7 Hz, 1H), 7.21 (t, J=1.6 Hz, 1H), 6.98 (s, 2H), 6.89 (dd, Z=7.8, 1.7 Hz, 1H), 6.72 (d, J=8.7 Hz, 1H), 6.63 (d, J=8.9 Hz, 1H), 6.37 (d, J=9.9 Hz, 1H), 5.55 (s, 1H), 5.47 (s, 1H), 3.72 (s, 3H), 2.02 (s, 3H), 1.26 (br s, 6H).

### Example 112

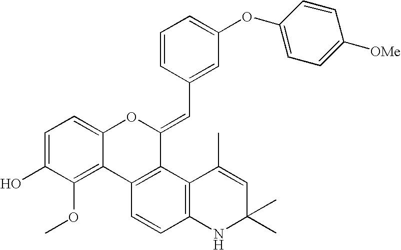

(Z)-5-(3′-(4″-methoxyphenoxy)benzylidene)-1,2-dihydro-9-hydroxy-10-methoxy-2,2,4-trimethyl-5H-chromeno[3,4-f]quinoline (Compound 127, structure 1 of Scheme I, where R¹=3-(4′-methoxyphenoxy)phenyl)

This compound was prepared according to General Method 1 (Example 1) from 3-(4′-methoxyphenoxy)benzyl bromide. ¹H NMR (400 MHz, CDCl₃) δ 8.13 (d, J=8.6 Hz, 1H), 7.57 (s, 1H), 7.25-7.23 (m, 1H), 7.07-7.05 (m, 2-overlapping signals, 2H), 6.95-6.92 (m, 2-overlapping signals, 2H), 6.85 (d, J=7.9 Hz, 1H), 6.73 (d, J=8.7 Hz, 1H), 6.65 (d, J=8.6 Hz, 1H), 6.43 (d, J=8.7 Hz, 1H), 5.56 (s, 1H), 5.53 (s, 1H), 5.51 (s, 1H), 4.17 (s, 1H), 3.85 (s, 3H), 3.76 (s, 3H), 2.08 (s, 3H), 1.34 (br s, 6H).

### Example 113

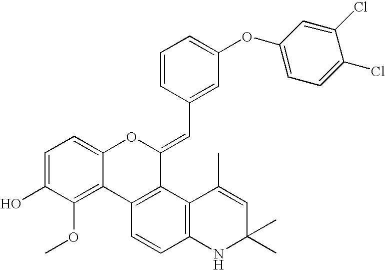

(Z)-5-(3′-(3″,4″-dichlorophenoxy)benzylidene)-1,2-dihydro-9-hydroxy-10-methoxy-2,2,4-trimethyl-5H-chromeno[3,4-f]quinoline (Compound 128, structure 1 of Scheme I, where R¹=3-(3′,4′-dichlorophenoxy)phenyl)

This compound was prepared according to General Method 1 (Example 1) from 3-(3′,4′-dichlorophenoxy)benzyl bromide. ¹H NMR (400 MHz, CDCl₃) δ 8.15 (d, J=8.6 Hz, 1H), 7.57 (s, 1H), 7.43-7.39 (m, 2H), 7.33 (t, J=7.9 Hz, 1H), 7.16 (d, J=2.7 Hz, 1H), 6.93 (dd, J=8.8, 2.8 Hz, 1H), 6.89 (d, J=7.8 Hz, 1H), 6.80 (d, J=8.8 Hz, 1H), 6.67 (d, J=8.6 Hz, 1H), 6.56 (d, J=8.7 Hz, 1H) 5.58 (s, 1H), 5.56 (s, 1H), 5.51 (s, 1H), 4.19 (s, 1H), 3.77 (s, 3H), 2.08 (s, 3H), 1.35 (br s, 6H).

### Example 114

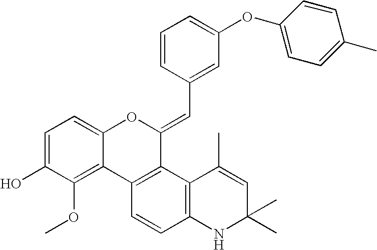

(Z)-5-(3′-(4″-methylphenoxy)benzylidene)-1,2-dihydro-9-hydroxy-10-methoxy-2,2,4-trimethyl-5H-chromeno[3,4-f]quinoline (Compound 129, structure 1 of Scheme I, where R¹=3-(4′-methylphenoxy)phenyl)

This compound was prepared according to General Method 1 (Example 1) from 3-(4′-methylphenoxy)benzyl bromide. ¹H NMR (400 MHz, CDCl₃) δ 8.13 (d, J=8.6 Hz, 1H), 7.59 (s, 1H), 7.31-7.27 (m, 2H), 7.20-7.18 (m, 2-overlapping signals, 2H), 7.01-6.99 (m, 2-overlapping signals, 2H), 6.89-6.87 (m, 1H), 6.72 (d, J=8.9 Hz, 1H), 6.65 (d, J=8.6 Hz, 1H), 6.43 (d, J=8.9 Hz, 1H), 5.56 (s, 1H), 5.55 (s, 1H), 5.51 (s, 1H), 4.17 (s, 1H), 3.75 (s, 3H), 2.39 (s, 3H), 2.08 (s, 3H), 1.34 (br s, 6H).

### Example 115

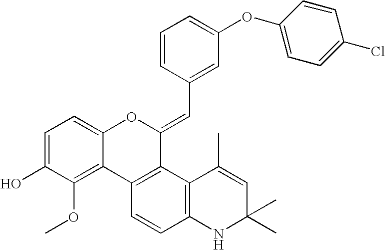

(Z)-5-(3′-(4″-chlorophenoxy)benzylidene)-1,2-dihydro-9-hydroxy-10-methoxy-2,2,4-trimethyl-5H-chromeno[3,4-f]quinoline (Compound 130, structure 1 of Scheme I, where R¹=3-(4′-chlorophenoxy)phenyl)

This compound was prepared according to General Method 1 (Example 1) from 3-(4′-chlorophenoxy)benzyl bromide. ¹H NMR (400 MHz, CDCl₃) δ 8.14 (d, J=8.6 Hz, 1H), 7.59 (s, 1H), 7.36-7.28 (m, 4H), 7.04-7.01 (m, 2-overlapping signals, 2H), 6.89 (d, J=8.7 Hz, 1H), 6.79 (d, J=8.9 Hz, 1H), 6.66 (d, J=8.6 Hz, 1H), 6.47 (d, J=8.9 Hz, 1H), 5.57 (s, two overlapping signals, 2H), 5.51 (s, 1H), 4.15 (s, 1H), 3.77 (s, 3H), 2.08 (s, 3H), 1.34 (br s, 6H).

### Example 116

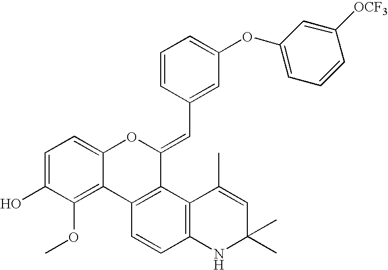

(Z)-5-(3′-(3″-trifluoromethoxyphenoxy)benzylidene)-1,2-dihydro-9-hydroxy-10-methoxy-2,2,4-trimethyl-5H-chromeno[3,4-f]quinoline (Compound 131, structure 1 of Scheme I, where R¹=3-(3′-trifluoromethoxyphenoxy)phenyl)

This compound was prepared according to General Method 1 (Example 1) from 3-(3′-trifluoromethoxyphenoxy)benzyl bromide. ¹H NMR (400 MHz, CDCl₃) δ 8.15 (d, J=8.6 Hz, 1H), 7.58 (t, J=1.7 Hz, 1H), 7.49-7.32 (m, 5H), 7.23 (d, J=8.2 Hz, 1H), 6.90 (d, J=7.9 Hz, 1H), 6.75 (d, J=8.9 Hz, 1H), 6.67 (d, J=8.6 Hz, 1H), 6.55 (d, J=8.7 Hz, 1H), 5.59 (s, 1H), 5.56 (s, 1H), 5.51 (s, 1H), 4.19 (s, 1H), 3.76 (s, 3H), 2.09 (s, 3H), 1.34 (br s, 6H).

### Example 117

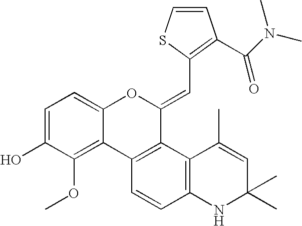

(Z)-5-(2′-(3′-(dimethylaminocarbonyl)thienylmethylidene))-1,2-dihydro-9-hydroxy-10-methoxy-2,2,4-trimethyl-5H-chromeno[3,4-f]quinoline (Compound 132, structure 1 of Scheme I, where R¹=2-(3-dimethylaminocarbonyl)thienyl)

This compound was prepared according to General Method 2 (Example 60) from N,N-dimethyl-2-methyl-3-thienylamide. ¹H NMR (500 MHz, CD₃OD) δ 8.34 (d, J=8.8 Hz, 1H), 7.40 (d, J=5.4 Hz, 1H), 6.98 (dd, J=5.4, 1.0 Hz, 1H), 6.95 (d, J=8.8 Hz, 1H), 6.77-6.74 (m, 2H), 5.87 (s, 1H), 5.51 (d, J=1.0 Hz, 1H), 3.76 (s, 3H), 3.05 (s, 3H), 2.89 (s, 3H), 2.00 (s, 3H), 1.29 (br s, 6H).

### Example 118

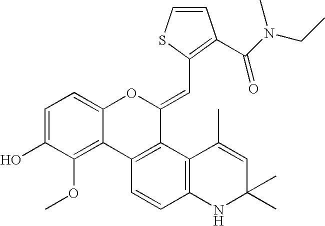

(Z)-5-(2′-(3′-(ethylmethylaminocarbonyl)thienylmethylidene))-1,2-dihydro-9-hydroxy-10-methoxy-2,2,4-trimethyl-5H-chromeno[3,4-f]quinoline (Compound 134, structure 1 of Scheme I, where R¹=2-(3-ethylmethylaminocarbonyl)thienyl)

This compound was prepared according to General Method 2 (Example 60) from N-ethyl-N-methyl-2-methyl-3-thienylamide. ¹H NMR (500 MHz, CD₃OD) δ 8.33 (d, J=8.3 Hz, 1H), 7.40 (d, J=4.9 Hz, 1H), 6.99-6.94 (m, 2H), 6.76 (d, J=3.4 Hz, 1H), 6.75 (d, J=3.4 Hz, 1H), 5.88 (d, J=8.3 Hz, 1H) 5.51-5.48 (m, 2H), 3.76 (s, 3H), 3.53-3.51 (m, 1H), 3.24 (q, J=6.6 Hz, 1H), 3.02 (s, 1.4H), 2.87 (s, 1.6H), 2.01 (m, 3H, rotamers), 1.28 (br s 6H), 1.22 (t, J=7.1 Hz, 1.4H), 1.03 (t, J=7.1 Hz, 1.6H).

### Example 119

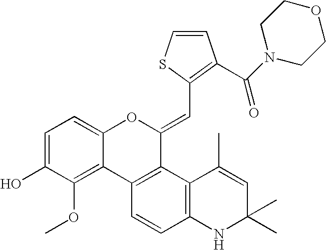

(Z)-5-(2′-(3′-(morpholinocarbonyl)thienylmethylidene))-1,2-dihydro-9-hydroxy-10-methoxy-2,2,4-trimethyl-5H-chromeno[3,4-f]quinoline (Compound 135, structure 1 of Scheme I, where R¹=2-(3-morpholinocarbonyl)thienyl)

This compound was prepared according to General Method 2 (Example 60) from 2-methyl-3-(morpholinecarbonyl)thiophene. ¹H NMR (500 MHz, CD₃OD) δ 8.34 (d, J=8.8 Hz, 1H), 7.41 (d, J=5.4 Hz, 1H), 7.00 (dd, J=5.4, 1H), 6.95 (d, J=8.8 Hz, 1H), 6.77 (d, J=8.3 Hz, 1H), 6.75 (d, J=8.8 Hz, 1H), 5.95 (s, 1H), 5.51 (d, J=1.5 Hz, 1H), 3.76 (s, 3H), 3.70-3.68 (m, 3H), 3.52-3.50 (m, 3H), 3.34-3.30 (m, 2H, partially obscured by solvent), 2.02 (d, J=1.0 Hz, 3H), 1.30 (br s, 6H).

### Example 120

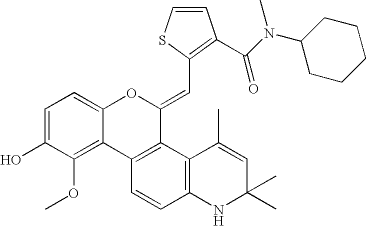

(Z)-5-(2′-(3′-(cyclohexylmethylaminocarbonyl)thienylmethylidene))-1,2-dihydro-9-hydroxy-10-methoxy-2,2,4-trimethyl-5H-chromeno[3,4-f]quinoline (Compound 136, structure 1 of Scheme I, where R¹=2-(3-(cyclohexylmethylaminocarbonyl)thienyl))

This compound was prepared according to General Method 2 (Example 60) from N-cyclohexyl-N-methyl-2-methyl-3-thienylamide. ¹H NMR (500 MHz, CD₃OD) δ 8.33 (d, J=8.8 Hz, 1H), 7.42 (app t, J=6.1 Hz, 1H), 6.99-6.94 (m, 2H), 6.76-6.75 (m, 2H), 5.85 (s, 1H), 5.45 (s, 1H), 4.39-4.37 (m, 1H), 3.75 (s, 3H), 3.41-3.38 (m, 1.5H), 2.95 (s, 1.5H), 2.75 (s, 1.5H), 2.01-1.99 (m, 3H, rotamers), 1.87-1.01 (m, 16H).

### Example 121

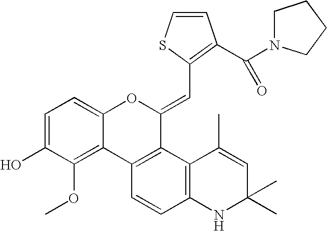

(Z)-5-(2′-(3′-pyrrolidinocarbonyl)thienylmethylidene))-1,2-dihydro-9-hydroxy-10-methoxy-2,2,4-trimethyl-5H-chromeno[3,4-f]quinoline (Compound 137, structure 1 of Scheme I, where R¹=2-(3-pyrrolidinocarbonyl)thienyl)

This compound was prepared according to General Method 2 (Example 60) from 2-methyl-3-(pyrrolidinecarbonyl)thiophene. ¹H NMR (400 MHz, CD₃OD) δ 8.34 (d, J=8.8 Hz, 1H), 7.38 (d, J=5.4 Hz, 1H), 7.01 (d, J=5.2 Hz, 1H), 6.95 (d, J=8.7 Hz, 1H), 6.77-6.74 (m, 2-overlapping signals, 2H), 5.95 (s, 1H), 5.48 (s, 1H), 3.76 (s, 3H), 3.52 (t, J=6.9 Hz, 2H), 3.24 (t, J=6.7 Hz, 2H), 2.00 (s, 3H), 1.91 (quintet, J=6.9 Hz, 2H), 1.85 (quintet, J=6.6 Hz, 2H), 1.27 (br s, 6H).

### Example 122

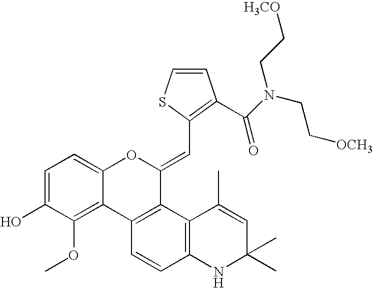

(Z)-5-(2′-(3′-(di(methoxyethyl)aminocarbonyl)thienylmethylidene))-1,2-dihydro-9-hydroxy-10-methoxy-2,2,4-trimethyl-5H-chromeno[3,4-f]quinoline (Compound 138, structure 1 of Scheme I, where R¹=2-(3-di(methoxyethyl)aminocarbonyl)thienyl)

This compound was prepared according to General Method 2 (Example 60) from N,N-dimethoxyethyl-2-methyl-3-thienylamide. ¹H NMR (500 MHz, CD₃OD) δ 8.38 (d, J=8.4 Hz, 1H), 7.40 (d, J=5.4 Hz, 1H), 7.01-6.98 (m, 2H), 6.79-6.70 (m, 2H), 5.87 (s, 1H), 5.53 (s, 1H), 3.80-3.53 (m, 7H), 3.41-3.39 (m, 5H), 3.30 (m, 2H, obscured by solvent), 3.13 (s, 3H), 2.00 (s, 3H), 1.29 (br s, 6H).

### Example 123

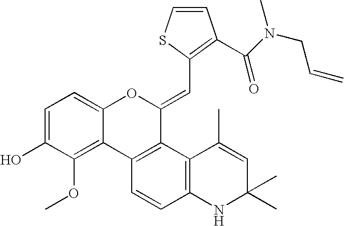

(Z)-5-(2′-(3′-(allylmethylaminocarbonyl)thienylmethylidene))-1,2-dihydro-9-hydroxy-10-methoxy-2,2,4-trimethyl-5H-chromeno[3,4-f]quinoline (Compound 139, structure 1 of Scheme I, where R¹=2-(3-allylmethylaminocarbonyl)thienyl)

This compound was prepared according to General Method 2 (Example 60) from N-allyl-N-methyl-2-methyl-3-thienylamide. ¹H NMR (500 MHz, CD₃OD) δ 8.34 (d, J=8.8 Hz, 1H), 7.40 (dd, J=12.4, 5.4 Hz, 1H), 7.01-6.95 (m, 2H), 6.78-6.75 (m, 2H), 5.93 (d, J=11.7 Hz, 1H), 5.87-5.83 (m, 1.2H, rotamer), 5.67-5.66 (m, 1.8H, rotamer), 5.50 (s, 1H), 5.27-5.08 (m, 2H), 4.10 (m, 0.6H), 3.81 (m, 0.4H), 3.76 (s, 3H), 3.00 (s, 1.2H), 2.81 (s, 1.8H), 2.02-2.00 (m, 3H), 1.29 (br s, 6H).

### Example 124

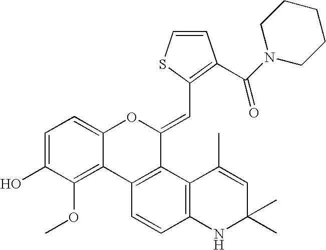

(Z)-5-(2′-(3′-(piperidinocarbonyl)thienylmethylidene))-1,2-dihydro-9-hydroxy-10-methoxy-2,2,4-trimethyl-5H-chromeno[3,4-f]quinoline (Compound 140, structure 1 of Scheme I, where R¹=2-(3-piperidinocarbonyl)thienyl)

This compound was prepared according to General Method 2 (Example 60) from 2-methyl-3-(piperidinecarbonyl)thiophene. ¹H NMR (500 MHz, CD₃OD) δ 8.34 (d, J=8.8 Hz, 1H), 7.40 (d, J=5.4 Hz, 1H), 6.97 (d, J=4.9 Hz, 1H), 6.96 (d, J=8.8 Hz, 1H), 6.77 (d, J=8.3 Hz, 1H), 6.75 (d, J=8.8 Hz, 1H), 5.93 (s, 1H), 5.50 (d, J=1.5 Hz, 1H), 3.77 (s, 3H), 3.68-3.65 (m, 2H), 3.30-3.27 (m, 2H, overlapping w/CD₃OH), 2.03 (d, J=1.0 Hz, 3H), 1.67-1.63 (m, 4H), 1.47-1.44 (m, 2H), 1.30 (br s, 6H).

### Example 125

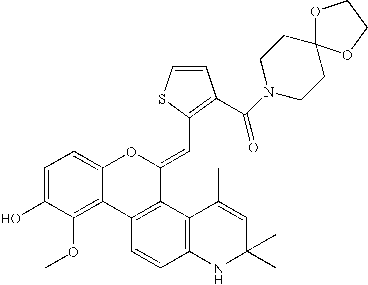

(Z)-5-(2′-(3′-piperidinecarbonyl-4″-(1,3-dioxan)thienylmethylidene))-1,2-dihydro-9-hydroxy-10-methoxy-2,2,4-trimethyl-5H-chromeno[3,4-f]quinoline (Compound 141, structure 1 of Scheme L where R¹=2-(3-piperidinecarbonyl-4′-(1,3-dioxan)carbonyl)thienyl)

This compound was prepared according to General Method 2 (Example 60) from 2-methyl-3-piperidinecarbonyl-4′-(1,3-dioxan)thienylamide. ¹H NMR (500 MHz, CD₃OD) δ 8.34 (d, J=8.8 Hz, 1H), 7.41 (d, J=5.4 Hz, 1H), 7.00 (d, J=5.4 Hz, 1H), 6.95 (d, J=8.8 Hz, 1H), 6.77 (d, J=8.8 Hz, 1H), 6.75 (d, J=8.8 Hz, 1H), 5.92 (s, 1H), 5.51 (d, J=1.5 Hz, 1H), 3.97-3.93 (m, 4H), 3.80-3.78 (m, 2H), 3.76 (s, 3H), 3.44-3.41 (m, 2H), 2.01 (d, J=1.5 Hz, 3H), 1.76-1.73 (m, 2H), 1.60-1.57 (m, 2H), 1.29 (br s, 6H).

### Example 126

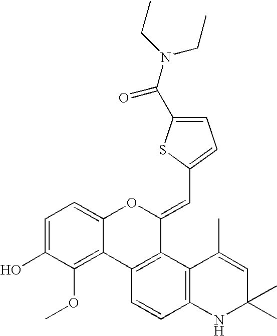

(Z)-5-(2′-(5′-(diethylaminocarbonyl)thienylmethylidene))-1,2-dihydro-9-hydroxy-10-methoxy-2,2,4-trimethyl-5H-chromeno[3,4-f]quinoline (Compound 142, structure 1 of Scheme I, where R¹=2-(5-diethylaminocarbonyl)thienyl)

This compound was prepared according to General Method 2 (Example 60) from N,N-diethyl-2-methyl-5-thienylamide. ¹H NMR (500 MHz, CD₃OD) δ 8.34 (d, J=8.3 Hz, 1H), 7.31 (d, J=3.9 Hz, 1H), 7.01 (d, J=3.9 Hz, 1H), 6.99 (d, J=8.8 Hz, 1H), 6.77 (d, J=8.8 Hz, 1H), 6.75 (d, J=8.8 Hz, 1H), 5.94 (s, 1H), 5.52 (d, J=1.5 Hz, 1H), 3.76 (s, 3H), 3.63-3.60 (m, 4H), 2.03 (d, J=1.5 Hz, 3H), 1.32-1.28 (m, 12H).

### Example 127

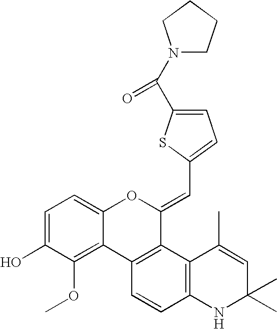

(Z)-5-(2′-(5′-(pyrrolidinocarbonyl)thienylmethylidene))-1,2-dihydro-9-hydroxy-10-methoxy-2,2,4-trimethyl-5H-chromeno[3,4-f]quinoline (Compound 143, structure 1 of Scheme I, where R¹=2-(5-pyrrolidinocarbonyl)thienyl)

This compound was prepared according to General Method 2 (Example 60) from 2-methyl-5-(pyrrolidinecarbonyl)thiophene. ¹H NMR (500 MHz, CD₃OD) δ 8.34 (d, J=8.3 Hz, 1H), 7.52 (d, J=3.9 Hz, 1H), 7.04 (d, J=3.9 Hz, 1H), 7.02 (d, J=8.8 Hz, 1H), 6.77 (d, J=8.8 Hz, 1H), 6.75 (d, J=8.8 Hz, 1H), 5.94 (s, 1H), 5.52 (s, 1H), 3.88-3.85 (m, 2H), 3.76 (s, 3H), 3.65-3.62 (m, 2H), 2.06-1.98 (m, 4H), 2.03 (d, J=1.5 Hz, 3H), 1.32-1.28 (m, 6H).

### Example 128

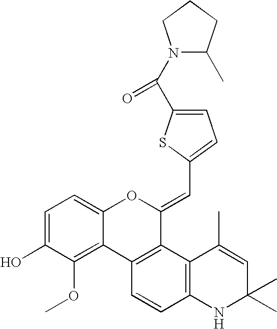

(Z)-5-(2′-(5′-(2″-methylpyrrolidinocarbonyl)thienylmethylidene))-1,2-dihydro-9-hydroxy-10-methoxy-2,2,4-trimethyl-5H-chromeno[3,4-f]quinoline (Compound 144, structure 1 of Scheme I, where R¹=2-(5-(2′-methylpyrrolidine)carbonylthienyl))

This compound was prepared according to General Method 2 (Example 60) from 2-methyl-5-(2-methylpyrrolidinecarbonyl)thiophene. ¹H NMR (500 MHz, CD₃OD) δ 8.34 (d, J=8.8 Hz, 1H), 7.49 (d, J=3.9 Hz, 1H), 7.03-7.01 (m, 2H), 6.77 (d, J=8.8 Hz, 1H), 6.75 (d, J=8.8 Hz, 1H), 5.93 (s, 1H), 5.52 (s, 1H), 4.35-4.31 (m, 1H), 3.90-3.85 (m, 2H), 3.77 (s, 3H), 2.16-2.08 (m, 2H), 2.03 (d, J=1.0 Hz, 3H), 2.0-1.93 (m, 1H), 1.77-1.66 (m, 1H), 1.31-1.27 (m, 9H).

### Example 129

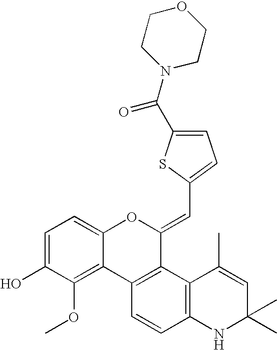

(Z)-5-(2′-(5′-(morpholinocarbonyl)thienylmethylidene))-1,2-dihydro-9-hydroxy-10-methoxy-2,2,4-trimethyl-5H-chromeno[3,4-f]quinoline (Compound 145, structure 1 of Scheme L where R¹=2-(5-morpholinocarbonyl)thienyl)

This compound was prepared according to General Method 2 (Example 60) from 2-methyl-5-(morpholinecarbonyl)thiophene. ¹H NMR (500 MHz, CD₃OD) δ 8.31 (d, J=8.8 Hz, 1H), 7.27 (d, J=3.9 Hz, 1H), 6.98 (d, J=3.9 Hz, 1H), 6.95 (d, J=8.8 Hz, 1H), 6.73 (d, J=8.8 Hz, 1H), 6.71 (d, J=8.8 Hz, 1H), 5.91 (s, 1H), 5.48 (d, J=1.5 Hz, 1H), 3.78-3.76 (m, 4H), 3.73 (s, 3H), 3.71-3.69 (m, 4H), 1.99 (d, J=1.0 Hz, 3H), 1.27 (m, 6H).

### Example 130

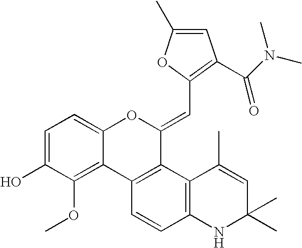

(Z)-5-(2′-(3′-dimethylaminocarbonyl-5H-methylfuranylmethylidene))-1,2-dihydro-9-hydroxy-10-methoxy-2,2,4-trimethyl-5H-chromeno[3,4-f]quinoline (Compound 146, structure 1 of Scheme I, where R¹=2-(5-methyl-3-dimethylaminocarbonyl)furanyl)

This compound was prepared according to General Method 2 (Example 60) from N,N-dimethyl-2,5-dimethyl-3-furanamide. ¹H NMR (500 MHz, CD₃OD) δ 8.33 (d, J=8.9 Hz, 1H), 6.78 (d, J=8.9 Hz, 1H), 6.75 (d, J=8.5 Hz, 1H), 6.70 (d, J=8.9 Hz, 1H), 6.16 (d, J=0.6 Hz, 1H), 5.58 (s, 1H), 5.49 (d, J=1.5 Hz, 1H), 3.75 (s, 3H), 3.00 (br s, 6H), 2.34 (d, J=0.9 Hz, 3H), 2.07 (d, J=1.5 Hz, 3H), 1.28 (br s, 6H).

### Example 131

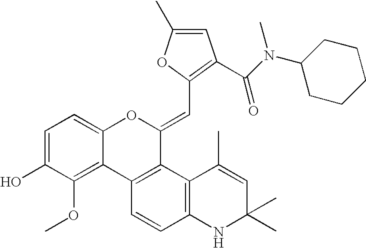

(Z)-5-(2′-(3′-cyclohexylmethylaminocarbonyl-5′-methylfuranylmethylidene))-1,2-dihydro-9-hydroxy-10-methoxy-2,2,4-trimethyl-5H-chromeno[3,4-f]quinoline (Compound 147, structure 1 of Scheme I, where R¹=2-(5-methyl-3-(cyclohexylmethylaminocarbonyl)furanyl))

This compound was prepared according to General Method 2 (Example 60) from N-cyclohexyl-N-methyl-2,5-dimethyl-3-furanamide. ¹H NMR (500 MHz, CD₃OD) δ 8.32 (d, J=8.9 Hz, 1H), 6.81 (d, J=8.9 Hz, 1H), 6.75 (d, J=8.5 Hz, 1H), 6.71 (d, J=8.9 Hz, 1H), 6.12 (m, 1H), 5.51 (s, 1H), 5.48 (d, J=1.2 Hz, 1H), 4.33-4.31 (m, 1H), 3.75 (s, 3H), 2.83 (s, 3H), 2.36 (d, J=0.9 Hz, 3H), 2.06 (d, J=1.5 Hz, 3H), 1.77-1.05 (m, 16H).

### Example 132

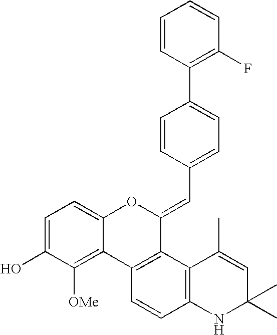

(Z)-5-(4′-(2″-Fluorophenyl)benzylidene)1,2-dihydro-9-hydroxy-10-methoxy-2,2,4-trimethyl-5H-chromeno[3,4-f]quinoline (Compound 148, structure 1 of Scheme I, where R¹=4-(2′-fluorophenyl)phenyl).

This compound was prepared according to General Method 1 (Example 1) from 4-(2′-fluorophenyl)benzyl bromide. ¹H NMR (500 MHz, CD₃OD) δ: 8.29 (d, J=8.85 Hz, 1H), 7.82-7.77 (m, 2-overlapping signals, 2H), 7.58-7.48 (m, 3H), 7.37-7.30 (m, 1H), 7.25-7.21 (m, 1H), 7.20-7.17 (m, 1H), 6.85 (d, J=8.85 Hz, 1H), 6.75 (d, J=8.85 Hz, 1H), 6.72 (d, J=8.85 Hz, 1H), 5.61 (s, 1H), 5.51 (s, 1H), 3.75 (s, 3H), 2.07 (s, 3H), 1.30 (bs, 6H).

### Example 133

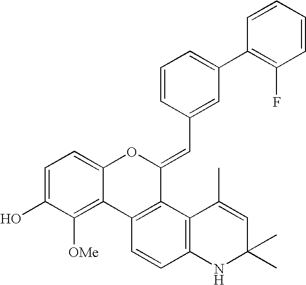

(Z)-5-(3′-(2″-Fluorophenyl)benzylidene)1,2-dihydro-9-hydroxy-10-methoxy-2,2,4-trimethyl-5H-chromeno[3,4-f]quinoline (Compound 149, structure 1 of Scheme I, where R¹=3-(2′-fluorophenyl)phenyl).

This compound was prepared according to General Method 1 (Example 1) from 3-(2′-fluorophenyl)benzyl bromide. ¹H NMR (CD₃OD) δ 1.32 (br s, 6H), 2.09 (s, 3H), 3.77 (s, 3H), 5.53 (s, 1H), 5.63 (s, 1H), 6.72 (d, J=8.5 Hz, 1H), 6.77 (d, J=8.5 Hz, 1H), 6.80 (d, J=8.9 Hz, 1H), 7.29 (ddd, J=1.2 Hz, 8.2 Hz, 11.0 Hz, 1H), 7.28 (dt, J=1.2 Hz, 7.3 Hz, 1H), 7.36-7.39 (m, 2H), 7.43 (t, J=7.6 Hz), 7.43 (t, J=7.6 Hz, 1H), 7.52 (dt, J=1.5 Hz, 7.6 Hz, 1H), 7.67 (d, J=7.9 Hz, 1H), 7.97 (d, J=1.5 Hz, 1H), 8.30 (d, J=8.9 Hz, 1H).

### Example 134

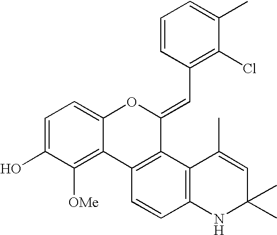

(Z)-5-(2′-Chloro-3′-methylbenzylidene)1,2-dihydro-9-hydroxy-10-methoxy-2,2,4-trimethyl-5H-chromeno[3,4-f]quinoline (Compound 150, structure 1 of Scheme I, where R¹=2-chloro-3-methylphenyl).

This compound was prepared according to General Method 1 (Example 1) from 2-chloro-3-methylbenzyl bromide. ¹H NMR (500 MHz, MeOD-d₄) δ 8.31 (d, J=8.9 Hz, 1H), 8.12 (dd, J=7.9, 1.2 Hz, 1H), 7.21 (app t, J=7.8 Hz, 1H), 7.12 (d, J=6.7 Hz, 1H), 6.79-6.71 (m, 3H), 6.15 (s, 1H), 5.50 (d, J=1.2 Hz, 1H), 3.77 (s, 3H), 2.36 (s, 3H), 2.09 (d, J=1.2 Hz, 3H), 1.31 (br s, 6H).

### Example 135

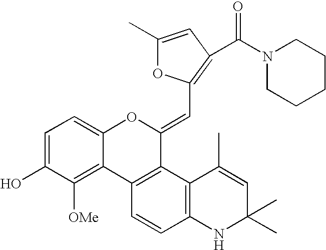

(Z)-5-(2′-(5′-Methyl-3′-(piperidinecarbonyl)furanylmethylidene))1,2-dihydro-9-hydroxy-10-methoxy-2,2,4-trimethyl-5H-chromeno[3,4-f]quinoline (Compound 151, Structure 1 of Scheme II, where R¹=2-(5-methyl-3-(piperidinecarbonyl)furanyl)).

General Method 3 TIPS protection of phenol. 2,6-Lutidine (4.5 equiv.) was added to a solution of phenol (1 equiv.) in dichloromethane (ca. 0.05 M). Triisopropylsilyl trifluoromethanesulfonate (2.3 equiv.) was added dropwise at room temperature and the reaction solution stirred for 72 h. The reaction was quenched with a saturated solution of ammonium chloride (10 mL/mmol), the layers separated and the aqueous layer extracted with dichloromethane (3×5 mL/mmol). The combined organic extracts were washed with a 1M hydrochloric acid solution (30 mL/mmol), a saturated solution of ammonium chloride (30 mL/mmol), dried (Na₂SO₄) and concentrated under reduced pressure. Purification by flash chromatography, eluting with ethyl acetate:hexanes afforded the TIPS protected phenol.

General Method 4: Lateral lithiation of an arylmethyl or heteroarylmethyl compound and addition to a lactone with a TBDMS-protected phenol, followed by dehydration. 2.5 M n-BuLi in hexanes (3.6 equiv.) was added dropwise to a solution of diisopropylamine (3.6 equiv.) in THF (sufficient to form ca. 0.6 M LDA soln) at 0° C. under a nitrogen atmosphere and the solution stirred for 0.2 h. A solution of arylmethyl or heteroarylmethyl compound (3.6 equiv.) in THF (0.5-1 M) was cooled to 0° C. and added dropwise over 0.5 h to the LDA solution via cannula. After complete addition the dark-red solution was stirred for an additional 0.2 h. This solution was added dropwise to a pre-cooled (0° C.) solution of lactone B1 (Scheme II) (1 equiv.) in THF (0.25 M) via cannula. On complete addition the reaction was stirred at room temperature for 15 h. The reaction was quenched with a saturated ammonium chloride solution, ethyl acetate was added, the layers separated and the aqueous layer extracted with ethyl acetate (3×). The combined organic extracts were washed with saturated ammonium chloride solution, dried (Na₂SO₄) and concentrated under reduced pressure. The oily foam was taken up in 1:1 hexanes:dichloromethane and the volume reduced slowly with cooling (620 mmHg, 1 h). The precipitate was filtered under reduced pressure and washed with hexanes (100 mL) to give the corresponding lactol. Alternatively, the lactol could be purified by silica gel chromatography (EtOAc:hexanes). The product is light sensitive. The lactol was dissolved in 10% v/v conc. HCl:methanol (3-5 mL/mmol) and stirred at room temperature for 15 h. Water was added, the suspension stirred for 0.1 h and the slurry filtered under reduced pressure. The precipitate was washed with water and ethyl acetate (100 mL). The off-white precipitate was taken up in 1:1 ethyl acetate:water (100 mL) and vigorously stirred for 1 h. The layers were separated, the organic layer washed with saturated sodium hydrogencarbonate solution (100 mL) and saturated ammonium chloride solution (100 mL), dried (Na₂SO₄) and concentrated under reduced pressure to yield I as a bright yellow powder. Alternatively, compound I could be isolated by flash chromatography (EtOAc:hexanes). Alternatively, compound I could be purified by HPLC (chromasil C18, methanol:water).

General Method 5: Lateral lithiation of an arylmethyl or heteroarylmethyl compound and addition to a protected lactone with a TIPS-protected phenol, followed by dehydration and silyl ether deprotection. The procedure was followed as described in General Method 4 except that the TIPS deprotection was carried out by treatment with TBAF. The TIPS-protected phenol (in THF, 0.01-0.1 M was treated by dropwise addition of TBAF (1M in THF, 3 equiv.) at 0° C. The reaction solution was stirred for 0.2 h at this temperature, a saturated solution of ammonium chloride (10 mL) added, ethyl acetate (10 mL) added and the layers separated. The aqueous layer was extracted with ethyl acetate (3×10 mL), the combined organic extracts washed with a saturated solution of ammonium chloride (30 mL), dried (Na₂SO₄) and concentrated under reduced pressure. Purification by flash chromatography, eluting with ethyl acetate:hexanes afforded the desired alcohol.

Preparation of 9-(tert-butyldimethylsilyl)oxy-10-methoxy-2,2,4-trimethyl-1,2-dihydro-5H-chromeno[3,4-f]quinoline-5-one (B1, Scheme II). Imidazole (6.66 g, 97.9 mmol) was added to a stirred solution of 9-hydroxy-10-methoxy-2,2,4-trimethyl-1,2-dihydro-5H-chromeno[3,4-f]quinoline-5-one (15.0 g, 44.5 mmol) in dry DMF (600 mL) under a nitrogen atmosphere. tert-Butyldimethylsilyl chloride (8.1 g, 53.4 mmol) was added in one portion and the solution stirred for 15 h at room temperature. The reaction solution was poured into water (1000 mL) and extracted with ethyl acetate (3×200 mL). The combined organic extracts were washed with a saturated ammonium chloride solution (800 mL), dried (MgSO₄) and concentrated under reduced pressure. Purification by flash chromatography, eluting with 4:1 hexanes:ethyl acetate, yielded 9-(tert-butyldimethylsilyl)oxy-10-methoxy-2,2,4-trimethyl-1,2-dihydro-5H-chromeno[3,4-f]quinoline-5-one as a bright yellow powder (15.9 g, 80%).

This compound was prepared according to General Method 4 (EXAMPLE 135) from (2,5-dimethylfuran-3-yl)-piperidine-1-yl-methanone and 9-(tert-butyldimethylsilyl)oxy-10-methoxy-2,2,4-trimethyl-1,2-dihydro-5H-chromeno[3,4-f]quinoline-5-one to afford Compound 151. ¹H NMR (500 MHz, Acetone-d₆) δ 8.31 (d, J=8.8 Hz, 1H), 7.76 (s, 1H), 6.88 (d, J=8.8 Hz, 1H), 6.80 (d, J=8.8 Hz, 1H), 6.77 (d, J=8.8 Hz, 1H), 6.14 (q, J=1.0 Hz, 1H), 5.89 (s, 1H), 5.69 (s, 1H), 5.51 (q, J=1.0 Hz, 1H), 3.76 (s, 3H), 3.49 (m, 4H), 2.36 (d, J=1.0 Hz, 3H), 2.10 (d, J=1.0 Hz, 3H), 1.61 (m, 2H), 1.48 (m, 4H), 1.31 (s, 6H).

### Example 136

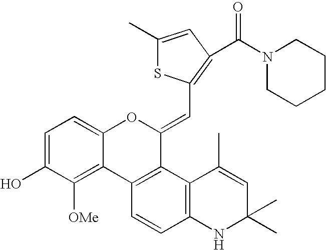

(Z)-5-(2′-(5′-Methyl-3′-(piperidinecarbonyl)thienylmethylidene))1,2-dihydro-9-hydroxy-10-methoxy-2,2,4-trimethyl-5H-chromeno[3,4-f]quinoline (Compound 152, Structure 1 of Scheme II, where R¹=5-methyl-3-(piperidinecarbonyl)thienyl)).

This compound was prepared according to General Method 4 (EXAMPLE 135) from 2,5-dimethyl-3-(piperidinecarbonyl)thiophene and 9-(tert-butyldimethylsilyl)oxy-10-methoxy-2,2,4-trimethyl-1,2-dihydro-5H-chromeno[3,4-f]quinoline-5-one to afford Compound 152. ¹H NMR (500 MHz, Acetone-d₆) [symbol] 8.31 (d, J=8.6 Hz, 1H), 7.83 (s, 1H), 6.99 (d, J=8.8 Hz, 1H), 6.81 (d, J=8.8 Hz, 1H), 6.79 (d, J=8.6 Hz, 1H), 6.66 (q, J=0.9 Hz, 1H), 5.97 (s, 1H), 5.91 (s, 1H), 5.51 (d, J=1.1 Hz, 1H), 3.77 (s, 3H), 3.60 (m, 2H), 3.30 (m, 2H), 2.50 (d, J=0.9 Hz, 3H), 2.04 (d, J=1.1 Hz, 3H), 1.63 (m, 2H), 1.56 (s, 2H), 1.44 (s, 2H), 1.32 (s, 6H).

### Example 137

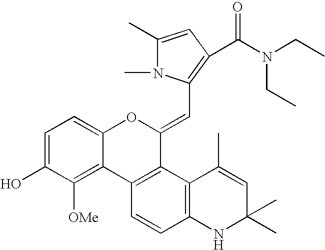

(Z)-5-(2′-(3′-Diethylcarbamoyl-1′,5′-dimethyl-1′H-pyrrolylmethylidene))1,2-dihydro-9-hydroxy-10-methoxy-2,2,4-trimethyl-5H-chromeno[3,4-f]quinoline (Compound 153, Structure 1 of Scheme II, where R¹=2-(3-diethylcarbamoyl-1,5-dimethyl-1H-pyrrole)).

This compound was prepared according to General Method 4 (EXAMPLE 135) from 3-diethylcarbamoyl-1,2,5-trimethyl-1H-pyrrole and 9-(tert-butyldimethylsilyl)oxy-10-methoxy-2,2,4-trimethyl-1,2-dihydro-5H-chromeno[3,4-f]quinoline-5-one to afford Compound 153. ¹H NMR (500 MHz, Acetone-d₆) δ 7.97 (s, 1H), 7.83 (d, J=8.3 Hz, 1H), 6.71 (d, J=8.7 Hz, 1H), 6.68 (d, J=8.7 Hz, 1H), 6.61 (d, J=8.3 Hz, 1H), 5.95 (q, J=0.9 Hz, 1H), 4.97 (s, 1H), 3.87 (s, 3H), 3.51 (s, 3H), 3.45 (m, 4H), 2.28 (s, 3H), 2.05 (m, 3H), 1.44 (s, 3H), 1.18 (s, 3H), 1.07 (m, 6H).

### Example 138

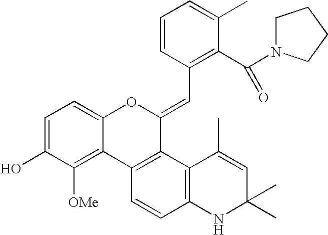

(Z)-5-(3′-Methyl-2′-(pyrrolidinecarbonyl)benzylidene)1,2-dihydro-9-hydroxy-10-methoxy-2,2,4-trimethyl-5H-chromeno[3,4-f]quinoline (Compound 154, Structure 1 of Scheme II, where R¹=3-methyl-2-(pyrrolidinecarbonyl)benzene.

This compound was prepared according to General Method 4 (EXAMPLE 135) from 1,3-dimethyl-2-(pyrrolidinecarbonyl)benzene and 9-(tert-butyldimethylsilyl)oxy-10-methoxy-2,2,4-trimethyl-1,2-dihydro-5H-chromeno[3,4-f]quinoline-5-one to afford Compound 154. ¹H NMR (500 MHz, CDCl₃) δ 8.26 (d, J=7.6 Hz, 1H), 8.14 (d, J=8.6 Hz, 1H), 7.30 (t, J=7.6 Hz, 1H), 7.06 (dq, J=7.6, 0.7 Hz, 1H), 6.82 (d, J=8.6 Hz, 1H), 6.78 (d, J=8.6 Hz, 1H), 6.66 (d, J=8.6 Hz, 1H), 5.70 (s, 1H), 5.51 (s, 1H), 5.48 (m, 1H), 4.19 (s, 1H), 3.75 (s, 3H), 3.62 (m, 1H), 3.53 (m, 1H), 3.11-2.96 (m, 2H), 2.25 (m, 3H), 2.10 (d, J=1.2 Hz, 3H), 1.95-1.76 (m, 4H), 1.34 (s, 3H), 1.31 (s, 3H).

### Example 139

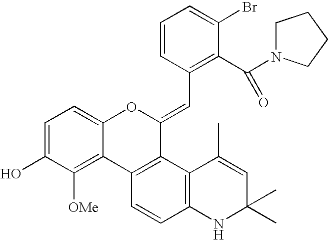

(Z)-5-(3′-Bromo-2′-(pyrrolidinecarbonyl)benzylidene)1,2-dihydro-9-hydroxy-10-methoxy-2,2,4-trimethyl-5H-chromeno[3,4-f]quinoline (Compound 155, Structure 1 of Scheme II, where R¹=3-bromo-2-(pyrrolidinecarbonyl)benzene).

This compound was prepared according to General Method 4 (EXAMPLE 135) from 3-bromo-2-(pyrrolidinecarbonyl)toluene and 9-(tert-butyldimethylsilyl)oxy-10-methoxy-2,2,4-trimethyl-1,2-dihydro-5H-chromeno[3,4-f]quinoline-5-one to afford Compound 155. ¹H NMR (500 MHz, CDCl₃) δ 8.41 (d, J=7.9 Hz, 1H), 8.17 (d, J=8.6 Hz, 1H), 7.39 (dd, J=7.9, 1.0 Hz, 1H), 7.26 (t, J=7.9 Hz, 1H), 6.83 (d, J=8.6 Hz, 1H), 6.79 (d, J=8.6 Hz, 1H), 6.68 (d, J=8.6 Hz, 1H), 5.78 (s, 1H), 5.52 (s, 1H), 5.49 (m, 1H), 4.25 (s, 1H), 3.76 (s, 3H), 3.65 (m, 1H), 3.52 (m, 1H), 3.23 (m, 1H), 3.05 (m, 1H), 2.08 (d, J=1.2 Hz, 3H), 1.98-1.80 (m, 4H), 1.35 (s, 3H), 1.30 (s, 3H).

### Example 140

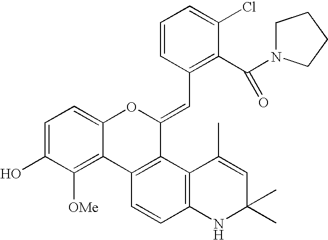

(Z)-5-(3′-Chloro-2′-(pyrrolidinecarbonyl)benzylidene)1,2-dihydro-9-hydroxy-10-methoxy-2,2,4-trimethyl-5H-chromeno[3,4-f]quinoline (Compound 156, Structure 1 of Scheme II, where R¹=3-chloro-2-(pyrrolidinecarbonyl)benzene).

This compound was prepared according to General Method 4 (EXAMPLE 135) from 3-chloro-2-(pyrrolidinecarbonyl)toluene and 9-(tert-butyldimethylsilyl)oxy-10-methoxy-2,2,4-trimethyl-1,2-dihydro-5H-chromeno[3,4-f]quinoline-5-one to afford Compound 156. ¹H NMR (500 MHz, CDCl₃) δ 8.37 (d, J=7.9 Hz, 1H), 8.18 (d, J=8.6 Hz, 1H), 7.33 (t, J=7.9 Hz, 1H), 7.22 (dd, J=7.9, 1.0 Hz, 1H), 6.82 (d, J=8.7 Hz, 1H), 6.78 (d, J=8.7 Hz, 1H), 6.68 (d, J=8.6 Hz, 1H), 6.05 (s, 1H), 5.51 (s, 1H), 5.48 (m, 1H), 4.32 (s, 1H), 3.75 (s, 3H), 3.65 (m, 1H), 3.52 (m, 1H), 3.22 (m, 1H), 3.04 (m, 1H), 2.08 (d, J=1.2 Hz, 3H), 1.93 (m, 4H), 1.33 (s, 3H), 1.29 (s, 3H).

### Example 141

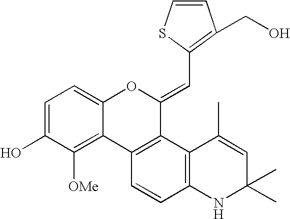

(Z)-5-(2′-(3′-Hydroxymethylthienylmethylidene))1,2-dihydro-9-hydroxy-10-methoxy-2,2,4-trimethyl-5H-chromeno[3,4-f]quinoline (Compound 157, Structure 9 of Scheme III, where X═S, R¹³═H).

General Method 6: Reduction of a tertiary amide to an alcohol. The product is light sensitive. 1M Lithium triethylborohydride in THF (5 equiv.) was added dropwise to a solution of the tertiary amide (1 equiv.) in THF at 0° C. The reaction solution was allowed to warm to room temperature and stirred for an additional 4 h. The reaction was quenched with the dropwise addition of a saturated solution of sodium hydrogencarbonate (20 mL/mmol), diluted with ethyl acetate (20 mL/mmol) and the layers separated. The aqueous layer was extracted with ethyl acetate (3×10 mL/mmol), the combined organics washed with a saturated solution of ammonium chloride (50 mL/mmol), dried (Na₂SO₄) and concentrated under reduced pressure. Purification by flash chromatography, eluting with ethyl acetate:hexanes afforded the desired alcohol.

(Z)-5-(2′-(3′-Hydroxymethylthienylmethylidene))1,2-dihydro-9-hydroxy-10-methoxy-2,2,4-trimethyl-5H-chromeno[3,4-f]quinoline (Compound 157, Structure 9 of Scheme II, where X═S, R¹³═H). This compound was prepared according to General Method 6 (EXAMPLE 141) from Compound 140 (EXAMPLE 124) to afford Compound 157 in 80% yield. ¹H NMR (500 MHz, Acetone-d₆) δ 8.30 (d, J=8.7 Hz, 1H), 7.78 (s, 1H), 7.32 (dd, J=5.3, 0.5 Hz, 1H), 7.06 (d, J=5.3 Hz, 1H), 6.99 (d, J=8.7 Hz, 1H), 6.80 (d, J=8.7 Hz, 1H), 6.79 (d, J=8.7 Hz, 1H), 6.20 (d, J=0.5 Hz, 1H), 5.88 (s, 1H), 5.53 (q, J=1.2 Hz, 1H), 4.62 (s, 2H), 3.76 (s, 3H), 2.06 (d, J=1.2 Hz, 3H), 1.33 (s, 6H).

### Example 142

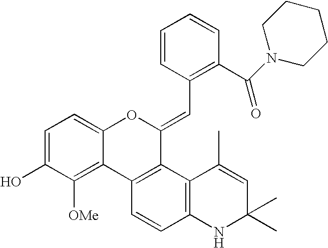

(Z)-5-(2′-(Piperidinecarbonyl)benzylidene)1,2-dihydro-9-hydroxy-10-methoxy-2,2,4-trimethyl-5H-chromeno[3,4-f]quinoline (Compound 158, Structure 1 of Scheme II, where R¹=2-(piperidinecarbonyl)phenyl).

This compound was prepared according to General Method 4 (EXAMPLE 135) from 2-(piperidinecarbonyl)toluene and 9-(tert-butyldimethylsilyl)oxy-10-methoxy-2,2,4-trimethyl-1,2-dihydro-5H-chromeno[3,4-f]quinoline-5-one to afford Compound 158. MS: 523.50 (MH+).

### Example 143

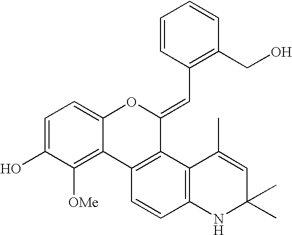

(Z)-5-(2′-Hydroxymethylbenzylidene)1,2-dihydro-9-hydroxy-10-methoxy-2,2,4-trimethyl-5H-chromeno[3,4-f]quinoline (Compound 159, Structure 37 of Scheme X, where R³═H, R⁴═H, R⁵═H).

This compound was prepared according to General Method 6 (EXAMPLE 141) from (Z)-5-(2′-(piperidinecarbonyl)benzylidine]-1,2-dihydro-9-hydroxy-10-methoxy-2,2,4-trimethyl-5H-chromeno[3,4-f]quinoline (Compound 158, EXAMPLE 142) to afford Compound 159. ¹H NMR (500 MHz, CDCl₃) δ 8.21 (dd, J=7.6, 1.0 Hz, 1H), 8.17 (d, J=8.6 Hz, 1H), 7.39 (m, 1H), 7.36 (dd, J=7.6, 1.2 Hz, 1H), 7.24 (td, J=7.6, 1.2 Hz, 1H), 6.81 (d, J=8.8 Hz, 1H), 6.79 (d, J=8.8 Hz, 1H), 6.70 (d, J=8.6 Hz, 1H), 5.94 (s, 1H), 5.52 (q, J=1.2 Hz, 1H), 4.69 (s, 2H), 4.21 (s, 1H), 3.80 (s, 3H), 2.14 (d, J=1.2 Hz, 3H), 1.36 (s, 6H).

### Example 144

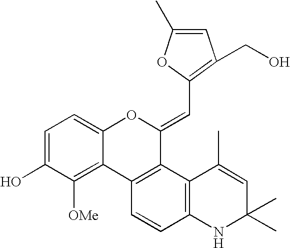

(Z)-5-(2′-(3′-(Hydroxymethyl)-5′-methylfuranylmethylidene))1,2-dihydro-9-hydroxy-10-methoxy-2,2,4-trimethyl-5H-chromeno[3,4-f]quinoline (Compound 160, Structure 9 of Scheme III, where X═O, R¹³=Me).

This compound was prepared according to General Method 6 (EXAMPLE 141) from Compound 151 (EXAMPLE 135) to afford Compound 160. ¹H NMR (500 MHz, CD₃OD) δ 8.27 (d, J=8.6 Hz, 1H), 6.76 (d, J=8.6 Hz, 1H), 6.73 (d, J=8.6 Hz, 1H), 6.69 (d, J=8.6 Hz, 1H), 6.12 (d, J=0.8 Hz, 1H), 5.52 (s, 1H), 5.50 (q, J=1.2 Hz, 1H), 4.47 (s, 2H), 3.74 (s, 3H), 2.33 (d, J=0.8 Hz, 3H), 2.07 (d, J=1.2 Hz, 3H), 1.29 (s, 6H).

### Example 145

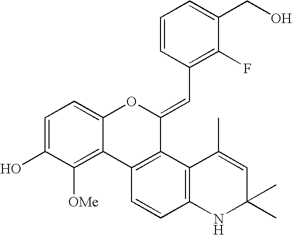

(Z)-5-(2′-Fluoro-3′-hydroxymethylbenzylidene)1,2-dihydro-9-hydroxy-10-methoxy-2,2,4-trimethyl-5H-chromeno[3,4-f]quinoline (Compound 161).

This compound was prepared according to General Method 4 (EXAMPLE 135) from 2-fluoro-3-(pyrrolidine-1-carbonyl)toluene and 9-(tert-butyldimethylsilyl)oxy-10-methoxy-2,2,4-trimethyl-1,2-dihydro-5H-chromeno[3,4-f]quinoline-5-one to afford (Z)-5-(2′-fluoro-3′-(pyrrolidinecarbonyl)benzylidine)1,2-dihydro-9-hydroxy-10-methoxy-2,2,4-trimethyl-5H-chromeno[3,4-f]quinoline. ¹H NMR (500 MHz, CDCl₃) δ 8.30 (ddd, J=7.3, 6.2, 3.6 Hz, 1H), 8.19 (d, J=8.6 Hz, 1H), 7.24-7.19 (m, 2H), 6.86 (d, J=8.6 Hz, 1H), 6.82 (d, J=8.6 Hz, 1H), 6.70 (d, J=8.6 Hz, 1H), 5.90 (s, 1H), 5.52 (q, J=1.2 Hz, 1H), 4.23 (s, 1H), 3.79 (s, 3H), 3.65 (t, J=7.0 Hz, 2H), 3.31 (t, J=6.7 Hz, 2H), 2.09 (d, J=1.2 Hz, 3H), 1.96 (m, 2H), 1.87 (m, 2H), 1.34 (s, 6H).

(Z)-5-(2′-Fluoro-3′-hydroxymethylbenzylidene)1,2-dihydro-9-hydroxy-10-methoxy-2,2,4-trimethyl-5H-chromeno[3,4-f]quinoline (Compound 161) was prepared according to General Method 6 (EXAMPLE 141) from (Z)-5-(2′-fluoro-3′-(pyrrolidinecarbonyl)benzylidine)1,2-dihydro-9-hydroxy-10-methoxy-2,2,4-trimethyl-5H-chromeno[3,4-f]quinoline to afford Compound 161. ¹H NMR (500 MHz, CDCl₃) δ 8.22 (td, J=7.6, 1.8 Hz, 1H), 8.18 (d, J=8.6 Hz, 1H), 7.26 (td, J=7.6, 1.8 Hz, 1H), 7.19 (t, J=7.6 Hz, 1H), 6.86 (d, J=8.6 Hz, 1H), 6.82 (d, J=8.6 Hz, 1H), 6.69 (d, J=8.6 Hz, 1H), 5.92 (s, 1H), 5.59 (s, 1H), 5.53 (q, J=1.2 Hz, 1H), 4.76 (d, J=6.0 Hz, 2H), 4.21 (s, 1H), 3.79 (s, 3H), 2.12 (d, J=1.2 Hz, 3H), 1.76 (t, J=6.2 Hz, 1H), 1.35 (s, 6H).

### Example 146

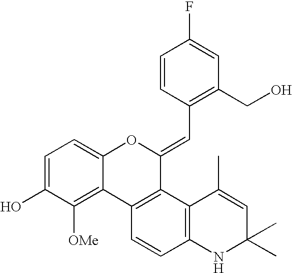

(Z)-5-(4′-Fluoro-2′-hydroxymethylbenzylidene)1,2-dihydro-9-hydroxy-10-methoxy-2,2,4-trimethyl-5H-chromeno[3,4-f]quinoline (Compound 162, Structure 37 of Scheme X, where R³═H, R⁴═F, R⁵═H).

This compound was prepared according to General Method 6 (EXAMPLE 141) from Compound 94 (EXAMPLE 79) to afford Compound 162). MS (Electrospray) 460.55 (MH+)

### Example 147

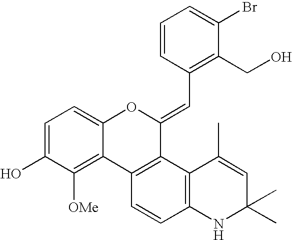

(Z)-5-(3′-Bromo-2′-hydroxymethylbenzylidene)1,2-dihydro-9-hydroxy-10-methoxy-2,2,4-trimethyl-5H-chromeno[3,4-f]quinoline (Compound 163, Structure 37 of Scheme X, where R³═Br, R⁴═H, R⁵═H).

This compound was prepared according to General Method 6 (EXAMPLE 141) from Compound 155 (EXAMPLE 139) to afford Compound 163. ¹H NMR (500 MHz, CDCl₃) δ 8.21 (d, J=8.1 Hz, 1H), 8.17 (d, J=8.7 Hz, 1H), 7.39 (t, J=8.1 Hz, 1H), 7.24 (m, 1H), 6.81 (d, J=8.6 Hz, 1H), 6.79 (d, J=8.6 Hz, 1H), 6.70 (d, J=8.7 Hz, 1H), 5.94 (s, 1H), 5.56 (m, 1H), 5.52 (s, 1H), 4.69 (d, J=5.7 Hz, 2H), 4.21 (s, 1H), 3.80 (s, 3H), 2.14 (d, J=1.3 Hz, 3H), 1.36 (s, 6H).

### Example 148

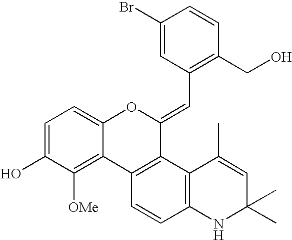

(Z)-5-(5′-Bromo-2′-hydroxymethylbenzylidene)1,2-dihydro-9-hydroxy-10-methoxy-2,2,4-trimethyl-5H-chromeno[3,4-f]quinoline (Compound 164, Structure 37 of Scheme X, where R³═H, R⁴═H, R⁵═Br).

This compound was prepared according to General Method 6 (EXAMPLE 141) from Compound 91 (EXAMPLE 76) to afford Compound 164. ¹H NMR (500 MHz, CDCl₃) δ 8.42 (d, J=2.1 Hz, 1H), 8.19 (d, J=8.5 Hz, 1H), 7.34 (dd, J=8.1, 2.1 Hz, 1H), 7.26 (d, J=8.1 Hz, 1H), 6.86 (d, J=8.7 Hz, 1H), 6.83 (d, J=8.7 Hz, 1H), 6.71 (d, J=8.5 Hz, 1H), 5.85 (s, 1H), 5.57 (s, 1H), 5.52 (q, J=1.2 Hz, 1H), 4.63 (s, 2H), 3.80 (s, 3H), 2.11 (d, J=1.2 Hz, 3H), 1.35 (s, 6H).

### Example 149

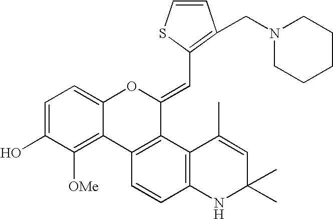

(Z)-5-(2′-(3′-(Piperidinylmethyl)thienylmethylidene))1,2-dihydro-9-hydroxy-10-methoxy-2,2,4-trimethyl-5H-chromeno[3,4-f]quinoline (Compound 165, Structure 10 of Scheme III, where X═S, R¹³═H, R¹⁴R¹⁵=—(CH₂)₅—).

General Method 7: Reduction of a tertiary amide to the corresponding amine. The product is light sensitive. 0.5 M Alane-N,N-dimethylethylamine complex in toluene (5 equiv.) was added dropwise to a solution of amide (1 equiv.) at 0° C. The solution was allowed to warm to room temperature and stirred for 1 h. Methanol (25 mL/mmol), acetic acid (1.8 mmol) and sodium cyanoborohydride (12 equiv.) were added sequentially and the solution stirred at room temperature for 0.2 h. The reaction was concentrated under reduced pressure, ethyl acetate (100 mL/mmol) added, the solution washed with a saturated solution of sodium hydrogen carbonate (100 ml/mmol), a saturated solution of ammonium chloride (100 mL/mmol), dried (Na₂SO₄) and concentrated under reduced pressure. Purification by flash chromatography, eluting with ethyl acetate:hexanes afforded the tertiary amine.

(Z)-5-(2′-(3′-(Piperidinylmethyl)thienylmethylidene))1,2-dihydro-9-hydroxy-10-methoxy-2,2,4-trimethyl-5H-chromeno[3,4-f]quinoline (Compound 165, Structure 10 of Scheme III, where X═S, R¹³═H, R¹⁴R¹⁵=—(CH₂)₅—) was prepared according to General Method 7 (EXAMPLE 149) from Compound 140 (EXAMPLE 124) to afford Compound 165. ¹H NMR (500 MHz, Acetone-d₆) δ 8.30 (d, J=8.5 Hz, 1H), 7.74 (s, 1H), 7.29 (d, J=5.1 Hz, 1H), 6.99 (d, J=8.5 Hz, 1H), 6.92 (d, J=5.1 Hz, 1H), 6.80 (d, J=8.7 Hz, 1H), 6.79 (d, J=8.7 Hz, 1H), 6.35 (s, 1H), 5.89 (br s, 1H), 5.54 (m, 1H), 3.76 (s, 3H), 3.38 (s, 2H), 2.33 (m, 4H), 2.11 (d, J=1.2 Hz, 3H), 1.53 (m, 4H), 1.41 (s, 2H), 1.34 (m, 6H).

### Example 150

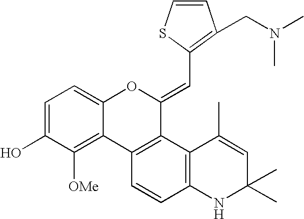

(Z)-5-(2′-(3′-(Dimethylaminomethyl)thienylmethylidene))1,2-dihydro-9-hydroxy-10-methoxy-2,2,4-trimethyl-5H-chromeno[3,4-f]quinoline (Compound 166, Structure 10 of Scheme III, where X═S, R¹³═H, R¹⁹,R²⁰=Me).

This compound was prepared according to General Method 7 (EXAMPLE 149) from Compound 132 (EXAMPLE 117) to afford Compound 166. ¹H NMR (500 MHz, Acetone-d₆) δ 8.30 (d, J=8.7 Hz, 1H), 7.77 (s, 1H), 7.30 (dd, J=5.2, 0.6 Hz, 1H), 6.98 (d, J=8.7 Hz, 1H), 6.93 (d, J=5.2 Hz, 1H), 6.80 (d, J=8.7 Hz, 1H), 6.79 (d, J=8.7 Hz, 1H), 6.45 (d, J=0.6 Hz, 1H), 5.87 (s, 1H), 5.53 (q, J=1.4 Hz, 1H), 3.76 (s, 3H), 3.39 (s, 2H), 2.15 (s, 6H), 2.08 (d, J=1.4 Hz, 3H), 1.34 (s, 6H).

### Example 151

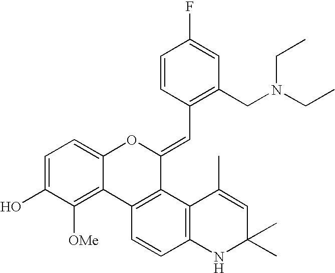

(Z)-5-(2′-(Diethylaminomethyl)-4′-fluorobenzylidene)1,2-dihydro-9-hydroxy-10-methoxy-2,2,4-trimethyl-5H-chromeno[3,4-f]quinoline (Compound 167, Structure 39 of Scheme X, where R³═H, R⁴═F, R⁵═H).

This compound was prepared according to General Method 7 (EXAMPLE 149) from Compound 83 (EXAMPLE 68) to afford Compound 167. ¹H NMR (500 MHz, CDCl₃) δ 8.16 (d, J=8.6 Hz, 1H), 8.15 (d, J=8.5 Hz, 1H), 7.13 (dd, J=10.0, 2.8 Hz, 1H), 6.98 (td, J=8.6, 2.8 Hz, 1H), 6.79 (d, J=8.8 Hz, 1H), 6.77 (d, J=8.8 Hz, 1H), 6.67 (d, J=8.5 Hz, 1H), 6.09 (s, 1H), 5.48 (q, J=1.2 Hz, 1H), 4.18 (s, 1H), 3.79 (s, 3H), 3.48 (s, 2H), 2.48 (q, J=7.1 Hz, 4H), 2.13 (d, J=1.2 Hz, 3H), 1.38 (broad s, 6H), 0.94 (t, J=7.1 Hz, 6H).

### Example 152

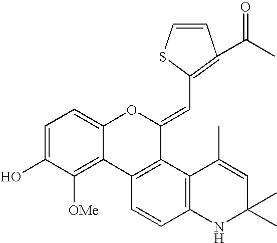

(Z)-5-(2′-(3′-Acetylthienylmethylidene))1,2-dihydro-9-hydroxy-10-methoxy-2,2,4-trimethyl-5H-chromeno[3,4-f]quinoline (Compound 168, Structure 6 of Scheme III, where X═S, R¹³═H, R^{A}=Me).

General Method 8: Addition of an organometallic reagent to N,N-dialkylated amide to generate a ketone. The product is light sensitive. A flame-dried round bottom flask was charged with Compound 140 (EXAMPLE 124) (1 equiv.), and 1:1 (v/v) tetrahydrofuran and diethyl ether (10 mL/mmol). The resulting slurry was cooled to 0° C. and the organolithium (10 equiv., 1.6 M in diethyl ether) was added dropwise over 0.3 h. The reaction was allowed to warm to ambient temperature over 5 h and quenched with an aqueous solution of saturated ammonium chloride (10 mL/mmol). The aqueous layer was extracted with ethyl acetate (2×5 mL/mmol). The combined organics were washed with brine (15 mL/mmol), dried over sodium sulfate, filtered, and concentrated in vacuo. The crude product was purified by silica gel chromatography using dichloromethane as eluent to provide the title compound as an orange solid.

General Method 8A: The procedure is similar to General Method 8 except that a ketone or aldehyde is used in place of the amide, and an organomagnesium reagent (2-3 equiv.) can be substituted for an organolithium reagent (2-3 equiv.). These reactions can be conducted in either THF or diethyl ether.

(Z)-5-(2′-(3′-Acetylthienylmethylidene))1,2-dihydro-9-hydroxy-10-methoxy-2,2,4-trimethyl-5H-chromeno[3,4-f]quinoline (Compound 168, Structure 6 of Scheme III, where X═S, R¹³═H, R^{A}=Me) was prepared according to General Method 8 (EXAMPLE 152) from Compound 140 (EXAMPLE 124) and methyllithium (1.6 M in diethyl ether) to afford Compound 168, mp 222-224° C. ¹H NMR (500 MHz, CD₃OD) δ 8.36 (d, J=8.8 Hz, 1H), 7.50 (d, J=5.6 Hz, 1H), 7.42 (d, J=0.6 Hz, 1H), 7.29 (dd, J=5.6, 0.6 Hz, 1H), 6.99 (d, J=8.8 Hz, 1H), 6.79 (d, J=8.8 Hz, 1H), 6.76 (d, J=8.8 Hz, 1H), 5.53 (q, J=1.2 Hz, 1H), 3.76 (s, 3H), 2.52 (s, 3H), 2.01 (d, J=1.2 Hz, 3H), 1.33 (s, 6H).

### Example 153

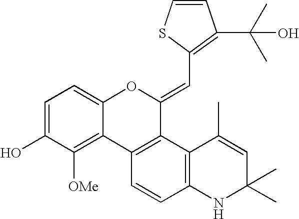

(Z)-5-(2′-(3′-(1″-Hydroxy-1″-methylethyl)thienylmethylidene))1,2-dihydro-9-hydroxy-10-methoxy-2,2,4-trimethyl-5H-chromeno[3,4-f]quinoline (Compound 169, Structure 8 of Scheme III, where X═S, R¹³═H, R^{A},R^{B}=Me).

This compound was prepared according to General Method 8A (EXAMPLE 152) from Compound 168 and methyllithium (1.6 M in diethyl ether) to afford Compound 169. ¹H NMR (500 MHz, CD₃OD) δ 8.28 (d, J=8.7 Hz, 1H), 7.19 (dd, J=5.4, 0.7 Hz, 1H), 7.04 (d, J=5.4 Hz, 1H), 6.92 (d, J=8.7 Hz, 1H), 6.73 (d, J=8.7 Hz, 1H), 6.71 (d, J=8.7 Hz, 1H), 6.66 (d, J=0.7 Hz, 1H), 5.49 (q, J=1.2 Hz, 1H), 3.75 (s, 3H), 2.04 (d, J=1.2 Hz, 3H), 1.55 (s, 6H), 1.30 (s, 6H).

### Example 154

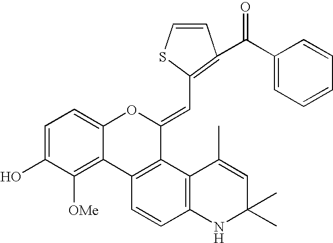

(Z)-5-(2′-(3′-Benzoylthienylmethylidene))1,2-dihydro-9-hydroxy-10-methoxy-2,2,4-trimethyl-5H-chromeno[3,4-f]quinoline (Compound 170, Structure 6 of Scheme III, where X═S, R¹³═H, R^{A}=Ph).

This compound was prepared according to General Method 8 (EXAMPLE 152) from Compound 140 (EXAMPLE 124) and phenyllithium to afford Compound 170. ¹H NMR (500 MHz, CDCl₃) δ 8.18 (d, J=8.5 Hz, 1H), 7.76 (dd, J=7.8, 1.3 Hz, 2H), 7.53 (tt, J=7.8, 1.3 Hz, 1H), 7.43 (t, J=7.8 Hz, 2H), 7.19 (dd, J=5.4, 0.7 Hz, 1H), 7.12 (d, J=5.4 Hz, 1H), 7.09 (d, J=8.8 Hz, 1H), 6.87 (d, J=8.8 Hz, 1H), 6.81 (d, J=0.7 Hz, 1H), 6.67 (d, J=8.5 Hz, 1H), 5.62 (s, 1H), 5.54 (q, J=1.3 Hz, 1H), 3.77 (s, 3H), 2.09 (d, J=1.3 Hz, 3H), 1.29 (s, 6H).

### Example 155

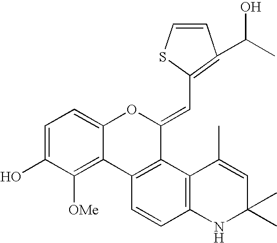

(±)-(Z)-5-(2′-(3′-(1″-Hydroxyethyl)thienylmethylidene))1,2-dihydro-9-hydroxy-10-methoxy-2,2,4-trimethyl-5H-chromeno[3,4-f]quinoline (Compound 171, Structure 11 of Scheme III, where X═S, R¹³═H, R^{A}=Me).

General Method 9. Reduction of a ketone to an alcohol. A round-bottom flask was charged with the ketone (35 mg, 0.076 mmol) and 20-40 mL/mmol of dry methanol. The flask was cooled to 0° C. and sodium borohydride (2.1 equiv.) was added as a white solid in a single portion. The reaction was stirred for 0.5 h then poured into water (10 mL). The aqueous phase was extracted with ethyl acetate (3×10 mL) and the combined organics were washed with brine (1×30 mL), dried over sodium sulfate, and concentrated. Purification by silica gel column chromatography (2/1; hexanes/ethyl acetate), afforded the desired alcohol.

(±)-(Z)-5-(2′-(3′-(1″-Hydroxyethyl)thienylmethylidene))1,2-dihydro-9-hydroxy-10-methoxy-2,2,4-trimethyl-5H-chromeno[3,4-f]quinoline (Compound 171, Structure 11 of Scheme III, where X═S, R¹³═H, R^{A}=Me) was prepared according to General Method 9 (EXAMPLE 155) from Compound 168 to afford Compound 171. ¹H NMR (500 MHz, CD₃OD) δ 8.30 (d, J=8.8 Hz, 1H), 7.27 (d, J=5.3 Hz, 1H), 7.10 (d, J=5.3 Hz, 1H), 6.93 (d, J=8.8 Hz, 1H), 6.74 (d, J=8.8 Hz, 1H), 6.73 (d, J=8.8 Hz, 1H), 6.10 (s, 1H), 5.52 (q, J=1.2 Hz, 1H), 4.97 (q, J=6.4 Hz, 1H), 3.75 (s, 3H), 2.05 (d, J=1.2 Hz, 3H), 1.42 (d, J=6.4 Hz, 3H), 1.30 (s, 6H).

### Example 156

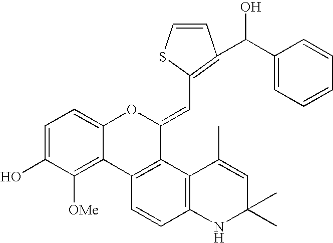

(±)-(Z)-5-(2′-(3′-(1″-Hydroxy-1″-phenylmethyl)thienylmethylidene))1,2-dihydro-9-hydroxy-10-methoxy-2,2,4-trimethyl-5H-chromeno[3,4-f]quinoline (Compound 172, Structure 11 of Scheme III, where X═S, R¹³═H, R^{A}=Ph).

This compound was prepared according to General Method 9 (EXAMPLE 155) from Compound 170 (EXAMPLE 154) to afford Compound 172. ¹H NMR (500 MHz, CDCl₃) δ 8.16 (d, J=8.5 Hz, 1H), 7.36 (d, J=7.5 Hz, 2H), 7.31 (t, J=7.5 Hz, 2H), 7.27 (m, 1H), 7.23 (d, J=5.4 Hz, 1H), 7.07 (d, J=5.4 Hz, 1H), 7.04 (d, J=8.8 Hz, 1H), 6.83 (d, J=8.8 Hz, 1H), 6.66 (d, J=8.5 Hz, 1H), 6.13 (s, 1H), 5.99 (q, J=1.5 Hz, 1H), 5.57 (s, 1H), 5.47 (s, 1H), 4.19 (s, 1H), 3.76 (s, 3H), 2.14 (br, 1H), 1.89 (m, 3H), 1.36 (s, 6H)

### Example 157

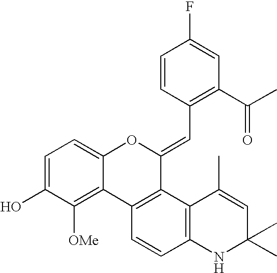

(Z)-5-(4′-Fluoro-2′-acetylbenzylidene)1,2-dihydro-9-hydroxy-10-methoxy-2,2,4-trimethyl-5H-chromeno[3,4-f]quinoline (Compound 173, Structure 35 of Scheme X, where R³═H, R⁴═F, R⁵═H, R^{A}=Me).

This compound was prepared according to General Method 8 (EXAMPLE 152) from Compound 94 (EXAMPLE 79) to afford Compound 173 in 21% yield. ¹H NMR (500 MHz, CDCl₃) δ 8.17 (d, J=8.5 Hz, 1H), 8.17 (m, 1H), 7.28 (dd, J=9.0, 2.7 Hz, 1H), 7.21 (td, J=9.0, 2.7 Hz, 1H), 6.78 (d, J=8.8 Hz, 1H), 6.75 (d, J=8.8 Hz, 1H), 6.69 (d, J=8.5 Hz, 1H), 6.14 (s, 1H), 5.59 (br s, 1H), 5.53 (q, J=1.2 Hz, 1H), 4.21 (s, 1H), 3.78 (s, 3H), 2.49 (s, 3H), 2.10 (d, J=1.2 Hz, 3H), 1.35 (s, 6H).

### Example 158

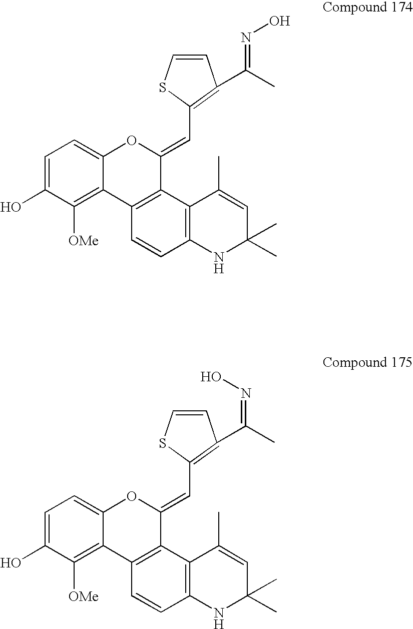

(Z)-5-(2′-(3′-((E)-1″-Hydroxyiminoethyl)thienylmethylidene))1,2-dihydro-9-hydroxy-10-methoxy-2,2,4-trimethyl-5H-chromeno[3,4-f]quinoline (Compound 174, Structure 7 of Scheme III, where X═S, R¹³═H, R^{A}=Me, R^{C}=(E)-OH) and (Z)-5-(2′-(3′-((Z)-1″-Hydroxyiminoethyl)thienylmethylidene))1,2-dihydro-9-hydroxy-10-methoxy-2,2,4-trimethyl-5H-chromeno[3,4-f]quinoline (Compound 175, Structure 7 of Scheme III, where X═S, R¹³═H, R^{A}=Me, R^{C}=(Z)-OH).

General Method 10: Preparation of an oxime derived from a ketone or aldehyde. The product is light sensitive. Into a flame-dried round bottom flask equipped with a magnetic stir bar was added the carbonyl compound (1 equiv), absolute ethyl alcohol (10 mL/mmol), and the hydroxyl- or alkoxyamine hydrochloride (2.5 equiv). The reaction was warmed to 40° C. for 1.5 h, then concentrated in vacuo to a white solid. If the reaction is not completed, then heating should be continued until conversion is satisfactory. The resulting solid was taken up in ethyl acetate/water (1:1, 30 mL/mmol). The aqueous phase was extracted with ethyl acetate (2×10 mL/mmol) and the combined organics washed with brine and dried over sodium sulfate. The residue was purified by silica gel chromatography using dichloromethane as eluent to provide the desired oxime. Alternatively, the compounds can be isolated by HPLC (chromasil C-18, eluted with MeOH:water).

(Z)-5-(2′-(3′-((E)-1″-Hydroxyiminoethyl)thienylmethylidene))1,2-dihydro-9-hydroxy-10-methoxy-2,2,4-trimethyl-5H-chromeno[3,4-f]quinoline (Compound 174, Structure 7 of Scheme III, where X═S, R¹³═H, R^{A}=Me, R^{C}=(E)-OH) and (Z)-5-(2′-(3′-((Z)-1″-Hydroxyiminoethyl)thienylmethylidene))1,2-dihydro-9-hydroxy-10-methoxy-2,2,4-trimethyl-5H-chromeno[3,4-f]quinoline (Compound 175, Structure 7 of Scheme III, where X═S, R¹³═H, R^{A}=Me, R^{C}=(Z)-OH). These compounds were prepared according to General Method 10 (EXAMPLE 158) from Compound 168 (EXAMPLE 152) and hydroxylamine hydrochloride to afford Compound 174 and Compound 175. Compound 174: ¹H NMR (500 MHz, CD₃OD) δ 8.30 (d, J=8.8 Hz, 1H), 7.35 (dd, J=5.3, 0.8 Hz, 1H), 6.95 (d, J=8.8 Hz, 1H), 6.89 (d, J=5.3 Hz, 1H), 6.73 (d, J=8.8 Hz, 2H), 5.86 (d, J=0.8 Hz, 1H), 5.49 (q, J=1.2 Hz, 1H), 3.75 (s, 3H), 2.06 (s, 3H), 2.02 (d, J=1.2 Hz, 3H), 1.28 (s, 6H). Compound 175: ¹H NMR (500 MHz, CD₃OD) δ 8.30 (d, J=8.8 Hz, 1H), 7.31 (dd, J=5.4, 0.7 Hz, 1H), 7.04 (d, J=5.4 Hz, 1H), 6.94 (d, J=8.8 Hz, 1H), 6.73 (d, J=8.8 Hz, 1H), 6.73 (d, J=8.8 Hz, 1H), 6.30 (d, J=0.7 Hz, 1H), 5.52 (q, J=1.2 Hz, 1H), 3.75 (s, 3H), 2.15 (s, 3H), 2.01 (d, J=1.2 Hz, 3H), 1.29 (s, 6H).

### Example 159

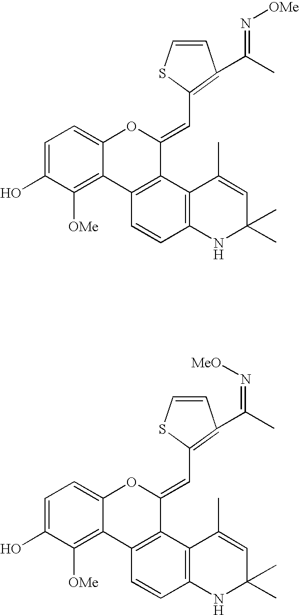

(Z)-5-(2′-(3′-((E)-1″-Methoxyiminoethyl)thienylmethylidene))1,2-dihydro-9-hydroxy-10-methoxy-2,2,4-trimethyl-5H-chromeno[3,4-f]quinoline (Compound 176, Structure 7 of Scheme III, where X═S, R¹³═H, R^{A}=Me, R^{C}=(E)-OMe), and (Z)-5-(2′-(3′-((Z)-1″-Methoxyiminoethyl)thienylmethylidene))1,2-dihydro-9-hydroxy-10-methoxy-2,2,4-trimethyl-5H-chromeno[3,4-f]quinoline (Compound 177, Structure 7 of Scheme III, where X═S, R¹³═H, R^{A}=Me, R^{C}=(Z)-OMe).

These compounds were prepared according to General Method 10 (EXAMPLE 158) from Compound 168 (EXAMPLE 152) and methoxyamine hydrochloride to afford Compound 176 and Compound 177. Data for Compound 176: ¹H NMR (500 MHz, CD₃OD) δ 8.30 (d, J=8.8 Hz, 1H), 7.32 (dd, J=5.4, 0.8 Hz, 1H), 7.08 (d, J=5.4 Hz, 1H), 6.97 (d, J=8.8 Hz, 1H), 6.74 (d, J=8.8 Hz, 1H), 6.73 (d, J=8.8 Hz, 1H), 6.55 (d, J=0.8 Hz, 1H), 5.49 (q, J=1.2 Hz, 1H), 3.92 (s, 3H), 3.75 (s, 3H), 2.15 (s, 3H), 2.06 (d, J=1.2 Hz, 3H), 1.29 (s, 6H). Data for Compound 177: ¹H NMR (500 MHz, CD₃OD) δ 8.30 (d, J=8.6 Hz, 1H), 7.35 (dd, J=5.3, 1.0 Hz, 1H), 6.94 (d, J=8.6 Hz, 1H), 6.86 (d, J=5.3 Hz, 1H), 6.74 (d, J=8.6 Hz, 1H), 6.73 (d, J=8.6 Hz, 1H), 5.80 (d, J=1.0 Hz, 1H), 5.48 (q, J=1.2 Hz, 1H), 3.75 (s, 3H), 3.73 (s, 3H), 2.05 (s, 3H), 2.03 (d, J=1.2 Hz, 3H), 1.29 (s, 6H).

### Example 160

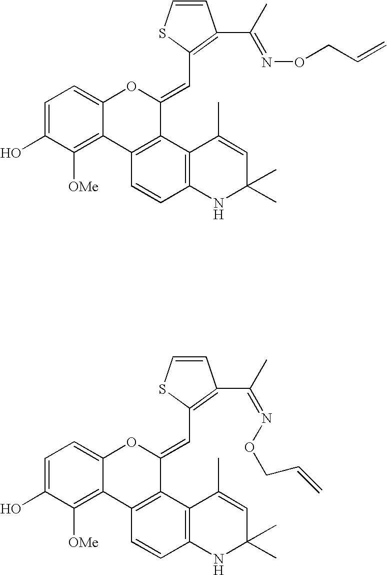

(Z)-5-(2′-(3′-((E)-1″-Allyloxyiminoethyl)thienylmethylidene))1,2-dihydro-9-hydroxy-10-methoxy-2,2,4-trimethyl-5H-chromeno[3,4-f]quinoline (Compound 178, Structure 7 of Scheme III, where X═S, R¹³═H, R^{A}=Me, R^{C}=(E)-O-allyl) and (Z)-5-(2′-(3′-((Z)-1″-Allyloxyiminoethyl)thienylmethylidene))1,2-dihydro-9-hydroxy-10-methoxy-2,2,4-trimethyl-5H-chromeno[3,4-f]quinoline (Compound 179, Structure 7 of Scheme III, where X═S, R¹³═H, R^{A}=Me, R^{C}=(Z)-O-allyl).

These compounds were prepared according to General Method 10 (EXAMPLE 158) from Compound 168 (EXAMPLE 152) and O-allylhydroxylamine hydrochloride to afford Compound 178 and Compound 179. Data for Compound 178: ¹H NMR (500 MHz, CD₃OD) δ 8.31 (d, J=8.7 Hz, 1H), 7.32 (dd, J=5.4, 0.6 Hz, 1H), 7.09 (d, J=5.4 Hz, 1H), 6.98 (d, J=8.7 Hz, 1H), 6.74 (m, 2H), 6.56 (d, J=0.6 Hz, 1H), 6.04 (ddt, J=17.1, 10.5, 5.6 Hz, 1H), 5.48 (m, 1H), 5.30 (dm, J=17.1 Hz, 1H), 5.19 (dm, J=10.5 Hz, 1H), 4.66 (dm, J=5.6 Hz, 2H), 3.75 (s, 3H), 2.19 (s, 3H), 2.06 (m, 3H), 1.29 (s, 6H). Data for Compound 179: ¹H NMR (500 MHz, CD₃OD) δ 8.31 (d, J=8.7 Hz, 1H), 7.36 (d, J=5.3 Hz, 1H), 6.94 (d, J=8.7 Hz, 1H), 6.88 (d, J=5.3 Hz, 1H), 6.75 (d, J=8.7 Hz, 1H), 6.74 (d, J=8.7 Hz, 1H), 5.83 (ddt, J=17.2, 10.5, 5.5 Hz, 1H), 5.81 (s, 1H), 5.48 (m, 1H), 5.09 (dm, J=17.2 Hz, 1H), 5.00 (dm, J=10.5 Hz, 1H), 4.43 (dt, J=5.5, 1.1 Hz, 2H), 3.76 (s, 3H), 2.07 (d, J=1.0 Hz, 3H), 2.02 (m, 3H), 1.29 (s, 6H).

### Example 161

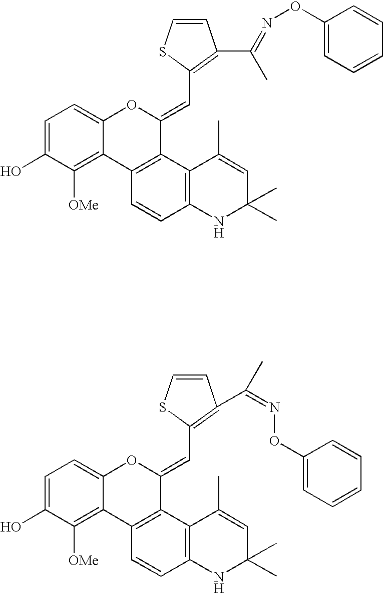

(Z)-5-(2′-(3′-((E)-1″-Phenoxyiminoethyl)thienylmethylidene))1,2-dihydro-9-hydroxy-10-methoxy-2,2,4-trimethyl-5H-chromeno[3,4-f]quinoline (Compound 180, Structure 7 of Scheme III, where X═S, R¹³═H, R¹=Me, R^{C}=(E)-O-phenyl), and (Z)-5-(2′-(3′-((Z)-1″-Phenoxyiminoethyl)thienylmethylidene))1,2-dihydro-9-hydroxy-10-methoxy-2,2,4-trimethyl-5H-chromeno[3,4-f]quinoline (Compound 181, Structure 7 of Scheme E11, where X═S, R¹³═H, R^{A}=Me, R^{C}=(Z)-O-phenyl).

These compounds were prepared according to General Method 10 (EXAMPLE 158) from Compound 168 (EXAMPLE 152) and O-phenylhydroxylamine hydrochloride to afford Compound 180 and Compound 181. Data for Compound 180: ¹H NMR (300 MHz, CD₃OD) δ 8.28 (d, J=8.7 Hz, 1H), 7.39 (m, 1H), 7.31 (m, 2H), 7.21-7.14 (m, 3H), 7.03 (m, 1H), 6.98 (d, J=8.7 Hz, 1H), 6.74 (d, J=8.7 Hz, 1H), 6.68 (d, J=8.7 Hz, 1H), 6.47 (m, 1H), 4.78 (m, 1H), 3.75 (s, 3H), 2.37 (s, 3H), 1.96 (m, 3H), 0.93 (s, 6H). Compound for 181: ¹H NMR (300 MHz, CD₃OD) δ 8.27 (d, J=8.7 Hz, 1H), 7.43 (m, 1H), 7.25-7.17 (m, 2H), 7.07-7.01 (m, 2H), 6.99-6.92 (m, 3H), 6.76-6.66 (m, 2H), 5.82 (m, 1H), 4.63 (m, 1H), 3.75 (s, 3H), 2.20 (s, 3H), 1.85 (m, 3H), 1.11 (s, 6H).

### Example 162

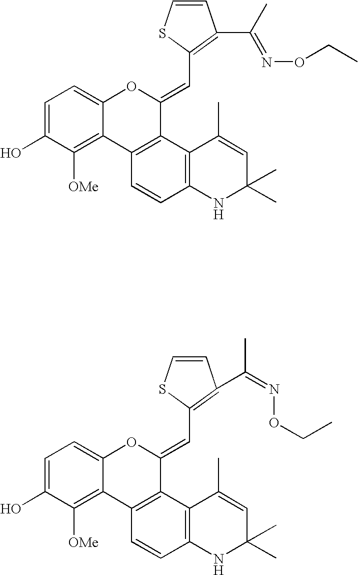

(Z)-5-(2′-(3′-((E)-1″-Ethoxyiminoethyl)thienylmethylidene))1,2-dihydro-9-hydroxy-10-methoxy-2,2,4-trimethyl-5H-chromeno[3,4-f]quinoline (Compound 182, Structure 7 of Scheme I, where X═S, R¹³═H, R^{A}=Me, R^{C}=(E)-OEt) and (Z)-5-(2′-(3′-((Z)-1″-Ethoxyiminoethyl)thienylmethylidene))1,2-dihydro-9-hydroxy-10-methoxy-2,2,4-trimethyl-5H-chromeno[3,4-f]quinoline (Compound 183, Structure 7 of Scheme III, where X═S, R¹³═H, R^{A}=Me, R^{C}=(Z)-OEt).

These compounds were prepared according to General Method 10 (EXAMPLE 158) from Compound 168 (EXAMPLE 152) and ethoxyamine hydrochloride to afford Compound 182 and Compound 183. Data for Compound 182: ¹H NMR (500 MHz, CD₃OD) δ 8.30 (d, J=8.7 Hz, 1H), 7.32 (dd, J=5.4, 0.8 Hz, 1H), 7.09 (d, J=5.4 Hz, 1H), 6.98 (d, J=8.7 Hz, 1H), 6.74 (d, J=8.7 Hz, 1H), 6.74 (d, J=8.7 Hz, 1H), 6.54 (d, J=0.8 Hz, 1H), 5.47 (q, J=1.3 Hz, 1H), 4.18 (q, J=7.1 Hz, 2H), 3.75 (s, 3H), 2.16 (s, 3H), 2.05 (d, J=1.3 Hz, 3H), 1.27 (t, J=7.1 Hz, 3H), 1.29 (s, 6H). Data for Compound 183: ¹H NMR (500 MHz, CD₃OD) δ 8.31 (d, J=8.7 Hz, 1H), 7.35 (dd, J=5.3, 0.6 Hz, 1H), 6.94 (d, J=8.7 Hz, 1H), 6.87 (d, J=5.3 Hz, 1H), 6.75 (d, J=8.7 Hz, 1H), 6.74 (d, J=8.7 Hz, 1H), 5.80 (d, J=0.6 Hz, 1H), 5.47 (q, J=1.1 Hz, 1H), 3.97 (q, J=7.0 Hz, 2H), 3.76 (s, 3H), 2.07 (s, 3H), 2.02 (m, 3H), 1.29 (s, 6H), 1.09 (t, J=7.0 Hz, 3H).

### Example 163

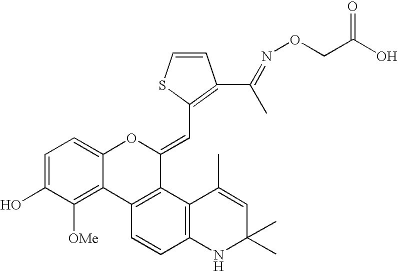

(Z)-5-(2′-(3′-((E)-(Carboxymethoxy)iminoethyl)thienylmethylidene))1,2-dihydro-9-hydroxy-10-methoxy-2,2,4-trimethyl-5H-chromeno[3,4-f]quinoline (Compound 184, Structure 7 of Scheme III, where X═S, R³═H, R^{A}=Me, R^{C}=(E)-OCH₂CO₂H).

This compound was prepared according to General Method 10 (EXAMPLE 158) from Compound 168 (EXAMPLE 152) and carboxymethoxylamine hemihydrochloride to afford Compound 184. ¹H NMR (500 MHz, CD₃OD) δ 8.29 (d, J=8.6 Hz, 1H), 7.30 (d, J=5.2 Hz, 1H), 7.11 (d, J=5.2 Hz, 1H), 6.97 (d, J=8.6 Hz, 1H), 6.73 (d, J=8.6 Hz, 1H), 6.73 (d, J=8.6 Hz, 1H), 6.52 (s, 1H), 5.63 (s, 1H), 3.75 (s, 3H), 2.26 (s, 3H), 2.05 (s, 3H), 1.32 (s, 6H).

### Example 164

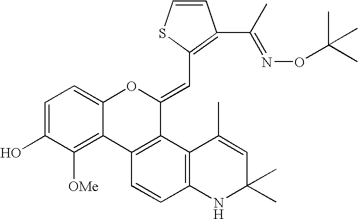

(Z)-5-(2′-(3′-((E)-1″-tert-Butoxyiminoethyl)thienylmethylidene))1,2-dihydro-9-hydroxy-10-methoxy-2,2,4-trimethyl-5H-chromeno[3,4-f]quinoline (Compound 185, Structure 7 of Scheme III, where X═S, R¹³═H, R^{A}=Me, R^{C}=(E)-O-tert-Bu).

This compound was prepared according to General Method 10 (EXAMPLE 158) from Compound 168 (EXAMPLE 152) and O-(tert-butyl)hydroxylamine hydrochloride to afford Compound 185. ¹H NMR (300 MHz, CD₃OD) δ 8.27 (d, J=8.6 Hz, 1H), 7.32 (d, J=5.3 Hz, 1H), 7.04 (d, J=5.3 Hz, 1H), 6.96 (d, J=8.6 Hz, 1H), 6.73 (d, J=8.6 Hz, 1H), 6.73 (d, J=8.6 Hz, 1H), 6.25 (s, 1H), 5.44 (m, 1H), 3.75 (s, 3H), 2.12 (s, 3H), 2.02 (s, 3H), 1.27 (s, 15H).

### Example 165

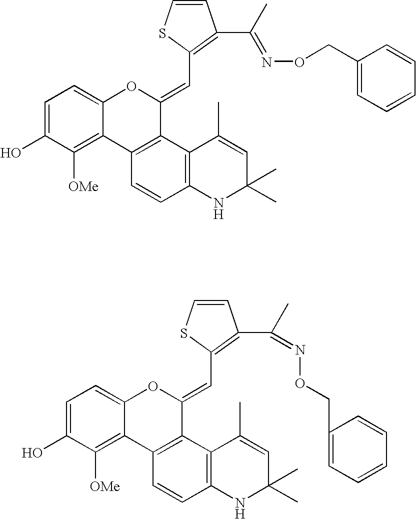

(Z)-5-(2′-(3′-((E)-1″-Benzyloxyiminoethyl)thienylmethylidene))1,2-dihydro-9-hydroxy-10-methoxy-2,2,4-trimethyl-5H-chromeno[3,4-f]quinoline (Compound 186, Structure 7 of Scheme III, where X═S, R³═H, R^{A}=Me, R^{C}=(E)-OBn), and (Z)-5-(2′-(3′-((Z)-1″-Benzyloxyiminoethyl)thienylmethylidene))1,2-dihydro-9-hydroxy-10-methoxy-2,2,4-trimethyl-5H-chromeno[3,4-f]quinoline (Compound 187, Structure 7 of Scheme III, where X═S, R³═H, R^{A}=Me, R^{C}=(Z)-OBn).

These compounds were prepared according to General Method 10 (EXAMPLE 158) from Compound 168 (EXAMPLE 152) to afford Compound 186 and Compound 187. Data for Compound 186: ¹H NMR (500 MHz, CD₃OD) δ 8.32 (d, J=8.7 Hz, 1H), 7.39 (m, 2H), 7.34 (m, 2H), 7.33 (dd, J=5.4, 0.6 Hz, 1H), 7.29 (tt, J=7.3, 1.4 Hz, 1H), 7.10 (d, J=5.4 Hz, 1H), 6.99 (d, J=8.7 Hz, 1H), 6.75 (d, J=8.7 Hz, 2H), 6.63 (d, J=0.6 Hz, 1H), 5.46 (q, J=1.2 Hz, 1H), 5.20 (s, 2H), 3.76 (s, 3H), 2.21 (s, 3H), 2.08 (d, J=1.2 Hz, 3H), 1.25 (s, 6H). Data for Compound 187: ¹H NMR (500 MHz, CD₃OD) δ 8.34 (d, J=8.7 Hz, 1H), 7.36 (dd, J=5.3, 0.7 Hz, 1H), 7.12-7.04 (m, 5H), 6.95 (d, J=8.7 Hz, 1H), 6.88 (d, J=5.3 Hz, 1H), 6.77 (d, J=8.7 Hz, 1H), 6.74 (d, J=8.7 Hz, 1H), 5.83 (d, J=0.7 Hz, 1H), 5.45 (q, J=1.2 Hz, 1H), 4.93 (s, 2H), 3.77 (s, 3H), 2.08 (s, 3H), 1.99 (d, J=1.2 Hz, 3H), 1.25 (s, 6H).

### Example 166

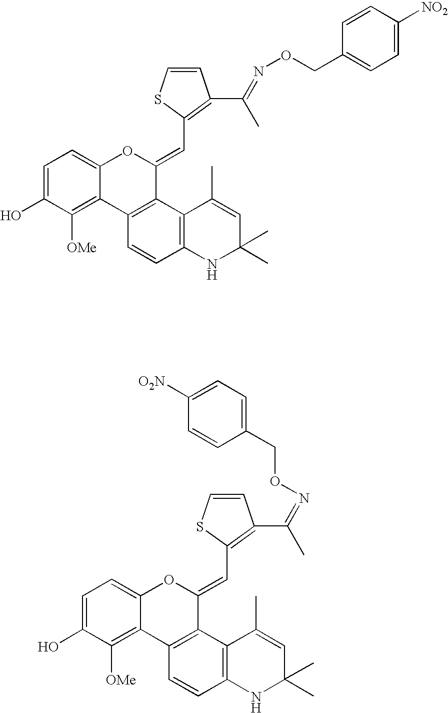

(Z)-5-(2′-(3′-((E)-1″-(p-Nitrobenzyloxy)iminoethyl)-thienylmethylidene))1,2-dihydro-9-hydroxy-10-methoxy-2,2,4-trimethyl-5H-chromeno[3,4-f]quinoline (Compound 188, Structure 7 of Scheme III, where X S, R¹³═H, R^{A}=Me, R^{C}=(E)-O-p-nitrobenzyl), and (Z)-5-(2′-(3′-((Z)-1″-(p-Nitrobenzyloxy)iminoethyl)thienylmethylidene))1,2-dihydro-9-hydroxy-10-methoxy-2,2,4-trimethyl-5H-chromeno[3,4-f]quinoline (Compound 189, Structure 7 of Scheme II, where X═S, R¹³═H, R^{A}=Me, R^{C}=(Z)-O-p-nitrobenzyl).

These compounds were prepared according to General Method 10 (EXAMPLE 158) from Compound 168 (EXAMPLE 152) to afford Compound 188 and Compound 189. Data for Compound 188: ¹H NMR (300 MHz, CD₃OD) δ 8.32 (d, J=8.7 Hz, 1H), 8.22 (d, J=8.8 Hz, 2H), 7.61 (d, J=8.8 Hz, 2H), 7.33 (d, J=5.4 Hz, 1H), 7.11 (d, J=5.4 Hz, 1H), 6.98 (d, J=8.7 Hz, 1H), 6.75 (d, J=8.7 Hz, 1H), 6.74 (d, J=8.7 Hz, 1H), 6.58 (s, 1H), 5.45 (m, 1H), 5.33 (s, 2H), 3.76 (s, 3H), 2.27 (s, 3H), 2.04 (m, 3H), 1.25 (s, 6H). Data for Compound 189: ¹H NMR (300 MHz, CD₃OD) δ 8.38 (d, J=8.8 Hz, 1H), 7.84 (d, J=8.6 Hz, 2H), 7.41 (d, J=5.4 Hz, 1H), 7.30 (d, J=8.6 Hz, 2H), 6.97 (d, J=8.7 Hz, 1H), 6.92 (d, J=5.4 Hz, 1H), 6.79 (d, J=8.7 Hz, 1H), 6.76 (d, J=8.8 Hz, 1H), 5.79 (s, 1H), 5.42 (s, 1H), 5.03 (s, 2H), 3.78 (s, 3H), 2.09 (s, 3H), 1.98 (m, 3H), 1.13 (s, 6H).

### Example 167

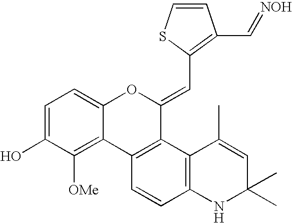

(Z)-5-(2′-(3′-((E)-Hydroxyiminomethyl)thienylmethylidene))1,2-dihydro-9-hydroxy-10-methoxy-2,2,4-trimethyl-5H-chromeno[3,4-f]quinoline (Compound 190, Structure 22 of Scheme V, where X═S, R¹³═H, R^{C}=(E)-OH).

(Z)-5-(2′-(3′-(Piperidinecarbonyl)thienylmethylidene))-1,2-dihydro-10-methoxy-2,2,4-trimethyl-9-(triisopropylsilyl)oxy-5H-chromeno[3,4-f]quinoline (Structure 12 of Scheme IV, where X═S, PG=triisopropylsilyl, R¹³═H, R¹⁴R¹⁵=—(CH₂)5-). This compound was prepared according to General Method 3 (EXAMPLE 135) from Compound 140 (EXAMPLE 124) to afford (Z)-5-(2′-(3′-(Piperidinecarbonyl)thienylmethylidene))-1,2-dihydro-10-methoxy-2,2,4-trimethyl-9-(triisopropylsilyl)oxy-5H-chromeno[3,4-f]quinoline.

(Z)-5-(2′-(3′-Hydroxymethylthienylmethylidene))-1,2-dihydro-10-methoxy-2,2,4-trimethyl-9-(triisopropylsilyl)oxy-5H-chromeno[3,4-f]quinoline (Structure 13 of Scheme IV, where X═S, PG=triisopropylsilyl, R¹³═H). This compound was prepared by General Method 6 (EXAMPLE 141) from (Z)-5-(2′-(3′-(piperidinecarbonyl)thienylmethylidene))-1,2-dihydro-10-methoxy-2,2,4-trimethyl-9-(triisopropylsilyl)oxy-5H-chromeno[3,4-f]quinoline to afford (Z)-5-(2′-(3′-hydroxymethylthienylmethylidene))-1,2-dihydro-10-methoxy-2,2,4-trimethyl-9-(triisopropylsilyl)oxy-5H-chromeno[3,4-f]quinoline.

(Z)-5-(2′-(3′-Formylthienylmethylidene))-1,2-dihydro-10-methoxy-2,2,4-trimethyl-9-(triisopropylsilyl)oxy-5H-chromeno[3,4-f]quinoline (Structure 18 of Scheme V, where X═S, PG=triisopropylsilyl, R¹³═H).

General Method 11: Oxidation of an alcohol to a ketone using IBX. 1-Hydroxy 1,2-benziodoxal-3(1H)-one-1-oxide (IBX) (3 equiv.) was added in one portion to a solution of the alcohol (1 equiv.) in 1:1 tetrahydrofuran:dimethylsulfoxide (30 mL/mmol) at 0° C. The suspension was allowed to warm to room temperature and stirred for 1 h. The reaction was poured into ice-water (100 mL/mmol) and extracted with ethyl acetate (3×20 mL/mmol). The combined organic extracts were washed with a saturated solution of ammonium chloride (60 mL/mmol), dried (Na₂SO₄) and concentrated under reduced pressure. Purification by flash chromatography, eluting with ethyl acetate:hexanes gave the corresponding aldehyde.

(Z)-5-(2′-(3′-Formylthienylmethylidene))-1,2-dihydro-10-methoxy-2,2,4-trimethyl-9-(triisopropylsilyl)oxy-5H-chromeno[3,4-f]quinoline (Structure 18 of Scheme V, where X═S, PG=triisopropylsilyl, R¹³═H). This compound was prepared according to General Method 11 from (Z)-5-(2′-(3′-hydroxymethylthienylmethylidene))-1,2-dihydro-10-methoxy-2,2,4-trimethyl-9-(triisopropylsilyl)oxy-5H-chromeno[3,4-f]quinoline to afford (Z)-5-(2′-(3′-formylthienylmethylidene))-1,2-dihydro-10-methoxy-2,2,4-trimethyl-9-(triisopropylsilyl)oxy-5H-chromeno[3,4-f]quinoline.

(Z)-5-(2′-(3′-((E)-Hydroxyiminomethyl)thienylmethylidene))-1,2-dihydro-10-methoxy-2,2,4-trimethyl-9-(triisopropylsilyl)oxy-5H-chromeno[3,4-f]quinoline. This compound was prepared according to General Method 10 (EXAMPLE 158) from (Z)-5-(2′-(3′-formylthienylmethylidene))-1,2-dihydro-10-methoxy-2,2,4-trimethyl-9-(triisopropylsilyl)oxy-5H-chromeno[3,4-f]quinoline to afford (Z)-5-(2′-(3′-((E)-hydroxyiminomethyl)thienylmethylidene))-1,2-dihydro-10-methoxy-2,2,4-trimethyl-9-(triisopropylsilyl)oxy-5H-chromeno[3,4-f]quinoline.

General Method 12: Deprotection of a triisopropylsilyl-protecting group with tetrabutylammonium fluoride (TBAF). Tetrabutylammonium fluoride (1.0 M solution in THF, 2.9 equiv.) was added dropwise to a solution of the silyl ether (1 equiv.) in tetrahydrofuran (10 mL) at 0° C. The reaction solution was stirred for 0.2 h at 0° C., then a saturated solution of ammonium chloride (10 mL) was added. The aqueous layer was extracted with ethyl acetate (3×10 mL), the combined organic extracts washed with a saturated solution of ammonium chloride, dried (Na₂SO₄) and concentrated under reduced pressure. Purification by flash chromatography, eluting with ethyl acetate:hexanes gave the desired phenol.

(Z)-5-(2′-(3′-((E)-Hydroxyiminomethyl)thienylmethylidene))1,2-dihydro-9-hydroxy-10-methoxy-2,2,4-trimethyl-5H-chromeno[3,4-f]quinoline (Compound 190, Structure 22 of Scheme V, where X═S, R¹³═H, R^{A}=Me, R^{C}=(E)-OH). This compound was prepared according to General Method 12 (EXAMPLE 167) from (Z)-5-(2′-(3′-((E)-hydroxyiminomethyl)thienylmethylidene))-1,2-dihydro-10-methoxy-2,2,4-trimethyl-9-(triisopropylsilyl)oxy-5H-chromeno[3,4-f]quinoline to afford Compound 190. ¹H NMR (500 MHz, Acetone-d₆) δ 10.19 (s, 1H), 8.33 (d, J=8.6 Hz, 1H), 8.24 (s, 1H), 7.82 (s, 1H), 7.40 (dd, J=5.3, 0.6 Hz, 1H), 7.30 (d, J=5.3 Hz, 1H), 7.01 (d, J=8.6 Hz, 1H), 6.83 (d, J=8.6 Hz, 1H), 6.82 (d, J=8.6 Hz, 1H), 6.39 (d, J=0.6 Hz, 1H), 5.94 (s, 1H), 5.58 (q, J=1.4 Hz, 1H), 3.78 (s, 3H), 2.06 (d, J=1.4 Hz, 3H), 1.35 (s, 6H).

### Example 168

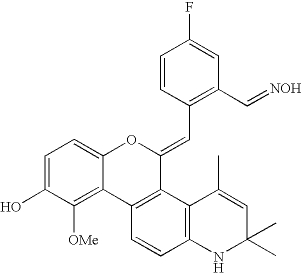

(Z)-5-(4′-Fluoro-(E)-2′-(hydroxyiminomethyl)benzylidene)1,2-dihydro-9-hydroxy-10-methoxy-2,2,4-trimethyl-5H-chromeno[3,4-f]quinoline (Compound 191, Structure 46 of Scheme XII, where R³═H, R⁴═F, R⁵═H, R^{C}═OH).

(Z)-5-(2′-(Pyrrolidinecarbonyl-4′-fluorobenzylidene)1,2-dihydro-10-methoxy-2,2,4-trimethyl-9-(triisopropylsilyl)oxy-5H-chromeno[3,4-f]quinoline (Structure 41 of Scheme XI, where R³═H, R¹═F, R⁵═H). This compound was prepared according to General Method 3 (EXAMPLE 135) from (Z)-5-(2′-(pyrrolidinecarbonyl-4′-fluorobenzylidene)1,2-dihydro-9-hydroxy-10-methoxy-2,2,4-trimethyl-5H-chromeno[3,4-f]quinoline (Compound 94, EXAMPLE 79) to afford (Z)-5-(2′-(pyrrolidinecarbonyl-4′-fluorobenzylidene)1,2-dihydro-10-methoxy-2,2,4-trimethyl-9-(triisopropylsilyl)oxy-5H-chromeno[3,4-f]quinoline.

(Z)-5-(4′-Fluoro-2′-hydroxymethylbenzylidene)1,2-dihydro-10-methoxy-2,2,4-trimethyl-9-(triisopropylsilyl)oxy-5H-chromeno[3,4-f]quinoline (Structure 42 of Scheme XI, where R³═H, R⁴═F, R⁵═H). This compound was prepared according to General Method 6 (EXAMPLE 141) from (Z)-5-(2′-(pyrrolidinecarbonyl-4′-fluorobenzylidene)1,2-dihydro-10-methoxy-2,2,4-trimethyl-9-(triisopropylsilyl)oxy-5H-chromeno[3,4-f]quinoline to afford (Z)-5-(4′-fluoro-2′-hydroxymethylbenzylidene)1,2-dihydro-10-methoxy-2,2,4-trimethyl-9-(triisopropylsilyl)oxy-5H-chromeno[3,4-f]quinoline.

(Z)-5-(4′-Fluoro-2′-formylbenzylidene)1,2-dihydro-10-methoxy-2,2,4-trimethyl-9-(triisopropylsilyl)oxy-5H-chromeno[3,4-f]quinoline (Structure 44 of Scheme XI, where R³═H, R⁴═F, R⁵═H). This compound was prepared according to General Method 11 (EXAMPLE 167) from (Z)-5-(4′-fluoro-2′-hydroxymethylbenzylidene)1,2-dihydro-10-methoxy-2,2,4-trimethyl-9-(triisopropylsilyl)oxy-5H-chromeno[3,4-f]quinoline to afford (Z)-5-(4′-fluoro-2′-formylbenzylidene)1,2-dihydro-10-methoxy-2,2,4-trimethyl-9-(triisopropylsilyl)oxy-5H-chromeno[3,4-f]quinoline.

(Z)-5-(4′-Fluoro-(E)-2′-(hydroxyiminomethyl)benzylidene)1,2-dihydro-9-hydroxy-10-methoxy-2,2,4-trimethyl-5H-chromeno[3,4-f]quinoline (Compound 191, Structure 46 of Scheme XII, where R³═H, R⁴═F, R⁵═H, R^{C}═OH). This compound was prepared by sequential treatment of (Z)-5-(4′-fluoro-2′-formylbenzylidene)1,2-dihydro-10-methoxy-2,2,4-trimethyl-9-(triisopropylsilyl)oxy-5H-chromeno[3,4-f]quinoline with General Method 12 (EXAMPLE 167) and General Method 10 (EXAMPLE 158) using hydroxylamine hydrochloride to afford Compound 191. ¹H NMR (500 MHz, CDCl₃) δ8.29 (d, J=1.5 Hz, 1H), 8.18 (d, J=8.5 Hz, 1H), 8.01 (dd, J=8.6, 5.7 Hz, 1H), 7.44 (dd, J=9.8, 2.7 Hz, 1H), 7.12 (td, J=8.6, 2.7 Hz, 1H), 6.78 (d, J=8.8 Hz, 1H), 6.73 (d, J=8.8 Hz, 1H), 6.71 (d, J=8.5 Hz, 1H), 5.87 (s, 1H), 5.57 (s, 1H), 5.53 (m, 1H), 4.21 (s, 1H), 3.80 (s, 3H), 2.12 (d, J=1.2 Hz, 3H), 1.36 (s, 6H).

### Example 169

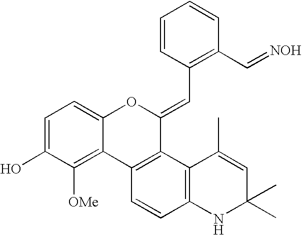

(Z)-5-((E)-2′-(Hydroxyiminomethyl)benzylidene)1,2-dihydro-9-hydroxy-10-methoxy-2,2,4-trimethyl-5H-chromeno[3,4-f]quinoline (Compound 192, Structure 46 of Scheme XII, where R³═H, R⁴═H, R⁵═H, R^{C}═OH).

(Z)-5-(2′-(Piperidinecarbonyl)benzylidene)1,2-dihydro-10-methoxy-2,2,4-trimethyl-9-(triisopropylsilyl)oxy-5H-chromeno[3,4-f]quinoline (Structure 41 of Scheme XI, where R³═H, R⁴═H, R⁵═H).

This compound was prepared according to General Method 3 (EXAMPLE 135) from (Z)-5-(2′-(piperidinecarbonyl)benzylidene)1,2-dihydro-9-hydroxy-10-methoxy-2,2,4-trimethyl-5H-chromeno[3,4-f]quinoline (Compound 158, EXAMPLE 142) to afford (Z)-5-(2′-(piperidinecarbonyl)benzylidene)1,2-dihydro-10-methoxy-2,2,4-trimethyl-9-(triisopropylsilyl)oxy-5H-chromeno[3,4-f]quinoline.

(Z)-5-(2′-Hydroxymethylbenzylidene)1,2-dihydro-10-methoxy-2,2,4-trimethyl-9-(triisopropylsilyl)oxy-5H-chromeno[3,4-f]quinoline (Structure 42 of Scheme XI, where R³═H, R⁴═H, R⁵═H). This compound was prepared according to General Method 6 (EXAMPLE 141) from (Z)-5-(2′-(piperidinecarbonyl)benzylidene)1,2-dihydro-10-methoxy-2,2,4-trimethyl-9-(triisopropylsilyl)oxy-5H-chromeno[3,4-f]quinoline to afford (Z)-5-(2′-hydroxymethylbenzylidene)1,2-dihydro-10-methoxy-2,2,4-trimethyl-9-(triisopropylsilyl)oxy-5H-chromeno[3,4-f]quinoline.

(Z)-5-(2′-Formylbenzylidene)1,2-dihydro-10-methoxy-2,2,4-trimethyl-9-(triisopropylsilyl)oxy-5H-chromeno[3,4-f]quinoline (Structure 44 of Scheme XII, where R³═H, R⁴═H, R⁵═H). This compound was prepared according to General Method 11 (EXAMPLE 167) from (Z)-5-(2′-hydroxymethylbenzylidene)1,2-dihydro-10-methoxy-2,2,4-trimethyl-9-(triisopropylsilyl)oxy-5H-chromeno[3,4-f]quinoline to afford (Z)-5-(2′-formylbenzylidene)1,2-dihydro-10-methoxy-2,2,4-trimethyl-9-(triisopropylsilyl)oxy-5H-chromeno[3,4-f]quinoline.

(Z)-5-((E)-2′-(Hydroxyiminomethyl)benzylidene)1,2-dihydro-9-hydroxy-10-methoxy-2,2,4-trimethyl-5H-chromeno[3,4-f]quinoline (Compound 192, Structure 46 of Scheme XII, where R³═H, R⁴═H, R⁵═H, R^{C}═OH). This compound was prepared by sequential treatment of (Z)-5-(2′-formylbenzylidene)1,2-dihydro-10-methoxy-2,2,4-trimethyl-9-(triisopropylsilyl)oxy-5H-chromeno[3,4-f]quinoline with General Method 12 (EXAMPLE 167) and General Method 10 (EXAMPLE 158) using hydroxylamine hydrochloride to afford Compound 192.

¹H NMR (500 MHz, CDCl₃) δ 8.35 (s, 1H), 8.18 (d, J=8.5 Hz, 1H), 8.06 (dd, J=7.7, 1.0 Hz, 1H), 7.70 (dd, J=7.7, 1.0 Hz, 1H), 7.42 (td, J=7.7, 1.0 Hz, 1H), 7.24 (td, J=7.7, 1.0 Hz, 1H), 7.18 (s, 1H), 6.78 (d, J=8.8 Hz, 1H), 6.75 (d, J=8.8 Hz, 1H), 6.70 (d, J=8.5 Hz, 1H), 5.99 (s, 1H), 5.57 (s, 1H), 5.53 (m, 1H), 4.21 (s, 1H), 3.80 (s, 3H), 2.14 (d, J=1.2 Hz, 3H), 1.36 (s, 6H).

### Example 170

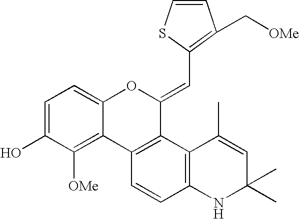

(Z)-5-(2′-(3′-Methoxymethylthienylmethylidene))1,2-dihydro-9-hydroxy-10-methoxy-2,2,4-trimethyl-5H-chromeno[3,4-f]quinoline (Compound 193, Structure 15 of Scheme IV, where X═S, R¹³═H, R^{D}=Me).

General Method 13: Alkylation of an alcohol with an alkyl halide and a base. A solution of the alcohol (1 equiv.), base (10 equiv.) in THF (0.02 to 0.1 M) at 0° C. The reaction suspension was allowed to warm to room temperature, stirred for 0.5 h and re-cooled to 0° C. before the addition of the alkyl halide (10 equiv.). The reaction was allowed to warm to room temperature and stirred for 4 h, a saturated solution of ammonium chloride (50 mL/mmol) added, ethyl acetate (50 mL/mmol) added and the layers separated. The aqueous layer was extracted with ethyl acetate (3×25 mL/mmol), the combined organic extracts washed with a saturated solution of ammonium chloride (200 mL/mmol), dried (Na₂SO₄) and concentrated under reduced pressure. Purification by flash chromatography, eluting with ethyl acetate:hexanes, afforded the desired alcohol as a yellow oil. In certain instances, less base can be required.

(Z)-5-(2′-(3′-Methoxymethylthienylmethylidene))1,2-dihydro-10-methoxy-2,2,4-trimethyl-9-(triisopropylsilyl)oxy-5H-chromeno[3,4-f]quinoline (Structure 14 of Scheme IV, where X═S, R¹³═H, R^{D}=Me). This compound was prepared according to General Method 13 (Example 170) from (Z)-5-(2′-(3′-hydroxymethylthienylmethylidene))-1,2-dihydro-10-methoxy-2,2,4-trimethyl-9-(triisopropylsilyl)oxy-5H-chromeno[3,4-f]quinoline (EXAMPLE 167), 60% sodium hydride in mineral oil, and iodomethane in THF to afford (Z)-5-(2′-(3′-methoxymethylthienylmethylidene))1,2-dihydro-10-methoxy-2,2,4-trimethyl-9-(triisopropylsilyl)oxy-5H-chromeno[3,4-f]quinoline.

(Z)-5-(2′-(3′-Methoxymethylthienylmethylidene))1,2-dihydro-9-hydroxy-10-methoxy-2,2,4-trimethyl-5H-chromeno[3,4-f]quinoline (Compound 193, Structure 15 of Scheme IV, where X═S, R¹³═H, R^{D}=Me). This compound was prepared according to General Method 12 (EXAMPLE 167) from (Z)-5-(2′-(3′-methoxymethylthienylmethylidene))1,2-dihydro-10-methoxy-2,2,4-trimethyl-9-(triisopropylsilyl)oxy-5H-chromeno[3,4-f]quinoline to afford Compound 193. ¹H NMR (500 MHz, Acetone-d₆) δ 8.32 (d, J=8.6 Hz, 1H), 7.78 (s, 1H), 7.33 (dd, J=5.3, 0.5 Hz, 1H), 7.00 (d, J=5.3 Hz, 1H), 7.00 (d, J=8.6 Hz, 1H), 6.81 (d, J=8.6 Hz, 1H), 6.80 (d, J=8.6 Hz, 1H), 6.22 (d, J=0.5 Hz, 1H), 5.88 (s, 1H), 5.55 (q, J=1.3 Hz, 1H), 4.42 (s, 2H), 3.77 (s, 3H), 3.30 (s, 3H), 2.07 (d, J=1.3 Hz, 3H), 1.34 (s, 6H).

### Example 171

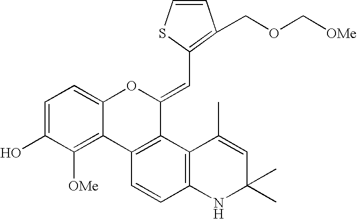

(Z)-5-(2′-(3′-(Methoxymethoxymethyl)thienylmethylidene))1,2-dihydro-9-hydroxy-10-methoxy-2,2,4-trimethyl-5H-chromeno[3,4-f]quinoline

Compound 194, Structure 15 of Scheme IV, where X═S, R¹³═H, R^{D}=methoxymethyl).

(Z)-5-(2′-(3′-(Methoxymethoxymethyl)thienylmethylidene))1,2-dihydro-10-methoxy-2,2,4-trimethyl-9-(triisopropylsilyl)oxy-5H-chromeno[3,4-f]quinoline (Structure 14 of Scheme IV, where X═S, R¹³═H, R^{D}=methoxymethyl).

This compound was prepared according to General Method 13 (Example 170) from (Z)-5-(2′-(3′-(hydroxymethyl)thienylmethylidene))-1,2-dihydro-10-methoxy-2,2,4-trimethyl-9-(triisopropylsilyl)oxy-5H-chromeno[3,4-f]quinoline (EXAMPLE 167), diisopropylethylamine and MOM-Cl in dichloromethane to afford (Z)-5-(2′-(3′-methoxymethoxymethylthienylmethylidene))1,2-dihydro-10-methoxy-2,2,4-trimethyl-9-(triisopropylsilyl)oxy-5H-chromeno[3,4-f]quinoline.

(Z)-5-(2′-(3′-(Methoxymethoxymethyl)thienylmethylidene))1,2-dihydro-9-hydroxy-10-methoxy-2,2,4-trimethyl-5H-chromeno[3,4-f]quinoline (Compound 194, Structure 15 of Scheme IV, where X═S, R13=H, RD=methoxymethyl). This compound was prepared according to General Method 12 (EXAMPLE 167) from (Z)-5-(2′-(3′-(methoxymethoxymethyl)thienylmethylidene))1,2-dihydro-10-methoxy-2,2,4-trimethyl-9-(triisopropylsilyl)oxy-5H-chromeno[3,4-f]quinoline to afford Compound 194. ¹H NMR (500 MHz, Acetone) δ 8.31 (d, J=8.6 Hz, 1H), 7.78 (s, 1H), 7.35 (dd, J=5.2, 0.6 Hz, 1H), 7.04 (d, J=5.2 Hz, 1H), 7.00 (d, J=8.6 Hz, 1H), 6.81 (d, J=8.6 Hz, 1H), 6.80 (d, J=8.6 Hz, 1H), 6.21 (d, J=0.6 Hz, 1H), 5.90 (s, 1H), 5.54 (q, J=1.3 Hz, 1H), 4.62 (s, 2H), 4.56 (s, 2H), 3.77 (s, 3H), 3.32 (s, 3H), 2.07 (d, J=1.3 Hz, 3H), 1.34 (s, 6H).

### Example 172

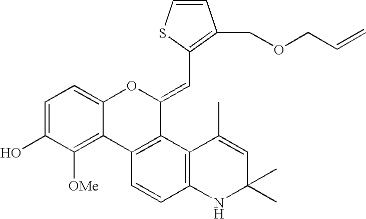

(Z)-5-(2′-(3′-(Prop-2″-enyloxymethyl)thienylmethylidene))1,2-dihydro-9-hydroxy-10-methoxy-2,2,4-trimethyl-5H-chromeno[3,4-f]quinoline (Compound 195, Structure 15 of Scheme IV, where X═S, R¹³═H, R^{D}=allyl).

(Z)-5-(2-(3′-(Prop-2″-enyloxymethyl)thienylmethylidene))1,2-dihydro-10-methoxy-2,2,4-trimethyl-9-(triisopropylsilyl)oxy-5H-chromeno[3,4-f]quinoline (Structure 14 of Scheme IV, where X═S, R¹³═H, R^{D}=allyl).

This compound was prepared according to General Method 13 (Example 170) from (Z)-5-(2′-(3″-(hydroxymethyl)thienylmethylidene))-1,2-dihydro-10-methoxy-2,2,4-trimethyl-9-(triisopropylsilyl)oxy-5H-chromeno[3,4-f]quinoline (EXAMPLE 167), 60% sodium hydride in mineral oil, and allyl bromide in THF to afford (Z)-5-(2′-(3′-(prop-2″-enyloxymethyl)thienylmethylidene))1,2-dihydro-10-methoxy-2,2,4-trimethyl-9-(triisopropylsilyl)oxy-5H-chromeno[3,4-f]quinoline.

(Z)-5-(2′-(3′-(Prop-2″-enyloxymethyl)thienylmethylidene))1,2-dihydro-9-hydroxy-10-methoxy-2,2,4-trimethyl-5H-chromeno[3,4-f]quinoline (Compound 195, Structure 15 of Scheme IV, where X═S, R¹³═H, R^{D}=allyl). This compound was prepared according to General Method 12 (EXAMPLE 167) from (Z)-5-(2′-(3′-(prop-2″-enyloxymethyl)thienylmethylidene))1,2-dihydro-10-methoxy-2,2,4-trimethyl-9-(triisopropylsilyl)oxy-5H-chromeno[3,4-f]quinoline to afford Compound 195. ¹H NMR (500 MHz, Acetone-d₆) δ 8.31 (d, J=8.8 Hz, 1H), 7.77 (s, 1H), 7.34 (dd, J=5.2, 0.6 Hz, 1H), 7.02 (d, J=5.2 Hz, 1H), 7.00 (d, J=8.8 Hz, 1H), 6.81 (d, J=8.8 Hz, 1H), 6.80 (d, J=8.8 Hz, 1H), 6.21 (d, J=0.6 Hz, 1H), 5.94 (ddt, J=17.2, 10.4, 5.3 Hz, 1H), 5.90 (br s, 1H), 5.54 (q, J=1.5 Hz, 1H), 5.27 (ddt, J=17.2, 1.9, 1.7 Hz, 1H), 5.14 (ddt, J=10.4, 1.9, 1.5 Hz, 1H), 4.49 (s, 2H), 4.01 (ddd, J=5.3, 1.7, 1.5 Hz, 2H), 3.77 (s, 3H), 2.07 (d, J=1.5 Hz, 3H), 1.34 (s, 6H).

### Example 173

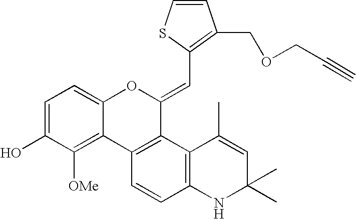

(Z)-5-(2′-(3′-(Prop-2″-ynloxymethyl)thienylmethylidene))1,2-dihydro-9-hydroxy-10-methoxy-2,2,4-trimethyl-5H-chromeno[3,4-f]quinoline (Compound 196, Structure 15 of Scheme IV, where X═S, R¹³═H, R^{D}=prop-2-ynl).

(Z)-5-(2′-(3′-(Prop-2″-ynloxymethyl)thienylmethylidene))1,2-dihydro-10-methoxy-2,2,4-trimethyl-9-(triisopropylsilyl)oxy-5H-chromeno[3,4-f]quinoline (Structure 14 of Scheme IV, where X═S, R¹³═H, R^{D}=prop-2-ynl).

This compound was prepared according to General Method 13 (EXAMPLE 170) from (Z)-5-(2′-(3′-(hydroxymethyl)thienylmethylidene))-1,2-dihydro-10-methoxy-2,2,4-trimethyl-9-(triisopropylsilyl)oxy-5H-chromeno[3,4-f]quinoline (EXAMPLE 167), 60% sodium hydride in mineral oil and propargyl bromide in THF to afford (Z)-5-(2′-(3′-(prop-2″-ynloxymethyl)thienylmethylidene))1,2-dihydro-10-methoxy-2,2,4-trimethyl-9-(triisopropylsilyl)oxy-5H-chromeno[3,4-f]quinoline.

(Z)-5-(2′-(3′-(Prop-2″-ynloxymethyl)thienylmethylidene))1,2-dihydro-9-hydroxy-10-methoxy-2,2,4-trimethyl-5H-chromeno[3,4-f]quinoline (Compound 196, Structure 15 of Scheme IV, where X═S, R¹³═H, R^{D}=prop-2-ynl). This compound was prepared according to General Method 12 (EXAMPLE 167) from (Z)-5-(2′-(3′-(prop-2″-ynloxymethyl)thienylmethylidene))1,2-dihydro-10-methoxy-2,2,4-trimethyl-9-(triisopropylsilyl)oxy-5H-chromeno[3,4-f]quinoline to afford Compound 196. ¹H NMR (500 MHz, Acetone-d₆) δ 8.31 (d, J=8.7 Hz, 1H), 7.77 (s, 1H), 7.35 (dd, J=5.2, 0.6 Hz, 1H), 7.03 (d, J=5.2 Hz, 1H), 7.00 (d, J=8.7 Hz, 1H), 6.81 (d, J=8.7 Hz, 1H), 6.80 (d, J=8.7 Hz, 1H), 6.21 (d, J=0.6 Hz, 1H), 5.89 (s, 1H), 5.58 (q, J=1.3 Hz, 1H), 4.59 (s, 2H), 4.18 (d, J=2.4 Hz, 2H), 3.77 (s, 3H), 2.99 (t, J=2.4 Hz, 1H), 2.07 (d, J=1.3 Hz, 3H), 1.34 (s, 6H).

### Example 174

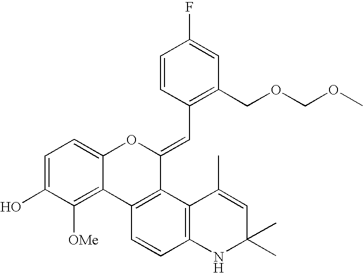

(Z)-5-(4′-Fluoro-2′-(methoxymethoxymethyl)benzylidene)1,2-dihydro-9-hydroxy-10-methoxy-2,2,4-trimethyl-5H-chromeno[3,4-f]quinoline (Compound 197, Structure 43 of Scheme XI, where R³═H, R⁴═F, R⁵═H, and R^{D}32 methoxymethyl).

This compound was prepared according to General Method 13 (EXAMPLE 170) from (Z)-5-(4′-fluoro-2′-hydroxymethylbenzylidene)1,2-dihydro-10-methoxy-2,2,4-trimethyl-9-(triisopropylsilyl)oxy-5H-chromeno[3,4-f]quinoline (EXAMPLE 168), diisopropylethylamine, and MOM-Cl in dichloromethane, followed by treatment according to General Method 12 (EXAMPLE 167) to afford Compound 197. ¹H NMR (500 MHz, CDCl₃) δ 8.19 (dd, J=8.6, 5.9 Hz, 1H), 8.16 (d, J=8.5 Hz, 1H), 7.12 (dd, J=9.6, 2.9 Hz, 1H), 7.05 (td, J=8.6, 2.9 Hz, 1H), 6.80 (d, J=8.7 Hz, 1H), 6.78 (d, J=8.7 Hz, 1H), 5.81 (s, 1H), 5.58 (br, 1H), 5.49 (d, J=1.2 Hz, 1H), 4.66 (s, 2H), 4.53 (s, 2H), 4.53 (s, 2H), 4.21 (s, 1H), 3.34 (s, 3H), 2.12 (d, J=1.2 Hz, 3H), 1.35 (s, 6H).

### Example 175

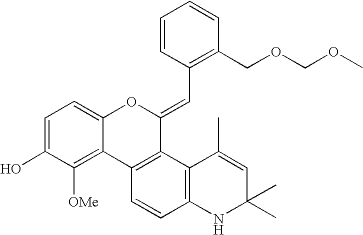

(Z)-5-(2′-(Methoxymethoxymethyl)benzylidene)1,2-dihydro-9-hydroxy-10-methoxy-2,2,4-trimethyl-5H-chromeno[3,4-f]quinoline (Compound 198, Structure 43 of Scheme XI, where R³═H, R⁴═H, R⁵═H, and R^{D}=methoxymethyl).

This compound was prepared according to General Method 13 (EXAMPLE 170) from (Z)-5-(2′-hydroxymethylbenzylidene)1,2-dihydro-10-methoxy-2,2,4-trimethyl-9-(triisopropylsilyl)oxy-5H-chromeno[3,4-f]quinoline (EXAMPLE 169) followed by treatment according to General Method 12 (EXAMPLE 167) to afford Compound 198. ¹H NMR (500 MHz, CDCl₃) δ 8.26 (dd, J=7.6, 1.0 Hz, 1H), 8.16 (d, J=8.5 Hz, 1H), 7.39-7.34 (m, 2H), 7.22 (td, J=7.6, 1.0 Hz, 1H), 6.82 (d, J=8.7 Hz, 1H), 6.79 (d, J=8.7 Hz, 1H), 6.68 (d, J=8.5 Hz, 1H), 5.96 (s, 1H), 5.49 (q, J=1.2 Hz, 1H), 4.65 (s, 2H), 4.56 (s, 2H), 4.20 (s, 1H), 3.79 (s, 3H), 3.33 (s, 3H), 2.14 (d, J=1.2 Hz, 3H), 1.35 (s, 6H).

### Example 176

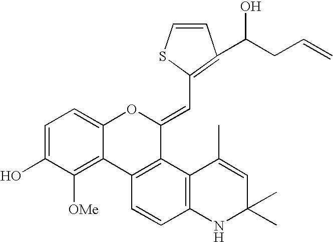

(±)-(Z)-5-(2′-(3′-(1″-Hydroxybut-3″-enyl)thienylmethylidene))1,2-dihydro-9-hydroxy-10-methoxy-2,2,4-trimethyl-5H-chromeno[3,4-f]quinoline (Compound 199, Structure 20 of Scheme V, where X═S, R¹³═H, R^{A}=allyl).

This compound was prepared according to General Method 8A (EXAMPLE 152) from (Z)-5-(2′-(3″-formylthienylmethylidene))-1,2-dihydro-10-methoxy-2,2,4-trimethyl-9-(triisopropylsilyl)oxy-5H-chromeno[3,4-f]quinoline (EXAMPLE 167) and allyl magnesium bromide followed by treatment according to General Method 12 (EXAMPLE 167) to afford Compound 199. ¹H NMR (500 MHz, Acetone-d₆) δ 8.30 (d, J=8.8 Hz, 1H), 7.77 (s, 1H), 7.32 (d, J=5.3 Hz, 1H), 7.12 (d, J=5.3 Hz, 1H), 6.99 (d, J=8.8 Hz, 1H), 6.80 (d, J=8.8 Hz, 1H), 6.79 (d, J=8.8 Hz, 1H), 6.17 (s, 1H), 5.89 (s, 1H), 5.81 (ddt, J=17.2, 10.0, 7.0 Hz, 1H), 5.53 (q, J=1.2 Hz, 1H), 5.06 (dm, J=17.2 Hz, 1H), 4.98 (dm, J=10.0 Hz, 1H), 4.86 (m, 1H), 4.23 (d, J=4.1 Hz, 1H), 3.77 (s, 3H), 2.52 (m, 1H), 2.40 (m, 1H), 2.06 (d, J=1.2 Hz, 3H), 1.33 (s, 6H).

### Example 177

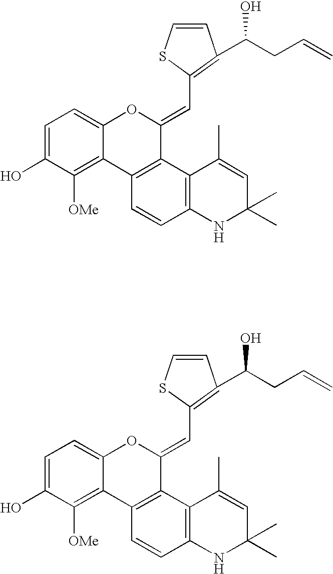

(+)-(Z)-5-(2′-(3′-(1″-Hydroxybut-3″-enyl)thienylmethylidene))1,2-dihydro-9-hydroxy-10-methoxy-2,2,4-trimethyl-5H-chromeno[3,4-f]quinoline (Compound 200, (+)-Structure 20 of Scheme V, where X═S, R¹³═H, R^{A}=allyl), and (−)-(Z)-5-(2′-(3′-(1″-Hydroxybut-3″-enyl)thienylmethylidene))1,2-dihydro-9-hydroxy-10-methoxy-2,2,4-trimethyl-5H-chromeno[3,4-f]quinoline (Compound 201, (−)-Structure 20 of Scheme V, where X═S, R¹³═H, R^{A}=allyl).

General Method 14. Separation of a racemic alcohol into its individual enantiomers. The alcohol can be separated using a semi-prep Chiracel OD column (10×250 mm) eluted with 92:8 hexanes:ethanol at an elution rate of 3.5 mL/min to afford the desired (+)- and (−)-enantiomers. These compounds were prepared according to General Method 14 and afforded Compound 200 (first peak) and Compound 201 (2^{nd} peak).

### Example 178

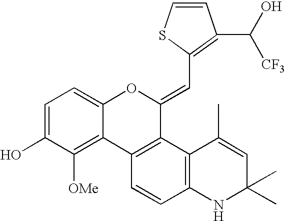

(±)-(Z)-5-(2′-(3′-(1″-Hydroxy-2″,2″,2″-trifluoroethyl)thienylmethylidene))1,2-dihydro-9-hydroxy-10-methoxy-2,2,4-trimethyl-5H-chromeno[3,4-f]quinoline (Compound 202, Structure 20 of Scheme V, where X═S, R¹³═H, R^{A}=trifluoromethyl).

General Method 15. Addition of a trifluoromethyl group generated from (trifluoromethyl)trimethylsilane and a fluoride source. Trifluoromethyltrimethylsilane (10 equiv) was added to a solution of the carbonyl compound (1 equiv) in THF (0.01-0.1 M). The solution was cooled to 0° C. before the dropwise addition of 1M tetrabutylammonium fluoride in THF (5 equiv) over 0.2 h. The reaction solution was stirred for an additional 0.2 h, a saturated solution of ammonium chloride (75 mL/mmol) added, ethyl acetate (75 mL/mmol) added and the layers separated. The aqueous layer was extracted with ethyl acetate (3×40 mL/mmol), the combined organic extracts washed with a saturated solution of ammonium chloride (100 mL/mmol), dried (Na₂SO₄) and concentrated under reduced pressure. Purification by flash chromatography, eluting with ethyl acetate:hexanes afforded the desired alcohol as a yellow oil.

(Z)-5-(2′-(3′-Piperidinecarbonylthienylmethylidene))1,2-dihydro-10-methoxy-9-methoxymethoxy-2,2,4-trimethyl-5H-chromeno[3,4-f]quinoline (Structure 12 of Scheme IV, where X═S, PG=methoxymethoxy, R¹³═H, R¹⁴R¹⁵=—(CH₂)5-). Methoxymethyl ether (4.0 mL, 53 mmol) was added to a solution of Compound 140 (EXAMPLE 124) (4.0 g, 7.6 mmol) in dichloromethane (200 mL). Tetrabutylammonium hydroxide (1.0 ml) and a 6 M sodium hydroxide solution (1.0 mL) were added and the reaction solution stirred at room temperature for 1 h. The reaction was diluted with water (200 mL), the layers separated and the aqueous layer extracted with ethyl acetate (3×50 mL). The combined organic extracts were washed with 1 M hydrochloric acid (400 mL), a saturated solution of ammonium chloride (400 mL), dried (Na₂SO₄) and concentrated under reduced pressure. Purification by flash chromatography, eluting with ethyl acetate:hexanes afforded (Z)-5-(2′-(3′-piperidinecarbonylthienylmethylidene))1,2-dihydro-10-methoxy-9-methoxymethoxy-2,2,4-trimethyl-5H-chromeno[3,4-f]quinoline (3.6 g, 83%) as a yellow oil.

(Z)-5-(2′-(3′-(Hydroxymethyl)thienylmethylidene))-1,2-dihydro-10-methoxy-9-methoxymethoxy-2,2,4-trimethyl-5H-chromeno[3,4-f]quinoline (Structure 13 of Scheme IV, where X═S, PG=methoxymethoxy, R³═H). This compound was prepared by General Method 6 (EXAMPLE 141) from (Z)-5-(2′-(3′-(piperidinecarbonyl)thienylmethylidene))-1,2-dihydro-10-methoxy-9-methoxymethoxy-2,2,4-trimethyl-5H-chromeno[3,4-f]quinoline to afford (Z)-5-(2′-(3′-hydroxymethylthienylmethylidene))-1,2-dihydro-10-methoxy-9-methoxymethoxy-2,2,4-trimethyl-5H-chromeno[3,4-f]quinoline.

(Z)-5-(2′-(3″-Formylthienylmethylidene))-1,2-dihydro-10-methoxy-9-methoxymethoxy-2,2,4-trimethyl-5H-chromeno[3,4-f]quinoline (Structure 18 of Scheme V, where X═S, PG=methoxymethoxy, R¹³═H). This compound was prepared according to General Method 11 (EXAMPLE 167) from (Z)-5-(2′-(3′-hydroxymethylthienylmethylidene))-1,2-dihydro-10-methoxy-9-methoxymethoxy-2,2,4-trimethyl-5H-chromeno[3,4-f]quinoline to afford (Z)-5-(2′-(3″-formylthienylmethylidene))-1,2-dihydro-10-methoxy-9-methoxymethoxy-2,2,4-trimethyl-5H-chromeno[3,4-f]quinoline.

(±)-(Z)-5-(2′-(3′-(1″-Hydroxy-2″,2″,2″-trifluoroethyl)thienylmethylidene))-1,2-dihydro-10-methoxy-9-methoxymethoxy-2,2,4-trimethyl-5H-chromeno[3,4-f]quinoline (Structure 19 of Scheme V, where X═S, PG=methoxymethoxy, R¹³═H, R^{A}=trifluoromethyl This compound was prepared according to General Method 15 (EXAMPLE 178) from (Z)-5-(2′-(3′-formylthienylmethylidene))-1,2-dihydro-10-methoxy-9-methoxymethoxy-2,2,4-trimethyl-5H-chromeno[3,4-f]quinoline (EXAMPLE 167) and (trifluoromethyl)trimethylsilane to afford (±)-(Z)-5-(2′-(3′-(1″-Hydroxy-2″,2″,2″-trifluoroethyl)thienylmethylidene))1,2-dihydro-10-methoxy-9-methoxymethoxy-2,2,4-trimethyl-5H-chromeno[3,4-f]quinoline.

(±)-(Z)-5-(2′-(3′-(1″-Hydroxy-2″,2″,2″-trifluoroethyl)thienylmethylidene))-1,2-dihydro-9-hydroxy-10-methoxy-2,2,4-trimethyl-5H-chromeno[3,4-f]quinoline (Compound 202, Structure 20 of Scheme V, where X═S, R¹³═H, R^{A}-trifluoromethyl). A solution of (±)-(Z)-5-(2′-(3′-(1″-Hydroxy-2″,2″,2″-trifluoroethyl)thienylmethylidene))1,2-dihydro-10-methoxy-9-methoxymethoxy-2,2,4-trimethyl-5H-chromeno[3,4-f]quinoline in 10% HCl:methanol (10 mg starting material/1 mL solution) was stirred at rt for 3 h. The reaction was diluted with water, extracted with ethyl acetate, and the combined organic layer was washed sequentially with saturated sodium bicarbonate and saturated ammonium chloride, dried over sodium sulfate, and concentrated under reduced pressure. Flash chromatography (ethyl acetate:hexanes) afforded Compound 202. ¹H NMR (500 MHz, Acetone-d₆) δ 8.32 (d, J=8.8 Hz, 1H), 7.82 (s, 1H), 7.43 (d, J=5.5 Hz, 1H), 7.21 (d, J=5.5 Hz, 1H), 7.01 (d, J=8.8 Hz, 1H), 6.82 (d, J=8.8 Hz, 2H), 6.22 (s, 1H), 5.93 (s, 1H), 5.79 (d, J=5.7 Hz, 1H), 5.52 (q, J=1.6 Hz, 1H), 5.30 (dq, J=5.7, 7.2 Hz, 1H), 3.77 (s, 3H), 3.77 (d, J=1.6 Hz, 3H), 1.32 (s, 6H).

### Example 179

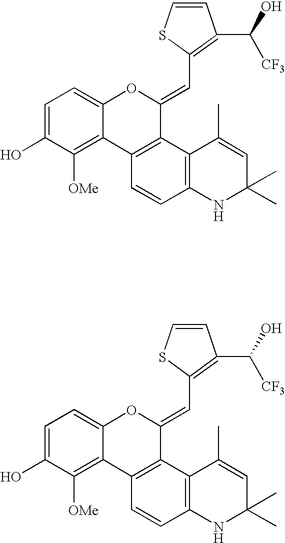

(+)-(Z)-5-(2′-(3′-(1″-Hydroxy-2″,2″,2″-trifluoroethyl)-thienylmethylidene))1,2-dihydro-9-hydroxy-10-methoxy-2,2,4-trimethyl-5H-chromeno[3,4-f]quinoline (Compound 203, (+)-Structure 20 of Scheme V, where X═S, R¹³═H, R^{A}=trifluoromethyl), and (−)-(Z)-5-(2′-(3′-(1″-Hydroxy-2″,2″,2″-trifluoroethyl)thienylmethylidene))1,2-dihydro-9-hydroxy-10-methoxy-2,2,4-trimethyl-5H-chromeno[3,4-f]quinoline (Compound 204, (−)-Structure 20 of Scheme V, where X═S, R¹³═H, R^{A}=trifluoromethyl).

These compounds were prepared according to General Method 14 (EXAMPLE 177) from Compound 202 to afford Compound 203 (t_{R}=41 min) and Compound 204 (t_{R}=54 min).

### Example 180

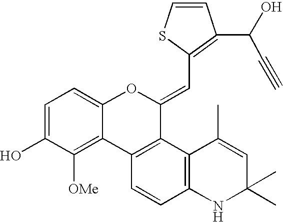

(±)-(Z)-5-(2′-(3′-(1″-Hydroxyprop-2″-ynyl)thienylmethylidene))1,2-dihydro-9-hydroxy-10-methoxy-2,2,4-trimethyl-5H-chromeno[3,4-f]quinoline (Compound 205, Structure 20 of Scheme V, where X═S, R¹³═H, R^{A}=ethynyl).

This compound was prepared according to General Method 8A (EXAMPLE 152) from (Z)-5-(2′-(3″-formylthienylmethylidene))-1,2-dihydro-10-methoxy-2,2,4-trimethyl-9-(triisopropylsilyl)oxy-5H-chromeno[3,4-f]quinoline (EXAMPLE 167) and lithium trimethylsilylacetylide, followed by treatment according to General Method 12 (EXAMPLE 167) to afford Compound 205. ¹H NMR (500 MHz, Acetone-d₆) δ 8.30 (d, J=8.6 Hz, 1H), 7.80 (s, 1H), 7.33 (dd, J=5.2, 0.5 Hz, 1H), 7.21 (d, J=5.2 Hz, 1H), 7.00 (d, J=8.8 Hz, 1H), 6.80 (d, J=8.8 Hz, 1H), 6.80 (d, J=8.6 Hz, 1H), 6.40 (d, J=0.5 Hz, 1H), 5.89 (s, 1H), 5.58 (dd, J=5.5, 2.3 Hz, 1H), 5.53 (q, J=1.2 Hz, 1H), 4.94 (d, J=5.5 Hz, 1H), 3.77 (s, 3H), 3.03 (d, J=2.3 Hz, 1H), 2.08 (d, J=1.2 Hz, 3H), 1.33 (s, 6H).

### Example 181

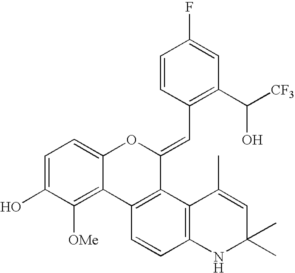

(±)-(Z)-5-(4′-fluoro-2′-(2″,2″,2″-Trifluoro-1″-hydroxyethyl)benzylidene)1,2-dihydro-9-hydroxy-10-methoxy-2,2,4-trimethyl-5H-chromeno[3,4-f]quinoline (Compound 206, Structure 45 of Scheme XI, where R³═H, R⁴═F, R⁵═H).

This compound was prepared according to General Method 15 (EXAMPLE 178) from (Z)-5-(4′-fluoro-2′-formylbenzylidene)1,2-dihydro-10-methoxy-2,2,4-trimethyl-9-(triisopropylsilyl)oxy-5H-chromeno[3,4-f]quinoline (EXAMPLE 168) followed by treatment with General Method 12 (EXAMPLE 167) to afford Compound 206. ¹H NMR (500 MHz, CDCl₃) δ 8.20 (d, J=8.6 Hz, 1H), 8.06 (m, 1H), 7.39 (dd, J=9.9, 2.8 Hz, 1H), 7.13 (td, J=8.4, 2.8 Hz, 1H), 6.78 (d, J=8.7 Hz, 1H), 6.72 (d, J=8.7 Hz, 1H), 6.71 (d, J=8.6 Hz, 1H), 5.78 (s, 1H), 5.73 (s, 1H), 5.51 (q, J=1.2 Hz, 1H), 5.31 (m, 1H), 5.28 (m, 1H), 4.29 (s, 1H), 3.80 (s, 3H), 2.10 (m, 3H), 1.36 (s, 6H).

### Example 182

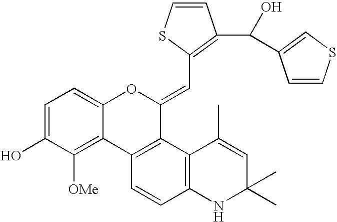

(±)-(Z)-5-(2′-(3′-(Hydroxythien-3″-ylmethyl)thienylmethylidene))1,2-dihydro-9-hydroxy-10-methoxy-2,2,4-trimethyl-5H-chromeno[3,4-f]quinoline (Compound 207, Structure 20 of Scheme V, where X═S, R¹³═H, R^{A}=thien-3-yl).

This compound was prepared according to General Method 8A (EXAMPLE 152) from (Z)-5-(2′-(3′-formylthienylmethylidene))-1,2-dihydro-10-methoxy-2,2,4-trimethyl-9-(triisopropylsilyl)oxy-5H-chromeno[3,4-f]quinoline (EXAMPLE 167) and 3-thienylmagnesium iodide, followed by treatment according to General Method 12 (EXAMPLE 167) to afford Compound 207. ¹H NMR (500 MHz, CDCl₃) δ 8.17 (d, J=8.6 Hz, 1H), 7.27 (m, 1H), 7.24 (d, J=5.1 Hz, 1H), 7.19 (s, 1H), 7.07 (d, J=5.1 Hz, 1H), 7.04 (d, J=8.8 Hz, 1H), 6.97 (m, 1H), 6.84 (d, J=8.8 Hz, 1H), 6.66 (d, J=8.6 Hz, 1H), 6.12 (s, 1H), 6.04 (s, 1H), 5.57 (s, 1H), 5.49 (s, 1H), 4.20 (s, 1H), 3.77 (s, 3H), 1.91 (s, 3H), 1.35 (s, 6H).

### Example 183

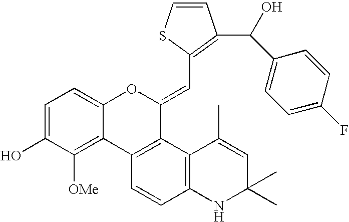

(±)-(Z)-5-(2′-(3′-((4″-Fluorophenyl)hydroxymethyl)thienylmethylidene))1,2-dihydro-9-hydroxy-10-methoxy-2,2,4-trimethyl-5H-chromeno[3,4-f]quinoline (Compound 208, Structure 20 of Scheme V, where X═S, R¹³═H, R^{A}=4-fluorophenyl).

This compound was prepared according to General Method 8A (EXAMPLE 152) from (Z)-5-(2′-(3″-formylthienylmethylidene))-1,2-dihydro-10-methoxy-2,2,4-trimethyl-9-(triisopropylsilyl)oxy-5H-chromeno[3,4-f]quinoline (EXAMPLE 167) and 4-fluoromagnesium bromide, followed by treatment according to General Method 12 (EXAMPLE 167) to afford Compound 208. ¹H NMR (300 MHz, CDCl₃) δ 8.17 (d, J=8.6 Hz, 1H), 7.32 (dd, J=8.5, 5.5 Hz, 2H), 7.24 (d, J=5.3 Hz, 1H), 7.06 (d, J=5.3 Hz, 1H), 7.04 (d, J=8.8 Hz, 1H), 6.99 (t, J=8.5 Hz, 2H), 6.84 (d, J=8.8 Hz, 1H), 6.66 (d, J=8.6 Hz, 1H), 6.06 (s, 1H), 5.97 (s, 1H), 5.57 (s, 1H), 5.45 (s, 1H), 4.22 (s, 1H), 3.76 (s, 3H), 2.05 (s, 1H), 1.88 (m, 3H), 1.35 (s, 6H).

### Example 184

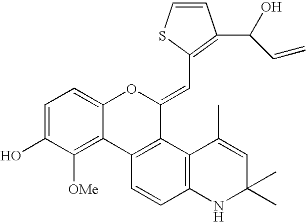

(±)-(Z)-5-(2′-(3′-(1″-Hydroxyallyl)thienylmethylidene))1,2-dihydro-9-hydroxy-10-methoxy-2,2,4-trimethyl-5H-chromeno[3,4-f]quinoline (Compound 209, Structure 20 of Scheme V, where X═S, R¹³═H, R^{A}=1-hydroxyallyl).

This compound was prepared according to General Method 8A (EXAMPLE 152) from (Z)-5-(2′-(3′-formylthienylmethylidene))-1,2-dihydro-10-methoxy-2,2,4-trimethyl-9-(triisopropylsilyl)oxy-5H-chromeno[3,4-f]quinoline (EXAMPLE 167) and vinyl magnesium bromide, followed by treatment according to General Method 12 (EXAMPLE 167) to afford Compound 209. ¹H NMR (300 MHz, CD₃OD) δ 8.31 (d, J=8.6 Hz, 1H), 7.27 (d, J=5.2 Hz, 1H), 7.05 (d, J=5.2 Hz, 1H), 6.94 (d, J=8.6 Hz, 1H), 6.74 (d, J=8.6 Hz, 1H), 6.73 (d, J=8.6 Hz, 1H), 6.17 (s, 1H), 6.00 (ddd, J=16.7, 10.2, 6.3 Hz, 1H), 5.50 (m, 1H), 5.29-5.20 (m, 2H), 5.09 (d, J=10.2 Hz, 1H), 3.76 (s, 3H), 2.04 (m, 3H), 1.31 (s, 6H).

### Example 185

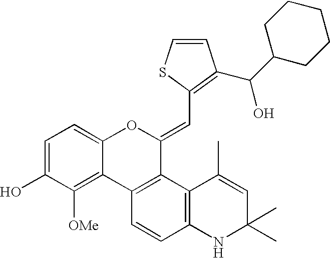

(±)-(Z)-5-(2′-(3′-(Cyclohexylhydroxymethyl)thienylmethylidene))1,2-dihydro-9-hydroxy-10-methoxy-2,2,4-trimethyl-5H-chromeno[3,4-f]quinoline (Compound 210, Structure 20 of Scheme V, where X═S, R¹³═H, R^{A}=cyclohexyl).

This compound was prepared according to General Method 8A (EXAMPLE 152) from (Z)-5-(2′-(3″-formylthienylmethylidene))-1,2-dihydro-10-methoxy-2,2,4-trimethyl-9-(triisopropylsilyl)oxy-5H-chromeno[3,4-f]quinoline (EXAMPLE 167) and cyclohexylmagnesium bromide, followed by treatment according to General Method 12 (EXAMPLE 167) to afford Compound 210. ¹H NMR (300 MHz, CD₃OD) δ 8.31 (d, J=8.7 Hz, 1H), 7.27 (d, J=5.2 Hz, 1H), 7.02 (d, J=5.2 Hz, 1H), 6.92 (d, J=8.7 Hz, 1H), 6.74 (d, J=8.7 Hz, 1H), 6.73 (d, J=8.7 Hz, 1H), 6.07 (s, 1H), 5.50 (m, 1H), 4.47 (d, J=7.5 Hz, 1H), 3.76 (s, 3H), 2.05 (m, 3H), 1.81-1.53 (m, 5H), 1.31 (s, 6H), 1.25-0.86 (m, 6H).

### Example 186

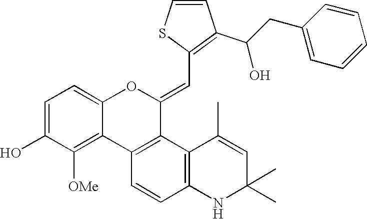

(±)-(Z)-5-(2′-(3′-(1″-Hydroxy-2″-phenylethyl)thienylmethylidene))1,2-dihydro-9-hydroxy-10-methoxy-2,2,4-trimethyl-5H-chromeno[3,4-f]quinoline (Compound 211, Structure 20 of Scheme V, where X═S, R¹³═H, R^{A}=benzyl).

This compound was prepared according to General Method 8A (EXAMPLE 152) from (Z)-5-(2′-(3′-formylthienylmethylidene))-1,2-dihydro-10-methoxy-2,2,4-trimethyl-9-(triisopropylsilyl)oxy-5H-chromeno[3,4-f]quinoline (EXAMPLE 167) and benzyl magnesium chloride, followed by treatment according to General Method 12 (EXAMPLE 167) to afford Compound 211. ¹H NMR (500 MHz, CDCl₃) δ 8.19 (d, J=8.6 Hz, 1H), 7.30-7.27 (m, 2H), 7.27 (d, J=5.3 Hz, 1H), 7.25-7.20 (m, 3H), 7.15 (d, J=5.3 Hz, 1H), 7.05 (d, J=8.7 Hz, 1H), 6.85 (d, J=8.7 Hz, 1H), 6.69 (d, J=8.6 Hz, 1H), 6.12 (s, 1H), 5.57 (m, 1H), 5.55 (q, J=1.2 Hz, 1H), 5.07 (m, 1H), 4.22 (m, 1H), 3.78 (s, 3H), 2.99 (m, 2H), 2.06 (m, 3H), 1.25 (s, 6H).

### Example 187

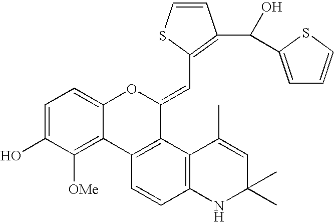

(±)-(Z)-5-(2′-(3′-(Hydroxythien-2″-ylmethyl)thienylmethylidene))1,2-dihydro-9-hydroxy-10-methoxy-2,2,4-trimethyl-5H-chromeno[3,4-f]quinoline (Compound 212, Structure 20 of Scheme V, where X═S, R¹³═H, R^{A}=2-thienyl).

This compound was prepared according to General Method 8A (EXAMPLE 152) from (Z)-5-(2′-(3′-formylthienylmethylidene))-1,2-dihydro-10-methoxy-2,2,4-trimethyl-9-(triisopropylsilyl)oxy-5H-chromeno[3,4-f]quinoline (EXAMPLE 167) and 2-thienylmagnesium bromide, followed by treatment according to General Method 12 (EXAMPLE 167) to afford Compound 212. ¹H NMR (500 MHz, CD₃OD) δ 8.29 (d, J=8.6 Hz, 1H), 7.30 (d, J=5.2 Hz, 1H), 7.30 (m, 1H), 7.18 (d, J=5.2 Hz, 1H), 6.93 (d, J=8.7 Hz, 1H), 6.91 (m, 1H), 6.84 (m, 1H), 6.73 (d, J=8.7 Hz, 1H), 6.72 (d, J=8.6 Hz, 1H), 6.11 (s, 1H), 6.08 (s, 1H), 5.42 (s, 1H), 3.75 (s, 3H), 1.83 (s, 3H), 1.30 (s, 6H).

### Example 188

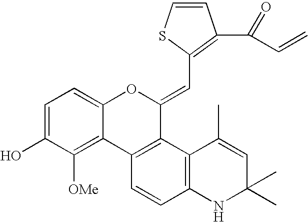

(Z)-5-(2′-(3′-Acryloylthienylmethylidene))1,2-dihydro-9-hydroxy-10-methoxy-2,2,4-trimethyl-5H-chromeno[3,4-f]quinoline (Compound 213, Structure 54 of Scheme XIV, where X═S, R¹³═H, R^{A}=vinyl).

This compound was prepared according to General Method 8A (EXAMPLE 152) from (Z)-5-(2′-(3′-formylthienylmethylidene))-1,2-dihydro-10-methoxy-2,2,4-trimethyl-9-(triisopropylsilyl)oxy-5H-chromeno[3,4-f]quinoline (EXAMPLE 167) and vinyl magnesium bromide, followed by treatment according to General Method 11 (EXAMPLE 167), followed by treatment according to General Method 12 (EXAMPLE 167) to afford Compound 213. ¹H NMR (500 MHz, CD₃OD) δ 8.37 (d, J=8.7 Hz, 1H), 7.49 (d, J=5.6 Hz, 1H), 7.33 (d, J=5.6 Hz, 1H), 7.28 (s, 1H), 7.11 (dd, J=17.0, 10.5 Hz, 1H), 7.01 (d, J=8.9 Hz, 1H), 6.80 (d, J=8.9 Hz, 1H), 6.77 (d, J=8.7 Hz, 1H), 6.28 (d, J=17.0 Hz, 1H), 5.85 (d, J=10.5 Hz, 1H), 5.55 (m, 1H), 3.77 (s, 3H), 2.03 (m, 3H), 1.34 (s, 6H).

### Example 189

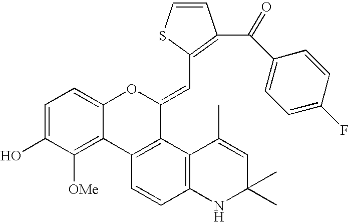

(Z)-5-(2′-(3′-(4″-Fluorobenzoyl)thienylmethylidene))1,2-dihydro-9-hydroxy-10-methoxy-2,2,4-trimethyl-5H-chromeno[3,4-f]quinoline (Compound 214, Structure 54 of Scheme XIV, where X═S, R¹³═H, R^{A}=4-fluorophenyl).

This compound was prepared according to General Method 8A (EXAMPLE 152) from (Z)-5-(2′-(3′-formylthienylmethylidene))-1,2-dihydro-10-methoxy-2,2,4-trimethyl-9-(triisopropylsilyl)oxy-5H-chromeno[3,4-f]quinoline (EXAMPLE 167) and 4-fluoromagnesium bromide, followed by treatment according to General Method 11 (EXAMPLE 167), followed by treatment according to General Method 12 (EXAMPLE 167) to afford Compound 214. ¹H NMR (500 MHz, CDCl₃) δ 8.22 (d, J=8.6 Hz, 1H), 7.81 (dd, J=8.5, 5.6 Hz, 2H), 7.23 (d, J=5.3 Hz, 1H), 7.13 (d, J=5.3 Hz, 1H), 7.10 (dd, J=8.5, 9.0 Hz, 2H), 7.09 (d, J=8.8 Hz, 1H), 6.89 (d, J=8.8 Hz, 1H), 6.71 (d, J=8.6 Hz, 1H), 6.59 (s, 1H), 5.59 (m, 1H), 4.27 (s, 1H), 3.78 (s, 3H), 2.08 (m, 3H), 1.37 (s, 6H).

### Example 190

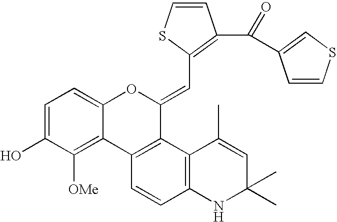

(Z)-5-(2′-(3′-(Thien-3″-ylcarbonyl)thienylmethylidene))1,2-dihydro-9-hydroxy-10-methoxy-2,2,4-trimethyl-5H-chromeno[3,4-f]quinoline (Compound 215, Structure 54 of Scheme XIV, where X═S, R¹³═H, R^{A}=3-thienyl).

This compound was prepared according to General Method 8A (EXAMPLE 152) from (Z)-5-(2′-(3′-formylthienylmethylidene))-1,2-dihydro-10-methoxy-2,2,4-trimethyl-9-(triisopropylsilyl)oxy-5H-chromeno[3,4-f]quinoline (EXAMPLE 167) and 3-thienylmagnesium iodide, followed by treatment according to General Method 11 (EXAMPLE 167), followed by treatment according to General Method 12 (EXAMPLE 167) to afford Compound 215. ¹H NMR (500 MHz, Acetone-d₆) δ 8.34 (d, J=8.6 Hz, 1H), 8.07 (d, J=2.7 Hz, 1H), 7.89 (s, 1H), 7.58 (dd, J=5.0, 2.7 Hz, 1H), 7.52 (d, J=5.0 Hz, 1H), 7.46 (d, J=5.3 Hz, 1H), 7.34 (d, J=5.3 Hz, 1H), 7.07 (d, J=8.7 Hz, 1H), 6.85 (s, 1H), 6.85 (d, J=8.7 Hz, 1H), 6.83 (d, J=8.6 Hz, 1H), 5.93 (s, 1H), 5.49 (m, 1H), 3.78 (s, 3H), 2.05 (m, 3H), 1.29 (s, 6H).

### Example 191

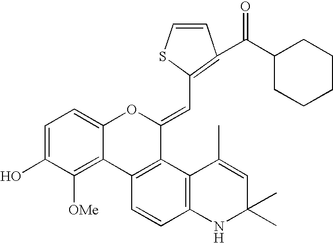

(Z)-(2′-(3″-(Cyclohexanecarbonyl)thienylmethylidene))1,2-dihydro-9-hydroxy-10-methoxy-2,2,4-trimethyl-5H-chromeno[3,4-f]quinoline (Compound 216, Structure 54 of Scheme XIV, where X═S, R¹³═H, R^{A}=cyclohexyl).

This compound was prepared according to General Method 8A (EXAMPLE 152) from (Z)-5-(2′-(3′-formylthienylmethylidene))-1,2-dihydro-10-methoxy-2,2,4-trimethyl-9-(triisopropylsilyl)oxy-5H-chromeno[3,4-f]quinoline (EXAMPLE 167) and cyclohexylmagnesium bromide, followed by treatment according to General Method 11 (EXAMPLE 167), followed by treatment according to General Method 12 (EXAMPLE 167) to afford Compound 216. ¹H NMR (300 MHz, CD₃OD) δ 8.35 (d, J=8.7 Hz, 1H), 7.45 (d, J=5.4 Hz, 1H), 7.31 (d, J=5.4 Hz, 1H), 7.17 (s, 1H), 6.99 (d, J=8.6 Hz, 1H), 6.78 (d, J=8.6 Hz, 1H), 6.76 (d, J=8.7 Hz, 1H), 5.54 (m, 1H), 3.76 (s, 3H), 3.09 (m, 1H), 2.01 (m, 3H), 1.89-1.65 (m, 4H), 1.47-1.37 (m, 4H), 1.33 (s, 6H), 1.31-1.22 (m, 2H).

### Example 192

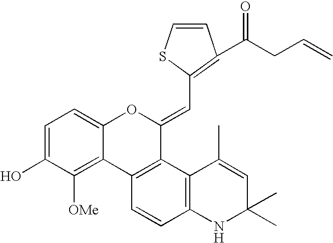

(Z)-5-(2′-(3′-(But-3″-enoyl)thienylmethylidene))1,2-dihydro-9-hydroxy-10-methoxy-2,2,4-trimethyl-5H-chromeno[3,4-f]quinoline (Compound 217, Structure 54 of Scheme XIV, where X═S, R¹³═H, R^{A}=allyl).

This compound was prepared according to General Method 8A (EXAMPLE 152) from (Z)-5-(2′-(3′-formylthienylmethylidene))-1,2-dihydro-10-methoxy-2,2,4-trimethyl-9-(triisopropylsilyl)oxy-5H-chromeno[3,4-f]quinoline and allyl magnesium bromide followed by treatment according to General Method 11 (EXAMPLE 167), followed by treatment according to General Method 12 (EXAMPLE 167) to afford Compound 217. ¹H NMR (300 MHz, CD₃OD) δ 8.36 (d, J=8.8 Hz, 1H), 7.53 (d, J=5.6 Hz, 1H), 7.38 (d, J=0.7 Hz, 1H), 7.31 (dd, J=5.6, 0.7 Hz, 1H), 7.00 (d, J=8.8 Hz, 1H), 6.80 (d, J=8.8 Hz, 1H), 6.77 (d, J=8.8 Hz, 1H), 6.02 (dd, J=17.1, 10.2 Hz, 1H), 5.55 (d, J=1.2 Hz, 1H), 5.22-5.13 (m, 2H), 3.77 (s, 3H), 3.66 (m, 2H), 2.02 (d, J=1.2 Hz, 3H), 1.34 (s, 6H).

### Example 193

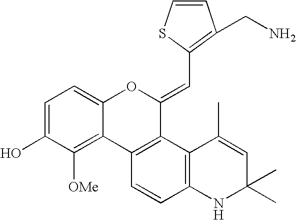

(Z)-5-(2′-(3′-(Aminomethyl)thienylmethylidene))1,2-dihydro-9-hydroxy-10-methoxy-2,2,4-trimethyl-5H-chromeno[3,4-f]quinoline (Compound 218, Structure 33 of Scheme IX, where X═S, R¹³═H, R¹⁹═H, R²⁰═H).

General Method 16: The product is light sensitive. Reductive amination of a carbonyl group to an amine using sodium cyanoborohydride. The amine (10 equiv.) was added to a solution of aldehyde (1 equiv.) in methanol (30 mL/mmol) at room temperature. The solution was allowed to warm to room temperature and stirred for 1 h. Methanol (30 mL/mmol), acetic acid (10 equiv.) and sodium cyanoborohydride (15 equiv.) were added sequentially and the solution stirred at room temperature for 0.2 h. In instances where the reaction was not complete, the reaction was allowed to proceed further. A saturated solution of ammonium chloride (200 mL/mmol) was added, ethyl acetate (200 mL/mmol) added and the layers separated. The aqueous layer was extracted with ethyl acetate (3×200 mL/mmol), the combined organics washed with a saturated solution of ammonium chloride (1000 mL/mmol), dried (Na₂SO₄) and concentrated under reduced pressure. Purification by flash chromatography, eluting with ethyl acetate:hexanes gave the corresponding amine.

This compound was prepared according to General Method 16 (EXAMPLE 193) from (Z)-5-(2′-(3′-formylthienylmethylidene))-1,2-dihydro-10-methoxy-2,2,4-trimethyl-9-(triisopropylsilyl)oxy-5H-chromeno[3,4-f]quinoline (EXAMPLE 167) and ammonium acetate, followed by treatment according to General Method 12 (EXAMPLE 167) to afford Compound 218. ¹H NMR (500 MHz, Acetone-d₆) δ 8.28 (d, J=8.8 Hz, 1H), 7.76 (s, 1H), 7.32 (d, J=5.3 Hz, 1H), 7.06 (d, J=5.3 Hz, 1H), 6.98 (d, J=8.8 Hz, 1H), 6.79 (d, J=8.8 Hz, 2H), 6.76 (d, J=8.8 Hz, 2H), 6.20 (s, 1H), 5.82 (br s, 1H), 5.41 (m, 1H), 3.81 (s, 2H), 3.76 (s, 3H), 1.99 (d, J=1.0 Hz, 3H), 1.24 (s, 6H).

### Example 194

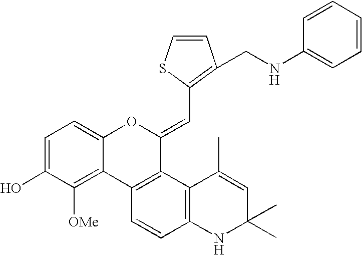

(Z)-5-(2′-(3′-(Phenylaminomethyl)thienylmethylidene))1,2-dihydro-9-hydroxy-10-methoxy-2,2,4-trimethyl-5H-chromeno[3,4-f]quinoline (Compound 219, Structure 33 of Scheme IX, where X═S, R¹³═H, R¹⁹═H, R²⁰=phenyl).

This compound was prepared according to General Method 16 (EXAMPLE 193) from (Z)-5-(2′-(3′-formylthienylmethylidene))-1,2-dihydro-10-methoxy-2,2,4-trimethyl-9-(triisopropylsilyl)oxy-5H-chromeno[3,4-f]quinoline (EXAMPLE 167) and aniline, followed by treatment according to General Method 12 (EXAMPLE 167) to afford Compound 219. ¹H NMR (500 MHz, Acetone-d₆) δ 8.29 (d, J=8.7 Hz, 1H), 7.82 (s, 1H), 7.33 (d, J=5.0 Hz, 1H), 7.11 (t, J=7.5 Hz, 2H), 7.06 (d, J=5.0 Hz, 1H), 6.99 (d, J=8.7 Hz, 1H), 6.81 (d, J=8.7 Hz, 1H), 6.76 (d, J=8.7 Hz, 1H), 6.69 (d, J=7.5 Hz, 2H), 6.60 (t, J=7.5 Hz, 1H), 6.21 (s, 1H), 5.81 (s, 1H), 5.30 (m, 1H), 5.03 (m, 1H), 4.26 (d, J=4.6 Hz, 2H), 3.76 (s, 3H), 2.04 (m, 3H), 1.15 (s, 6H).

### Example 195

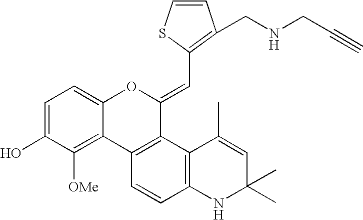

(Z)-5-(2′-(3′-(Prop-2″-ynylaminomethyl)thienylmethylidene))1,2-dihydro-9-hydroxy-10-methoxy-2,2,4-trimethyl-5H-chromeno[3,4-f]quinoline (Compound 220, Structure 33 of Scheme IX, where X═S, R³═H, R¹⁹═H, R²⁰=propyn-2-yl).

This compound was prepared according to General Method 16 (EXAMPLE 193) from (Z)-5-(2′-(3″-formylthienylmethylidene))-1,2-dihydro-10-methoxy-2,2,4-trimethyl-9-(triisopropylsilyl)oxy-5H-chromeno[3,4-f]quinoline (EXAMPLE 167) and propargylamine, followed by treatment according to General Method 12 (EXAMPLE 167) to afford Compound 220. ¹H NMR (500 MHz, Acetone-d₆) δ 8.30 (d, J=8.8 Hz, 1H), 7.32 (d, J=5.1, 0.6 Hz, 1H), 7.04 (d, J=5.1 Hz, 1H), 6.99 (d, J=8.8 Hz, 1H), 6.80 (d, J=8.8 Hz, 1H), 6.79 (d, J=8.8 Hz, 1H), 6.31 (d, J=0.6 Hz, 1H), 5.86 (s, 1H), 5.56 (q, J=1.2 Hz, 1H), 3.86 (s, 2H), 3.77 (s, 3H), 3.38 (d, J=2.4 Hz, 2H), 2.64 (t, J=2.4 Hz, 1H), 2.08 (d, J=1.2 Hz, 3H), 1.34 (s, 6H).

### Example 196

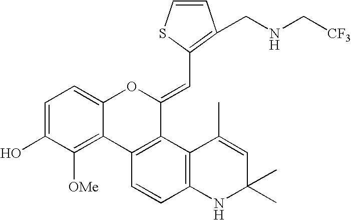

(Z)-5-(2′-(3′-((2″,2″,2″-Trifluoroethylamino)methyl)thienylmethylidene))1,2-dihydro-9-hydroxy-10-methoxy-2,2,4-trimethyl-5H-chromeno[3,4-f]quinoline ((Compound 221, Structure 33 of Scheme IX, where X═S, R¹³═H, R¹⁹═H, R²⁰=2,2,2-trifluoroethyl).

This compound was prepared according to General Method 16 (EXAMPLE 193) from (Z)-5-(2′-(3′-formylthienylmethylidene))-1,2-dihydro-10-methoxy-2,2,4-trimethyl-9-(triisopropylsilyl)oxy-5H-chromeno[3,4-f]quinoline (EXAMPLE 167) and 2,2,2-trifluoroethylamine, followed by treatment according to General Method 12 (EXAMPLE 167) to afford Compound 221. ¹H NMR (500 MHz, Acetone-d₆) δ 8.31 (d, J=8.8 Hz, 1H), 7.78 (s, 1H), 7.35 (dd, J=5.3, 0.6 Hz, 1H), 7.07 (d, J=5.3 Hz, 1H), 7.00 (d, J=8.8 Hz, 1H), 6.81 (d, J=8.8 Hz, 1H), 6.80 (d, J=8.8 Hz, 1H), 6.19 (d, J=0.6 Hz, 1H), 5.89 (s, 1H), 5.54 (q, J=1.2 Hz, 1H), 3.90 (m, 2H), 3.77 (s, 3H), 3.26 (m, 2H), 2.25 (m, 1H), 2.07 (d, J=1.2 Hz, 3H), 1.34 (s, 6H).

### Example 197

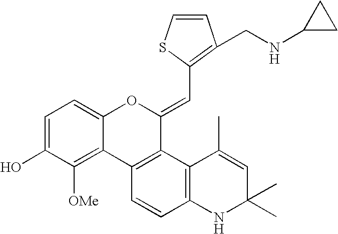

(Z)-5-(2′-(3′-(Cyclopropylaminomethyl)thienylmethylidene))1,2-dihydro-9-hydroxy-10-methoxy-2,2,4-trimethyl-5H-chromeno[3,4-f]quinoline (Compound 222, Structure 33 of Scheme IX, where X═S, R¹³═H, R¹⁹═H, R²⁰=cyclopropyl).

This compound was prepared according to General Method 16 (EXAMPLE 193) from (Z)-5-(2′-(3′-formylthienylmethylidene))-1,2-dihydro-10-methoxy-2,2,4-trimethyl-9-(triisopropylsilyl)oxy-5H-chromeno[3,4-f]quinoline (EXAMPLE 167) and cyclopropylamine, followed by treatment according to General Method 12 (EXAMPLE 167) to afford Compound 222. ¹H NMR (500 MHz, Acetone-d₆) δ 8.30 (d, J=8.8 Hz, 1H), 7.76 (s, 1H), 7.31 (dd, J=5.3, 0.5 Hz, 1H), 7.02 (d, J=5.3 Hz, 1H), 7.00 (d, J=8.7 Hz, 1H), 6.80 (d, J=8.7 Hz, 1H), 6.79 (d, J=8.7 Hz, 1H), 6.24 (d, J=0.5 Hz, 1H), 5.89 (s, 1H), 5.54 (q, J=1.3 Hz, 1H), 3.79 (s, 2H), 3.76 (s, 3H), 2.12 (m, 1H), 2.07 (d, J=1.3 Hz, 3H), 1.36 (s, 6H), 0.37 (m, 2H), 0.30 (m, 2H).

### Example 198

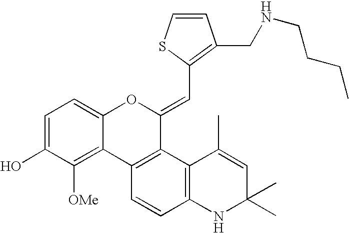

(Z)-5-(2′-(3′-(1″-Butylaminomethyl)thienylmethylidene))1,2-dihydro-9-hydroxy-10-methoxy-2,2,4-trimethyl-5H-chromeno[3,4-f]quinoline (Compound 223, Structure 33 of Scheme IX, where X═S, R¹³═H, R¹⁹═H, R²⁰=butyl).

This compound was prepared according to General Method 16 (EXAMPLE 193) from (Z)-5-(2′-(3′-formylthienylmethylidene))-1,2-dihydro-10-methoxy-2,2,4-trimethyl-9-(triisopropylsilyl)oxy-5H-chromeno[3,4-f]quinoline (EXAMPLE 167) and butylamine, followed by treatment according to General Method 12 (EXAMPLE 167) to afford Compound 223. ¹H NMR (500 MHz, Acetone-d₆) δ 8.32 (d, J=8.8 Hz, 1H), 7.35 (dd, J=5.2, 0.4 Hz, 1H), 7.24 (d, J=5.2 Hz, 1H), 6.99 (d, J=8.8 Hz, 1H), 6.82 (d, J=8.8 Hz, 1H), 6.81 (d, J=8.8 Hz, 1H), 6.21 (d, J=0.4 Hz, 1H), 5.93 (s, 1H), 5.54 (q, J=1.3 Hz, 1H), 3.88 (s, 2H), 3.77 (s, 3H), 2.71 (m, 2H), 2.08 (d, J=1.3 Hz, 3H), 1.58 (m, 2H), 1.36 (m, 2H), 1.35 (s, 6H), 0.89 (t, J=7.4 Hz, 3H).

### Example 199

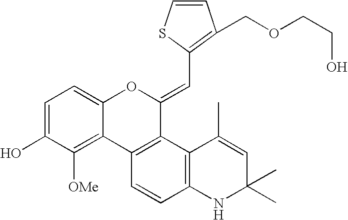

(Z)-5-(2′-(3′-(2″-Hydroxyethoxymethyl)thienylmethylidene))1,2-dihydro-9-hydroxy-10-methoxy-2,2,4-trimethyl-5H-chromeno[3,4-f]quinoline (Compound 224, Structure 17 of Scheme IV, where X═S, R¹³═H).

(Z)-5-(2′-(3′-(Ethoxycarbonylmethoxymethyl)thienylmethylidene))1,2-dihydro-10-methoxy-2,2,4-trimethyl-9-(triisopropylsilyl)oxy-5H-chromeno[3,4-f]quinoline (Structure 14A of Scheme IV, where X═S, PG=(triisopropyl)silyl, R¹³═H, R^{E}=Et) was prepared according to General Method 13 (EXAMPLE 170) from (Z)-5-(2′-(3′-(hydroxymethyl)thienylmethylidene))-1,2-dihydro-10-methoxy-2,2,4-trimethyl-9-(triisopropylsilyl)oxy-5H-chromeno[3,4-f]quinoline (EXAMPLE 167), potassium t-butoxide, and ethyl bromoacetate in THF to afford (Z)-5-(2′-(3′-(ethoxycarbonylmethoxymethyl)thienylmethylidene))1,2-dihydro-10-methoxy-2,2,4-trimethyl-9-(triisopropylsilyl)oxy-5H-chromeno[3,4-f]quinoline.

(Z)-5-(2′-(3′-(2″-Hydroxyethoxymethyl)thienylmethylidene))1,2-dihydro-9-hydroxy-10-methoxy-2,2,4-trimethyl-5H-chromeno[3,4-f]quinoline (Compound 224, Structure 17 of Scheme IV, where X═S, R¹³═H) was prepared in a manner similar to General Method 9 (EXAMPLE 155) except an ester, (Z)-5-(2′-(3′-(ethoxycarbonylmethoxymethyl)thienylmethylidene))1,2-dihydro-10-methoxy-2,2,4-trimethyl-9-(triisopropylsilyl)oxy-5H-chromeno[3,4-f]quinoline, was used in the reduction to afford the corresponding alcohol. This was followed by treatment according to General Method 12 (EXAMPLE 167) to afford Compound 224. ¹H NMR (500 MHz, Acetone-d₆) δ 8.31 (d, J=8.8 Hz, 1H), 7.79 (s, 1H), 7.34 (dd, J=5.2, 0.6 Hz, 1H), 7.03 (d, J=5.2 Hz, 1H), 7.00 (d, J=8.8 Hz, 1H), 6.81 (d, J=8.8 Hz, 1H), 6.80 (d, J=8.8 Hz, 1H), 6.20 (d, J=0.6 Hz, 1H), 5.90 (s, 1H), 5.55 (q, J=1.3 Hz, 1H), 4.52 (s, 2H), 3.77 (s, 3H), 3.66 (m, 2H), 3.58 (m, 1H), 3.54 (t, J=5.2 Hz, 2H), 2.07 (d, J=1.3 Hz, 3H), 1.34 (s, 6H).

### Example 200

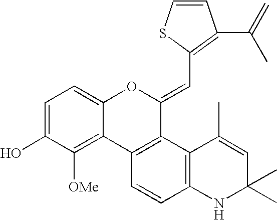

(Z)-5-(2′-(3′-Isopropenylthienylmethylidene))1,2-dihydro-9-hydroxy-10-methoxy-2,2,4-trimethyl-5H-chromeno[3,4-f]quinoline (Compound 225).

Tebbe reagent (10 equiv) was added to a solution of Compound 168 (EXAMPLE 152) in THF at 0° C. After 1 h, the solution was quenched with ether and the mixture filtered through Celite. Flash chromatography eluting with hexanes:ethyl acetate afforded Compound 225. ¹H NMR (500 MHz, CD₃OD) δ 8.29 (d, J=8.7 Hz, 1H), 7.27 (dd, J=5.2, 0.6 Hz, 1H), 6.95 (d, J=5.2 Hz, 1H), 6.93 (d, J=8.7 Hz, 1H), 6.73 (d, J=8.7 Hz, 2H), 6.19 (d, J=0.6 Hz, 1H), 5.48 (q, J=1.2 Hz, 1H), 5.16 (m, 1H), 4.86 (m, 1H), 3.76 (s, 3H), 2.03-2.02 (m, 3H), 2.00 (d, J=1.2 Hz, 3H), 1.27 (s, 6H).

### Example 201

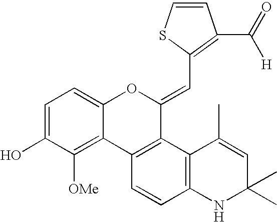

(Z)-5-(2′-(3′-Formylthienylmethylidene))1,2-dihydro-9-hydroxy-10-methoxy-2,2,4-trimethyl-5H-chromeno[3,4-f]quinoline (Compound 226).

This compound was prepared according to General Method 12 (EXAMPLE 167) from (Z)-5-(2′-(3′-formylthienylmethylidene))-1,2-dihydro-10-methoxy-2,2,4-trimethyl-9-(triisopropylsilyl)oxy-5H-chromeno[3,4-f]quinoline (EXAMPLE 167) to afford Compound 226. ¹H NMR (500 MHz, Acetone-d₆) δ 10.07 (s, 1H), 8.39 (d, J=8.7 Hz, 1H), 7.91 (s, 1H), 7.46 (d, J=5.4 Hz, 1H), 7.43 (dd, J=5.4, 0.7 Hz, 1H), 7.18 (d, J=0.7 Hz, 1H), 7.06 (d, J=8.7 Hz, 1H), 6.89 (d, J=8.7 Hz, 1H), 6.86 (d, J=8.7 Hz, 1H), 6.02 (s, 1H), 5.57 (m, 1H), 3.79 (s, 3H), 2.06 (d, J=1.2 Hz, 3H), 1.36 (s, 6H).

### Example 202

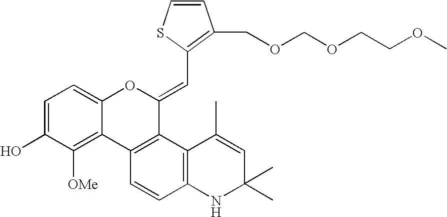

(Z)-5-(2′-(3′-(Methoxyethoxymethoxymethyl)thienylmethylidene))1,2-dihydro-9-hydroxy-10-methoxy-2,2,4-trimethyl-5H-chromeno[3,4-f]quinoline (Compound 227, Structure 15 of Scheme IV, where X═S, R¹³═H, R^{D}=methoxyethoxymethyl).

This compound was prepared according to General Method 13 (Example 170) from (Z)-5-(2′-(3′-hydroxymethylthienylmethylidene))-1,2-dihydro-10-methoxy-2,2,4-trimethyl-9-(triisopropylsilyl)oxy-5H-chromeno[3,4-f]quinoline (EXAMPLE 167), diisopropylethylamine, and MEM-Cl in dichloromethane, followed by treatment according to General Method 12 (EXAMPLE 167) to afford Compound 227. ¹H NMR (500 MHz, Acetone-d₆) δ 8.31 (d, J=8.7 Hz, 1H), 7.77 (s, 1H), 7.35 (d, J=5.1 Hz, 1H), 7.03 (d, J=5.1 Hz, 1H), 7.00 (d, J=8.7 Hz, 1H), 6.81 (d, J=8.7 Hz, 1H), 6.80 (d, J=8.7 Hz, 1H), 6.21 (s, 1H), 5.90 (s, 1H), 5.55 (q, J=1.2 Hz, 1H), 4.71 (s, 2H), 4.59 (s, 2H), 3.77 (s, 3H), 3.66 (m, 2H), 3.50 (m, 2H), 3.29 (s, 3H), 2.07 (d, J=1.2 Hz, 3H), 1.34 (s, 6H).

### Example 203

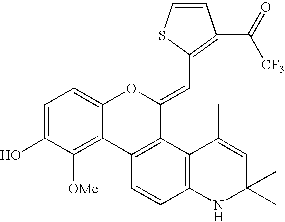

(Z)-5-(2′-(3′-(Trifluoroacetyl)thienylmethylidene))1,2-dihydro-9-hydroxy-10-methoxy-2,2,4-trimethyl-5H-chromeno[3,4-f]quinoline (Compound 228, Structure 54 of Scheme XIV, where X═S. R¹³═H, R^{A}=trifluoromethyl).

This compound was prepared according to General Method 11 (EXAMPLE 167) from (±)-(Z)-5-(2′-(3′-(1″-Hydroxy-2″,2″,2″-trifluoroethyl)thienylmethylidene))1,2-dihydro-10-methoxy-9-methoxymethoxy-2,2,4-trimethyl-5H-chromeno[3,4-f]quinoline (EXAMPLE 178), followed by treatment according to General Method 12 (EXAMPLE 167) to afford Compound 228. ¹H NMR (500 MHz, Acetone-d₆) δ 8.45 (d, J=8.8 Hz, 1H), 8.00 (s, 1H), 7.62 (s, 1H), 7.53 (d, J=5.7 Hz, 1H), 7.51 (dd, J=2.1, 5.7 Hz, 1H), 7.12 (d, J=8.8 Hz, 1H), 6.94 (d, J=8.8 Hz, 1H), 6.90 (d, J=8.8 Hz, 1H), 6.09 (s, 1H), 5.58 (q, J=1.2 Hz, 1H), 3.81 (s, 3H), 2.04 (m, 3H), 1.38 (s, 6H).

### Example 204

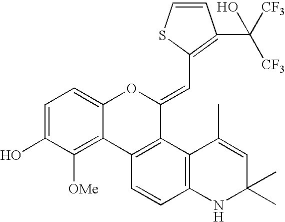

(Z)-5-(2′-(3′-(2″,2″,2″-Trifluoro-1″-hydroxy-1″-(trifluoromethyl)ethyl)thienylmethylidene))1,2-dihydro-9-hydroxy-10-methoxy-2,2,4-trimethyl-5H-chromeno[3,4-f]quinoline (Compound 229, Structure 8 of Scheme V, where X═S, R¹³═H, R^{A},R^{B}=trifluoromethyl).

(Z)-5-(2′-(3′-(Trifluoroacetyl)thienylmethylidene))1,2-dihydro-10-methoxy-9-methoxymethoxy-2,2,4-trimethyl-5H-chromeno[3,4-f]quinoline (Structure 21 of Scheme V, where X═S, R¹³═H, R^{A}=trifluoromethyl) was prepared according to General Method 11 (EXAMPLE 167) from (±)-(Z)-5-(2′-(3′-(1″-hydroxy-2″,2″,2″-trifluoroethyl)thienylmethylidene))1,2-dihydro-10-methoxy-9-methoxymethoxy-2,2,4-trimethyl-5H-chromeno[3,4-f]quinoline (EXAMPLE 178) to afford (Z)-5-(2′-(3″-(trifluoroacetyl)thienylmethylidene))1,2-dihydro-10-methoxy-9-methoxymethoxy-2,2,4-trimethyl-5H-chromeno[3,4-f]quinoline.

(Z)-5-(2′-(3′-(2″,2″,2″-Trifluoro-1″-hydroxy-1″-(trifluoromethyl)ethyl)thienylmethylidene))1,2-dihydro-9-hydroxy-10-methoxy-2,2,4-trimethyl-5H-chromeno[3,4-f]quinoline (Compound 229, Structure 8 of Scheme V, where X═S, R¹³═H, R^{A},R^{B}=trifluoromethyl) was prepared according to General Method 15 (EXAMPLE 178) from (Z)-5-(2′-(3′-(trifluoroacetyl)thienylmethylidene))1,2-dihydro-10-methoxy-9-methoxymethoxy-2,2,4-trimethyl-5H-chromeno[3,4-f]quinoline to afford (Z)-5-(2′-(3′-(2″,2″,2″-trifluoro-1″-hydroxy-1″-(trifluoromethyl)ethyl)thienylmethylidene))1,2-dihydro-10-methoxy-9-methoxymethoxy-2,2,4-trimethyl-5H-chromeno[3,4-f]quinoline.

This compound was then stirred in 10% HCl:methanol (10 mg starting material/1 mL solution) at rt for 3 h. The reaction was diluted with water, extracted with ethyl acetate, and the combined organic layer was washed sequentially with saturated sodium bicarbonate and saturated ammonium chloride, dried over sodium sulfate, and concentrated under reduced pressure. Flash chromatography (ethyl acetate:hexanes) afforded Compound 229. ¹H NMR (500 MHz, Acetone-d₆) δ 8.32 (d, J=8.8 Hz, 1H), 7.85 (s, 1H), 7.52 (dd, J=5.7, 0.6 Hz, 1H), 7.38 (s, 1H), 7.18 (d, J=5.7 Hz, 1H), 7.03 (d, J=0.6 Hz, 1H), 7.01 (d, J=8.8 Hz, 1H), 6.82 (d, J=8.8 Hz, 1H), 6.81 (d, J=8.8 Hz, 1H), 5.91 (s, 1H), 5.47 (q, J=1.5 Hz, 1H), 3.79 (s, 3H), 2.03 (d, J=1.5 Hz, 3H), 1.30 (s, 6H).

### Example 205

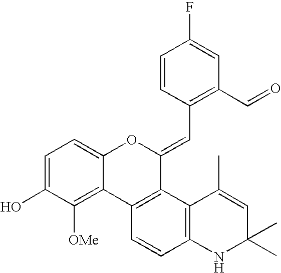

(Z)-5-(4′-Fluoro-2′-formylbenzylidene)1,2-dihydro-9-hydroxy-10-methoxy-2,2,4-trimethyl-5H-chromeno[3,4-f]quinoline (Compound 230).

This compound was prepared according to General Method 12 (EXAMPLE 167) from (Z)-5-(4′-fluoro-2′-formylbenzylidene)1,2-dihydro-10-methoxy-2,2,4-trimethyl-9-(triisopropylsilyl)oxy-5H-chromeno[3,4-f]quinoline (EXAMPLE 168) to afford Compound 230. ¹H NMR (500 MHz, CDCl₃) δ 10.19 (d, J=2.4 Hz, 1H), 8.21 (d, J=8.8 Hz, 1H), 8.02 (dd, J=8.6, 5.1 Hz, 1H), 7.56 (dd, J=8.8, 2.9 Hz, 1H), 7.31 (ddd, J=8.6, 8.0, 2.9 Hz, 1H), 6.79 (d, J=8.8 Hz, 1H), 6.73 (d, J=8.8 Hz, 1H), 6.71 (d, J=8.8 Hz, 1H), 6.35 (s, 1H), 5.58 (br s, 1H), 5.55 (d, J=1.3 Hz, 1H), 4.25 (s, 1H), 3.80 (s, 3H), 2.14 (d, J=1.3 Hz, 3H), 1.37 (s, 6H).

### Example 206

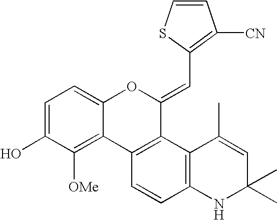

(Z)-5-(2′-(3′-Cyanothienylmethylidene))1,2-dihydro-9-hydroxy-10-methoxy-2,2,4-trimethyl-5H-chromeno[3,4-f]quinoline (Compound 231, Structure 28 of Scheme VIII, where X═S, R¹³═H).

A solution of (Z)-5-(2′-(3′-((E)-hydroxyiminomethyl)thienylmethylidene))-1,2-dihydro-10-methoxy-2,2,4-trimethyl-9-(triisopropylsilyl)oxy-5H-chromeno[3,4-f]quinoline (EXAMPLE 167) (25 mg, 0.04 mmol) in 2 mL of anhydrous THF was added 1,1′-carbonyldiimidazole (65 mg, 0.40 mmol) under nitrogen. The solution was heated to reflux for 2 hrs then allowed to cool to room temperature. The mixture was extracted with ethyl acetate (25 mL) and water. The organic layer was washed with brine, dried over magnesium sulfate, filtered, and concentrated. Flash chromatography (ethyl acetate:hexanes) afforded Compound 231. ¹H NMR (500 MHz, Acetone-d₆) δ 8.41 (d, J=8.7 Hz, 1H), 7.94 (s, 1H), 7.55 (dd, J=5.3, 0.7 Hz, 1H), 7.30 (d, J=5.3 Hz, 1H), 7.05 (d, J=8.7 Hz, 1H), 6.91 (d, J=8.7 Hz, 1H), 6.87 (d, J=8.7 Hz, 1H), 6.35 (d, J=0.7 Hz, 1H), 6.06 (s, 1H), 5.58 (q, J=1.3 Hz, 1H), 3.80 (s, 3H), 2.08 (d, J=1.3 Hz, 3H), 1.34 (s, 6H).

### Example 207

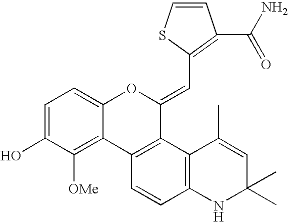

(Z)-5-(2′-(3′-Carbamoylthienylmethylidene))1,2-dihydro-9-hydroxy-10-methoxy-2,2,4-trimethyl-5H-chromeno[3,4-f]quinoline (Compound 232, Structure 30 of Scheme VIII, where X═S, R¹³═H, R¹⁹,R²⁰═H).

(Z)-5-(2′-(3′-Carboxythienylmethylidene))1,2-dihydro-9-hydroxy-10-methoxy-2,2,4-trimethyl-5H-chromeno[3,4-f]quinoline (Structure 29 of Scheme VIII, where X═S, R¹³═H).

To a solution of (Z)-5-(2′-(3′-cyanothienylmethylidene))1,2-dihydro-9-hydroxy-10-methoxy-2,2,4-trimethyl-5H-chromeno[3,4-f]quinoline (38 mg, 0.06 mmol) in 2 mL of ethylene glycol was added potassium hydroxide (23 mg, 0.41 mmol) and the mixture was heated to 175° C. in an oil bath. It was heated for 4 hrs then allowed to cool to room temperature and concentrated HCl was added to the solution at 0° C. until the pH=4-5. The mixture was extracted with ethyl acetate (25 mL) and water. The organic layer was washed with brine, dried over magnesium sulfate, filtered, and concentrated. Flash chromatography (1:1 hexanes:ethyl acetate) afforded 14 mg (51%) of (Z)-5-(2′-(3′-carboxythienylmethylidene))1,2-dihydro-9-hydroxy-10-methoxy-2,2,4-trimethyl-5H-chromeno[3,4-f]quinoline as a yellow solid.

(Z)-5-(2′-(3′-Carbamoylthienylmethylidene))1,2-dihydro-9-hydroxy-10-methoxy-2,2,4-trimethyl-5H-chromeno[3,4-f]quinoline (Compound 232, Structure 30 of Scheme VIII, where X═S, R¹³═H, R¹⁹,R²⁰═H). To a solution of the (Z)-5-(2′-(3′-carboxythienylmethylidene))1,2-dihydro-9-hydroxy-10-methoxy-2,2,4-trimethyl-5H-chromeno[3,4-f]quinoline (20 mg, 0.04 mmol) in 2 mL of anhydrous DMF was added 1-hydroxybenzotriazole hydrate (12 mg, 0.09 mmol), ammonium chloride (5 mg, 0.09 mmol), 1-(3-dimethylaminopropyl)-3-ethylcarbodiimide hydrochloride (17 mg, 0.09 mmol), and diisopropylamine (0.03 mL, 0.17 mmol). It was allowed to stir at room temperature for 14 hrs under nitrogen atmosphere. The mixture was then extracted with ethyl acetate (25 mL) and water. The organic layer was washed with brine, dried over magnesium sulfate, filtered, and concentrated. Flash chromatography (1:1 hexanes:ethyl acetate) afforded 12 mg (60%) of Compound 232. ¹H NMR (500 MHz, Acetone-d₆) δ 8.31 (d, J=8.7 Hz, 1H), 7.80 (s, 1H), 7.40 (d, J=5.4 Hz, 1H), 7.37 (dd, J=5.4, 0.6 Hz, 1H), 7.11 (s, 1H), 7.02 (d, J=8.7 Hz, 1H), 6.81 (d, J=8.7 Hz, 1H), 6.81 (d, J=8.7 Hz, 1H), 6.41 (s, 1H), 5.89 (s, 1H), 5.50 (q, J=1.2 Hz, 1H), 3.77 (s, 3H), 2.07 (d, J=1.2 Hz, 3H), 1.33 (s, 6H).

### Example 208

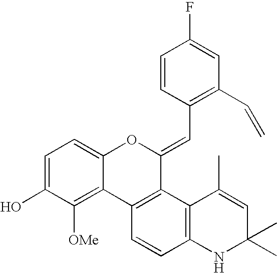

(Z)-5-(4′-Fluoro-2′-vinylbenzylidene)1,2-dihydro-9-hydroxy-10-methoxy-2,2,4-trimethyl-5H-chromeno[3,4-f]quinoline (Compound 233, Structure 47 of Scheme XII, where R³═H, R⁴═F, R⁵═H, R^{F}═H).

To a stirred suspension of methyl triphenylphosphonium bromide (150 mg, 0.42 mmol) in 5 mL of THF maintained at −78° C. was added a solution of n-butyllithium (0.3 mL, 1.4 M in hexanes, 0.42 mmol). The suspension was warmed to 0° C. and kept for 30 min before cooled back to −78° C. A solution of (Z)-5-(4′-fluoro-2′-formylbenzylidene)1,2-dihydro-10-methoxy-2,2,4-trimethyl-9-(triisopropylsilyl)oxy-5H-chromeno[3,4-f]quinoline (EXAMPLE 168) (40 mg, 0.065 mmol) in 2 mL of THF was added, and the resulting mixture was allowed to warm to rt and stirred overnight. The reaction was quenched with sat. NH₄Cl solution (10 mL), and extracted with EtOAc (3×15 mL). Combined organic layers were dried over anhydrous Na₂SO₄ and concentrated under reduced pressure. The residue was taken up in 5 mL of THF and was treated with TBAF (0.4 mL, 1 M solution in THF, 0.4 mmol) at rt. After 30 min, the reaction was quenched with sat. NH₄Cl solution (10 mL), and extracted with EtOAc (3×15 mL). Combined organic layers were dried over anhydrous Na₂SO₄ and concentrated under reduced pressure. The residue was purified by flash chromatography on silica gel (eluent: 20% ethyl acetate in hexanes) to afford Compound 233 as a colorless oil (3.4 mg, 11%). ¹H NMR (500 MHz, CDCl₃) δ 8.16 (d, J=8.5 Hz, 1H), 8.07 (dd, J=8.6, 6.1 Hz, 1H), 7.16 (dd, J=10.0, 2.8 Hz, 1H), 7.02 (td, J=8.6, 2.8 Hz, 1H), 6.87 (dd, J=17.4, 10.9 Hz, 1H), 6.79 (d, J=8.7 Hz, 1H), 6.76 (d, J=8.7 Hz, 1H), 6.69 (d, J=8.5 Hz, 1H), 5.83 (s, 1H), 5.59 (dd, J=17.4, 1.1 Hz, 1H), 5.56 (s, 1H), 5.51 (q, J=1.2 Hz, 1H), 5.28 (dd, J=10.9, 1.1 Hz, 1H), 4.20 (s, 1H), 3.79 (s, 3H), 2.11 (d, J=1.2 Hz, 3H), 1.35 (s, 6H).

### Example 209

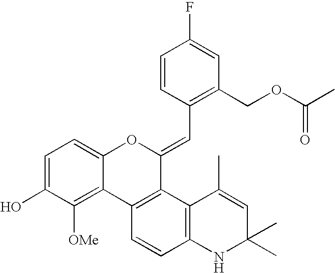

(Z)-5-(4′-Fluoro-2′-(acetoxymethyl)benzylidene)1,2-dihydro-9-hydroxy-10-methoxy-2,2,4-trimethyl-5H-chromeno[3,4-f]quinoline (Compound 234).

A solution of Compound 162 (EXAMPLE 146), acetic anhydride (4 equiv) in pyridine was stirred until consumption of starting material. The reaction was partitioned between ethyl acetate and dilute HCl. The reaction was washed with water, brine, dried over sodium sulfate, and concentrated. Flash chromatography (ethyl acetate:hexanes) afforded Compound 162. ¹H NMR (500 MHz, CDCl₃) δ 8.22 (dd, J=8.5, 5.9 Hz, 1H), 8.18 (d, J=8.5 Hz, 1H), 7.12-7.05 (m, 2H), 6.80 (d, J=8.8 Hz, 1H), 6.78 (d, J=8.8 Hz, 1H), 6.70 (d, J=8.5 Hz, 1H), 5.77 (s, 1H), 5.57 (s, 1H), 5.50 (m, 1H), 5.04 (s, 2H), 4.21 (s, 1H), 3.79 (s, 3H), 2.12 (d, J=1.2 Hz, 3H), 2.06 (s, 3H), 1.34 (s, 6H).

### Example 210

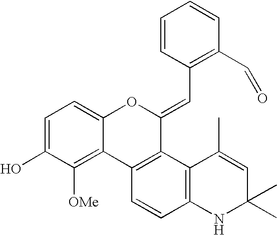

(Z)-5-(2′-Formylbenzylidine)-1,2-dihydro-9-hydroxy-10-methoxy-2,2,4-trimethyl-5H-chromeno[3,4-f]quinoline (Compound 235).

This compound was prepared according to General Method 12 (EXAMPLE 167) from (Z)-5-(2′-formylbenzylidene)1,2-dihydro-10-methoxy-2,2,4-trimethyl-9-(triisopropylsilyl)oxy-5H-chromeno[3,4-f]quinoline (EXAMPLE 169). ¹H NMR (500 MHz, CDCl₃) δ10.21 (s, 1H), 8.20 (d, J=8.6 Hz, 1H), 8.09 (d, J=7.7 Hz, 1H), 7.86 (d, J=7.7 Hz, 1H), 7.60 (t, J=7.7 Hz, 2H), 7.37 (t, J=7.7 Hz, 1H), 6.79 (d, J=8.8 Hz, 1H), 6.75 (d, J=8.8 Hz, 1H), 6.72 (d, J=8.6 Hz, 1H), 6.53 (s, 1H), 5.55 (q, J=1.2 Hz, 1H), 4.24 (s, 1H), 3.80 (s, 3H), 2.15 (d, J=1.2 Hz, 3H), 1.38 (s, 6H).

### Example 211

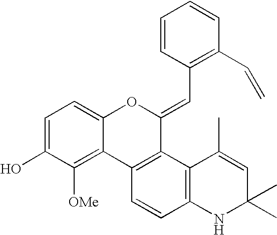

(Z)-5-(2′-Vinylbenzylidine)-1,2-dihydro-9-hydroxy-10-methoxy-2,2,4-trimethyl-5H-chromeno[3,4-f]quinoline (Compound 236, Structure 47 of Scheme XII, where R³═H, R⁴═H, R⁵═H, R^{F}═H).

This compound was prepared in a manner identical to Compound 233 (EXAMPLE 208) except (Z)-5-(2′-formylbenzylidene)1,2-dihydro-10-methoxy-2,2,4-trimethyl-9-(triisopropylsilyl)oxy-5H-chromeno[3,4-f]quinoline (EXAMPLE 169) was used as the starting material to afford Compound 236. ¹H NMR (500 MHz, CDCl₃) δ 8.16 (d, J=8.5 Hz, 1H), 8.10 (dd, J=7.9, 0.9 Hz, 1H), 7.47 (m, 1H), 7.32 (m, 1H), 7.21 (m, 1H), 6.93 (dd, J=17.5, 11.0 Hz, 1H), 6.79 (d, J=8.8 Hz, 1H), 6.79 (d, J=8.8 Hz, 1H), 6.69 (d, J=8.5 Hz, 1H), 5.92 (s, 1H), 5.58 (dd, J=17.5, 1.4 Hz, 1H), 5.50 (q, J=1.2 Hz, 1H), 5.23 (dd, J=11.0, 1.4 Hz, 1H), 4.19 (s, 1H), Peak15 2.13 (d, J=1.2 Hz, 3H), 1.35 (s, 6H).

### Example 212

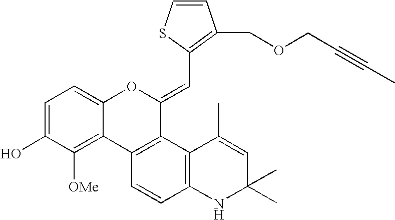

(Z)-5-(2′-(3′-(But-2″-ynloxymethyl)thienylmethylidene))1,2-dihydro-9-hydroxy-10-methoxy-2,2,4-trimethyl-5H-chromeno[3,4-f]quinoline (Compound 237, Structure 15 of Scheme IV, where X═S, R¹³═H, R^{D}=but-2-ynl).

This compound was prepared according to General Method 13 (Example 170) from (Z)-5-(2′-(3′-hydroxymethylthienylmethylidene))-1,2-dihydro-10-methoxy-2,2,4-trimethyl-9-(triisopropylsilyl)oxy-5H-chromeno[3,4-f]quinoline (EXAMPLE 167), 60% sodium hydride in mineral oil, and 1-bromo-2-butyne in THF, followed by treatment according to General Method 12 (EXAMPLE 167) to afford Compound 237. ¹H NMR (500 MHz, Acetone-d₆) δ 8.31 (d, J=8.8 Hz, 1H), 7.78 (s, 1H), 7.35 (d, J=5.3 Hz, 1H), 7.02 (d, J=5.3 Hz, 1H), 7.00 (d, J=8.8 Hz, 1H), 6.81 (d, J=8.8 Hz, 1H), 6.80 (d, J=8.8 Hz, 1H), 6.22 (s, 1H), 5.90 (s, 1H), 5.57 (m, 1H), 4.55 (s, 2H), 4.11 (s, 2H), 3.77 (s, 3H), 2.08 (m, 3H), 1.82 (s, 3H), 1.35 (s, 6H).

### Example 213

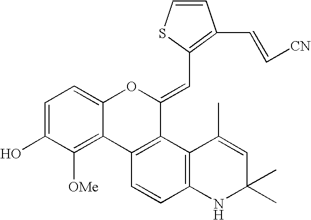

(Z)-5-(2′-(3′-(2″-(E)-Cyanovinyl)thienylmethylidene))1,2-dihydro-9-hydroxy-10-methoxy-2,2,4-trimethyl-5H-chromeno[3,4-f]quinoline (Compound 238, Structure 23 of Scheme VI, where X═S, R¹³═H, R^{F}═CN).

This compound was prepared in a manner similar to Compound 236 (EXAMPLE 211) except (Z)-5-(2′-(3′-formylthienylmethylidene))-1,2-dihydro-10-methoxy-2,2,4-trimethyl-9-(triisopropylsilyl)oxy-5H-chromeno[3,4-f]quinoline (EXAMPLE 167), diethyl(cyanomethyl)phosphonate and 60% NaH in mineral oil in THF was used to afford the cyanovinyl adduct. Subsequent treatment according to General Method 12 (EXAMPLE 167) afforded Compound 238. ¹H NMR (500 MHz, CDCl₃) δ 8.23 (d, J=8.5 Hz, 1H), 7.38 (d, J=16.2 Hz, 1H), 7.26 (d, J=5.4 Hz, 1H), 7.15 (dd, J=5.4, 0.6 Hz, 1H), 7.04 (d, J=8.8 Hz, 1H), 6.87 (d, J=8.8 Hz, 1H), 6.73 (d, J=8.5 Hz, 1H), 6.12 (d, J=0.6 Hz, 1H), 5.65 (d, J=16.2 Hz, 1H), Peak10 4.29 (s, 1H), 3.78 (s, 3H), 2.06 (d, J=1.0 Hz, 3H), 1.39 (s, 6H).

### Example 214

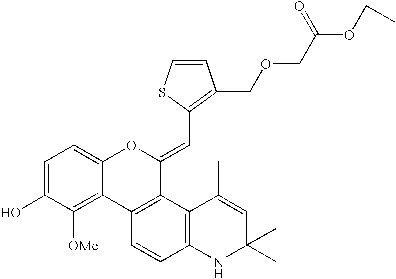

(Z)-5-(2′-(3′-(Ethoxycarbonylmethoxymethyl)thienylmethylidene))1,2-dihydro-9-hydroxy-10-methoxy-2,2,4-trimethyl-5H-chromeno[3,4-f]quinoline (Compound 239).

This compound was prepared according to General Method 12 (EXAMPLE 167) from (Z)-5-(2′-(3′-(ethoxycarbonylmethoxymethyl)thienylmethylidene))1,2-dihydro-10-methoxy-2,2,4-trimethyl-9-(triisopropylsilyl)oxy-5H-chromeno[3,4-f]quinoline (EXAMPLE 199) to afford Compound 239. ¹H NMR (500 MHz, Acetone-d₆) δ 8.31 (d, J=8.7 Hz, 1H), 7.79 (s, 1H), 7.36 (d, J=5.2 Hz, 1H), 7.04 (d, J=5.2 Hz, 1H), 7.01 (d, J=8.7 Hz, 1H), 6.81 (d, J=8.7 Hz, 1H), 6.81 (d, J=8.7 Hz, 1H), 6.24 (s, 1H), 5.90 (s, 1H), 5.53 (m, 1H), 4.63 (s, 2H), 4.15 (q, J=7.1 Hz, 2H), 4.10 (s, 2H), 3.77 (s, 3H), 2.05 (m, 3H), 1.35 (s, 6H), 1.22 (t, J=7.1 Hz, 3H).

### Example 215

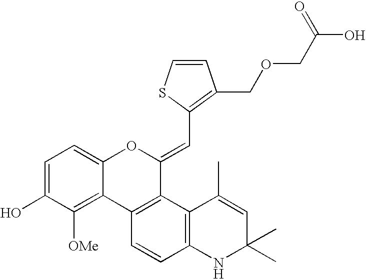

(Z)-5-(2′-(3′-(Carboxymethoxymethyl)thienylmethylidene))1,2-dihydro-9-hydroxy-10-methoxy-2,2,4-trimethyl-5H-chromeno[3,4-f]quinoline (Compound 240).

A solution of Compound 239 (EXAMPLE 214) and LiOH (3 equiv) in methanol was stirred at rt. The solution was quenched with saturated ammonium chloride and extracted with ethyl acetate. The organic layer was washed with brine, dried over sodium sulfate, filtered, and concentrated. Flash chromatography (ethyl acetate:hexanes) afforded Compound 240. ¹H NMR (300 MHz, Acetone-d₆) δ 8.31 (d, J=8.7 Hz, 1H), 7.36 (d, J=5.1 Hz, 1H), 7.05 (d, J=5.1 Hz, 1H), 7.01 (d, J=8.7 Hz, 1H), 6.81 (d, J=8.7 Hz, 1H), 6.81 (d, J=8.7 Hz, 1H), 6.24 (s, 1H), 5.89 (s, 1H), 5.55 (m, 1H), 4.65 (s, 2H), 4.13 (s, 2H), 3.77 (s, 3H), 2.06 (d, J=1.2 Hz, 3H), 1.34 (s, 6H).

### Example 216

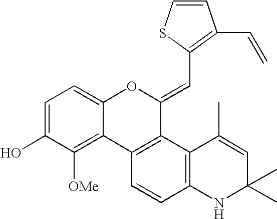

(Z)-5-(2′-(3′-Vinylthienylmethylidene))1,2-dihydro-9-hydroxy-10-methoxy-2,2,4-trimethyl-5H-chromeno[3,4-f]quinoline (Compound 241, Structure 23 of Scheme VI, where X═S, R¹³═H, R^{F}═H).

Tebbe reagent (10 equiv.) was added to a solution of (Z)-5-(2′-(3′-formylthienylmethylidene))-1,2-dihydro-10-methoxy-2,2,4-trimethyl-9-(triisopropylsilyl)oxy-5H-chromeno[3,4-f]quinoline (EXAMPLE 167) in THF at 0° C. After 1 h, the solution was quenched with ether and the mixture filtered through Celite. Flash chromatography eluting with hexanes:ethyl acetate afforded the olefinated product. Subsequent treatment according to General Method 12 (EXAMPLE 167) afforded Compound 241. ¹H NMR (300 MHz, CD₃OD) δ 8.32 (d, J=8.6 Hz, 1H), 7.27 (d, J=5.3 Hz, 1H), 7.22 (d, J=5.3 Hz, 1H), 6.94 (d, J=8.6 Hz, 1H), 6.76 (d, J=8.6 Hz, 1H), 6.75 (dd, J=17.4, 11.0 Hz, 1H), 6.74 (d, J=8.6 Hz, 1H), 6.13 (s, 1H), 5.60 (dd, J=17.4, 1.3 Hz, 1H), 5.54 (q, J=1.2 Hz, 1H), 5.20 (dd, J=11.0, 1.3 Hz, 1H), 3.76 (s, 3H), 2.03 (d, J=1.2 Hz, 3H), 1.32 (s, 6H).

### Example 217

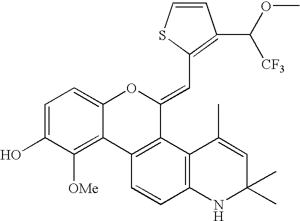

(±)-(Z)-5-(2′-(3′-(1″-Methoxy-2″,2″,2″-trifluoroethyl)thienylmethylidene))1,2-dihydro-9-hydroxy-10-methoxy-2,2,4-trimethyl-5H-chromeno[3,4-f]quinoline (Compound 242, Structure 26 of Scheme VII, where X═S, R¹³═H, R^{A}=trifluoromethyl, R^{B}═H, R=Me).

This compound was prepared according to General Method 13 (Example 170) from (±)-(Z)-5-(2′-(3′-(1″-Hydroxy-2″,2″,2″-trifluoroethyl)thienylmethylidene))1,2-dihydro-10-methoxy-9-methoxymethoxy-2,2,4-trimethyl-5H-chromeno[3,4-f]quinoline (EXAMPLE 178), NaH (60% mineral oil dispersion), and iodomethane in THF to afford the corresponding methyl ether. This compound was dissolved in 10% HCl:methanol (10 mg starting material/1 mL solution) and stirred at rt for 3 h. The reaction was diluted with water, extracted with ethyl acetate, and the combined organic layer was washed sequentially with saturated sodium bicarbonate and saturated ammonium chloride, dried over sodium sulfate, and concentrated under reduced pressure. Flash chromatography (ethyl acetate:hexanes) afforded Compound 242. ¹H NMR (300 MHz, Acetone-d₆) δ 8.34 (d, J=8.7 Hz, 1H), 7.85 (s, 1H), 7.49 (d, J=5.2 Hz, 1H), 7.10 (d, J=5.2 Hz, 1H), 7.02 (d, J=8.7 Hz, 1H), 6.83 (d, J=8.7 Hz, 1H), 6.83 (d, J=8.7 Hz, 1H), 6.26 (s, 1H), 5.96 (s, 1H), 5.56 (m, 1H), 4.95 (q, J=6.9 Hz, 1H), 3.79 (s, 3H), 3.38 (s, 3H), 2.05 (d, J=1.0 Hz, 3H), 1.35 (s, 3H), 1.32 (s, 3H).

### Example 218

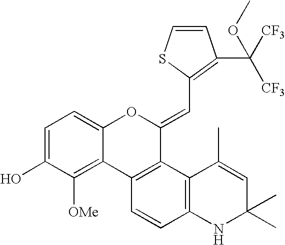

(Z)-5-(2′-(3′-(2″,2″,2″-Trifluoro-1″-methoxy-1″-trifluoromethyl)ethyl)thienylmethylidene))1,2-dihydro-9-hydroxy-10-methoxy-2,2,4-trimethyl-5H-chromeno[3,4-f]quinoline (Compound 243, Structure 26 of Scheme VII, X═S, R¹³═H, R^{B},R^{A}=trifluoromethyl, R^{G}=Me).

This compound was prepared according to General Method 13 (EXAMPLE 170) from (Z)-5-(2′-(3′-(2″,2″,2″-trifluoro-1-hydroxy-1″-trifluoromethyl)ethyl)thienylmethylidene))1,2-dihydro-10-methoxy-9-methoxymethoxy-2,2,4-trimethyl-5H-chromeno[3,4-f]quinoline (EXAMPLE 204), NaH (60% mineral oil dispersion, and iodomethane in THF to afford the corresponding methyl ether. This compound was then stirred in 10% HCl:methanol (10 mg starting material/1 mL solution) at rt for 3 h. The reaction was diluted with water, extracted with ethyl acetate, and the combined organic layer was washed sequentially with saturated sodium bicarbonate and saturated ammonium chloride, dried over sodium sulfate, and concentrated under reduced pressure. Flash chromatography (ethyl acetate:hexanes) afforded Compound 243. ¹H NMR (500 MHz, Acetone-d₆) δ 8.35 (d, J=8.8 Hz, 1H), 7.88 (s, 1H), 7.60 (dd, J=5.6, 0.7 Hz, 1H), 7.12 (d, J=5.6 Hz, 1H), 7.03 (d, J=8.8 Hz, 1H), 6.85 (d, J=8.8 Hz, 1H), 6.84 (d, J=8.8 Hz, 1H), 6.66 (d, J=0.7 Hz, 1H), 6.00 (s, 1H), 5.54 (q, J=1.2 Hz, 1H), 3.80 (s, 3H), 3.45 (s, 3H), 2.06 (d, J=1.2 Hz, 3H), 1.37 (s, 6H).

### Example 219

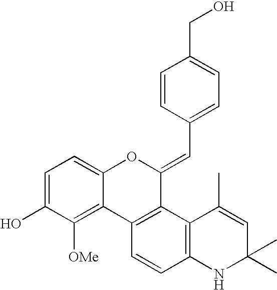

(Z)-5-(4′-Hydroxymethylbenzylidene)1,2-dihydro-9-hydroxy-10-methoxy-2,2,4-trimethyl-5H-chromeno[3,4-f]quinoline (Compound 244).

This compound was prepared according to General Method 4 (EXAMPLE 135) from 9-(tert-butyldimethylsilyl)oxy-10-methoxy-2,2,4-trimethyl-1,2-dihydro-5H-chromeno[3,4-f]quinoline-5-one and 4-(pyrrolidinecarbonyl)toluene. This product was then treated according to General Method 6 (EXAMPLE 141) to afford Compound 244. ¹H NMR (500 MHz, CDCl₃) δ 8.16 (d, J=8.6 Hz, 1H), 7.77 (d, J=8.3 Hz, 2H), 7.37 (d, J=8.3 Hz, 2H), 6.90 (d, J=8.8 Hz, 1H), 6.82 (d, J=8.8 Hz, 1H), 6.68 (d, J=8.6 Hz, 1H), 5.62 (s, 1H), 5.57 (s, 1H), 5.52 (q, J=1.3 Hz, 1H), 4.70 (s, 2H), 4.25 (s, 1H), 3.78 (s, 3H), 2.10 (d, J=1.3 Hz, 3H), 1.36 (s, 6H).

### Example 220

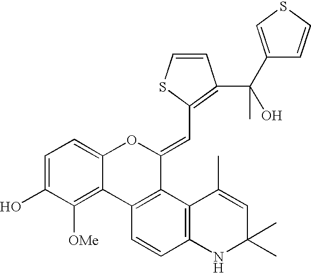

(Z)-5-(2′-(3′-(1″-Hydroxy-1″-(thien-3′″-yl)ethyl)thienylmethylidene))1,2-dihydro-9-hydroxy-10-methoxy-2,2,4-trimethyl-5H-chromeno[3,4-f]quinoline (Compound 245, Structure 8 of Scheme V, where X═S, R¹³═H, R^{B}=Me, R^{A}=3-thienyl).

This compound was prepared according to General Method 8A (EXAMPLE 152) from (Z)-5-(2′-(3′-formylthienylmethylidene))-1,2-dihydro-10-methoxy-2,2,4-trimethyl-9-(triisopropylsilyl)oxy-5H-chromeno[3,4-f]quinoline (EXAMPLE 167) and 3-thienylmagnesium iodide, followed by treatment according to General Method 11 (EXAMPLE 167), followed by treatment according to General Method 8 (EXAMPLE 152) using MeLi (1.6 M in ether), followed by treatment according to General Method 12 (EXAMPLE 167) to afford Compound 245. ¹H NMR (500 MHz, CD₃OD) δ 8.24 (d, J=8.6 Hz, 1H), 7.26-7.13 (m, 4H), 6.91 (d, J=8.7 Hz, 1H), 6.83 (d, J=5.0 Hz, 1H), 6.71-6.67 (m, 2H), 6.34 (s, 1H), 5.41 (m, 1H), 3.73 (s, 3H), 1.87 (d, J=1.0 Hz, 3H), 1.87 (s, 3H), 1.29 (s, 6H).

### Example 221

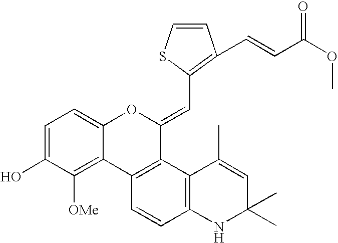

(Z)-5-(2′-(3′-(2″-Methoxycarbonylvinyl)thienylmethylidene))1,2-dihydro-9-hydroxy-10-methoxy-2,2,4-trimethyl-5H-chromeno[3,4-f]quinoline (Compound 246, Structure 23 of Scheme VI, where X═S, R¹³═H, R^{F}=methoxycarbonyl).

This compound was prepared in a manner similar to Compound 236 (EXAMPLE 211) except (Z)-5-(2′-(3′-formylthienylmethylidene))-1,2-dihydro-10-methoxy-2,2,4-trimethyl-9-(triisopropylsilyl)oxy-5H-chromeno[3,4-f]quinoline (EXAMPLE 167), methyl diethylphosphonoacetate, and 60% NaH in mineral oil in THF was used to afford the carbonyl adduct. Subsequent treatment according to General Method 12 (EXAMPLE 167) afforded Compound 246. ¹H NMR (300 MHz, CD₃OD) δ 8.37 (d, J=8.7 Hz, 1H), 7.79 (d, J=15.6 Hz, 1H), 7.38-7.31 (m, 2H), 6.96 (d, J=8.7 Hz, 1H), 6.80 (d, J=8.7 Hz, 1H), 6.77 (d, J=8.7 Hz, 1H), 6.31 (d, J=15.6 Hz, 1H), 6.27 (s, 1H), 5.60 (m, 1H), 3.78 (s, 3H), 3.77 (s, 3H), 2.03 (d, J=1.0 Hz, 3H), 1.35 (s, 6H).

### Example 222

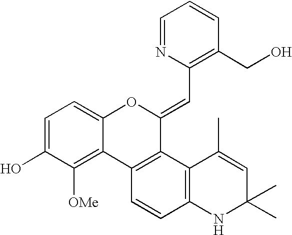

(Z)-5-(2′-(3′-Hydroxymethylpyridinylmethylidene))1,2-dihydro-9-hydroxy-10-methoxy-2,2,4-trimethyl-5H-chromeno[3,4-f]quinoline (Compound 247).

This compound was prepared according to General Method 4 (EXAMPLE 135) from 9-(tert-butyldimethylsilyl)oxy-10-methoxy-2,2,4-trimethyl-1,2-dihydro-5H-chromeno[3,4-f]quinoline-5-one and 2-methyl-3-(pyrrolidinecarbonyl)pyridine to afford (Z)-5-(2′-(3′-(Methoxycarbonyl)pyridinylmethylidene))1,2-dihydro-9-hydroxy-10-methoxy-2,2,4-trimethyl-5H-chromeno[3,4-f]quinoline. This compound was then treated according to General Method 6 (EXAMPLE 141) to afford Compound 247. ¹H NMR (300 MHz, CDCl₃) δ 8.57 (dd, J=4.8, 1.8 Hz, 1H), 8.20 (d, J=8.7 Hz, 1H), 7.88 (dd, J=7.7, 1.8 Hz, 1H), 7.19 (dd, J=7.7, 4.8 Hz, 1H), 6.72 (d, J=8.6 Hz, 1H), 6.71 (d, J=8.6 Hz, 1H), 6.63 (d, J=8.7 Hz, 1H), 5.95 (s, 1H), 5.78 (s, 1H), 5.51 (q, J=1.2 Hz, 1H), 4.79 (s, 2H), 4.22 (s, 1H), 3.76 (s, 3H), 2.24 (d, J=1.2 Hz, 3H), 1.32 (s, 6H).

### Example 223

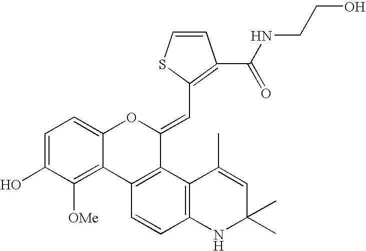

(Z)-5-(2′-(3′-(Hydroxyethylcarbamoyl)thienylmethylidene))1,2-dihydro-9-hydroxy-10-methoxy-2,2,4-trimethyl-5H-chromeno[3,4-f]quinoline (Compound 248, Structure 30 of Scheme VIII, where X═S, R¹³═H, R⁹═H, R²⁰=2-hydroxyethyl).

General Method 17: Preparation of an amide from a carboxylic acid. To a solution of the carboxylic acid (1 equiv) in 2 mL of anhydrous DMF was added 1-hydroxybenzotriazole hydrate (1.9 equiv), the amine (1.9 equiv), 1-(3-dimethylaminopropyl)-3-ethylcarbodiimide hydrochloride (1.9 equiv), and diisopropylamine (3.9 equiv). It was allowed to stir at room temperature for 14 hrs under nitrogen atmosphere. The mixture was then extracted with ethyl acetate (25 mL) and water. The organic layer was washed with brine, dried over magnesium sulfate, filtered, and concentrated. Flash chromatography (1:1 hexanes:ethyl acetate) afforded the desired amide.

(Z)-5-(2′-(3′-(Hydroxyethylcarbamoyl)thienylmethylidene))1,2-dihydro-9-hydroxy-10-methoxy-2,2,4-trimethyl-5H-chromeno[3,4-f]quinoline (Compound 248, Structure 30 of Scheme VIII, where X═S, R¹³═H, R¹⁹═H, R²⁰=2-hydroxyethyl) was prepared according to General Method 17 from (Z)-5-(2′-(3′-carboxythienylmethylidene))1,2-dihydro-9-hydroxy-10-methoxy-2,2,4-trimethyl-5H-chromeno[3,4-f]quinoline (EXAMPLE 207) and 2-hydroxyethylamine to afford Compound 248. ¹H NMR (500 MHz, Acetone-d₆) δ 8.31 (d, J=8.7 Hz, 1H), 7.80 (s, 1H), 7.48 (s, 1H), 7.37 (d, J=5.4 Hz, 1H), 7.33 (d, J=5.4 Hz, 1H), 7.18 (s, 1H), 7.01 (d, J=8.7 Hz, 1H), 6.83-6.80 (m, 2H), 5.90 (s, 1H), 5.53 (m, 1H), 4.02 (m, 1H), 3.77 (s, 3H), 3.65 (m, 2H), 3.45 (m, 2H), 2.07 (m, 3H), 1.34 (s, 6H).

### Example 224

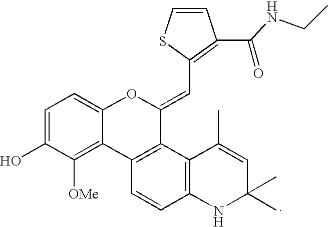

(Z)-5-(2′-(3′-Ethylcarbamoylthienylmethylidene))1,2-dihydro-9-hydroxy-10-methoxy-2,2,4-trimethyl-5H-chromeno[3,4-f]quinoline (Compound 249, Structure 30 of Scheme VIII, where X═S, R¹³═H, R⁹═H, R²⁰=ethyl).

This compound was prepared according to General Method 17 (EXAMPLE 223) from (Z)-5-(2′-(3′-carboxythienylmethylidene))1,2-dihydro-9-hydroxy-10-methoxy-2,2,4-trimethyl-5H-chromeno[3,4-f]quinoline (EXAMPLE 207) and ethylamine to afford Compound 249. ¹H NMR (500 MHz, Acetone-d₆) δ 8.31 (d, J=8.8 Hz, 1H), 7.79 (s, 1H), 7.37 (m, 1H), 7.36 (dd, J=5.4, 0.6 Hz, 1H), 7.28 (d, J=5.4 Hz, 1H), 7.12 (d, J=0.6 Hz, 1H), 7.01 (d, J=8.7 Hz, 1H), 6.81 (d, J=8.7 Hz, 1H), 6.81 (d, J=8.8 Hz, 1H), 5.52 (q, J=1.3 Hz, 1H), 3.77 (s, 3H), 3.34 (dq, J=5.7, 7.2 Hz, 2H), 2.07 (d, J=1.3 Hz, 3H), 1.34 (s, 6H), 1.15 (t, J=7.2 Hz, 3H).

### Example 225

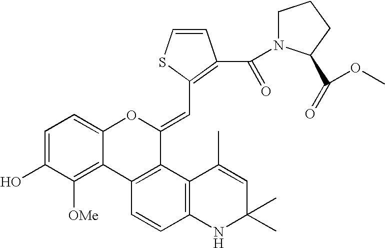

(Z)-5-(2′-(3′-((R)-2″-(Carbomethoxy)pyrrolidinecarbonyl)thienylmethylidene))1,2-dihydro-9-hydroxy-10-methoxy-2,2,4-trimethyl-5H-chromeno[3,4-f]quinoline (Compound 250, Structure 30 of Scheme VIII, where X═S, R¹³═H, NR¹⁹, R²⁰═(R)-2-(carbomethoxy)pyrrolidine).

This compound was prepared according to General Method 17 (EXAMPLE 223) from (Z)-5-(2′-(3′-carboxythienylmethylidene))1,2-dihydro-9-hydroxy-10-methoxy-2,2,4-trimethyl-5H-chromeno[3,4-f]quinoline (EXAMPLE 207) and 2-(carbomethoxy)pyrrolidine to afford Compound 250. ¹H NMR (500 MHz, Acetone-d₆) δ 8.30 (d, J=8.6 Hz, 1H), 7.81 (s, 1H), 7.44 (d, J=5.2 Hz, 1H), 7.11 (d, J=5.2 Hz, 1H), 7.02 (d, J=8.7 Hz, 1H), 6.82 (d, J=8.7 Hz, 1H>, 6.81 (d, J=8.6 Hz, 1H), 6.34 (s, 1H), 5.90 (s, 1H), 5.53 (m, 1H), 4.49 (dd, J=8.6, 4.1 Hz, 1H), 3.77 (s, 3H), 3.68 (s, 3H), 3.45 (m, 2H), 2.05 (s, 3H), 1.98-1.92 (m, 4H), 1.32 (s, 6H).

### Example 226

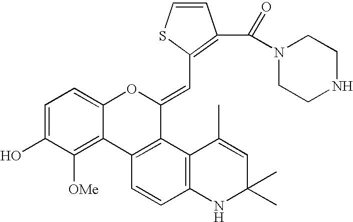

(Z)-5-(2′-(3′-(piperazinecarbonyl)thienylmethylidene))1,2-dihydro-9-hydroxy-10-methoxy-2,2,4-trimethyl-5H-chromeno[3,4-f]quinoline (Compound 251, Structure 30 of Scheme VIII, where X═S, R¹³═H, NR¹⁹R²⁰=piperazine).

This compound was prepared according to General Method 17 (EXAMPLE 223) from (Z)-5-(2′-(3′-carboxythienylmethylidene))1,2-dihydro-9-hydroxy-10-methoxy-2,2,4-trimethyl-5H-chromeno[3,4-f]quinoline (EXAMPLE 207) and piperazine to afford Compound 251. ¹H NMR (300 MHz, CD₃OD) δ 8.34 (d, J=8.7 Hz, 1H), 7.43 (d, J=5.2 Hz, 1H), 7.02 (d, J=5.2 Hz, 1H), 6.96 (d, J=8.7 Hz, 1H), 6.77 (d, J=8.7 Hz, 1H), 6.75 (d, J=8.7 Hz, 1H), 5.95 (s, 1H), 5.52 (m, 1H), 3.76 (s, 3H), 3.75-3.34 (m, 4H), 3.01-2.83 (m, 4H), 2.02 (m, 3H), 1.30 (s, 6H).

### Example 227

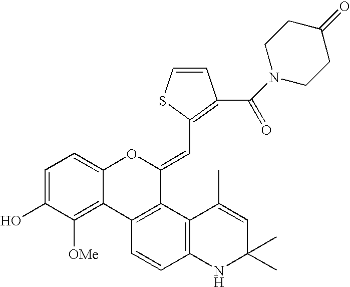

(Z)-5-(2′-(3′-(4″-Oxo-piperidinecarbonyl)thienylmethylidene))1,2-dihydro-9-hydroxy-10-methoxy-2,2,4-trimethyl-5H-chromeno[3,4-f]quinoline (Compound 252, Structure 30 of Scheme VIII, where X═S, R¹³═H, NR¹⁹R²⁰=4-oxo-piperidine).

This compound was prepared according to General Method 17 (EXAMPLE 223) from (Z)-5-(2′-(3′-carboxythienylmethylidene))1,2-dihydro-9-hydroxy-10-methoxy-2,2,4-trimethyl-5H-chromeno[3,4-f]quinoline (EXAMPLE 207) and 4-oxopiperidine to afford Compound 252. ¹H NMR (300 MHz, CD₃OD) δ 8.34 (d, J=8.7 Hz, 1H), 7.43 (d, J=5.3 Hz, 1H), 7.08 (d, J=5.3 Hz, 1H), 6.97 (d, J=8.7 Hz, 1H), 6.79-6.73 (m, 2H), 5.99 (s, 1H), 5.47 (m, 1H), 3.96 (m, 2H), 3.75 (s, 3H), 3.64 (m, 2H), 2.51 (m, 2H), 2.33 (m, 2H), 2.01 (s, 3H), 1.26 (s, 6H).

### Example 228

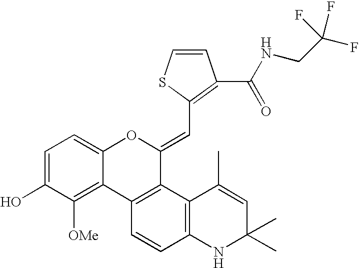

(Z)-5-(2′-(3′-(2″,2″,2″-Trifluoroethylcarbamoyl)thienylmethylidene))1,2-dihydro-9-hydroxy-10-methoxy-2,2,4-trimethyl-5H-chromeno[3,4-f]quinoline (Compound 253, Structure 30 of Scheme VIII, where X═S, R¹³═H, R¹⁹═H, R²⁰=2,2,2-trifluoroethyl).

This compound was prepared according to General Method 17 (EXAMPLE 223) from (Z)-5-(2′-(3′-carboxythienylmethylidene))1,2-dihydro-9-hydroxy-10-methoxy-2,2,4-trimethyl-5H-chromeno[3,4-f]quinoline (EXAMPLE 207) and 2,2,2-trifluoroethylamine. ¹H NMR (500 MHz, Acetone-d₆) δ 8.32 (d, J=8.7 Hz, 1H), 7.98 (t, J=6.2 Hz, 1H), 7.81 (s, 1H), 7.41 (dd, J=5.4, 0.6 Hz, 1H), 7.38 (d, J=5.4 Hz, 1H), 7.18 (d, J=0.6 Hz, 1H), 7.02 (d, J=8.7 Hz, 1H), 6.82 (d, J=8.7 Hz, 1H), 6.82 (d, J=8.7 Hz, 1H), 5.91 (s, 1H), 5.52 (q, J=1.3 Hz, 1H), 4.11 (dq, J=6.2, 9.4 Hz, 2H), 3.77 (s, 3H), 2.07 (d, J=1.3 Hz, 3H), 1.34 (s, 6H).

### Example 229

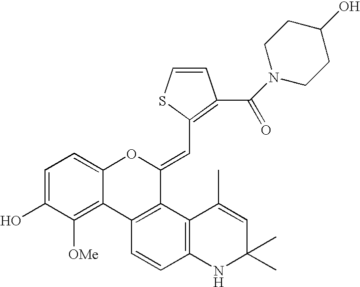

(Z)-5-(2′-(3′-(4″-Hydroxypiperidinecarbonyl)thienylmethylidene))1,2-dihydro-9-hydroxy-10-methoxy-2,2,4-trimethyl-5H-chromeno[3,4-f]quinoline (Compound 254, Structure 30 of Scheme VIII, where X═S, R¹³═H, NR¹⁹, R²⁰=4-hydroxypiperidine).

This compound was prepared according to Method 9 (EXAMPLE 155) from Compound 252 (EXAMPLE 227) to afford Compound 254. ¹H NMR (300 MHz, CD₃OD) δ 8.34 (d, J=8.7 Hz, 1H), 7.41 (d, J=5.3 Hz, 1H), 7.00-6.94 (m, 2H), 6.76 (d, J=8.7 Hz, 1H), 6.75 (d, J=8.7 Hz, 1H), 5.93 (s, 1H), 5.52 (m, 1H), 4.12 (m, 1H), 3.84 (m, 1H), 3.76 (s, 3H), 3.56 (m, 1H), 3.36 (m, 1H), 3.16 (m, 1H), 2.01 (m, 3H), 1.89 (m, 1H), 1.72 (m, 1H), 1.56 (m, 1H), 1.37 (m, 1H), 1.29 (s, 6H).

### Example 230

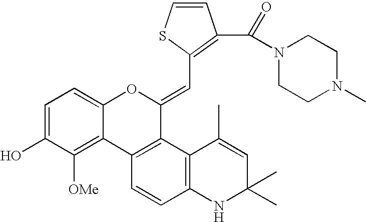

(Z)-5-(2′-(3′-(4″-Methylpiperazinecarbonyl)thienylmethylidene))1,2-dihydro-9-hydroxy-10-methoxy-2,2,4-trimethyl-5H-chromeno[3,4-f]quinoline (Compound 256, Structure 30 of Scheme VIII, where X═S, R¹³═H, NR¹⁹, R²⁰=4-methylpiperazine).

This compound was prepared according to General Method 17 (EXAMPLE 223) from (Z)-5-(2′-(3′-carboxythienylmethylidene))1,2-dihydro-9-hydroxy-10-methoxy-2,2,4-trimethyl-5H-chromeno[3,4-f]quinoline (EXAMPLE 207) and 4-methylpiperazine to afford Compound 256. ¹H NMR (300 MHz, Acetone-d₆) δ 8.33 (d, J=8.7 Hz, 1H), 8.12 (s, 1H), 7.45 (d, J=5.2 Hz, 1H), 7.02 (d, J=5.2 Hz, 1H), 7.01 (d, J=8.8 Hz, 1H), 6.82 (d, J=8.8 Hz, 1H), 6.82 (d, J=8.7 Hz, 1H), 6.07 (s, 1H), 5.94 (s, 1H), 5.59 (m, 1H), 3.77 (s, 3H), 3.77-3.37 (m, 4H), 2.66-2.51 (m, 4H), 2.38 (s, 3H), 2.05 (m, 3H), 1.34 (s, 6H).

### Example 231

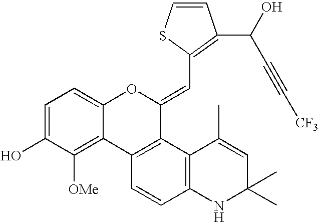

(±)-(Z)-5-(2′-(3′-(1″-Hydroxy-4″,4″,4″-trifluorobut-2″-ynyl)thienylmethylidene))1,2-dihydro-9-hydroxy-10-methoxy-2,2,4-trimethyl-5H-chromeno[3,4-f]quinoline (Compound 257, Structure 20 of Scheme V, where X═S, R¹³═H, R^{A}=3,3,3-trifluoropropynyl).

This compound was prepared according to General Method 8 (EXAMPLE 152) from (Z)-5-(2′-(3′-formylthienylmethylidene))-1,2-dihydro-10-methoxy-2,2,4-trimethyl-9-(triisopropylsilyl)oxy-5H-chromeno[3,4-f]quinoline (EXAMPLE 167) and lithium 3,3,3-trifluoropropynyl acetylide to afford the corresponding carbonyl adduct. The compound was then treated according to General Method 12 (EXAMPLE 167) to afford Compound 257. ¹H NMR (300 MHz, Acetone-d₆) δ 8.33 (d, J=8.6 Hz, 1H), 7.84 (s, 1H), 7.42 (d, J=5.3 Hz, 1H), 7.21 (d, J=5.3 Hz, 1H), 7.01 (d, J=8.7 Hz, 1H), 6.82 (d, J=8.7 Hz, 1H), 6.82 (d, J=8.6 Hz, 1H), 6.30 (s, 1H), 5.94 (s, 1H), 5.85 (s, 1H), 5.55 (s, 1H), 5.52 (m, 1H), 3.77 (s, 3H), 2.05 (m, 3H), 1.34 (s, 6H).

### Example 232

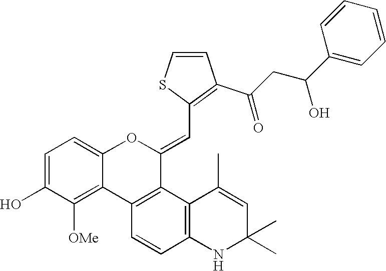

(Z)-5-(2′-(3′-(3″-Hydroxy-3″-phenylpropanoyl)thienylmethylidene))1,2-dihydro-9-hydroxy-10-methoxy-2,2,4-trimethyl-5H-chromeno[3,4-f]quinoline (Compound 258, Structure 53 of Scheme XIII, where X═S, R¹³═H, R^{G}=Ph).

(Z)-5-(2′-(3′-Acetylthienylmethylidene))1,2-dihydro-10-methoxy-2,2,4-trimethyl-9-(triisopropysilyl)oxy-5H-chromeno[3,4-f]quinoline (Structure 49 of Scheme XIII, where X═S, R¹³═H, PG=triisopropysilyl). This compound was prepared according to General Method 3 (EXAMPLE 135) from Compound 168 (EXAMPLE 152) to afford (Z)-5-(2′-(3′-acetylthienylmethylidene))1,2-dihydro-10-methoxy-2,2,4-trimethyl-9-(triisopropysilyl)oxy-5H-chromeno[3,4-f]quinoline.

(Z)-5-(2′-(3′-(3″-Hydroxy-3″-phenylpropanoyl)thienylmethylidene))1,2-dihydro-10-methoxy-9-(triisopropysilyl)oxy-2,2,4-trimethyl-5H-chromeno[3,4-f]quinoline (Structure 50 of Scheme XIII, where X═S, R¹³═H, R^{G}=Ph). Lithium bis(trimethylsilyl)amide (5 equiv, THF solution) was added to a solution of (Z)-5-(2′-(3′-acetylthienylmethylidene))1,2-dihydro-10-methoxy-2,2,4-trimethyl-9-(triisopropysilyl)oxy-5H-chromeno[3,4-f]quinoline and benzaldehyde in THF at 0° C. The reaction was quenched with saturated ammonium chloride, and extracted with ethyl acetate. The organic layer was washed with brine, dried over sodium sulfate, filtered, and concentrated. Flash chromatography (ethyl acetate:hexanes) afforded (Z)-5-(2′-(3′-(3″-hydroxy-3″-phenylbutanoyl)thienylmethylidene))1,2-dihydro-10-methoxy-9-(triisopropysilyl)oxy-2,2,4-trimethyl-5H-chromeno[3,4-f]quinoline.

(Z)-5-(2′-(3′-(3″-Hydroxy-3″-phenylpropanoyl)thienylmethylidene))1,2-dihydro-9-hydroxy-10-methoxy-2,2,4-trimethyl-5H-chromeno[3,4-f]quinoline (Compound 258, Structure 53 of Scheme XIII, where X═S, R¹³═H, R^{G}=Ph). This compound was prepared according to General Method 12 (EXAMPLE 167) to afford Compound 258. ¹H NMR (300 MHz, CD₃OD) δ 8.36 (d, J=8.7 Hz, 1H), 7.48 (d, J=5.4 Hz, 1H), 7.39 (m, 2H), 7.33-7.27 (m, 4H), 7.22 (tt, J=7.2, 1.4 Hz, 1H), 6.99 (d, J=8.7 Hz, 1H), 6.79 (d, J=8.7 Hz, 1H), 6.76 (d, J=8.7 Hz, 1H), 5.48 (q, J=1.2 Hz, 1H), 5.24 (dd, J=8.4, 4.8 Hz, 1H), 3.77 (s, 3H), 3.35 (dd, J=15.5, 8.4 Hz, 1H), 3.15 (dd, J=15.5, 4.8 Hz, 1H), 2.00 (d, J=1.2 Hz, 3H), 1.33 (s, 6H).

### Example 233

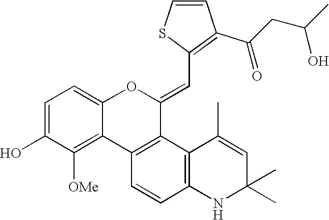

(Z)-5-(2′-(3′-(3″-Hydroxybutanoyl)thienylmethylidene))1,2-dihydro-9-hydroxy-10-methoxy-2,2,4-trimethyl-5H-chromeno[3,4-f]quinoline (Compound 259, Structure 53 of Scheme XIII, where X═S, R¹³═H, R^{G}=Me).

Lithium bis(trimethylsilyl)amide (5 equiv, THF solution) was added to a solution of (Z)-5-(2′-(3′-acetylthienylmethylidene))1,2-dihydro-10-methoxy-2,2,4-trimethyl-9-(triisopropysilyl)oxy-5H-chromeno[3,4-f]quinoline (EXAMPLE 233) and acetaldehyde in THF at 0° C. The reaction was quenched with saturated ammonium chloride, and extracted with ethyl acetate. The organic layer was washed with brine, dried over sodium sulfate, filtered, and concentrated. Flash chromatography (ethyl acetate:hexanes) afforded (Z)-5-(2′-(3′-(3″-hydroxybutanoyl)thienylmethylidene))1,2-dihydro-10-methoxy-9-(triisopropysilyl)oxy-2,2,4-trimethyl-5H-chromeno[3,4-f]quinoline.

(Z)-5-(2′-(3′-(3″-Hydroxybutanoyl)thienylmethylidene))1,2-dihydro-9-hydroxy-10-methoxy-2,2,4-trimethyl-5H-chromeno[3,4-f]quinoline (Compound 259, Structure 53 of Scheme XIII, where X═S, R¹³═H, R^{G}=Me) was prepared according to General Method 12 (EXAMPLE 167) from (Z)-5-(2′-(3′-(3″-hydroxybutanoyl)thienylmethylidene))1,2-dihydro-10-methoxy-9-(triisopropysilyl)oxy-2,2,4-trimethyl-5H-chromeno[3,4-f]quinoline to afford Compound 259. ¹H NMR (300 MHz, CD₃OD) δ 8.36 (d, J=8.7 Hz, 1H), 7.51 (d, J=5.5 Hz, 1H), 7.36 (d, J=0.7 Hz, 1H), 7.31 (dd, J=5.5, 0.7 Hz, 1H), 7.00 (d, J=8.6 Hz, 1H), 6.79 (d, J=8.6 Hz, 1H), 6.76 (d, J=8.7 Hz, 1H), 5.53 (q, J=1.1 Hz, 1H), 4.29 (m, 1H), 3.77 (s, 3H), 3.08 (dd, J=15.5, 7.3 Hz, 1H), 2.91 (dd, J=15.5, 5.4 Hz, 1H), 2.02 (d, J=1.1 Hz, 3H), 1.33 (s, 6H), 1.22 (d, J=6.3 Hz, 3H).

### Example 234

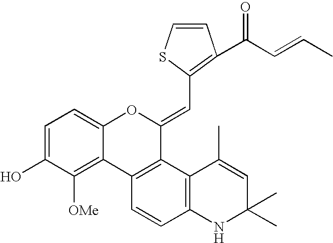

(Z)-5-(2′-(3′-(But-2″-enoyl)thienylmethylidene))1,2-dihydro-9-hydroxy-10-methoxy-2,2,4-trimethyl-5H-chromeno[3,4-f]quinoline (Compound 260, Structure 52 of Scheme XIII, where X═S, R¹³═H, R^{G}=Me).

A solution of (Z)-5-(2′-(3′-(3″-hydroxybutanoyl)thienylmethylidene))1,2-dihydro-10-methoxy-9-(triisopropysilyl)oxy-2,2,4-trimethyl-5H-chromeno[3,4-f]quinoline and p-toluenesulfonic acid (ca. 0.5 equiv) in toluene was stirred at approximately 40° C. After the starting material was consumed, the mixture was quenched with phosphate buffer and the aqueous layer was extracted with ethyl acetate. The organic layer was washed with brine, dried over sodium sulfate, filtered, and concentrated. Flash chromatography (ethyl acetate:hexanes) afforded the dehydrated product. The compound was then treated according to General Method 12 (EXAMPLE 167) to afford Compound 260. ¹H NMR (300 MHz, CD₃OD) δ 8.36 (d, J=8.7 Hz, 1H), 7.44 (d, J=5.5 Hz, 1H), 7.32 (dd, J=5.5, 0.7 Hz, 1H), 7.17 (d, J=0.7 Hz, 1H), 7.00 (d, J=8.7 Hz, 1H), 6.94 (dq, J=15.2, 6.6 Hz, 1H), 6.81 (dq, J=15.2, 1.3 Hz, 1H), 6.79 (d, J=8.7 Hz, 1H), 6.77 (d, J=8.7 Hz, 1H), 5.54 (q, J=1.2 Hz, 1H), 3.77 (s, 3H), 2.03 (d, J=1.2 Hz, 3H), 1.96 (dd, J=6.6, 1.3 Hz, 3H), 1.33 (s, 6H).

### Example 235

### Glucocorticoid Binding Assays

Preparation of Gr

A baculovirus expression plasmid containing cDNA encoding the human glucocorticoid receptor protein (GR) was prepared using standard techniques. See e.g., E. A. Allegretto et. al. 268 J. Biol. Chem., 26625 (1993); G. Srinivasan and B. Thompson, 4 Mol. Endo., 209 (1990); and D. R. O'Reilly et. al., in “Baculovirus Expression Vectors”, D. R. O'Reilly et. al., eds., W. H. Freeman, New York, N.Y., pp. 139-179 (1992). That expression plasmid was co-transfected together with wild type Autographa californica multiple nuclear polyhedrosis virus DNA into Spodopter frugiperda-21 (Sf-21) cells to generate recombinant virus containing GR cDNA. See e.g., O'Reilly, D. R., Miller, L. K., Luckow, V. A., Regulation of expression of a baculovirus ecdysteroid UDP glucosyltransferase gene. “Baculovirus Expression Vectors.” WH Freeman, NY, 139-179 (1992). That recombinant virus containing GR cDNA was collected.

A suspension culture of uninfected Sf21 cells was grown to a density of 1.2×10⁶ cells/ml and then infected with the recombinant virus containing GR cDNA at a multiplicity of infection of 2. Those infected Sf21 cells were incubated for 48 hours and then collected by centrifugation at 1000×g for 10 minutes at 4° C. The resulting cell pellets were resuspended in lysis buffer (50 mM Potassium Phosphate buffer, pH 7.0, 10 mM Monothioglycerol, 5 mM DTT, 20 mM Sodium Molybdate, 1 mM PMSF, 1 μg/mL aprotinin, and 10 μg/mL leupeptin) and incubated for 15 minutes on ice. Those resuspended cell pellets were homogenized using a Dounce homogenizer and a B pestle. A volume of 2 M KCl was added to the homogenized cell pellets to a final concentration of 0.4 M. The resulting GR lysates were centrifuged at 100,000×g for 60 min at 4° C. and stored for use in binding assays.

Binding Assays

Binding assay samples were prepared in separate mini-tubes in a 96-well format at 4° C. Each binding assay sample was prepared in a volume of 250 μL of Assay Buffer (10% glycerol, 25 mM sodium phosphate, 10 mM potassium fluoride, 10 mM sodium molybdate, 0.25 mM CHAPS, 2 mM DTT and 1 mM EDTA, (adjusted to pH 7.5)) containing 50 μg of GR lysate; 2-4 nM of [³H]dexamethasone at 84 Ci/mmol; and either a reference compound or a test compound. Test compounds included selective glucocorticoid binding compounds provided herein. Reference compounds were unlabeled dexamethasone and prednisone, which have been previously shown to bind to glucocorticoid receptors. Each reference compound and test compound was assayed at varying concentrations, ranging from 0 to 10⁻⁵ M. Each concentration of each reference compound and each test compound was assayed in triplicate. The assay samples were incubated for 16 hours at 4° C.

After incubation, 200 μL of 6.25% hydroxylapatite in assay buffer was added to each assay sample to precipitate the protein. The assay samples were then centrifuged and the supernatants were discarded. The resulting pellets were washed twice with assay buffer lacking DTT. Radioactivity in counts per minute (CPM) of each washed pellet was determined by liquid scintillation counter (MicroBeta™, Wallach).

Specific binding for a particular sample was calculated using the equation:
(Sample CPM)−(Average Non-specific CPM)

Average Non-specific CPM was defined as the amount of radioactivity from samples containing an excess (i.e. 1000 nM) of unlabeled dexamethasone. IC₅₀ values (the concentration of test compound required to decrease specific binding by 50%) were determined using the log-logit (Hill) method. Kᵢ values were determined using the Cheng-Prusoff equation using a previously determined K_{d} value for dexamethasone:
Kᵢ=IC₅₀/(1+[L]/K_{d})

[L]=concentration of labeled dexamethasone

K_{d}=dissociation constant of labeled dexamethasone

For a discussion of the calculation of Kᵢ, see e.g., Cheng, Y. C. and Prusoff, W. H. Biochem. Pharmacol. 22:3099 (1973). Kᵢ values for certain glucocorticoid binding compounds are shown in Table 1. The Ki values in Table 1 are provided as follows: A=<1 nM, B=1-2 nM, C=2-3 nM and D=>3 nM.

TABLE 1
Binding Data

| Compound | | |
|---|---|---|
| Number | Example | Ki (nM) |
| 11 | 1 | B |
| 12 | 2 | A |
| 14 | 4 | C |
| 15 | 5 | A |
| 18 | 8 | C |
| 22 | 12 | D |
| 29 | 19 | C |
| 37 | 27 | A |
| 63 | 49 | B |
| 67 | 53 | A |
| 75 | 60 | A |
| 86 | 71 | B |
| 90 | 75 | A |
| 97 | 82 | A |
| 103 | 88 | B |
| 107 | 92 | A |
| 111 | 96 | D |
| 132 | 117 | B |
| 134 | 118 | A |
| 138 | 122 | B |
| 143 | 127 | B |
| 146 | 130 | D |
| 151 | 135 | B |
| 154 | 138 | D |
| 157 | 141 | A |
| 159 | 143 | B |
| 166 | 150 | C |
| 167 | 151 | D |
| 171 | 155 | B |
| 178 | 160 | B |
| 179 | 160 | B |
| 193 | 170 | B |
| 200 | 177 | B |
| 201 | 177 | B |
| 202 | 178 | B |
| 215 | 190 | B |
| 222 | 197 | C |
| 237 | 212 | B |
| 249 | 224 | B |
| 252 | 227 | B |

Since modifications will be apparent to those of skill in this art, it is intended that the subject matter claimed herein be limited only by the scope of the appended claims.

1. A compound of Formula I: 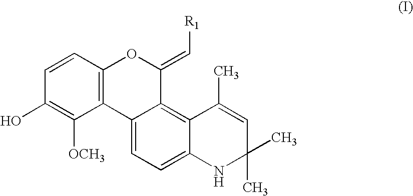
or a pharmaceutically acceptable derivative thereof, wherein:
R₁ is selected from among Formula II, III, and IV: 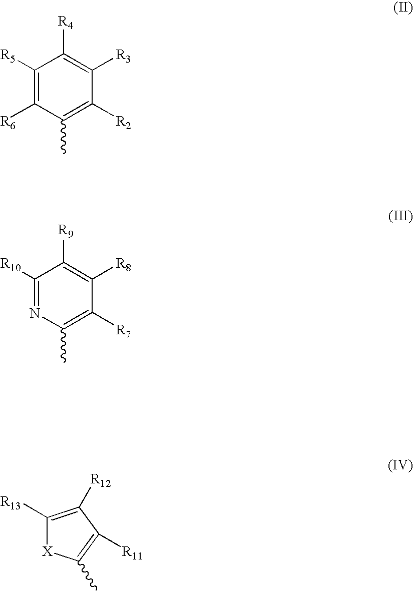
R₂ is selected from among hydrogen, F, Cl, Br, CN, an optionally substituted alkyl, an optionally substituted alkenyl, an optionally substituted alkynyl, an optionally substituted haloalkyl, an optionally substituted heteroalkyl, —CONR₁₄R₁₅, —OR₁₆, —COR₁₆, —SR₁₆, —SO₂NR₁₄R₁₅, an optionally substituted aryl, an optionally substituted heteroaryl, an optionally substituted heterocyclyl and an optionally substituted cycloalkyl;
R₃ is selected from among hydrogen, F, Cl, Br, CN, an optionally substituted alkyl, an optionally substituted alkenyl, an optionally substituted alkynyl, an optionally substituted haloalkyl, an optionally substituted heteroalkyl, —OR₁₆, —SR₁₆, an optionally substituted aryl, an optionally substituted heteroaryl, an optionally substituted heterocyclyl and an optionally substituted cycloalkyl;
R₄ is selected from among hydrogen, F, Cl, Br, CN, —OR₁₆, —SR₁₆, an optionally substituted alkyl, an optionally substituted alkenyl, an optionally substituted alkynyl, an optionally substituted haloalkyl, an optionally substituted heteroalkyl, an optionally substituted aryl, an optionally substituted heteroaryl, an optionally substituted heterocyclyl and an optionally substituted cycloalkyl; or
R₂ and R₃ together form an optionally substituted 5-6 member ring and R₄ is selected from among hydrogen, F, Cl, Br, CN, —OR₁₆, —SR₁₆, an optionally substituted alkyl, an optionally substituted alkenyl, an optionally substituted alkynyl, an optionally substituted haloalkyl, an optionally substituted heteroalkyl, an optionally substituted aryl, an optionally substituted heteroaryl, an optionally substituted heterocyclyl and an optionally substituted cycloalkyl; or
R₃ and R₄ together form an optionally substituted 4-6 member ring and R₂ is selected from among hydrogen, F, Cl, Br, CN, an optionally substituted alkyl, an optionally substituted alkenyl, an optionally substituted alkynyl, an optionally substituted haloalkyl, an optionally substituted heteroalkyl, —CONR₁₄R₁₅, —OR₁₆, —SR₁₆, —SO₂NR₁₄R₁₅, an optionally substituted aryl, an optionally substituted heteroaryl, an optionally substituted heterocyclyl and an optionally substituted cycloalkyl;
R₅ is selected from among hydrogen, F, Cl, Br, an optionally substituted alkyl, an optionally substituted alkenyl, an optionally substituted alkynyl, —SR₁₆ and —OR₁₆;
R₆ is selected from among hydrogen, F, Cl, Br, an optionally substituted alkyl, an optionally substituted alkenyl, an optionally substituted alkynyl;
R₇ is selected from among hydrogen, F, Cl, Br, CN, an optionally substituted alkyl, an optionally substituted alkenyl, an optionally substituted alkynyl, an optionally substituted haloalkyl, an optionally substituted heteroalkyl, —CONR₁₄R₁₅, —SO₂NR₁₄R₁₅, an optionally substituted aryl, an optionally substituted heteroaryl, an optionally substituted heterocyclyl and an optionally substituted cycloalkyl;
R₈ is selected from among hydrogen, F, Cl, Br, CN, an optionally substituted alkyl, an optionally substituted alkenyl, an optionally substituted alkynyl, an optionally substituted haloalkyl, an optionally substituted heteroalkyl, —OR₁₆, —SR₁₆, an optionally substituted aryl, an optionally substituted heteroaryl, an optionally substituted heterocyclyl and an optionally substituted cycloalkyl;
R₉ is selected from among hydrogen, F, Cl, Br, CN, an optionally substituted alkyl, an optionally substituted alkenyl, an optionally substituted alkynyl, an optionally substituted haloalkyl, and an optionally substituted heteroalkyl; or
R₇ and R₈ together form an optionally substituted 5-6 member ring and R₉ is selected from among hydrogen, F, Cl, Br, CN, an optionally substituted alkyl, an optionally substituted alkenyl, an optionally substituted alkynyl, an optionally substituted haloalkyl, and an optionally substituted heteroalkyl; or
R₈ and R₉ together form an optionally substituted 4-6 member ring and R₇ is selected from among hydrogen, F, Cl, Br, CN, an optionally substituted alkyl, an optionally substituted alkenyl, an optionally substituted alkynyl, an optionally substituted haloalkyl, an optionally substituted heteroalkyl, —CONR₁₄R₁₅, and an optionally substituted aryl;
R₁₀ is selected from among hydrogen, F, Cl, Br, an optionally substituted alkyl, an optionally substituted alkenyl, and an optionally substituted alkynyl; and
R₁₁ is selected from among hydrogen, F, Cl, Br, CN, an optionally substituted alkyl, an optionally substituted alkenyl, an optionally substituted alkynyl, an optionally substituted haloalkyl, an optionally substituted heteroalkyl, hydroxyiminoalkyl, alkoxyiminoalkyl, aryloxyiminoalkyl, —CONR₁₄R₁₅, SO₂NR₁₄R₁₅, OR₁₆, —SR₁₆, —COR₁₆, an optionally substituted aryl, an optionally substituted heteroaryl, an optionally substituted heterocyclyl and an optionally substituted cycloalkyl;
R₁₂ is selected from among hydrogen, F, Cl, Br, CN, an optionally substituted alkyl, an optionally substituted alkenyl, an optionally substituted alkynyl, an optionally substituted haloalkyl, an optionally substituted heteroalkyl, —OR₁₆, —SR₁₆, an optionally substituted aryl, an optionally substituted heteroaryl, an optionally substituted heterocyclyl and an optionally substituted cycloalkyl;
R₁₃ is selected from among hydrogen, F, Cl, Br, CN, CONR₁₄R₁₅, an optionally substituted alkyl, an optionally substituted alkenyl, an optionally substituted alkynyl, an optionally substituted haloalkyl, and an optionally substituted heteroalkyl; or
R₁₁ and R₁₂ together form an optionally substituted 5-6 member ring and R₁₃ is selected from among hydrogen, F, Cl, Br, CN, CONR₁₄R₁₅, an optionally substituted alkyl, an optionally substituted alkenyl, an optionally substituted alkynyl, an optionally substituted haloalkyl, and an optionally substituted heteroalkyl; or
R₁₂ and R₁₃ together form an optionally substituted 4-6 member ring and R₁₁ is selected from among hydrogen, F, Cl, Br, CN, an optionally substituted alkyl, an optionally substituted alkenyl, an optionally substituted alkynyl, an optionally substituted haloalkyl, an optionally substituted heteroalkyl, —CONR₁₄R₁₅, an optionally substituted aryl, an optionally substituted heteroaryl, an optionally substituted heterocyclyl and an optionally substituted cycloalkyl;
R₁₄ and R₁₅ are each independently selected from among hydrogen, an optionally substituted alkyl, an optionally substituted alkenyl, an optionally substituted alkynyl, an optionally substituted haloalkyl, an optionally substituted aryl, an optionally substituted heteroaryl, an optionally substituted heterocyclyl, an optionally substituted cycloalkyl and an optionally substituted heteroalkyl; or
R₁₄ and R₁₅ together form an optionally substituted 4-7 member ring;
R₁₆ is selected from among hydrogen, an optionally substituted alkyl, an optionally substituted alkenyl, an optionally substituted alkynyl, an optionally substituted haloalkyl, an optionally substituted heteroalkyl, an optionally substituted aryl, an optionally substituted heteroaryl, an optionally substituted heterocyclyl and an optionally substituted cycloalkyl;
X is selected from among O, S, and NR₁₇; and
R₁₇ is selected from among hydrogen, an optionally substituted alkyl, an optionally substituted alkenyl and an optionally substituted alkynyl;
wherein the substituents on the alkyl, alkenyl, alkynyl, aralkyl, aryl, heteroaryl, heterocyclyl, and cycloalkyl groups, when present, are each individually and independently selected from one to four group(s) selected from among: alkyl, alkenyl, alkynyl, cycloalkyl, aryl, heteroaryl, non-aromatic heterocycle, hydroxy, alkoxy, alkoxyalkoxy, aryloxy, mercapto, alkylthio, arylthio, cyano, halo, carbonyl, imino, hydroxyimino, alkoxyimino, aryloxyimino, aralkoxyiminothiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy and amino; including mono- and di-substituted amino groups, and the protected derivatives of amino groups;
wherein at least one position selected from among R₂, R₃, R₄, R₅, and R₆ is not hydrogen;
at least one position selected from among R₇, R₈, R₉, and R₁₀ is not hydrogen;
if R₄ is F, then at least one position selected from among R₂, R₃, R₅ and R₆ is not hydrogen;
if R₃ is F, then at least one position selected from among R₂, R₄, R₅, and R₆ is not hydrogen; and
if any two positions selected from among R₂, R₃, R₄, R₅, and R₆ are both F, then at least one of the other three positions selected from R₂, R₃, R₄, R₅, and R₆ is not hydrogen.

2. The compound of claim 1, wherein R₂ is selected from among hydrogen, F, Cl, Br, CN, an optionally substituted C₁-C₄ alkyl, an optionally substituted C₁-C₄ haloalkyl, an optionally substituted C₁-C₄ heteroalkyl, —CONR₁₄R₁₅, —OR₁₆, —SR₁₆, —SO₂NR₁₄R₁₅, and an optionally substituted aryl;
R₃ is selected from among hydrogen, F, Cl, Br, CN, an optionally substituted C₁-C₄ alkyl, an optionally substituted C₁-C₄ haloalkyl, an optionally substituted C₁-C₄ heteroalkyl, —OR₁₆, —SR₁₆ and an optionally substituted aryl; and
R₄ is selected from among hydrogen, F, Cl, Br, CN, —OR₁₆, a ring, an optionally substituted C₁-C₄ alkyl, an optionally substituted C₁-C₄ haloalkyl, and an optionally substituted C₁-C₄ heteroalkyl; or
R₂ and R₃ together form an optionally substituted 5-6 member ring and R₄ is selected from among hydrogen, F, Cl, Br, CN, —OR₁₆, a ring, an optionally substituted C₁-C₄ alkyl, an optionally substituted C₁-C₄ haloalkyl, and an optionally substituted C₁-C₄ heteroalkyl; or
R₃ and R₄ together form an optionally substituted 4-6 member ring and R₂ is selected from among hydrogen, F, Cl, Br, CN, an optionally substituted C₁-C₄ alkyl, an optionally substituted C₁-C₄ haloalkyl, an optionally substituted C₁-C₄ heteroalkyl, —CONR₁₄R₁₅, —OR₁₆, —SR₆, —SO₂NR₁₄R₁₅, and an optionally substituted aryl;
R₅ is selected from among hydrogen, F, Cl, Br, an optionally substituted C₁-C₄ alkyl, and OCH₃;
R₆ is selected from hydrogen and F;
R₇ is selected from among hydrogen, F, Cl, Br, CN, an optionally substituted C₁-C₄ alkyl, an optionally substituted C₁-C₄ haloalkyl, an optionally substituted C₁-C₄ heteroalkyl, —CONR₁₄R₁₅, and an optionally substituted aryl;
R₈ is selected from among hydrogen, F, Cl, Br, CN, an optionally substituted C₁-C₄ alkyl, an optionally substituted C₁-C₄ haloalkyl, an optionally substituted C₁-C₄ heteroalkyl, —OR₁₆, a phenyl that is optionally substituted with hydrogen, a halogen, an optionally substituted C₁-C₄ alkyl, an optionally substituted C₁-C₄ haloalkyl, and an optionally substituted C₁-C₄ heteroalkyl; and
R₉ is selected from among hydrogen, F, Cl, Br, CN, an optionally substituted C₁-C₄ alkyl, an optionally substituted C₁-C₄ haloalkyl, and an optionally substituted C₁-C₄ heteroalkyl; or
R₇ and R₈ together form an optionally substituted 5-6 member ring and R₉ is selected from among hydrogen, F, Cl, Br, CN, an optionally substituted C₁-C₄ alkyl, an optionally substituted C₁-C₄ haloalkyl, and an optionally substituted C₁-C₄ heteroalkyl; or
R₈ and R₉ together form an optionally substituted 4-6 member ring and R₇ is selected from among hydrogen, F, Cl, Br, CN, an optionally substituted C₁-C₄ alkyl, an optionally substituted C₁-C₄ haloalkyl, an optionally substituted C₁-C₄ heteroalkyl, —CONR₁₄R₁₅, and an optionally substituted aryl;
R₁₀ is selected from among hydrogen, F, Cl, CH₃, and OCH₃;
R₁₁ is selected from among hydrogen, F, Cl, Br, CN, an optionally substituted C₁-C₄ alkyl, an optionally substituted C₁-C₄ haloalkyl, an optionally substituted C₁-C₄ heteroalkyl, —CONR₁₄R₁₅, and an optionally substituted aryl;
R₁₂ is selected from among hydrogen, F, Cl, Br, CN, an optionally substituted C₁-C₄ alkyl, an optionally substituted C₁-C₄ haloalkyl, an optionally substituted C₁-C₄ heteroalkyl, —OR₁₆, a phenyl that is optionally substituted with hydrogen, a halogen, an optionally substituted C₁-C₄ alkyl, an optionally substituted C₁-C₄ haloalkyl, and an optionally substituted C₁-C₄ heteroalkyl; and
R₁₃ is selected from among hydrogen, F, Cl, Br, CN, CONR₁₄R₁₅, an optionally substituted C₁-C₄ alkyl, an optionally substituted C₁-C₄ haloalkyl, and an optionally substituted C₁-C₄ heteroalkyl; or
R₁₁ and R₁₂ together form an optionally substituted 5-6 member ring and R₁₃ is selected from among hydrogen, F, Cl, Br, CN, CONR₁₄R₁₅, an optionally substituted C₁-C₄ alkyl, an optionally substituted C₁-C₄ haloalkyl, and an optionally substituted C₁-C₄ heteroalkyl; or
R₁₂ and R₁₃ together form an optionally substituted 4-6 member ring and R₁₁, is selected from among hydrogen, F, Cl, Br, CN, CONR₁₄R₁₅, an optionally substituted C₁-C₄ alkyl, an optionally substituted C₁-C₄ haloalkyl, an optionally substituted C₁-C₄ heteroalkyl, —CONR₁₄R₁₅, and an optionally substituted aryl;
R₁₄ and R₁₅ are each independently selected from among hydrogen, an optionally substituted C₁-C₄ alkyl, an optionally substituted C₁-C₄ haloalkyl, and an optionally substituted C₁-C₄ heteroalkyl; or
R₁₄ and R₁₅ together form an optionally substituted 4-7 member ring;
R₁₆ is selected from among hydrogen, an optionally substituted C₁-C₄ alkyl, an optionally substituted C₁-C₄ haloalkyl, an optionally substituted C₁-C₄ heteroalkyl, and an optionally substituted aryl;
X is selected from among O, S, and NR₁₇;
R₁₇ is selected from among hydrogen and an optionally substituted C₁-C₄ alkyl; and
Wherein
at least one position selected from among R₂, R₃, R₄, R₅, and R₆ is not hydrogen;
at least one position selected from among R₇, R₈, R₉, and R₁₀ is not hydrogen;
if R₄ is F, then at least one position selected from among R₂, R₃, R₅ and R₆ is not hydrogen;
if R₃ is F, then at least one position selected from among R₂, R₄, R₅, and R₆ is not hydrogen; and
if any two positions selected from among R₂, R₃, R₄, R₅, and R₆ are both F, then at least one of the other three positions selected from among R₂, R₃, R₄, R₅, and R₆ is not hydrogen.

3. The compound of claim 1, wherein R₂ is selected from among hydrogen, halo, cyano, C₁-C₄ alkyl, C₂-C₄ alkenyl, aryl, haloalkoxy, haloalkylthio, formylaryl, hydroxyC₁-C₄alkyl, diC₁-C₄alkylaminoC₁-C₄alkyl, C₁-C₄alkylcarbonyl, hydroxyiminoC₁-C₄alkyl, alkoxyiminoC₁-C₄alkyl, alkoxyalkoxyC₁-C₄alkyl, hydroxyhaloC₂-C₄ alkyl, hydroxyhaloC₂-C₄ alkenyl, C₁-C₄alkylcarbonyloxyC₁-C₄alkyl, formyl, —OR₁₆, —SR₁₆, —CONR₁₄R₁₅, —SO₂NR₁₄R₁₅, wherein R₁₄ and R₁₅ are each independently selected from among hydrogen, C₁-C₄ alkyl, C₅-C₆ aryl C₁-C₄alkyl, C₃-C₇ cycloalkyl, or R₁₄ and R₁₅ together form an optionally substituted 4-7 member ring containing 1 or 2 heteroatoms selected from nitrogen and oxygen.

4. The compound of claim 1, wherein R₂ is selected from among hydrogen, F, Cl, Br, CN, an optionally substituted C₁-C₄ alkyl, an optionally substituted C₁-C₄ haloalkyl, an optionally substituted C₁-C₄ heteroalkyl, —CONR₁₄R₁₅, —OR₁₆, —SR₁₆, —SO₂NR₁₄R₁₅, and an optionally substituted aryl.

5. The compound of claim 1, wherein R₂ is phenyl.

6. The compound of claim 1, wherein R₂ is selected from among hydrogen, halo, cyano, C₁-C₄ alkyl, C₂-C₄ alkenyl, haloalkoxy, hydroxyC₁-C₄alkyl, alkoxyalkoxyC₁-C₄ alkyl, and hydroxyhaloC₁-C₄ alkyl.

7. The compound of claim 1, wherein R₂ is selected from among hydrogen, fluoro, chloro, bromo, cyano, methyl, vinyl, hydroxymethyl, diethylaminomethyl, methoxymethoxymethyl, hydroxyiminomethyl, acetyloxymethyl, 1-hydroxy-2-trifluoroethyl, phenyl, trifluoromethoxy, trifluoromethylthio, acetyl, formyl, diethylaminocarbonyl, 3-formylphenyl, N-benzyl-N-methylaminocarbonyl, dimethylaminocarbonyl, 1-pyrrolidinocarbonyl, 1-morpholinocarbonyl, 4-methyl piperazi-1-nocarbonyl, piperidinocarbonyl, N-cyclohexyl-N-methylaminocarbonyl, piperidinosulfonyl, and N,N-dimethylaminosulfonyl.

8. The compound of claim 1, wherein R₂ is selected from among hydrogen, fluoro, chloro, cyano, methyl, hydroxymethyl, methoxymethoxymethyl, 1-hydroxy-2-trifluoroethyl, vinyl and trifluoromethoxy.

9. The compound of claim 1, wherein R₃ is selected from among hydrogen, halo, hydroxy, C₁-C₄alkoxy, C₁-C₄alkyl, haloC₁-C₄alkyl, haloalkoxy, haloC₁-C₄alkylthio, aryl, heteroaryl, haloaryloxy, aryloxy, haloaryloxy, alkoxyaryloxy, C₁-C₄alkylaryloxy, haloalkoxyaryloxy, haloaryl and hydroxyC₁-C₄alkyl.

10. The compound of claim 1, wherein R₃ is selected from among hydrogen, F, Cl, Br, CN, an optionally substituted C₁-C₄ alkyl, an optionally substituted C₁-C₄ haloalkyl, an optionally substituted C₁-C₄ heteroalkyl, —OR₁₆, —SR₁₆ and an optionally substituted aryl.

11. The compound of claim 1, wherein R₃ is phenyl.

12. The compound of claim 1, wherein R₃ is selected from among hydrogen, halo, hydroxy, C₁-C₄alkoxy, C₁-C₄alkyl, haloC₁-C₄alkyl, haloalkoxy, haloC₁-C₄alkylthio, haloaryloxy, and aryloxy.

13. The compound of claim 1, wherein R₃ is selected from among hydrogen, fluoro, chloro, bromo, hydroxy, methoxy, methyl, tert-butyl, trifluoromethyl, hydroxymethyl, trifluoromethoxy, trifluoromethylthio, phenyl, 2,2-difluoro-1-ethoxy, 4,4,4-trifluoro-but-1-oxy, 2,4-difluorophenyl, 2-fluorophenyl, phenoxy, 3,6-dichlorophenoxy, 4-methoxyphenoxy, 3,4-dichloro-phenoxy, 4-methylphenoxy, 4-chlorophenoxy, 3-trifluoromethoxy-phenoxy, 4-fluorophenoxy, 3-thienyl, 3,3-difluoro-2,2,2-trifluoroprop-1-yloxy, 3,5-dichlorophenoxy, 4-fluorobenzyloxy, 3-fluorobenzyloxy and 3-pyridyl.

14. The compound of claim 13, wherein R₃ is selected from among hydrogen, fluoro, chloro, hydroxy, methyl, trifluoromethyl, hydroxymethyl, trifluoromethoxy, phenoxy, trifluoromethylthio and 4-fluorophenoxy.

15. The compound of claim 1, wherein R₄ is selected from among hydrogen, halo, hydroxy, C₁-C₄ alkyl, C₂-C₄alkenyl, C₃-C₆cycloalkyl, haloC₁-C₄ alkyl, aryl, hydroxy C₁-C₄alkyl, alkoxy, haloalkoxy, aralkoxy, haloaralkoxy, alkylaralkoxy, haloaryl, or R₃ and R₄ together form alkylenedioxy.

16. The compound of claim 1, wherein R₄ is selected from among hydrogen, chloro, bromo, hydroxy, methoxy, fluoro, trifluoromethoxy, methyl, ethyl, isopropyl, vinyl, benzyloxy, phenyl, cyclohexyl, trifluoromethyl, 4-methylbenzyloxy, hydroxymethyl, or R₃ and R₄ together form a methylenedioxy.

17. The compound of claim 1, wherein R₄ is selected from hydrogen and halogen.

18. The compound of claim 1, wherein R₄ is hydrogen.

19. The compound of claim 1, wherein R₄ is halogen.

20. The compound of claim 1, wherein R₄ is fluorine.

21. The compound of claim 1, wherein R₂ and R₃ together form an optionally substituted 5-6 member ring and R₄ is selected from among hydrogen, F, Cl, Br, CN, —OR₁₆, an optionally substituted C₁-C₄ alkyl, an optionally substituted C₁-C₄ haloalkyl, an optionally substituted C₁-C₄ heteroalkyl, an optionally substituted cycloalkyl, an optionally substituted heterocyclyl and an optionally substituted heteroaryl ring.

22. The compound of claim 1, wherein R₃ and R₄ together form an optionally substituted 4-6 member ring and R₂ is selected from among hydrogen, F, Cl, Br, CN, an optionally substituted C₁-C₄ alkyl, an optionally substituted C₁-C₄ haloalkyl, an optionally substituted C₁-C₄ heteroalkyl, —CONR₁₄R₁₅, —OR₆, —SR₁₆, —SO₂NR₁₄R₁₅, an optionally substituted cycloalkyl, an optionally substituted heterocyclyl and an optionally substituted heteroaryl ring.

23. The compound of claim 1, wherein R₂ and R₃ together form alkylenedioxy.

24. The compound of claim 1, wherein R₂ and R₃ together with the phenyl ring on which they are substituted form optionally substituted benzo-1,3-dioxan or optionally substituted naphthyl ring.

25. The compound of claim 1, wherein R₅ is selected from among hydrogen, halo, haloC₁-C₄alkyl, C₁-C₄alkyl, and C₁-C₄alkoxy.

26. The compound of claim 1, wherein R₅ is selected from among hydrogen, F, Cl, Br, optionally substituted C₁-C₄ alkyl, and OCH₃.

27. The compound of claim 1, wherein R₅ is selected from among hydrogen, F, Cl, Br, methyl, trifluoromethyl, isobutyl and methoxy.

28. The compound of claim 1, wherein R₅ is selected from among hydrogen, F, Cl and Br.

29. The compound of claim 1, wherein, R₅ is F.

30. The compound of claim 1, wherein R₅ is hydrogen.

31. The compound of claim 1, wherein R₆ is selected from among hydrogen, halo and C₁-C₄alkyl.

32. The compound of claim 1, wherein R₆ is selected from hydrogen and halo.

33. The compound of claim 1, wherein R₆ is selected from hydrogen and fluoro.

34. The compound of claim 1, wherein R₆ is hydrogen.

35. The compound of claim 1, wherein R₆ is fluoro.

36. The compound of claim 1, wherein at least one position selected from among R₂, R₃, R₄, R₅, and R₆ is not hydrogen.

37. The compound of claim 1, wherein at least one position selected from among R₇, R₈, R₉, and R₁₀ is not hydrogen.

38. The compound of claim 1, wherein if R₄ is F, then at least one position selected from among R₂, R₃, R₅ and R₆ is not hydrogen.

39. The compound of claim 1, wherein if R₃ is F, then at least one position selected from among R₂, R₄, R₅, and R₆ is not hydrogen.

40. The compound of claim 1, wherein if any two positions selected from among R₂, R₃, R₄, R₅, and R₆ are both F, then at least one of the other three positions selected from among R₂, R₃, R₄, R₅, and R₆ is not hydrogen.

41. The compound of claim 1, wherein R₁ is:

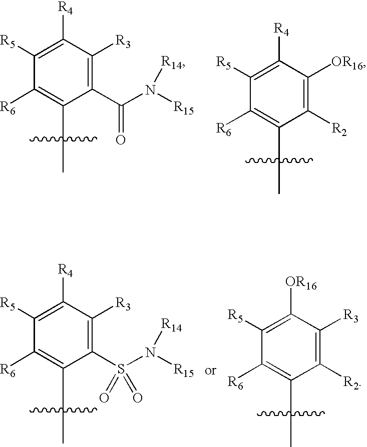

42. The compound of claim 1, wherein R₁ is

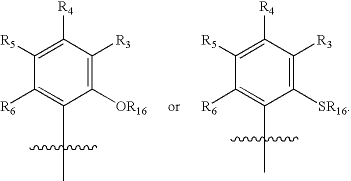

43. The compound of claim 42, wherein R₁₆ is hydrogen, optionally substituted C₁-C₄alkyl, haloC₁-C₄alkyl, optionally substituted aryl, haloaryloxy and C₁-C₄alkoxyC₁-C₄alkyl.

44. The compound of claim 1, wherein R₁ is:

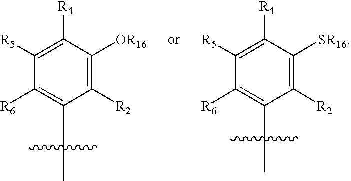

45. The compound of claim 44, wherein R₁₆ is hydrogen, methyl, methoxy, trifluoromethyl, 4-fluorophenyl, 4-methylbenzyl, 4,4,4-trifluorobutyl, 2-fluoroethyl, 3,3-difluoro-2,2,2-trifluoropropyl, 4-fluorobenzyl, 2-fluorobenzyl, 4-methoxyphenyl, 3,4-dichlorophenyl, 4-tolyl, 4-chlorophenyl, 3-trifluoromethoxyphenyl, and phenyl.

46. The compound of claim 1, wherein R₁ is

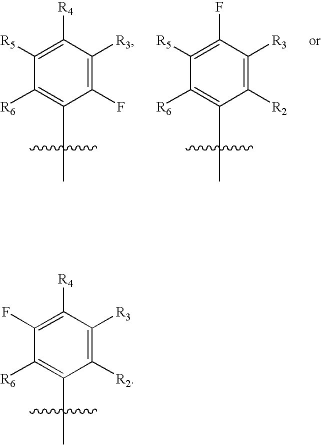

47. The compound of claim 1, wherein R₇ is selected from among hydrogen, F, Cl, Br, CN, an optionally substituted C₁-C₄ alkyl, an optionally substituted C₁-C₄ haloalkyl, an optionally substituted C₁-C₄ heteroalkyl, —CONR₁₄R₁₅, and an optionally substituted aryl.

48. The compound of claim 1, wherein R₇ is an optionally substituted aryl.

49. The compound of claim 1, wherein R₇ is an optionally substituted phenyl.

50. The compound of claim 49, wherein R₇ is substituted with one to three substituents selected from among hydrogen, halogen, C₁-C₄ alkyl, C₁-C₄ haloalkyl, and C₁-C₄ heteroalkyl.

51. The compound of claim 1, wherein R₇ is hydrogen.

52. The compound of claim 1, wherein R₈ is hydrogen.

53. (canceled)

54. The compound of claim 1, wherein R₉ is selected from among hydrogen, F, Cl, Br, CN, an optionally substituted C₁-C₄ alkyl, an optionally substituted C₁-C₄ haloalkyl, and an optionally substituted C₁-C₄ heteroalkyl.

55. The compound of claim 1, wherein R₉ is hydrogen.

56. The compound of claim 1, wherein R₇ and R₈ together form an optionally substituted 5-6 member ring and R₉ is selected from among hydrogen, F, Cl, Br, CN, an optionally substituted C₁-C₄ alkyl, an optionally substituted C₁-C₄ haloalkyl, an optionally substituted C₁-C₄ heteroalkyl.

57. The compound of claim 1, wherein R₈ and R₉ together form an optionally substituted 4-6 member ring and R₇ is selected from among hydrogen, F, Cl, Br, CN, an optionally substituted C₁-C₄ alkyl, an optionally substituted C₁-C₄ haloalkyl, an optionally substituted C₁-C₄ heteroalkyl, —CONR₁₄R₁₅, and an optionally substituted aryl.

58. The compound of claim 1, wherein R₁₀ is selected from among hydrogen, F, Cl, CH₃, and OCH₃.

59. The compound of claim 1, wherein R₁₀ is hydrogen.

60. The compound of claim 1, wherein R₁₁ is selected from among hydrogen, cyano, formyl, C₁-C₄alkyl, C₂-C₄alkenyl, C₂-C₄alkynyl, hydroxyC₁-C₄alkyl, haloC₁-C₄alkyl, haloC₂-C₄alkenyl, hydroxyC₁-C₄alkyl, hydroxyC₂-C₄alkenyl, cyanoC₁-C₄alkenyl, hydroxyC₂-C₄alkynyl, alkoxyalkoxyC₁-C₄alkyl, hydroxyhaloC₁-C₄alkyl, aminoC₁-C₄alkyl, C₁-C₄alkylaminoC₁-C₄alkyl, diC₁-C₄alkylaminoC₁-C₄alkyl, C₁-C₄alkylC₂-C₄alkenylaminoC₁-C₄alkyl, arylaminoC₁-C₄alkyl, C₂-C₄alkenylaminoC₁-C₄alkyl, cycloC₃-C₆alkylaminoC₁-C₄alkyl, hydroxyalkoxyalkyl, haloalkylcarbonyl, alkoxyalkoxyalkoxy, carboxyalkoxyalkyl, alkoxyhaloalkyl, alkoxycarbonylalkenyl, hydroxy C₁-C₄alkylcarbamoyl, N,N-diC₁-C₄alkylaminoC₁-C₄alkyl, N-cycloC₃-C₆alkyl-N—C₁-C₄alkylaminocarbonyl, haloC₁-C₄alkylcarbamoyl, hydroxyhaloC₁-C₄alkyl, C₁-C₄alkylcarbonyl, cycloC₃-C₆alkylcarbonyl, C₂-C₄alkenylcarbonyl, C₂-C₄alkynylcarbonyl, arylcarbonyl, heteroarylcarbonyl, hydroxyaralkyl, C₁-C₄alkoxyC₁-C₄alkyl, C₂-C₄alkenyloxyC₁-C₄alkyl, C₂-C₄alkynyloxyC₁-C₄alkyl, aryloxyC₁-C₄alkyl, hydroxyiminoC₁-C₄alkyl, alkoxyiminoC₁-C₄alkyl, C₂-C₄alkenyloxyiminoC₁-C₄alkyl, aryloxyiminoC₁-C₄alkyl, aralkoxyiminoC₁-C₄alkyl, heterocyclyl, heteroaryl and —CONR₁₄R₁₅, wherein the alkyl, alkenyl, alkynyl, cycloalkyl, heterocyclyl, heteroaryl and aryl groups can be unsubstituted or substituted with one to three substituents selected from among C₁-C₄alkyl, C₂-C₄alkenyl, C₂-C₄alkynyl, hydroxy, C₁-C₄alkoxy, nitro, halo, cyano, oxo, aryl, cycloalkyl, heterocyclyl, and heteroaryl groups.

61. The compound of claim 1, wherein R₁₁ is selected from among hydroxyC₁-C₄alkyl, hydroxyiminoC₁-C₄alkyl, C₁-C₄alkoxyiminoC₁-C₄alkyl, C₁-C₄alkylcarbonyl, C₁-C₄alkenyloxyiminoC₁-C₄alkyl, aryloxyiminoC₁-C₄alkyl, aralkoxyiminoC₁-C₄alkyl, C₁-C₄alkoxyC₁-C₄alkyl, C₁-C₄alkoxyalkoxyC₁-C₄alkyl, hydroxyhaloC₁-C₄alkyl, cycloalkylcarbonyl, C₂-C₄alkynylaminoC₁-C₄alkyl, haloC₁-C₄alkylaminoC₁-C₄alkyl, hydroxyalkoxyC₁-C₄alkyl, cyanoC₂-C₄alkenyl, alkoxyhaloC₁-C₄alkyl, heterocyclylcarbonyl and haloalkylcarbamoyl.

62. The compound of claim 1, wherein R₁₁ is selected from among hydrogen, cyano, carbamoyl, hydroxymethyl, 1-hydroxyethyl, vinyl, acetyl, 1-hydroxy-1-methylethyl, methoxymethyl, 4,4-fluorophenylhydroxy-methyl, cyclohexylhydroxymethyl, hydroxythien-3-ylmethyl, hydroxythien-2-ylmethyl, N,N-diethylaminocarbonyl, methoxymethoxymethyl, 3-prop-2-enyloxy-methyl, 1-hydroxybut-3-enyl, 1-hydroxy-2-phenylethyl, acroloyl, 4-fluorobenzoyl, thien-2-ylcarbonyl, cyclohexylcarbonyl, aminomethyl, phenylaminomethyl, prop-2-ynylaminomethyl, 2,2,2-trifluoroethylaminomethyl, cyclopropyl-aminomethyl, butylaminomethyl, 2-hydroxyethoxymethyl, isopropenyl, formyl, trifluoroacetyl, methoxyethoxymethoxy, 2,2,2-trifluoro-1-hydroxy-1-(trifluoromethyl)-ethyl, but-2-ynloxymethyl, 1-cyanovinyl, prop-3-ynyloxymethyl, 4-hydroxy-but-3-enyl, 1-hydroxy-2-trifluoroethyl, ethoxycarbonylmethoxymethyl, carboxy-methoxymethyl, 1-hydroxyprop-2-ynyl, 1-methoxy-2,2,2-trifluoroethyl, 2,2,2-trifluoro-1-methoxy-1-(trifluoromethyl)ethyl, 1-hydroxy-1-(thien-3-yl)ethyl, 2-methoxycarbonyl-vinyl, hydroxyethylcarbamoyl, ethylcarbamoyl, 2-(carbomethoxy)pyrrolidino-carbonyl, piperazinocarbonyl, N,N-dimethylaminomethyl, N,N-dimethylaminocarbonyl, N-ethyl-N-methylaminocarbonyl, N-morpholinocarbonyl, cyclopropyl, N-cyclohexyl-N-methylamino-carbonyl, 1-pyrrolidinocarbonyl, 2,2,2-trifluoroethylcarbamoyl, 4-hydroxypiperidinecarbonyl, 4-methylpiperazinecarbonyl, 1-hydroxy-4,4,4-trifluorobut-2-ynyl, 3-hydroxy-3-phenylpropanoyl, 3-hydroxy-3-butanoyl, N,N-dimethoxyethylaminocarbonyl, N-allyl-N-methylaminocarbonyl, 1-piperidinocarbonyl, 4-oxo-piperidi-1-nocarbonyl, 4-(1,3-dioxan)piperidinocarbonyl, piperidin-1-ylmethyl, benzoyl, 1-hydroxybenzyl, 1-hydroxyiminoethyl, 1-methoxy-iminoethyl, 1-allyloxy-iminoethyl, phenoxyiminoethyl, 1-ethoxyiminoethyl, 1-carboxymethoxyiminoethyl, 1-t-butyl-oxyiminoethyl, 1-benzyloxyiminoethyl, 1-(4-nitrobenzyl)oxyiminoethyl, 1-hydroxyiminomethyl, 1-hydroxyprop-2-ynyl, and but-2-enoyl.

63. The compound of claim 1, wherein R₁₁ is selected from among hydroxymethyl, acetyl, 1-hydroxy-1-methylethyl, 1-hydroxyethyl, 1-hydroxyiminoethyl, 1-methoxyiminoethyl, 1-allyloxyiminoethyl, 1-phenoxy-iminoethyl, 1-ethoxyiminoethyl, 1-tertbutoxyiminoethyl, 1-benzyloxy-iminoethyl, hydroxyiminomethyl, methoxymethyl, methoxymethoxymethyl, 1-hydroxy-2,2,2-trifluoroethyl, cyclohexylcarbonyl, prop-2-ynylaminomethyl, 2,2,2-trifluoroethyl-aminomethyl, 2-hydroxymethoxymethyl, 2-cyanovinyl, 1-methoxy-2,2,2-trifluoroethyl, 4-oxopiperidinocarbonyl, 2,2,2-trifluoroethylcarbamoyl, pyrrolidinocarbonyl and piperidinocarbonyl.

64. The compound of claim 1, wherein R₁₂ is selected from among hydrogen, F, Cl, Br, CN, an optionally substituted C₁-C₄ alkyl, an optionally substituted C₁-C₄ haloalkyl, an optionally substituted C₁-C₄ heteroalkyl, —OR₁₆, and an optionally substituted aryl.

65. The compound of claim 64, wherein R₁₂ is selected from among hydrogen, and an optionally substituted C₁-C₄ alkyl.

66. The compound of claim 64, wherein R₁₂ is hydrogen.

67. The compound of claim 60, wherein R₁₂ is C₁-C₄ alkyl.

68. The compound of claim 1, wherein R₁₃ is selected from among hydrogen, F, Cl, Br, CN, CONR₁₄R₁₅, an optionally substituted C₁-C₄ alkyl, an optionally substituted C₁-C₄ haloalkyl, and an optionally substituted C₁-C₄ heteroalkyl.

69. The compound of claim 68, wherein R₁₃ is selected from among hydrogen, optionally substituted C₁-C₄ alkyl and optionally substituted CONR₁₄R₁₅.

70. The compound of claim 1, wherein R₁₄ and R₁₅ are each independently hydrogen, C₁-C₄ alkyl or together with the nitrogen atom on which they are substituted form a heterocyclyl or heteroaryl ring that can be optionally substituted with one to three substituents selected from C₁-C₄ alkyl and halo.

71. The compound of claim 68, wherein R₁₃ is selected from among hydrogen, methyl, N,N-diethylaminocarbonyl, 1-pyrrolidinocarbonyl, 2-methylpyrrolidi-1-nocarbonyl, and 1-morpholinocarbonyl.

72. The compound of claim 1, wherein R₁₁ and R₁₂ together form an optionally substituted 5-6 member ring and R₁₃ is selected from among hydrogen, F, Cl, Br, CN, CONR₁₄R₁₅, an optionally substituted C₁-C₄ alkyl, an optionally substituted C₁-C₄ haloalkyl, and an optionally substituted C₁-C₄ heteroalkyl.

73. The compound of claim 1, wherein R₁₂ and R₁₃ together form an optionally substituted 4-6 member ring and R₁₁, is selected from among hydrogen, F, Cl, Br, CN, an optionally substituted C₁-C₄ alkyl, an optionally substituted C₁-C₄ haloalkyl, an optionally substituted C₁-C₄ heteroalkyl, —CONR₁₄R₁₅, and an optionally substituted aryl.

74. The compound of claim 1, wherein R₁₄ and R₁₅ are each independently selected from among hydrogen, an optionally substituted C₁-C₄ alkyl, an optionally substituted C₁-C₄ haloalkyl, and an optionally substituted C₁-C₄ heteroalkyl.

75. The compound of claim 1, wherein R₁₄ and R₁₅ together form an optionally substituted 4-7 member ring.

76. The compound of claim 1, wherein R₁₆ is selected from among hydrogen, an optionally substituted C₁-C₄ alkyl, an optionally substituted C₁-C₄ haloalkyl, an optionally substituted C₁-C₄ heteroalkyl, and an optionally substituted aryl.

77. The compound of claim 1, wherein R₁₆ is an optionally substituted phenyl.

78. The compound of claim 1, wherein X is selected from among O, S, and NR₁₇.

79. The compound of claim 78, wherein X is O.

80. The compound of claim 78, wherein X is S.

81. The compound of claim 78, wherein X is NR₁₇.

82. The compound of claim 81, wherein R₁₇ is selected from among hydrogen and an optionally substituted C₁-C₄ alkyl.

83. The compound of claim 81, wherein R₁₇ is hydrogen.

84. The compound of claim 1, wherein R₁ is 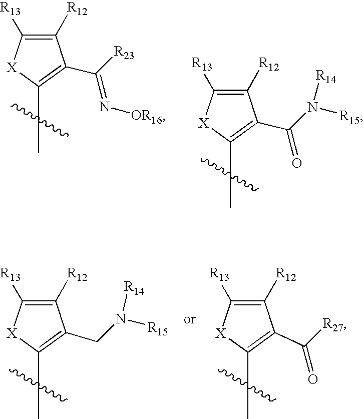
wherein R₂₃ is selected from among hydrogen, optionally substituted C₁-C₄ alkyl, optionally substituted C₁-C₄ alkenyl, optionally substituted C₁-C₄ alkynyl and optionally substituted aryl; and
R₂₇ is selected from among hydrogen, optionally substituted C₁-C₄ alkyl, optionally substituted C₁-C₄ alkenyl, optionally substituted C₁-C₄ alkynyl, optionally substituted aryl, optionally substituted heteroaryl, optionally substituted cycloalkyl, optionally substituted heterocyclyl.

85. (canceled)

86. The compound of claim 84, wherein R₁ is

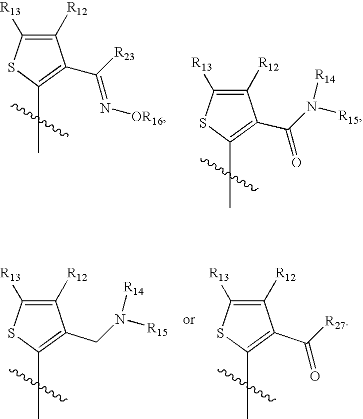

87. The compound of claim 1, wherein R₁ is

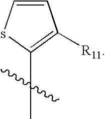

88. The compound of claim 87, wherein R₁₁ is selected from among hydroxyC₁-C₄alkyl, hydroxyiminoC₁-C₄alkyl, C₁-C₄alkoxyiminoC₁-C₄alkyl, C₁-C₄alkylcarbonyl, C₁-C₄alkenyloxyiminoC₁-C₄alkyl, aryloxyiminoC₁-C₄alkyl, aralkoxyimino-C₁-C₄alkyl, C₁-C₄alkoxyC₁-C₄alkyl, C₁-C₄alkoxyalkoxyC₁-C₄alkyl, hydroxyhaloC₁-C₄alkyl, cycloalkylcarbonyl, C₂-C₄alkynylaminoC₁-C₄alkyl, haloC₁-C₄alkylaminoC₁-C₄alkyl, hydroxyalkoxyC₁-C₄alkyl, cyanoC₂-C₄alkenyl, alkoxyhaloC₁-C₄alkyl, heterocyclylcarbonyl and haloalkylcarbamoyl.

89. The compound of claim 84, wherein R₁ is

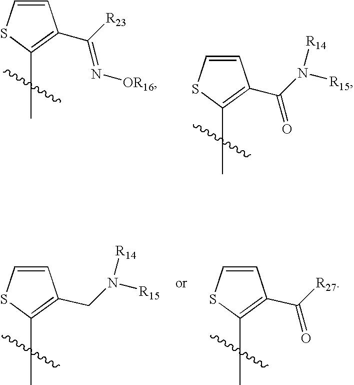

90. The compound of claim 76, wherein R₁ is

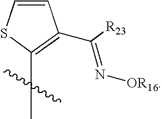

91. The compound of claim 90, wherein R₁₆ is hydrogen, methyl, allyl, tert-butyl, and benzyl.

92. The compound of claim 90, wherein R₂₃ is hydrogen or methyl.

93. The compound of claim 89, wherein R₁ is

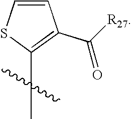

94. The compound of claim 93, wherein R₂₇ is methyl, cyclohexyl, 4-oxo-piperidinyl, pyrrolidinyl, or piperidinyl.

95. The compound of claim 89, wherein R₁ is

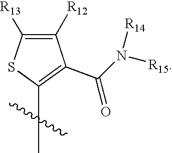

96. The compound of claim 1, wherein R₁₄ and R₁₅ are each independently hydrogen, alkyl, haloalkyl or aryl, or R₁₄ and R₁₅ together with the nitrogen atom on which they are substituted form an optionally substituted heterocyclyl or optionally substituted heteroaryl ring.

97. The compound of claim 96, wherein R₁₄ and R₁₅ are each independently hydrogen, methyl, trifluoroethyl, or R₁₄ and R₁₅ together with the nitrogen atom on which they are substituted form a pyrrolidinyl, 4-oxopiperidinyl or piperidinyl ring.

98. The compound of claim 89, wherein R₁ is

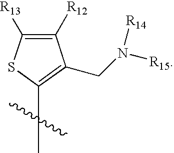

99. The compound of claim 1, wherein the compound has Formula V:

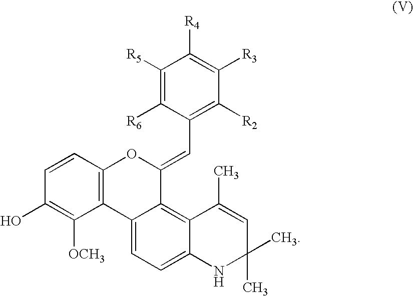

100. The compound of claim 1, wherein the compound has Formula VI or VII:

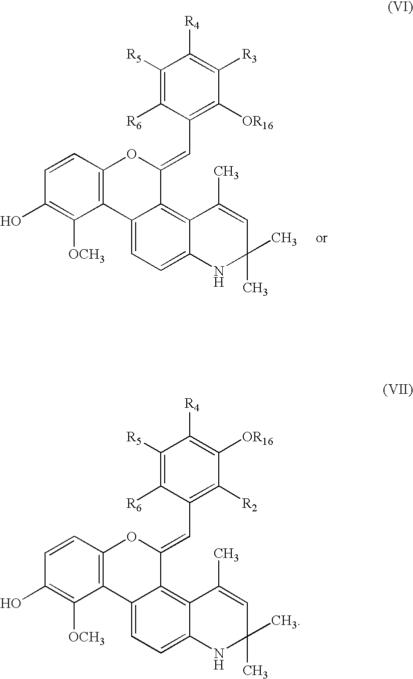

101. The compound of claim 1, wherein the compound has Formula VIII:

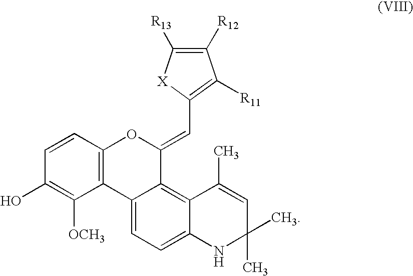

102. The compound of claim 1, wherein the compound has Formula IX:

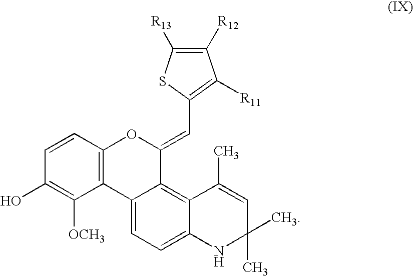

103. The compound of claim 1, wherein the compounds has Formula (X):

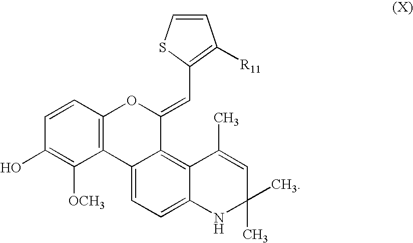

104. The compound of claim 1, wherein the compound has Formula XI:

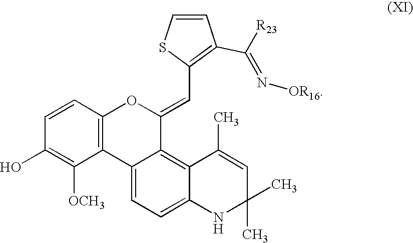

105. The compound of claim 1, wherein the compound has Formula XII:

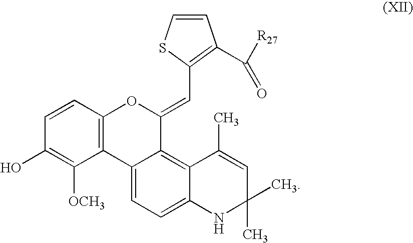

106. The compound of claim 1, wherein the compound has Formula XIII:

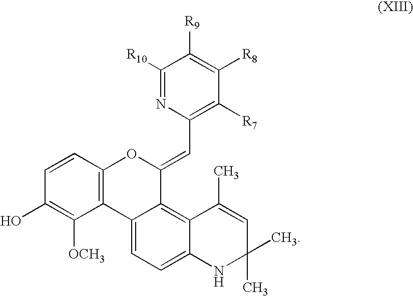

107. The compound of claim 1 selected from among:
(Z)-5-(3′-trifluoromethylbenzylidene)-1,2-dihydro-9-hydroxy-10-methoxy-2,2,4-trimethyl-5H-chromeno[3,4-f]quinoline (compound 11);
(Z)-5-(2′-fluorobenzylidene)-1,2-dihydro-9-hydroxy-10-methoxy-2,2,4-trimethyl-5H-chromeno[3,4-f]quinoline (compound 12);
(Z)-5-(3′-chlorobenzylidene)-1,2-dihydro-9-hydroxy-10-methoxy-2,2,4-trimethyl-5H-chromeno[3,4-f]quinoline (compound 13);
(Z)-5-(2′,5′-dichlorobenzylidene)-1,2-dihydro-9-hydroxy-10-methoxy-2,2,4-trimethyl-5H-chromeno[3,4-f]quinoline (compound 14);
(Z)-5-(3′-methoxybenzylidene)-1,2-dihydro-9-hydroxy-10-methoxy-2,2,4-trimethyl-5H-chromeno[3,4-f]quinoline (compound 15);
(Z)-5-(2′-chlorobenzylidene)-1,2-dihydro-9-hydroxy-10-methoxy-2,2,4-trimethyl-5H-chromeno[3,4-f]quinoline (compound 16);
(Z)-5-(4′-chlorobenzylidene)-1,2-dihydro-9-hydroxy-10-methoxy-2,2,4-trimethyl-5H-chromeno[3,4-f]quinoline (compound 17);
(Z)-5-(3′-methylbenzylidene)-1,2-dihydro-9-hydroxy-10-methoxy-2,2,4-trimethyl-5H-chromeno[3,4-f]quinoline (compound 18);
(Z)-5-(4′-methylbenzylidene)-1,2-dihydro-9-hydroxy-10-methoxy-2,2,4-trimethyl-5H-chromeno[3,4-f]quinoline (compound 19);
(Z)-5-(4′-methoxybenzylidene)-1,2-dihydro-9-hydroxy-10-methoxy-2,2,4-trimethyl-5H-chromeno[3,4-f]quinoline (compound 20);
(Z)-5-(2′-bromobenzylidene)-1,2-dihydro-9-hydroxy-10-methoxy-2,2,4-trimethyl-5H-chromeno[3,4-f]quinoline (compound 21);
(Z)-5-(3′-trifluoromethoxybenzylidene)-1,2-dihydro-9-hydroxy-10-methoxy-2,2,4-trimethyl-5H-chromeno[3,4-f]quinoline (compound 22);
(Z)-5-(3′,5′-dichlorobenzylidene)-1,2-dihydro-9-hydroxy-10-methoxy-2,2,4-trimethyl-5H-chromeno[3,4-f]quinoline (compound 23);
(Z)-5-(3′-bromobenzylidene)-1,2-dihydro-9-hydroxy-10-methoxy-2,2,4-trimethyl-5H-chromeno[3,4-f]quinoline (compound 24);
(Z)-5-(2′-chloro-4′-fluorobenzylidene)-1,2-dihydro-9-hydroxy-10-methoxy-2,2,4-trimethyl-5H-chromeno[3,4-f]quinoline (compound 25);
(Z)-5-(4′-trifluoromethoxybenzylidene)-1,2-dihydro-9-hydroxy-10-methoxy-2,2,4-trimethyl-5H-chromeno[3,4-f]quinoline (compound 26);
(Z)-5-(3′-trifluoromethylthiobenzylidene)-1,2-dihydro-9-hydroxy-10-methoxy-2,2,4-trimethyl-5H-chromeno[3,4-f]quinoline (compound 27);
(Z)-5-(2′-fluoro-3′-methylbenzylidene)-1,2-dihydro-9-hydroxy-10-methoxy-2,2,4-trimethyl-5H-chromeno[3,4-f]quinoline (compound 28);
(Z)-5-(2′-fluoro-3′-trifluoromethylbenzylidene)-1,2-dihydro-9-hydroxy-10-methoxy-2,2,4-trimethyl-5H-chromeno[3,4-f]quinoline (compound 29);
(Z)-5-(3′,4′-dichlorobenzylidene)-1,2-dihydro-9-hydroxy-10-methoxy-2,2,4-trimethyl-5H-chromeno[3,4-f]quinoline (compound 30);
(Z)-5-(4′-chloro-3′-trifluoromethylbenzylidene)-1,2-dihydro-9-hydroxy-10-methoxy-2,2,4-trimethyl-5H-chromeno[3,4-f]quinoline (compound 31);
(Z)-5-(3′,5′-di(trifluoromethyl)benzylidene)-1,2-dihydro-9-hydroxy-10-methoxy-2,2,4-trimethyl-5H-chromeno[3,4-f]quinoline (compound 32);
(Z)-5-(3′-fluoro-5′-trifluoromethylbenzylidene)-1,2-dihydro-9-hydroxy-10-methoxy-2,2,4-trimethyl-5H-chromeno[3,4-f]quinoline (compound 33);
(Z)-5-(2′,4′,5′-trifluorobenzylidene)-1,2-dihydro-9-hydroxy-10-methoxy-2,2,4-trimethyl-5H-chromeno[3,4-f]quinoline (compound 34);
(Z)-5-(2′-methylbenzylidene)-1,2-dihydro-9-hydroxy-10-methoxy-2,2,4-trimethyl-5H-chromeno[3,4-f]quinoline (compound 35);
(Z)-5-(4′-ethylbenzylidene)-1,2-dihydro-9-hydroxy-10-methoxy-2,2,4-trimethyl-5H-chromeno[3,4-f]quinoline (compound [36]);
(Z)-5-(5′-fluoro-2′-methylbenzylidene)-1,2-dihydro-9-hydroxy-10-methoxy-2,2,4-trimethyl-5H-chromeno[3,4-f]quinoline (compound 37);
(Z)-5-(2′-chloro-6′-fluorobenzylidene)1,2-dihydro-9-hydroxy-10-methoxy-2,2,4-trimethyl-5H-chromeno[3,4-f]quinoline (compound [38]);
(Z)-5-(4′-isopropylbenzylidene)-1,2-dihydro-9-hydroxy-10-methoxy-2,2,4-trimethyl-5H-chromeno[3,4-f]quinoline (compound 39);
(Z)-5-(4′-bromobenzylidene)-1,2-dihydro-9-hydroxy-10-methoxy-2,2,4-trimethyl-5H-chromeno[3,4-f]quinoline (compound 40);
(Z)-5-(3′-fluoro-4′-methylbenzylidene)-1,2-dihydro-9-hydroxy-10-methoxy-2,2,4-trimethyl-5H-chromeno[3,4-f]quinoline (compound 41);
(Z)-5-(2′-(6′-methyl-pyridinylmethylidene))-1,2-dihydro-9-hydroxy-10-methoxy-2,2,4-trimethyl-5H-chromeno[3,4-f]quinoline (compound 42);
(Z)-5-(2′-methyl-3′-trifluoromethylbenzylidene)-1,2-dihydro-9-hydroxy-10-methoxy-2,2,4-trimethyl-5H-chromeno[3,4-f]quinoline (compound 43);
(Z)-5-(4′-benzyloxybenzylidene)-1,2-dihydro-9-hydroxy-10-methoxy-2,2,4-trimethyl-5H-chromeno[3,4-f]quinoline (compound 44);
(Z)-5-(2′-phenylbenzylidene)-1,2-dihydro-9-hydroxy-10-methoxy-2,2,4-trimethyl-5H-chromeno[3,4-f]quinoline (compound 46);
(Z)-5-(4′-phenylbenzylidene)-1,2-dihydro-9-hydroxy-10-methoxy-2,2,4-trimethyl-5H-chromeno[3,4-f]quinoline (compound 47);
(Z)-5-(3′-methyl-4′-fluorobenzylidene)-1,2-dihydro-9-hydroxy-10-methoxy-2,2,4-trimethyl-5H-chromeno[3,4-f]quinoline (compound 48);
(Z)-5-(4′-cyclohexylbenzylidene)-1,2-dihydro-9-hydroxy-10-methoxy-2,2,4-trimethyl-5H-chromeno[3,4-f]quinoline (compound 49);
(Z)-5-(2′-chloro-3′-trifluoromethylbenzylidene)-1,2-dihydro-9-hydroxy-10-methoxy-2,2,4-trimethyl-5H-chromeno[3,4-f]quinoline (compound 51);
(Z)-5-(3′-phenylbenzylidene)-1,2-dihydro-9-hydroxy-10-methoxy-2,2,4-trimethyl-5H-chromeno[3,4-f]quinoline (compound 52);
(Z)-5-(3′-chloro-4′-trifluoromethoxybenzylidene)-1,2-dihydro-9-hydroxy-10-methoxy-2,2,4-trimethyl-5H-chromeno[3,4-f]quinoline (compound 54);
(Z)-5-(2′,6′-difluoro-3′-chlorobenzylidene)-1,2-dihydro-9-hydroxy-10-methoxy-2,2,4-trimethyl-5H-chromeno[3,4-f]quinoline (compound 55);
(Z)-5-(2′-chloro-3′,6′-difluorobenzylidene)-1,2-dihydro-9-hydroxy-10-methoxy-2,2,4-trimethyl-5H-chromeno[3,4-f]quinoline (compound 56);
(Z)-5-(4′-methyl-3′-trifluoromethylbenzylidene)-1,2-dihydro-9-hydroxy-10-methoxy-2,2,4-trimethyl-5H-chromeno[3,4-f]quinoline (compound 58);
(Z)-5-(2′-fluoro-4′-chlorobenzylidene)-1,2-dihydro-9-hydroxy-10-methoxy-2,2,4-trimethyl-5H-chromeno[3,4-f]quinoline (compound 59);
(Z)-5-(2′,3′-difluoro-4′-methylbenzylidene)-1,2-dihydro-9-hydroxy-10-methoxy-2,2,4-trimethyl-5H-chromeno[3,4-f]quinoline (compound 60);
(Z)-5-(2′,3′,5′,6′-tetrafluoro-4′-trifluoromethylbenzylidene)-1,2-dihydro-9-hydroxy-10-methoxy-2,2,4-trimethyl-5H-chromeno[3,4-f]quinoline (compound 61);
(Z)-5-(2′-(3′-(dimethylaminocarbonyl)furanylmethylidene))-1,2-dihydro-9-hydroxy-10-methoxy-2,2,4-trimethyl-5H-chromeno[3,4-f]quinoline (compound 62);
(Z)-5-(4′-vinylbenzylidene)-1,2-dihydro-9-hydroxy-10-methoxy-2,2,4-trimethyl-5H-chromeno[3,4-f]quinoline (compound 63);
(Z)-5-(2′-Chloro-6′-fluoro-5′-methylbenzylidene)-1,2-dihydro-9-hydroxy-10-methoxy-2,2,4-trimethyl-5H-chromeno[3,4-f]quinoline (compound 64);
(Z)-5-(2′-trifluoromethoxybenzylidene)-1,2-dihydro-9-hydroxy-10-methoxy-2,2,4-trimethyl-5H-chromeno[3,4-f]quinoline (compound 65);
(Z)-5-(2′-trifluoromethylthiobenzylidene)-1,2-dihydro-9-hydroxy-10-methoxy-2,2,4-trimethyl-5H-chromeno[3,4-f]quinoline (compound 66);
(Z)-5-(3′,4′-methylenedioxybenzylidene)-1,2-dihydro-9-hydroxy-10-methoxy-2,2,4-trimethyl-5H-chromeno[3,4-f]-quinoline (compound 67);
(Z)-5-(3′-chloro-2′-fluorobenzylidene)-1,2-dihydro-9-hydroxy-10-methoxy-2,2,4-trimethyl-5H-chromeno[3,4-f]quinoline (compound 68);
(Z)-5-(4′-(4″-methylbenzyloxy)benzylidene)-1,2-dihydro-9-hydroxy-10-methoxy-2,2,4-trimethyl-5H-chromeno[3,4-f]quinoline (compound 70);
(Z)-5-(3′,5′-di-tert-butylbenzylidene)-1,2-dihydro-9-hydroxy-10-methoxy-2,2,4-trimethyl-5H-chromeno[3,4-f]quinoline (compound 71);
(Z)-5-(3′-(2″,2″-difluoroethoxy)benzylidene)-1,2-dihydro-9-hydroxy-10-methoxy-2,2,4-trimethyl-5H-chromeno[3,4-f]quinoline (compound 72);
(Z)-5-(2′,5′-dimethylbenzylidene)-1,2-dihydro-9-hydroxy-10-methoxy-2,2,4-trimethyl-5H-chromeno[3,4-f]quinoline (compound 73);
(Z)-5-(3′-(3″-thienyl)benzylidene)-1,2-dihydro-9-hydroxy-10-methoxy-2,2,4-trimethyl-5H-chromeno[3,4-f]quinoline (compound 74);
(Z)-5-(2′-diethylaminocarbonylbenzylidene)-1,2-dihydro-9-hydroxy-10-methoxy-2,2,4-trimethyl-5H-chromeno[3,4-f]quinoline (compound 75);
(Z)-5-(3′-(4″,4″,4″-trifluorobutoxy)benzylidene)-1,2-dihydro-9-hydroxy-10-methoxy-2,2,4-trimethyl-5H-chromeno[3,4-f]quinoline (compound 76);
(Z)-5-(3′-(2″,4″-difluorophenyl)benzylidene)-1,2-dihydro-9-hydroxy-10-methoxy-2,2,4-trimethyl-5H-chromeno[3,4-f]quinoline (compound 77);
(Z)-5-(3′-(3″-pyridyl)benzylidene)-1,2-dihydro-9-hydroxy-10-methoxy-2,2,4-trimethyl-5H-chromeno[3,4-f]quinoline (compound 78);
(Z)-5-(2′-(3″-formylphenyl)benzylidene)-1,2-dihydro-9-hydroxy-10-methoxy-2,2,4-trimethyl-5H-chromeno[3,4-f]quinoline (compound 79);
(Z)-5-(3′,5′-dimethylbenzylidene)-1,2-dihydro-9-hydroxy-10-methoxy-2,2,4-trimethyl-5H-chromeno[3,4-f]quinoline (compound 80);
(Z)-5-(3′,4′-dimethylbenzylidene)-1,2-dihydro-9-hydroxy-10-methoxy-2,2,4-trimethyl-5H-chromeno[3,4-f]quinoline (compound 81);
(Z)-5-(2′-(diethylamino)carbonyl-6′-fluorobenzylidene)-1,2-dihydro-9-hydroxy-10-methoxy-2,2,4-trimethyl-5H-chromeno[3,4-f]quinoline (compound 82);
(Z)-5-(2′-(diethylamino)carbonyl-4′-fluorobenzylidene)-1,2-dihydro-9-hydroxy-10-methoxy-2,2,4-trimethyl-5H-chromeno[3,4-f]quinoline (compound 83);
(Z)-5-(2′-(methylbenzylamino)carbonyl-6′-fluorobenzylidene)-1,2-dihydro-9-hydroxy-10-methoxy-2,2,4-trimethyl-5H-chromeno[3,4-f]quinoline (compound 84);
(Z)-5-(2′-(dimethylamino)carbonyl-5′-bromo-benzylidene)-1,2-dihydro-9-hydroxy-10-methoxy-2,2,4-trimethyl-5H-chromeno[3,4-f]quinoline (compound 85);
(Z)-5-(3′-(2″-fluoroethoxy)benzylidene)-1,2-dihydro-9-hydroxy-10-methoxy-2,2,4-trimethyl-5H-chromeno[3,4-f]quinoline (compound 86);
(Z)-5-(3′-(2″,2″,3″,3″-tetrafluoropropoxy)benzylidene)-1,2-dihydro-9-hydroxy-10-methoxy-2,2,4-trimethyl-5H-chromeno[3,4-f]quinoline (compound 87);
(Z)-5-(3′-(4″-fluorobenzyloxy)benzylidene)-1,2-dihydro-9-hydroxy-10-methoxy-2,2,4-trimethyl-5H-chromeno[3,4-f]quinoline (compound 88);
(Z)-5-(3′-(2″-fluorobenzyloxy)benzylidene)-1,2-dihydro-9-hydroxy-10-methoxy-2,2,4-trimethyl-5H-chromeno[3,4-f]quinoline (compound 89);
(Z)-5-(2′-(pyrrolidinecarbonyl)benzylidene)-1,2-dihydro-9-hydroxy-10-methoxy-2,2,4-trimethyl-5H-chromeno[3,4-f]quinoline (compound 90);
(Z)-5-(2′-(pyrrolidinecarbonyl)-5′-bromobenzylidene)-1,2-dihydro-9-hydroxy-10-methoxy-2,2,4-trimethyl-5H-chromeno[3,4-f]quinoline (compound 91);
(Z)-5-(2′-(dimethylaminocarbonyl)-4′-fluorobenzylidene)-1,2-dihydro-9-hydroxy-10-methoxy-2,2,4-trimethyl-5H-chromeno[3,4-f]quinoline (compound 92);
(Z)-5-(2′-(pyrrolidinecarbonyl)-5′-methylbenzylidene)-1,2-dihydro-9-hydroxy-10-methoxy-2,2,4-trimethyl-5H-chromeno[3,4-f]quinoline (compound 93);
(Z)-5-(2′-(pyrrolidinecarbonyl)-4′-fluorobenzylidene)-1,2-dihydro-9-hydroxy-10-methoxy-2,2,4-trimethyl-5H-chromeno[3,4-f]quinoline (compound 94);
(Z)-5-(3′-(4″-fluorophenoxy)benzylidene)-1,2-dihydro-9-hydroxy-10-methoxy-2,2,4-trimethyl-5H-chromeno[3,4-f]quinoline (compound 95);
(Z)-5-(2′-(morpholinecarbonyl)-4′-fluorobenzylidene)-1,2-dihydro-9-hydroxy-10-methoxy-2,2,4-trimethyl-5H-chromeno[3,4-f]quinoline (compound 96);
(Z)-5-(8′-(6′-fluoro-benzo-1′,3′-dioxan-methylidene))-1,2-dihydro-9-hydroxy-10-methoxy-2,2,4-trimethyl-5H-chromeno[3,4-f]quinoline (compound 97);
(Z)-5-(2′-dimethylaminocarbonyl-3′-methoxybenzylidene)-1,2-dihydro-9-hydroxy-10-methoxy-2,2,4-trimethyl-5H-chromeno[3,4-f]quinoline (compound 98);
(Z)-5-(2′-(4″-methylpiperazinecarbonyl)-4′-fluorobenzylidene)-1,2-dihydro-9-hydroxy-10-methoxy-2,2,4-trimethyl-5H-chromeno[3,4-f]quinoline (compound 99);
(Z)-5-(2′-methyl-3′-phenylbenzylidene)-1,2-dihydro-9-hydroxy-10-methoxy-2,2,4-trimethyl-5H-chromeno[3,4-f]quinoline (compound 100);
(Z)-5-(3′,5′-di-methoxybenzylidene)-1,2-dihydro-9-hydroxy-10-methoxy-2,2,4-trimethyl-5H-chromeno[3,4-f]quinoline (compound 101);
(Z)-5-(2′-(piperidinecarbonyl)-4′-fluorobenzylidene)-1,2-dihydro-9-hydroxy-10-methoxy-2,2,4-trimethyl-5H-chromeno[3,4-f]quinoline (compound 102);
(Z)-5-(2′-dimethylaminosulphonyl-4′-fluorobenzylidene)-1,2-dihydro-9-hydroxy-10-methoxy-2,2,4-trimethyl-5H-chromeno[3,4-f]quinoline (compound 103);
(Z)-5-(3′-phenoxybenzylidene)-1,2-dihydro-9-hydroxy-10-methoxy-2,2,4-trimethyl-5H-chromeno[3,4-f]quinoline (compound 104);
(Z)-5-(2′-(ethylmethylamino)carbonyl-4′-fluorobenzylidene)-1,2-dihydro-9-hydroxy-10-methoxy-2,2,4-trimethyl-5H-chromeno[3,4-f]quinoline (compound 105);
(Z)-5-(2′-(cyclohexylmethylamino)carbonyl-4′-fluorobenzylidene)-1,2-dihydro-9-hydroxy-10-methoxy-2,2,4-trimethyl-5H-chromeno[3,4-f]quinoline (compound 106);
(Z)-5-(2′-cyanobenzylidene)-1,2-dihydro-9-hydroxy-10-methoxy-2,2,4-trimethyl-5H-chromeno[3,4-f]quinoline (compound 107);
(Z)-5-(2′,3′,5′,6′-tetrafluoro-4′-methoxybenzylidene)-1,2-dihydro-9-hydroxy-10-methoxy-2,2,4-trimethyl-5H-chromeno[3,4-f]quinoline (compound 108);
(Z)-5-(3′-hydroxybenzylidene)-1,2-dihydro-9-hydroxy-10-methoxy-2,2,4-trimethyl-5H-chromeno[3,4-f]quinoline (compound 109);
(Z)-5-(2′-(piperidinesulphonyl)-4′-fluorobenzylidene)-1,2-dihydro-9-hydroxy-10-methoxy-2,2,4-trimethyl-5H-chromeno[3,4-f]quinoline (compound 110);
(Z)-5-(1′-naphthylmethylidene)-1,2-dihydro-9-hydroxy-10-methoxy-2,2,4-trimethyl-5H-chromeno[3,4-f]quinoline (compound 111);
(Z)-5-(3′-methyl-4′-methoxybenzylidene)-1,2-dihydro-9-hydroxy-10-methoxy-2,2,4-trimethyl-5H-chromeno[3,4-f]quinoline (compound 112);
(Z)-5-(2′,5′-dimethoxybenzylidene)-1,2-dihydro-9-hydroxy-10-methoxy-2,2,4-trimethyl-5H-chromeno[3,4-f]quinoline (compound 113);
(Z)-5-(2′,3′-methylenedioxybenzylidene)-1,2-dihydro-9-hydroxy-10-methoxy-2,2,4-trimethyl-5H-chromeno[3,4-f]quinoline (compound 114);
(Z)-5-(2′,3′-ethylenedioxybenzylidene)-1,2-dihydro-9-hydroxy-10-methoxy-2,2,4-trimethyl-5H-chromeno[3,4-f]quinoline (compound 115);
(Z)-5-(4′-hydroxybenzylidene)-1,2-dihydro-9-hydroxy-10-methoxy-2,2,4-trimethyl-5H-chromeno[3,4-f]quinoline (compound 116);
(Z)-5-(2′-cyano-3′-methylbenzylidene)-1,2-dihydro-9-hydroxy-10-methoxy-2,2,4-trimethyl-5H-chromeno[3,4-f]quinoline (compound 117);
(Z)-5-(3′-chloro-2′-cyanobenzylidene)-1,2-dihydro-9-hydroxy-10-methoxy-2,2,4-trimethyl-5H-chromeno[3,4-f]quinoline (compound 118);
(Z)-5-(5′-bromo-2′-cyano-benzylidene)-1,2-dihydro-9-hydroxy-10-methoxy-2,2,4-trimethyl-5H-chromeno[3,4-f]quinoline (compound 119);
(Z)-5-(8′-(6′-chloro-benzo-1′,3′-dioxan-methylidene)-1,2-dihydro-9-hydroxy-10-methoxy-2,2,4-trimethyl-5H-chromeno[3,4-f]quinoline (compound 120);
(Z)-5-(2′-chloro-3′,4′-dimethoxybenzylidene)-1,2-dihydro-9-hydroxy-10-methoxy-2,2,4-trimethyl-5H-chromeno[3,4-f]quinoline (compound 121);
(Z)-5-(2′-cyano-3′-fluorobenzylidene)-1,2-dihydro-9-hydroxy-10-methoxy-2,2,4-trimethyl-5H-chromeno[3,4-f]quinoline (compound 122);
(Z)-5-(8′-(6′-methyl-benzo-1′,3′-dioxan-methylidene))-1,2-dihydro-9-hydroxy-10-methoxy-2,2,4-trimethyl-5H-chromeno[3,4-f]quinoline (compound 123);
(Z)-5-(2′-cyano-5′-methylbenzylidene)-1,2-dihydro-9-hydroxy-10-methoxy-2,2,4-trimethyl-5H-chromeno[3,4-f]quinoline (compound 124);
(Z)-5-(8′-(5′,6′-difluoro-benzo-1′,3′-dioxan-methylidene))-1,2-dihydro-9-hydroxy-10-methoxy-2,2,4-trimethyl-5H-chromeno[3,4-f]quinoline (compound 125);
(Z)-5-(3′-(3″,5″-dichlorophenoxy)benzylidene)-1,2-dihydro-9-hydroxy-10-methoxy-2,2,4-trimethyl-5H-chromeno[3,4-f]quinoline (compound 126);
(Z)-5-(3′-(4″-methoxyphenoxy)benzylidene)-1,2-dihydro-9-hydroxy-10-methoxy-2,2,4-trimethyl-5H-chromeno[3,4-f]quinoline (compound 127);
(Z)-5-(3′-(3″,4″-dichlorophenoxy)benzylidene)-1,2-dihydro-9-hydroxy-10-methoxy-2,2,4-trimethyl-5H-chromeno[3,4-f]quinoline (compound 128);
(Z)-5-(3′-(4″-methylphenoxy)benzylidene)-1,2-dihydro-9-hydroxy-10-methoxy-2,2,4-trimethyl-5H-chromeno[3,4-f]quinoline (compound 129);
(Z)-5-(3′-(4″-chlorophenoxy)benzylidene)-1,2-dihydro-9-hydroxy-10-methoxy-2,2,4-trimethyl-5H-chromeno[3,4-f]quinoline (compound 130);
(Z)-5-(3′-(3″-trifluoromethoxyphenoxy)benzylidene)-1,2-dihydro-9-hydroxy-10-methoxy-2,2,4-trimethyl-5H-chromeno[3,4-f]quinoline (compound 131);
(Z)-5-(2′-(3′-(dimethylaminocarbonyl)thienylmethylidene))-1,2-dihydro-9-hydroxy-10-methoxy-2,2,4-trimethyl-5H-chromeno[3,4-f]quinoline (compound 132);
(Z)-5-(2′-(3′-(ethylmethylaminocarbonyl)thienylmethylidene))-1,2-dihydro-9-hydroxy-10-methoxy-2,2,4-trimethyl-5H-chromeno[3,4-f]quinoline (compound 134);
(Z)-5-(2′-(3′-(morpholinocarbonyl)thienylmethylidene)-1,2-dihydro-9-hydroxy-10-methoxy-2,2,4-trimethyl-5H-chromeno[3,4-f]quinoline (compound 135);
(Z)-5-(2′-(3′-(cyclohexylmethylaminocarbonyl)thienylmethylidene))-1,2-dihydro-9-hydroxy-10-methoxy-2,2,4-trimethyl-5H-chromeno[3,4-f]quinoline (compound 136);
(Z)-5-(2′-(3′-(pyrrolidinocarbonyl)thienylmethylidene))-1,2-dihydro-9-hydroxy-10-methoxy-2,2,4-trimethyl-5H-chromeno[3,4-f]quinoline (compound 137);
(Z)-5-(2′-(3′-(di(methoxyethyl)aminocarbonyl)thienylmethylidene)-1,2-dihydro-9-hydroxy-10-methoxy-2,2,4-trimethyl-5H-chromeno[3,4-f]quinoline (compound 138);
(Z)-5-(2′-(3′-(allylmethylaminocarbonyl)thienylmethylidene))-1,2-dihydro-9-hydroxy-10-methoxy-2,2,4-trimethyl-5H-chromeno[3,4-f]quinoline (compound 139);
(Z)-5-(2′-(3′-(piperidinocarbonyl)thienylmethylidene))-1,2-dihydro-9-hydroxy-10-methoxy-2,2,4-trimethyl-5H-chromeno[3,4-f]quinoline (compound 140);
(Z)-5-(2′-(3′-piperidinecarbonyl-4″-(1,3-dioxan)thienylmethylidene)-1,2-dihydro-9-hydroxy-10-methoxy-2,2,4-trimethyl-5H-chromeno[3,4-f]quinoline (compound 141);
(Z)-5-(2′-(5′-(diethylaminocarbonyl)thienylmethylidene))-1,2-dihydro-9-hydroxy-10-methoxy-2,2,4-trimethyl-5H-chromeno[3,4-f]quinoline (compound 142);
(Z)-5-(2′-(5′-(pyrrolidinocarbonyl)thienylmethylidene))-1,2-dihydro-9-hydroxy-10-methoxy-2,2,4-trimethyl-5H-chromeno[3,4-f]quinoline (compound 143);
(Z)-5-(2′-(5′-(2″-methylpyrrolidinocarbonyl)thienylmethylidene))-1,2-dihydro-9-hydroxy-10-methoxy-2,2,4-trimethyl-5H-chromeno[3,4-f]quinoline (compound 144);
(Z)-5-(2′-(5′-(morpholinocarbonyl)thienylmethylidene))-1,2-dihydro-9-hydroxy-10-methoxy-2,2,4-trimethyl-5H-chromeno[3,4-f]quinoline (compound 145);
(Z)-5-(2′-(3′-dimethylaminocarbonyl-5′-methylfuranylmethylidene))-1,2-dihydro-9-hydroxy-10-methoxy-2,2,4-trimethyl-5H-chromeno[3,4-f]quinoline (compound 146);
(Z)-5-(2′-(3′-cyclohexylmethylaminocarbonyl-5′-methylfuranyl-methylidene))-1,2-dihydro-9-hydroxy-10-methoxy-2,2,4-trimethyl-5H-chromeno[3,4-f]quinoline (compound 147);
(Z)-5-(4′-(2″-fluorophenyl)benzylidene)1,2-dihydro-9-hydroxy-10-methoxy-2,2,4-trimethyl-5H-chromeno[3,4-f]quinoline (compound 148);
(Z)-5-(3′-(2″-Fluorophenyl)benzylidene)1,2-dihydro-9-hydroxy-10-methoxy-2,2,4-trimethyl-5H-chromeno[3,4-f]quinoline (compound 149);
(Z)-5-(2′-chloro-3′-methylbenzylidene)1,2-dihydro-9-hydroxy-10-methoxy-2,2,4-trimethyl-5H-chromeno[3,4-f]quinoline (compound 150);
(Z)-5-(2′-(5′-Methyl-3′-(piperidinecarbonyl)furanylmethylidene))1,2-dihydro-9-hydroxy-10-methoxy-2,2,4-trimethyl-5H-chromeno[3,4-f]quinoline (compound 151);
(Z)-5-(2′-(5′-Methyl-3′-(piperidinecarbonyl)thienylmethylidene))1,2-dihydro-9-hydroxy-10-methoxy-2,2,4-trimethyl-5H-chromeno[3,4-f]quinoline (compound 152);
(Z)-5-(2′-(3′-Diethylcarbamoyl-1′,5′-dimethyl-1′H-pyrrolylmethylidene))1,2-dihydro-9-hydroxy-10-methoxy-2,2,4-trimethyl-5H-chromeno[3,4-f]quinoline (compound 153);
(Z)-5-(3′-Methyl-2′-(pyrrolidinecarbonyl)benzylidene)1,2-dihydro-9-hydroxy-10-methoxy-2,2,4-trimethyl-5H-chromeno[3,4-f]quinoline (compound 154);
(Z)-5-(3′-Bromo-2′-(pyrrolidinecarbonyl)benzylidene)1,2-dihydro-9-hydroxy-10-methoxy-2,2,4-trimethyl-5H-chromeno[3,4-f]quinoline (compound 155);
(Z)-5-(3′-Chloro-2′-(pyrrolidinecarbonyl)benzylidene)1,2-dihydro-9-hydroxy-10-methoxy-2,2,4-trimethyl-5H-chromeno[3,4-f]quinoline (compound 156);
(Z)-5-(2′-(3′-Hydroxymethylthienylmethylidene))1,2-dihydro-9-hydroxy-10-methoxy-2,2,4-trimethyl-5H-chromeno[3,4-f]quinoline (compound 157);
(Z)-5-(2′-(Piperidinecarbonyl)benzylidene)1,2-dihydro-9-hydroxy-10-methoxy-2,2,4-trimethyl-5H-chromeno[3,4-f]quinoline (compound 158);
(Z)-5-(2′-Hydroxymethylbenzylidene)1,2-dihydro-9-hydroxy-10-methoxy-2,2,4-trimethyl-5H-chromeno[3,4-f]quinoline (compound 159);
(Z)-5-(2′-(3′-(Hydroxymethyl)-5′-methylfuranylmethylidene))1,2-dihydro-9-hydroxy-10-methoxy-2,2,4-trimethyl-5H-chromeno[3,4-f]quinoline (compound 160);
(Z)-5-(2′-Fluoro-3′-hydroxymethylbenzylidene)1,2-dihydro-9-hydroxy-10-methoxy-2,2,4-trimethyl-5H-chromeno[3,4-f]quinoline (compound 161);
(Z)-5-(4′-Fluoro-2′-hydroxymethylbenzylidene)1,2-dihydro-9-hydroxy-10-methoxy-2,2,4-trimethyl-5H-chromeno[3,4-f]quinoline (compound 162);
(Z)-5-(3′-Bromo-2′-hydroxymethylbenzylidene)1,2-dihydro-9-hydroxy-10-methoxy-2,2,4-trimethyl-5H-chromeno[3,4-f]quinoline (compound 163);
(Z)-5-(5′-Bromo-2′-hydroxymethylbenzylidene)1,2-dihydro-9-hydroxy-10-methoxy-2,2,4-trimethyl-5H-chromeno[3,4-f]quinoline (compound 164);
(Z)-5-(2′-(3′-(Piperidinylmethyl)thienylmethylidene))1,2-dihydro-9-hydroxy-10-methoxy-2,2,4-trimethyl-5H-chromeno[3,4-f]quinoline (compound 165);
(Z)-5-(2′-(3′-(Dimethylaminomethyl)thienylmethylidene))1,2-dihydro-9-hydroxy-10-methoxy-2,2,4-trimethyl-5H-chromeno[3,4-f]quinoline (compound 166);
(Z)-5-(2′-(Diethylaminomethyl)-4′-fluorobenzylidene)1,2-dihydro-9-hydroxy-10-methoxy-2,2,4-trimethyl-5H-chromeno[3,4-f]quinoline (compound 167);
(Z)-5-(2′-(3′-Acetylthienylmethylidene))1,2-dihydro-9-hydroxy-10-methoxy-2,2,4-trimethyl-5H-chromeno[3,4-f]quinoline (compound 168);
(Z)-5-(2′-(3′-(1″-Hydroxy-1″-methylethyl)thienylmethylidene))1,2-dihydro-9-hydroxy-10-methoxy-2,2,4-trimethyl-5H-chromeno[3,4-f]quinoline (compound 169);
(Z)-5-(2′-(3′-Benzoylthienylmethylidene))1,2-dihydro-9-hydroxy-10-methoxy-2,2,4-trimethyl-5H-chromeno[3,4-f]quinoline (compound 170);
(±)-(Z)-5-(2′-(3′-(1″-Hydroxyethyl)thienylmethylidene))1,2-dihydro-9-hydroxy-10-methoxy-2,2,4-trimethyl-5H-chromeno[3,4-f]quinoline (compound 171);
(±)-(Z)-5-(2′-(3′-(1″-Hydroxy-1″-phenylmethyl)thienylmethylidene))1,2-dihydro-9-hydroxy-10-methoxy-2,2,4-trimethyl-5H-chromeno[3,4-f]quinoline (compound 172);
(Z)-5-(4′-Fluoro-2′-acetylbenzylidene)1,2-dihydro-9-hydroxy-10-methoxy-2,2,4-trimethyl-5H-chromeno[3,4-f]quinoline (compound 173);
(Z)-5-(2′-(3′-((E)-1″-Hydroxyiminoethyl)thienylmethylidene))1,2-dihydro-9-hydroxy-10-methoxy-2,2,4-trimethyl-5H-chromeno[3,4-f]quinoline (compound 174);
(Z)-5-(2′-(3′-((Z)-1″-Hydroxyiminoethyl)thienylmethylidene))1,2-dihydro-9-hydroxy-10-methoxy-2,2,4-trimethyl-5H-chromeno[3,4-f]quinoline (compound 175);
(Z)-5-(2′-(3′-((E)-1″-Methoxyiminoethyl)thienylmethylidene))1,2-dihydro-9-hydroxy-10-methoxy-2,2,4-trimethyl-5H-chromeno[3,4-f]quinoline (compound 176);
(Z)-5-(2′-(3′-((Z)-1″-Methoxyiminoethyl)thienylmethylidene))1,2-dihydro-9-hydroxy-10-methoxy-2,2,4-trimethyl-5H-chromeno[3,4-f]quinoline (compound 177);
(Z)-5-(2′-(3′-((E)-1″-Allyloxyiminoethyl)thienylmethylidene))1,2-dihydro-9-hydroxy-10-methoxy-2,2,4-trimethyl-5H-chromeno[3,4-f]quinoline (compound 178);
(Z)-5-(2′-(3′-((Z)-1″-Allyloxyiminoethyl)thienylmethylidene))1,2-dihydro-9-hydroxy-10-methoxy-2,2,4-trimethyl-5H-chromeno[3,4-f]quinoline (compound 179);
(Z)-5-(2′-(3′-((E)-1″-Phenoxyiminoethyl)thienylmethylidene))1,2-dihydro-9-hydroxy-10-methoxy-2,2,4-trimethyl-5H-chromeno[3,4-f]quinoline (compound 180);
(Z)-5-(2′-(3′-((Z)-1″-Phenoxyiminoethyl)thienylmethylidene))1,2-dihydro-9-hydroxy-10-methoxy-2,2,4-trimethyl-5H-chromeno[3,4-f]quinoline (compound 181);
(Z)-5-(2′-(3′-((E)-1″-Ethoxyiminoethyl)thienylmethylidene))1,2-dihydro-9-hydroxy-10-methoxy-2,2,4-trimethyl-5H-chromeno[3,4-f]quinoline (compound 182);
(Z)-5-(2′-(3′-((Z)-1″-Ethoxyiminoethyl)thienylmethylidene))1,2-dihydro-9-hydroxy-10-methoxy-2,2,4-trimethyl-5H-chromeno[3,4-f]quinoline (compound 183);
(Z)-5-(2′-(3′-((E)-(Carboxymethoxy)iminoethyl)thienylmethylidene))1,2-dihydro-9-hydroxy-10-methoxy-2,2,4-trimethyl-5H-chromeno[3,4-f]quinoline (compound 184);
(Z)-5-(2′-(3′-((E)-1″-tert-Butoxyiminoethyl)thienylmethylidene))1,2-dihydro-9-hydroxy-10-methoxy-2,2,4-trimethyl-5H-chromeno[3,4-f]quinoline (compound 185);
(Z)-5-(2′-(3′-((E)-1″-Benzyloxyiminoethyl)thienylmethylidene))1,2-dihydro-9-hydroxy-10-methoxy-2,2,4-trimethyl-5H-chromeno[3,4-f]quinoline (compound 186);
(Z)-5-(2′-(3′-((Z)-1″-Benzyloxyiminoethyl)thienylmethylidene))1,2-dihydro-9-hydroxy-10-methoxy-2,2,4-trimethyl-5H-chromeno[3,4-f]quinoline (compound 187);
(Z)-5-(2′-(3′-((E)-1″-(p-Nitrobenzyloxy)iminoethyl)thienylmethylidene))1,2-dihydro-9-hydroxy-10-methoxy-2,2,4-trimethyl-5H-chromeno[3,4-f]quinoline (compound 188);
(Z)-5-(2′-(3′-((Z)-1″-(p-Nitrobenzyloxy)iminoethyl)thienylmethylidene))1,2-dihydro-9-hydroxy-10-methoxy-2,2,4-trimethyl-5H-chromeno[3,4-f]quinoline (compound 189);
(Z)-5-(2′-(3′-((E)-Hydroxyiminomethyl)thienylmethylidene))1,2-dihydro-9-hydroxy-10-methoxy-2,2,4-trimethyl-5H-chromeno[3,4-f]quinoline (compound 190);
(Z)-5-(4′-Fluoro-(E)-2′-(hydroxyiminomethyl)benzylidene)1,2-dihydro-9-hydroxy-10-methoxy-2,2,4-trimethyl-5H-chromeno[3,4-f]quinoline (compound 191);
(Z)-5-((E)-2′-(Hydroxyiminomethyl)benzylidene)1,2-dihydro-9-hydroxy-10-methoxy-2,2,4-trimethyl-5H-chromeno[3,4-f]quinoline (compound 192);
(Z)-5-(2′-(3′-(Methoxymeththienylmethylidene))1,2-dihydro-9-hydroxy-10-methoxy-2,2,4-trimethyl-5H-chromeno[3,4-f]quinoline (compound 193);
(Z)-5-(2′-(3′-(Methoxymethoxymethyl)thienylmethylidene))1,2-dihydro-9-hydroxy-10-methoxy-2,2,4-trimethyl-5H-chromeno[3,4-f]quinoline (compound 194);
(Z)-5-(2′-(3′-(Prop-2″-enyloxy methyl)thienylmethylidene))1,2-dihydro-9-hydroxy-10-methoxy-2,2,4-trimethyl-5H-chromeno[3,4-f]quinoline (compound 195);
(Z)-5-(2′-(3′-(Prop-2″-ynloxymethyl)thienylmethylidene))1,2-dihydro-9-hydroxy-10-methoxy-2,2,4-trimethyl-5H-chromeno[3,4-f]quinoline (compound 196);
(Z)-5-(4′-Fluoro-2′-(methoxymethoxymethyl)benzylidene)1,2-dihydro-9-hydroxy-10-methoxy-2,2,4-trimethyl-5H-chromeno[3,4-f]quinoline (compound 197);
(Z)-5-(2′-(Methoxymethoxymethyl)benzylidene)1,2-dihydro-9-hydroxy-10-methoxy-2,2,4-trimethyl-5H-chromeno[3,4-f]quinoline (compound 198);
(±)-(Z)-5-(2′-(3′-(1″-Hydroxybut-3″-enyl)thienylmethylidene))1,2-dihydro-9-hydroxy-10-methoxy-2,2,4-trimethyl-5H-chromeno[3,4-f]quinoline (compound 199);
(+)-(Z)-5-(2′-(3′-(1″-Hydroxybut-3″-enyl)thienylmethylidene))1,2-dihydro-9-hydroxy-10-methoxy-2,2,4-trimethyl-5H-chromeno[3,4-f]quinoline (compound 200);
(−)-(Z)-5-(2′-(3′-(1″-Hydroxybut-3″-enyl)thienylmethylidene))1,2-dihydro-9-hydroxy-10-methoxy-2,2,4-trimethyl-5H-chromeno[3,4-f]quinoline (compound 201);
(±)-(Z)-5-(2′-(3′-(1″-Hydroxy-2″,2″,2″-trifluoroethyl)thienylmethylidene))-1,2-dihydro-9-hydroxy-10-methoxy-2,2,4-trimethyl-5H-chromeno[3,4-f]quinoline (compound 202);
(+)-(Z)-5-(2′-(3′-(1″-Hydroxy-2″,2″,2″-trifluoroethyl)thienylmethylidene))-1,2-dihydro-9-hydroxy-10-methoxy-2,2,4-trimethyl-5H-chromeno[3,4-f]quinoline (compound 203);
(−)-(Z)-5-(2′-(3′-(1″-Hydroxy-2″,2″,2″-trifluoroethyl)thienylmethylidene))1,2-dihydro-9-hydroxy-10-methoxy-2,2,4-trimethyl-5H-chromeno[3,4-f]quinoline (compound 204);
(±)-(Z)-5-(2′-(3′-(1″-Hydroxyprop-2″-ynyl)thienylmethylidene))1,2-dihydro-9-hydroxy-10-methoxy-2,2,4-trimethyl-5H-chromeno[3,4-f]quinoline (compound 205);
(±)-(Z)-5-(4′-Fluoro-2′-(2″,2″,2″-trifluoro-1″-hydroxyethyl)benzylidene)1,2-dihydro-9-hydroxy-10-methoxy-2,2,4-trimethyl-5H-chromeno[3,4-f]quinoline (compound 206);
(±)-(Z)-5-(2′-(3′-(Hydroxythien-3″-ylmethyl)thienylmethylidene))1,2-dihydro-9-hydroxy-10-methoxy-2,2,4-trimethyl-5H-chromeno[3,4-f]quinoline (compound 207);
(±)-(Z)-5-(2′-(3′-((4″-Fluorophenyl)hydroxymethyl)thienylmethylidene))1,2-dihydro-9-hydroxy-10-methoxy-2,2,4-trimethyl-5H-chromeno[3,4-f]quinoline (compound 208);
(±)-(Z)-5-(2′-(3′-(1″-Hydroxyallyl)thienylmethylidene))1,2-dihydro-9-hydroxy-10-methoxy-2,2,4-trimethyl-5H-chromeno[3,4-f]quinoline (compound 209);
(±)-(Z)-5-(2′-(3′-(Cyclohexylhydroxymethyl)thienylmethylidene))1,2-dihydro-9-hydroxy-10-methoxy-2,2,4-trimethyl-5H-chromeno[3,4-f]quinoline (compound 210);
(±)-(Z)-5-(2′-(3′-(1″-Hydroxy-2″-phenylethyl)thienylmethylidene))1,2-dihydro-9-hydroxy-10-methoxy-2,2,4-trimethyl-5H-chromeno[3,4-f]quinoline (compound 211);
(±)-(Z)-5-(2′-(3′-(Hydroxythien-2″-ylmethyl)thienylmethylidene))1,2-dihydro-9-hydroxy-10-methoxy-2,2,4-trimethyl-5H-chromeno[3,4-f]quinoline (compound 212);
(Z)-5-(2′-(3′-Acryloylthienylmethylidene))1,2-dihydro-9-hydroxy-10-methoxy-2,2,4-trimethyl-5H-chromeno[3,4-f]quinoline (compound 213);
(Z)-5-(2′-(3′-(4″-Fluorobenzoyl)thienylmethylidene))1,2-dihydro-9-hydroxy-10-methoxy-2,2,4-trimethyl-5H-chromeno[3,4-f]quinoline (compound 214);
(Z)-5-(2′-(3′-(Thien-3″-ylcarbonyl)thienylmethylidene))1,2-dihydro-9-hydroxy-10-methoxy-2,2,4-trimethyl-5H-chromeno[3,4-f]quinoline (compound 215);
(Z)-5-(2′-(3′-(Cyclohexanecarbonyl)thienylmethylidene))1,2-dihydro-9-hydroxy-10-methoxy-2,2,4-trimethyl-5H-chromeno[3,4-f]quinoline (compound 216);
(Z)-5-(2′-(3′-(But-3″-enoyl)thienylmethylidene))1,2-dihydro-9-hydroxy-10-methoxy-2,2,4-trimethyl-5H-chromeno[3,4-f]quinoline (compound 217);
(Z)-5-(2′-(3′-(Aminomethyl)thienylmethylidene))1,2-dihydro-9-hydroxy-10-methoxy-2,2,4-trimethyl-5H-chromeno[3,4-f]quinoline (compound 218);
(Z)-5-(2′-(3′-(Phenylaminomethyl)thienylmethylidene))1,2-dihydro-9-hydroxy-10-methoxy-2,2,4-trimethyl-5H-chromeno[3,4-f]quinoline (compound 219);
(Z)-5-(2′-(3′-(Prop-2″-ynylaminomethyl)thienylmethylidene))1,2-dihydro-9-hydroxy-10-methoxy-2,2,4-trimethyl-5H-chromeno[3,4-f]quinoline (compound 220);
(Z)-5-(2′-(3′-((2″,2″,2″-Trifluoroethylamino)methyl)thienylmethylidene))1,2-dihydro-9-hydroxy-10-methoxy-2,2,4-trimethyl-5H-chromeno[3,4-f]quinoline (compound 221);
(Z)-5-(2′-(3′-(Cyclopropylaminomethyl)thienylmethylidene))1,2-dihydro-9-hydroxy-10-methoxy-2,2,4-trimethyl-5H-chromeno[3,4-f]quinoline (compound 222);
(Z)-5-(2′-(3′-(1″-Butylaminomethyl)thienylmethylidene))1,2-dihydro-9-hydroxy-10-methoxy-2,2,4-trimethyl-5H-chromeno[3,4-f]quinoline (compound 223);
(Z)-5-(2′-(3′-(2″-Hydroxyethoxymethyl)thienylmethylidene))1,2-dihydro-9-hydroxy-10-methoxy-2,2,4-trimethyl-5H-chromeno[3,4-f]quinoline (compound 224);
(Z)-5-(2′-(3′-Isopropenylthienylmethylidene))1,2-dihydro-9-hydroxy-10-methoxy-2,2,4-trimethyl-5H-chromeno[3,4-f]quinoline (compound 225);
(Z)-5-(2′-(3′-Formylthienylmethylidene))1,2-dihydro-9-hydroxy-10-methoxy-2,2,4-trimethyl-5H-chromeno[3,4-f]quinoline (compound 226);
(Z)-5-(2′-(3′-(Methoxyethoxymethoxymethyl)thienylmethylidene))1,2-dihydro-9-hydroxy-10-methoxy-2,2,4-trimethyl-5H-chromeno[3,4-f]quinoline (compound 227);
(Z)-5-(2′-(3′-(Trifluoroacetyl)thienylmethylidene))1,2-dihydro-9-hydroxy-10-methoxy-2,2,4-trimethyl-5H-chromeno[3,4-f]quinoline (compound 228);
(Z)-5-(2′-(3′-(2″,2″,2″-Trifluoro-1″-hydroxy-1″-(trifluoromethyl)ethyl)-thienylmethylidene))1,2-dihydro-9-hydroxy-10-methoxy-2,2,4-trimethyl-5H-chromeno[3,4-f]-quinoline (compound 229);
(Z)-5-(4′-Fluoro-2′-formylbenzylidene)1,2-dihydro-9-hydroxy-10-methoxy-2,2,4-trimethyl-5H-chromeno[3,4-f]quinoline (compound 230);
(Z)-5-(2′-(3′-Cyanothienylmethylidene))1,2-dihydro-9-hydroxy-10-methoxy-2,2,4-trimethyl-5H-chromeno[3,4-f]quinoline (compound 231);
(Z)-5-(2′-(3′-Carbamoylthienylmethylidene))1,2-dihydro-9-hydroxy-10-methoxy-2,2,4-trimethyl-5H-chromeno[3,4-f]quinoline (compound 232);
(Z)-5-(4′-Fluoro-2′-vinylbenzylidene)1,2-dihydro-9-hydroxy-10-methoxy-2,2,4-trimethyl-5H-chromeno[3,4-f]quinoline (compound 233);
(Z)-5-(4′-Fluoro-2′-(acetoxymethyl)benzylidene)1,2-dihydro-9-hydroxy-10-methoxy-2,2,4-trimethyl-5H-chromeno[3,4-f]quinoline (compound 234);
(Z)-5-(2′-Formylbenzylidene)1,2-dihydro-9-hydroxy-10-methoxy-2,2,4-trimethyl-5H-chromeno[3,4-f]quinoline (compound 235);
(Z)-5-(2′-Vinylbenzylidene)1,2-dihydro-9-hydroxy-10-methoxy-2,2,4-trimethyl-5H-chromeno[3,4-f]quinoline (compound 236);
(Z)-5-(2′-(3′-(But-2″-ynloxymethyl)thienylmethylidene))1,2-dihydro-9-hydroxy-10-methoxy-2,2,4-trimethyl-5H-chromeno[3,4-f]quinoline (compound 237);
(Z)-5-(2′-(3′-(2″-(E)-Cyanovinyl)thienylmethylidene))1,2-dihydro-9-hydroxy-10-methoxy-2,2,4-trimethyl-5H-chromeno[3,4-f]quinoline (compound 238);
(Z)-5-(2′-(3′-(Ethoxycarbonylmethoxymethyl)thienylmethylidene))1,2-dihydro-9-hydroxy-10-methoxy-2,2,4-trimethyl-5H-chromeno[3,4-f]quinoline (compound 239);
(Z)-5-(2′-(3′-(Carboxymethoxymethyl)thienylmethylidene))1,2-dihydro-9-hydroxy-10-methoxy-2,2,4-trimethyl-5H-chromeno[3,4-f]quinoline (compound 240);
(Z)-5-(2′-(3′-Vinylthienylmethylidene))1,2-dihydro-9-hydroxy-10-methoxy-2,2,4-trimethyl-5H-chromeno[3,4-f]quinoline (compound 241);
(±)-(Z)-5-(2′-(3′-(1″-Methoxy-2″,2″,2″-trifluoroethyl)thienylmethylidene))-1,2-dihydro-9-hydroxy-10-methoxy-2,2,4-trimethyl-5H-chromeno[3,4-f]quinoline (compound 242);
(Z)-5-(2′-(3′-(2″,2″,2″-Trifluoro-1″-methoxy-1″-(trifluoromethyl)ethyl)-thienylmethylidene))1,2-dihydro-9-hydroxy-10-methoxy-2,2,4-trimethyl-5H-chromeno[3,4-f]-quinoline (compound 243);
(Z)-5-(4′-Hydroxymethylbenzylidene)1,2-dihydro-9-hydroxy-10-methoxy-2,2,4-trimethyl-5H-chromeno[3,4-f]quinoline (compound 244);
(Z)-5-(2′-(3′-(1″-Hydroxy-1″-(thien-3′″-yl)ethyl)thienylmethylidene))1,2-dihydro-9-hydroxy-10-methoxy-2,2,4-trimethyl-5H-chromeno[3,4-f]quinoline (compound 245);
(Z)-5-(2′-(3′-(2″-Methoxycarbonylvinyl)thienylmethylidene))1,2-dihydro-9-hydroxy-10-methoxy-2,2,4-trimethyl-5H-chromeno[3,4-f]quinoline (compound 246);
(Z)-5-(2′-(3′-Hydroxymethylpyridinylmethylidene))1,2-dihydro-9-hydroxy-10-methoxy-2,2,4-trimethyl-5H-chromeno[3,4-f]quinoline (compound 247);
(Z)-5-(2′-(3′-(Hydroxyethylcarbamoyl)thienylmethylidene))1,2-dihydro-9-hydroxy-10-methoxy-2,2,4-trimethyl-5H-chromeno[3,4-f]quinoline (compound 248);
(Z)-5-(2′-(3′-Ethylcarbamoylthienylmethylidene))1,2-dihydro-9-hydroxy-10-methoxy-2,2,4-trimethyl-5H-chromeno[3,4-f]quinoline (compound 249);
(Z)-5-(2′-(3′-((R)-2″-(Carbomethoxy)pyrrolidinecarbonyl)-thienylmethylidene))1,2-dihydro-9-hydroxy-10-methoxy-2,2,4-trimethyl-5H-chromeno[3,4-f]quinoline (compound 250);
(Z)-5-(2′-(3′-(piperazinecarbonyl)thienylmethylidene))1,2-dihydro-9-hydroxy-10-methoxy-2,2,4-trimethyl-5H-chromeno[3,4-f]quinoline (compound 251);
(Z)-5-(2′-(3′-(4″-Oxo-piperidinecarbonyl)thienylmethylidene))1,2-dihydro-9-hydroxy-10-methoxy-2,2,4-trimethyl-5H-chromeno[3,4-f]quinoline (compound 252);
(Z)-5-(2′-(3′-(2″,2″,2″-Trifluoroethylcarbamoyl)thienylmethylidene)1,2-dihydro-9-hydroxy-10-methoxy-2,2,4-trimethyl-5H-chromeno[3,4-f]quinoline (compound 253);
(Z)-5-(2′-(3′-(4″-Hydroxypiperidinecarbonyl)thienylmethylidene))1,2-dihydro-9-hydroxy-10-methoxy-2,2,4-trimethyl-5H-chromeno[3,4-f]quinoline (compound 254);
(Z)-5-(2′-(3′-(4″-Methylpiperazinecarbonyl)thienylmethylidene))1,2-dihydro-9-hydroxy-10-methoxy-2,2,4-trimethyl-5H-chromeno[3,4-f]quinoline (compound 256);
(±)-(Z)-5-(2′-(3′-(1″-Hydroxy-4″,4″,4″-trifluorobut-2″-ynyl)thienyl-methylidene))1,2-dihydro-9-hydroxy-10-methoxy-2,2,4-trimethyl-5H-chromeno[3,4-f]quinoline (compound 257);
(Z)-5-(2′-(3′-(3″-Hydroxy-3″-phenylpropanoyl)thienylmethylidene))1,2-dihydro-9-hydroxy-10-methoxy-2,2,4-trimethyl-5H-chromeno[3,4-f]quinoline-(compound 258);
(Z)-5-(2′-(3′-(3″-Hydroxybutanoyl)thienylmethylidene))1,2-dihydro-9-hydroxy-10-methoxy-2,2,4-trimethyl-5H-chromeno[3,4-f]quinoline (compound 259); and
(Z)-5-(2′-(3′-(But-2″-enoyl)thienylmethylidene))1,2-dihydro-9-hydroxy-10-methoxy-2,2,4-trimethyl-5H-chromeno[3,4-f]quinoline (compound 260).

108. The compound of claim 1, selected from among:
(Z)-5-(3′-trifluoromethyl-benzylidene)-1,2-dihydro-9-hydroxy-10-methoxy-2,2,4-trimethyl-5H-chromeno[3,4-f]quinoline (compound 11);
(Z)-5-(2′-fluorobenzylidene)-1,2-dihydro-9-hydroxy-10-methoxy-2,2,4-trimethyl-5H-chromeno[3,4-f]quinoline (compound 12);
(Z)-5-(3′-chlorobenzylidene)-1,2-dihydro-9-hydroxy-10-methoxy-2,2,4-trimethyl-5H-chromeno[3,4-f]quinoline (compound 13);
(Z)-5-(2′,5′-dichlorobenzylidene)-1,2-dihydro-9-hydroxy-10-methoxy-2,2,4-trimethyl-5H-chromeno[3,4-f]quinoline (compound 14);
(Z)-5-(3′-methoxybenzylidene)-1,2-dihydro-9-hydroxy-10-methoxy-2,2,4-trimethyl-5H-chromeno[3,4-f]quinoline (compound 15);
(Z)-5-(2′-chlorobenzylidene)-1,2-dihydro-9-hydroxy-10-methoxy-2,2,4-trimethyl-5H-chromeno[3,4-f]quinoline (compound 16);
(Z)-5-(4′-chlorobenzylidene)-1,2-dihydro-9-hydroxy-10-methoxy-2,2,4-trimethyl-5H-chromeno[3,4-f]quinoline (compound 17);
(Z)-5-(3′-methylbenzylidene)-1,2-dihydro-9-hydroxy-10-methoxy-2,2,4-trimethyl-5H-chromeno[3,4-f]quinoline (compound 18);
(Z)-5-(4′-methylbenzylidene)-1,2-dihydro-9-hydroxy-10-methoxy-2,2,4-trimethyl-5H-chromeno[3,4-f]quinoline (compound 19);
(Z)-5-(4′-methoxybenzylidene)-1,2-dihydro-9-hydroxy-10-methoxy-2,2,4-trimethyl-5H-chromeno[3,4-f]quinoline (compound 20);
(Z)-5-(2′-bromobenzylidene)-1,2-dihydro-9-hydroxy-10-methoxy-2,2,4-trimethyl-5H-chromeno[3,4-f]quinoline (compound 21);
(Z)-5-(3′-trifluoromethoxybenzylidene)-1,2-dihydro-9-hydroxy-10-methoxy-2,2,4-trimethyl-5H-chromeno[3,4-f]quinoline (compound 22);
(Z)-5-(3′,5′-dichlorobenzylidene)-1,2-dihydro-9-hydroxy-10-methoxy-2,2,4-trimethyl-5H-chromeno[3,4-f]quinoline (compound 23);
(Z)-5-(3′-bromobenzylidene)-1,2-dihydro-9-hydroxy-10-methoxy-2,2,4-trimethyl-5H-chromeno[3,4-f]quinoline (compound 24);
(Z)-5-(2′-chloro-4′-fluorobenzylidene)-1,2-dihydro-9-hydroxy-10-methoxy-2,2,4-trimethyl-5H-chromeno[3,4-f]quinoline (compound 25);
(Z)-5-(4′-trifluoromethoxybenzylidene)-1,2-dihydro-9-hydroxy-10-methoxy-2,2,4-trimethyl-5H-chromeno[3,4-f]quinoline (compound 26);
(Z)-5-(3′-trifluoromethylthiobenzylidene)-1,2-dihydro-9-hydroxy-10-methoxy-2,2,4-trimethyl-5H-chromeno[3,4-f]quinoline (compound 27);
(Z)-5-(2′-fluoro-3′-methylbenzylidene)-1,2-dihydro-9-hydroxy-10-methoxy-2,2,4-trimethyl-5H-chromeno[3,4-f]quinoline (compound 28);
(Z)-5-(2′-fluoro-3′-trifluoromethylbenzylidene)-1,2-dihydro-9-hydroxy-10-methoxy-2,2,4-trimethyl-5H-chromeno[3,4-f]quinoline (compound 29);
(Z)-5-(3′,4′-dichlorobenzylidene)-1,2-dihydro-9-hydroxy-10-methoxy-2,2,4-trimethyl-5H-chromeno[3,4-f]quinoline (compound 30);
(Z)-5-(4′-chloro-3′-trifluoromethylbenzylidene)-1,2-dihydro-9-hydroxy-10-methoxy-2,2,4-trimethyl-5H-chromeno[3,4-f]quinoline (compound 31);
(Z)-5-(3′,5′-di(trifluoromethyl)benzylidene)-1,2-dihydro-9-hydroxy-10-methoxy-2,2,4-trimethyl-5H-chromeno[3,4-f]quinoline (compound 32);
(Z)-5-(3′-fluoro-5′-trifluoromethylbenzylidene)-1,2-dihydro-9-hydroxy-10-methoxy-2,2,4-trimethyl-5H-chromeno[3,4-f]quinoline (compound 33);
(Z)-5-(2′,4′,5′-trifluorobenzylidene)-1,2-dihydro-9-hydroxy-10-methoxy-2,2,4-trimethyl-5H-chromeno[3,4-f]quinoline (compound 34);
(Z)-5-(2′-methylbenzylidene)-1,2-dihydro-9-hydroxy-10-methoxy-2,2,4-trimethyl-5H-chromeno[3,4-f]quinoline (compound 35);
(Z)-5-(4′-ethylbenzylidene)-1,2-dihydro-9-hydroxy-10-methoxy-2,2,4-trimethyl-5H-chromeno[3,4-f]quinoline (compound 36);
(Z)-5-(5′-fluoro-2′-methylbenzylidene)-1,2-dihydro-9-hydroxy-10-methoxy-2,2,4-trimethyl-5H-chromeno[3,4-f]quinoline (compound 37);
(Z)-5-(2′-chloro-6′-fluorobenzylidene)1,2-dihydro-9-hydroxy-10-methoxy-2,2,4-trimethyl-5H-chromeno[3,4-f]quinoline (compound 38);
(Z)-5-(4′-isopropylbenzylidene)-1,2-dihydro-9-hydroxy-10-methoxy-2,2,4-trimethyl-5H-chromeno[3,4-f]quinoline (compound 39);
(Z)-5-(4′-bromobenzylidene)-1,2-dihydro-9-hydroxy-10-methoxy-2,2,4-trimethyl-5H-chromeno[3,4-f]quinoline (compound 40);
(Z)-5-(3′-fluoro-4′-methylbenzylidene)-1,2-dihydro-9-hydroxy-10-methoxy-2,2,4-trimethyl-5H-chromeno[3,4-f]quinoline (compound 41);
(Z)-5-(2′-methyl-3′-trifluoromethylbenzylidene)-1,2-dihydro-9-hydroxy-10-methoxy-2,2,4-trimethyl-5H-chromeno[3,4-f]quinoline (compound 43);
(Z)-5-(4′-benzyloxybenzylidene)-1,2-dihydro-9-hydroxy-10-methoxy-2,2,4-trimethyl-5H-chromeno[3,4-f]quinoline (compound 44);
(Z)-5-(2′-phenylbenzylidene)-1,2-dihydro-9-hydroxy-10-methoxy-2,2,4-trimethyl-5H-chromeno[3,4-f]quinoline (compound 46);
(Z)-5-(4′-phenylbenzylidene)-1,2-dihydro-9-hydroxy-10-methoxy-2,2,4-trimethyl-5H-chromeno[3,4-f]quinoline (compound 47);
(Z)-5-(3′-methyl-4′-fluorobenzylidene)-1,2-dihydro-9-hydroxy-10-methoxy-2,2,4-trimethyl-5H-chromeno[3,4-f]quinoline (compound 48);
(Z)-5-(4′-cyclohexylbenzylidene)-1,2-dihydro-9-hydroxy-10-methoxy-2,2,4-trimethyl-5H-chromeno[3,4-f]quinoline (compound 49);
(Z)-5-(2′-chloro-3′-trifluoromethylbenzylidene)-1,2-dihydro-9-hydroxy-10-methoxy-2,2,4-trimethyl-5H-chromeno[3,4-f]quinoline (compound 51);
(Z)-5-(3′-phenylbenzylidene)-1,2-dihydro-9-hydroxy-10-methoxy-2,2,4-trimethyl-5H-chromeno[3,4-f]quinoline (compound 52);
(Z)-5-(3′-chloro-4′-trifluoromethoxybenzylidene)-1,2-dihydro-9-hydroxy-10-methoxy-2,2,4-trimethyl-5H-chromeno[3,4-f]quinoline (compound 54);
(Z)-5-(2′,6′-difluoro-3′-chlorobenzylidene)-1,2-dihydro-9-hydroxy-10-methoxy-2,2,4-trimethyl-5H-chromeno[3,4-f]quinoline (compound 55);
(Z)-5-(2′-chloro-3′,6′-difluorobenzylidene)-1,2-dihydro-9-hydroxy-10-methoxy-2,2,4-trimethyl-5H-chromeno[3,4-f]quinoline (compound 56);
(Z)-5-(4′-methyl-3′-trifluoromethylbenzylidene)-1,2-dihydro-9-hydroxy-10-methoxy-2,2,4-trimethyl-5H-chromeno[3,4-f]quinoline (compound 58);
(Z)-5-(2′-fluoro-4′-chlorobenzylidene)-1,2-dihydro-9-hydroxy-10-methoxy-2,2,4-trimethyl-5H-chromeno[3,4-f]quinoline (compound 59);
(Z)-5-(2′,3′-difluoro-4′-methylbenzylidene)-1,2-dihydro-9-hydroxy-10-methoxy-2,2,4-trimethyl-5H-chromeno[3,4-f]quinoline (compound 60);
(Z)-5-(2′,3′,5′,6′-tetrafluoro-4′-trifluoromethylbenzylidene)-1,2-dihydro-9-hydroxy-10-methoxy-2,2,4-trimethyl-5H-chromeno[3,4-f]quinoline (compound 61);
(Z)-5-(4′-vinylbenzylidene)-1,2-dihydro-9-hydroxy-10-methoxy-2,2,4-trimethyl-5H-chromeno[3,4-f]quinoline (compound 63);
(Z)-5-(2′-Chloro-6′-fluoro-5′-methylbenzylidene)-1,2-dihydro-9-hydroxy-10-methoxy-2,2,4-trimethyl-5H-chromeno[3,4-f]quinoline (compound 64);
(Z)-5-(2′-trifluoromethoxybenzylidene)-1,2-dihydro-9-hydroxy-10-methoxy-2,2,4-trimethyl-5H-chromeno[3,4-f]quinoline (compound 65);
(Z)-5-(2′-trifluoromethylthiobenzylidene)-1,2-dihydro-9-hydroxy-10-methoxy-2,2,4-trimethyl-5H-chromeno[3,4-f]quinoline (compound 66);
(Z)-5-(3′,4′-methylenedioxybenzylidene)-1,2-dihydro-9-hydroxy-10-methoxy-2,2,4-trimethyl-5H-chromeno[3,4-f]quinoline (compound 67);
(Z)-5-(3′-chloro-2′-fluorobenzylidene)-1,2-dihydro-9-hydroxy-10-methoxy-2,2,4-trimethyl-5H-chromeno[3,4-f]quinoline (compound 68);
(Z)-5-(4′-(4″-methylbenzyloxy)benzylidene)-1,2-dihydro-9-hydroxy-10-methoxy-2,2,4-trimethyl-5H-chromeno[3,4-f]quinoline (compound 70);
(Z)-5-(3′,5′-di-tert-butylbenzylidene)-1,2-dihydro-9-hydroxy-10-methoxy-2,2,4-trimethyl-5H-chromeno[3,4-f]quinoline (compound 71);
(Z)-5-(3′-(2″,2″-difluoroethoxy)benzylidene)-1,2-dihydro-9-hydroxy-10-methoxy-2,2,4-trimethyl-5H-chromeno[3,4-f]quinoline (compound 72);
(Z)-5-(2′,5′-dimethylbenzylidene)-1,2-dihydro-9-hydroxy-10-methoxy-2,2,4-trimethyl-5H-chromeno[3,4-f]quinoline (compound 73);
(Z)-5-(3′-(3″-thienyl)benzylidene)-1,2-dihydro-9-hydroxy-10-methoxy-2,2,4-trimethyl-5H-chromeno[3,4-f]quinoline (compound 74);
(Z)-5-(2′-diethylaminocarbonylbenzylidene)-1,2-dihydro-9-hydroxy-10-methoxy-2,2,4-trimethyl-5H-chromeno[3,4-f]quinoline (compound 75);
(Z)-5-(3′-(4″,4″,4″-trifluorobutoxy)benzylidene)-1,2-dihydro-9-hydroxy-10-methoxy-2,2,4-trimethyl-5H-chromeno[3,4-f]quinoline (compound 76);
(Z)-5-(3′-(2″,4″-difluorophenyl)benzylidene)-1,2-dihydro-9-hydroxy-10-methoxy-2,2,4-trimethyl-5H-chromeno[3,4-f]quinoline (compound 77);
(Z)-5-(3′-(3″-pyridyl)benzylidene)-1,2-dihydro-9-hydroxy-10-methoxy-2,2,4-trimethyl-5H-chromeno[3,4-f]quinoline (compound 78);
(Z)-5-(2′-(3″-formylphenyl)benzylidene)-1,2-dihydro-9-hydroxy-10-methoxy-2,2,4-trimethyl-5H-chromeno[3,4-f]quinoline (compound 79);
(Z)-5-(3′,5′-dimethylbenzylidene)-1,2-dihydro-9-hydroxy-10-methoxy-2,2,4-trimethyl-5H-chromeno[3,4-f]quinoline (compound 80);
(Z)-5-(3′,4′-dimethylbenzylidene)-1,2-dihydro-9-hydroxy-10-methoxy-2,2,4-trimethyl-5H-chromeno[3,4-f]quinoline (compound 81);
(Z)-5-(2′-(diethylamino)carbonyl-6′-fluorobenzylidene)-1,2-dihydro-9-hydroxy-10-methoxy-2,2,4-trimethyl-5H-chromeno[3,4-f]quinoline (compound 82);
(Z)-5-(2′-(diethylamino)carbonyl-4′-fluorobenzylidene)-1,2-dihydro-9-hydroxy-10-methoxy-2,2,4-trimethyl-5H-chromeno[3,4-f]quinoline (compound 83);
(Z)-5-(2′-(methylbenzylamino)carbonyl-6′-fluorobenzylidene)-1,2-dihydro-9-hydroxy-10-methoxy-2,2,4-trimethyl-5H-chromeno[3,4-f]quinoline (compound 84);
(Z)-5-(2′-(dimethylamino)carbonyl-5′-bromo-benzylidene)-1,2-dihydro-9-hydroxy-10-methoxy-2,2,4-trimethyl-5H-chromeno[3,4-f]quinoline (compound 85);
(Z)-5-(3′-(2″-fluoroethoxy)benzylidene)-1,2-dihydro-9-hydroxy-10-methoxy-2,2,4-trimethyl-5H-chromeno[3,4-f]quinoline (compound 86);
(Z)-5-(3′-(2″,2″,3″,3″-tetrafluoropropoxy)benzylidene)-1,2-dihydro-9-hydroxy-10-methoxy-2,2,4-trimethyl-5H-chromeno[3,4-f]quinoline (compound 87);
(Z)-5-(3′-(4″-fluorobenzyloxy)benzylidene)-1,2-dihydro-9-hydroxy-10-methoxy-2,2,4-trimethyl-5H-chromeno[3,4-f]quinoline (compound 88);
(Z)-5-(3′-(2″-fluorobenzyloxy)benzylidene)-1,2-dihydro-9-hydroxy-10-methoxy-2,2,4-trimethyl-5H-chromeno[3,4-f]quinoline (compound 89);
(Z)-5-(2′-(pyrrolidinecarbonyl)benzylidene)-1,2-dihydro-9-hydroxy-10-methoxy-2,2,4-trimethyl-5H-chromeno[3,4-f]quinoline (compound 90);
(Z)-5-(2′-(pyrrolidinecarbonyl)-5′-bromobenzylidene)-1,2-dihydro-9-hydroxy-10-methoxy-2,2,4-trimethyl-5H-chromeno[3,4-f]quinoline (compound 91);
(Z)-5-(2′-(dimethylaminocarbonyl)-4′-fluorobenzylidene)-1,2-dihydro-9-hydroxy-10-methoxy-2,2,4-trimethyl-5H-chromeno[3,4-f]quinoline (compound 92);
(Z)-5-(2′-(pyrrolidinecarbonyl)-5′-methylbenzylidene)-1,2-dihydro-9-hydroxy-10-methoxy-2,2,4-trimethyl-5H-chromeno[3,4-f]quinoline (compound 93);
(Z)-5-(2′-(pyrrolidinecarbonyl)-4′-fluorobenzylidene)-1,2-dihydro-9-hydroxy-10-methoxy-2,2,4-trimethyl-5H-chromeno[3,4-f]quinoline (compound 94);
(Z)-5-(3′-(4″-fluorophenoxy)benzylidene)-1,2-dihydro-9-hydroxy-10-methoxy-2,2,4-trimethyl-5H-chromeno[3,4-f]quinoline (compound 95);
(Z)-5-(2′-(morpholinocarbonyl)-4′-fluorobenzylidene)-1,2-dihydro-9-hydroxy-10-methoxy-2,2,4-trimethyl-5H-chromeno[3,4-f]quinoline (compound 96);
(Z)-5-(8′-(6′-fluoro-benzo-1′,3′-dioxan-methylidiene))-1,2-dihydro-9-hydroxy-10-methoxy-2,2,4-trimethyl-5H-chromeno[3,4-f]quinoline (compound 97);
(Z)-5-(2′-dimethylaminocarbonyl-3′-methoxybenzylidene)-1,2-dihydro-9-hydroxy-10-methoxy-2,2,4-trimethyl-5H-chromeno[3,4-f]quinoline (compound 98);
(Z)-5-(2′-(4″-methylpiperazinecarbonyl)-4′-fluorobenzylidene)-1,2-dihydro-9-hydroxy-10-methoxy-2,2,4-trimethyl-5H-chromeno[3,4-f]quinoline (compound 99);
(Z)-5-(2′-methyl-3′-phenylbenzylidene)-1,2-dihydro-9-hydroxy-10-methoxy-2,2,4-trimethyl-5H-chromeno[3,4-f]quinoline (compound 100);
(Z)-5-(3′,5′-di-methoxybenzylidene)-1,2-dihydro-9-hydroxy-10-methoxy-2,2,4-trimethyl-5H-chromeno[3,4-f]quinoline (compound 101);
(Z)-5-(2′-(piperidinecarbonyl)-4′-fluorobenzylidene)-1,2-dihydro-9-hydroxy-10-methoxy-2,2,4-trimethyl-5H-chromeno[3,4-f]quinoline (compound 102);
(Z)-5-(2′-dimethylaminosulphonyl-4′-fluorobenzylidene)-1,2-dihydro-9-hydroxy-10-methoxy-2,2,4-trimethyl-5H-chromeno[3,4-f]quinoline (compound 103);
(Z)-5-(3′-phenoxybenzylidene)-1,2-dihydro-9-hydroxy-10-methoxy-2,2,4-trimethyl-5H-chromeno[3,4-f]quinoline (compound 104);
(Z)-5-(2′-(ethylmethylamino)carbonyl-4′-fluorobenzylidene)-1,2-dihydro-9-hydroxy-10-methoxy-2,2,4-trimethyl-5H-chromeno[3,4-f]quinoline (compound 105);
(Z)-5-(2′-(cyclohexylmethylamino)carbonyl-4′-fluorobenzylidene)-1,2-dihydro-9-hydroxy-10-methoxy-2,2,4-trimethyl-5H-chromeno[3,4-f]quinoline (compound 106);
(Z)-5-(2′-cyanobenzylidene)-1,2-dihydro-9-hydroxy-10-methoxy-2,2,4-trimethyl-5H-chromeno[3,4-f]quinoline (compound 107);
(Z)-5-(2′,3′,5′,6′-tetrafluoro-4′-methoxybenzylidene)-1,2-dihydro-9-hydroxy-10-methoxy-2,2,4-trimethyl-5H-chromeno[3,4-f]quinoline (compound 108);
(Z)-5-(3′-hydroxybenzylidene)-1,2-dihydro-9-hydroxy-10-methoxy-2,2,4-trimethyl-5H-chromeno[3,4-f]quinoline (compound 109);
(Z)-5-(2′-(piperidinesulphonyl)-4′-fluorobenzylidene)-1,2-dihydro-9-hydroxy-10-methoxy-2,2,4-trimethyl-5H-chromeno[3,4-f]quinoline (compound 110);
(Z)-5-(1′-napthylmethylidene)-1,2-dihydro-9-hydroxy-10-methoxy-2,2,4-trimethyl-5H-chromeno[3,4-f]quinoline (compound 111);
(Z)-5-(3′-methyl-4′-methoxybenzylidene)-1,2-dihydro-9-hydroxy-10-methoxy-2,2,4-trimethyl-5H-chromeno[3,4-f]quinoline (compound 112);
(Z)-5-(2′,5′-dimethoxybenzylidene)-1,2-dihydro-9-hydroxy-10-methoxy-2,2,4-trimethyl-5H-chromeno[3,4-f]quinoline (compound 113);
(Z)-5-(2′,3′-methylenedioxybenzylidene)-1,2-dihydro-9-hydroxy-10-methoxy-2,2,4-trimethyl-5H-chromeno[3,4-f]quinoline (compound 114);
(Z)-5-(2′,3′-ethylenedioxybenzylidene)-1,2-dihydro-9-hydroxy-10-methoxy-2,2,4-trimethyl-5H-chromeno[3,4-f]quinoline (compound 115);
(Z)-5-(4′-hydroxybenzylidene)-1,2-dihydro-9-hydroxy-10-methoxy-2,2,4-trimethyl-5H-chromeno[3,4-f]quinoline (compound 116);
(Z)-5-(2′-cyano-3′-methylbenzylidene)-1,2-dihydro-9-hydroxy-10-methoxy-2,2,4-trimethyl-5H-chromeno[3,4-f]quinoline (compound 117);
(Z)-5-(3′-chloro-2′-cyanobenzylidene)-1,2-dihydro-9-hydroxy-10-methoxy-2,2,4-trimethyl-5H-chromeno[3,4-f]quinoline (compound 118);
(Z)-5-(5′-bromo-2′-cyano-benzylidene)-1,2-dihydro-9-hydroxy-10-methoxy-2,2,4-trimethyl-5H-chromeno[3,4-f]quinoline (compound 119);
(Z)-5-(8′-(6′-chloro-benzo-1′,3′-dioxan-methylidene)-1,2-dihydro-9-hydroxy-10-methoxy-2,2,4-trimethyl-5H-chromeno[3,4-f]quinoline (compound 120);
(Z)-5-(2′-chloro-3′,4′-dimethoxybenzylidene)-1,2-dihydro-9-hydroxy-10-methoxy-2,2,4-trimethyl-5H-chromeno[3,4-f]quinoline (compound 121);
(Z)-5-(2′-cyano-3′-fluorobenzylidene)-1,2-dihydro-9-hydroxy-10-methoxy-2,2,4-trimethyl-5H-chromeno[3,4-f]quinoline (compound 122);
(Z)-5-(8′-(6′-methyl-benzo-1′,3′-dioxan-methylidene))-1,2-dihydro-9-hydroxy-10-methoxy-2,2,4-trimethyl-5H-chromeno[3,4-f]quinoline (compound 123);
(Z)-5-(2′-cyano-5′-methylbenzylidene)-1,2-dihydro-9-hydroxy-10-methoxy-2,2,4-trimethyl-5H-chromeno[3,4-f]quinoline (compound 124);
(Z)-5-(8′-(5′,6′-difluoro-benzo-1′,3′-dioxan-methylidene))-1,2-dihydro-9-hydroxy-10-methoxy-2,2,4-trimethyl-5H-chromeno[3,4-f]quinoline (compound 125);
(Z)-5-(3′-(3″,5″-dichlorophenoxy)benzylidene)-1,2-dihydro-9-hydroxy-10-methoxy-2,2,4-trimethyl-5H-chromeno[3,4-f]quinoline (compound 126);
(Z)-5-(3′-(4″-methoxyphenoxy)benzylidene)-1,2-dihydro-9-hydroxy-10-methoxy-2,2,4-trimethyl-5H-chromeno[3,4-f]quinoline (compound 127);
(Z)-5-(3′-(3″,4″-dichlorophenoxy)benzylidene)-1,2-dihydro-9-hydroxy-10-methoxy-2,2,4-trimethyl-5H-chromeno[3,4-f]quinoline (compound 128);
(Z)-5-(3′-(4″-methylphenoxy)benzylidene)-1,2-dihydro-9-hydroxy-10-methoxy-2,2,4-trimethyl-5H-chromeno[3,4-f]quinoline (compound 129);
(Z)-5-(3′-(4″-chlorophenoxy)benzylidene)-1,2-dihydro-9-hydroxy-10-methoxy-2,2,4-trimethyl-5H-chromeno[3,4-f]quinoline (compound 130); and
(Z)-5-(3′-(3″-trifluoromethoxyphenoxy)benzylidene)-1,2-dihydro-9-hydroxy-10-methoxy-2,2,4-trimethyl-5H-chromeno[3,4-f]quinoline (compound 131).

109. The compound of claim 1 selected from among:
(Z)-5-(2′-fluorobenzylidene)-1,2-dihydro-9-hydroxy-10-methoxy-2,2,4-trimethyl-5H-chromeno[3,4-f]quinoline (compound 12);
(Z)-5-(3′-methylbenzylidene)-1,2-dihydro-9-hydroxy-10-methoxy-2,2,4-trimethyl-5H-chromeno[3,4-f]quinoline (compound 18);
(Z)-5-(2′-bromobenzylidene)-1,2-dihydro-9-hydroxy-10-methoxy-2,2,4-trimethyl-5H-chromeno[3,4-f]quinoline (compound 21);
(Z)-5-(3′-trifluoromethylthiobenzylidene)-1,2-dihydro-9-hydroxy-10-methoxy-2,2,4-trimethyl-5H-chromeno[3,4-f]quinoline (compound 27);
(Z)-5-(2′-fluoro-3′-methylbenzylidene)-1,2-dihydro-9-hydroxy-10-methoxy-2,2,4-trimethyl-5H-chromeno[3,4-f]quinoline (compound 28);
(Z)-5-(2′-fluoro-3′-trifluoromethylbenzylidene)-1,2-dihydro-9-hydroxy-10-methoxy-2,2,4-trimethyl-5H-chromeno[3,4-f]quinoline (compound 29);
(Z)-5-(2′,4′,5′-trifluorobenzylidene)-1,2-dihydro-9-hydroxy-10-methoxy-2,2,4-trimethyl-5H-chromeno[3,4-f]quinoline (compound 34);
(Z)-5-(2′-trifluoromethoxybenzylidene)-1,2-dihydro-9-hydroxy-10-methoxy-2,2,4-trimethyl-5H-chromeno[3,4-f]quinoline (compound 65);
(Z)-5-(3′-(4″-fluorophenoxy)benzylidene)-1,2-dihydro-9-hydroxy-10-methoxy-2,2,4-trimethyl-5H-chromeno[3,4-f]quinoline (compound 95);
(Z)-5-(8′-(6′-fluoro-benzo-1′,3′-dioxan-methylidene))-1,2-dihydro-9-hydroxy-10-methoxy-2,2,4-trimethyl-5H-chromeno[3,4-f]quinoline (compound 97);
(Z)-5-(3′-phenoxybenzylidene)-1,2-dihydro-9-hydroxy-10-methoxy-2,2,4-trimethyl-5H-chromeno[3,4-f]quinoline (compound 104);
(Z)-5-(2′-cyanobenzylidene)-1,2-dihydro-9-hydroxy-10-methoxy-2,2,4-trimethyl-5H-chromeno[3,4-f]quinoline (compound 107);
(Z)-5-(3′-hydroxybenzylidene)-1,2-dihydro-9-hydroxy-10-methoxy-2,2,4-trimethyl-5H-chromeno[3,4-f]quinoline (compound 109);
(Z)-5-(2′,3′-methylenedioxybenzylidene)-1,2-dihydro-9-hydroxy-10-methoxy-2,2,4-trimethyl-5H-chromeno[3,4-f]quinoline (compound 114);
(Z)-5-(2′-cyano-3′-methylbenzylidene)-1,2-dihydro-9-hydroxy-10-methoxy-2,2,4-trimethyl-5H-chromeno[3,4-f]quinoline (compound 117);
(Z)-5-(3′-chloro-2′-cyanobenzylidene)-1,2-dihydro-9-hydroxy-10-methoxy-2,2,4-trimethyl-5H-chromeno[3,4-f]quinoline (compound 118);
(Z)-5-(8′-(6′-chloro-benzo-1′,3′-dioxan-methylidene)-1,2-dihydro-9-hydroxy-10-methoxy-2,2,4-trimethyl-5H-chromeno[3,4-f]quinoline (compound 120); and
(Z)-5-(2′-cyano-3′-fluorobenzylidene)-1,2-dihydro-9-hydroxy-10-methoxy-2,2,4-trimethyl-5H-chromeno[3,4-f]quinoline (compound 122).

110. The compound of claim 1, selected from among:
(Z)-5-(2′,5′-dichlorobenzylidene)-1,2-dihydro-9-hydroxy-10-methoxy-2,2,4-trimethyl-5H-chromeno[3,4-f]quinoline (compound 14);
(Z)-5-(4′-chlorobenzylidene)-1,2-dihydro-9-hydroxy-10-methoxy-2,2,4-trimethyl-5H-chromeno[3,4-f]quinoline (compound 17);
(Z)-5-(2′-bromobenzylidene)-1,2-dihydro-9-hydroxy-10-methoxy-2,2,4-trimethyl-5H-chromeno[3,4-f]quinoline (compound 21);
(Z)-5-(3′-fluoro-5′-trifluoromethylbenzylidene)-1,2-dihydro-9-hydroxy-10-methoxy-2,2,4-trimethyl-5H-chromeno[3,4-f]quinoline (compound 33);
(Z)-5-(3′-chloro-4′-trifluoromethoxybenzylidene)-1,2-dihydro-9-hydroxy-10-methoxy-2,2,4-trimethyl-5H-chromeno[3,4-f]quinoline (compound 54);
(Z)-5-(4′-methyl-3′-trifluoromethylbenzylidene)-1,2-dihydro-9-hydroxy-10-methoxy-2,2,4-trimethyl-5H-chromeno[3,4-f]quinoline (compound 58);
(Z)-5-(3′-(3″,5″-dichlorophenoxy)benzylidene)-1,2-dihydro-9-hydroxy-10-methoxy-2,2,4-trimethyl-5H-chromeno[3,4-f]quinoline (compound 126); and
(Z)-5-(3′-(3″,4″-dichlorophenoxy)benzylidene)-1,2-dihydro-9-hydroxy-10-methoxy-2,2,4-trimethyl-5H-chromeno[3,4-f]quinoline (compound 128).

111. The compound of claim 1, selected from among:
(Z)-5-(4′-(2″-fluorophenyl)-benzylidene)1,2-dihydro-9-hydroxy-10-methoxy-2,2,4-trimethyl-5H-chromeno[3,4-f]-quinoline (compound 148);
(Z)-5-(3′-(2″-Fluorophenyl)benzylidene)1,2-dihydro-9-hydroxy-10-methoxy-2,2,4-trimethyl-5H-chromeno[3,4-f]quinoline (compound 149);
(Z)-5-(2′-chloro-3′-methylbenzylidene)1,2-dihydro-9-hydroxy-10-methoxy-2,2,4-trimethyl-5H-chromeno[3,4-f]quinoline (compound 150);
(Z)-5-(3′-Methyl-2′-(pyrrolidinecarbonyl)benzylidene)1,2-dihydro-9-hydroxy-10-methoxy-2,2,4-trimethyl-5H-chromeno[3,4-f]quinoline (compound 154);
(Z)-5-(3′-Bromo-2′-(pyrrolidinecarbonyl)benzylidene)1,2-dihydro-9-hydroxy-10-methoxy-2,2,4-trimethyl-5H-chromeno[3,4-f]quinoline (compound 155);
(Z)-5-(3′-Chloro-2′-(pyrrolidinecarbonyl)benzylidene)1,2-dihydro-9-hydroxy-10-methoxy-2,2,4-trimethyl-5H-chromeno[3,4-f]quinoline (compound 156);
(Z)-5-(2′-(Piperidinecarbonyl)benzylidene)1,2-dihydro-9-hydroxy-10-methoxy-2,2,4-trimethyl-5H-chromeno[3,4-f]quinoline (compound 158);
(Z)-5-(2′-Hydroxymethylbenzylidene)1,2-dihydro-9-hydroxy-10-methoxy-2,2,4-trimethyl-5H-chromeno[3,4-f]quinoline (compound 159);
(Z)-5-(2′-Fluoro-3′-hydroxymethylbenzylidene)1,2-dihydro-9-hydroxy-10-methoxy-2,2,4-trimethyl-5H-chromeno[3,4-f]quinoline (compound 161);
(Z)-5-(4′-Fluoro-2′-hydroxymethylbenzylidene)1,2-dihydro-9-hydroxy-10-methoxy-2,2,4-trimethyl-5H-chromeno[3,4-f]quinoline (compound 162);
(Z)-5-(3′-Bromo-2′-hydroxymethylbenzylidene)1,2-dihydro-9-hydroxy-10-methoxy-2,2,4-trimethyl-5H-chromeno[3,4-f]quinoline (compound 163);
(Z)-5-(5′-Bromo-2′-hydroxymethylbenzylidene)1,2-dihydro-9-hydroxy-10-methoxy-2,2,4-trimethyl-5H-chromeno[3,4-f]quinoline (compound 164);
(Z)-5-(2′-(Diethylaminomethyl)-4′-fluorobenzylidene)1,2-dihydro-9-hydroxy-10-methoxy-2,2,4-trimethyl-5H-chromeno[3,4-f]quinoline (compound 167);
(Z)-5-(4′-Fluoro-2′-acetylbenzylidene)1,2-dihydro-9-hydroxy-10-methoxy-2,2,4-trimethyl-5H-chromeno[3,4-f]quinoline (compound 173);
(Z)-5-(4′-Fluoro-(E)-2′-(hydroxyiminomethyl)benzylidene)1,2-dihydro-9-hydroxy-10-methoxy-2,2,4-trimethyl-5H-chromeno[3,4-f]quinoline (compound 191);
(Z)-5-((E)-2′-(Hydroxyiminomethyl)benzylidene)1,2-dihydro-9-hydroxy-10-methoxy-2,2,4-trimethyl-5H-chromeno[3,4-f]quinoline (compound 192);
(Z)-5-(4′-Fluoro-2′-(methoxymethoxymethyl)benzylidene)1,2-dihydro-9-hydroxy-10-methoxy-2,2,4-trimethyl-5H-chromeno[3,4-f]quinoline (compound 197);
(Z)-5-(2′-(Methoxymethoxymethyl)benzylidene)1,2-dihydro-9-hydroxy-10-methoxy-2,2,4-trimethyl-5H-chromeno[3,4-f]quinoline (compound 198);
(±)-(Z)-5-(4′-Fluoro-2′-(2″,2″,2″-trifluoro-1″-hydroxyethyl)benzylidene)1,2-dihydro-9-hydroxy-10-methoxy-2,2,4-trimethyl-5H-chromeno[3,4-f]quinoline (compound 206);
(±)-(Z)-5-(2′-(3′-(Hydroxythien-3″-ylmethyl)thienylmethylidene))1,2-dihydro-9-hydroxy-10-methoxy-2,2,4-trimethyl-5H-chromeno[3,4-f]quinoline (compound 207);
(±)-(Z)-5-(2′-(3′-((4″-Fluorophenyl)hydroxymethyl)thienylmethylidene))1,2-dihydro-9-hydroxy-10-methoxy-2,2,4-trimethyl-5H-chromeno[3,4-f]quinoline (compound 208);
(±)-(Z)-5-(2′-(3′-(1″-Hydroxyallyl)thienylmethylidene))1,2-dihydro-9-hydroxy-10-methoxy-2,2,4-trimethyl-5H-chromeno[3,4-f]quinoline (compound 209);
(±)-(Z)-5-(2′-(3′-(Cyclohexylhydroxymethyl)thienylmethylidene))1,2-dihydro-9-hydroxy-10-methoxy-2,2,4-trimethyl-5H-chromeno[3,4-f]quinoline (compound 210);
(±)-(Z)-5-(2′-(3′-(1″-Hydroxy-2″-phenylethyl)thienylmethylidene))1,2-dihydro-9-hydroxy-10-methoxy-2,2,4-trimethyl-5H-chromeno[3,4-f]quinoline (compound 211);
(±)-(Z)-5-(2′-(3′-(Hydroxythien-2″-ylmethyl)thienylmethylidene))1,2-dihydro-9-hydroxy-10-methoxy-2,2,4-trimethyl-5H-chromeno[3,4-f]quinoline (compound 212);
(Z)-5-(2′-(3′-Acryloylthienylmethylidene))1,2-dihydro-9-hydroxy-10-methoxy-2,2,4-trimethyl-5H-chromeno[3,4-f]quinoline (compound 213);
(Z)-5-(2′-(3′-(4″-Fluorobenzoyl)thienylmethylidene))1,2-dihydro-9-hydroxy-10-methoxy-2,2,4-trimethyl-5H-chromeno[3,4-f]quinoline (compound 214);
(Z)-5-(2′-(3′-(Thien-3″-ylcarbonyl)thienylmethylidene))1,2-dihydro-9-hydroxy-10-methoxy-2,2,4-trimethyl-5H-chromeno[3,4-f]quinoline (compound 215);
(Z)-5-(2′-(3′-(Cyclohexanecarbonyl)thienylmethylidene))1,2-dihydro-9-hydroxy-10-methoxy-2,2,4-trimethyl-5H-chromeno[3,4-f]quinoline (compound 216);
(Z)-5-(2′-(3′-(But-3″-enoyl)thienylmethylidene))1,2-dihydro-9-hydroxy-10-methoxy-2,2,4-trimethyl-5H-chromeno[3,4-f]quinoline (compound 217);
(Z)-5-(2′-(3′-(Aminomethyl)thienylmethylidene))1,2-dihydro-9-hydroxy-10-methoxy-2,2,4-trimethyl-5H-chromeno[3,4-f]quinoline (compound 218);
(Z)-5-(2′-(3′-(Phenylaminomethyl)thienylmethylidene))1,2-dihydro-9-hydroxy-10-methoxy-2,2,4-trimethyl-5H-chromeno[3,4-f]quinoline (compound 219);
(Z)-5-(2′-(3′-(Prop-2″-ynylaminomethyl)thienylmethylidene))1,2-dihydro-9-hydroxy-10-methoxy-2,2,4-trimethyl-5H-chromeno[3,4-f]quinoline (compound 220);
(Z)-5-(2′-(3′-((2″,2″,2″-Trifluoroethylamino)methyl)thienylmethylidene))1,2-dihydro-9-hydroxy-10-methoxy-2,2,4-trimethyl-5H-chromeno[3,4-f]quinoline (compound 221);
(Z)-5-(2′-(3′-(Cyclopropylaminomethyl)thienylmethylidene))1,2-dihydro-9-hydroxy-10-methoxy-2,2,4-trimethyl-5H-chromeno[3,4-f]quinoline (compound 222);
(Z)-5-(2′-(3′-(1″-Butylaminomethyl)thienylmethylidene))1,2-dihydro-9-hydroxy-10-methoxy-2,2,4-trimethyl-5H-chromeno[3,4-f]quinoline (compound 223);
(Z)-5-(2′-(3′-(2″-Hydroxyethoxymethyl)thienylmethylidene))1,2-dihydro-9-hydroxy-10-methoxy-2,2,4-trimethyl-5H-chromeno[3,4-f]quinoline (compound 224);
(Z)-5-(2′-(3′-Isopropenylthienylmethylidene))1,2-dihydro-9-hydroxy-10-methoxy-2,2,4-trimethyl-5H-chromeno[3,4-f]quinoline (compound 225);
(Z)-5-(2′-(3′-Formylthienylmethylidene))1,2-dihydro-9-hydroxy-10-methoxy-2,2,4-trimethyl-5H-chromeno[3,4-f]quinoline (compound 226);
(Z)-5-(2′-(3′-(Methoxyethoxymethoxymethyl)thienylmethylidene))1,2-dihydro-9-hydroxy-10-methoxy-2,2,4-trimethyl-5H-chromeno[3,4-f]quinoline (compound 227);
(Z)-5-(2′-(3′-(Trifluoroacetyl)thienylmethylidene))1,2-dihydro-9-hydroxy-10-methoxy-2,2,4-trimethyl-5H-chromeno[3,4-f]quinoline (compound 228);
(Z)-5-(2′-(3′-(2″,2″,2″-Trifluoro-1″-hydroxy-1″-(trifluoromethyl)ethyl)-thienylmethylidene))1,2-dihydro-9-hydroxy-10-methoxy-2,2,4-trimethyl-5H-chromeno[3,4-f]quinoline (compound 229);
(Z)-5-(4′-Fluoro-2′-formylbenzylidene)1,2-dihydro-9-hydroxy-10-methoxy-2,2,4-trimethyl-5H-chromeno[3,4-f]quinoline (compound 230);
(Z)-5-(4′-Fluoro-2′-vinylbenzylidene)1,2-dihydro-9-hydroxy-10-methoxy-2,2,4-trimethyl-5H-chromeno[3,4-f]quinoline (compound 233);
(Z)-5-(4′-Fluoro-2′-(acetoxymethyl)benzylidene)1,2-dihydro-9-hydroxy-10-methoxy-2,2,4-trimethyl-5H-chromeno[3,4-f]quinoline (compound 234);
(Z)-5-(2′-Formylbenzylidene)1,2-dihydro-9-hydroxy-10-methoxy-2,2,4-trimethyl-5H-chromeno[3,4-f]quinoline (compound 235); and
(Z)-5-(2′-Vinylbenzylidene)1,2-dihydro-9-hydroxy-10-methoxy-2,2,4-trimethyl-5H-chromeno[3,4-f]quinoline (compound 236).

112. The compound of claim 1, selected from among:
(Z)-5-(2′-Hydroxymethyl-benzylidene)1,2-dihydro-9-hydroxy-10-methoxy-2,2,4-trimethyl-5H-chromeno[3,4-f]quinoline (compound 159);
(Z)-5-(2′-Fluoro-3′-hydroxymethylbenzylidene)1,2-dihydro-9-hydroxy-10-methoxy-2,2,4-trimethyl-5H-chromeno[3,4-f]quinoline (compound 161);
(Z)-5-(2′-(Methoxymethoxymethyl)benzylidene)1,2-dihydro-9-hydroxy-10-methoxy-2,2,4-trimethyl-5H-chromeno[3,4-f]quinoline (compound 198);
(±)-(Z)-5-(4′-Fluoro-2′-(2″,2″,2″-trifluoro-1″-hydroxyethyl)benzylidene)1,2-dihydro-9-hydroxy-10-methoxy-2,2,4-trimethyl-5H-chromeno[3,4-f]quinoline (compound 206); and
(Z)-5-(4′-Fluoro-2′-vinylbenzylidene)1,2-dihydro-9-hydroxy-10-methoxy-2,2,4-trimethyl-5H-chromeno[3,4-f]quinoline (compound 233).

113. The compound of claim 1, selected from among:
(Z)-5-(2′-chloro-3′-methylbenzylidene)1,2-dihydro-9-hydroxy-10-methoxy-2,2,4-trimethyl-5H-chromeno[3,4-f]quinoline (compound 150);
(Z)-5-(3′-Chloro-2′-(pyrrolidinecarbonyl)benzylidene)1,2-dihydro-9-hydroxy-10-methoxy-2,2,4-trimethyl-5H-chromeno[3,4-f]quinoline (compound 156);
(Z)-5-(5′-Bromo-2′-hydroxymethylbenzylidene)1,2-dihydro-9-hydroxy-10-methoxy-2,2,4-trimethyl-5H-chromeno[3,4-f]quinoline (compound 164); and
(Z)-5-(4′-Fluoro-2′-(methoxymethoxymethyl)benzylidene)1,2-dihydro-9-hydroxy-10-methoxy-2,2,4-trimethyl-5H-chromeno[3,4-f]quinoline (compound 197).

114. The compound of claim 1, selected from among:
(Z)-5-(2′-(6′-methyl-pyridinylmethylidiene))-1,2-dihydro-9-hydroxy-10-methoxy-2,2,4-trimethyl-5H-chromeno[3,4-f]quinoline (compound 42);
(Z)-5-(2′-(3′-(dimethylaminocarbonyl)furanylmethylidene))-1,2-dihydro-9-hydroxy-10-methoxy-2,2,4-trimethyl-5H-chromeno[3,4-f]quinoline (compound 62);
(Z)-5-(2′-(3′-(dimethylaminocarbonyl)thienylmethylidene))-1,2-dihydro-9-hydroxy-10-methoxy-2,2,4-trimethyl-5H-chromeno[3,4-f]quinoline (compound 132);
(Z)-5-(2′-(3′-(ethylmethylaminocarbonyl)thienylmethylidene))-1,2-dihydro-9-hydroxy-10-methoxy-2,2,4-trimethyl-5H-chromeno[3,4-f]quinoline (compound 134);
(Z)-5-(2′-(3′-(morpholinocarbonyl)thienylmethylidene)-1,2-dihydro-9-hydroxy-10-methoxy-2,2,4-trimethyl-5H-chromeno[3,4-f]quinoline (compound 135);
(Z)-5-(2′-(3′-(cyclohexylmethylaminocarbonyl)thienylmethylidene))-1,2-dihydro-9-hydroxy-10-methoxy-2,2,4-trimethyl-5H-chromeno[3,4-f]quinoline (compound 136);
(Z)-5-(2′-(3′-(pyrrolidinocarbonyl)thienylmethylidene))-1,2-dihydro-9-hydroxy-10-methoxy-2,2,4-trimethyl-5H-chromeno[3,4-f]quinoline (compound 137);
(Z)-5-(2′-(3′-(di(methoxyethyl)aminocarbonyl)thienylmethylidene)-1,2-dihydro-9-hydroxy-10-methoxy-2,2,4-trimethyl-5H-chromeno[3,4-f]quinoline (compound 138);
(Z)-5-(2′-(3′-(allylmethylaminocarbonyl)thienylmethylidene))-1,2-dihydro-9-hydroxy-10-methoxy-2,2,4-trimethyl-5H-chromeno[3,4-f]quinoline (compound 139);
(Z)-5-(2′-(3′-(piperidinocarbonyl)thienylmethylidene))-1,2-dihydro-9-hydroxy-10-methoxy-2,2,4-trimethyl-5H-chromeno[3,4-f]quinoline (compound 140);
(Z)-5-(2′-(3′-piperidinecarbonyl-4″-(1,3-dioxan)thienylmethylidene)-1,2-dihydro-9-hydroxy-10-methoxy-2,2,4-trimethyl-5H-chromeno[3,4-f]quinoline (compound 141);
(Z)-5-(2′-(5′-(diethylaminocarbonyl)thienylmethylidene))-1,2-dihydro-9-hydroxy-10-methoxy-2,2,4-trimethyl-5H-chromeno[3,4-f]quinoline (compound 142);
(Z)-5-(2′-(5′-(pyrrolidinocarbonyl)thienylmethylidene))-1,2-dihydro-9-hydroxy-10-methoxy-2,2,4-trimethyl-5H-chromeno[3,4-f]quinoline (compound 143);
(Z)-5-(2′-(5′-(2″-methylpyrrolidinocarbonyl)thienylmethylidene))-1,2-dihydro-9-hydroxy-10-methoxy-2,2,4-trimethyl-5H-chromeno[3,4-f]quinoline (compound 144);
(Z)-5-(2′-(5′-(morpholinocarbonyl)thienylmethylidene))-1,2-dihydro-9-hydroxy-10-methoxy-2,2,4-trimethyl-5H-chromeno[3,4-f]quinoline (compound 145);
(Z)-5-(2′-(3′-dimethylaminocarbonyl-5′-methylfuranylmethylidene))-1,2-dihydro-9-hydroxy-10-methoxy-2,2,4-trimethyl-5H-chromeno[3,4-f]quinoline (compound 146); and
(Z)-5-(2′-(3′-cyclohexylmethylaminocarbonyl-5′-methylfuranyl-methylidene))-1,2-dihydro-9-hydroxy-10-methoxy-2,2,4-trimethyl-5H-chromeno[3,4-f]quinoline (compound 147).

115. The compound of claim 1, selected from among:
(Z)-5-(2′-(5′-Methyl-3′-(piperidinecarbonyl)furanylmethylidene))1,2-dihydro-9-hydroxy-10-methoxy-2,2,4-trimethyl-5H-chromeno[3,4-f]quinoline (compound 151);
(Z)-5-(2′-(5′-Methyl-3′-(piperidinecarbonyl)thienylmethylidene))1,2-dihydro-9-hydroxy-10-methoxy-2,2,4-trimethyl-5H-chromeno[3,4-f]quinoline (compound 152);
(Z)-5-(2′-(3′-Diethylcarbamoyl-1′,5′-dimethyl-1′H-pyrrolylmethylidene))1,2-dihydro-9-hydroxy-10-methoxy-2,2,4-trimethyl-5H-chromeno[3,4-f]quinoline (compound 153);
(Z)-5-(2′-(3′-Hydroxymethylthienylmethylidene))1,2-dihydro-9-hydroxy-10-methoxy-2,2,4-trimethyl-5H-chromeno[3,4-f]quinoline (compound 157);
(Z)-5-(2′-(3′-(Hydroxymethyl)-5′-methylfuranylmethylidene))1,2-dihydro-9-hydroxy-10-methoxy-2,2,4-trimethyl-5H-chromeno[3,4-f]quinoline (compound 160);
(Z)-5-(2′-(3′-(Piperidinylmethyl)thienylmethylidene))1,2-dihydro-9-hydroxy-10-methoxy-2,2,4-trimethyl-5H-chromeno[3,4-f]quinoline (compound 165);
(Z)-5-(2′-(3′-(Dimethylaminomethyl)thienylmethylidene))1,2-dihydro-9-hydroxy-10-methoxy-2,2,4-trimethyl-5H-chromeno[3,4-f]quinoline (compound 166);
(Z)-5-(2′-(3′-Acetylthienylmethylidene))1,2-dihydro-9-hydroxy-10-methoxy-2,2,4-trimethyl-5H-chromeno[3,4-f]quinoline (compound 168);
(Z)-5-(2′-(3′-(1″-Hydroxy-1″-methylethyl)thienylmethylidene))1,2-dihydro-9-hydroxy-10-methoxy-2,2,4-trimethyl-5H-chromeno[3,4-f]quinoline (compound 169);
(Z)-5-(2′-(3′-Benzoylthienylmethylidene))1,2-dihydro-9-hydroxy-10-methoxy-2,2,4-trimethyl-5H-chromeno[3,4-f]quinoline (compound 170);
(±)-(Z)-5-(2′-(3′-(1″-Hydroxyethyl)thienylmethylidene))1,2-dihydro-9-hydroxy-10-methoxy-2,2,4-trimethyl-5H-chromeno[3,4-f]quinoline (compound 171);
(±)-(Z)-5-(2′-(3′-(1″-Hydroxy-1″-phenylmethyl)thienylmethylidene))1,2-dihydro-9-hydroxy-10-methoxy-2,2,4-trimethyl-5H-chromeno[3,4-f]quinoline (compound 172);
(Z)-5-(2′-(3′-((E)-1″-Hydroxyiminoethyl)thienylmethylidene))1,2-dihydro-9-hydroxy-10-methoxy-2,2,4-trimethyl-5H-chromeno[3,4-f]quinoline (compound 174);
(Z)-5-(2′-(3′-((Z)-1″-Hydroxyiminoethyl)thienylmethylidene))1,2-dihydro-9-hydroxy-10-methoxy-2,2,4-trimethyl-5H-chromeno[3,4-f]quinoline (compound 175);
(Z)-5-(2′-(3′-((E)-1″-Methoxyiminoethyl)thienylmethylidene))1,2-dihydro-9-hydroxy-10-methoxy-2,2,4-trimethyl-5H-chromeno[3,4-f]quinoline (compound 176);
(Z)-5-(2′-(3′-((Z)-1″-Methoxyiminoethyl)thienylmethylidene))1,2-dihydro-9-hydroxy-10-methoxy-2,2,4-trimethyl-5H-chromeno[3,4-f]quinoline (compound 177);
(Z)-5-(2′-(3′-((E)-1″-Allyloxyiminoethyl)thienylmethylidene))1,2-dihydro-9-hydroxy-10-methoxy-2,2,4-trimethyl-5H-chromeno[3,4-f]quinoline (compound 178);
(Z)-5-(2′-(3′-((Z)-1″-Allyloxyiminoethyl)thienylmethylidene))1,2-dihydro-9-hydroxy-10-methoxy-2,2,4-trimethyl-5H-chromeno[3,4-f]quinoline (compound 179);
(Z)-5-(2′-(3′-((E)-1″-Phenoxyiminoethyl)thienylmethylidene))1,2-dihydro-9-hydroxy-10-methoxy-2,2,4-trimethyl-5H-chromeno[3,4-f]quinoline (compound 180);
(Z)-5-(2′-(3′-((Z)-1″-Phenoxyiminoethyl)thienylmethylidene))1,2-dihydro-9-hydroxy-10-methoxy-2,2,4-trimethyl-5H-chromeno[3,4-f]quinoline (compound 181);
(Z)-5-(2′-(3′-((E)-1″-Ethoxyiminoethyl)thienylmethylidene))1,2-dihydro-9-hydroxy-10-methoxy-2,2,4-trimethyl-5H-chromeno[3,4-f]quinoline (compound 182);
(Z)-5-(2′-(3′-((Z)-1″-Ethoxyiminoethyl)thienylmethylidene))1,2-dihydro-9-hydroxy-10-methoxy-2,2,4-trimethyl-5H-chromeno[3,4-f]quinoline (compound 183);
(Z)-5-(2′-(3′-((E)-(Carboxymethoxy)iminoethyl)thienylmethylidene))1,2-dihydro-9-hydroxy-10-methoxy-2,2,4-trimethyl-5H-chromeno[3,4-f]quinoline (compound 184);
(Z)-5-(2′-(3′-((E)-1″-tert-Butoxyiminoethyl)thienylmethylidene))1,2-dihydro-9-hydroxy-10-methoxy-2,2,4-trimethyl-5H-chromeno[3,4-f]quinoline (compound 185);
(Z)-5-(2′-(3′-((E)-1″-Benzyloxyiminoethyl)thienylmethylidene))1,2-dihydro-9-hydroxy-10-methoxy-2,2,4-trimethyl-5H-chromeno[3,4-f]quinoline (compound 186);
(Z)-5-(2′-(3′-((Z)-1″-Benzyloxyiminoethyl)thienylmethylidene))1,2-dihydro-9-hydroxy-10-methoxy-2,2,4-trimethyl-5H-chromeno[3,4-f]quinoline (compound 187);
(Z)-5-(2′-(3′-((E)-1″-(p-Nitrobenzyloxy)iminoethyl)thienylmethylidene))1,2-dihydro-9-hydroxy-10-methoxy-2,2,4-trimethyl-5H-chromeno[3,4-f]quinoline (compound 188);
(Z)-5-(2′-(3′-((Z)-1″-(p-Nitrobenzyloxy)iminoethyl)thienylmethylidene))1,2-dihydro-9-hydroxy-10-methoxy-2,2,4-trimethyl-5H-chromeno[3,4-f]quinoline (compound 189);
(Z)-5-(2′-(3′-((E)-Hydroxyiminomethyl)thienylmethylidene))1,2-dihydro-9-hydroxy-10-methoxy-2,2,4-trimethyl-5H-chromeno[3,4-f]quinoline (compound 190);
(Z)-5-(2′-(3′-Methoxymethylthienylmethylidene))1,2-dihydro-9-hydroxy-10-methoxy-2,2,4-trimethyl-5H-chromeno[3,4-f]quinoline (compound 193);
(Z)-5-(2′-(3′-(Methoxymethoxymethyl)thienylmethylidene))1,2-dihydro-9-hydroxy-10-methoxy-2,2,4-trimethyl-5H-chromeno[3,4-f]quinoline (compound 194);
(Z)-5-(2′-(3′-(Prop-2″-enyloxy methyl)thienylmethylidene))1,2-dihydro-9-hydroxy-10-methoxy-2,2,4-trimethyl-5H-chromeno[3,4-f]quinoline (compound 195);
(Z)-5-(2′-(3′-(Prop-2″-ynloxymethyl)thienylmethylidene))1,2-dihydro-9-hydroxy-10-methoxy-2,2,4-trimethyl-5H-chromeno[3,4-f]quinoline (compound 196);
(±)-(Z)-5-(2′-(3′-(1″-Hydroxybut-3″-enyl)thienylmethylidene))1,2-dihydro-9-hydroxy-10-methoxy-2,2,4-trimethyl-5H-chromeno[3,4-f]quinoline (compound 199);
(+)-(Z)-5-(2′-(3′-(1″-Hydroxybut-3″-enyl)thienylmethylidene))1,2-dihydro-9-hydroxy-10-methoxy-2,2,4-trimethyl-5H-chromeno[3,4-f]quinoline (compound 200);
(−)-(Z)-5-(2′-(3′-(1″-Hydroxybut-3″-enyl)thienylmethylidene))1,2-dihydro-9-hydroxy-10-methoxy-2,2,4-trimethyl-5H-chromeno[3,4-f]quinoline (compound 201);
(±)-(Z)-5-(2′-(3′-(1″-Hydroxy-2″,2″,2″-trifluoroethyl)thienylmethylidene))-1,2-dihydro-9-hydroxy-10-methoxy-2,2,4-trimethyl-5H-chromeno[3,4-f]quinoline (compound 202);
(+)-(Z)-5-(2′-(3′-(1″-Hydroxy-2″,2″,2″-trifluoroethyl)thienylmethylidene))-1,2-dihydro-9-hydroxy-10-methoxy-2,2,4-trimethyl-5H-chromeno[3,4-f]quinoline (compound 203);
(−)-(Z)-5-(2′-(3′-(1″-Hydroxy-2″,2″,2″-trifluoroethyl)thienylmethylidene))1,2-dihydro-9-hydroxy-10-methoxy-2,2,4-trimethyl-5H-chromeno[3,4-f]quinoline (compound 204);
(±)-(Z)-5-(2′-(3′-(1″-Hydroxyprop-2″-ynyl)thienylmethylidene))1,2-dihydro-9-hydroxy-10-methoxy-2,2,4-trimethyl-5H-chromeno[3,4-f]quinoline (compound 205);
(±)-(Z)-5-(2′-(3′-(Hydroxythien-3″-ylmethyl)thienylmethylidene))1,2-dihydro-9-hydroxy-10-methoxy-2,2,4-trimethyl-5H-chromeno[3,4-f]quinoline (compound 207);
(±)-(Z)-5-(2′-(3′-((4″-Fluorophenyl)hydroxymethyl)thienylmethylidene))1,2-dihydro-9-hydroxy-10-methoxy-2,2,4-trimethyl-5H-chromeno[3,4-f]quinoline (compound 208);
(±)-(Z)-5-(2′-(3′-(1″-Hydroxyallyl)thienylmethylidene))1,2-dihydro-9-hydroxy-10-methoxy-2,2,4-trimethyl-5H-chromeno[3,4-f]quinoline (compound 209);
(±)-(Z)-5-(2′-(3′-(Cyclohexylhydroxymethyl)thienylmethylidene))1,2-dihydro-9-hydroxy-10-methoxy-2,2,4-trimethyl-5H-chromeno[3,4-f]quinoline (compound 210);
(±)-(Z)-5-(2′-(3′-(1″-Hydroxy-2″-phenylethyl)thienylmethylidene))1,2-dihydro-9-hydroxy-10-methoxy-2,2,4-trimethyl-5H-chromeno[3,4-f]quinoline (compound 211);
(±)-(Z)-5-(2′-(3′-(Hydroxythien-2″-ylmethyl)thienylmethylidene))1,2-dihydro-9-hydroxy-10-methoxy-2,2,4-trimethyl-5H-chromeno[3,4-f]quinoline (compound 212);
(Z)-5-(2′-(3′-Acryloylthienylmethylidene))1,2-dihydro-9-hydroxy-10-methoxy-2,2,4-trimethyl-5H-chromeno[3,4-f]quinoline (compound 213);
(Z)-5-(2′-(3′-(4″-Fluorobenzoyl)thienylmethylidene))1,2-dihydro-9-hydroxy-10-methoxy-2,2,4-trimethyl-5H-chromeno[3,4-f]quinoline (compound 214);
(Z)-5-(2′-(3′-(Thien-3″-ylcarbonyl)thienylmethylidene))1,2-dihydro-9-hydroxy-10-methoxy-2,2,4-trimethyl-5H-chromeno[3,4-f]quinoline (compound 215);
(Z)-5-(2′-(3′-(Cyclohexanecarbonyl)thienylmethylidene))1,2-dihydro-9-hydroxy-10-methoxy-2,2,4-trimethyl-5H-chromeno[3,4-f]quinoline (compound 216);
(Z)-5-(2′-(3′-(But-3″-enoyl)thienylmethylidene))1,2-dihydro-9-hydroxy-10-methoxy-2,2,4-trimethyl-5H-chromeno[3,4-f]quinoline (compound 217);
(Z)-5-(2′-(3′-(Aminomethyl)thienylmethylidene))1,2-dihydro-9-hydroxy-10-methoxy-2,2,4-trimethyl-5H-chromeno[3,4-f]quinoline (compound 218);
(Z)-5-(2′-(3′-(Phenylaminomethyl)thienylmethylidene))1,2-dihydro-9-hydroxy-10-methoxy-2,2,4-trimethyl-5H-chromeno[3,4-f]quinoline (compound 219);
(Z)-5-(2′-(3′-(Prop-2″-ynylaminomethyl)thienylmethylidene))1,2-dihydro-9-hydroxy-10-methoxy-2,2,4-trimethyl-5H-chromeno[3,4-f]quinoline (compound 220);
(Z)-5-(2′-(3′-((2″,2″,2″-Trifluoroethylamino)methyl)thienylmethylidene))1,2-dihydro-9-hydroxy-10-methoxy-2,2,4-trimethyl-5H-chromeno[3,4-f]quinoline (compound 221);
(Z)-5-(2′-(3′-(Cyclopropylaminomethyl)thienylmethylidene))1,2-dihydro-9-hydroxy-10-methoxy-2,2,4-trimethyl-5H-chromeno[3,4-f]quinoline (compound 222);
(Z)-5-(2′-(3′-(1″-Butylaminomethyl)thienylmethylidene))1,2-dihydro-9-hydroxy-10-methoxy-2,2,4-trimethyl-5H-chromeno[3,4-f]quinoline (compound 223);
(Z)-5-(2′-(3′-(2″-Hydroxyethoxymethyl)thienylmethylidene))1,2-dihydro-9-hydroxy-10-methoxy-2,2,4-trimethyl-5H-chromeno[3,4-f]quinoline (compound 224);
(Z)-5-(2′-(3′-Isopropenylthienylmethylidene))1,2-dihydro-9-hydroxy-10-methoxy-2,2,4-trimethyl-5H-chromeno[3,4-f]quinoline (compound 225);
(Z)-5-(2′-(3′-Formylthienylmethylidene))1,2-dihydro-9-hydroxy-10-methoxy-2,2,4-trimethyl-5H-chromeno[3,4-f]quinoline (compound 226);
(Z)-5-(2′-(3′-(Methoxyethoxymethoxymethyl)thienylmethylidene))1,2-dihydro-9-hydroxy-10-methoxy-2,2,4-trimethyl-5H-chromeno[3,4-f]quinoline (compound 227);
(Z)-5-(2′-(3′-(Trifluoroacetyl)thienylmethylidene))1,2-dihydro-9-hydroxy-10-methoxy-2,2,4-trimethyl-5H-chromeno[3,4-f]quinoline (compound 228);
(Z)-5-(2′-(3′-(2″,2″,2″-Trifluoro-1″-hydroxy-1″-(trifluoromethyl)ethyl)-thienylmethylidene))1,2-dihydro-9-hydroxy-10-methoxy-2,2,4-trimethyl-5H-chromeno[3,4-f]-quinoline (compound 229);
(Z)-5-(2′-(3′-(But-2″-ynloxymethyl)thienylmethylidene))1,2-dihydro-9-hydroxy-10-methoxy-2,2,4-trimethyl-5H-chromeno[3,4-f]quinoline (compound 237);
(Z)-5-(2′-(3′-(2″-(E)-Cyanovinyl)thienylmethylidene))1,2-dihydro-9-hydroxy-10-methoxy-2,2,4-trimethyl-5H-chromeno[3,4-f]quinoline (compound 238);
(Z)-5-(2′-(3′-(Ethoxycarbonylmethoxymethyl)thienylmethylidene))1,2-dihydro-9-hydroxy-10-methoxy-2,2,4-trimethyl-5H-chromeno[3,4-f]quinoline (compound 239);
(Z)-5-(2′-(3′-(Carboxymethoxymethyl)thienylmethylidene))1,2-dihydro-9-hydroxy-10-methoxy-2,2,4-trimethyl-5H-chromeno[3,4-f]quinoline (compound 240);
(Z)-5-(2′-(3′-Vinylthienylmethylidene))1,2-dihydro-9-hydroxy-10-methoxy-2,2,4-trimethyl-5H-chromeno[3,4-f]quinoline (compound 241);
(±)-(Z)-5-(2′-(3′-(1″-Methoxy-2″,2″,2″-trifluoroethyl)thienylmethylidene))-1,2-dihydro-9-hydroxy-10-methoxy-2,2,4-trimethyl-5H-chromeno[3,4-f]quinoline (compound 242);
(Z)-5-(2′-(3′-(2″,2″,2″-Trifluoro-1″-methoxy-1″-(trifluoromethyl)ethyl)-thienylmethylidene))1,2-dihydro-9-hydroxy-10-methoxy-2,2,4-trimethyl-5H-chromeno[3,4-f]-quinoline (compound 243);
(Z)-5-(4′-Hydroxymethylbenzylidene)1,2-dihydro-9-hydroxy-10-methoxy-2,2,4-trimethyl-5H-chromeno[3,4-f]quinoline (compound 244);
(Z)-5-(2′-(3′-(1″-Hydroxy-1″-(thien-3′″-yl)ethyl)thienylmethylidene))1,2-dihydro-9-hydroxy-10-methoxy-2,2,4-trimethyl-5H-chromeno[3,4-f]quinoline (compound 245);
(Z)-5-(2′-(3′-(2″-Methoxycarbonylvinyl)thienylmethylidene))1,2-dihydro-9-hydroxy-10-methoxy-2,2,4-trimethyl-5H-chromeno[3,4-f]quinoline (compound 246);
(Z)-5-(2′-(3′-Hydroxymethylpyridinylmethylidene))1,2-dihydro-9-hydroxy-10-methoxy-2,2,4-trimethyl-5H-chromeno[3,4-f]quinoline (compound 247);
(Z)-5-(2′-(3′-(Hydroxyethylcarbamoyl)thienylmethylidene))1,2-dihydro-9-hydroxy-10-methoxy-2,2,4-trimethyl-5H-chromeno[3,4-f]quinoline (compound 248);
(Z)-5-(2′-(3′-Ethylcarbamoylthienylmethylidene))1,2-dihydro-9-hydroxy-10-methoxy-2,2,4-trimethyl-5H-chromeno[3,4-f]quinoline (compound 249);
(Z)-5-(2′-(3′-((R)-2″-(Carbomethoxy)pyrrolidinecarbonyl)thienyl-methylidene))1,2-dihydro-9-hydroxy-10-methoxy-2,2,4-trimethyl-5H-chromeno[3,4-f]quinoline (compound 250);
(Z)-5-(2′-(3′-(piperazinecarbonyl)thienylmethylidene))1,2-dihydro-9-hydroxy-10-methoxy-2,2,4-trimethyl-5H-chromeno[3,4-f]quinoline (compound 251);
(Z)-5-(2′-(3′-(4″-Oxo-piperidinecarbonyl)thienylmethylidene))1,2-dihydro-9-hydroxy-10-methoxy-2,2,4-trimethyl-5H-chromeno[3,4-f]quinoline (compound 252);
(Z)-5-(2′-(3′-(2″,2″,2″-Trifluoroethylcarbamoyl)thienylmethylidene))1,2-dihydro-9-hydroxy-10-methoxy-2,2,4-trimethyl-5H-chromeno[3,4-f]quinoline (compound 253);
(Z)-5-(2′-(3′-(4″-Hydroxypiperidinecarbonyl)thienylmethylidene))1,2-dihydro-9-hydroxy-10-methoxy-2,2,4-trimethyl-5H-chromeno[3,4-f]quinoline (compound 254);
(Z)-5-(2′-(3′-(4″-Methylpiperazinecarbonyl)thienylmethylidene))1,2-dihydro-9-hydroxy-10-methoxy-2,2,4-trimethyl-5H-chromeno[3,4-f]quinoline (compound 256);
(±)-(Z)-5-(2′-(3′-(1″-Hydroxy-4″,4″,4″-trifluorobut-2″-ynyl)thienyl-methylidene))1,2-dihydro-9-hydroxy-10-methoxy-2,2,4-trimethyl-5H-chromeno-[3,4-f]quinoline (compound 257);
(Z)-5-(2′-(3′-(3″-Hydroxy-3″-phenylpropanoyl)thienylmethylidene))1,2-dihydro-9-hydroxy-10-methoxy-2,2,4-trimethyl-5H-chromeno[3,4-f]quinoline (compound 258);
(Z)-5-(2′-(3′-(3″-Hydroxybutanoyl)thienylmethylidene))1,2-dihydro-9-hydroxy-10-methoxy-2,2,4-trimethyl-5H-chromeno[3,4-f]quinoline (compound 259); and
(Z)-5-(2′-(3′-(But-2″-enoyl)thienylmethylidene))1,2-dihydro-9-hydroxy-10-methoxy-2,2,4-trimethyl-5H-chromeno[3,4-f]quinoline (compound 260).

116. The compound of claim 1, selected from among:
(Z)-5-(2′-(3′-(dimethylaminocarbonyl)thienylmethylidene))-1,2-dihydro-9-hydroxy-10-methoxy-2,2,4-trimethyl-5H-chromeno[3,4-f]quinoline (compound 132);
(Z)-5-(2′-(3′-(pyrrolidinocarbonyl)thienylmethylidene))-1,2-dihydro-9-hydroxy-10-methoxy-2,2,4-trimethyl-5H-chromeno[3,4-f]quinoline (compound 137); and
(Z)-5-(2′-(3′-(piperidinocarbonyl)thienylmethylidene))-1,2-dihydro-9-hydroxy-10-methoxy-2,2,4-trimethyl-5H-chromeno[3,4-f]quinoline (compound 140).

117. The compound of claim 1, selected from among:
(Z)-5-(2′-(6′-methyl-pyridinylmethylidene))-1,2-dihydro-9-hydroxy-10-methoxy-2,2,4-trimethyl-5H-chromeno[3,4-f]quinoline (compound 42);
(Z)-5-(2′-(3′-(dimethylaminocarbonyl)furanylmethylidene))-1,2-dihydro-9-hydroxy-10-methoxy-2,2,4-trimethyl-5H-chromeno[3,4-f]quinoline (compound 62);
(Z)-5-(2′-(3′-(morpholinocarbonyl)thienylmethylidene)-1,2-dihydro-9-hydroxy-10-methoxy-2,2,4-trimethyl-5H-chromeno[3,4-f]quinoline (compound 135);
(Z)-5-(2′-(3′-(cyclohexylmethylaminocarbonyl)thienylmethylidene))-1,2-dihydro-9-hydroxy-10-methoxy-2,2,4-trimethyl-5H-chromeno[3,4-f]quinoline (compound 136);
(Z)-5-(2′-(3′-(di(methoxyethyl)aminocarbonyl)thienylmethylidene)-1,2-dihydro-9-hydroxy-10-methoxy-2,2,4-trimethyl-5H-chromeno[3,4-f]quinoline (compound 138);
(Z)-5-(2′-(3′-(allylmethylaminocarbonyl)thienylmethylidene))-1,2-dihydro-9-hydroxy-10-methoxy-2,2,4-trimethyl-5H-chromeno[3,4-f]quinoline (compound 139);
(Z)-5-(2′-(3′-dimethylaminocarbonyl-5′-methylfuranylmethylidene))-1,2-dihydro-9-hydroxy-10-methoxy-2,2,4-trimethyl-5H-chromeno[3,4-f]quinoline (compound 146); and
(Z)-5-(2′-(3′-cyclohexylmethylaminocarbonyl-5′-methylfuranyl-methylidene))-1,2-dihydro-9-hydroxy-10-methoxy-2,2,4-trimethyl-5H-chromeno[3,4-f]quinoline (compound 147).

118. The compound of claim 1, selected from among:
(Z)-5-(2′-(3′-Hydroxymethylthienylmethylidene))1,2-dihydro-9-hydroxy-10-methoxy-2,2,4-trimethyl-5H-chromeno[3,4-f]quinoline (compound 157);
(Z)-5-(2′-(3′-Acetylthienylmethylidene))1,2-dihydro-9-hydroxy-10-methoxy-2,2,4-trimethyl-5H-chromeno[3,4-f]quinoline (compound 168);
(±)-(Z)-5-(2′-(3′-(1″-Hydroxyethyl)thienylmethylidene))1,2-dihydro-9-hydroxy-10-methoxy-2,2,4-trimethyl-5H-chromeno[3,4-f]quinoline (compound 171);
(Z)-5-(2′-(3′-((E)-1″-Hydroxyiminoethyl)thienylmethylidene))1,2-dihydro-9-hydroxy-10-methoxy-2,2,4-trimethyl-5H-chromeno[3,4-f]quinoline (compound 174);
(Z)-5-(2′-(3′-((Z)-1″-Hydroxyiminoethyl)thienylmethylidene))1,2-dihydro-9-hydroxy-10-methoxy-2,2,4-trimethyl-5H-chromeno[3,4-f]quinoline (compound 175);
(Z)-5-(2′-(3′-((E)-1″-Methoxyiminoethyl)thienylmethylidene))1,2-dihydro-9-hydroxy-10-methoxy-2,2,4-trimethyl-5H-chromeno[3,4-f]quinoline (compound 176);
(Z)-5-(2′-(3′-((Z)-1″-Methoxyiminoethyl)thienylmethylidene))1,2-dihydro-9-hydroxy-10-methoxy-2,2,4-trimethyl-5H-chromeno[3,4-f]quinoline (compound 177);
(Z)-5-(2′-(3′-((E)-1″-Allyloxyiminoethyl)thienylmethylidene))1,2-dihydro-9-hydroxy-10-methoxy-2,2,4-trimethyl-5H-chromeno[3,4-f]quinoline (compound 178);
(Z)-5-(2′-(3′-((Z)-1″-Allyloxyiminoethyl)thienylmethylidene))1,2-dihydro-9-hydroxy-10-methoxy-2,2,4-trimethyl-5H-chromeno[3,4-f]quinoline (compound 179);
(Z)-5-(2′-(3′-((E)-1″-Phenoxyiminoethyl)thienylmethylidene))1,2-dihydro-9-hydroxy-10-methoxy-2,2,4-trimethyl-5H-chromeno[3,4-f]quinoline (compound 180);
(Z)-5-(2′-(3′-((Z)-1″-Phenoxyiminoethyl)thienylmethylidene))1,2-dihydro-9-hydroxy-10-methoxy-2,2,4-trimethyl-5H-chromeno[3,4-f]quinoline (compound 181);
(Z)-5-(2′-(3′-((E)-1″-Ethoxyiminoethyl)thienylmethylidene))1,2-dihydro-9-hydroxy-10-methoxy-2,2,4-trimethyl-5H-chromeno[3,4-f]quinoline (compound 182);
(Z)-5-(2′-(3′-((Z)-1″-Ethoxyiminoethyl)thienylmethylidene))1,2-dihydro-9-hydroxy-10-methoxy-2,2,4-trimethyl-5H-chromeno[3,4-f]quinoline (compound 183);
(Z)-5-(2′-(3′-((E)-1″-tert-Butoxyiminoethyl)thienylmethylidene))1,2-dihydro-9-hydroxy-10-methoxy-2,2,4-trimethyl-5H-chromeno[3,4-f]quinoline (compound 185);
(Z)-5-(2′-(3′-((E)-1″-Benzyloxyiminoethyl)thienylmethylidene))1,2-dihydro-9-hydroxy-10-methoxy-2,2,4-trimethyl-5H-chromeno[3,4-f]quinoline (compound 186);
(Z)-5-(2′-(3′-((Z)-1″-Benzyloxyiminoethyl)thienylmethylidene))1,2-dihydro-9-hydroxy-10-methoxy-2,2,4-trimethyl-5H-chromeno[3,4-f]quinoline (compound 187);
(Z)-5-(2′-(3′-((E)-Hydroxyiminomethyl)thienylmethylidene))1,2-dihydro-9-hydroxy-10-methoxy-2,2,4-trimethyl-5H-chromeno[3,4-f]quinoline (compound 190);
(Z)-5-(2′-(3′-Methoxymethylthienylmethylidene))1,2-dihydro-9-hydroxy-10-methoxy-2,2,4-trimethyl-5H-chromeno[3,4-f]quinoline (compound 193);
(Z)-5-(2′-(3′-(Methoxymethoxymethyl)thienylmethylidene))1,2-dihydro-9-hydroxy-10-methoxy-2,2,4-trimethyl-5H-chromeno[3,4-f]quinoline (compound 194);
(±)-(Z)-5-(2′-(3′-(1″-Hydroxy-2″,2″,2″-trifluoroethyl)thienylmethylidene))-1,2-dihydro-9-hydroxy-10-methoxy-2,2,4-trimethyl-5H-chromeno[3,4-f]quinoline (compound 202);
(+)-(Z)-5-(2′-(3′-(1″-Hydroxy-2″,2″,2″-trifluoroethyl)thienylmethylidene))-1,2-dihydro-9-hydroxy-10-methoxy-2,2,4-trimethyl-5H-chromeno[3,4-f]quinoline (compound 203);
(−)-(Z)-5-(2′-(3′-(1″-Hydroxy-2″,2″,2″-trifluoroethyl)thienylmethylidene))1,2-dihydro-9-hydroxy-10-methoxy-2,2,4-trimethyl-5H-chromeno[3,4-f]quinoline (compound 204);
(Z)-5-(2′-(3′-(Cyclohexanecarbonyl)thienylmethylidene))1,2-dihydro-9-hydroxy-10-methoxy-2,2,4-trimethyl-5H-chromeno[3,4-f]quinoline (compound 216);
(Z)-5-(2′-(3′-(Prop-2″-ynylaminomethyl)thienylmethylidene))1,2-dihydro-9-hydroxy-10-methoxy-2,2,4-trimethyl-5H-chromeno[3,4-f]quinoline (compound 220);
(Z)-5-(2′-(3′-((2″,2″,2″-Trifluoroethylamino)methyl)thienylmethylidene))1,2-dihydro-9-hydroxy-10-methoxy-2,2,4-trimethyl-5H-chromeno[3,4-f]quinoline (compound 221);
(Z)-5-(2′-(3′-(2″-Hydroxyethoxymethyl)thienylmethylidene))1,2-dihydro-9-hydroxy-10-methoxy-2,2,4-trimethyl-5H-chromeno[3,4-f]quinoline (compound 224);
(Z)-5-(2′-(3′-(2″-(E)-Cyanovinyl)thienylmethylidene))1,2-dihydro-9-hydroxy-10-methoxy-2,2,4-trimethyl-5H-chromeno[3,4-f]quinoline (compound 238);
(±)-(Z)-5-(2′-(3′-(1″-Methoxy-2″,2″,2″-trifluoroethyl)thienylmethylidene))-1,2-dihydro-9-hydroxy-10-methoxy-2,2,4-trimethyl-5H-chromeno[3,4-f]quinoline (compound 242);
(Z)-5-(2′-(3′-(4″-Oxo-piperidinecarbonyl)thienylmethylidene))1,2-dihydro-9-hydroxy-10-methoxy-2,2,4-trimethyl-5H-chromeno[3,4-f]quinoline (compound 252); and
(Z)-5-(2′-(3′-(2″,2″,2″-Trifluoroethylcarbamoyl)thienylmethylidene))1,2-dihydro-9-hydroxy-10-methoxy-2,2,4-trimethyl-5H-chromeno[3,4-f]quinoline (compound 253).

119. The compound of claim 1, selected from among:
(Z)-5-(2′-(3′-(Prop-2″-ynloxymethyl)thienylmethylidene))1,2-dihydro-9-hydroxy-10-methoxy-2,2,4-trimethyl-5H-chromeno[3,4-f]quinoline (compound 196);
(−)-(Z)-5-(2′-(3′-(1″-Hydroxybut-3″-enyl)thienylmethylidene))1,2-dihydro-9-hydroxy-10-methoxy-2,2,4-trimethyl-5H-chromeno[3,4-f]quinoline (compound 201);
(±)-(Z)-5-(2′-(3′-(Cyclohexylhydroxymethyl)thienylmethylidene))1,2-dihydro-9-hydroxy-10-methoxy-2,2,4-trimethyl-5H-chromeno[3,4-f]quinoline (compound 210);
(±)-(Z)-5-(2′-(3′-(1″-Hydroxy-2″-phenylethyl)thienylmethylidene))1,2-dihydro-9-hydroxy-10-methoxy-2,2,4-trimethyl-5H-chromeno[3,4-f]quinoline (compound 211);
(Z)-5-(2′-(3′-(Aminomethyl)thienylmethylidene))1,2-dihydro-9-hydroxy-10-methoxy-2,2,4-trimethyl-5H-chromeno[3,4-f]quinoline (compound 218);
(Z)-5-(2′-(3′-(2″,2″,2″-Trifluoro-1″-hydroxy-1″-(trifluoromethyl)ethyl)-thienylmethylidene))1,2-dihydro-9-hydroxy-10-methoxy-2,2,4-trimethyl-5H-chromeno[3,4-f]-quinoline (compound 229); and
(Z)-5-(2′-(3′-Hydroxymethylpyridinylmethylidene))1,2-dihydro-9-hydroxy-10-methoxy-2,2,4-trimethyl-5H-chromeno[3,4-f]quinoline (compound 247).

120. A pharmaceutical composition, comprising:
a compound of claim 1; and
a pharmaceutically acceptable carrier.

121. An article of manufacture, comprising:
a packaging material,
a compound of claim 1 which is effective for modulating the activity of glucocorticoid receptor, or for treatment, prevention or amelioration of one or more symptoms of glucocorticoid receptor mediated diseases or disorders, or diseases or disorders in which glucocorticoid receptor activity is implicated, within the packaging material, and
a label that indicates that the compound or composition, or pharmaceutically acceptable derivative thereof, is used for modulating the activity of glucocorticoid receptor or for treatment, prevention or amelioration of one or more symptoms of glucocorticoid receptor mediated diseases or disorders, or diseases or disorders in which glucocorticoid receptor activity is implicated.

122. A method of treating, preventing, or ameliorating the symptoms or manifestation of a disease or disorder that is modulated by or otherwise affected by glucocorticoid receptor activity or in which glucocorticoid receptor activity is implicated, comprising administering to a subject in need thereof an effective amount of a compound of claim 1, wherein the amount is effective to treat, prevent or ameliorate the symptoms or manifestation of the disease or disorder.

123. The method of claim 122 wherein the glucocorticoid receptor is in a cell.

124. The method of claim 122, wherein the disease or disorder is selected from among inflammatory disease, transplant rejection, psoriasis, dermatitis, autoimmune disorders, malignancies, acute adrenal insufficiency, congenital adrenal hyperplasia, rheumatic fever, granulomatous disease, immune proliferation/apoptosis, HPA axis suppression and regulation, hypercortisolemia, Th1/Th2 cytokine related disorders, chronic kidney disease, stroke and spinal cord injury, hypercalcemia, hyperglycemia, cerebral edema, thrombocytopenia, Little's syndrome, Addison's disease, cystic fibrosis, myasthenia gravis, autoimmune hemolytic anemia, uveitis, pemphigus vulgaris, multiple sclerosis, nasal polyps, sepsis, infectious disease, type II diabetes, obesity, metabolic syndrome, depression, schizophrenia, mood disorders, Cushing's syndrome, anxiety, sleep disorders, memory and learning enhancement, and glaucoma.

125. The method of claim 124, wherein the inflammatory disease is selected from among rheumatoid arthritis, asthma, lupus, osteoarthritis, rhinosinusitis, inflammatory bowel disease, polyarteritis nodosa, Wegener's granulomatosis, giant cell arteritis, allergic rhinitis, urticaria, hereditary angioedema, chronic obstructive pulmonary disease, tendonitis, bursitis, autoimmune chronic active hepatitis and cirrhosis.

126. The compound of claim 1, wherein the compound is a selective glucocorticoid receptor modulator.

127. The compound of claim 1 that is a selective glucocorticoid receptor agonist.

128. The compound of claim 1 that is a selective glucocorticoid receptor antagonist.

129. The compound of any claim 1 that is a selective glucocorticoid receptor partial agonist.

130. The compound of claim 1, wherein the compound is a selective glucocorticoid receptor modulator that is a tissue-specific modulator.

131. A selective glucocorticoid binding compound of claim 1.

132. A method for identifying a compound that is capable of modulating an activity of a glucocorticoid receptor, comprising:
contacting a cell expressing a glucocorticoid receptor with one or more compound(s) of claim 1; and
monitoring an effect of the compound upon the cell.

133. The method of claim 132, wherein the cell is contacted with a glucocorticoid receptor agonist prior to or simultaneously with the compound, and the compound is a glucocorticoid receptor antagonist.

134. A method of treating an individual having a disease or disorder that is modulated by glucocorticoid receptor, comprising administering to the individual a pharmaceutically effective amount of a compound of claim 1.

135. The method of claim 134, wherein the disease or disorder is selected from among inflammatory disease, transplant rejection, psoriasis, dermatitis, autoimmune disorders, malignancies, acute adrenal insufficiency, congenital adrenal hyperplasia, rheumatic fever, granulomatous disease, immune proliferation/apoptosis, HPA axis suppression and regulation, hypercortisolemia, Th1/Th2 cytokine related disorders, chronic kidney disease, stroke and spinal cord injury, hypercalcemia, hyperglycemia, cerebral edema, thrombocytopenia, Little's syndrome, Addison's disease, cystic fibrosis, myasthenia gravis, autoimmune hemolytic anemia, uveitis, pemphigus vulgaris, multiple sclerosis, nasal polyps, sepsis, infectious disease, type II diabetes, obesity, metabolic syndrome, depression, schizophrenia, mood disorders, Cushing's syndrome, anxiety, sleep disorders, memory and learning enhancement, and glaucoma.

136. The method of claim 135, wherein the inflammatory disease is selected from among rheumatoid arthritis, asthma, lupus, osteoarthritis, rhinosinusitis, inflammatory bowel disease, polyarteritis nodosa, Wegener's granulomatosis, giant cell arteritis, allergic rhinitis, urticaria, hereditary angioedema, chronic obstructive pulmonary disease, tendonitis, bursitis, autoimmune chronic active hepatitis and cirrhosis.

137. (canceled)
